SEQUENCE LISTING (4 sequences)

SEQ ID NO: 1 - protein, 1089 aa, Homo sapiens
  DOMAIN (550)..(1089): Cytoplasmic Domain
MGTSHPAFLVLGCLLTGLSLILCQLSLPSILPNENEKVVQLNSSFSLRCFGESEVSWQYPMSEEESSDVEIRNEENNSGLFVTVLEVSSASAAHTGLYTCYYNHTQTEENELEGRHIYIYVPDPDVAFVPLGMTDYLVIVEDDDSAIIPCRTTDPETPVTLHNSEGVVPASYDSRQGFNGTFTVGPYICEATVKGKKFQTIPFNVYALKATSELDLEMEALKTVYKSGETIVVTCAVFNNEVVDLQWTYPGEVKGKGITMLEEIKVPSIKLVYTLTVPEATVKDSGDYECAARQATREVKEMKKVTISVHEKGFIEIKPTFSQLEAVNLHEVKHFVVEVRAYPPPRISWLKNNLTLIENLTEITTDVEKIQEIRYRSKLKLIRAKEEDSGHYTIVAQNEDAVKSYTFELLTQVPSSILDLVDDHHGSTGGQTVRCTAEGTPLPDIEWMICKDIKKCNNETSWTILANNVSNIITEIHSRDRSTVEGRVTFAKVEETIAVRCLAKNLLGAENRELKLVAPTLRSELTVAAAVLVLLVIVIISLIVLVVIWKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIITEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 2 - protein, 774 aa, Artificial Sequence
  GST tagged, active human PDGFRa [T674I]
  MISC_FEATURE: Human PDGFRa [T674I] cytoplasmic domain expressed as N-terminal GST-fusion protein using baculovirus expression system
MAPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGAMGSKQKPRYEIRWRVIESISPDGHEYIYVDPMQLPYDSRWEFPRDGLVLGRVLGSGAFGKVVEGTAYGLSRSQPVMKVAVKMLKPTARSSEKQALMSELKIMTHLGPHLNIVNLLGACTKSGPIYIIIEYCFYGDLVNYLHKNRDSFLSHHPEKPKKELDIFGLNPADESTRSYVILSFENNGDYMDMKQADTTQYVPMLERKEVSKYSDIQRSLYDRPASYKKKSMLDSEVKNLLSDDNSEGLTLLDLLSFTYQVARGMEFLASKNCVHRDLAARNVLLAQGKIVKICDFGLARDIMHDSNYVSKGSTFLPVKWMAPESIFDNLYTTLSDVWSYGILLWEIFSLGGTPYPGMMVDSTFYNKIKSGYRMAKPDHATSEVYEIMVKCWNSEPEKRPSFYHLSEIVENLLPGQYKKSYEKIHLDFLKSDHPAVARMRVDSDNAYIGVTYKNEEDKLKDWEGGLDEQRLSADSGYIIPLPDIDPVPEEEDLGKRNRHSSQTSEESAIETGSSSSTFIKREDETIEDIDMMDDIGIDSSDLVEDSFL

SEQ ID NO: 3 - protein, 1106 aa, Homo sapiens
  DOMAIN (557)..(1106): Cytoplasmic Domain
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 4 - protein, 781 aa, Artificial Sequence
  GST tagged, active human PDGFRb
  MISC_FEATURE: Human PDGFRb cytoplasmic domain expressed as N-terminal GST-fusion protein using baculovirus expression system
MAPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLEVLFQGPLGAQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

(19) United States
(12) Patent Application Publication
(10) Pub. No.: US 2023/0102554 A1
(43) Pub. Date: Mar. 30, 2023

(54) HETEROCYCLIC KINASE INHIBITORS AND PRODUCTS AND USES THEREOF

(71) Applicant: GB002, INC., San Diego, CA (US)

(72) Inventors: Luis Lopez, San Diego, CA (US); Craig Coburn, San Diego, CA (US); Martin W. Rowbottom, San Diego, CA (US); Iriny Botrous, San Diego, CA (US); Michelle Kasem, San Diego, CA (US)

(21) Appl. No.: 17/623,562

(22) PCT Filed: Jun. 26, 2020
(86) PCT No.: PCT/US2020/039981

§ 371 Date: Dec. 28, 2021

Related U.S. Application Data
(60) Provisional application No. 62/868,735, filed on Jun. 28, 2019.

(51) Int. Cl. C07D 487/04; C07D 471/04; C07D 401/12; C07D 491/048; C07D 495/04; C07D 519/00; C07D 498/04

(52) U.S. Cl. CPC C07D 487/04; C07D 471/04; C07D 401/12; C07D 498/04; C07D 495/04; C07D 519/00; C07D 491/048

(57) ABSTRACT

Compounds are provided having the structure of Formula (I) or a pharmaceutically acceptable isomer, racemate, hydrate, solvate, tautomer, isotope, or salt thereof, wherein A, X, R³, R⁵, R⁶, R⁷, R⁸, Y², Y⁴, Y⁶, Y⁷, Y⁸, Y⁹, m, and n are as defined herein. Such compounds inhibit tyrosine kinase receptors, particularly the platelet derived growth factor receptor-alpha (PDGFR-α) and/or the platelet derived growth factor receptor-beta (PDGFR-β). Products containing such compounds, as well as methods for their use and preparation, are also provided.

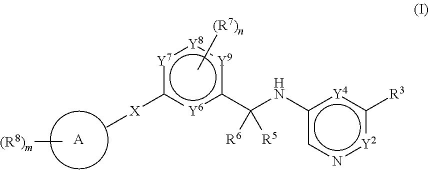

### FIELD OF THE INVENTION

The present invention relates generally to tyrosine kinase receptor modulators, and particularly to compounds that modulate the platelet derived growth factor receptor (PDGFR), as well as to products containing the same and to methods of their use and preparation.

### BACKGROUND

Receptor tyrosine kinases are transmembrane polypeptides that regulate the regeneration, remodeling, development, and differentiation of cells. Among the receptor tyrosine kinases is the platelet derived growth factor receptor (PDGFR), which is associated with pulmonary diseases, tissue fibrosis, and solid tumors.

Among the pulmonary diseases, pulmonary hypertension (PH) is a rare disorder of the pulmonary vasculature that is associated with high morbidity and mortality. The pathology of the disease includes plexiform lesions of disorganized angiogenesis and abnormal neointimal cellular proliferation, which obstruct blood flow through the pulmonary arterioles. Known kinase receptor inhibitors, and in particular known PDGFR inhibitors, are not orally available and or are associated with with off-target effects that can contribute to PH development and/or are associated with dose limiting side effects. Accordingly, there remains a need in the art for agents that can be administered orally and can inhibit PDGFRα and/or PDGFRβ with improved potency and selectivity over other kinases known to be involved with dose-limiting side effects (e.g. cKit, FLT3, and VEGFR2).

### BRIEF SUMMARY

In one embodiment, compounds are provided having the structure of Formula (I):

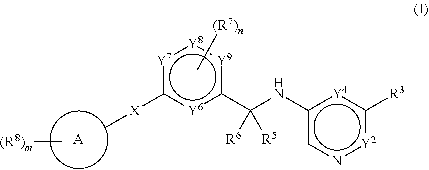

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

ring A is carbocycle or heterocycle;

Y² is a bond, —CR²═, NR², or —N═;

Y⁴ is a bond, —CR⁴═, —NR⁴—, or —N═;

R² or R⁴, together with R³ and the atoms to which they are attached, form ring B;

R² is H, halogen, cyano, alkyl, haloalkyl, alkoxy, —NH₂, —NHR⁹, or —NR⁹R⁹ when R³ and R⁴ form ring B;

R⁴ is H, halogen, cyano, alkyl, haloalkyl, alkoxy, —NH₂, —NHR⁹, or —NR⁹R⁹ when R³ and R² form ring B;

ring B is a 5- to 6-membered carbocycle or 5- to 6-membered heterocycle, wherein ring B is substituted by (R⁹)ₚ;

Y⁶, Y⁷, Y⁸, and Y⁹ are each, independently, —CH═, —CR⁷═, or N;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

R¹⁰ is H, alkyl, or haloalkyl;

R¹¹ is H, alkyl, or haloalkyl;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when ring B is a 6-membered carbocycle;

n is 0-5;

p is 0-5; and

q is 0-2.

In another embodiment, compounds are provided having the structure listed in Table 5, or a pharmaceutically acceptable isomer, racemate, hydrate, solvate, tautomer, isotope, or salt thereof.

In another embodiment, a substantially enantiomerically pure form of a compound is provided having the structure listed in Table 5 or a pharmaceutically acceptable isomer, racemate, hydrate, solvate, tautomer, isotope, or salt thereof.

In another embodiment, a composition is provided comprising a compound having the structure of any one of Formulas (I)-(XX), or a pharmaceutically acceptable isomer, racemate, hydrate, solvate, tautomer, isotope, or salt thereof, and a pharmaceutically acceptable carrier, diluent, or excipient.

In another embodiment, a method for inhibiting PDGF receptor α is provided, comprising contacting the PDGF receptor α with an effective amount of a compound having the structure of any one of Formulas (I)-(XX), or a pharmaceutically acceptable isomer, racemate, hydrate, solvate, tautomer, isotope, or salt thereof, or a composition comprising the same.

In another embodiment, a method for inhibiting PDGF receptor β is provided, comprising contacting the PDGF receptor β with an effective amount of a compound having the structure of any one of Formulas (I)-(XX), or a pharmaceutically acceptable isomer, racemate, hydrate, solvate, tautomer, isotope, or salt thereof, or a composition comprising the same.

In another embodiment, a method for treating a PDGF receptor α-dependent condition is provided, comprising administering to a subject in need thereof an effective amount of a compound having the structure of any one of Formulas (I)-(XX), or a pharmaceutically acceptable isomer, racemate, hydrate, solvate, tautomer, isotope, or salt thereof, or a composition comprising the same.

In another embodiment, a method for treating a PDGF receptor β-dependent condition is provided, comprising administering to a subject in need thereof an effective amount of a compound having the structure of any one of Formulas (I)-(XX), or a pharmaceutically acceptable isomer, racemate, hydrate, solvate, tautomer, isotope, or salt thereof, or a composition comprising the same.

In another embodiment, a method for treating a pulmonary disorder is provided, comprising administering to a subject in need thereof an effective amount of a compound having the structure of any one of Formulas (I)-(XX), or a pharmaceutically acceptable isomer, racemate, hydrate, solvate, tautomer, isotope, or salt thereof, or a composition comprising the same. In one embodiment, the pulmonary disorder is pulmonary hypertension. In a further embodiment, pulmonary hypertension is pulmonary arterial hypertension.

In another embodiment, a method for treating systemic sclerosis is provided, comprising administering to a subject in need thereof an effective amount of a compound having the structure of any one of Formulas (I)-(XX), or a pharmaceutically acceptable isomer, racemate, hydrate, solvate, tautomer, isotope, or salt thereof, or a composition comprising the same.

In another embodiment, a method for treating tissue fibrosis is provided, comprising administering to a subject in need thereof an effective amount of a compound having the structure of any one of Formulas (I)-(XX), or a pharmaceutically acceptable isomer, racemate, hydrate, solvate, tautomer, isotope, or salt thereof, or a composition comprising the same.

In another embodiment, a method for treating solid tumors is provided, comprising administering to a subject in need thereof an effective amount of a compound having the structure of any one of Formulas (I)-(XX), or a pharmaceutically acceptable isomer, racemate, hydrate, solvate, tautomer, isotope, or salt thereof, or a composition comprising the same.

### DETAILED DESCRIPTION

Unless specifically defined otherwise, the technical terms, as used herein, have their normal meaning as understood in the art. The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

As used herein, the singular terms “a,” “an,” and “the” include plural referents unless context clearly indicates otherwise. Similarly, the word “or” is intended to include “and” unless the context clearly indicates otherwise. Also, as used herein, the term “comprises” means “includes.” Thus the phrase “comprising A or B” means including A, B, or A and B.

As mentioned above, the invention relates to compounds that modulate one or both of the PDGF receptor α and the PDGF receptor β. As used herein, a “modulator” of the PDGF receptor α and the PDGF receptor β is a compound which, when administered to a subject, provides the desired modulation of the target receptor. For example, the compound may function as a full or partial antagonist or agonist of the receptor, either by interacting directly or indirectly with the target receptor.

In one embodiment, compounds are provided having the structure of Formula (I):

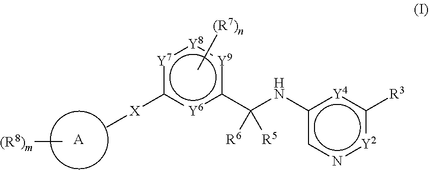

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

ring A is carbocycle or heterocycle;

Y² is a bond, —CR²═, —NR²—, or —N═;

Y⁴ is a bond, —CR⁴═, —NR⁴—, or —N═;

R² or R⁴, together with R³ and the atoms to which they are attached, form ring B;

R² is H, halogen, cyano, alkyl, haloalkyl, alkoxy, —NH₂, —NHR⁹, or —NR⁹R⁹ when R³ and R⁴ form ring B;

R⁴ is H, halogen, cyano, alkyl, haloalkyl, alkoxy, —NH₂, —NHR⁹, or —NR⁹R⁹ when R³ and R² form ring B;

ring B is a 5- to 6-membered carbocycle or 5- to 6-membered heterocycle, wherein ring B is substituted by (R⁹)ₚ;

Y⁶, Y⁷, Y⁸, and Y⁹ are each, independently, —CH═, —CR⁷═, or N;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

R¹⁰ is H, alkyl, or haloalkyl;

R¹¹ is H, alkyl, or haloalkyl;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when ring B is a 6-membered carbocycle;

n is 0-5;

p is 0-5; and

q is 0-2.

As used herein, “alkyl” means a straight chain or branched saturated hydrocarbon group. “Lower alkyl” means a straight chain or branched alkyl group having from 1 to 8 carbon atoms, in some embodiments from 1 to 6 carbon atoms, in some embodiments from 1 to 4 carbon atoms, and in some embodiments from 1 to 2 carbon atoms. Examples of straight chain lower alkyl groups include, but are not limited to, methyl, ethyl, n-propyl, n-butyl, n-pentyl, n-hexyl, n-heptyl, and n-octyl groups. Examples of branched lower alkyl groups include, but are not limited to, isopropyl, iso-butyl, sec-butyl, t-butyl, neopentyl, isopentyl, and 2,2-dimethylpropyl groups.

“Alkenyl” groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to —CH═CH₂, —CH═CH(CH₃), —CH═C(CH₃)₂, —C(CH₃)═CH₂, —C(CH₃)═CH(CH₃), —C(CH₂CH₃)═CH₂, —CH═CHCH₂CH₃, —CH═CH(CH₂)₂CH₃, —CH═CH(CH₂)₃CH₃, —CH═CH(CH₂)₄CH₃, vinyl, cyclohexenyl, cyclopentenyl, cyclohexadienyl, butadienyl, pentadienyl, and hexadienyl among others.

“Alkynyl” groups include straight and branched chain alkyl groups, except that at least one triple bond exists between two carbon atoms. Thus, alkynyl groups have from 2 to about 20 carbon atoms, and typically from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃), among others.

As used herein, “alkylene” means a divalent alkyl group. Examples of straight chain lower alkylene groups include, but are not limited to, methylene (i.e., —CH₂—), ethylene (i.e., —CH₂CH₂—), propylene (i.e., —CH₂CH₂CH₂—), and butylene (i.e., —CH₂CH₂CH₂CH₂—). As used herein, “heteroalkylene” is an alkylene group of which one or more carbon atoms is replaced with a heteroatom such as, but not limited to, N, O, S, or P.

“Alkoxy” refers to an alkyl as defined above joined by way of an oxygen atom (i.e., —O-alkyl). Examples of lower alkoxy groups include, but are not limited to, methoxy, ethoxy, n-propoxy, n-butoxy, isopropoxy, sec-butoxy, tert-butoxy, and the like.

The terms “carbocyclic” and “carbocycle” denote a ring structure wherein the atoms of the ring are carbon. Carbocycles may be monocyclic or polycyclic. Carbocycle encompasses both saturated and unsaturated rings. Carbocycle encompasses both fused and spirocyclic rings. Carbocycle encompasses both cycloalkyl and aryl groups. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N substituents wherein N is the size of the carbocyclic ring with for example, alkyl, amino, hydroxy, cyano, carboxy, nitro, thio, alkoxy, and halogen groups.

“Cycloalkyl” groups are alkyl groups forming a ring structure, which can be substituted or unsubstituted. Examples of cycloalkyl include, but are not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups.

“Aryl” groups are cyclic aromatic hydrocarbons that do not contain heteroatoms. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain 6-14 carbons in the ring portions of the groups. The terms “aryl” and “aryl groups” include include fused rings wherein at least one ring, but not necessarily all rings, are aromatic, such as fused aromatic-aliphatic ring systems (e.g., indanyl, tetrahydronaphthyl, and the like).

“Carbocyclealkyl” refers to an alkyl as defined above with one or more hydrogen atoms replaced with carbocycle. Examples of carbocyclealkyl groups include, but are not limited to benzyl and the like.

As used herein, “heterocycle” or “heterocyclyl” groups include aromatic and non-aromatic ring compounds (heterocyclic rings) containing 3 or more ring members, of which one or more is a heteroatom such as, but not limited to, N, O, S, or P. A heterocycle group as defined herein can be a heteroaryl group or a partially or completely saturated cyclic group including at least one ring heteroatom. In some embodiments, heterocycle groups include 3 to 20 ring members, whereas other such groups have 3 to 15 ring members. At least one ring contains a heteroatom, but every ring in a polycyclic system need not contain a heteroatom. Heterocycle encompasses both fused and spirocyclic rings. For example, a dioxolanyl ring and a benzdioxolanyl ring system (methylenedioxyphenyl ring system) are both heterocycle groups within the meaning herein. A heterocycle group designated as a C₂-heterocycle can be a 5-membered ring with two carbon atoms and three heteroatoms, a 6-membered ring with two carbon atoms and four heteroatoms and so forth. Likewise a C₄-heterocycle can be a 5-membered ring with one heteroatom, a 6-membered ring with two heteroatoms, and so forth. The number of carbon atoms plus the number of heteroatoms sums up to equal the total number of ring atoms. A saturated heterocyclic ring refers to a heterocyclic ring containing no unsaturated carbon atoms.

“Heteroaryl” groups are aromatic ring compounds containing 5 or more ring members, of which, one or more is a heteroatom such as, but not limited to, N, O, and S. A heteroaryl group designated as a C₂-heteroaryl can be a 5-membered ring with two carbon atoms and three heteroatoms, a 6-membered ring with two carbon atoms and four heteroatoms and so forth. Likewise a C₄-heteroaryl can be a 5-membered ring with one heteroatom, a 6-membered ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, tetrahydroisoquinolinyl, quinoxalinyl, and quinazolinyl groups. The terms “heteroaryl” and “heteroaryl groups” include fused ring compounds such as wherein at least one ring, but not necessarily all rings, are aromatic, including tetrahydroquinolinyl, tetrahydroisoquinolinyl, indolyl and 2,3-dihydro indolyl.

“Heterocyclealkyl” refers to an alkyl as defined above with one or more hydrogen atoms replaced with heterocycle. Examples of heterocyclealkyl groups include, but are not limited to morpholinoethyl and the like.

“Halo” or “halogen” refers to fluorine, chlorine, bromine and iodine.

“Haloalkyl” refers to an alkyl as defined above with one or more hydrogen atoms replaced with halogen. Examples of lower haloalkyl groups include, but are not limited to, —CF₃, —CH₂CF₃, and the like.

“Haloalkoxy” refers to an alkoxy as defined above with one or more hydrogen atoms replaced with halogen. Examples of lower haloalkoxy groups include, but are not limited to —OCF₃, —OCH₂CF₃, and the like.

“Hydroxyalkyl” refers to an alkyl as defined above with one or more hydrogen atoms replaced with —OH. Examples of lower hydroxyalkyl groups include, but are not limited to —CH₂OH, —CH₂CH₂OH, and the like.

As used herein, the term “optionally substituted” refers to a group (e.g., an alkyl, carbocycle, or heterocycle) having 0, 1, or more substituents, such as 0-25, 0-20, 0-10 or 0-5 substituents. Substituents include, but are not limited to —OR^{a}, —NR^{a}R^{b}, —S(O)₂R^{a} or —S(O)₂OR^{a}, halogen, cyano, alkyl, haloalkyl, alkoxy, carbocycle, heterocycle, carbocyclalkyl, or heterocyclealkyl, wherein each R^{a} and R^{b} is, independently, H, alkyl, haloalkyl, carbocycle, or heterocycle, or R^{a} and R^{b}, together with the atom to which they are attached, form a 3-8 membered carbocycle or heterocycle.

In one embodiment, compounds are provided having the structure of Formula (I):

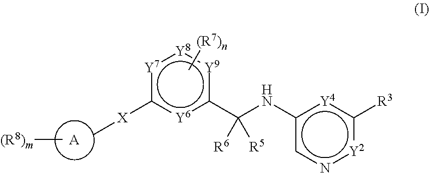

or a pharmaceutically acceptable isomer, racemate, hydrate, solvate, tautomer, isotope, or salt thereof, wherein A, X, R³, R⁵, R⁶, R⁷, R⁸, R⁹, Y², Y⁴, Y⁶, Y⁷, Y⁸, Y⁹, m, and n are as defined above. In one embodiment, ring A is a monocyclic carbocycle. In another embodiment, ring A is a polycyclic carbocycle. In one embodiment, ring A is a monocyclic heterocycle. In another embodiment, ring A is a polycyclic heterocycle.

In one embodiment, compounds are provided having the structure of Formula (I), or a pharmaceutically acceptable isomer, racemate, hydrate, solvate, tautomer, isotope, or salt thereof, wherein ring A is a monocyclic heterocycle. In one embodiment, ring A is pyrrolidinyl, pyrrolyl, pyrazolyl, imidazolyl, triazolyl, tetrazolyl, furanyl, oxazolyl, isoxazolyl, oxadiazolyl, oxatriazolyl, thiophenyl, thiazolyl, isothiazolyl, thiadiazolyl, thiatriazolyl, piperidinyl, piperazinyl, pyridinyl, pyrazinyl, pyrimidinyl, pyridazinyl, triazinyl, 2H-pyranyl, tetrahydro-2H-pyranyl, or morpholinyl.

In one embodiment, compounds are provided having the structure of Formula (I), or a pharmaceutically acceptable isomer, racemate, hydrate, solvate, tautomer, isotope, or salt thereof, wherein ring A is a polycyclic heterocycle. In one embodiment, ring A is indolyl, benzimidazolyl, indazolyl, benzotriazolyl, 1H-pyrrolo [3,2-b]pyridinyl, 1H-pyrrolo[3,2-c]pyridinyl, pyrrolo[2,3-b]pyridinyl, pyrrolo[2,3-c]pyridinyl, [1,2,3]triazolo[4,5-b]pyridinyl, 7H-pyrrolo[2,3-d]pyrimidinyl, 5H-pyrrolo[3,2-d]pyrimidinyl, 7H-purinyl, indolizinyl, pyrrolo[1,2-a]pyrimidinyl, pyrrolo[1,2-a]pyrazinyl, pyrrolo[1,2-c]pyriminyl, pyrrolo[1,2-b]pyridazinyl, imidazo[4,5-b]pyridinyl, pyrazolo[1,5-a]pyridinyl, imidazo[1,5-a]pyridinyl, imidazo[1,5-b]pyridazinyl, imidazo[1,2-a]pyridinyl, imidazo[1,2-b]pyridazinyl, imidazo[1,2-c]pyrimidinyl, imidazo[1,2-a]pyrimidinyl, [1,2,4]triazolo[4,3-a]pyridinyl, [1,2,3-triazolo[1,5-a]pyridinyl, quinolinyl, isoquinolinyl, cinnolinyl, quinazolinyl, quinoxalinyl, phthalazinyl, pyrido[2,3-b]pyrazinyl, pteridinyl, pyrido[3,4-d]pyridazinyl, 1,6-naphthyridinyl, 1,8-naphthyridinyl, 9H-carbazolyl, benzoxazolyl, dibenzofuranyl, benzothiphenyl, or dibenzothiophenyl.

In one embodiment, compounds are provided having the structure of Formula (I), wherein Y² is C, or a pharmaceutically acceptable isomer, racemate, hydrate, solvate, tautomer, isotope, or salt thereof. In another embodiment, Y² is N. In another embodiment, Y² is a bond.

In one embodiment, compounds are provided having the structure of Formula (I), wherein Y⁴ is C, or a pharmaceutically acceptable isomer, racemate, hydrate, solvate, tautomer, isotope, or salt thereof. In another embodiment, Y⁴ is N. In another embodiment, Y⁴ is a bond.

In one embodiment, compounds are provided wherein R² and R³, together with the atoms to which they are attached, form ring B and having the structure of Formula (II):

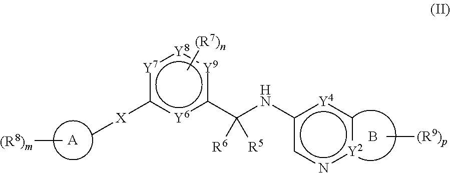

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

ring A is carbocycle or heterocycle;

Y² is C or N;

Y⁴ is a bond, —CR⁴═, or —N═;

R⁴ is H, halogen, cyano, alkyl, haloalkyl, alkoxy, —NH₂, —NHR⁹, or —NR⁹R⁹;

ring B is a 5- to 6-membered carbocycle or 5- to 6-membered heterocycle;

Y⁶, Y⁷, Y⁸, and Y⁹ are each, independently, —CH═, —CR⁷═, or N;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

R¹⁰ is H, alkyl, or haloalkyl;

R¹¹ is H, alkyl, or haloalkyl;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when ring B is a 6-membered carbocycle;

n is 0-5;

p is 0-5; and

q is 0-2.

In one embodiment, compounds are provided having the structure of Formula (III):

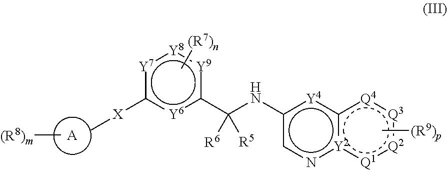

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

ring A is carbocycle or heterocycle;

Y² is C or N;

Y⁴ is a bond, —CR⁴═, or —N═;

R⁴ is H, halogen, cyano, alkyl, haloalkyl, alkoxy, —NH₂, —NHR⁹, or —NR⁹R⁹;

Q¹ and Q² are each, independently, C or N;

Q³ and Q⁴ are each, independently, a bond, C, N, S, or O;

Y⁶, Y⁷, Y⁸, and Y⁹ are each, independently, —CH═, —CR⁷═, or N;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

R¹⁰ is H, alkyl, or haloalkyl;

R¹¹ is H, alkyl, or haloalkyl;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when Q¹, Q², Q³, and Q⁴ are each C;

n is 0-5;

p is 0-5; and

q is 0-2.

In one embodiment, compounds are provided having the structure of Formula (IV):

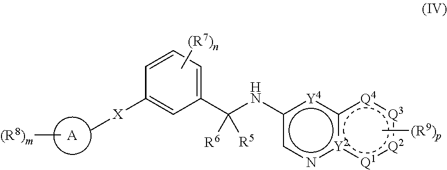

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

ring A is carbocycle or heterocycle;

Y² is C or N;

Y⁴ is a bond, —CR⁴═, or —N═;

R⁴ is H, halogen, cyano, alkyl, haloalkyl, alkoxy, —NH₂, —NHR⁹, or —NR⁹R⁹;

Q¹ and Q² are each, independently, C or N;

Q³ and Q⁴ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

R¹⁰ is H, alkyl, or haloalkyl;

R¹¹ is H, alkyl, or haloalkyl;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when Q¹, Q², Q³, and Q⁴ are each C;

n is 0-5;

p is 0-5; and

q is 0-2.

In one embodiment, compounds are provided having the structure of Formula (V):

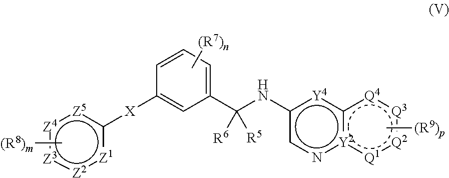

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y² is C or N;

Y⁴ is a bond, —CR⁴═, or —N═;

R⁴ is H, halogen, cyano, alkyl, haloalkyl, alkoxy, —NH₂, —NHR⁹, or —NR⁹R⁹;

Q¹ and Q² are each, independently, C or N;

Q³ and Q⁴ are each, independently, C, N, S, or O; Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

R¹⁰ is H, alkyl, or haloalkyl;

R¹¹ is H, alkyl, or haloalkyl;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, for, C₄₋₈ cycloalkyl, or 4- to 8-membered saturated heterocycle;

m is 0-5, wherein m is 1-5 when Q¹, Q², Q³, and Q⁴ are each C;

n is 0-5;

p is 0-5; and

q is 0-2.

In one embodiment, compounds are provided having the structure of Formula (VI):

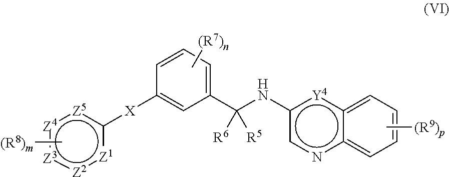

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y⁴ is a bond, —CR⁴═, or —N═;

R⁴ is H, halogen, cyano, alkyl, haloalkyl, alkoxy, —NH₂, —NHR⁹, or —NR⁹R⁹;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

R¹⁰ is H, alkyl, or haloalkyl;

R¹¹ is H, alkyl, or haloalkyl;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when Z³ is N;

n is 0-5;

p is 0-5; and

q is 0-2.

In one embodiment, compounds are provided wherein Y² is C and Q⁴ is a bond and having the structure of Formula (VII):

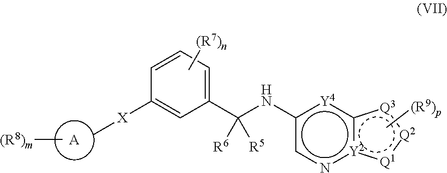

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

ring A is carbocycle or heterocycle;

Y² is C or N;

Y⁴ is a bond, —CR⁴═, or —N═;

R⁴ is H, halogen, cyano, alkyl, haloalkyl, alkoxy, —NH₂, —NHR⁹, or —NR⁹R⁹;

Q¹ and Q² are each, independently, C or N; Q³ is C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

R¹⁰ is H, alkyl, or haloalkyl;

R¹¹ is H, alkyl, or haloalkyl;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5;

n is 0-5;

p is 0-5; and

q is 0-2.

In one embodiment, compounds are provided having the structure of Formula (VIII):

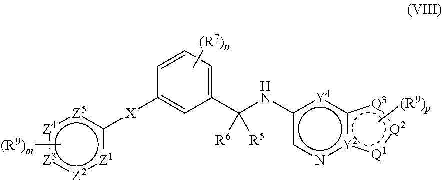

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y⁴ is a bond, —CR⁴═, or —N═;

R⁴ is H, halogen, cyano, alkyl, haloalkyl, alkoxy, —NH₂, —NHR⁹, or —NR⁹R⁹;

Q¹ and Q² are each, independently, C or N;

Q³ is C, N, S, or O;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

R¹⁰ is H, alkyl, or haloalkyl;

R¹¹ is H, alkyl, or haloalkyl;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5;

n is 0-5;

p is 0-5; and

q is 0-2.

In one embodiment, compounds are provided having the structure of Formula (VIII-A):

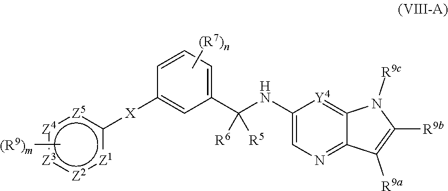

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y⁴ is —CR⁴═, or —N═;

R⁴ is H, halogen, cyano, alkyl, haloalkyl, alkoxy, —NH₂, —NHR⁹, or —NR⁹R⁹;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R^{9a} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;

R^{9b} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;

R^{9c} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;

R¹⁰ is H, alkyl, or haloalkyl;

R¹¹ is H, alkyl, or haloalkyl;

wherein R⁷, R⁸, R^{9a}, R^{9b}, and R^{9c} are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5;

n is 0-5; and

q is 0-2.

In one embodiment, compounds are provided having the structure of Formula (VIII-B):

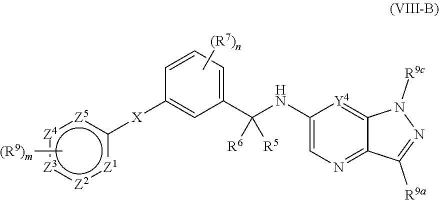

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y⁴ is —CR⁴═, or —N═;

R⁴ is H, halogen, cyano, alkyl, haloalkyl, alkoxy, —NH₂, —NHR⁹, or —NR⁹R⁹;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R^{9a} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;

R^{9c} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;

R¹⁰ is H, alkyl, or haloalkyl;

R¹¹ is H, alkyl, or haloalkyl;

wherein R⁷, R⁸, R^{9a}, and R^{9c} are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5;

n is 0-5; and

q is 0-2.

In one embodiment, compounds are provided having the structure of Formula (VIII-C):

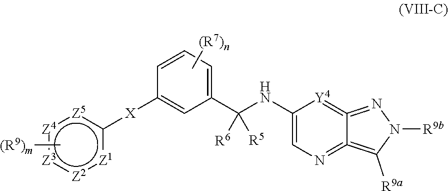

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y⁴ is —CR⁴═, or —N═;

R⁴ is H, halogen, cyano, alkyl, haloalkyl, alkoxy, —NH₂, —NHR⁹, or —NR⁹R⁹;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R^{9a} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;

R^{9b} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;

R¹⁰ is H, alkyl, or haloalkyl;

R¹¹ is H, alkyl, or haloalkyl;

wherein R⁷, R⁸, R^{9a}, and R^{9b} are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5;

n is 0-5; and

q is 0-2.

In one embodiment, compounds are provided having the structure of Formula (VIII-D):

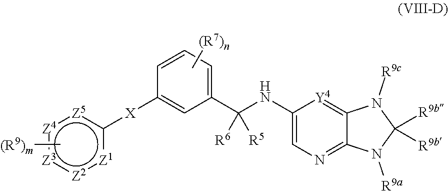

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y⁴ is —CR⁴═, or —N═;

R⁴ is H, halogen, cyano, alkyl, haloalkyl, alkoxy, —NH₂, —NHR⁹, or —NR⁹R⁹;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R^{9a} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;

R^{9b′}, and R^{9b″} are each, independently, halogen, cyano, alkyl, alkenyl, alkynyl, carbocycle, heterocycle, carbocyclealkyl, heterocyclealkyl, or R^{9b′} and R^{9b″} together form ═O, or R^{9b′} and R^{9b″} together with the carbon to which they are attached form a 3-7 membered carbocycle or heterocycle;

R^{9c} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;

R¹⁰ is H, alkyl, or haloalkyl;

R¹¹ is H, alkyl, or haloalkyl;

wherein R⁷, R⁸, R^{9a}, R^{9b′}, R^{9b″}, and R^{9c} are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5;

n is 0-5; and

q is 0-2.

In one embodiment, compounds are provided having the structure of Formula (VIII-E):

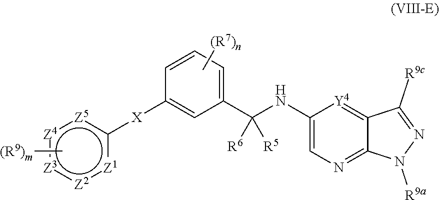

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y⁴ is —CR⁴═, or —N═;

R⁴ is H, halogen, cyano, alkyl, haloalkyl, alkoxy, —NH₂, —NHR⁹, or —NR⁹R⁹;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R^{9a} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;

R^{9c} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;

R¹⁰ is H, alkyl, or haloalkyl;

R¹¹ is H, alkyl, or haloalkyl;

wherein R⁷, R⁸, R^{9a}, and R^{9c} are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5;

n is 0-5; and

q is 0-2.

In one embodiment, compounds are provided having the structure of Formula (VIII-F):

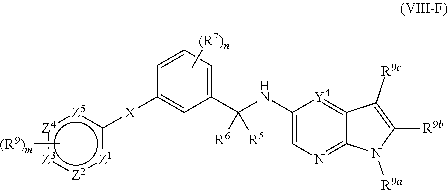

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y⁴ is —CR⁴═, or —N═;

R⁴ is H, halogen, cyano, alkyl, haloalkyl, alkoxy, —NH₂, —NHR⁹, or —NR⁹R⁹;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R^{9a} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;

R^{9b} and R^{9c} are each, independently, H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;

R¹⁰ is H, alkyl, or haloalkyl;

R¹¹ is H, alkyl, or haloalkyl;

wherein R⁷, R⁸, R^{9a}, R^{9b}, and R^{9c} are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5;

n is 0-5; and

q is 0-2.

In one embodiment, compounds are provided having the structure of Formula (VIII-G):

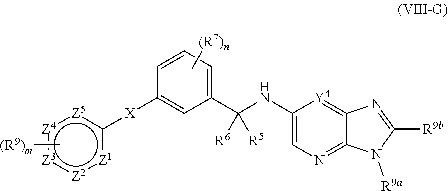

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y⁴ is —CR⁴═, or —N═;

R⁴ is H, halogen, cyano, alkyl, haloalkyl, alkoxy, —NH₂, —NHR⁹, or —NR⁹R⁹;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R^{9a} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;

R^{9b} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;

R¹⁰ is H, alkyl, or haloalkyl;

R¹¹ is H, alkyl, or haloalkyl;

wherein R⁷, R⁸, R^{9a}, and R^{9b} are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5;

n is 0-5; and

q is 0-2.

In one embodiment, compounds are provided having the structure of Formula (VIII-H):

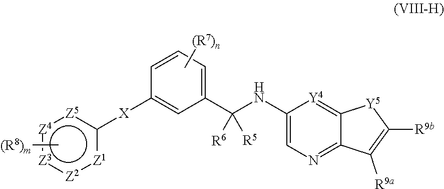

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y⁴ is —CR⁴═, or —N═;

Y⁵ is O, or S;

R⁴ is H, halogen, cyano, alkyl, haloalkyl, alkoxy, —NH₂, —NHR⁹, or —NR⁹R⁹;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R^{9a} and R^{9b} are each, independently, H, halogen, cyano, —OR^{a}, —S(O)_{q}R^{a}, —S(O)_{q}NR^{a}R^{b}, —NR^{a}S(O)_{c}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;

R¹⁰ is H, alkyl, or haloalkyl;

R¹¹ is H, alkyl, or haloalkyl;

wherein R⁷, R⁸, R^{9a}, and R^{9b} are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5;

n is 0-5; and

q is 0-2.

In one embodiment, compounds are provided wherein Y⁴ is a bond and having the structure of Formula (IX):

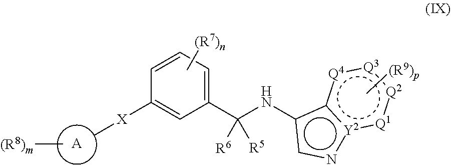

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

ring A is carbocycle or heterocycle;

Y² is C or N;

Q¹ and Q² are each, independently, C or N;

Q³ and Q⁴ are each, independently, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when Q¹, Q², Q³, and Q⁴ are each C;

n is 0-5;

p is 0-5; and

q is 0-2.

In one embodiment, compounds are provided having the structure of Formula (X):

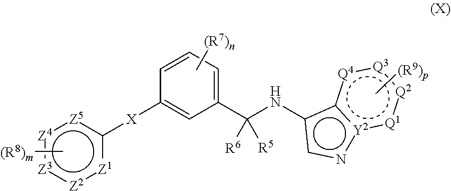

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y² is C or N;

Q¹ and Q² are each, independently, C or N;

Q³ and Q⁴ are each, independently, C, N, S, or O;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when Q¹, Q², Q³, and Q⁴ are each C;

n is 0-5;

p is 0-5; and

q is 0-2.

In one embodiment, compounds are provided having the structure of Formula (XI):

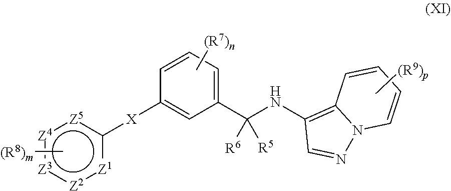

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 1-5;

n is 0-5;

p is 0-5; and

q is 0-2.

In one embodiment, compounds are provided having the structure of Formula (XII):

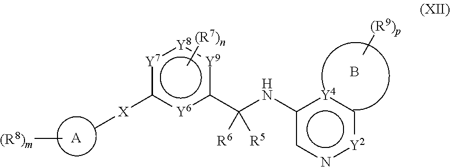

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

ring A is carbocycle or heterocycle;

Y² is a bond, —CR²═, or —N═;

Y⁴ is C or N;

R² is H, halogen, cyano, alkyl, haloalkyl, alkoxy, —NH₂, —NHR⁹, or —NR⁹R⁹;

ring B is a 5- to 6-membered carbocycle or 5- to 6-membered heterocycle, wherein ring B is substituted by (R⁹)ₚ;

Y⁶, Y⁷, Y⁸, and Y⁹ are each, independently, —CH═, —CR⁷═, or N;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when ring B is a 6-membered carbocycle;

n is 0-5;

p is 0-5; and

q is 0-2.

In one embodiment, compounds are provided having the structure of Formula (XIII):

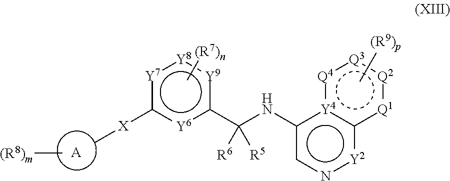

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

ring A is carbocycle or heterocycle;

Y² is a bond, —CR²═, or —N═;

Y⁴ is C or N;

R² is H, halogen, cyano, alkyl, haloalkyl, alkoxy, —NH₂, —NHR⁹, or —NR⁹R⁹;

Q¹ and Q² are each, independently, C or N;

Q³ and Q⁴ are each, independently, a bond, C, N, S, or O;

Y⁶, Y⁷, Y⁸, and Y⁹ are each, independently, —CH═, —CR⁷═, or N;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when Q¹, Q², Q³, and Q⁴ are each C;

n is 0-5;

p is 0-5; and

q is 0-2.

In one embodiment, compounds are provided having the structure of Formula (XIV):

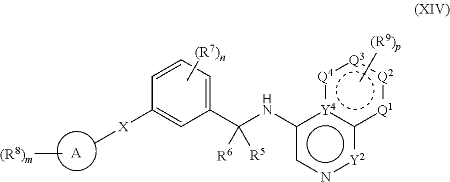

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

ring A is carbocycle or heterocycle;

Y² is a bond, —CR²═, or —N═;

Y⁴ is C or N;

R² is H, halogen, cyano, alkyl, haloalkyl, alkoxy, —NH₂, —NHR⁹, or —NR⁹R⁹;

Q¹ and Q² are each, independently, C or N;

Q³ and Q⁴ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when Q¹, Q², Q³, and Q⁴ are each C;

n is 0-5;

p is 0-5; and

q is 0-2.

In one embodiment, compounds are provided having the structure of Formula (XV):

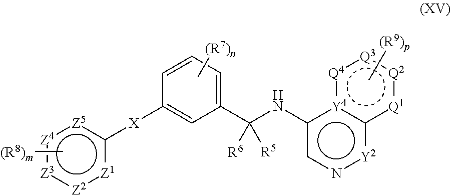

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y² is a bond, —CR²═, or —N═;

Y⁴ is C or N;

R² is H, halogen, cyano, alkyl, haloalkyl, alkoxy, —NH₂, —NHR⁹, or —NR⁹R⁹;

Q¹ and Q² are each, independently, C or N;

Q³ and Q⁴ are each, independently, C, N, S, or O;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when Q¹, Q², Q³, and Q⁴ are each C;

n is 0-5;

p is 0-5; and

q is 0-2.

In one embodiment, compounds are provided having the structure of Formula (XVI):

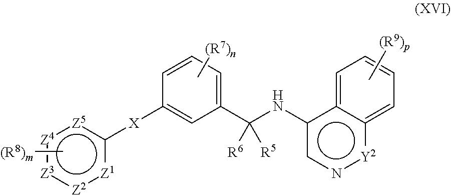

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y² is a bond, —CR²═, or —N═;

R² is H, halogen, cyano, alkyl, haloalkyl, alkoxy, —NH₂, —NHR⁹, or —NR⁹R⁹;

Q¹ and Q² are each, independently, C or N;

Q³ and Q⁴ are each, independently, C, N, S, or O;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when Q¹, Q², Q³, and Q⁴ are each C;

n is 0-5;

p is 0-5; and

q is 0-2.

In one embodiment, compounds are provided having the structure of Formula (XVII):

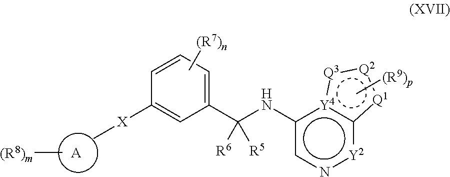

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

ring A is carbocycle or heterocycle;

Y² is a bond, —CR²═, or —N═;

Y⁴ is C or N;

R² is H, halogen, cyano, alkyl, haloalkyl, alkoxy, —NH₂, —NHR⁹, or —NR⁹R⁹;

Q¹ and Q² are each, independently, C or N;

Q³ is C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5;

n is 0-5;

p is 0-5; and

q is 0-2.

In one embodiment, compounds are provided having the structure of Formula (XVIII):

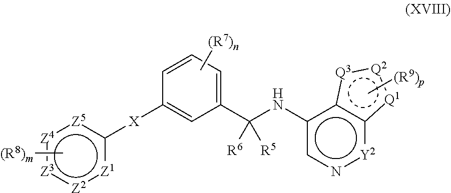

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y² is a bond, —CR²═, or —N═;

R² is H, halogen, cyano, alkyl, haloalkyl, alkoxy, —NH₂, —NHR⁹, or —NR⁹R⁹;

Q¹ and Q² are each, independently, C or N;

Q³ is C, N, S, or O;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5;

n is 0-5;

p is 0-5; and

q is 0-2.

In one embodiment, compounds are provided having the structure of Formula (XVIII-A):

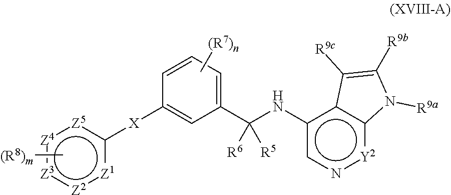

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y² is a bond, —CR²═, or —N═;

R² is H, halogen, cyano, alkyl, haloalkyl, alkoxy, —NH₂, —NHR⁹, or —NR⁹R⁹;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R^{9a} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;

R^{9b} and R^{9c} are each, independently, H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;

wherein R⁷, R⁸, R^{9a}, R^{9b}, and R^{9c} are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5;

n is 0-5; and

q is 0-2.

In one embodiment, compounds are provided having the structure of Formula (XVIII-B):

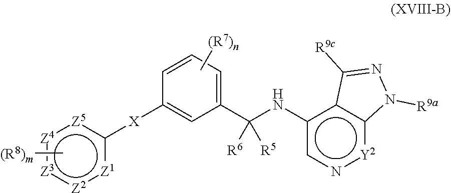

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y² is a bond, —CR²═, or —N═;

R² is H, halogen, cyano, alkyl, haloalkyl, alkoxy, —NH₂, —NHR⁹, or —NR⁹R⁹;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl;

R^{9a} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;

R^{9c} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;

wherein R⁷, R⁸, and R⁹, R^{9a}, and R^{9c} are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5;

n is 0-5; and

q is 0-2.

In one embodiment, compounds are provided having the structure of Formula (XIX):

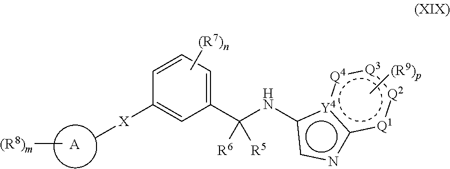

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

ring A is carbocycle or heterocycle;

Y⁴ is C or N;

Q¹ and Q² are each, independently, C or N;

Q³ and Q⁴ are each, independently, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

wherein R⁷, R⁸, and R⁹ are each, independently, optionally substituted with one or more R;

R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when Q¹, Q², Q³, and Q⁴ are each C;

n is 0-5;

p is 0-5; and

q is 0-2.

In one embodiment, compounds are provided having the structure of Formula (XX):

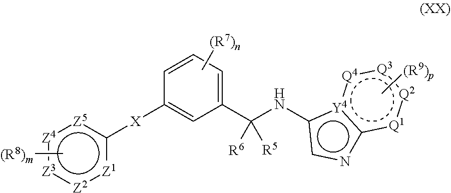

or a pharmaceutically acceptable isomer, racemate, hydrate, solvate, tautomer, isotope, or salt thereof, wherein:

X is —C(O)NH—, —C(R¹⁰R¹¹)C(O)NH—, —NHC(O)NH—, or —NHC(O)—;

Y⁴ is C or N;

Q¹ and Q² are each, independently, C or N;

Q³ and Q⁴ are each, independently, C, N, S, or O;

Z¹, Z², Z³, Z⁴, and Z⁵ are each, independently, a bond, C, N, S, or O;

R⁵ is H, alkyl, haloalkyl, or hydroxyalkyl;

R⁶ is H, alkyl, haloalkyl, or hydroxyalkyl;

or R⁵ and R⁶, together with the carbon atom they are attached to, form a 3- to 5-membered carbocycle or heterocycle;

R⁷ is, at each occurrence, independently halogen, cyano, alkyl, haloalkyl, alkoxy, or haloalkoxy;

or R⁶ and one R⁷, together with the atoms to which they are attached, form a 5- to 6-membered carbocycle;

R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;

R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;

wherein R⁷, R⁸, and R⁹ are each, independently, optionally substituted with one or more R;

R is —OR a, —C(O)R^{a}, —NR^{a}R^{b}, halogen, cyano, alkyl, haloalkyl, alkoxy, carbocycle, heterocycle, carbocyclalkyl, or heterocyclealkyl;

R^{a} is H, alkyl, haloalkyl, carbocycle, heterocycle, carbocyclealkyl, or heterocyclealkyl;

R^{b} is H, alkyl, haloalkyl, carbocycle, heterocycle, carbocyclealkyl, or heterocyclealkyl;

or R^{a} and R^{b}, together with the nitrogen atom to which they are attached, form C₄₋₈ cycloalkyl, or 4- to 8-membered saturated heterocycle;

m is 0-5, wherein m is 1-5 when Q¹, Q², Q³, and Q⁴ are each C;

n is 0-5;

p is 0-5; and

q is 0-2.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein X is —C(O)NH—. In specific embodiments, compounds of Formulas (VIII-A), (VIII-B), (VIII-C), (VIII-D), (VIII-E), (VIII-F), (VIII-G), (VIII-H), (XI), (XVI), (XVIII-A), (XVIII-B), and (XX) are provided having the structure of any one of Formulas (VIII-A-1), (VIII-B-1), (VIII-C-1), (VIII-D-1), (VIII-E-1), (VIII-F-1), (VIII-G-1), (VIII-H-1), (XI-1), (XVI-1), (XVIII-A-1), (XVIII-B-1), or (XX-1) as shown in Table 1, below:

TABLE 1
EMBODIMENTS WHERE X IS —C(O)NH—

| Formula | Structure |
|---|---|
| (VIII-A-1) | 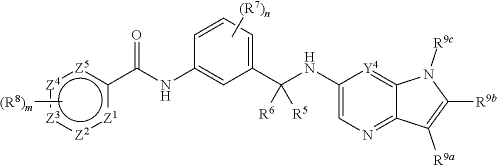 |
| (VIII-B-1) | 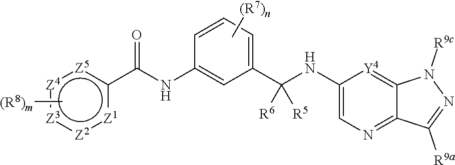 |
| (VIII-C-1) | 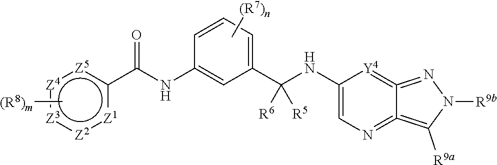 |
| (VIII-D-1) | 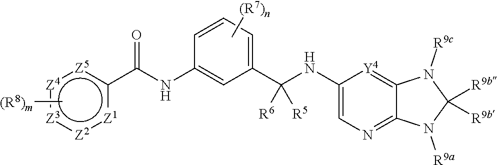 |
| (VIII-E-1) | 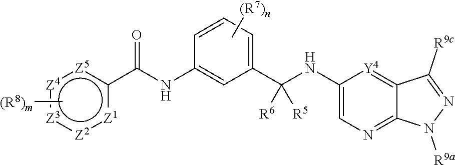 |
| (VIII-F-1) | 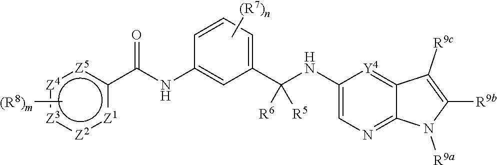 |
| (VIII-G-1) | 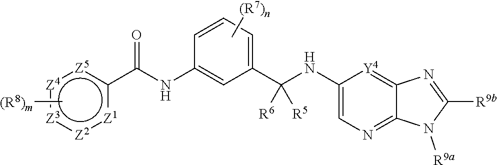 |
| (VIII-H-1) | 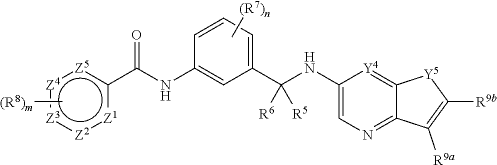 |
| (XI-1) | 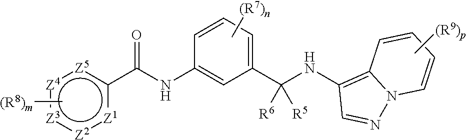 |
| (XVI-1) | 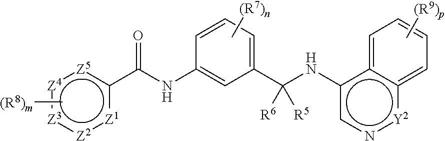 |
| (XVIII-A-1) | 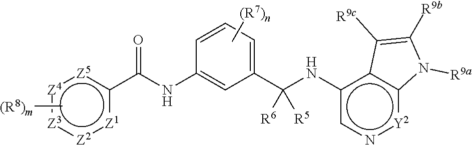 |
| (XVIII-B-1) | 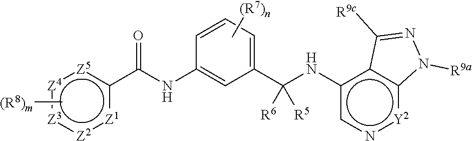 |
| (XX-1) | 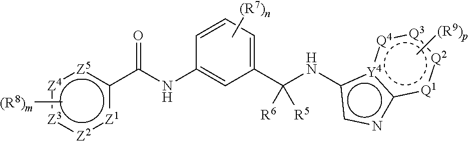 |

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein X is —C(R¹⁰R¹¹)C(O)NH—. In specific embodiments, compounds of Formulas (VIII-A), (VIII-B), (VIII-C), (VIII-D), (VIII-E), (VIII-F), (VIII-G), (VIII-H), (XI), (XVI), (XVIII-A), (XVIII-B), and (XX) are provided having the structure of any one of Formulas (VIII-A-2), (VIII-B-2), (VIII-C-2), (VIII-D-2), (VIII-E-2), (VIII-F-2), (VIII-G-2), (VIII-H-2), (XI-2), (XVI-2), (XVIII-A-2), (XVIII-B-2), or (XX-2) as shown in Table 2, below:

TABLE 2
EMBODIMENTS WHERE X IS —C(R¹⁰R¹¹)C(O)NH—

| Formula | Structure |
|---|---|
| (VIII-A-2) | 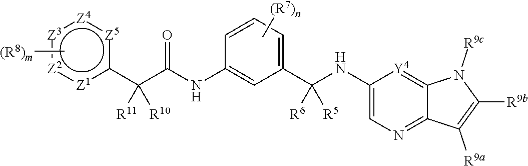 |
| (VIII-B-2) | 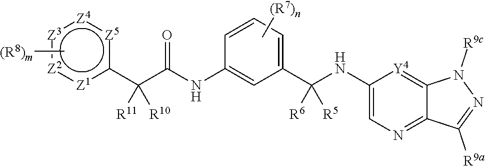 |
| (VIII-C-2) | 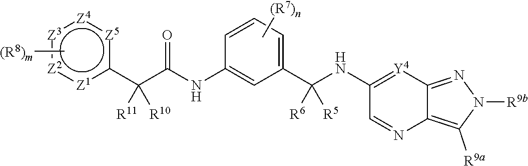 |
| (VIII-D-2) | 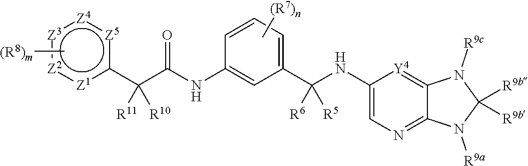 |
| (VIII-E-2) | 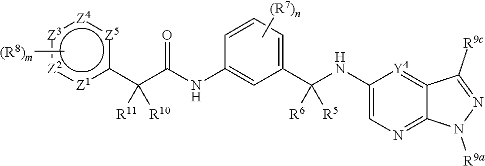 |
| (VIII-F-2) | 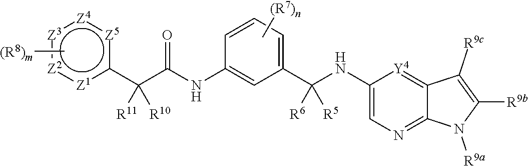 |
| (VIII-G-2) | 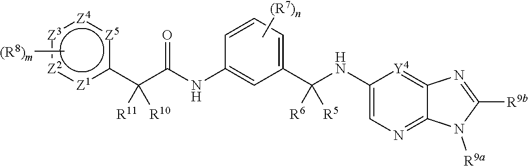 |
| (VIII-H-2) | 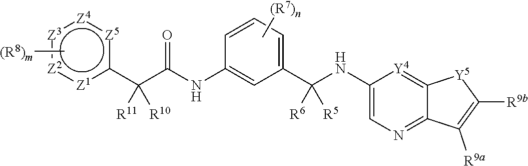 |
| (XI-2) | 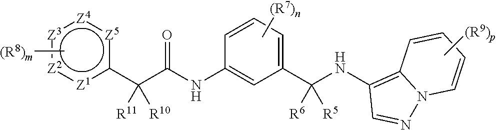 |
| (XVI-2) | 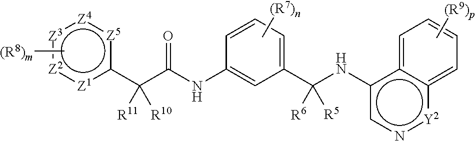 |
| (XVIII-A-2) | 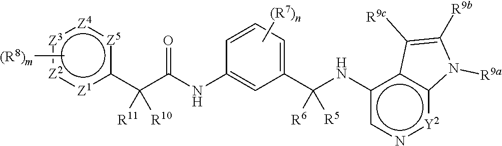 |
| (XVIII-B-2) | 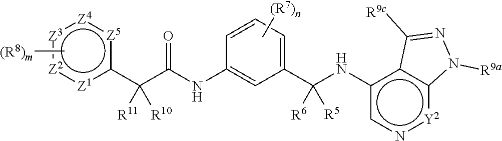 |
| (XX -2) | 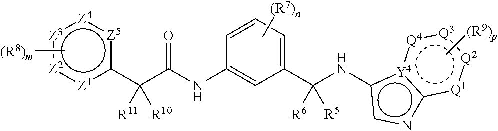 |

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein X is —NHC(O)NH—. In specific embodiments, compounds of Formulas (VIII-A), (VIII-B), (VIII-C), (VIII-D), (VIII-E), (VIII-F), (VIII-G), (VIII-H), (XI), (XVI), (XVIII-A), (XVIII-B), and (XX) are provided having the structure of any one of Formulas (VIII-A-3), (VIII-B-3), (VIII-C-3), (VIII-D-3), (VIII-E-3), (VIII-F-3), (VIII-G-3), (VIII-H-3), (XI-3), (XVI-3), (XVIII-A-3), (XVIII-B-3), or (XX-3) as shown in Table 3, below:

TABLE 3
EMBODIMENTS WHERE X IS —NHC(O)NH—

| Formula | Structure |
|---|---|
| (VIII-A-3) | 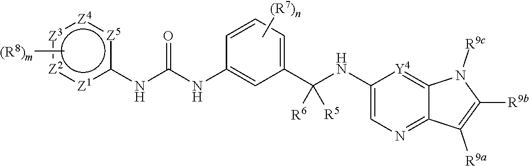 |
| (VIII-B-3) | 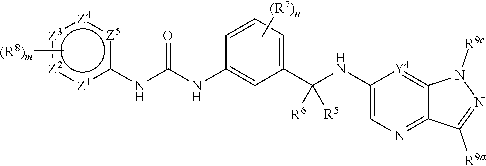 |
| (VIII-C-3) | 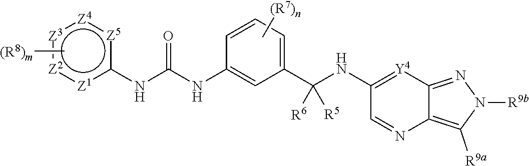 |
| (VIII-D-3) | 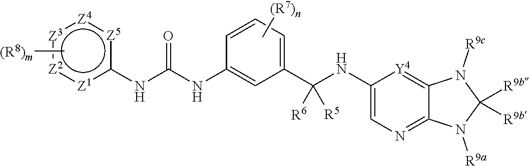 |
| (VIII-E-3) | 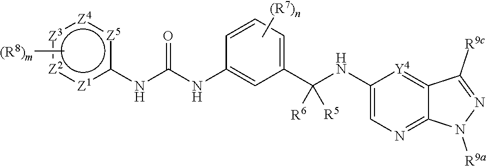 |
| (VIII-F-3) | 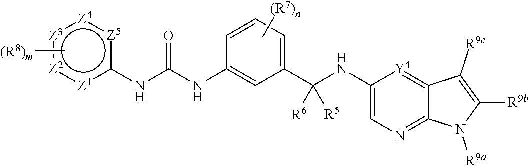 |
| (VIII-G-3) | 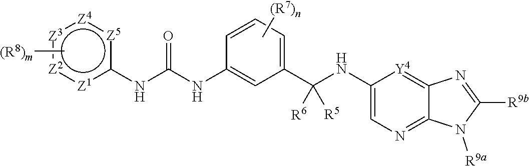 |
| (VIII-H-3) | 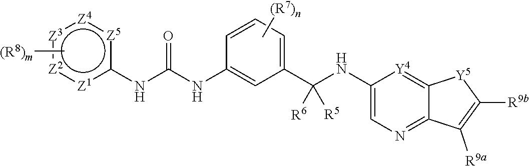 |
| (XI-3) | 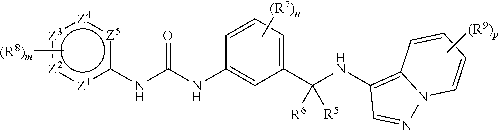 |
| (XVI-3) | 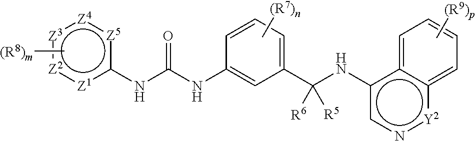 |
| (XVIII-A-3) | 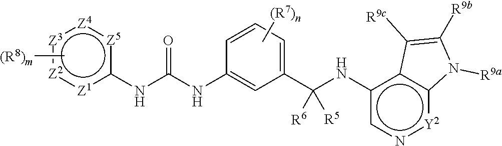 |
| (XVIII-B-3) | 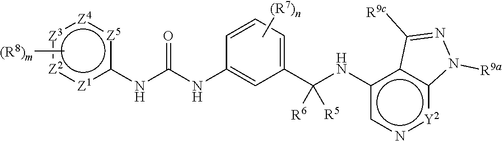 |
| (XX-3) | 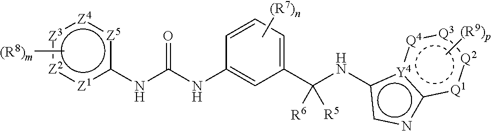 |

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein X is —NHC(O)—. In specific embodiments, compounds of Formulas (VIII-A), (VIII-B), (VIII-C), (VIII-D), (VIII-E), (VIII-F), (VIII-G), (VIII-H), (XI), (XVI), (XVIII-A), (XVIII-B), and (XX) are provided having the structure of any one of Formulas (VIII-A-4), (VIII-B-4), (VIII-C-4), (VIII-D-4), (VIII-E-4), (VIII-F-4), (VIII-G-4), (VIII-H-4), (XI-4), (XVI-4), (XVIII-A-4), (XVIII-B-4), or (XX-4) as shown in Table 4, below:

TABLE 4
EMBODIMENTS WHERE X IS —NHC(O)—

| Formula | Structure |
|---|---|
| (VIII-A-4) | 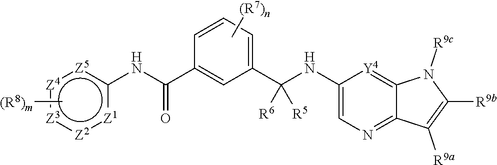 |
| (VIII-B-4) | 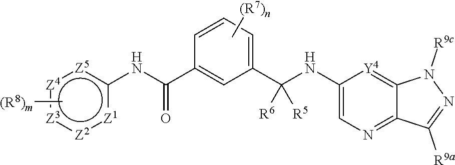 |
| (VIII-C-4) | 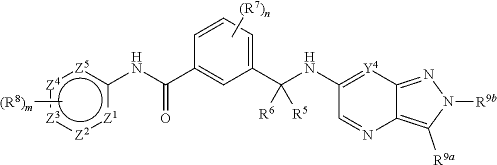 |
| (VIII-D-4) | 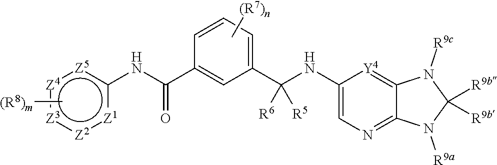 |
| (VIII-E-4) | 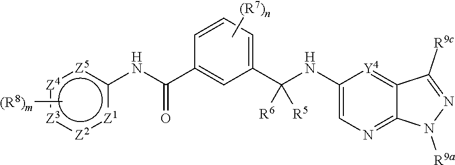 |
| (VIII-F-4) | 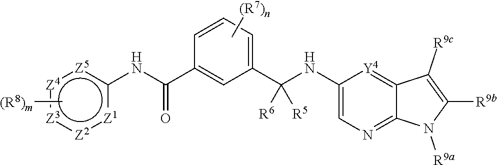 |
| (VIII-G-4) | 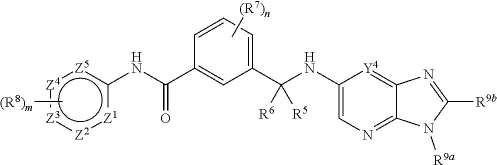 |
| (VIII-H-4) | 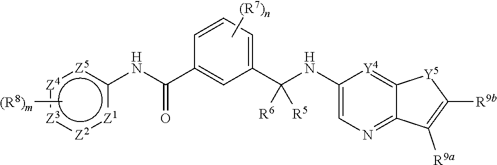 |
| (XI-4) | 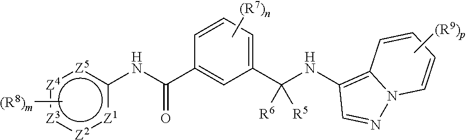 |
| (XVI-4) | 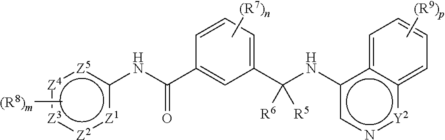 |
| (XVIII-A-4) | 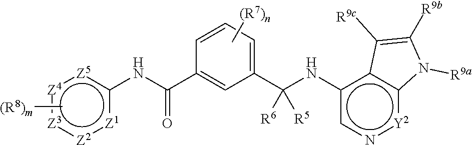 |
| (XVIII-B-4) | 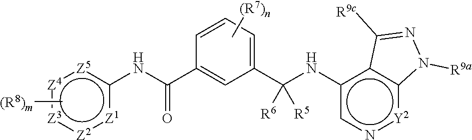 |
| (XX-4) | 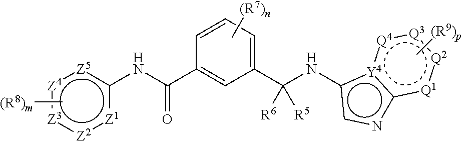 |

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein R⁵ is H and R⁶ is alkyl. In one embodiment, R⁶ is methyl.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII-A), (XVIII-B), (XIX), or (XX), as applicable, or a pharmaceutically acceptable isomer, racemate, hydrate, solvate, tautomer, isotope, or salt thereof, wherein n is 0. In one embodiment, n is 1 or 2.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein m is 0. In one embodiment, m is 1 or 2.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁸ is alkyl. In one embodiment, at least one R⁸ is methyl, ethyl, iso-propyl, n-propyl, or t-butyl.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁸ is alkyl substituted with halogen. In one embodiment, at least one R⁸ is difluoromethyl, trifluoromethyl, 2-fluoroethyl, or 2,2-difluoroethyl.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁸ is alkyl substituted with —OR^{a} and R^{a} is H or alkyl. In one embodiment, at least one R⁸ is hydroxymethyl, 2-hydroxyethyl, hydroxybutyl, or methoxymethyl.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁸ is carbocycle. In one embodiment, at least one R⁸ is cyclopropyl or cyclobutyl.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one of R⁸ is heterocycle.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁸ is —OR^{a}. In one embodiment, at least one R^{a} is alkyl. In one embodiment, at least one R^{a} is haloalkyl. In one embodiment, at least one R^{a} is carbocycle.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁸ is Nine′ in one embodiment, at least one R^{a} is H and at least one R^{b} is alkyl. In one embodiment, at least one R^{a} is H and at least one R^{b} is haloalkyl.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁸ is cyano.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁸ is halogen. In one embodiment, at least one R⁸ is Cl.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein p is 0. In one embodiment, p is 1 or 2.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁹ is halogen. In one embodiment, at least one of R⁹ is Cl or Br.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁹ is alkyl. In one embodiment, at least one of R⁹ is methyl or ethyl.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁹ is carbocycle. In one embodiment, at least one of R⁹ is phenyl.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁹ is heterocycle.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁹ is —OR^{a}. In one embodiment, R^{a} is, at each occurrence, independently H or alkyl.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁹ is optionally substituted with carbocycle or heterocycle.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein at least one R⁹ is optionally substituted with —OR^{a}. In one embodiment, R^{a} is, at each occurrence, independently H or alkyl.

In one embodiment, compounds are provided having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, or a pharmaceutically acceptable isomer, racemate, hydrate, solvate, tautomer, isotope, or salt thereof, wherein two R⁹ together form ═O.

Representative compounds having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), as applicable, include the compounds listed in Table 5 below, as well as pharmaceutically acceptable isomers, racemates, hydrates, solvates, isotopes, or salts thereof.

TABLE 5
REPRESENTATIVE COMPOUNDS

| Cmpd | | |
|---|---|---|
| No | Structure | Name |
| 1 | 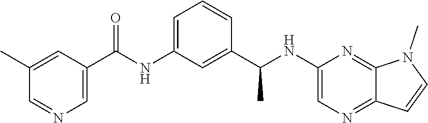 [SMILES: Cc1cncc(C(=O)Nc2cccc([C@H](C)Nc3cnc4ccn(C)c4n3)c2)c1] | (S)-5-methyl-N-(3-(1-((5- methyl-5H-pyrrolo[2,3-b] pyrazin-3-yl)amino)ethyl) phenyl)nicotinamide |
| 2 | 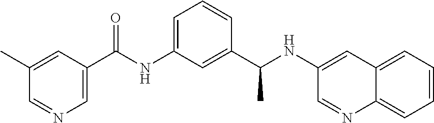 [SMILES: Cc1cncc(C(=O)Nc2cccc([C@H](C)Nc3cnc4ccccc4c3)c2)c1] | (S)-5-methyl-N-(3-(1-(quinolin- 3-ylamino)ethyl)phenyl) nicotinamide |
| 3 | 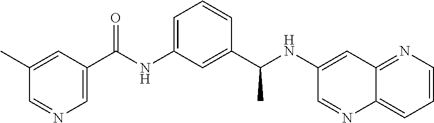 [SMILES: Cc1cncc(C(=O)Nc2cccc([C@H](C)Nc3cnc4cccnc4c3)c2)c1] | (S)-N-(3-(1-((1,5-naphthyridin- 3-yl)amino)ethyl)phenyl)-5- methylnicotinamide |
| 4 | 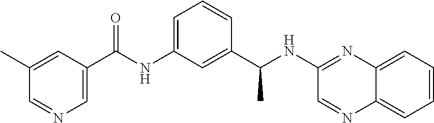 [SMILES: Cc1cncc(C(=O)Nc2cccc([C@H](C)Nc3cnc4ccccc4n3)c2)c1] | (S)-5-methyl-N-(3-(1- (quinoxalin-2-ylamino)ethyl) phenyl)nicotinamide |
| 5 | 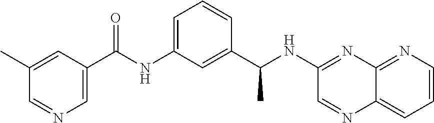 [SMILES: Cc1cncc(C(=O)Nc2cccc([C@H](C)Nc3cnc4cccnc4n3)c2)c1] | (S)-5-methyl-N-(3-(1-(pyrido [2,3-b]pyrazin-3-ylamino)ethyl) phenyl)nicotinamide |
| 6 | 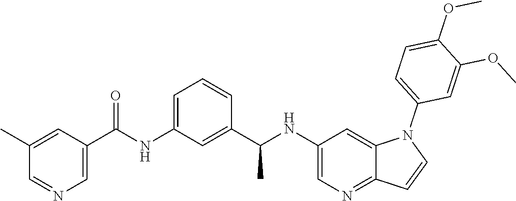 | (S)-N-(3-(1-((1-(3,4- dimethoxyphenyl)-1H-pyrrolo [3,2-b]pyridin-6-yl)amino)ethyl) phenyl)-5-methylnicotinamide |
| 7 | 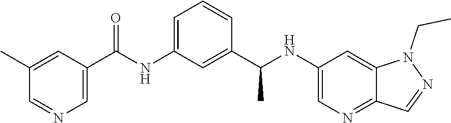 [SMILES: CCn1ncc2ncc(N[C@@H](C)c3cccc(NC(=O)c4cncc(C)c4)c3)cc21] | (S)-N-(3-(1-((1-ethyl-1H- pyrazolo[4,3]pyridin-6-yl) amino)ethyl)phenyl)-5- methylnicotinamide |
| 8 | 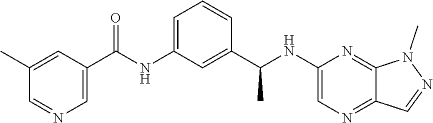 [SMILES: Cc1cncc(C(=O)Nc2cccc([C@H](C)Nc3cnc4cnn(C)c4n3)c2)c1] | (S)-5-methyl-N-(3-(1-((1- methyl-1H-pyrazolo[3,4-b] pyrazin-6-yl)amino)ethyl) phenyl)nicotinamide |
| 9 | 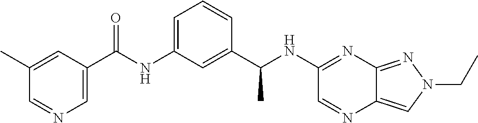 [SMILES: CCn1cc2ncc(N[C@@H](C)c3cccc(NC(=O)c4cncc(C)c4)c3)nc2n1] | (S)-N-(3-(1-((2-ethyl-2H- pyrazolo[3,4-b]pyrazin-6-yl) amino)ethyl)phenyl)-5- methylnicotinamide |
| 10 | 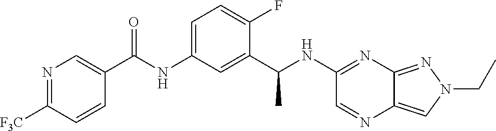 [SMILES: CCn1cc2ncc(N[C@@H](C)c3cc(NC(=O)c4ccc(C(F)(F)F)nc4)ccc3F)nc2n1] | (S)-N-(3-(1-((2-ethyl-2H- pyrazolo[3,4-b]pyrazin-6-yl) amino)ethyl)-4-fluorophenyl)-6- (trifluoromethyl)nicotinamide |
| 11 | 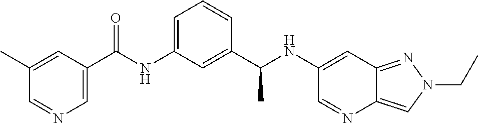 [SMILES: CCn1cc2ncc(N[C@@H](C)c3cccc(NC(=O)c4cncc(C)c4)c3)cc2n1] | (S)-N-(3-(1-((2-ethyl-2H- pyrazolo[4,3-b]pyridin-6-yl) amino)ethyl)phenyl)-5- methylnicotinamide |
| 12 | 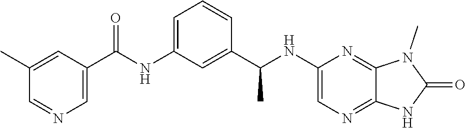 [SMILES: Cc1cncc(C(=O)Nc2cccc([C@H](C)Nc3cnc4[nH]c(=O)n(C)c4n3)c2)c1] | (S)-5-methyl-n-(3-(1-((3- methyl-2-oxo-2,3-dihydro-1H- imidazo[4,5-b]pyrazin-5-yl) amino)ethyl)phenyl) nicotinamide |
| 13 | 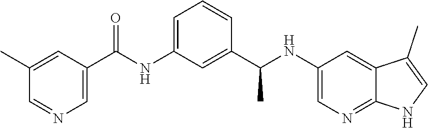 [SMILES: Cc1cncc(C(=O)Nc2cccc([C@H](C)Nc3cnc4[nH]cc(C)c4c3)c2)c1] | (S)-5-methyl-N-(3-(1-((3- methyl-1H-pyrrolo[2,3-b] pyridin-5-yl)amino)ethyl) phenyl)nicotinamide |
| 14 | 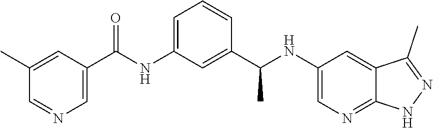 [SMILES: Cc1cncc(C(=O)Nc2cccc([C@H](C)Nc3cnc4[nH]nc(C)c4c3)c2)c1] | (S)-5-methyl-N-(3-(1-((3- methyl-1H-pyrazolo[3,4-b] pyridin-5-yl)amino)ethyl) phenyl)nicotinamide |
| 15 | 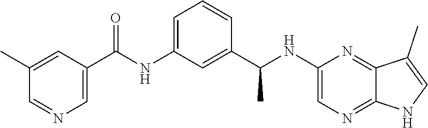 [SMILES: Cc1cncc(C(=O)Nc2cccc([C@H](C)Nc3cnc4[nH]cc(C)c4n3)c2)c1] | (S)-5-methyl-N-(3-(1-((7- methyl-5H-pyrrolo[2,3-b] pyrazin-2-yl)amino)ethyl) phenyl)nicotinamide |
| 16 | 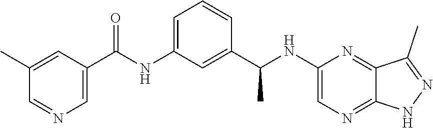 [SMILES: Cc1cncc(C(=O)Nc2cccc([C@H](C)Nc3cnc4[nH]nc(C)c4n3)c2)c1] | (S)-5-methyl-N-(3-(1-((3- methyl-1H-pyrazolo[3,4-b] pyrazin-5-yl)amino)ethyl) phenyl)nicotinamide |
| 17 | 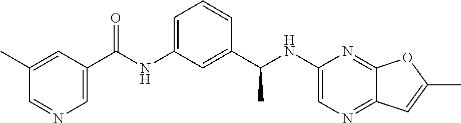 | (S)-5-methyl-N-(3-(1-((6- methylfuro[2,3-b]pyrazin-3-yl)- amino)ethyl)phenyl) nicotinamide |
| 18 | 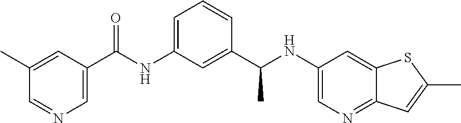 | (S)-5-methyl-N-(3-(1-((2- methylthieno[3,2-b]pyridin-6- yl)amino)ethyl)phenyl) nicotinamide |
| 19 | 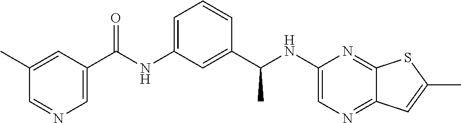 | (S)-5-methyl-N-(3-(1-((6- methylthieno[2,3-b]pyrazin-3- yl)amino)ethyl)phenyl) nicotinamide |
| 20 | 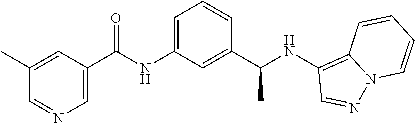 [SMILES: Cc1cncc(C(=O)Nc2cccc([C@H](C)Nc3cnn4ccccc34)c2)c1] | (S)-5-methyl-N-(3-(1- (pyrazolo[1,5-a]pyridin-3- ylamino)ethyl)phenyl) nicotinamide |
| 21 | 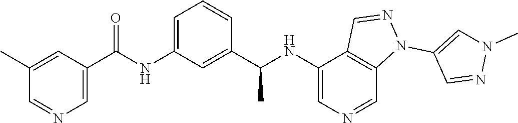 | (S)-5-methyl-N-(3-(1-((1-(1- methyl-1H-pyrazol-4-yl)-1H- pyrazolo[3,4-c]pyridin-4-yl) amino)ethyl)phenyl) nicotinamide |
| 22 | 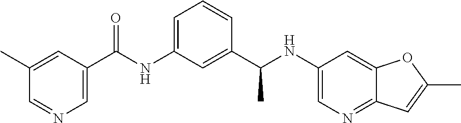 | (S)-5-methyl-N-(3-(1-((2- methylfuro[3,2-b]pyridin-6-yl) amino)ethyl)phenyl) nicotinamide |
| 23 | 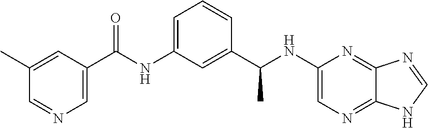 [SMILES: Cc1cncc(C(=O)Nc2cccc([C@H](C)Nc3cnc4[nH]cnc4n3)c2)c1] | (S)-N-(3-(1-((1H-imidazo[4,5-b] pyrazin-5-yl)amino)ethyl) phenyl)-5-methylnicotinamide |
| 24 | 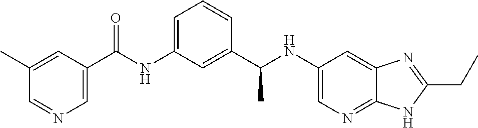 [SMILES: CCc1nc2cc(N[C@@H](C)c3cccc(NC(=O)c4cncc(C)c4)c3)cnc2[nH]1] | (S)-N-(3-(1-((2-ethyl-3H- imidazo[4,5-b]pyridin-6-yl) amino)ethyl)phenyl)-5- methylnicotinamide |
| 25 | 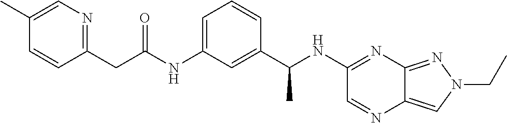 [SMILES: CCn1cc2ncc(N[C@@H](C)c3cccc(NC(=O)Cc4ccc(C)cn4)c3)nc2n1] | (S)-N-(3-(1-((2-ethyl-2H- pyrazolo[3,4-b]pyrazin-6-yl) amino)ethyl)phenyl)-2-(5- methylpyridin-2-yl)acetamide |
| 26 | 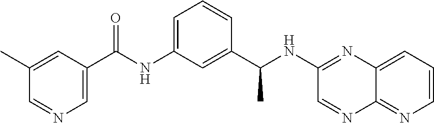 [SMILES: Cc1cncc(C(=O)Nc2cccc([C@H](C)Nc3cnc4ncccc4n3)c2)c1] | (S)-5-methyl-N-(3-(1- (pyrido[2,3-b]pyrazin-2- ylamino)ethyl)phenyl) nicotinamide |
| 27 | 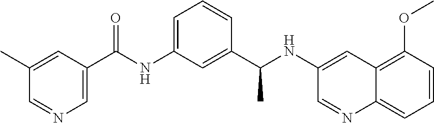 | (S)-N-(3-(1-((5- methoxyquinolin-3-yl)amino) ethyl)phenyl)-5- methylnicotinamide |
| 28 | 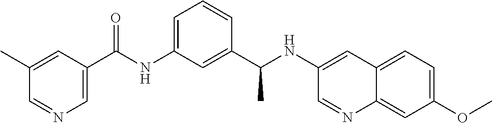 | (S)-N-(3-(1-((7- methoxyquinolin-3-yl)amino) ethyl)phenyl)-5- methylnicotinamide |
| 29 | 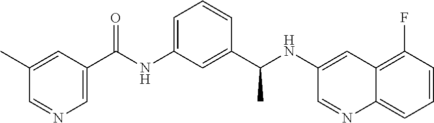 [SMILES: Cc1cncc(C(=O)Nc2cccc([C@H](C)Nc3cnc4cccc(F)c4c3)c2)c1] | (S)-N-(3-(1-((5-fluoroquinolin- 3-yl)amino)ethyl)phenyl)-5- methylnicotinamide |
| 30 | 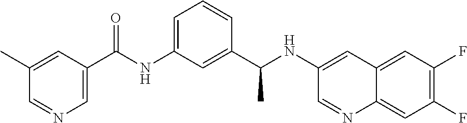 [SMILES: Cc1cncc(C(=O)Nc2cccc([C@H](C)Nc3cnc4cc(F)c(F)cc4c3)c2)c1] | (S)-N-(3-(1-((6,7- difluoroquinolin-3-yl)amino) ethyl)phenyl)-5- methylnicotinamide |
| 31 | 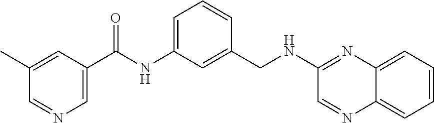 [SMILES: Cc1cncc(C(=O)Nc2cccc(CNc3cnc4ccccc4n3)c2)c1] | 5-methyl-N-(3-((quinoxalin-2- ylamino)methyl)phenyl) nicotinamide |
| 32 | 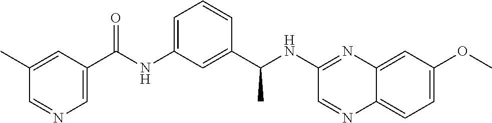 | (S)-N-(3-(1-((7- methoxyquinoxalin-2-yl)amino) ethyl)phenyl)-5- methylnicotinamide |
| 33 | 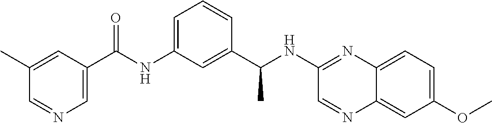 | (S)-N-(3-(1-((6- methoxyquinoxalin-2-yl)amino) ethyl)phenyl)-5- methylnicotinamide |
| 34 | 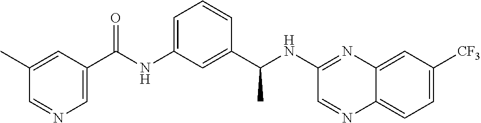 [SMILES: Cc1cncc(C(=O)Nc2cccc([C@H](C)Nc3cnc4ccc(C(F)(F)F)cc4n3)c2)c1] | (S)-5-methyl-N-(3-(1-((7- (trifluoromethyl)quinoxalin-2- yl)amino)ethyl)phenyl) nicotinamide |
| 35 | 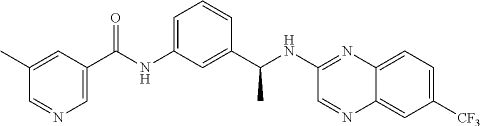 [SMILES: Cc1cncc(C(=O)Nc2cccc([C@H](C)Nc3cnc4cc(C(F)(F)F)ccc4n3)c2)c1] | (S)-5-methyl-N-(3-(1-((6- (trifluoromethyl)quinoxalin-2- yl)amino)ethyl)phenyl) nicotinamide |
| 36 | 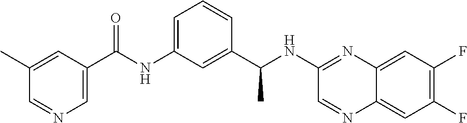 [SMILES: Cc1cncc(C(=O)Nc2cccc([C@H](C)Nc3cnc4cc(F)c(F)cc4n3)c2)c1] | (S)-N-(3-(1-((6,7- difluoroquinoxalin-2-yl)amino) ethyl)phenyl)-5- methylnicotinamide |
| 37 | 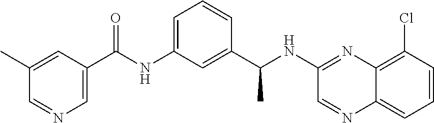 [SMILES: Cc1cncc(C(=O)Nc2cccc([C@H](C)Nc3cnc4cccc(Cl)c4n3)c2)c1] | (S)-N-(3-(1-((8- chloroquinoxalin-2-yl)amino) ethyl)phenyl)-5- methylnicotinamide |
| 38 | 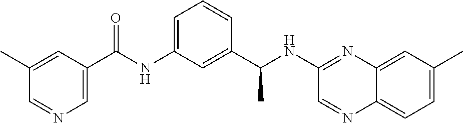 | (S)-5-methyl-N-(3-(1-((7- methylquinoxalin-2-yl)amino) ethyl)phenyl)nicotinamide |
| 39 | 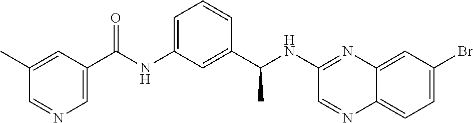 [SMILES: Cc1cncc(C(=O)Nc2cccc([C@H](C)Nc3cnc4ccc(Br)cc4n3)c2)c1] | (S)-N-(3-(1-((7- bromoquinoxalin-2-yl)amino) ethyl)phenyl)-5- methylnicotinamide |
| 40 | 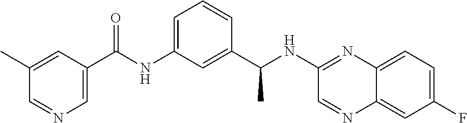 [SMILES: Cc1cncc(C(=O)Nc2cccc([C@H](C)Nc3cnc4cc(F)ccc4n3)c2)c1] | (S)-N-(3-(1-((6- fluoroquinoxalin-2-yl)amino) ethyl)phenyl)-5- methylnicotinamide |
| 41 | 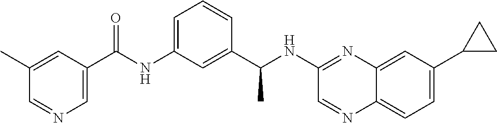 [SMILES: Cc1cncc(C(=O)Nc2cccc([C@H](C)Nc3cnc4ccc(C5CC5)cc4n3)c2)c1] | (S)-N-(3-(1-((7- cyclopropylquinoxalin-2-yl) amino)ethyl)phenyl)-5- methylnicotinamide |
| 42 | 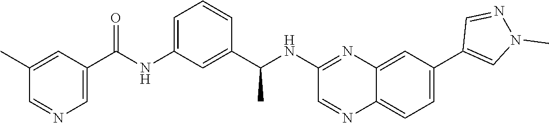 | (S)-5-methyl-N-(3-(1-((7-(1- methyl-1H-pyrazol-4-yl) quinoxalin-2-yl)amino)ethyl) phenyl)nicotinamide |
| 43 | 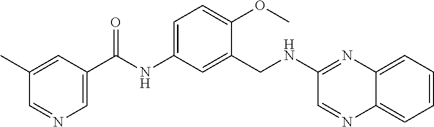 [SMILES: COc1ccc(NC(=O)c2cncc(C)c2)cc1CNc1cnc2ccccc2n1] | N-(4-methoxy-3-((quinoxalin-2- ylamino)methyl)phenyl)-5- methylnicotinamide |
| 44 | 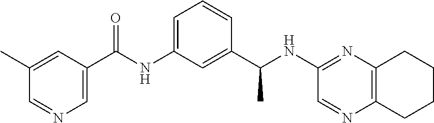 [SMILES: Cc1cncc(C(=O)Nc2cccc([C@H](C)Nc3cnc4c(n3)CCCC4)c2)c1] | (S)-5-methyl-N-(3-(1-((5,6,7,8- tetrahydroquinoxalin-2-yl) amino)ethyl)phenyl) nicotinamide |
| 45 | 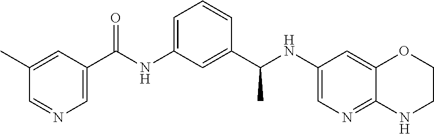 [SMILES: Cc1cncc(C(=O)Nc2cccc([C@H](C)Nc3cnc4c(c3)OCCN4)c2)c1] | (S)-N-(3-1-((3,4-dihydro-2H- pyrido[3,2-b][1,4]oxazin-7-yl) amino)ethyl)phenyl)-5- methylnicotinamide |
| 46 | 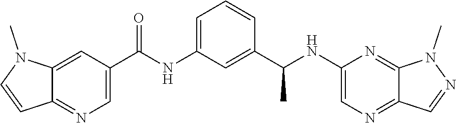 [SMILES: C[C@H](Nc1cnc2cnn(C)c2n1)c1cccc(NC(=O)c2cnc3ccn(C)c3c2)c1] | (S)-1-methyl-N-(3-(1-((1- methyl-1H-pyrazolo[3,4-b] pyrazin-6-yl)amino)ethyl) phenyl)-1H-pyrrolo[3,2-b] pyridine-6-carboxamide |
| 47 | 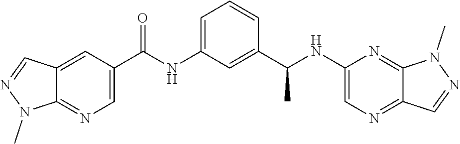 [SMILES: C[C@H](Nc1cnc2cnn(C)c2n1)c1cccc(NC(=O)c2cnc3c(cnn3C)c2)c1] | (S)-1-methyl-N-(3-(1-((1- methyl-1H-pyrazolo[3,4-b] pyrazin-6-yl)amino)ethyl) phenyl)-1H-pyrazolo[3,4-b] pyridine-6-carboxamide |
| 48 | 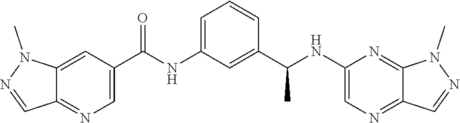 [SMILES: C[C@H](Nc1cnc2cnn(C)c2n1)c1cccc(NC(=O)c2cnc3cnn(C)c3c2)c1] | (S)-1-methyl-N-(3-(1-((1- methyl-1H-pyrazolo[3,4-b] pyrazin-6-yl)amino)ethyl) phenyl)-1H-pyrazolo[4,3-b] pyridine-6-carboxamide |
| 49 | 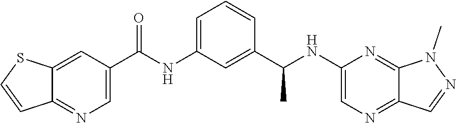 [SMILES: C[C@H](Nc1cnc2cnn(C)c2n1)c1cccc(NC(=O)c2cnc3ccsc3c2)c1] | (S)-N-(3-(1-((1-methyl-1H- pyrazolo[3,4-b]pyrazin-6-yl) amino)ethyl)phenyl) thieno[3,2-b]pyridinc-6- carboxamide |
| 50 | 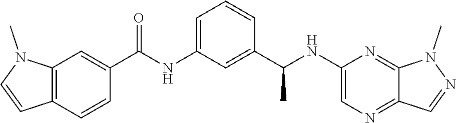 [SMILES: C[C@H](Nc1cnc2cnn(C)c2n1)c1cccc(NC(=O)c2ccc3ccn(C)c3c2)c1] | (S)-1-methyl-N-(3-(1-((1- methyl-1H-pyrazolo[3,4-b] pyrazin-6-yl)amino)ethyl) phenyl)-1H-indole-6- carboxamide |
| 51 | 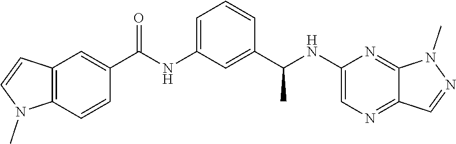 [SMILES: C[C@H](Nc1cnc2cnn(C)c2n1)c1cccc(NC(=O)c2ccc3c(ccn3C)c2)c1] | (S)-1-methyl-N-(3-(1-((1- methyl-1H-pyrazolo[3,4-b] pyrazin-6-yl)amino)ethyl) phenyl)-1H-indole-6- carboxamide |
| 52 | 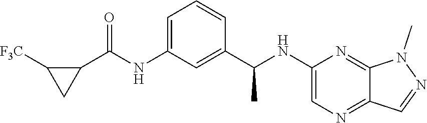 [SMILES: C[C@H](Nc1cnc2cnn(C)c2n1)c1cccc(NC(=O)C2CC2C(F)(F)F)c1] | N-(3-((S)-1-((1-methyl-1H- pyrazolo[3,4-b]pyrazin-6-yl) amino)ethyl)phenyl)-2- (trifluoromethyl)cyclopropane- 1-carboxamide |
| 53 | 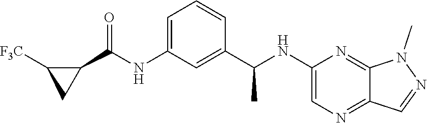 [SMILES: C[C@H](Nc1cnc2cnn(C)c2n1)c1cccc(NC(=O)[C@H]2C[C@H]2C(F)(F)F)c1] | (1S,2R)-N-(3-((S)-1-((1 methyl- 1H-pyrazolo[3,4-d]pyrazin-6-yl) amino)ethyl)phenyl)-2- (trifluoromethyl)cyclopropane- 1-carboxamide |
| 54 | 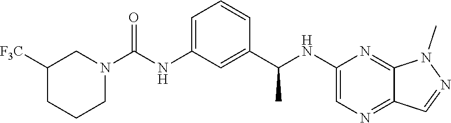 [SMILES: C[C@H](Nc1cnc2cnn(C)c2n1)c1cccc(NC(=O)N2CCCC(C(F)(F)F)C2)c1] | N-(3-((S)-1-((1-methyl-1H- pyrazolo[3,4-b]pyrazin-6-yl) amino)ethyl)phenyl)-3- (trifluoromethyl)piperidine-1- carboxamide |
| 55 | 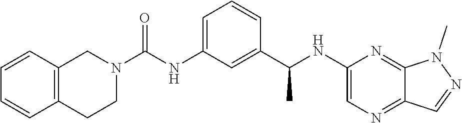 [SMILES: C[C@H](Nc1cnc2cnn(C)c2n1)c1cccc(NC(=O)N2CCc3ccccc3C2)c1] | (S)-N-(3-(1-((1-methyl-1H- pyrazolo[3,4-b]pyrazin-6-yl) amino)ethyl)phenyl)-3,4- dihydroisoquinoline-2(1H)- carboxamide |
| 56 | 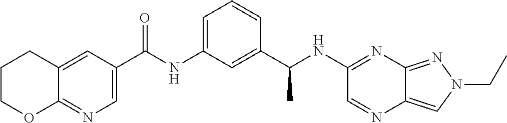 [SMILES: CCn1cc2ncc(N[C@@H](C)c3cccc(NC(=O)c4cnc5c(c4)CCCO5)c3)nc2n1] | (S)-N-(3-(1-((2-ethyl-2H- pyrazolo[3,4-b]pyrazin-6-yl) amino)ethyl)phenyl)-3,4- dihydro-2H-pyrano[2,3-b] pyridine-6-carboxamide |
| 57 | 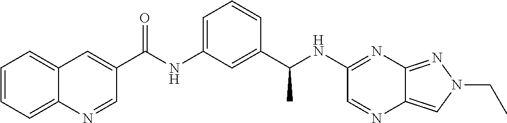 [SMILES: CCn1cc2ncc(N[C@@H](C)c3cccc(NC(=O)c4cnc5ccccc5c4)c3)nc2n1] | (S)-N-(3-(1-((2-ethyl-2H- pyrazolo[3,4-b]pyrazin-6-yl) amino)ethyl)phenyl)quinoline- 3-carboxamide |
| 58 | 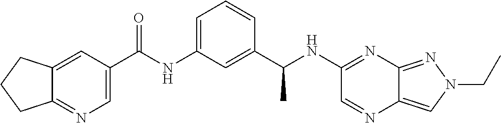 [SMILES: CCn1cc2ncc(N[C@@H](C)c3cccc(NC(=O)c4cnc5c(c4)CCC5)c3)nc2n1] | (S)-N-(3-(1-((2-ethyl-2H- pyrazolo[3,4-b]pyrazin-6-yl) amino)ethyl)phenyl)-6,7- dihydro-5H-cyclope3nta[6] pyridine-3-carboxamide |
| 59 | 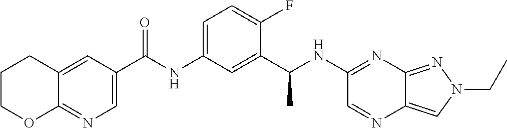 [SMILES: CCn1cc2ncc(N[C@@H](C)c3cc(NC(=O)c4cnc5c(c4)CCCO5)ccc3F)nc2n1] | (S)-N-(3-(1-((2-ethyl-2H- pyrazolo[3,4-b]pyrazin-6-yl) amino)ethyl)-4-fluorophenyl)- 3,4-dihydro-2H-pyrano[2,3-b] pyridine-6-carboxamide |
| 60 | 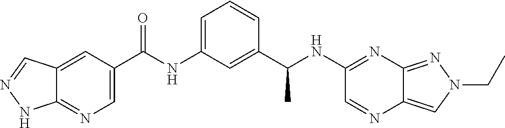 [SMILES: CCn1cc2ncc(N[C@@H](C)c3cccc(NC(=O)c4cnc5[nH]ncc5c4)c3)nc2n1] | (S)-N-(3-(1-((2-ethyl-2H- pyrazolo[3,4-b]pyrazin-6-yl) amino)ethyl)phenyl)-1H- pyrazolo[3,4-b]pyridine-5- carboxamide |
| 61 | 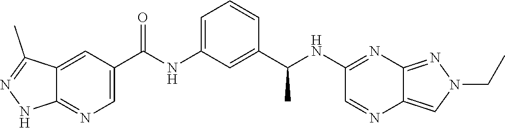 [SMILES: CCn1cc2ncc(N[C@@H](C)c3cccc(NC(=O)c4cnc5[nH]nc(C)c5c4)c3)nc2n1] | (S)-N-(3-(1-((2-ethyl-2H- pyrazolo[3,4-b]pyrazin-6-yl) amino)ethyl)phenyl)-3-methyl- 1H-pyrazolo[3,4-b]pyridine-5- carboxamide |
| 62 | 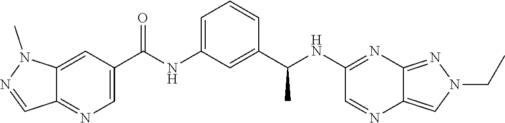 [SMILES: CCn1cc2ncc(N[C@@H](C)c3cccc(NC(=O)c4cnc5cnn(C)c5c4)c3)nc2n1] | (S)-N-(3-(1-((2-ethyl-2H- pyrazolo[3,4-b]pyrazin-6-yl) amino)ethyl)phenyl)-1-methyl- 1H-pyrazolo[4,3-b]pyridine-6- carboxamide |
| 63 | 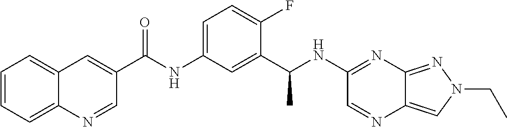 [SMILES: CCn1cc2ncc(N[C@@H](C)c3cc(NC(=O)c4cnc5ccccc5c4)ccc3F)nc2n1] | (S)-N-(3-(1-((2-ethyl-2H- pyrazolo[3,4-b]pyrazin-6-yl) amino)ethyl)-4-fluorophenyl) quinoline-3-carboxamide |
| 64 | 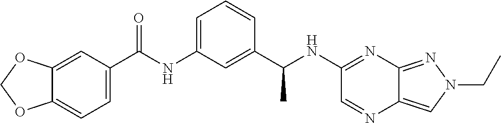 [SMILES: CCn1cc2ncc(N[C@@H](C)c3cccc(NC(=O)c4ccc5c(c4)OCO5)c3)nc2n1] | (S)-N-(3-(1-((2-ethyl-2H- pyrazolo[3,4-b]pyrazin-6-yl) amino)ethyl)phenyl) benzo[d][1,3]dioxole-5- carboxamide |
| 65 | 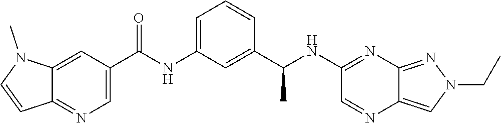 [SMILES: CCn1cc2ncc(N[C@@H](C)c3cccc(NC(=O)c4cnc5ccn(C)c5c4)c3)nc2n1] | (S)-N-(3-(1-((2-ethyl-2H- pyrazolo[3,4-b]pyrazin-6-yl) amino)ethyl)phenyl)-1-methyl- 1H-pyrrolo[3,2-b]pyridine-6- carboxamide |
| 66 | 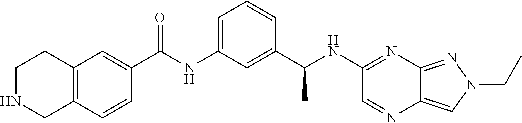 [SMILES: CCn1cc2ncc(N[C@@H](C)c3cccc(NC(=O)c4ccc5c(c4)CCNC5)c3)nc2n1] | (S)-N-(3-(1-((2-ethyl-2H- pyrazolo[3,4-b]pyrazin-6-yl) amino)ethyl)phenyl)-1,2,3,4- tetrahydroisoquinoline-6- carboxamide |
| 67 | 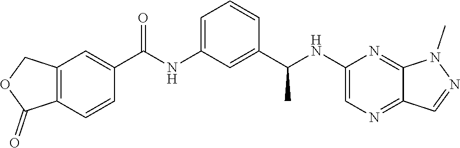 [SMILES: C[C@H](Nc1cnc2cnn(C)c2n1)c1cccc(NC(=O)c2ccc3c(c2)COC3=O)c1] | (S)-N-(3-(1-((1-methyl-1H- pyrazolo[3,4-b]pyrazin-6-yl) amino)ethyl)phenyl)-1-oxo-1,3- dihydroisobenzofuran-5- carboxamide |
| 68 | 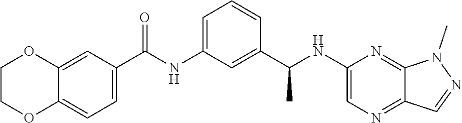 [SMILES: C[C@H](Nc1cnc2cnn(C)c2n1)c1cccc(NC(=O)c2ccc3c(c2)OCCO3)c1] | (S)-N-(3-(1-((1-methyl-1H- pyrazolo[3,4-b]pyrazin-6-yl) amino)ethyl)phenyl)-2,3- dihydrobenzo[b][1,4]dioxine-6- carboxamide |
| 69 | 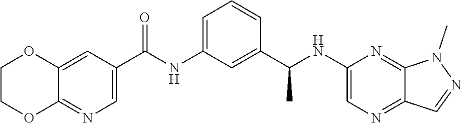 [SMILES: C[C@H](Nc1cnc2cnn(C)c2n1)c1cccc(NC(=O)c2cnc3c(c2)OCCO3)c1] | (S)-N-(3-(1-((1-methyl-1H- pyrazolo[3,4-b]pyrazin-6-yl) amino)ethyl)phenyl)-2,3- dihydro-[1,4]dioxino[2,3-b] pyridine-7-carboxamide |
| 70 | 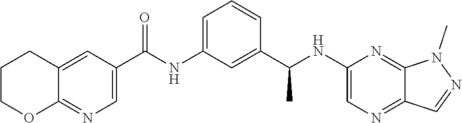 [SMILES: C[C@H](Nc1cnc2cnn(C)c2n1)c1cccc(NC(=O)c2cnc3c(c2)CCCO3)c1] | (S)-N-(3-(1-((1-methyl-1H- pyrazolo[3,4-b]pyrazin-6-yl) amino)ethyl)phenyl)-3,4- dihydro-2H-pyrano[2,3-b] pyridine-6-carboxamide |
| 71 | 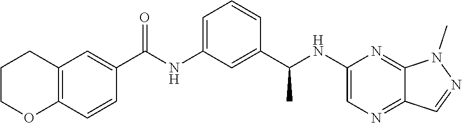 [SMILES: C[C@H](Nc1cnc2cnn(C)c2n1)c1cccc(NC(=O)c2ccc3c(c2)CCCO3)c1] | (S)-N-(3-(1-((1-methyl-1H- pyrazolo[3,4-b]pyrazin-6-yl) amino)ethyl)phenyl)chromane- 6-carboxamide |
| 72 | 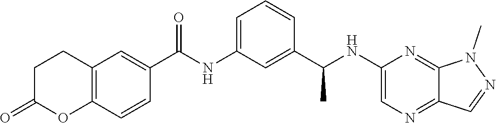 [SMILES: C[C@H](Nc1cnc2cnn(C)c2n1)c1cccc(NC(=O)c2ccc3c(c2)CCC(=O)O3)c1] | (S)-N-(3-(1-((1-methyl-1H- pyrazolo[3,4-b]pyrazin-6-yl) amino)ethyl)phenyl)-2- oxochromane-6-carboxamide |
| 73 | 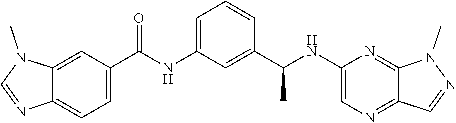 [SMILES: C[C@H](Nc1cnc2cnn(C)c2n1)c1cccc(NC(=O)c2ccc3ncn(C)c3c2)c1] | (S)-1-methyl-N-(3-(1-((1- methyl-1H-pyrazolo[3,4-b] pyrazin-6-yl)amino)ethyl) phenyl)-1H-benzo[d]imidazole- 6-carboxamide |
| 74 | 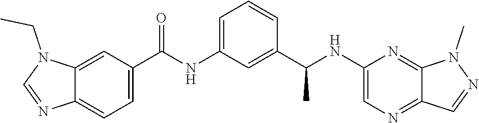 [SMILES: CCn1cnc2ccc(C(=O)Nc3cccc([C@H](C)Nc4cnc5cnn(C)c5n4)c3)cc21] | (S)-1-ethyl-N-(3-(1-((1-methyl- 1H-pyrazolo[3,4-b]pyrazin-6-yl) amino)ethyl)phenyl)-1H- benzo[d]imidazole-6- carboxamide |
| 75 | 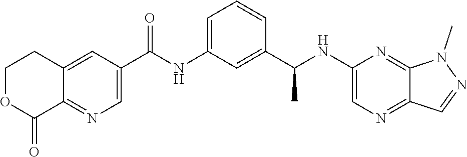 [SMILES: C[C@H](Nc1cnc2cnn(C)c2n1)c1cccc(NC(=O)c2cnc3c(c2)CCOC3=O)c1] | (S)-N-(3-(1-((1-methyl-1H- pyrazolo[3,4-b]pyrazin-6-yl) amino)ethyl)phenyl)-8-oxo-5,8- dihydro-6H-pyrano[3,4-b] pyridine-3-carboxamide |
| 76 | 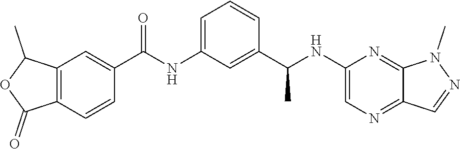 [SMILES: CC1OC(=O)c2ccc(C(=O)Nc3cccc([C@H](C)Nc4cnc5cnn(C)c5n4)c3)cc21] | 3-methyl-N-(3-((S)-1-((1- methyl-N-pyrazolo[3,4-b] pyrazin-6-yl)amino)ethyl) phenyl)-1-oxo-1,3- dihydroisobenzofuran-5- carboxamide |
| 77 | 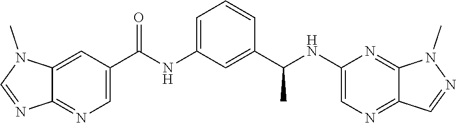 [SMILES: C[C@H](Nc1cnc2cnn(C)c2n1)c1cccc(NC(=O)c2cnc3ncn(C)c3c2)c1] | (S)-1-methyl-N-(3-(1-((1- methyl-1H-pyrazolo[3,4-b] pyrazin-6-yl)amino)ethyl) phenyl)-1H-imidazo[4,5-b] pyridine-6-carboxamide |
| 78 | 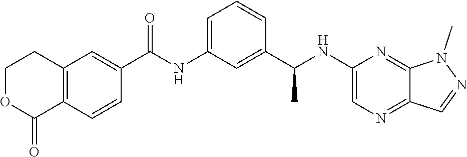 [SMILES: C[C@H](Nc1cnc2cnn(C)c2n1)c1cccc(NC(=O)c2ccc3c(c2)CCOC3=O)c1] | (S)-N-(3-(1-((1-methyl-1H- pyrazolo[3,4-b]pyrazin-6-yl) amino)ethyl)phenyl)-1- oxoisochromane-6-carboxamide |
| 79 | 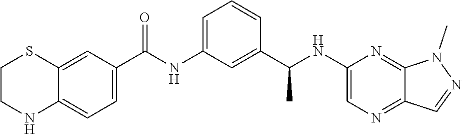 [SMILES: C[C@H](Nc1cnc2cnn(C)c2n1)c1cccc(NC(=O)c2ccc3c(c2)SCCN3)c1] | (S)-N-(3-(1-((1-methyl-1H- pyrazolo[3,4-d]pyrazin-6-yl) amino)ethyl)phenyl)-3,4- dihydro-2H-benzo[b] [1,4]thiazine-7-carboxamide |
| 80 | 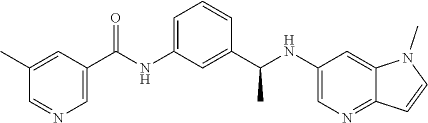 [SMILES: Cc1cncc(C(=O)Nc2cccc([C@H](C)Nc3cnc4ccn(C)c4c3)c2)c1] | (S)-5-methyl-N-(3-(1-((1- methyl-1H-pyrrolo[3,2-b] pyridin-6-yl)amino)ethyl) phenyl)nicotinamide |
| 81 | 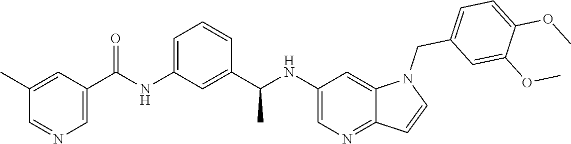 | (S)-N-(3-(1-((1-(3,4- dimethoxybenzyl)-1H-pyrrolo [3,2-b]pyridin-6-yl)amino)ethyl) phenyl)-5-methylnicotinamide |
| 82 | 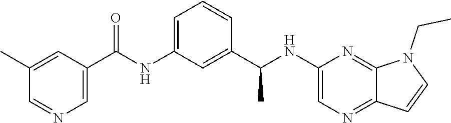 [SMILES: CCn1ccc2ncc(N[C@@H](C)c3cccc(NC(=O)c4cncc(C)c4)c3)nc21] | (S)-N-(3-(1-((5-ethyl-5H- pyrrolo[2,3-b]pyrazin-3-yl) amino)ethyl)phenyl)-5- methylnicotinamide |
| 83 | 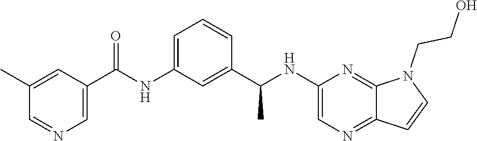 [SMILES: Cc1cncc(C(=O)Nc2cccc([C@H](C)Nc3cnc4ccn(CCO)c4n3)c2)c1] | (S)-N-(3-(1-((5-(2- hydroxyethyl)-5H-pyrrolo[2,3- 6]pyrazin-3-yl)amino)ethyl) phenyl)-5-methylnicotinamide |
| 84 | 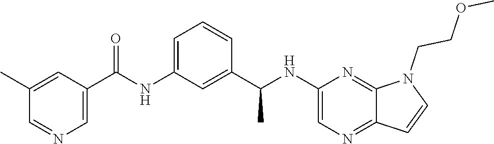 | (S)-N-(3-(1-((5-(2- methoxyethyl)-5H-pyrrolo [2,3-b]pyrazin-3-yl)amino) ethyl)phenyl)-5- methylnicotinamide |
| 85 | 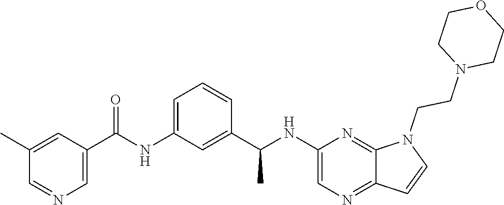 [SMILES: Cc1cncc(C(=O)Nc2cccc([C@H](C)Nc3cnc4ccn(CCN5CCOCC5)c4n3)c2)c1] | (S)-5-methyl-N-(3-(1-((5-(2- morpholinoethyl)-5H-pyrrolo [2,3-b]pyrazin-3-yl)amino) ethyl)phenyl)nicotinamide |
| 86 | 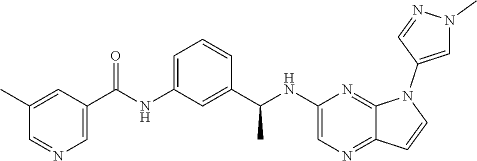 | (S)-5-methyl-N-(3-(1-((5-(1- methyl-1H-pyrazol-4-yl)-5H- pyrrolo[2,3-b]pyrazin-3-yl) amino)ethyl)phenyl) nicotinamide |
| 87 | 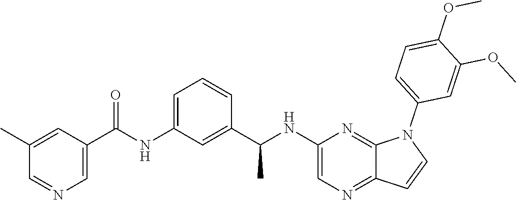 | (S)-N-(3-(1-((5-(3,4- dimethoxyphenyl)-5H-pyrrolo [2,3-b]\|pyrazin-3-yl)amino) ethyl)phenyl)-5- methylnicotinamide |
| 88 | 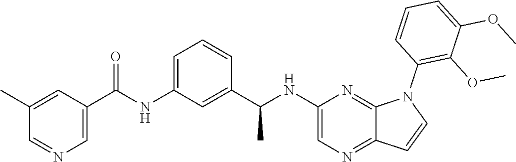 | (S)-N-(3-(1-((5-(3,4- dimethoxybenzyl)-5H-pyrrolo [2,3-b]pyrazin-3-yl)amino) ethyl)phenyl)-5- methylnicotinamide |
| 89 | 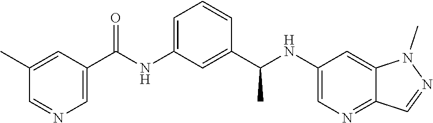 [SMILES: Cc1cncc(C(=O)Nc2cccc([C@H](C)Nc3cnc4cnn(C)c4c3)c2)c1] | (S)-5-methyl-N-(3-(1-((1- methyl-1H-pyrazolo[4,3-b] pyridin-6-yl)amino)ethyl) phenyl)nicotinamide |
| 90 | 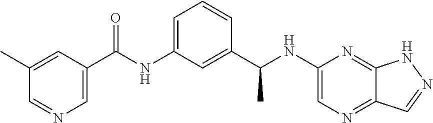 [SMILES: Cc1cncc(C(=O)Nc2cccc([C@H](C)Nc3cnc4cn[nH]c4n3)c2)c1] | (S)-N-(3-(1-((1H-pyrazolo[3,4- b]pyrazin-6-yl)amino)ethyl) phenyl)-5-methylnicotinamide |
| 91 | 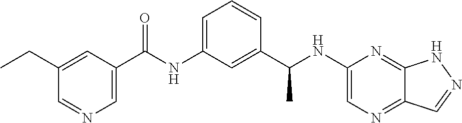 [SMILES: CCc1cncc(C(=O)Nc2cccc([C@H](C)Nc3cnc4cn[nH]c4n3)c2)c1] | (S)-N-(3-(1-((1H-pyrazolo[3,4- b]pyrazin-6-yl)amino)ethyl) phenyl)-5-ethylnicotinamide |
| 92 | 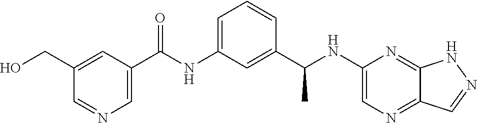 [SMILES: C[C@H](Nc1cnc2cn[nH]c2n1)c1cccc(NC(=O)c2cncc(CO)c2)c1] | (S)-N-(3-(1-((1H-pyrazolo[3,4- b]pyrazin-6-yl)amino)ethyl) phenyl)-5-(hydroxymethyl) nicotinamide |
| 93 | 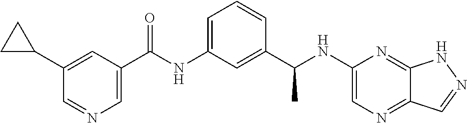 [SMILES: C[C@H](Nc1cnc2cn[nH]c2n1)c1cccc(NC(=O)c2cncc(C3CC3)c2)c1] | (S)-N-(3-(1-((1H-pyrazolo[3,4- b]pyrazin-6-yl)amino)ethyl) phenyl)-5- cyclopropylnicotinamide |
| 94 | 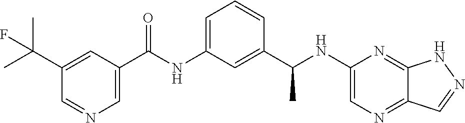 [SMILES: C[C@H](Nc1cnc2cn[nH]c2n1)c1cccc(NC(=O)c2cncc(C(C)(C)F)c2)c1] | (S)-N-(3-(1-((1H-pyrazolo[3,4- b]pyrazin-6-yl)amino)ethyl) phenyl)-5-(2-fluoropropan-2-yl) nicotinamide |
| 95 | 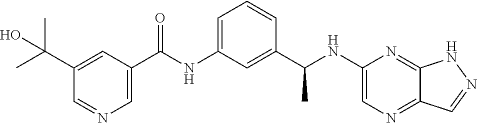 [SMILES: C[C@H](Nc1cnc2cn[nH]c2n1)c1cccc(NC(=O)c2cncc(C(C)(C)O)c2)c1] | (S)-N-(3-(1-((1H-pyrazolo[3,4- 6]pyrazin-6-yl)amino)ethyl) phenyl)-5-(2-hydroxypropan-2- yl)nicotinamide |
| 96 | 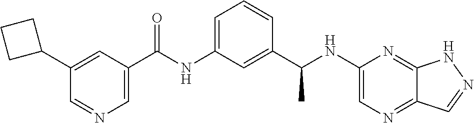 [SMILES: C[C@H](Nc1cnc2cn[nH]c2n1)c1cccc(NC(=O)c2cncc(C3CCC3)c2)c1] | (S)-N-(3-(1-((1H-pyrazolo[3,4- b]pyrazin-6-yl)amino)ethyl) phenyl)-5- cyclobutylnicotinamide |
| 97 | 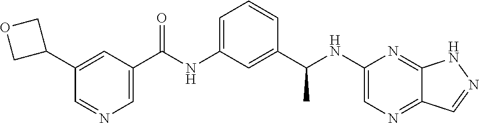 [SMILES: C[C@H](Nc1cnc2cn[nH]c2n1)c1cccc(NC(=O)c2cncc(C3COC3)c2)c1] | (S)-N-(3-(1-((1H-pyrazolo[3,4- b]pyrazin-6-yl)amino)ethyl) phenyl)-5-(oxetan-3-yl) nicotinamide |
| 98 | 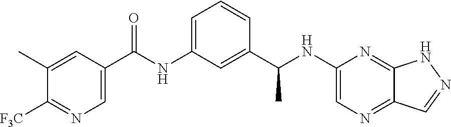 [SMILES: Cc1cc(C(=O)Nc2cccc([C@H](C)Nc3cnc4cn[nH]c4n3)c2)cnc1C(F)(F)F] | (S)-N-(3-(1-((1H-pyrazolo[3,4- b]pyrazin-6-yl)amino)ethyl) phenyl)-5-methyl-6- (trifluoromethyl)nicotinamide |
| 99 | 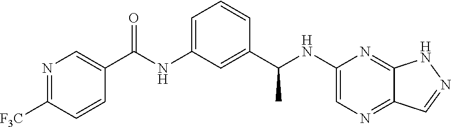 [SMILES: C[C@H](Nc1cnc2cn[nH]c2n1)c1cccc(NC(=O)c2ccc(C(F)(F)F)nc2)c1] | (S)-N-(3-(1-((1H-pyrazolo[3,4 b]pyrazin-6-yl)amino)ethyl) phenyl)-6-(trifluoromethyl) nicotinamide |
| 100 | 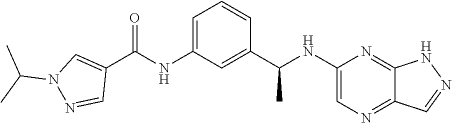 [SMILES: CC(C)n1cc(C(=O)Nc2cccc([C@H](C)Nc3cnc4cn[nH]c4n3)c2)cn1] | (S)-N-(3-(1-((1H-pyrazolo[3,4- b]pyrazin-6-yl)amino)ethyl) phenyl)-1-isopropyl-1H- pyrazole-4-carboxamide |
| 101 | 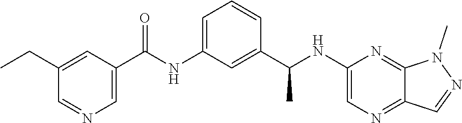 [SMILES: CCc1cncc(C(=O)Nc2cccc([C@H](C)Nc3cnc4cnn(C)c4n3)c2)c1] | (S)-N-ethyl-N-(3-(1-((1-methyl- 1H-pyrazolo[3,4-b]pyrazin-6-yl) amino)ethyl)phenyl) nicotinamide |
| 102 | 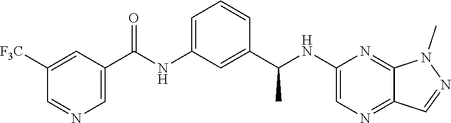 [SMILES: C[C@H](Nc1cnc2cnn(C)c2n1)c1cccc(NC(=O)c2cncc(C(F)(F)F)c2)c1] | (S)-N-(3-(1-((1-methyl-1H- pyrazolo[3,4-b]pyrazin-6-yl) amino)ethyl)phenyl)-5- (trifluoromethyl)nicotinamide |
| 103 | 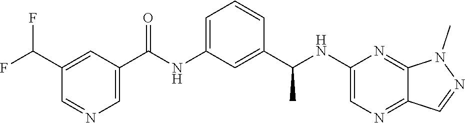 [SMILES: C[C@H](Nc1cnc2cnn(C)c2n1)c1cccc(NC(=O)c2cncc(C(F)F)c2)c1] | (S)-5-(difluoromethyl)-N-(3-(1- ((1-methyl-1H-pyrazolo[3,4-b] pyrazin-6-yl)amino)ethyl) phenyl)nicotinamide |
| 104 | 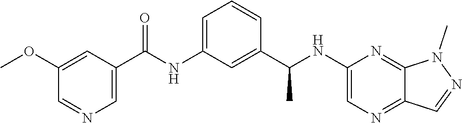 [SMILES: COc1cncc(C(=O)Nc2cccc([C@H](C)Nc3cnc4cnn(C)c4n3)c2)c1] | (S)-5-methoxy-N-(3-(1-((1- methyl-1H-pyrazolo[3,4-b] pyrazin-6-yl)amino)ethyl) phenyl)nicotinamide |
| 105 | 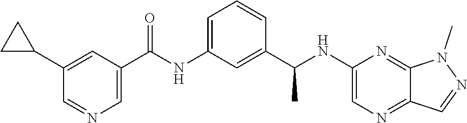 [SMILES: C[C@H](Nc1cnc2cnn(C)c2n1)c1cccc(NC(=O)c2cncc(C3CC3)c2)c1] | (S)-5-cyclopropyl-N-(3-(1-((1- methyl-1H-pyrazolo[3,4-b] pyrazin-6-yl)amino)ethyl) phenyl)nicotinamide |
| 106 | 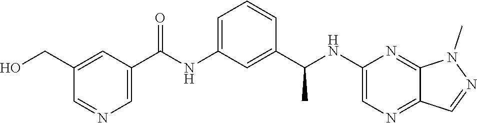 [SMILES: C[C@H](Nc1cnc2cnn(C)c2n1)c1cccc(NC(=O)c2cncc(CO)c2)c1] | (S)-5-(hydroxymethyl)-N-(3-(1- ((1-methyl-1H-pyrazolo[3,4-b] pyrazin-6-yl)amino)ethyl) phenyl)nicotinamide |
| 107 | 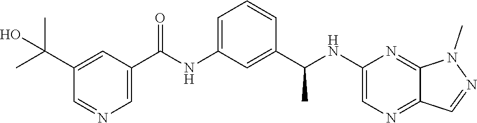 [SMILES: C[C@H](Nc1cnc2cnn(C)c2n1)c1cccc(NC(=O)c2cncc(C(C)(C)O)c2)c1] | (S)-5-(2-hydroxypropan-2-yl)- N-(3-(1-((1-methyl-1H- pyrazolo[3,4-b]pyrazin-6-yl) amino)ethyl)phenyl) nicotinamide |
| 108 | 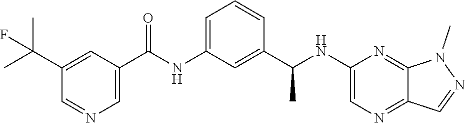 [SMILES: C[C@H](Nc1cnc2cnn(C)c2n1)c1cccc(NC(=O)c2cncc(C(C)(C)F)c2)c1] | (S)-5-(2-fluoropropan-2-yl)-N- (3-(1-((1-methyl-1H- pyrazolo[3,4-b]pyrazin-6-yl) amino)ethyl)phenyl) nicotinamide |
| 109 | 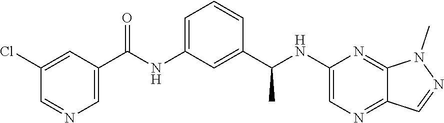 [SMILES: C[C@H](Nc1cnc2cnn(C)c2n1)c1cccc(NC(=O)c2cncc(Cl)c2)c1] | (S)-5-chloro-N-(3-(1-((1- methyl-1H-pyrazolo[3,4-b] pyrazin-6-yl)amino)ethyl) phenyl)nicotinamide |
| 110 | 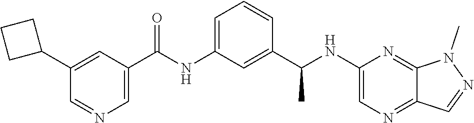 [SMILES: C[C@H](Nc1cnc2cnn(C)c2n1)c1cccc(NC(=O)c2cncc(C3CCC3)c2)c1] | (S)-5-cyclobutyl-N-(3-(1-((1- methyl-1H-pyrazolo[3,4-b] pyrazin-6-yl)amino)ethyl) phenyl)nicotinamide |
| 111 | 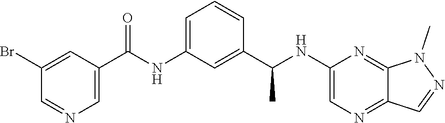 [SMILES: C[C@H](Nc1cnc2cnn(C)c2n1)c1cccc(NC(=O)c2cncc(Br)c2)c1] | (S)-5-bromo-N-(3-(1-((1- methyl-1H-pyrazolo[3,4-b] pyrazin-6-yl)amino)ethyl) phenyl)nicotinamide |
| 112 | 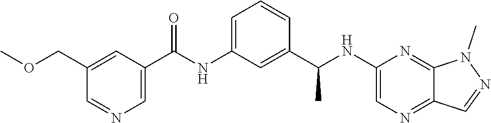 [SMILES: COCc1cncc(C(=O)Nc2cccc([C@H](C)Nc3cnc4cnn(C)c4n3)c2)c1] | (S)-5-(methoxymethyl)-N-(3-(1- ((1-methyl-1H-pyrazolo[3,4-b] pyrazin-6-yl)amino)ethyl) phenyl)nicotinamide |
| 113 | 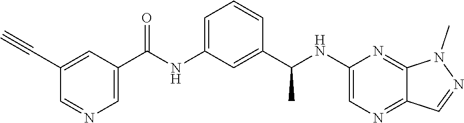 [SMILES: C#Cc1cncc(C(=O)Nc2cccc([C@H](C)Nc3cnc4cnn(C)c4n3)c2)c1] | (S)-5-ethynyl-N-(3-(1-((1- methyl-1H-pyrazolo[3,4-b] pyrazin-6-yl)amino)ethyl) phenyl)nicotinamide |
| 114 | 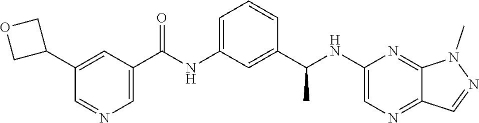 [SMILES: C[C@H](Nc1cnc2cnn(C)c2n1)c1cccc(NC(=O)c2cncc(C3COC3)c2)c1] | (S)-N-(3-(1-((1-methyl-1H- pyrazolo[3,4-b]pyrazin-6-yl) amino)ethyl)phenyl)-5-(oxetan- 3-yl)nicotinamide |
| 115 | 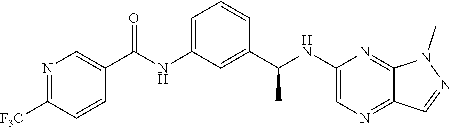 [SMILES: C[C@H](Nc1cnc2cnn(C)c2n1)c1cccc(NC(=O)c2ccc(C(F)(F)F)nc2)c1] | (S)-N-(3-(1-((1-methyl-1H- pyrazolo[3,4-b]pyrazin-6-yl) amino)ethyl)phenyl)-6- (trifluoromethyl)nicotinamide |
| 116 | 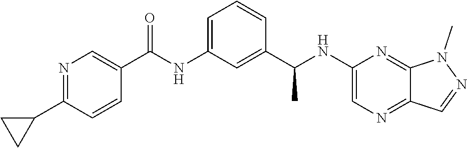 [SMILES: C[C@H](Nc1cnc2cnn(C)c2n1)c1cccc(NC(=O)c2ccc(C3CC3)nc2)c1] | (S)-6-cyclopropyl-N-(3-(1-((1- methyl-1H-pyrazolo[3,4-b] pyrazin-6-yl)amino)ethyl) phenyl)nicotinamide |
| 117 | 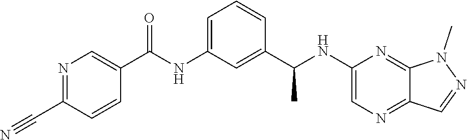 [SMILES: C[C@H](Nc1cnc2cnn(C)c2n1)c1cccc(NC(=O)c2ccc(C#N)nc2)c1] | (S)-6-cyano-N-(3-(1-((1-methyl- 1H-pyrazolo[3,4-b]pyrazin-6-yl) amino)ethyl)phenyl) nicotinamide |
| 118 | 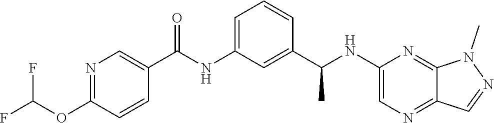 [SMILES: C[C@H](Nc1cnc2cnn(C)c2n1)c1cccc(NC(=O)c2ccc(OC(F)F)nc2)c1] | (S)-6-(difluoromethoxy)-N-(3- (1-((1-methyl-1H-pyrazolo[3,4- 6]pyrazin-6-yl)amino)ethyl) phenyl)nicotinamide |
| 119 | 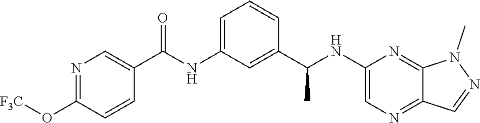 [SMILES: C[C@H](Nc1cnc2cnn(C)c2n1)c1cccc(NC(=O)c2ccc(OC(F)(F)F)nc2)c1] | (S)-N-(3-(1-((1-methyl-1H- pyrazolo[3,4-b]pyrazin-6-yl) amino)ethyl)phenyl)-6- (trifluoromethoxy)nicotinamide |
| 120 | 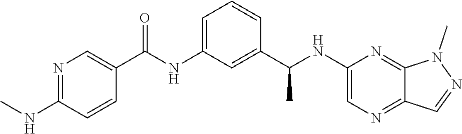 [SMILES: CNc1ccc(C(=O)Nc2cccc([C@H](C)Nc3cnc4cnn(C)c4n3)c2)cn1] | (S)-N-(3-(1-((1-methyl-1H- pyrazolo[3,4-b]pyrazin-6-yl) amino)ethyl)phenyl)-6- (methylamino)nicotinamide |
| 121 | 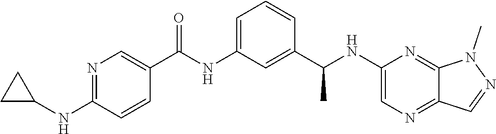 [SMILES: C[C@H](Nc1cnc2cnn(C)c2n1)c1cccc(NC(=O)c2ccc(NC3CC3)nc2)c1] | (S)-6-(cyclopropylamino)-N-(3- (1-((1-methyl-1H-pyrazolo[3,4- 6]pyrazin-6-yl)amino)ethyl) phenyl)nicotinamide |
| 122 | 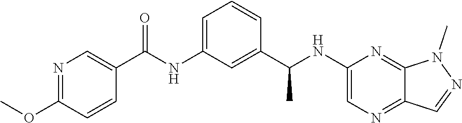 [SMILES: COc1ccc(C(=O)Nc2cccc([C@H](C)Nc3cnc4cnn(C)c4n3)c2)cn1] | (S)-6-methoxy-N-(3-(1-((1- methyl-1H-pyrazolo[3,4-b] pyrazin-6-yl)amino)ethyl) phenyl)nicotinamide |
| 123 | 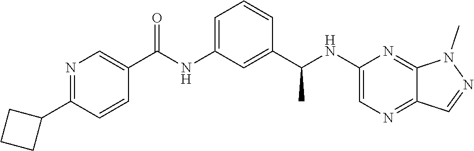 [SMILES: C[C@H](Nc1cnc2cnn(C)c2n1)c1cccc(NC(=O)c2ccc(C3CCC3)nc2)c1] | (S)-6-cyclobutyl-N-(3-(1-((1- methyl-1H-pyrazolo[3,4-b] pyrazin-6-yl)amino)ethyl) phenyl)nicotinamide |
| 124 | 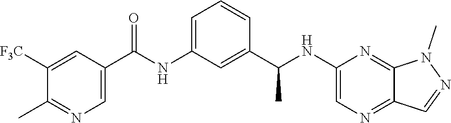 [SMILES: Cc1ncc(C(=O)Nc2cccc([C@H](C)Nc3cnc4cnn(C)c4n3)c2)cc1C(F)(F)F] | (S)-6-methyl-N-(3-(1-((1- methyl-1H-pyrazolo[3,4-b] pyrazin-6-yl)amino)ethyl) phenyl)-5-(trifluoromethyl) nicotinamide |
| 125 | 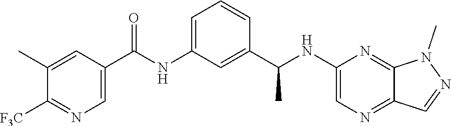 [SMILES: Cc1cc(C(=O)Nc2cccc([C@H](C)Nc3cnc4cnn(C)c4n3)c2)cnc1C(F)(F)F] | (S)-5-methyl-N-(3-(1-((1- methyl-1H-pyrazolo[3,4-b] pyrazin-6-yl)amino)ethyl) phenyl)-6-(trifluoromethyl) nicotinamide |
| 126 | 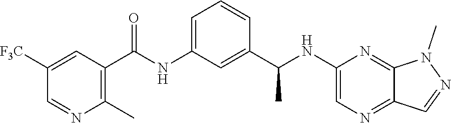 [SMILES: Cc1ncc(C(F)(F)F)cc1C(=O)Nc1cccc([C@H](C)Nc2cnc3cnn(C)c3n2)c1] | (S)-2-methyl-N-(3-(1-((1- methyl-1H-pyrazolo[3,4-b] pyrazin-6-yl)amino)ethyl) phenyl)-5-(trifluoromethyl) nicotinamide |
| 127 | 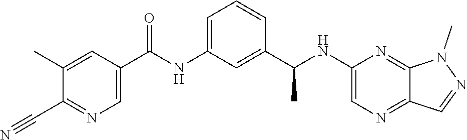 [SMILES: Cc1cc(C(=O)Nc2cccc([C@H](C)Nc3cnc4cnn(C)c4n3)c2)cnc1C#N] | (S)-6-cyano-5-methyl-N-(3-(1- ((1-methyl-1H-pyrazolo[3,4-b] pyrazin-6-yl)amino)ethyl) phenyl)nicotinamide |
| 128 | 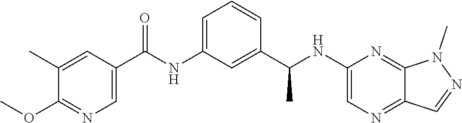 [SMILES: COc1ncc(C(=O)Nc2cccc([C@H](C)Nc3cnc4cnn(C)c4n3)c2)cc1C] | (S)-6-methoxy-5-methyl-N-(3- (1-((1-methyl-1H-pyrazolo[3,4- b]pyrazin-6-yl)amino)ethyl) phenyl)nicotinamide |
| 129 | 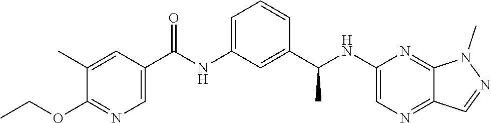 [SMILES: CCOc1ncc(C(=O)Nc2cccc([C@H](C)Nc3cnc4cnn(C)c4n3)c2)cc1C] | (S)-6-ethoxy-5-methyl-N-(3-(1- ((1-methyl-1H-pyrazolo[3,4-b] pyrazin-6-yl)amino)ethyl) phenyl)nicotinamide |
| 130 | 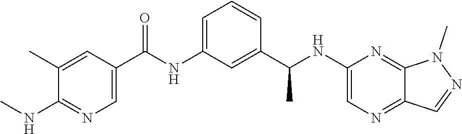 [SMILES: CNc1ncc(C(=O)Nc2cccc([C@H](C)Nc3cnc4cnn(C)c4n3)c2)cc1C] | (S)-5-methyl-N-(3-(1-((1- methyl-1H-pyrazolo[3,4-b] pyrazin-6-yl)amino)ethyl) phenyl)-6-(methylamino) nicotinamide |
| 131 | 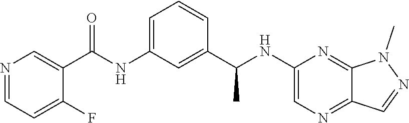 [SMILES: C[C@H](Nc1cnc2cnn(C)c2n1)c1cccc(NC(=O)c2cnccc2F)c1] | (S)-4-fluoro-N-(3-(1-((1-methyl- 1H-pyrazolo[3,4-b]pyrazin-6-yl) amino)ethyl)phenyl) nicotinamide |
| 132 | 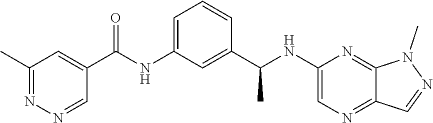 [SMILES: Cc1cc(C(=O)Nc2cccc([C@H](C)Nc3cnc4cnn(C)c4n3)c2)cnn1] | (S)-6-methyl-N-(3-(1-((1- methyl-1H-pyrazolo[3,4-b] pyrazin-6-yl)amino)ethyl) phenyl)pyridazine-4- carboxamide |
| 133 | 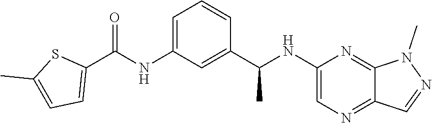 [SMILES: Cc1ccc(C(=O)Nc2cccc([C@H](C)Nc3cnc4cnn(C)c4n3)c2)s1] | (S)-5-methyl-N-(3-(1-((1- methyl-1H-pyrazolo[3,4-b] pyrazin-6-yl)amino)ethyl) phenyl)thiophene-2- carboxamide |
| 134 | 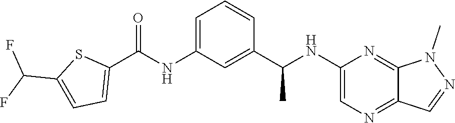 [SMILES: C[C@H](Nc1cnc2cnn(C)c2n1)c1cccc(NC(=O)c2ccc(C(F)F)s2)c1] | (S)-5-(difluoromethyl)-N-(3-(1- ((1-methyl-1H-pyrazolo[3,4-b] pyrazin-6-yl)amino)ethyl) phenyl)thiophene-2- carboxamide |
| 135 | 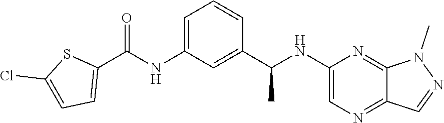 [SMILES: C[C@H](Nc1cnc2cnn(C)c2n1)c1cccc(NC(=O)c2ccc(Cl)s2)c1] | (S)-5-chloro-N-(3-(1-((1- methyl-1H-pyrazolo[3,4-b] pyrazin-6-yl)amino)ethyl) phenyl)thiophene-2- carboxamide |
| 136 | 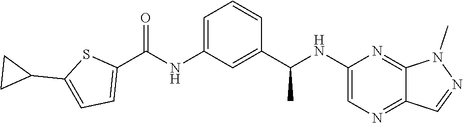 [SMILES: C[C@H](Nc1cnc2cnn(C)c2n1)c1cccc(NC(=O)c2ccc(C3CC3)s2)c1] | (S)-5-cyclopropyl-N-(3-(1-((1- methyl-1H-pyrazolo[3,4-b] pyrazin-6-yl)amino)ethyl) phenyl)thiophene-2- carboxamide |
| 137 | 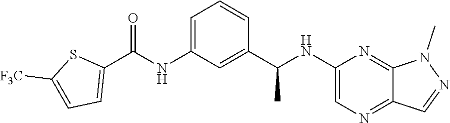 [SMILES: C[C@H](Nc1cnc2cnn(C)c2n1)c1cccc(NC(=O)c2ccc(C(F)(F)F)s2)c1] | (S)-N-(3-(1-((1-methyl-1H- pyrazolo[3,4-b]pyrazin-6-yl) amino)ethyl)phenyl)-5- (trifluoromethyl)thiophene-2- carboxamide |
| 138 | 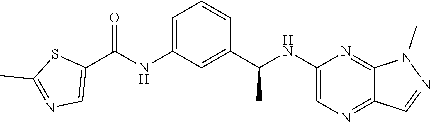 [SMILES: Cc1ncc(C(=O)Nc2cccc([C@H](C)Nc3cnc4cnn(C)c4n3)c2)s1] | (S)-2-methyl-N-(3-(1-((1- methyl-1H-pyrazolo[3,4-b] pyrazin-6-yl)amino)ethyl) phenyl)thiazole-5-carboxamide |
| 139 | 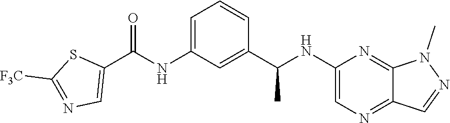 [SMILES: C[C@H](Nc1cnc2cnn(C)c2n1)c1cccc(NC(=O)c2cnc(C(F)(F)F)s2)c1] | (S)-N-(3-(1-((1-methyl-1H- pyrazolo[3,4-b]pyrazin-6-yl) amino)ethyl)phenyl)-2- (trifluoromethyl)thiazole-5- carboxamide |
| 140 | 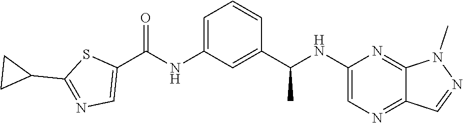 [SMILES: C[C@H](Nc1cnc2cnn(C)c2n1)c1cccc(NC(=O)c2cnc(C3CC3)s2)c1] | (S)-2-cyclopropyl-N-(3-(1-((1- methyl-1H-pyrazolo[3,4-b] pyrazin-6-yl)amino)ethyl) phenyl)thiazole-5-carboxamide |
| 141 | 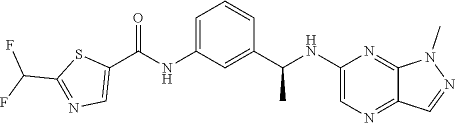 [SMILES: C[C@H](Nc1cnc2cnn(C)c2n1)c1cccc(NC(=O)c2cnc(C(F)F)s2)c1] | (S)-2-(difluoromethyl)-N-(3-(1- ((1-methyl-1H-pyrazolo[3,4-b] pyrazin-6-yl)amino)ethyl) phenyl)thiazole-5-carboxamide |
| 142 | 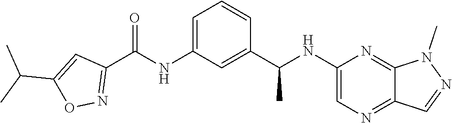 [SMILES: CC(C)c1cc(C(=O)Nc2cccc([C@H](C)Nc3cnc4cnn(C)c4n3)c2)no1] | (S)-5-isopropyl-N-(3-(1-((1- methyl-1H-pyrazolo[3,4-b] pyrazin-6-yl)amino)ethyl) phenyl)isoxazole-3- carboxamide |
| 143 | 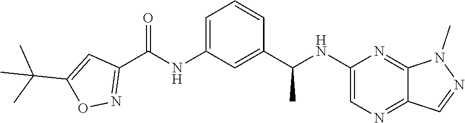 [SMILES: C[C@H](Nc1cnc2cnn(C)c2n1)c1cccc(NC(=O)c2cc(C(C)(C)C)on2)c1] | (S)-5-(tert-butyl)-N-(3-(1-((1- methyl-1H-pyrazolo[3,4-b] pyrazin-6-yl)amino)ethyl) phenyl)isoxazole-3- carboxamide |
| 144 | 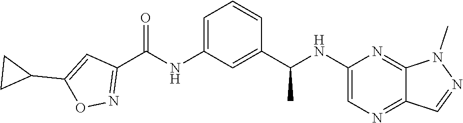 [SMILES: C[C@H](Nc1cnc2cnn(C)c2n1)c1cccc(NC(=O)c2cc(C3CC3)on2)c1] | (S)-5-cyclopropyl-N-(3-(1-((1- methyl-1H-pyrazolo[3,4-b] pyrazin-6-yl)amino)ethyl) phenyl)isoxazole-3- carboxamide |
| 145 | 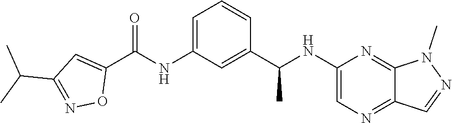 [SMILES: CC(C)c1cc(C(=O)Nc2cccc([C@H](C)Nc3cnc4cnn(C)c4n3)c2)on1] | (S)-3-isopropyl-N-(3-(1-((1- methyl-1H-pyrazolo[3,4-b] pyrazin-6-yl)amino)ethyl) phenyl)isoxazole-5- carboxamide |
| 146 | 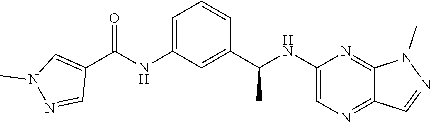 [SMILES: C[C@H](Nc1cnc2cnn(C)c2n1)c1cccc(NC(=O)c2cnn(C)c2)c1] | (S)-1-methyl-N-(3-(1-((1- methyl-1H-pyrazolo[3,4-b] pyrazin-6-yl)amino)ethyl) phenyl)-1H-pyrazole-4- carboxamide |
| 147 | 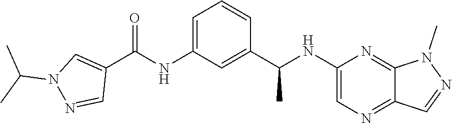 [SMILES: CC(C)n1cc(C(=O)Nc2cccc([C@H](C)Nc3cnc4cnn(C)c4n3)c2)cn1] | (S)-1-isopropyl-N-(3-(1-((1- methyl-1H-pyrazolo[3,4-b] pyrazin-6-yl)amino)ethyl) phenyl)-1H-pyrazole-4- carboxamide |
| 148 | 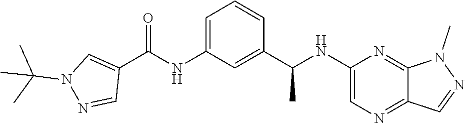 [SMILES: C[C@H](Nc1cnc2cnn(C)c2n1)c1cccc(NC(=O)c2cnn(C(C)(C)C)c2)c1] | (S)-1-(tert-butyl)-N-(3-(1-((1- methyl-1H-pyrazolo[3,4-b] pyrazin-6-yl)amino)ethyl) phenyl)-1H-pyrazole-4- carboxamide |
| 149 | 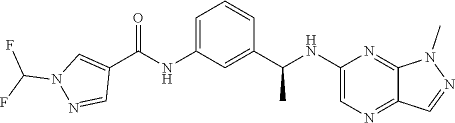 [SMILES: C[C@H](Nc1cnc2cnn(C)c2n1)c1cccc(NC(=O)c2cnn(C(F)F)c2)c1] | (S)-1-(difluoromethyl)-N-(3-(1- ((1-methyl-1H-pyrazolo[3,4-b] pyrazin-6-yl)amino)ethyl) phenyl)-1H-pyrazole-4- carboxamide |
| 150 | 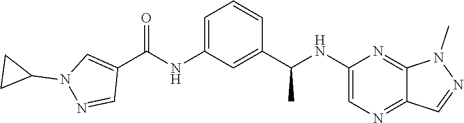 [SMILES: C[C@H](Nc1cnc2cnn(C)c2n1)c1cccc(NC(=O)c2cnn(C3CC3)c2)c1] | (S)-1-cyclopropyl-N-(3-(1-((1- methyl-1H-pyrazolo[3,4-b] pyrazin-6-yl)amino)ethyl) phenyl)-1H-pyrazole-4- carboxamide |
| 151 | 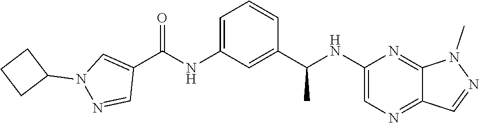 [SMILES: C[C@H](Nc1cnc2cnn(C)c2n1)c1cccc(NC(=O)c2cnn(C3CCC3)c2)c1] | (S)-1-cyclobutyl-N-(3-(1-((1- methyl-1H-pyrazolo[3,4-b] pyrazin-6-yl)amino)ethyl) phenyl)-1H-pyrazole-4- carboxamide |
| 152 | 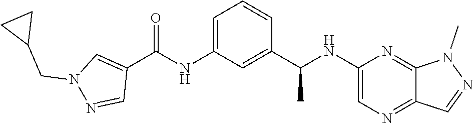 [SMILES: C[C@H](Nc1cnc2cnn(C)c2n1)c1cccc(NC(=O)c2cnn(CC3CC3)c2)c1] | (S)-1-(cyclopropylmethyl)-N-(3- (1-((1-methyl-1H-pyrazolo[3,4- b]pyrazin-6-yl)amino)ethyl) phenyl)-1H-pyrazole-4- carboxamide |
| 153 | 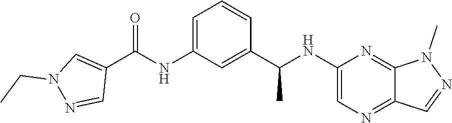 [SMILES: CCn1cc(C(=O)Nc2cccc([C@H](C)Nc3cnc4cnn(C)c4n3)c2)cn1] | (S)-1-ethyl-N-(3-(1-((1-methyl- 1H-pyrazolo[3,4-b]pyrazin-6-yl) amino)ethyl)phenyl)-1H- pyrazole-4-carboxamide |
| 154 | 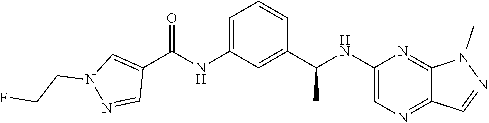 [SMILES: C[C@H](Nc1cnc2cnn(C)c2n1)c1cccc(NC(=O)c2cnn(CCF)c2)c1] | (S)-1-(2-fluoroethyl)-N-(3-(1- ((1-methyl-1H-pyrazolo[3,4-b] pyrazin-6-yl)amino)ethyl) phenyl)-1H-pyrazole-4- carboxamide |
| 155 | 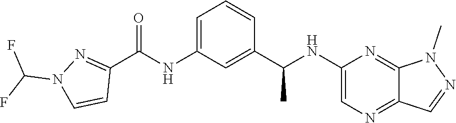 [SMILES: C[C@H](Nc1cnc2cnn(C)c2n1)c1cccc(NC(=O)c2ccn(C(F)F)n2)c1] | (S)-1-(difluoromethyl)-N-(3-(1- ((1-methyl-1H-pyrazolo[3,4-b] pyrazin-6-yl)amino)ethyl) phenyl)-1H-pyrazole-3- carboxamide |
| 156 | 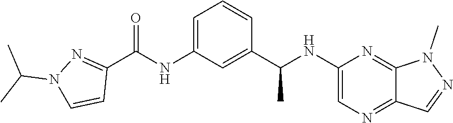 [SMILES: CC(C)n1ccc(C(=O)Nc2cccc([C@H](C)Nc3cnc4cnn(C)c4n3)c2)n1] | (S)-1-isopropyl-N-(3-(1-((1- methyl-1H-pyrazolo[3,4-b] pyrazin-6-yl)amino)ethyl) phenyl)-1H-pyrazole-3- carboxamide |
| 157 | 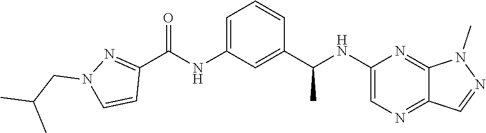 [SMILES: CC(C)Cn1ccc(C(=O)Nc2cccc([C@H](C)Nc3cnc4cnn(C)c4n3)c2)n1] | (S)-1-isobutyl-N-(3-(1-((1- methyl-1H-pyrazolo[3,4-b] pyrazin-6-yl)amino)ethyl) phenyl)-1H-pyrazole-3- carboxamide |
| 158 | 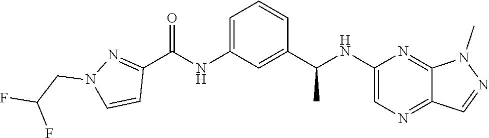 [SMILES: C[C@H](Nc1cnc2cnn(C)c2n1)c1cccc(NC(=O)c2ccn(CC(F)F)n2)c1] | (S)-1-(2,2-difluoroethyl)-N-(3- (1-((1-methyl-1H-pyrazolo[3,4- b]pyrazin-6-yl)amino)ethyl) phenyl)-1H-pyrazole-3- carboxamide |
| 159 | 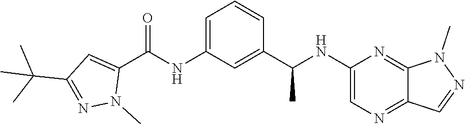 [SMILES: C[C@H](Nc1cnc2cnn(C)c2n1)c1cccc(NC(=O)c2cc(C(C)(C)C)nn2C)c1] | (S)-3-(tert-butyl)-1-methyl-N- (3-(1-((1-methyl-1H- pyrazolo[3,4-b]pyrazin-6-yl) amino)ethyl)phenyl)-1H- pyrazole-5-carboxamide |
| 160 | 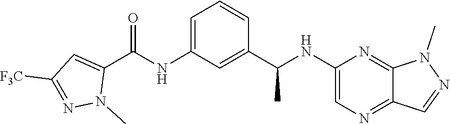 [SMILES: C[C@H](Nc1cnc2cnn(C)c2n1)c1cccc(NC(=O)c2cc(C(F)(F)F)nn2C)c1] | (S)-1-methyl-N-(3-(1-((1- methyl-1H-pyrazolo[3,4-b] pyrazin-6-yl)amino)ethyl) phenyl)-3-(trifluoromethyl)-1H- pyrazole-5-carboxamide |
| 161 | 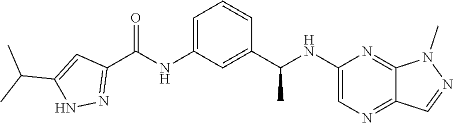 [SMILES: CC(C)c1cc(C(=O)Nc2cccc([C@H](C)Nc3cnc4cnn(C)c4n3)c2)n[nH]1] | (S)-5-isopropyl-N-(3-(1-((1- methyl-1H-pyrazolo[3,4-b] pyrazin-6-yl)amino)ethyl) phenyl)-1H-pyrazole-3- carboxamide |
| 162 | 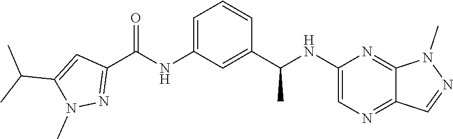 [SMILES: CC(C)c1cc(C(=O)Nc2cccc([C@H](C)Nc3cnc4cnn(C)c4n3)c2)nn1C] | (S)-5-isopropyl-1-methyl-N-(3- (1-((1-methyl-1H-pyrazolo[3,4- b]pyrazin-6-yl)amino)ethyl) phenyl)-1H-pyrazole-3- carboxamide |
| 163 | 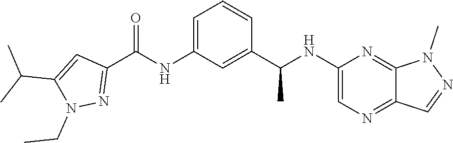 [SMILES: CCn1nc(C(=O)Nc2cccc([C@H](C)Nc3cnc4cnn(C)c4n3)c2)cc1C(C)C] | (S)-1-ethyl-5-isopropyl-N-(3-(1- ((1-methyl-1H-pyrazolo[3,4-b] pyrazin-6-yl)amino)ethyl) phenyl)-1H-pyrazole-3- carboxamide |
| 164 | 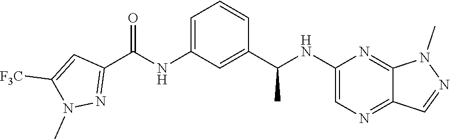 [SMILES: C[C@H](Nc1cnc2cnn(C)c2n1)c1cccc(NC(=O)c2cc(C(F)(F)F)n(C)n2)c1] | (S)-1-methyl-N-(3-(1-((1- methyl-1H-pyrazolo[3,4-b] pyrazin-6-yl)amino)ethyl) phenyl)-5-(trifluoromethyl)-1H- pyrazole-3-carboxamide |
| 165 | 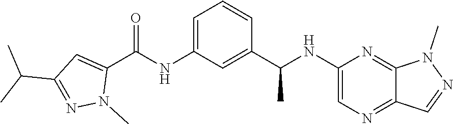 [SMILES: CC(C)c1cc(C(=O)Nc2cccc([C@H](C)Nc3cnc4cnn(C)c4n3)c2)n(C)n1] | (S)-3-isopropyl-1-methyl-N-(3- (1-((1-methyl-1H-pyrazolo[3,4- 6]pyrazin-6-yl)amino)ethyl) phenyl)-1H-pyrazole-5- carboxamide |
| 166 | 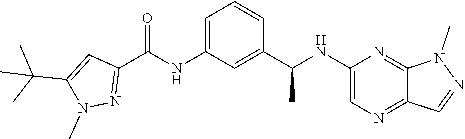 [SMILES: C[C@H](Nc1cnc2cnn(C)c2n1)c1cccc(NC(=O)c2cc(C(C)(C)C)n(C)n2)c1] | (S)-5-(tert-butyl)-1-methyl-N- (3-(1-((1-methyl-1H- pyrazolo[3,4-b]pyrazin-6-yl) amino)ethyl)phenyl)-1H- pyrazole-3-carboxamide |
| 167 | 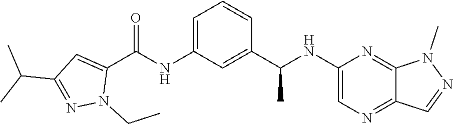 [SMILES: CCn1nc(C(C)C)cc1C(=O)Nc1cccc([C@H](C)Nc2cnc3cnn(C)c3n2)c1] | (S)-1-ethyl-3-isopropyl-N-(3-(1- ((1-methyl-1H-pyrazolo[3,4-b] pyrazin-6-yl)amino)ethyl) phenyl)-1H-pyrazole-5- carboxamide |
| 168 | 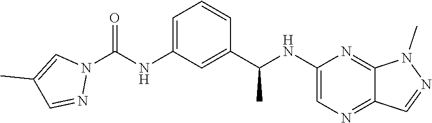 [SMILES: Cc1cnn(C(=O)Nc2cccc([C@H](C)Nc3cnc4cnn(C)c4n3)c2)c1] | (S)-4-methyl-N-(3-(1-((1- methyl-1H-pyrazolo[3,4-b] pyrazin-6-yl)amino)ethyl) phenyl)-1H-pyrazole-1- carboxamide |
| 169 | 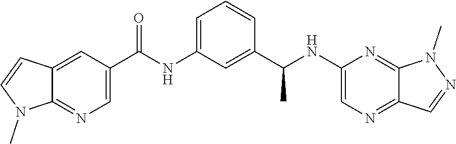 [SMILES: C[C@H](Nc1cnc2cnn(C)c2n1)c1cccc(NC(=O)c2cnc3c(ccn3C)c2)c1] | (S)-1-methyl-N-(3-(1-((1- methyl-1H-pyrazolo[3,4-b] pyrazin-6-yl)amino)ethyl) phenyl)-1H-pyrrolo[2,3-b] pyridine-5-carboxamide |
| 170 | 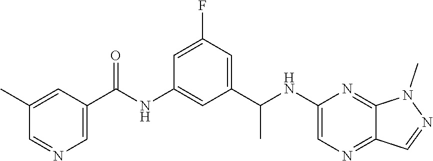 [SMILES: Cc1cncc(C(=O)Nc2cc(F)cc(C(C)Nc3cnc4cnn(C)c4n3)c2)c1] | N-(3-fluoro-5-(1-((1-methyl-1H- pyrazolo[3,4-b]pyrazin-6-yl) amino)ethyl)phenyl)-5- methylnicotinamide |
| 171 | 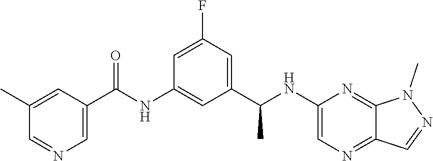 [SMILES: Cc1cncc(C(=O)Nc2cc(F)cc([C@H](C)Nc3cnc4cnn(C)c4n3)c2)c1] | (S)-N-(3-fluoro-5-(1-((1-methyl- 1H-pyrazolo[3,4-b]pyrazin-6-yl) amino)ethyl)phenyl)-5- methylnicotinamide |
| 172 | 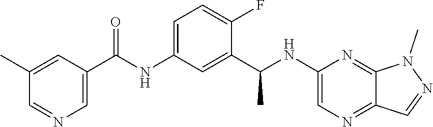 [SMILES: Cc1cncc(C(=O)Nc2ccc(F)c([C@H](C)Nc3cnc4cnn(C)c4n3)c2)c1] | (S)-N-(3-fluoro-3-(1-((1-methyl- 1H-pyrazolo[3,4-b]pyrazin-6-yl) amino)ethyl)phenyl)-5- methylnicotinamide |
| 173 | 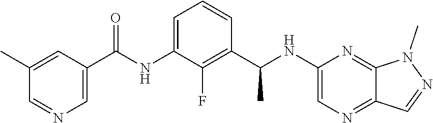 [SMILES: Cc1cncc(C(=O)Nc2cccc([C@H](C)Nc3cnc4cnn(C)c4n3)c2F)c1] | (S)-N-(2-fluoro-3-(1-((1-methyl- 1H-pyrazolo[3,4-b]pyrazin-6-yl) amino)ethyl)phenyl)-5- methylnicotinamide |
| 174 | 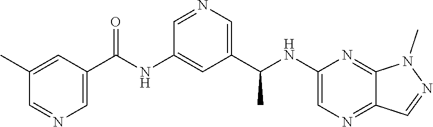 [SMILES: Cc1cncc(C(=O)Nc2cncc([C@H](C)Nc3cnc4cnn(C)c4n3)c2)c1] | (S)-5-methyl-N-(5-(1-((1- methyl-1H-pyrazolo[3,4-b] pyrazin-6-yl)amino)ethyl) pyridin-3-yl)nicotinamide |
| 175 | 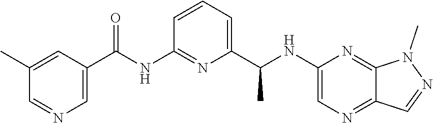 [SMILES: Cc1cncc(C(=O)Nc2cccc([C@H](C)Nc3cnc4cnn(C)c4n3)n2)c1] | (S)-5-methyl-N-(6-(1-((1- methyl-1H-pyrazolo[3,4-b] pyrazin-6-yl)amino)ethyl) pyridin-2-yl)nicotinamide |
| 176 | 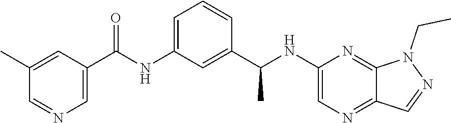 [SMILES: CCn1ncc2ncc(N[C@@H](C)c3cccc(NC(=O)c4cncc(C)c4)c3)nc21] | (S)-N-(3-(1-((1-ethyl-1H- pyrazolo[3,4-b]pyrazin-6-yl) amino)ethyl)phenyl)-5- methylnicotinamide |
| 177 | 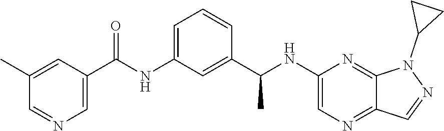 [SMILES: Cc1cncc(C(=O)Nc2cccc([C@H](C)Nc3cnc4cnn(C5CC5)c4n3)c2)c1] | (S)-N-(3-(1-((1-cyclopropyl-1H- pyrazolo[3,4-b]pyrazin-6-yl) amino)ethyl)phenyl)-5- methylnicotinamide |
| 178 | 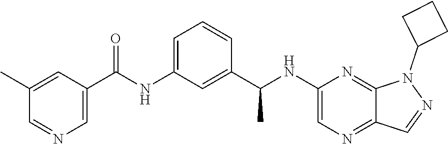 [SMILES: Cc1cncc(C(=O)Nc2cccc([C@H](C)Nc3cnc4cnn(C5CCC5)c4n3)c2)c1] | (S)-N-(3-(1-((1-cyclobutyl-1H- pyrazolo[3,4-b]pyrazin-6-yl) amino)ethyl)phenyl)-5- methylnicotinamide |
| 179 | 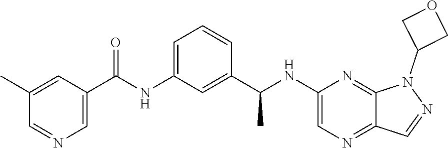 [SMILES: Cc1cncc(C(=O)Nc2cccc([C@H](C)Nc3cnc4cnn(C5COC5)c4n3)c2)c1] | (S)-5-methyl-N-(3-(1-((1- (oxetan-3-yl)-1H-pyrazolo[3,4- b]pyrazin-6-yl)amino)ethyl) phenyl)nicotinamide |
| 180 | 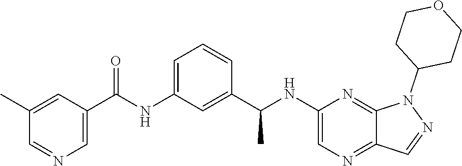 [SMILES: Cc1cncc(C(=O)Nc2cccc([C@H](C)Nc3cnc4cnn(C5CCOCC5)c4n3)c2)c1] | (S)-5-methyl-N-(3-(1-((1- (tetrahydro-2H-pyran-4-yl)-1H- pyrazolo[3,4-b]pyrazin-6-yl) amino)ethyl)phenyl) nicotinamide |
| 181 | 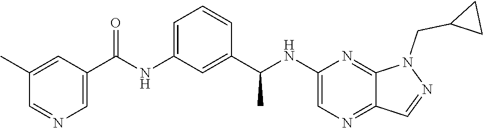 [SMILES: Cc1cncc(C(=O)Nc2cccc([C@H](C)Nc3cnc4cnn(CC5CC5)c4n3)c2)c1] | (S)-N-(3-(1-((1- (cyclopropylmethyl)-1H- pyrazolo[3,4-b]pyrazin-6-yl) amino)ethyl)phenyl)-5- methylnicotinamide |
| 182 | 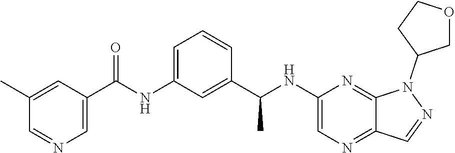 [SMILES: Cc1cncc(C(=O)Nc2cccc([C@H](C)Nc3cnc4cnn(C5CCOC5)c4n3)c2)c1] | 5-methyl-N-(3-((1S)-1-((1- (tetrahydrofuran-3-yl)-1H- pyrazolo[3,4-b]pyrazin-6-yl) amino)ethyl)phenyl) nicotinamide |
| 183 | 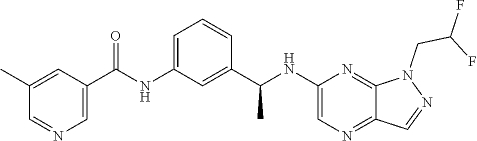 [SMILES: Cc1cncc(C(=O)Nc2cccc([C@H](C)Nc3cnc4cnn(CC(F)F)c4n3)c2)c1] | (S)-N-(3-(1-((1-(2,2- difluoroethyl)-1H-pyrazolo[3,4- b]pyrazin-6-yl)amino)ethyl) phenyl)-5-methylnicotinamide |
| 184 | 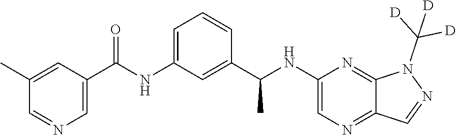 [SMILES: [2H]C([2H])([2H])n1ncc2ncc(N[C@@H](C)c3cccc(NC(=O)c4cncc(C)c4)c3)nc21] | (S)-5-methyl-N-(3-(1-((1- (methyl-d3)-1H-pyrazolo[3,4-b] pyrazin-6-yl)amino)ethyl) phenyl)nicotinamide |
| 185 | 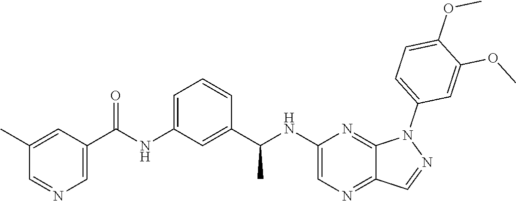 | (S)-N-(3-(1-((1-(3,4- dimethoxyphenyl)-1H- pyrazolo[3,4-b]pyrazin-6-yl) amino)ethyl)phenyl)-5- methylnicotinamide |
| 186 | 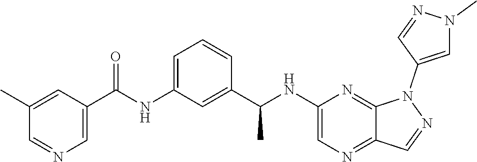 | (S)-5-methyl-N-(3-(1-((1-(1- methyl-1H-pyrazol-4-yl)-1H- pyrazolo[3,4-b]pyrazin-6-yl) amino)ethyl)phenyl) nicotinamide |
| 187 | 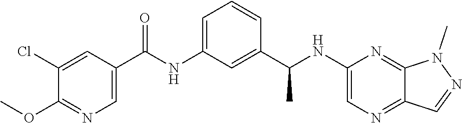 [SMILES: COc1ncc(C(=O)Nc2cccc([C@H](C)Nc3cnc4cnn(C)c4n3)c2)cc1Cl] | (S)-5-chloro-6-methoxy-N-(3- (1-((1-methyl-1H-pyrazolo[3,4- b]pyrazin-6-yl)amino)ethyl) phenyl)nicotinamide |
| 188 | 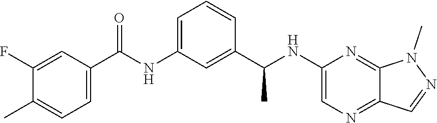 [SMILES: Cc1ccc(C(=O)Nc2cccc([C@H](C)Nc3cnc4cnn(C)c4n3)c2)cc1F] | (S)-3-fluoro-4-methyl-N-(3-(1- ((1-methyl-1H-pyrazolo[3,4-b] pyrazin-6-yl)amino)ethyl) phenyl)benzamide |
| 189 | 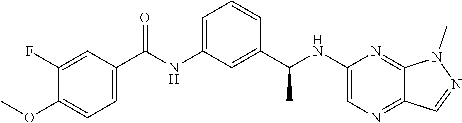 [SMILES: COc1ccc(C(=O)Nc2cccc([C@H](C)Nc3cnc4cnn(C)c4n3)c2)cc1F] | (S)-3-fluoro-4-methoxy-N-(3-(1- ((1-methyl-1H-pyrazolo[3,4-b] pyrazin-6-yl)amino)ethyl) phenyl)benzamide |
| 190 | 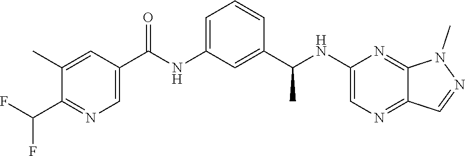 [SMILES: Cc1cc(C(=O)Nc2cccc([C@H](C)Nc3cnc4cnn(C)c4n3)c2)cnc1C(F)F] | (S)-6-(difluoromethyl)-5- methyl-N-(3-(1-((1-methyl-1H- pyrazolo[3,4-b]pyrazin-6-yl) amino)ethyl)phenyl) nicotinamide |
| 191 | 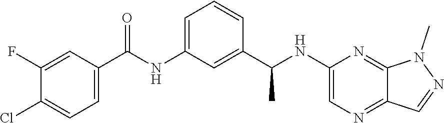 [SMILES: C[C@H](Nc1cnc2cnn(C)c2n1)c1cccc(NC(=O)c2ccc(Cl)c(F)c2)c1] | (S)-4-chloro-3-fluoro-N-(3-(1- ((1-methyl-1H-pyrazolo[3,4-b] pyrazin-6-yl)amino)ethyl) phenyl)benzamide |
| 192 | 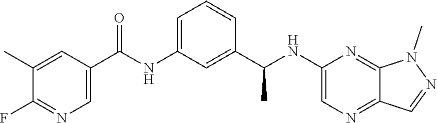 [SMILES: Cc1cc(C(=O)Nc2cccc([C@H](C)Nc3cnc4cnn(C)c4n3)c2)cnc1F] | (S)-6-fluoro-5-methyl-N-(3-(1- ((1-methyl-1H-pyrazolo[3,4-b] pyrazin-6-yl)amino)ethyl) phenyl)nicotinamide |
| 193 | 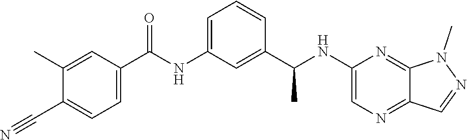 [SMILES: Cc1cc(C(=O)Nc2cccc([C@H](C)Nc3cnc4cnn(C)c4n3)c2)ccc1C#N] | (S)-4-cyano-3-methyl-N-(3-(1- ((1-methyl-1H-pyrazolo[3,4-b] pyrazin-6-yl)amino)ethyl) phenyl)benzamide |
| 194 | 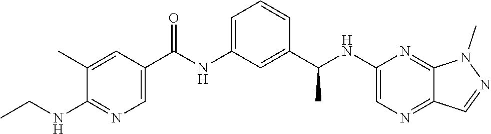 [SMILES: CCNc1ncc(C(=O)Nc2cccc([C@H](C)Nc3cnc4cnn(C)c4n3)c2)cc1C] | (S)-6-(ethylamino)-5-methyl-N- (3-(1-((1-methyl-1H- pyrazolo[3,4-b]pyrazin-6-yl) amino)ethyl)phenyl) nicotinamide |
| 195 | 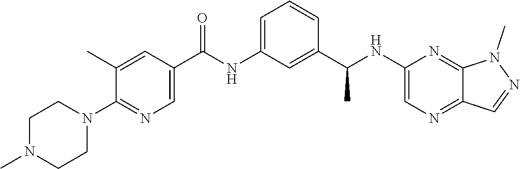 [SMILES: Cc1cc(C(=O)Nc2cccc([C@H](C)Nc3cnc4cnn(C)c4n3)c2)cnc1N1CCN(C)CC1] | (S)-5-methyl-N-(3-(1-((1- methyl-1H-pyrazolo[3,4-b] pyrazin-6-yl)amino)ethyl) phenyl)-6-(4-methylpiperazin-1- yl)nicotinamide |
| 196 | 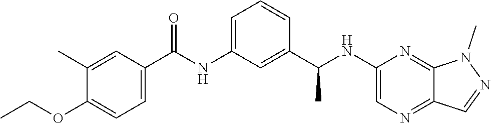 [SMILES: CCOc1ccc(C(=O)Nc2cccc([C@H](C)Nc3cnc4cnn(C)c4n3)c2)cc1C] | (S)-4-ethoxy-3-methyl-N-(3-(1- ((1-methyl-1H-pyrazolo[3,4-b] pyrazin-6-yl)amino)ethyl) phenyl)benzamide |
| 197 | 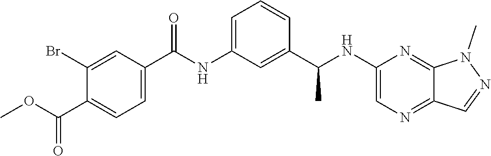 [SMILES: COC(=O)c1ccc(C(=O)Nc2cccc([C@H](C)Nc3cnc4cnn(C)c4n3)c2)cc1Br] | methyl (S)-2-bromo-4-((3-(1- ((1-methyl-1H-pyrazolo[3,4-b] pyrazin-6-yl)amino)ethyl) phenyl)carbamoyl)benzoate |
| 198 | 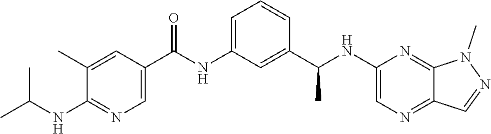 [SMILES: Cc1cc(C(=O)Nc2cccc([C@H](C)Nc3cnc4cnn(C)c4n3)c2)cnc1NC(C)C] | (S)-6-(isopropylamino)-5- methyl-N-(3-(1-((1-methyl-1H- pyrazolo[3,4-b]pyrazin-6-yl) amino)ethyl)phenyl) nicotinamide |
| 199 | 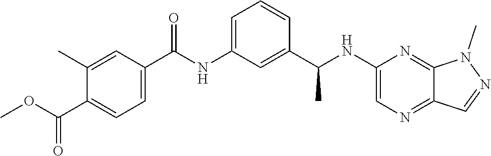 [SMILES: COC(=O)c1ccc(C(=O)Nc2cccc([C@H](C)Nc3cnc4cnn(C)c4n3)c2)cc1C] | methyl (S)-2-methyl-4-((3-(1- ((1-methyl-1H-pyrazolo[3,4-b] pyrazin-6-yl)amino)ethyl) phenyl)carbamoyl)benzoate |
| 200 | 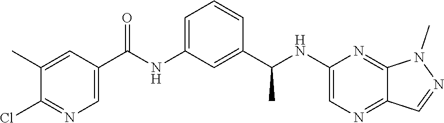 [SMILES: Cc1cc(C(=O)Nc2cccc([C@H](C)Nc3cnc4cnn(C)c4n3)c2)cnc1Cl] | (S)-6-chloro-5-methyl-N-(3-(1- ((1-methyl-1H-pyrazolo[3,4-b] pyrazin-6-yl)amino)ethyl) phenyl)nicotinamide |
| 201 | 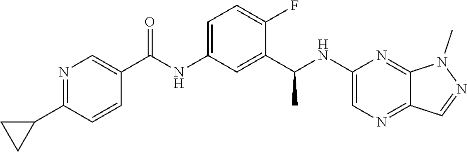 [SMILES: C[C@H](Nc1cnc2cnn(C)c2n1)c1cc(NC(=O)c2ccc(C3CC3)nc2)ccc1F] | (S)-6-cyclopropyl-N-(4-fluoro- 3-(1-((1-methyl-1H- pyrazolo[3,4-b]pyrazin-6-yl) amino)ethyl)phenyl) nicotinamide |
| 202 | 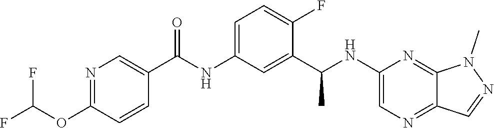 [SMILES: C[C@H](Nc1cnc2cnn(C)c2n1)c1cc(NC(=O)c2ccc(OC(F)F)nc2)ccc1F] | (S)-6-(difluoromethoxy)-N-(4- fluoro-3-(1-((1-methyl-1H- pyrazolo[3,4-b]pyrazin-6-yl) amino)ethyl)phenyl) nicotinamide |
| 203 | 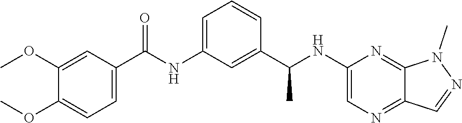 [SMILES: COc1ccc(C(=O)Nc2cccc([C@H](C)Nc3cnc4cnn(C)c4n3)c2)cc1OC] | (S)-3,4-dimethoxy-N-(3-(1-((1- methyl-1H-pyrazolo[3,4-b] pyrazin-6-yl)amino)ethyl) phenyl)benzamide |
| 204 | 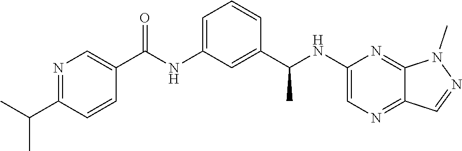 [SMILES: CC(C)c1ccc(C(=O)Nc2cccc([C@H](C)Nc3cnc4cnn(C)c4n3)c2)cn1] | (S)-6-isopropyl-N-(3-(1-((1- methyl-1H-pyrazolo[3,4-b] pyrazin-6-yl)amino)ethyl) phenyl)nicotinamide |
| 205 | 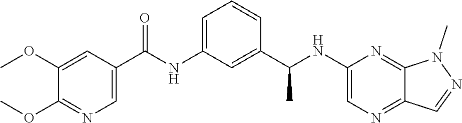 [SMILES: COc1cc(C(=O)Nc2cccc([C@H](C)Nc3cnc4cnn(C)c4n3)c2)cnc1OC] | (S)-5,6-dimethoxy-N-(3-(1-((1- methyl-1H-pyrazolo[3,4-b] pyrazin-6-yl)amino)ethyl) phenyl)nicotinamide |
| 206 | 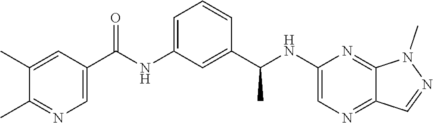 [SMILES: Cc1cc(C(=O)Nc2cccc([C@H](C)Nc3cnc4cnn(C)c4n3)c2)cnc1C] | (S)-5,6-dimethyl-N-(3-(1-((1- methyl-1H-pyrazolo[3,4-b] pyrazin-6-yl)amino)ethyl) phenyl)nicotinamide |
| 207 | 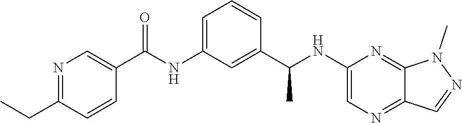 [SMILES: CCc1ccc(C(=O)Nc2cccc([C@H](C)Nc3cnc4cnn(C)c4n3)c2)cn1] | (S)-6-ethyl-N-(3-(1-((1-methyl- 1H-pyrazolo[3,4-b]pyrazin-6-yl) amino)ethyl)phenyl) nicotinamide |
| 208 | 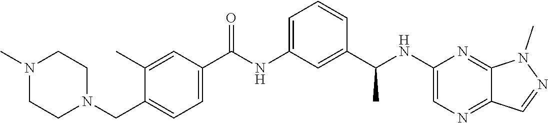 [SMILES: Cc1cc(C(=O)Nc2cccc([C@H](C)Nc3cnc4cnn(C)c4n3)c2)ccc1CN1CCN(C)CC1] | (S)-3-methyl-N-(3-(1-((1- methyl-1H-pyrazolo[3,4-b] pyrazin-6-yl)amino)ethyl) phenyl)-4-((4-methylpiperazin- 1-yl)methyl)benzamide |
| 209 | 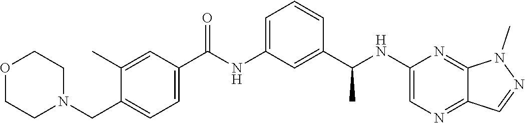 [SMILES: Cc1cc(C(=O)Nc2cccc([C@H](C)Nc3cnc4cnn(C)c4n3)c2)ccc1CN1CCOCC1] | (S)-3-methyl-N-(3-(1-((1- methyl-1H-pyrazolo[3,4-b] pyrazin-6-yl)amino)ethyl) phenyl)-4-(morpholinomethyl) benzamide |
| 210 | 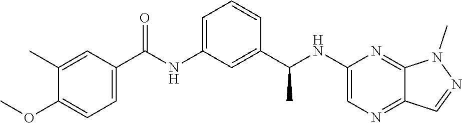 [SMILES: COc1ccc(C(=O)Nc2cccc([C@H](C)Nc3cnc4cnn(C)c4n3)c2)cc1C] | (S)-4-methoxy-3-methyl-N-(3- (1-((1-methyl-1H-pyrazolo[3,4- b]pyrazin-6-yl)amino)ethyl) phenyl)benzamide |
| 211 | 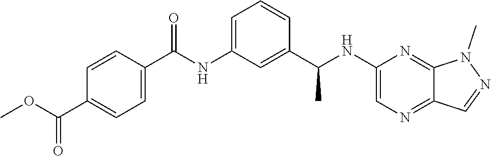 [SMILES: COC(=O)c1ccc(C(=O)Nc2cccc([C@H](C)Nc3cnc4cnn(C)c4n3)c2)cc1] | methyl (S)-4-((3-(1-((1-methyl- 1H-pyrazolo[3,4-b]pyrazin-6-yl) amino)ethyl)phenyl)carbamoyl) benzoate |
| 212 | 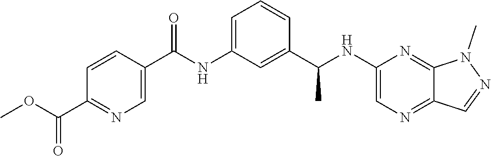 [SMILES: COC(=O)c1ccc(C(=O)Nc2cccc([C@H](C)Nc3cnc4cnn(C)c4n3)c2)cn1] | methyl (S)-5-((3-(1-((1-methyl- 1H-pyrazolo[3,4-b]pyrazin-6-yl) amino)ethyl)phenyl)carbamoyl) picolinate |
| 213 | 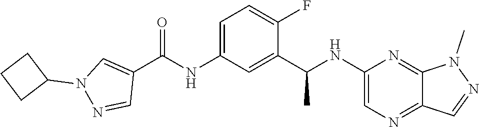 [SMILES: C[C@H](Nc1cnc2cnn(C)c2n1)c1cc(NC(=O)c2cnn(C3CCC3)c2)ccc1F] | (S)-1-cyclobutyl-N-(4-fluoro-3- (1-((1-methyl-1H-pyrazolo[3,4- b]pyrazin-6-yl)amino)ethyl) phenyl)-1H-pyrazole-4- carboxamide |
| 214 | 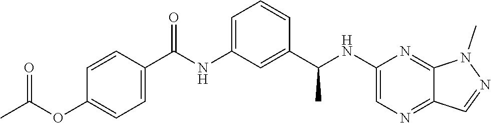 [SMILES: CC(=O)Oc1ccc(C(=O)Nc2cccc([C@H](C)Nc3cnc4cnn(C)c4n3)c2)cc1] | (S)-4-((3-(1-((1-methyl-1H- pyrazolo[3,4-b]pyrazin-6-yl) amino)ethyl)phenyl)carbamoyl) phenyl acetate |
| 215 | 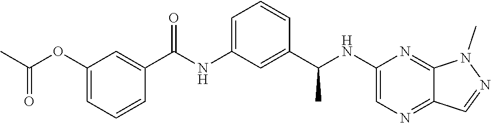 [SMILES: CC(=O)Oc1cccc(C(=O)Nc2cccc([C@H](C)Nc3cnc4cnn(C)c4n3)c2)c1] | (S)-3-((3-(1-((1-methyl-1H- pyrazolo[3,4-b]pyrazin-6-yl) amino)ethyl)phenyl)carbamoyl) phenyl acetate |
| 216 | 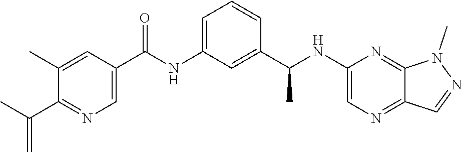 [SMILES: C=C(C)c1ncc(C(=O)Nc2cccc([C@H](C)Nc3cnc4cnn(C)c4n3)c2)cc1C] | (S)-5-methyl-N-(3-(1-((1- methyl-1H-pyrazolo[3,4-b] pyrazin-6-yl)amino)ethyl) phenyl)-6-(prop-1-en-2-yl) nicotinamide |
| 217 | 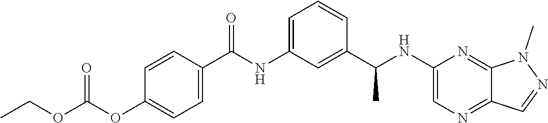 [SMILES: CCOC(=O)Oc1ccc(C(=O)Nc2cccc([C@H](C)Nc3cnc4cnn(C)c4n3)c2)cc1] | (S)-ethyl (4-((3-(1-((1-methyl- 1H-pyrazolo[3,4-b]pyrazin-6-yl) amino)ethyl)phenyl)carbamoyl) phenyl) carbonate |
| 218 | 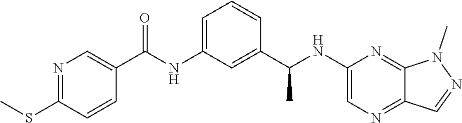 [SMILES: CSc1ccc(C(=O)Nc2cccc([C@H](C)Nc3cnc4cnn(C)c4n3)c2)cn1] | (S)-N-(3-(1-((1-methyl-1H- pyrazolo[3,4-b]pyrazin-6-yl) amino)ethyl)phenyl)-6- (methylthio)nicotinamide |
| 219 | 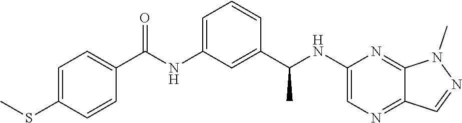 [SMILES: CSc1ccc(C(=O)Nc2cccc([C@H](C)Nc3cnc4cnn(C)c4n3)c2)cc1] | (S)-N-(3-(1-((1-methyl-1H- pyrazolo[3,4-b]pyrazin-6-yl) amino)ethyl)phenyl)-4- (methylthio)benzamide |
| 220 | 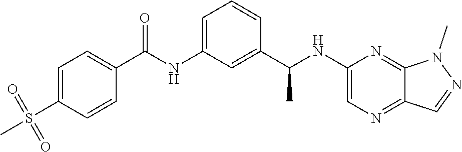 [SMILES: C[C@H](Nc1cnc2cnn(C)c2n1)c1cccc(NC(=O)c2ccc(S(C)(=O)=O)cc2)c1] | (S)-N-(3-(1-((1-methyl-1H- pyrazolo[3,4-b]pyrazin-6-yl) amino)ethyl)phenyl)-4- (methylsulfonyl)benzamide |
| 221 | 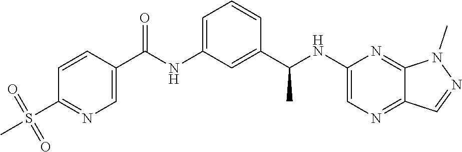 [SMILES: C[C@H](Nc1cnc2cnn(C)c2n1)c1cccc(NC(=O)c2ccc(S(C)(=O)=O)nc2)c1] | (S)-N-(3-(1-((1-methyl-1H- pyrazolo[3,4-b]pyrazin-6-yl) amino)ethyl)phenyl)-6- (methylsulfonyl)nicotinamide |
| 222 | 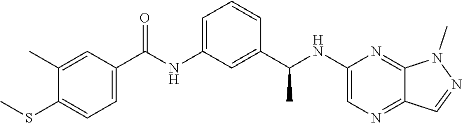 [SMILES: CSc1ccc(C(=O)Nc2cccc([C@H](C)Nc3cnc4cnn(C)c4n3)c2)cc1C] | (S)-3-methyl-N-(3-(1-((1- methyl-1H-pyrazolo[3,4-b] pyrazin-6-yl)amino)ethyl) phenyl)-4- (methylthio)benzamide |
| 223 | 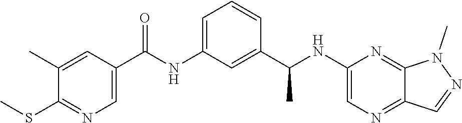 [SMILES: CSc1ncc(C(=O)Nc2cccc([C@H](C)Nc3cnc4cnn(C)c4n3)c2)cc1C] | (S)-5-methyl-N-(3-(1-((1- methyl-1H-pyrazolo[3,4-b] pyrazin-6-yl)amino)ethyl) phenyl)-6- (methylthio)nicotinamide |
| 224 | 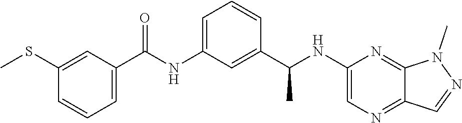 [SMILES: CSc1cccc(C(=O)Nc2cccc([C@H](C)Nc3cnc4cnn(C)c4n3)c2)c1] | (S)-N-(3-(1-((1-methyl-1H- pyrazolo[3,4-b]pyrazin-6-yl) amino)ethyl)phenyl)-3- (methylthio)benzamide |
| 225 | 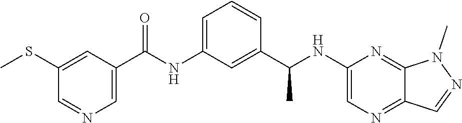 [SMILES: CSc1cncc(C(=O)Nc2cccc([C@H](C)Nc3cnc4cnn(C)c4n3)c2)c1] | (S)-N-(3-(1-((1-methyl-1H- pyrazolo[3,4-b]pyrazin-6-yl) amino)ethyl)phenyl)-5- (methylthio)benzamide |
| 226 | 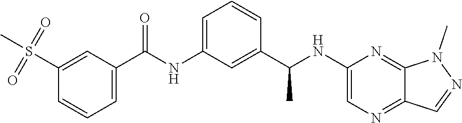 [SMILES: C[C@H](Nc1cnc2cnn(C)c2n1)c1cccc(NC(=O)c2cccc(S(C)(=O)=O)c2)c1] | (S)-N-(3-(1-((1-methyl-1H- pyrazolo[3,4-b]pyrazin-6-yl) amino)ethyl)phenyl)-3- (methylsulfonyl)benzamide |
| 227 | 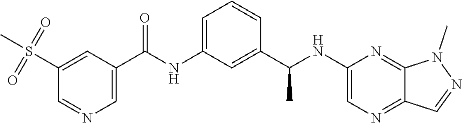 [SMILES: C[C@H](Nc1cnc2cnn(C)c2n1)c1cccc(NC(=O)c2cncc(S(C)(=O)=O)c2)c1] | (S)-N-(3-(1-((1-methyl-1H- pyrazolo[3,4-b]pyrazin-6-yl) amino)ethyl)phenyl)-5- (methylsulfonyl)nicotinamide |
| 228 | 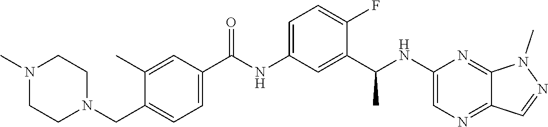 [SMILES: Cc1cc(C(=O)Nc2ccc(F)c([C@H](C)Nc3cnc4cnn(C)c4n3)c2)ccc1CN1CCN(C)CC1] | (S)-N-(4-fluoro-3-(1-((1-methyl- 1H-pyrazolo[3,4-b]pyrazin-6-yl) amino)ethyl)phenyl)-3-methyl- 4-((4-methylpiperazin-1-yl) methyl)benzamide |
| 229 | 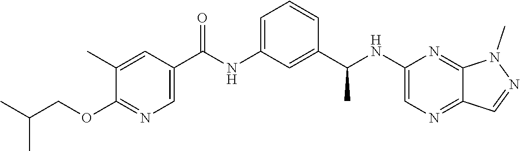 [SMILES: Cc1cc(C(=O)Nc2cccc([C@H](C)Nc3cnc4cnn(C)c4n3)c2)cnc1OCC(C)C] | (S)-6-isobutoxy-5-methyl-N-(3- (1-((1-methyl-1H-pyrazolo[3,4- b]pyrazin-6-yl)amino)ethyl) phenyl)nicotinamide |
| 230 | 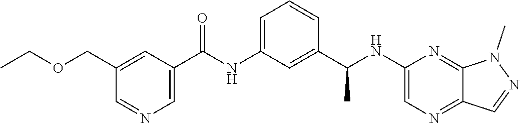 [SMILES: CCOCc1cncc(C(=O)Nc2cccc([C@H](C)Nc3cnc4cnn(C)c4n3)c2)c1] | (S)-5-(ethoxymethyl)-N-(3-(1- ((1-methyl-1H-pyrazolo[3,4-b] pyrazin-6-yl)amino)ethyl) phenyl)nicotinamide |
| 231 | 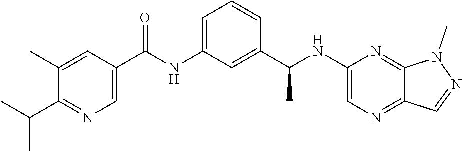 [SMILES: Cc1cc(C(=O)Nc2cccc([C@H](C)Nc3cnc4cnn(C)c4n3)c2)cnc1C(C)C] | (S)-6-isopropyl-5-methyl-N-(3- (1-((1-methyl-1H-pyrazolo[3,4- 6]pyrazin-6-yl)amino)ethyl) phenyl)nicotinamide |
| 232 | 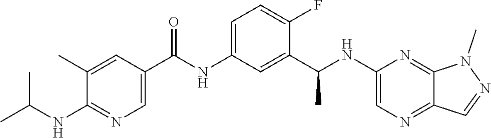 [SMILES: Cc1cc(C(=O)Nc2ccc(F)c([C@H](C)Nc3cnc4cnn(C)c4n3)c2)cnc1NC(C)C] | (S)-N-(4-fluoro-3-(1-((1-methyl- 1H-pyrazolo[3,4-b]pyrazin-6-yl) amino)ethyl)phenyl)-6- (isopropylamino)-5- methylnicotinamide |
| 233 | 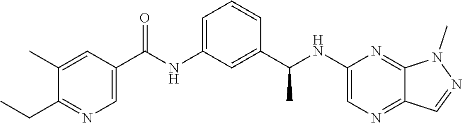 [SMILES: CCc1ncc(C(=O)Nc2cccc([C@H](C)Nc3cnc4cnn(C)c4n3)c2)cc1C] | (S)-6-ethyl-5-methyl-N-(3-(1- ((1-methyl-1H-pyrazolo[3,4-b] pyrazin-6-yl)amino)ethyl) phenyl)nicotinamide |
| 234 | 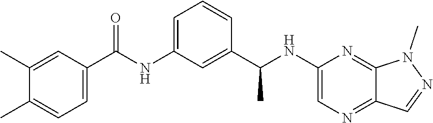 [SMILES: Cc1ccc(C(=O)Nc2cccc([C@H](C)Nc3cnc4cnn(C)c4n3)c2)cc1C] | (S)-3,4-dimethyl-N-(3-(1-((1- methyl-1H-pyrazolo[3,4-b] pyrazin-6-yl)amino)ethyl) phenyl)benzamide |
| 235 | 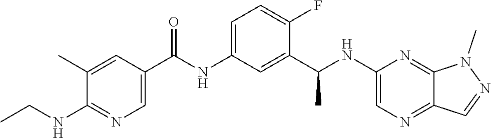 [SMILES: CCNc1ncc(C(=O)Nc2ccc(F)c([C@H](C)Nc3cnc4cnn(C)c4n3)c2)cc1C] | (S)-6-(ethylamino)-N-(4-fluoro- 3-(1-((1-methyl-1H- pyrazolo[3,4-b]pyrazin-6-yl) amino)ethyl)phenyl)-5- methylnicotinamide |
| 236 | 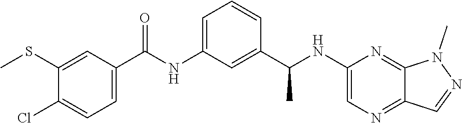 [SMILES: CSc1cc(C(=O)Nc2cccc([C@H](C)Nc3cnc4cnn(C)c4n3)c2)ccc1Cl] | (S)-4-chloro-N-(3-(1-((1- methyl-1H-pyrazolo[3,4-b] pyrazin-6-yl)amino)ethyl) phenyl)-3- (methylthio)benzamide |
| 237 | 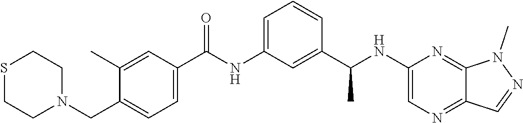 [SMILES: Cc1cc(C(=O)Nc2cccc([C@H](C)Nc3cnc4cnn(C)c4n3)c2)ccc1CN1CCSCC1] | (S)-3-methyl-N-(3-(1-((1- methyl-1H-pyrazolo[3,4-b] pyrazin-6-yl)amino)ethyl) phenyl)-4- (thiomorpholinomethyl) benzamide |
| 238 | 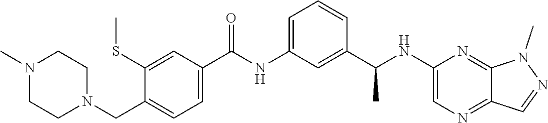 [SMILES: CSc1cc(C(=O)Nc2cccc([C@H](C)Nc3cnc4cnn(C)c4n3)c2)ccc1CN1CCN(C)CC1] | (S)-N-(3-(1-((1-methyl-1H- pyrazolo[3,4-b]pyrazin-6-yl) amino)ethyl)phenyl)-4-((4- methylpiperazin-1-yl)methyl)-3- (methylthio)benzamide |
| 239 | 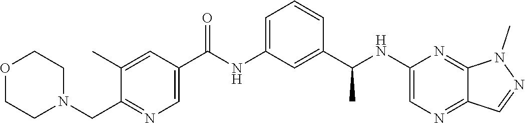 [SMILES: Cc1cc(C(=O)Nc2cccc([C@H](C)Nc3cnc4cnn(C)c4n3)c2)cnc1CN1CCOCC1] | (S)-5-methyl-N-(3-(1-((1- methyl-1H-pyrazolo[3,4-b] pyrazin-6-yl)amino)ethyl) phenyl)-6-(morpholinomethyl) nicotinamide |
| 240 | 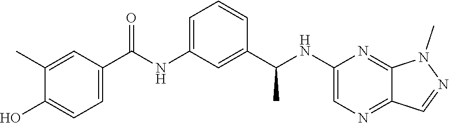 [SMILES: Cc1cc(C(=O)Nc2cccc([C@H](C)Nc3cnc4cnn(C)c4n3)c2)ccc1O] | (S)-4-hydroxy-3-methyl-N-(3- (1-((1-methylH-pyrazolo[3,4- b]pyrazin-6-yl)amino)ethyl) phenyl)benzamide |
| 241 | 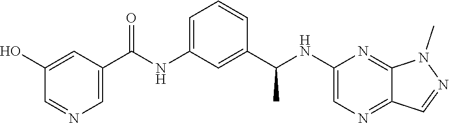 [SMILES: C[C@H](Nc1cnc2cnn(C)c2n1)c1cccc(NC(=O)c2cncc(O)c2)c1] | (S)-5-hydroxy-N-(3-(1-((1- methyl-1H-pyrazolo[3,4-b] pyrazin-6-yl)amino)ethyl) phenyl)nicotinamide |
| 242 | 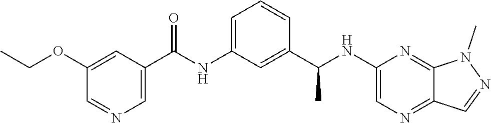 [SMILES: CCOc1cncc(C(=O)Nc2cccc([C@H](C)Nc3cnc4cnn(C)c4n3)c2)c1] | (S)-5-ethoxy-N-(3-(1-((1- methyl-1H-pyrazolo[3,4-b] pyrazin-6-yl)amino)ethyl) phenyl)nicotinamide |
| 243 | 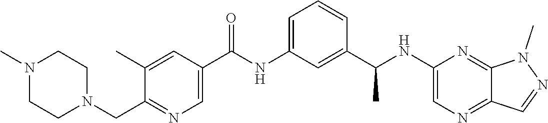 [SMILES: Cc1cc(C(=O)Nc2cccc([C@H](C)Nc3cnc4cnn(C)c4n3)c2)cnc1CN1CCN(C)CC1] | (S)-5-methyl-N-(3-(1-((1- methyl-1H-pyrazolo[3,4-b] pyrazin-6-yl)amino)ethyl) phenyl)-6-((4-methylpiperazin- 1-yl)methyl)nicotinamide |
| 244 | 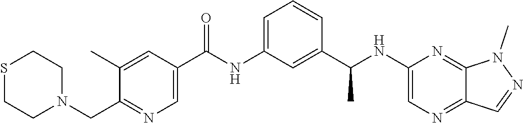 [SMILES: Cc1cc(C(=O)Nc2cccc([C@H](C)Nc3cnc4cnn(C)c4n3)c2)cnc1CN1CCSCC1] | (S)-5-methyl-N-(3-(1-((1- methyl-1H-pyrazolo[3,4-b] pyrazin-6-yl)amino)ethyl) phenyl)-6- (thiomorpholinomethyl) nicotinamide |
| 245 | 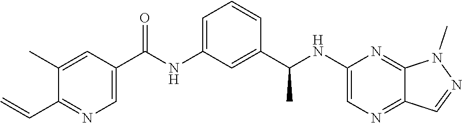 [SMILES: C=Cc1ncc(C(=O)Nc2cccc([C@H](C)Nc3cnc4cnn(C)c4n3)c2)cc1C] | (S)-5-methyl-N-(3-(1-((1- methyl-1H-pyrazolo[3,4-b] pyrazin-6-yl)amino)ethyl) phenyl)-6-vinylnicotinamide |
| 246 | 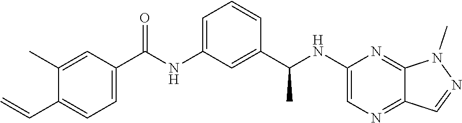 [SMILES: C=Cc1ccc(C(=O)Nc2cccc([C@H](C)Nc3cnc4cnn(C)c4n3)c2)cc1C] | (S)-3-methyl-N-(3-(1-((1- methyl-1H-pyrazolo[3,4-b] pyrazin-6-yl)amino)ethyl) phenyl)-4-vinylbenzamide |
| 247 | 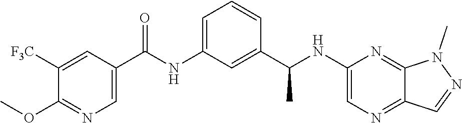 [SMILES: COc1ncc(C(=O)Nc2cccc([C@H](C)Nc3cnc4cnn(C)c4n3)c2)cc1C(F)(F)F] | (S)-6-methoxy-N-(3-(1-((1- methyl-1-pyrazolo[3,4-b] pyrazin-6-yl)amino)ethyl) phenyl)-5-(trifluoromethyl) nicotinamide |
| 248 | 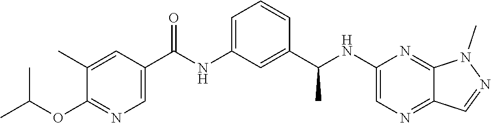 [SMILES: Cc1cc(C(=O)Nc2cccc([C@H](C)Nc3cnc4cnn(C)c4n3)c2)cnc1OC(C)C] | (S)-6-isopropoxy-5-methyl-N- (3-(1-((1-methyl-1H- pyrazolo[3,4-b]pyrazin-6-yl) amino)ethyl)phenyl) nicotinamide |
| 249 | 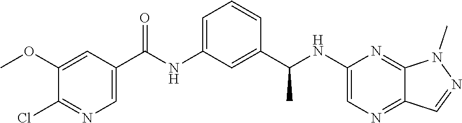 [SMILES: COc1cc(C(=O)Nc2cccc([C@H](C)Nc3cnc4cnn(C)c4n3)c2)cnc1Cl] | (S)-6-chloro-5-methoxy-N-(3- (1-((1-methyl-1H-pyrazolo[3,4- b]pyrazin-6-yl)amino)ethyl) phenyl)nicotinamide |
| 250 | 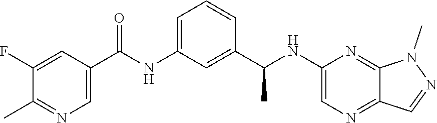 [SMILES: Cc1ncc(C(=O)Nc2cccc([C@H](C)Nc3cnc4cnn(C)c4n3)c2)cc1F] | (S)-5-fluoro-6-methyl-N-(3-(1- ((1-methyl-1H-pyrazolo[3,4-b] pyrazin-6-yl)amino)ethyl) phenyl)nicotinamide |
| 251 | 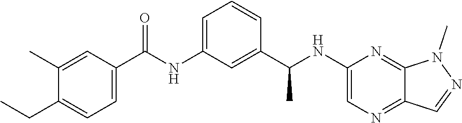 [SMILES: CCc1ccc(C(=O)Nc2cccc([C@H](C)Nc3cnc4cnn(C)c4n3)c2)cc1C] | (S)-4-ethyl-3-methyl-N-(3-(1- ((1-methyl-1H-pyrazolo[3,4-b] pyrazin-6-yl)amino)ethyl) phenyl)benzamide |
| 252 | 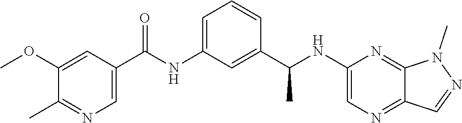 [SMILES: COc1cc(C(=O)Nc2cccc([C@H](C)Nc3cnc4cnn(C)c4n3)c2)cnc1C] | (S)-5-methoxy-6-methyl-N-(3- (1-((1-methyl-1H-pyrazolo[3,4- b]pyrazin-6-yl)amino)ethyl) phenyl)nicotinamide |
| 253 | 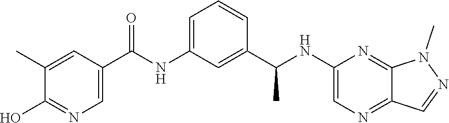 [SMILES: Cc1cc(C(=O)Nc2cccc([C@H](C)Nc3cnc4cnn(C)c4n3)c2)cnc1O] | (S)-6-hydroxy-5-methyl-N-(3- (1-((1-methyl-1H-pyrazolo[3,4- b]pyrazin-6-yl)amino)ethyl) phenyl)nicotinamide |
| 254 | 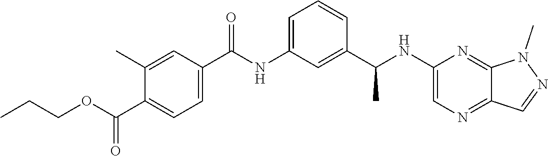 [SMILES: CCCOC(=O)c1ccc(C(=O)Nc2cccc([C@H](C)Nc3cnc4cnn(C)c4n3)c2)cc1C] | propyl (S)-2-methyl-4-((3-(1- ((1-methyl-1H-pyrazolo[3,4-b] pyrazin-6-yl)amino)ethyl) phenyl)carbamoyl)benzoate |
| 255 | 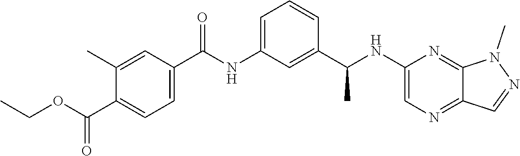 [SMILES: CCOC(=O)c1ccc(C(=O)Nc2cccc([C@H](C)Nc3cnc4cnn(C)c4n3)c2)cc1C] | ethyl (S)-2-methyl-4-((3-(1-((1- methyl-1H-pyrazolo[3,4-b] pyrazin-6-yl)amino)ethyl) phenyl)carbamoyl)benzoate |
| 256 | 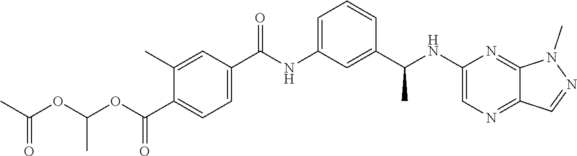 [SMILES: CC(=O)OC(C)OC(=O)c1ccc(C(=O)Nc2cccc([C@H](C)Nc3cnc4cnn(C)c4n3)c2)cc1C] | 1-acetoxyethyl 2-methyl-4-((3- ((S)-1-((1-methyl-1H- pyrazolo[3,4-b]pyrazin-6-yl) amino)ethyl)phenyl)carbamoyl) benzoate |
| 257 | 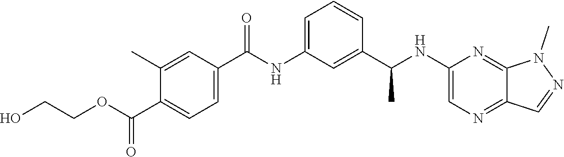 [SMILES: Cc1cc(C(=O)Nc2cccc([C@H](C)Nc3cnc4cnn(C)c4n3)c2)ccc1C(=O)OCCO] | 2-hydroxyethyl (S)-2-methyl-4- ((3-(1-((1-methyl-1H- pyrazolo[3,4-b]pyrazin-6-yl) amino)ethyl)phenyl)carbamoyl) benzoate |
| 258 | 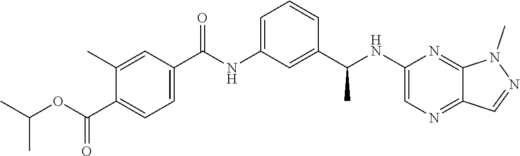 [SMILES: Cc1cc(C(=O)Nc2cccc([C@H](C)Nc3cnc4cnn(C)c4n3)c2)ccc1C(=O)OC(C)C] | isopropyl (S)-2-methyl-4-((3-(1- ((1-methyl-1H-pyrazolo[3,4-b] pyrazin-6-yl)amino)ethyl) phenyl)carbamoyl)benzoate |
| 259 | 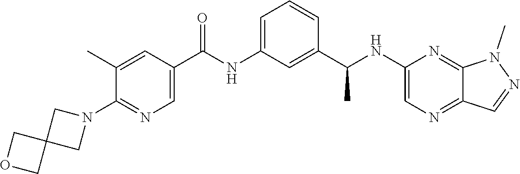 [SMILES: Cc1cc(C(=O)Nc2cccc([C@H](C)Nc3cnc4cnn(C)c4n3)c2)cnc1N1CC2(COC2)C1] | (S)-5-methyl-N-(3-(1-((1- methyl-1H-pyrazolo[3,4-b] pyrazin-6-yl)amino)ethyl) phenyl)-6-(2-oxa-6- azaspiro[3.3]heptan-6-yl) nicotinamide |
| 260 | 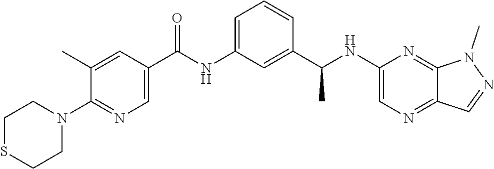 [SMILES: Cc1cc(C(=O)Nc2cccc([C@H](C)Nc3cnc4cnn(C)c4n3)c2)cnc1N1CCSCC1] | (S)-5-methyl-N-(3-(1-((1- methyl-1H-pyrazolo[3,4-b] pyrazin-6-yl)amino)ethyl) phenyl)-6- thiomorpholinonicotinamide |
| 261 | 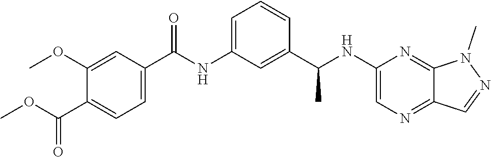 [SMILES: COC(=O)c1ccc(C(=O)Nc2cccc([C@H](C)Nc3cnc4cnn(C)c4n3)c2)cc1OC] | methyl (S)-2-methoxy-4-((3-(1- ((1-methyl-1H-pyrazolo[3,4-b] pyrazin-6-yl)amino)ethyl) phenyl)carbamoyl)benzoate |
| 262 | 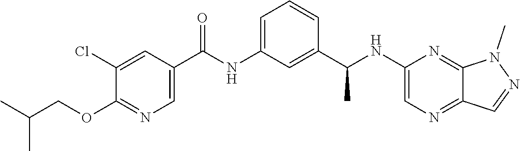 [SMILES: CC(C)COc1ncc(C(=O)Nc2cccc([C@H](C)Nc3cnc4cnn(C)c4n3)c2)cc1Cl] | (S)-5-chloro-6-isobutoxy-N-(3- (1-((1-methyl-1H-pyrazolo[3,4- b]pyrazin-6-yl)amino)ethyl) phenyl)nicotinamide |
| 263 | 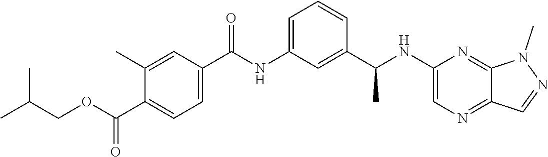 [SMILES: Cc1cc(C(=O)Nc2cccc([C@H](C)Nc3cnc4cnn(C)c4n3)c2)ccc1C(=O)OCC(C)C] | isobutyl (S)-2-methyl-4-((3-(1- ((1-methyl-1H-pyrazolo[3,4-b] pyrazin-6-yl)amino)ethyl) phenyl)carbamoyl)benzoate |
| 264 | 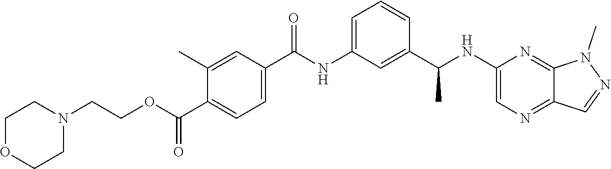 [SMILES: Cc1cc(C(=O)Nc2cccc([C@H](C)Nc3cnc4cnn(C)c4n3)c2)ccc1C(=O)OCCN1CCOCC1] | 2-morpholinoethyl (S)-2- methyl-4-((3-(1-((1-methyl-1H- pyrazolo[3,4-b]pyrazin-6-yl) amino)ethyl)phenyl)carbamoyl) benzoate |
| 265 | 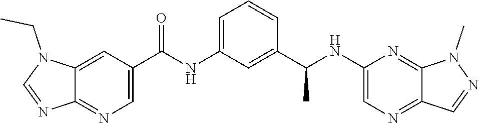 [SMILES: CCn1cnc2ncc(C(=O)Nc3cccc([C@H](C)Nc4cnc5cnn(C)c5n4)c3)cc21] | (S)-1-ethyl-N-(3-(1-((1-methyl- 1H-pyrazolo[3,4-b]pyrazin-6-yl) amino)ethyl)phenyl)-1H- imidazo[4,5-b]pyridine-6- carboxamide |
| 266 | 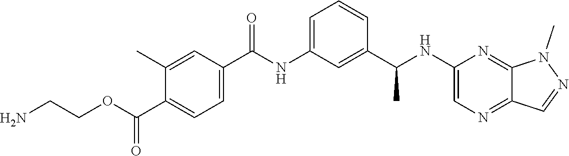 [SMILES: Cc1cc(C(=O)Nc2cccc([C@H](C)Nc3cnc4cnn(C)c4n3)c2)ccc1C(=O)OCCN] | 2-aminoethyl (S)-2-methyl-4- ((3-(1-((1-methyl-1H- pyrazolo[3,4-b]pyrazin-6-yl) amino)ethyl)phenyl)carbamoyl) benzoate |
| 267 | 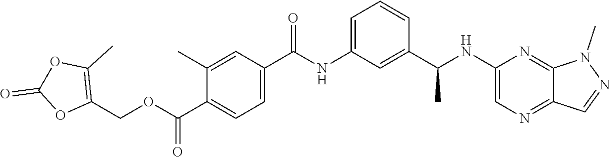 [SMILES: Cc1cc(C(=O)Nc2cccc([C@H](C)Nc3cnc4cnn(C)c4n3)c2)ccc1C(=O)OCc1oc(=O)oc1C] | (5-methyl-2-oxo-1,3-dioxol-4- yl)methyl (S)-2-methyl-4-((3-(1- ((1-methyl-1H-pyrazolo[3,4-b] pyrazin-6-yl)amino)ethyl) phenyl)carbamoyl)benzoate |
| 268 | 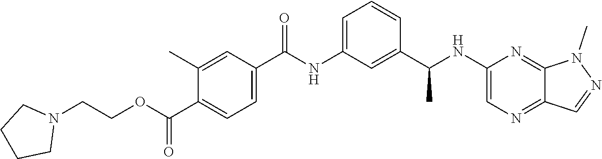 [SMILES: Cc1cc(C(=O)Nc2cccc([C@H](C)Nc3cnc4cnn(C)c4n3)c2)ccc1C(=O)OCCN1CCCC1] | 2-(pyrrolidin-1-yl)ethyl (S)-2- methyl-4-((3-(1-((1-methyl-1H- pyrazolo[3,4-b]pyrazin-6-yl) amino)ethyl)phenyl)carbamoyl) benzoate |
| 269 | 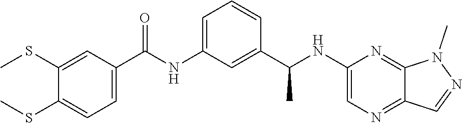 [SMILES: CSc1ccc(C(=O)Nc2cccc([C@H](C)Nc3cnc4cnn(C)c4n3)c2)cc1SC] | (S)-N-(3-(1-((1-methyl-1H- pyrazolo[3,4-b]pyrazin-6-yl) amino)ethyl)phenyl)-3,4- bis(methylthio)benzamide |
| 270 | 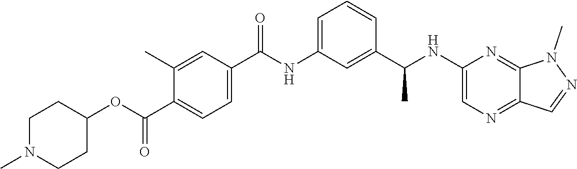 [SMILES: Cc1cc(C(=O)Nc2cccc([C@H](C)Nc3cnc4cnn(C)c4n3)c2)ccc1C(=O)OC1CCN(C)CC1] | 1-methylpiperidin-4-yl (S)-2- methyl-4-((3-(1-((1-methyl-1H- pyrazolo[3,4-b]pyrazin-6-yl) amino)ethyl)phenyl)carbamoyl) benzoate |
| 271 | 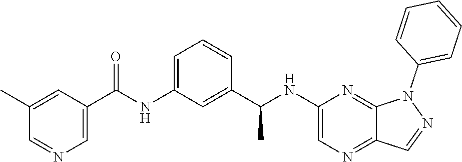 [SMILES: Cc1cncc(C(=O)Nc2cccc([C@H](C)Nc3cnc4cnn(-c5ccccc5)c4n3)c2)c1] | (S)-5-methyl-N-(3-(1-((1- phenyl-1H-pyrazolo[3,4-b] pyrazin-6-yl)amino)ethyl) phenyl)nicotinamide |
| 272 | 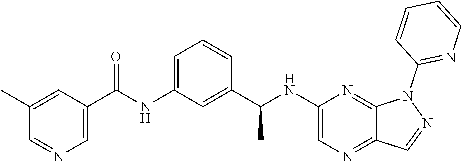 [SMILES: Cc1cncc(C(=O)Nc2cccc([C@H](C)Nc3cnc4cnn(-c5ccccn5)c4n3)c2)c1] | (S)-5-methyl-N-(3-(1-((1- (pyridin-2-yl)-1H-pyrazolo[3,4- b]pyrazin-6-yl)amino)ethyl) phenyl)nicotinamide |
| 273 | 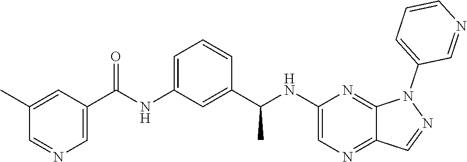 [SMILES: Cc1cncc(C(=O)Nc2cccc([C@H](C)Nc3cnc4cnn(-c5cccnc5)c4n3)c2)c1] | (S)-5-methyl-N-(3-(1-((1- (pyridin-3-yl)-1H-pyrazolo[3,4- b]pyrazin-6-yl)amino)ethyl) phenyl)nicotinamide |
| 274 | 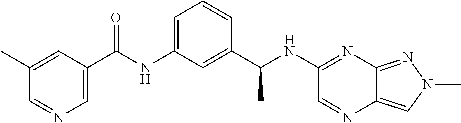 | (S)-5-methyl-N-(3-(1-((2- methyl-2H-pyrazolo[3,4-b] pyrazin-6-yl)amino)ethyl) phenyl)nicotinamide |
| 275 | 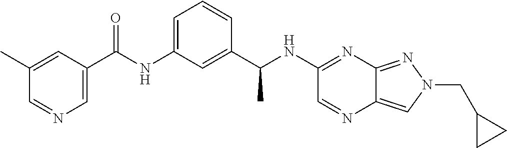 [SMILES: Cc1cncc(C(=O)Nc2cccc([C@H](C)Nc3cnc4cn(CC5CC5)nc4n3)c2)c1] | (S)-N-(3-(1-((2- (cyclopropylmethyl)-2H- pyrazolo[3,4-b]pyrazin-6-yl) amino)ethyl)phenyl)-5- methylnicotinamide |
| 276 | 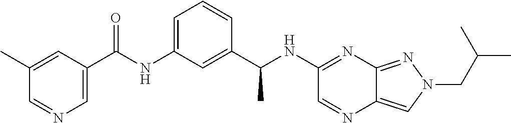 [SMILES: Cc1cncc(C(=O)Nc2cccc([C@H](C)Nc3cnc4cn(CC(C)C)nc4n3)c2)c1] | (S)-N-(3-(1-((2-isobutyl-2H- pyrazolo[3,4-b]pyrazin-6-yl) amino)ethyl)phenyl)-5- methylnicotinamide |
| 277 | 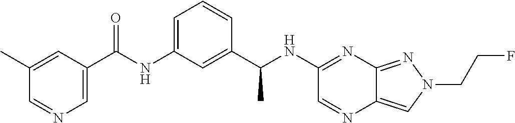 [SMILES: Cc1cncc(C(=O)Nc2cccc([C@H](C)Nc3cnc4cn(CCF)nc4n3)c2)c1] | (S)-N-(3-(1-((2-(2-fluoroethyl)- 2H-pyrazolo[3,4-b]pyrazin-6-yl) amino)ethyl)phenyl)-5- methylnicotinamide |
| 278 | 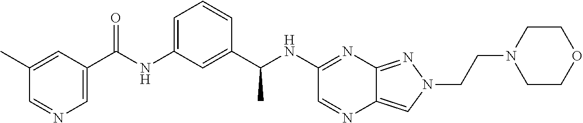 [SMILES: Cc1cncc(C(=O)Nc2cccc([C@H](C)Nc3cnc4cn(CCN5CCOCC5)nc4n3)c2)c1] | (S)-5-methyl-N-(3-(1-((2-(2- morpholinoethyl)-2H- pyrazolo[3,4-b]pyrazin-6-yl) amino)ethyl)phenyl) nicotinamide |
| 279 | 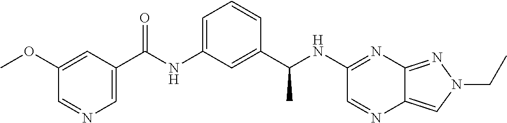 [SMILES: CCn1cc2ncc(N[C@@H](C)c3cccc(NC(=O)c4cncc(OC)c4)c3)nc2n1] | (S)-N-(3-(1-((2-ethyl-2H- pyrazolo[3,4-b]pyrazin-6-yl) amino)ethyl)phenyl)-5- methoxynicotinamide |
| 280 | 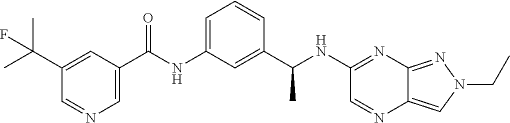 [SMILES: CCn1cc2ncc(N[C@@H](C)c3cccc(NC(=O)c4cncc(C(C)(C)F)c4)c3)nc2n1] | (S)-N-(3-(1-((2-ethyl-2H- pyrazolo[3,4-b]pyrazin-6-yl) amino)ethyl)phenyl)-5-(2- fluoropropan-2-yl)nicotinamide |
| 281 | 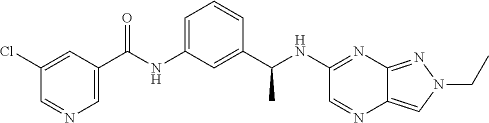 [SMILES: CCn1cc2ncc(N[C@@H](C)c3cccc(NC(=O)c4cncc(Cl)c4)c3)nc2n1] | (S)-5-chloro-N-(3-(1-((2-ethyl- 2H-pyrazolo[3,4-b]pyrazin-6-yl) amino)ethyl)phenyl) nicotinamide |
| 282 | 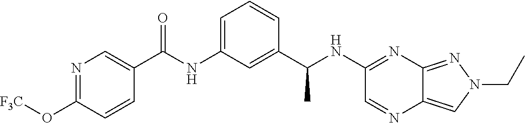 [SMILES: CCn1cc2ncc(N[C@@H](C)c3cccc(NC(=O)c4ccc(OC(F)(F)F)nc4)c3)nc2n1] | (S)-N-(3-(1-((2-ethyl-2H- pyrazolo[3,4-b]pyrazin-6-yl) amino)ethyl)phenyl)-6- (trifluoromethoxy)nicotinamide |
| 283 | 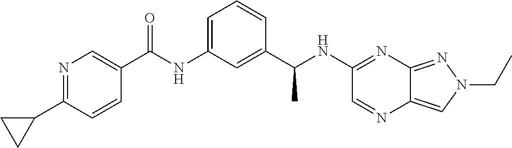 [SMILES: CCn1cc2ncc(N[C@@H](C)c3cccc(NC(=O)c4ccc(C5CC5)nc4)c3)nc2n1] | (S)-6-cyclopropyl-N-(3-(1-((2- ethyl-2H-pyrazolo[3,4-b] pyrazin-6-yl)amino)ethyl) phenyl)nicotinamide |
| 284 | 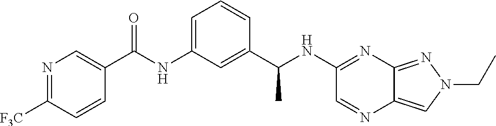 [SMILES: CCn1cc2ncc(N[C@@H](C)c3cccc(NC(=O)c4ccc(C(F)(F)F)nc4)c3)nc2n1] | (S)-N-(3-(1-((2-ethyl-2H- pyrazolo[3,4-b]pyrazin-6-yl) amino)ethyl)phenyl)-6- (trifluoromethyl)nicotinamide |
| 285 | 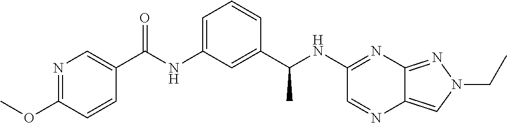 [SMILES: CCn1cc2ncc(N[C@@H](C)c3cccc(NC(=O)c4ccc(OC)nc4)c3)nc2n1] | (S)-N-(3-(1-((2-ethyl-2H- pyrazolo[3,4-b]pyrazin-6-yl) amino)ethyl)phenyl)-6- methoxynicotinamide |
| 286 | 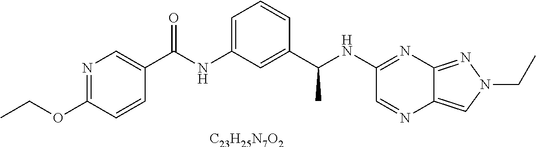 [SMILES: CCOc1ccc(C(=O)Nc2cccc([C@H](C)Nc3cnc4cn(CC)nc4n3)c2)cn1 ; "C23H25N7O2"] | (S)-6-ethoxy-N-(3-(1-((2-ethyl- 2H-pyrazolo[3,4-b]pyrazin-6-yl) amino)ethyl)phenyl) nicotinamide |
| 287 | 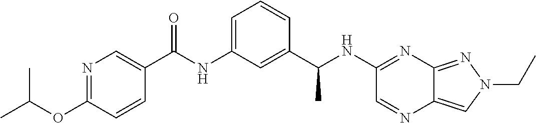 [SMILES: CCn1cc2ncc(N[C@@H](C)c3cccc(NC(=O)c4ccc(OC(C)C)nc4)c3)nc2n1] | (S)-N-(3-(1-((2-ethyl-2H- pyrazolo[3,4-b]pyrazin-6-yl) amino)ethyl)phenyl)-6- isopropoxynicotinamide |
| 288 | 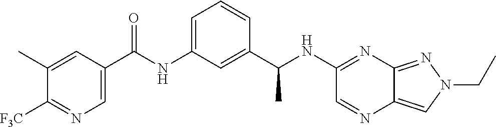 [SMILES: CCn1cc2ncc(N[C@@H](C)c3cccc(NC(=O)c4cnc(C(F)(F)F)c(C)c4)c3)nc2n1] | (S)-N-(3-(1-((2-ethyl-2H- pyrazolo[3,4-b]pyrazin-6-yl) amino)ethyl)phenyl)-5-methyl- 6-(trifluoromethyl)nicotinamide |
| 289 | 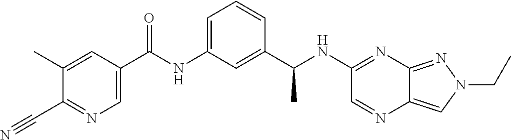 [SMILES: CCn1cc2ncc(N[C@@H](C)c3cccc(NC(=O)c4cnc(C#N)c(C)c4)c3)nc2n1] | (S)-6-cyano-N-(3-(1-((2-ethyl- 2H-pyrazolo[3,4-b]pyrazin-6-yl) amino)ethyl)phenyl)-5- methylnicotinamide |
| 290 | 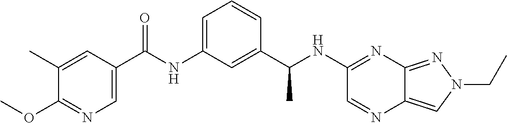 [SMILES: CCn1cc2ncc(N[C@@H](C)c3cccc(NC(=O)c4cnc(OC)c(C)c4)c3)nc2n1] | (S)-N-(3-(1-((2-ethyl-2H- pyrazolo[3,4-b]pyrazin-6-yl) amino)ethyl)phenyl)-6- methoxy-5-methylnicotinamide |
| 291 | 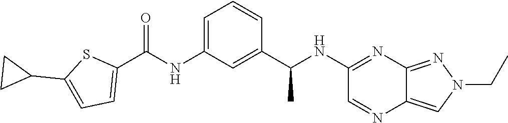 [SMILES: CCn1cc2ncc(N[C@@H](C)c3cccc(NC(=O)c4ccc(C5CC5)s4)c3)nc2n1] | (S)-5-cyclopropyl-N-(3-(1-((2- ethyl-2H-pyrazolo[3,4-b] pyrazin-6-yl)amino)ethyl) phenyl)thiophene-2- carboxamide |
| 292 | 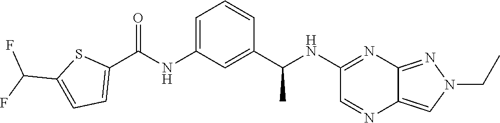 [SMILES: CCn1cc2ncc(N[C@@H](C)c3cccc(NC(=O)c4ccc(C(F)F)s4)c3)nc2n1] | (S)-5-(difluoromethyl)-N-(3-(1- ((2-ethyl-2H-pyrazolo[3,4-b] pyrazin-6-yl)amino)ethyl) phenyl)thiophene-2- carboxamide |
| 293 | 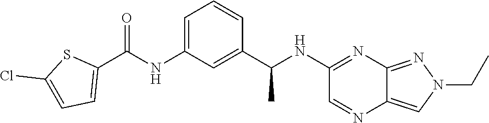 [SMILES: CCn1cc2ncc(N[C@@H](C)c3cccc(NC(=O)c4ccc(Cl)s4)c3)nc2n1] | (S)-5-chloro-N-(3-(1-((2-ethyl- 2H-pyrazolo[3,4-b]pyrazin-6-yl) amino)ethyl)phenyl)thiophene- 2-carboxamide |
| 294 | 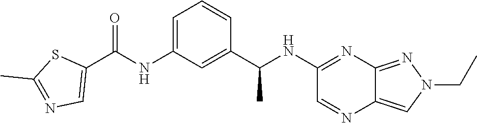 [SMILES: CCn1cc2ncc(N[C@@H](C)c3cccc(NC(=O)c4cnc(C)s4)c3)nc2n1] | (S)-N-(3-(1-((2-ethyl-2H- pyrazolo[3,4-b]pyrazin-6-yl) amino)ethyl)phenyl)-2- methylthiazole-5-carboxamide |
| 295 | 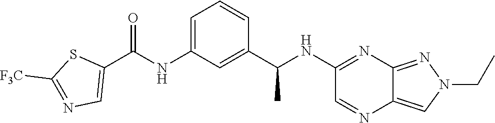 [SMILES: CCn1cc2ncc(N[C@@H](C)c3cccc(NC(=O)c4cnc(C(F)(F)F)s4)c3)nc2n1] | (S)-N-(3-(1-((2-ethyl-2H- pyrazolo[3,4-b]pyrazin-6-yl) amino)ethyl)phenyl)-2- (trifluoromethyl)thiazole-5- carboxamide |
| 296 | 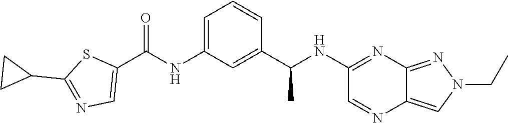 [SMILES: CCn1cc2ncc(N[C@@H](C)c3cccc(NC(=O)c4cnc(C5CC5)s4)c3)nc2n1] | (S)-2-cyclopropyl-N-(3-(1-((2- ethyl-2H-pyrazolo[3,4-b] pyrazin-6-yl)amino)ethyl) phenyl)thiazole-5-carboxamide |
| 297 | 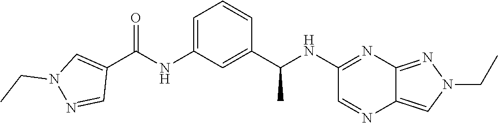 [SMILES: CCn1cc(C(=O)Nc2cccc([C@H](C)Nc3cnc4cn(CC)nc4n3)c2)cn1] | (S)-1-ethyl-N-(3-(1-((2-ethyl- 2H-pyrazolo[3,4-b]pyrazin-6-yl) amino)ethyl)phenyl)-1H- pyrazole-4-carboxamide |
| 298 | 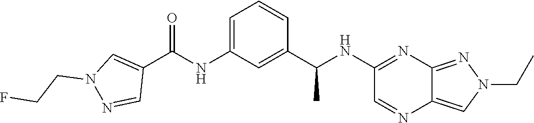 [SMILES: CCn1cc2ncc(N[C@@H](C)c3cccc(NC(=O)c4cnn(CCF)c4)c3)nc2n1] | (S)-N-(3-(1-((2-ethyl-2H- pyrazolo[3,4-b]pyrazin-6-yl) amino)ethyl)phenyl)-1-(2- fluoroethyl)-1H-pyrazole-4- carboxamide |
| 299 | 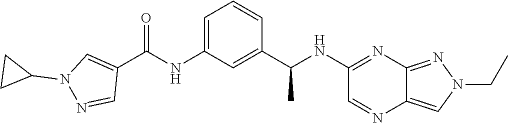 [SMILES: CCn1cc2ncc(N[C@@H](C)c3cccc(NC(=O)c4cnn(C5CC5)c4)c3)nc2n1] | (S)-1-cyclopropyl-N-(3-(1-((2- ethyl-2H-pyrazolo[3,4-b] pyrazin-6-yl)amino)ethyl) phenyl)-1H-pyrazole-4- carboxamide |
| 300 | 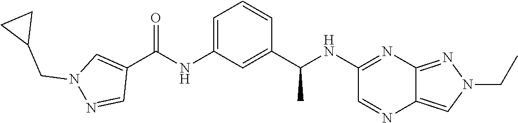 [SMILES: CCn1cc2ncc(N[C@@H](C)c3cccc(NC(=O)c4cnn(CC5CC5)c4)c3)nc2n1] | (S)-1-(cyclopropylmethyl)-N-(3- (1-((2-ethyl-2H-pyrazolo[3,4-b] pyrazin-6-yl)amino)ethyl) phenyl)-1H-pyrazole-4- carboxamide |
| 301 | 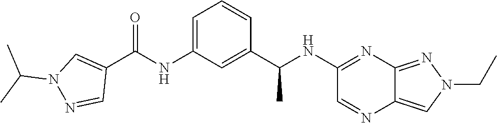 [SMILES: CCn1cc2ncc(N[C@@H](C)c3cccc(NC(=O)c4cnn(C(C)C)c4)c3)nc2n1] | (S)-N-(3-(1-((2-ethyl-2H- pyrazolo[3,4-b]pyrazin-6-yl) amino)ethyl)phenyl)-1- isopropyl-1H-pyrazole-4- carboxamide |
| 302 | 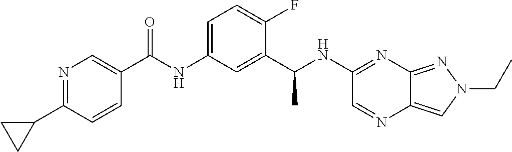 [SMILES: CCn1cc2ncc(N[C@@H](C)c3cc(NC(=O)c4ccc(C5CC5)nc4)ccc3F)nc2n1] | (S)-6-cyclopropyl-N-(3-(1-((2- ethyl-2H-pyrazolo[3,4-b] pyrazin-6-yl)amino)ethyl)-4- fluorophenyl)nicotinamide |
| 303 | 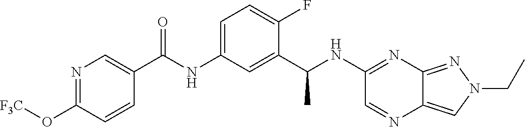 [SMILES: CCn1cc2ncc(N[C@@H](C)c3cc(NC(=O)c4ccc(OC(F)(F)F)nc4)ccc3F)nc2n1] | (S)-N-(3-(1-((2-ethyl-2H- pyrazolo[3,4-b]pyrazin-6-yl) amino)ethyl)-4-fluorophenyl)-6- (trifluoromethoxy)nicotinamide |
| 304 | 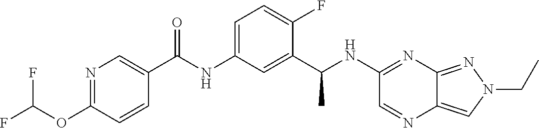 [SMILES: CCn1cc2ncc(N[C@@H](C)c3cc(NC(=O)c4ccc(OC(F)F)nc4)ccc3F)nc2n1] | (S)-6-(difluoromethoxy)-N-(3- (1-((2-ethyl-2H-pyrazolo[3,4-b] pyrazin-6-yl)amino)ethyl)-4- fluorophenyl)nicotinamide |
| 305 | 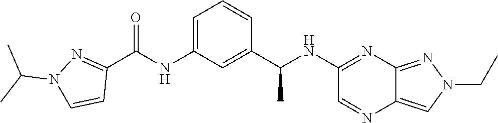 [SMILES: CCn1cc2ncc(N[C@@H](C)c3cccc(NC(=O)c4ccn(C(C)C)n4)c3)nc2n1] | (S)-N-(3-(1-((2-ethyl-2H- pyrazolo[3,4-b]pyrazin-6-yl) amino)ethyl)phenyl)-1- isopropyl-1H-pyrazole-3- carboxamide |
| 306 | 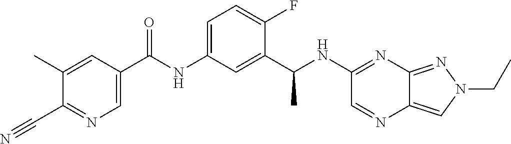 [SMILES: CCn1cc2ncc(N[C@@H](C)c3cc(NC(=O)c4cnc(C#N)c(C)c4)ccc3F)nc2n1] | (S)-6-cyano-N-(3-(1-((2-ethyl- 2H-pyrazolo[3,4-b]pyrazin-6-yl) amino)ethyl)-4-fluorophenyl)-5- methylnicotinamide |
| 307 | 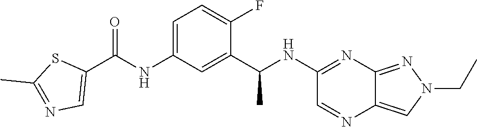 [SMILES: CCn1cc2ncc(N[C@@H](C)c3cc(NC(=O)c4cnc(C)s4)ccc3F)nc2n1] | (S)-N-(3-(1-((2-ethyl-2H- pyrazolo[3,4-b]pyrazin-6-yl) amino)ethyl)-4-fluorophenyl)-2- methylthiazole-5-carboxamide |
| 308 | 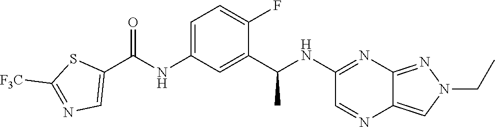 [SMILES: CCn1cc2ncc(N[C@@H](C)c3cc(NC(=O)c4cnc(C(F)(F)F)s4)ccc3F)nc2n1] | (S)-N-(3-(1-((2-ethyl-2H- pyrazolo[3,4-b]pyrazin-6-yl) amino)ethyl)-4-fluorophenyl)-2- (trifluoromethyl)thiazole-5- carboxamide |
| 309 | 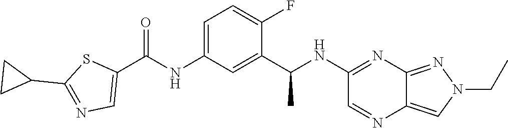 [SMILES: CCn1cc2ncc(N[C@@H](C)c3cc(NC(=O)c4cnc(C5CC5)s4)ccc3F)nc2n1] | (S)-2-cyclopropyl-N-(3-(1-((2- ethyl-2H-pyrazolo[3,4-b] pyrazin-6-yl)amino)ethyl)-4- fluorophenyl)thiazole-5- carboxamide |
| 310 | 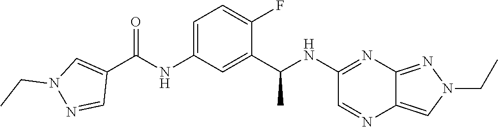 [SMILES: CCn1cc(C(=O)Nc2ccc(F)c([C@H](C)Nc3cnc4cn(CC)nc4n3)c2)cn1] | (S)-1-ethyl-N-(3-(1-((2-ethyl- 2H-pyrazolo[3,4-b]pyrazin-6-yl) amino)ethyl)-4-fluorophenyl)- 1H-pyrazole-4-carboxamide |
| 311 | 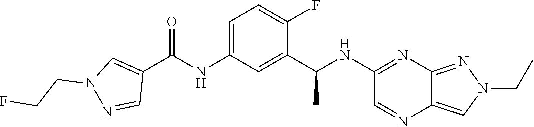 [SMILES: CCn1cc2ncc(N[C@@H](C)c3cc(NC(=O)c4cnn(CCF)c4)ccc3F)nc2n1] | (S)-N-(3-(1-((2-ethyl-2H- pyrazolo[3,4-b]pyrazin-6-yl) amino)ethyl)-4-fluorophenyl)-1- (2-fluoroethyl)-1H-pyrazole-4- carboxamide |
| 312 | 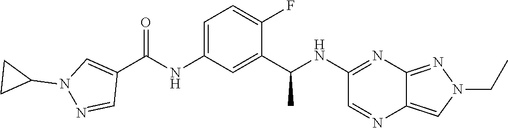 [SMILES: CCn1cc2ncc(N[C@@H](C)c3cc(NC(=O)c4cnn(C5CC5)c4)ccc3F)nc2n1] | (S)-1-cyclopropyl-N-(3-(1-((2- ethyl-2H-pyrazolo[3,4-b] pyrazin-6-yl)amino)ethyl)-4- fluorophenyl)-1H-pyrazole-4- carboxamide |
| 313 | 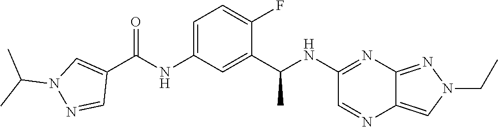 [SMILES: CCn1cc2ncc(N[C@@H](C)c3cc(NC(=O)c4cnn(C(C)C)c4)ccc3F)nc2n1] | (S)-N-(3-(1-((2-ethyl-2H- pyrazolo[3,4-b]pyrazin-6-yl) amino)ethyl)-4-fluorophenyl)-1- isopropyl-1H-pyrazole-4- carboxamide |
| 314 | 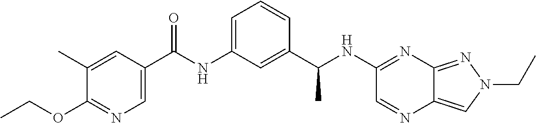 [SMILES: CCOc1ncc(C(=O)Nc2cccc([C@H](C)Nc3cnc4cn(CC)nc4n3)c2)cc1C] | (S)-6-ethoxyN-(3-(1-((2-ethyl- 2H-pyrazolo[3,4-b]pyrazin-6-yl) amino)ethyl)phenyl)-5- methylnicotinamide |
| 315 | 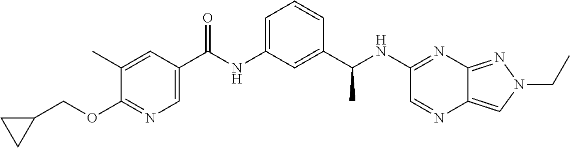 [SMILES: CCn1cc2ncc(N[C@@H](C)c3cccc(NC(=O)c4cnc(OCC5CC5)c(C)c4)c3)nc2n1] | (S)-6-(cyclopropylmethoxy)-N- (3-(1-((2-ethyl-2H-pyrazolo[3,4- b]pyrazin-6-yl)amino)ethyl) phenyl)-5-methylnicotinamide |
| 316 | 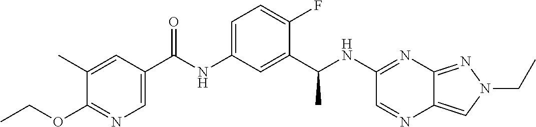 [SMILES: CCOc1ncc(C(=O)Nc2ccc(F)c([C@H](C)Nc3cnc4cn(CC)nc4n3)c2)cc1C] | (S)-6-ethoxy-N-(3-(1-((2-ethyl- 2H-pyrazolo[3,4-b]pyrazin-6-yl) amino)ethyl)-4-fluorophenyl)-5- methylnicotinamide |
| 317 | 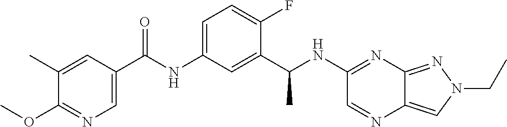 [SMILES: CCn1cc2ncc(N[C@@H](C)c3cc(NC(=O)c4cnc(OC)c(C)c4)ccc3F)nc2n1] | (S)-N-(3-(1-((2-ethyl-2H- pyrazolo[3,4-b]pyrazin-6-yl) amino)ethyl)-4-fluorophenyl)-6- methoxy-5-methylnicotinamide |
| 318 | 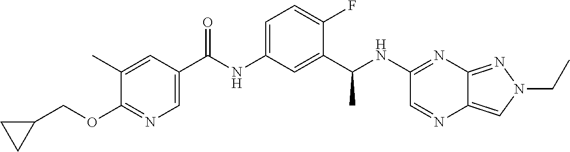 [SMILES: CCn1cc2ncc(N[C@@H](C)c3cc(NC(=O)c4cnc(OCC5CC5)c(C)c4)ccc3F)nc2n1] | (S)-6-(cyclopropylmethoxy)-N- (3-(1-((2-ethyl-2H-pyrazolo[3,4- b]pyrazin-6-yl)amino)ethyl)-4- fluorophenyl)-5- methylnicotinamide |
| 319 | 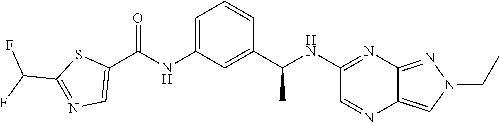 [SMILES: CCn1cc2ncc(N[C@@H](C)c3cccc(NC(=O)c4cnc(C(F)F)s4)c3)nc2n1] | (S)-2-(difluoromethyl)-N-(3-(1- ((2-ethyl-2H-pyrazolo[3,4-b] pyrazin-6-yl)amino)ethyl) phenyl)thiazole-5-carboxamide |
| 320 | 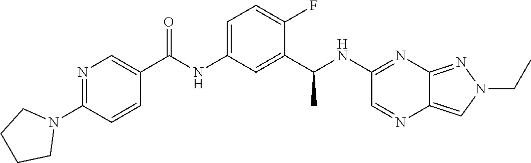 [SMILES: CCn1cc2ncc(N[C@@H](C)c3cc(NC(=O)c4ccc(N5CCCC5)nc4)ccc3F)nc2n1] | (S)-N-(3-(1-((2-ethyl-2H- pyrazolo[3,4-b]pyrazin-6-yl) amino)ethyl)-4-fluorophenyl)-6- (pyrrolidin-1-yl)nicotinamide |
| 321 | 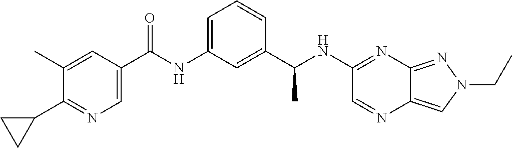 [SMILES: CCn1cc2ncc(N[C@@H](C)c3cccc(NC(=O)c4cnc(C5CC5)c(C)c4)c3)nc2n1] | (S)-6-cyclopropyl-N-(3-(1-((2- ethyl-2H-pyrazolo[3,4-b] pyrazin-6-yl)amino)ethyl) phenyl)-5-methylnicotinamide |
| 322 | 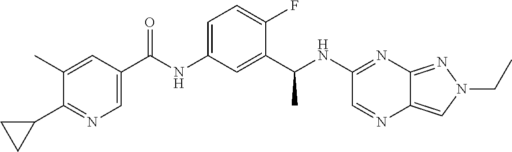 [SMILES: CCn1cc2ncc(N[C@@H](C)c3cc(NC(=O)c4cnc(C5CC5)c(C)c4)ccc3F)nc2n1] | (S)-6-cyclopropyl-N-(3-(1-((2- ethyl-2H-pyrazolo[3,4-b] pyrazin-6-yl)amino)ethyl)-4- fluorophenyl)-5- methylnicotinamide |
| 323 | 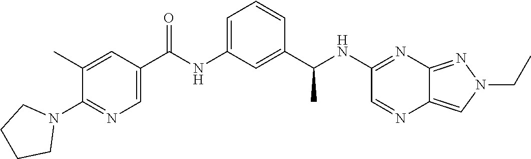 [SMILES: CCn1cc2ncc(N[C@@H](C)c3cccc(NC(=O)c4cnc(N5CCCC5)c(C)c4)c3)nc2n1] | (S)-N-(3-(1-((2-ethyl-2H- pyrazolo[3,4-b]pyrazin-6-yl) amino)ethyl)phenyl)-5-methyl- 6-(pyrrolidin-1-yl)nicotinamide |
| 324 | 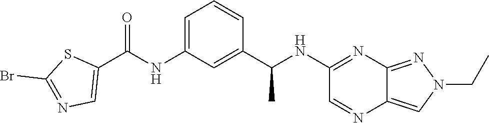 [SMILES: CCn1cc2ncc(N[C@@H](C)c3cccc(NC(=O)c4cnc(Br)s4)c3)nc2n1] | (S)-2-bromo-N-(3-(1-((2-ethyl- 2H-pyrazolo[3,4-b]pyrazin-6-yl) amino)ethyl)phenyl)thiazole-5- carboxamide |
| 325 | 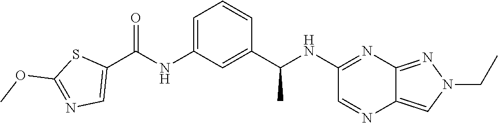 [SMILES: CCn1cc2ncc(N[C@@H](C)c3cccc(NC(=O)c4cnc(OC)s4)c3)nc2n1] | (S)-N-( 3-(1-((2-ethyl-2H- pyrazolo[3,4-b]pyrazin-6-yl) amino)ethyl)phenyl)-2- methoxythiazole-5-carboxamide |
| 326 | 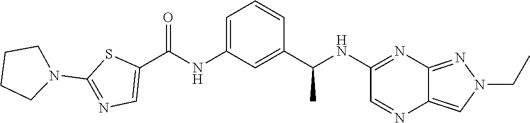 [SMILES: CCn1cc2ncc(N[C@@H](C)c3cccc(NC(=O)c4cnc(N5CCCC5)s4)c3)nc2n1] | (S)-N-(3-(1-((2-ethyl-2H- pyrazolo[3,4-b]pyrazin-6-yl) amino)ethyl)phenyl)-2- (pyrrolidin-1-yl)thiazole-5- carboxamide |
| 327 | 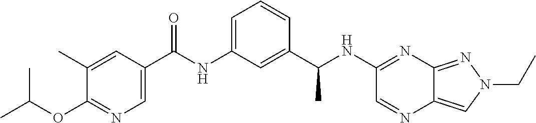 [SMILES: CCn1cc2ncc(N[C@@H](C)c3cccc(NC(=O)c4cnc(OC(C)C)c(C)c4)c3)nc2n1] | (S)-N-(3-(1-((2-ethyl-2H- pyrazolo[3,4-b]pyrazin-6-yl) amino)ethyl)phenyl)-6- isopropoxy-5- methylnicotinamide |
| 328 | 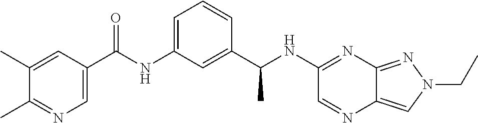 [SMILES: CCn1cc2ncc(N[C@@H](C)c3cccc(NC(=O)c4cnc(C)c(C)c4)c3)nc2n1] | (S)-N-(3-(1-((2-ethyl-2H- pyrazolo[3,4-b]pyrazin-6-yl) amino)ethyl)phenyl)-5,6- dimethylnicotinamide |
| 329 | 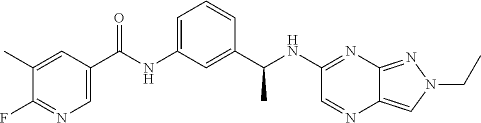 [SMILES: CCn1cc2ncc(N[C@@H](C)c3cccc(NC(=O)c4cnc(F)c(C)c4)c3)nc2n1] | (S)-N-(3-(1-((2-ethyl-2H- pyrazolo[3,4-b]pyrazin-6-yl) amino)ethyl)phenyl)-6-fluoro-5- methylnicotinamide |
| 330 | 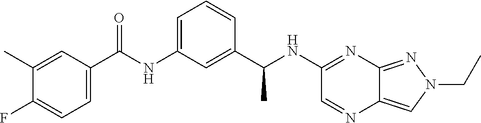 [SMILES: CCn1cc2ncc(N[C@@H](C)c3cccc(NC(=O)c4ccc(F)c(C)c4)c3)nc2n1] | (S)-N-(3-(1-((2-ethyl-2H- pyrazolo[3,4-b]pyrazin-6-yl) amino)ethyl)phenyl)-4-fluoro-3- methylbenzamide |
| 331 | 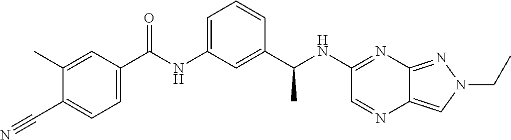 [SMILES: CCn1cc2ncc(N[C@@H](C)c3cccc(NC(=O)c4ccc(C#N)c(C)c4)c3)nc2n1] | (S)-4-cyano-N-(3-(1-((2-ethyl- 2H-pyrazolo[3,4-b]pyrazin-6-yl) amino)ethyl)phenyl)-3- methylbenzamide |
| 332 | 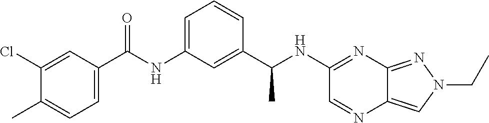 [SMILES: CCn1cc2ncc(N[C@@H](C)c3cccc(NC(=O)c4ccc(C)c(Cl)c4)c3)nc2n1] | (S)-3-chloro-N-(3-(1-((2-ethyl- 2H-pyrazolo[3,4-b]pyrazin-6-yl) amino)ethyl)phenyl)-4- methylbenzamide |
| 333 | 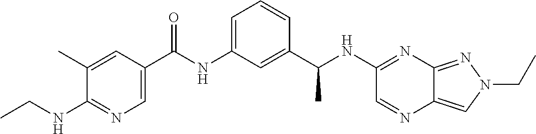 [SMILES: CCNc1ncc(C(=O)Nc2cccc([C@H](C)Nc3cnc4cn(CC)nc4n3)c2)cc1C] | (S)-N-(3-(1-((2-ethyl-2H- pyrazolo[3,4-b]pyrazin-6-yl) amino)ethyl)phenyl)-6- (ethylamino)-5- methylnicotinamide |
| 334 | 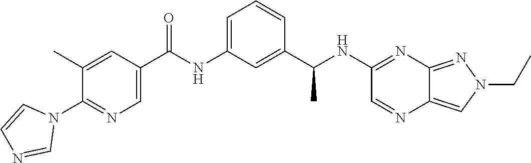 [SMILES: CCn1cc2ncc(N[C@@H](C)c3cccc(NC(=O)c4cnc(-n5ccnc5)c(C)c4)c3)nc2n1] | (S)-N-(3-(1-((2-ethyl-2H- pyrazolo[3,4-b]pyrazin-6-yl) amino)ethyl)phenyl)-6-(1H- imidazol-1-yl)-5- methylnicotinamide |
| 335 | 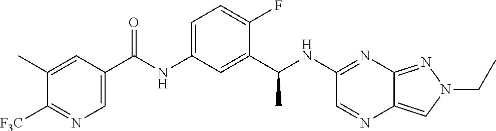 [SMILES: CCn1cc2ncc(N[C@@H](C)c3cc(NC(=O)c4cnc(C(F)(F)F)c(C)c4)ccc3F)nc2n1] | (S)-N-(3-(1-((2-ethyl-2H- pyrazolo[3,4-b]pyrazin-6-yl) amino)ethyl)-4-fluorophenyl)-5- methyl-6-(trifluoromethyl) nicotinamide |
| 336 | 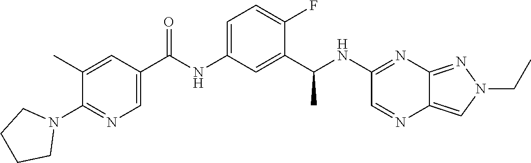 [SMILES: CCn1cc2ncc(N[C@@H](C)c3cc(NC(=O)c4cnc(N5CCCC5)c(C)c4)ccc3F)nc2n1] | (S)-N-(3-(1-((2-ethyl-2H- pyrazolo[3,4-b]pyrazin-6-yl) amino)ethyl)-4-fluorophenyl)-5- methyl-6-(pyrrolidin-1-yl) nicotinamide |
| 337 | 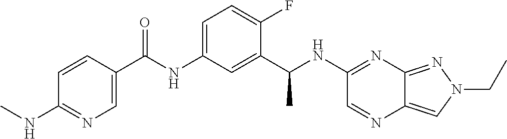 [SMILES: CCn1cc2ncc(N[C@@H](C)c3cc(NC(=O)c4ccc(NC)nc4)ccc3F)nc2n1] | (S)-N-(3-(1-((2-ethyl-2H- pyrazolo[3,4-b]pyrazin-6-yl) amino)ethyl)-4-fluorophenyl)-6- (methylamino)nicotinamide |
| 338 | 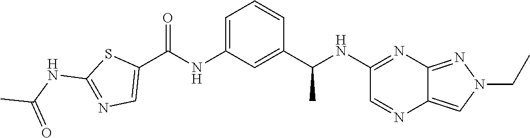 [SMILES: CCn1cc2ncc(N[C@@H](C)c3cccc(NC(=O)c4cnc(NC(C)=O)s4)c3)nc2n1] | (S)-N-(3-(1-((2-ethyl-2H- pyrazolo[3,4-b]pyrazin-6-yl) amino)ethyl)phenyl)-2- (methoxymethyl)thiazole-5- carboxamide |
| 339 | 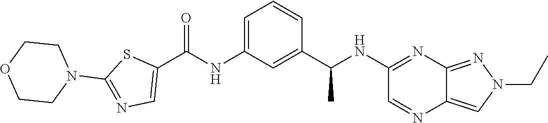 [SMILES: CCn1cc2ncc(N[C@@H](C)c3cccc(NC(=O)c4cnc(N5CCOCC5)s4)c3)nc2n1] | (S)-N-(3-(1-((2-ethyl-2H- pyrazolo[3,4-b]pyrazin-6-yl) amino)ethyl)phenyl)-2- morpholinothiazole-5- carboxamide |
| 340 | 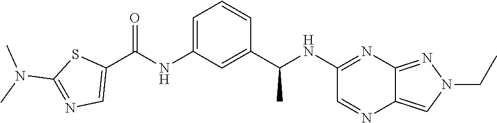 [SMILES: CCn1cc2ncc(N[C@@H](C)c3cccc(NC(=O)c4cnc(N(C)C)s4)c3)nc2n1] | (S)-2-(dimethylamino)-N-(3-(1- ((2-ethyl-2H-pyrazolo[3,4-b] pyrazin-6-yl)amino)ethyl) phenyl)thiazole-5-carboxamide |
| 341 | 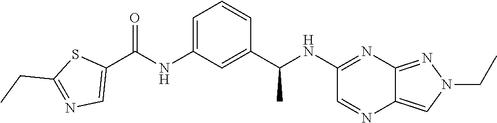 [SMILES: CCc1ncc(C(=O)Nc2cccc([C@H](C)Nc3cnc4cn(CC)nc4n3)c2)s1] | (S)-2-ethyl-N-(3-(1-((2-ethyl- 2H-pyrazolo[3,4-b]pyrazin-6-yl) amino)ethyl)phenyl)thiazole-5- carboxamide |
| 342 | 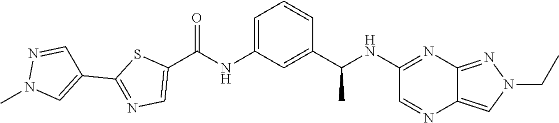 [SMILES: CCn1cc2ncc(N[C@@H](C)c3cccc(NC(=O)c4cnc(-c5cnn(C)c5)s4)c3)nc2n1] | (S)-N-(3-(1-((2-ethyl-2H- pyrazolo[3,4-b]pyrazin-6-yl) amino)ethyl)phenyl)-2-(1- methyl-1H-pyrazol-4-yl) thiazole-5-carboxamide |
| 343 | 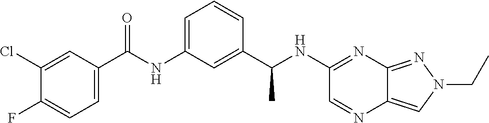 [SMILES: CCn1cc2ncc(N[C@@H](C)c3cccc(NC(=O)c4ccc(F)c(Cl)c4)c3)nc2n1] | (S)-3-chloro-N-(3-(1-((2-ethyl- 2H-pyrazolo[3,4-b]pyrazin-6-yl) amino)ethyl)phenyl)-4- fluorobenzamide |
| 344 | 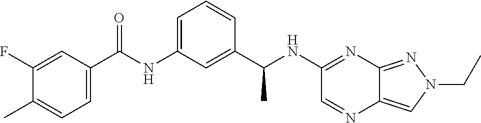 [SMILES: CCn1cc2ncc(N[C@@H](C)c3cccc(NC(=O)c4ccc(C)c(F)c4)c3)nc2n1] | (S)-N-(3-(1-((2-ethyl-2H- pyrazolo[3,4-b]pyrazin-6-yl) amino)ethyl)phenyl)-3-fluoro-4- methylbenzamide |
| 345 | 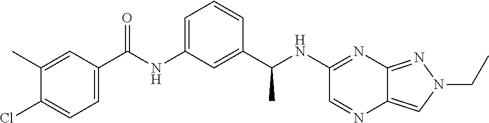 [SMILES: CCn1cc2ncc(N[C@@H](C)c3cccc(NC(=O)c4ccc(Cl)c(C)c4)c3)nc2n1] | (S)-4-chloro-N-(3-(1-((2-ethyl- 2H-pyrazolo[3,4-b]pyrazin-6-yl) amino)ethyl)phenyl)-3- methylbenzamide |
| 346 | 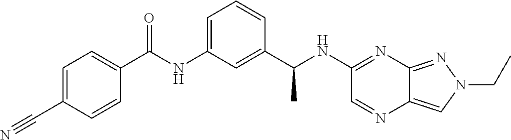 [SMILES: CCn1cc2ncc(N[C@@H](C)c3cccc(NC(=O)c4ccc(C#N)cc4)c3)nc2n1] | (S)-4-cyano-N-(3-(1-((2-ethyl- 2H-pyrazolo[3,4-b]pyrazin-6-yl) amino)ethyl)phenyl)benzamide |
| 347 | 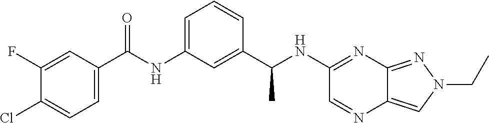 [SMILES: CCn1cc2ncc(N[C@@H](C)c3cccc(NC(=O)c4ccc(Cl)c(F)c4)c3)nc2n1] | (S)-4-chloro-N-(3-(1-((2-ethyl- 2H-pyrazolo[3,4-b]pyrazin-6-yl) amino)ethyl)phenyl)-3- fluorobenzamide |
| 348 | 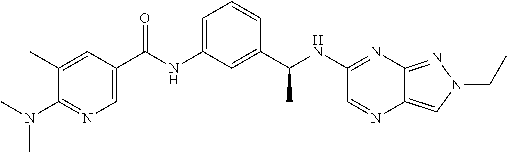 [SMILES: CCn1cc2ncc(N[C@@H](C)c3cccc(NC(=O)c4cnc(N(C)C)c(C)c4)c3)nc2n1] | (S)-6-(dimethylamino)-N-(3-(1- ((2-ethyl-2H-pyrazolo[3,4-b] pyrazin-6-yl)amino)ethyl) phenyl)-5-methylnicotinamide |
| 349 | 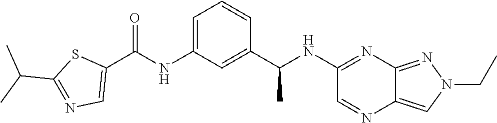 [SMILES: CCn1cc2ncc(N[C@@H](C)c3cccc(NC(=O)c4cnc(C(C)C)s4)c3)nc2n1] | (S)-N-(3-(1-((2-ethyl-2H- pyrazolo[3,4-b]pyrazin-6-yl) amino)ethyl)phenyl)-2- isopropylthiazole-5- carboxamide |
| 350 | 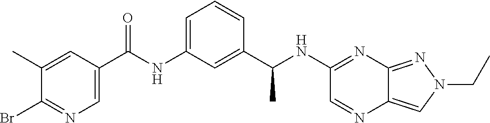 [SMILES: CCn1cc2ncc(N[C@@H](C)c3cccc(NC(=O)c4cnc(Br)c(C)c4)c3)nc2n1] | (S)-6-bromo-N-(3-(1-((2-ethyl- 2H-pyrazolo[3,4-b]pyrazin-6-yl) amino)ethyl)phenyl)-5- methylnicotinamide |
| 351 | 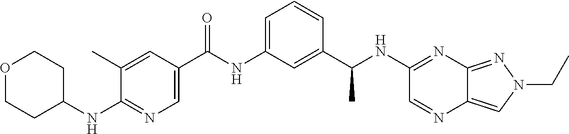 [SMILES: CCn1cc2ncc(N[C@@H](C)c3cccc(NC(=O)c4cnc(NC5CCOCC5)c(C)c4)c3)nc2n1] | (S)-N-(3-(1-((2-ethyl-2H- pyrazolo[3,4-b]pyrazin-6-yl) amino)ethyl)phenyl)-5-methyl- 6-((tetrahydro-2H-pyran-4-yl) amino)nicotinamide |
| 352 | 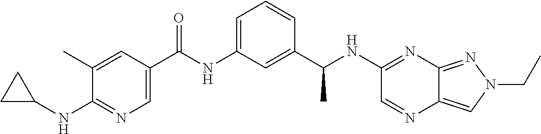 [SMILES: CCn1cc2ncc(N[C@@H](C)c3cccc(NC(=O)c4cnc(NC5CC5)c(C)c4)c3)nc2n1] | (S)-6-(cyclopropylamino)-N-(3- (1-((2-ethyl-2H-pyrazolo[3,4-b] pyrazin-6-yl)amino)ethyl) phenyl)-5-methylnicotinamide |
| 353 | 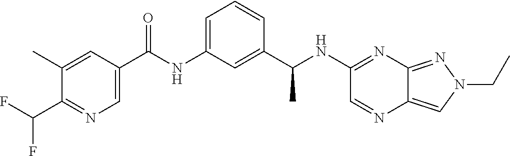 [SMILES: CCn1cc2ncc(N[C@@H](C)c3cccc(NC(=O)c4cnc(C(F)F)c(C)c4)c3)nc2n1] | (S)-6-(difluoromethyl)-N-(3-(1- ((2-ethyl-2H-pyrazolo[3,4-b)] pyrazin-6-yl)amino)ethyl) phenyl)-5-methylnicotinamide |
| 354 | 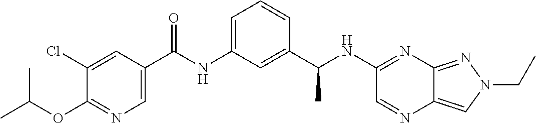 [SMILES: CCn1cc2ncc(N[C@@H](C)c3cccc(NC(=O)c4cnc(OC(C)C)c(Cl)c4)c3)nc2n1] | (S)-5-chloro-N-(3-(1-((2-ethyl- 2H-pyrazolo[3,4-b]pyrazin-6-yl) amino)ethyl)phenyl)-6- isopropoxynicotinamide |
| 355 | 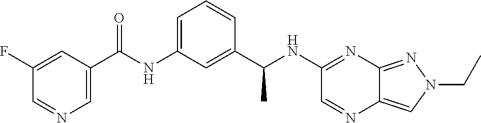 [SMILES: CCn1cc2ncc(N[C@@H](C)c3cccc(NC(=O)c4cncc(F)c4)c3)nc2n1] | (S)-N-(3-(1-((2-ethyl-2H- pyrazolo[3,4-b]pyrazin-6-yl) amino)ethyl)phenyl)-5- fluoronicotinamide |
| 356 | 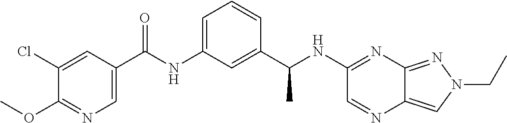 [SMILES: CCn1cc2ncc(N[C@@H](C)c3cccc(NC(=O)c4cnc(OC)c(Cl)c4)c3)nc2n1] | (S)-5-chloro-N-(3-(1-((2-ethyl- 2H-pyrazolo[3,4-b]pyrazin-6-yl) amino)ethyl)phenyl)-6- methoxynicotinamide |
| 357 | 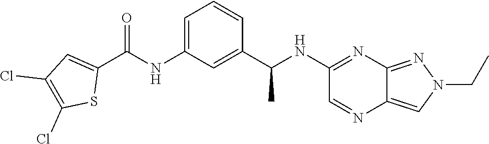 [SMILES: CCn1cc2ncc(N[C@@H](C)c3cccc(NC(=O)c4cc(Cl)c(Cl)s4)c3)nc2n1] | (S)-4,5-dichloro-N-(3-(1-((2- ethyl-2H-pyrazolo[3,4-b] pyrazin-6-yl)amino)ethyl) phenyl)thiophene-2- carboxamide |
| 358 | 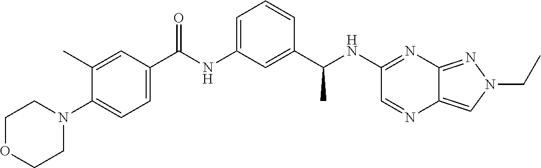 [SMILES: CCn1cc2ncc(N[C@@H](C)c3cccc(NC(=O)c4ccc(N5CCOCC5)c(C)c4)c3)nc2n1] | (S)-N-(3-(1-((2-ethyl-2H- pyrazolo[3,4-b]pyrazin-6-yl) amino)ethyl)phenyl)-5-methyl- 6-morpholinonicotinamide |
| 359 | 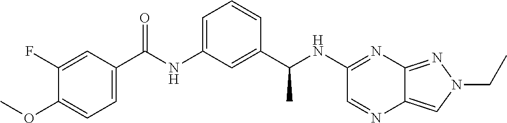 [SMILES: CCn1cc2ncc(N[C@@H](C)c3cccc(NC(=O)c4ccc(OC)c(F)c4)c3)nc2n1] | (S)-N-(3-(1-((2-ethyl-2H- pyrazolo[3,4-b]pyrazin-6-yl) amino)ethyl)phenyl)-3-fluoro-4- methoxybenzamide |
| 360 | 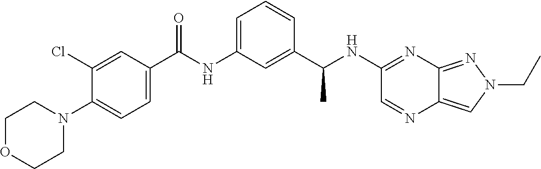 [SMILES: CCn1cc2ncc(N[C@@H](C)c3cccc(NC(=O)c4ccc(N5CCOCC5)c(Cl)c4)c3)nc2n1] | (S)-3-chloro-N-(3-(1-((2-ethyl- 2H-pyrazolo[3,4-b]pyrazin-6-yl) amino)ethyl)phenyl)-4- morpholinobenzamide |
| 361 | 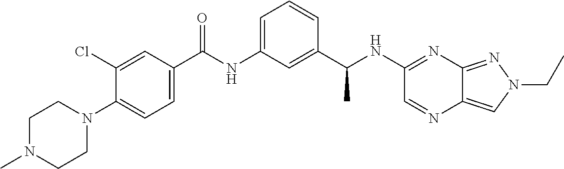 [SMILES: CCn1cc2ncc(N[C@@H](C)c3cccc(NC(=O)c4ccc(N5CCN(C)CC5)c(Cl)c4)c3)nc2n1] | (S)-3-chloro-N-(3-(1-((2-ethyl- 2H-pyrazolo[3,4-b]pyrazin-6-yl) amino)ethyl)phenyl)-4-(4- methylpiperazin-1-yl)benzamide |
| 362 | 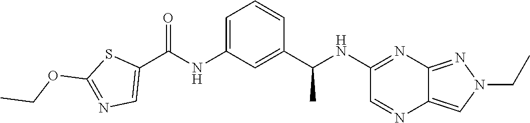 [SMILES: CCOc1ncc(C(=O)Nc2cccc([C@H](C)Nc3cnc4cn(CC)nc4n3)c2)s1] | (S)-2-ethoxy-N-(3-(1-((2-ethyl- 2H-pyrazolo[3,4-b]pyrazin-6-yl) amino)ethyl)phenyl)thiazole-5- carboxamide |
| 363 | 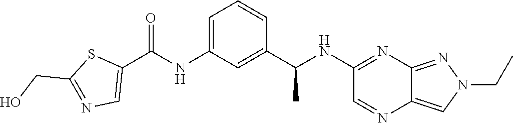 [SMILES: CCn1cc2ncc(N[C@@H](C)c3cccc(NC(=O)c4cnc(CO)s4)c3)nc2n1] | (S)-N-(3-(1-((2-ethyl-2H- pyrazolo[3,4-b]pyrazin-6-yl) amino)ethyl)phenyl)-2- (hydroxymethyl)thiazole-5- carboxamide |
| 364 | 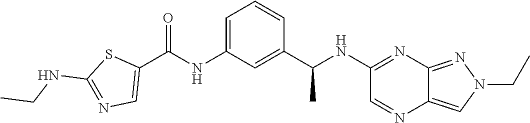 [SMILES: CCNc1ncc(C(=O)Nc2cccc([C@H](C)Nc3cnc4cn(CC)nc4n3)c2)s1] | (S)-N-(3-(1-((2-ethyl-2H- pyrazolo[3,4-b]pyrazin-6-yl) amino)ethyl)phenyl)-2- (ethylamino)thiazole-5- carboxamide |
| 365 | 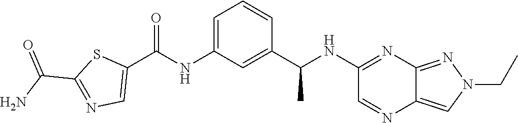 [SMILES: CCn1cc2ncc(N[C@@H](C)c3cccc(NC(=O)c4cnc(C(N)=O)s4)c3)nc2n1] | (S)-N5-(3-(1-((2-ethyl-2H- pyrazolo[3,4-b]pyrazin-6-yl) amino)ethyl)phenyl)thiazole- 2,5-dicarboxamide |
| 366 | 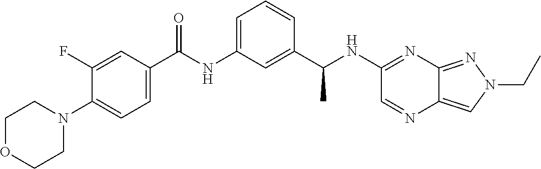 [SMILES: CCn1cc2ncc(N[C@@H](C)c3cccc(NC(=O)c4ccc(N5CCOCC5)c(F)c4)c3)nc2n1] | (S)-N-(3-(1-((2-ethyl-2H- pyrazolo[3,4-b]pyrazin-6-yl) amino)ethyl)phenyl)-3-fluoro-4- morpholinobenzamide |
| 367 | 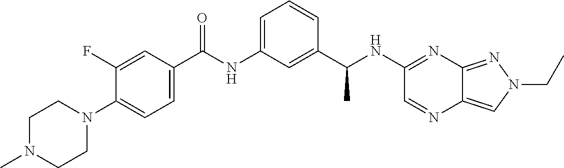 [SMILES: CCn1cc2ncc(N[C@@H](C)c3cccc(NC(=O)c4ccc(N5CCN(C)CC5)c(F)c4)c3)nc2n1] | (S)-N-(3-(1-((2-ethyl-2H- pyrazolo[3,4-b]pyrazin-6-yl) amino)ethyl)phenyl)-3-fluoro-4- (4-methylpiperazin-1-yl) benzamide |
| 368 | 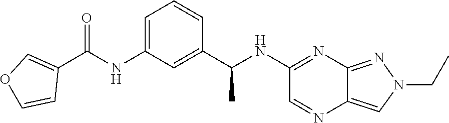 [SMILES: CCn1cc2ncc(N[C@@H](C)c3cccc(NC(=O)c4ccoc4)c3)nc2n1] | (S)-N-(3-(1-((2-ethyl-2H- pyrazolo[3,4-b]pyrazin-6-yl) amino)ethyl)phenyl)furan-3- carboxamide |
| 369 | 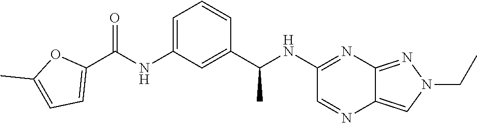 [SMILES: CCn1cc2ncc(N[C@@H](C)c3cccc(NC(=O)c4ccc(C)o4)c3)nc2n1] | (S)-N-(3-(1-((2-ethyl-2H- pyrazolo[3,4-b]pyrazin-6-yl) amino)ethyl)phenyl)-5- methylfuran-2-carboxamide |
| 370 | 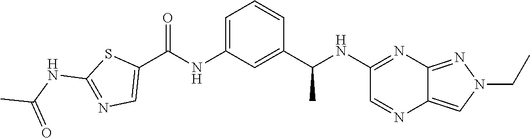 [SMILES: CCn1cc2ncc(N[C@@H](C)c3cccc(NC(=O)c4cnc(NC(C)=O)s4)c3)nc2n1] | (S)-2-acetamido-N-(3-(1-((2- ethyl-2H-pyrazolo[3,4-b] pyrazin-6-yl)amino)ethyl) phenyl)thiazole-5-carboxamide |
| 371 | 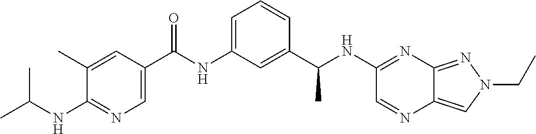 [SMILES: CCn1cc2ncc(N[C@@H](C)c3cccc(NC(=O)c4cnc(NC(C)C)c(C)c4)c3)nc2n1] | (S)-N-(3-(1-((2-ethyl-2H- pyrazolo[3,4-b]pyrazin-6-yl) amino)ethyl)phenyl)-6- (isopropylamino)-5- methylnicotinamide |
| 372 | 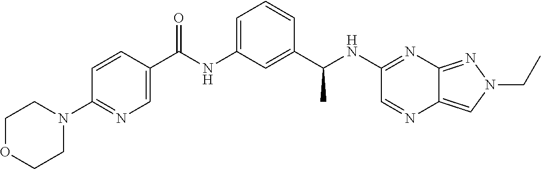 [SMILES: CCn1cc2ncc(N[C@@H](C)c3cccc(NC(=O)c4ccc(N5CCOCC5)nc4)c3)nc2n1] | (S)-N-(3-(1-((2-ethyl-2H- pyrazolo[3,4-b]pyrazin-6-yl) amino)ethyl)phenyl)-6- morpholinonicotinamide |
| 373 | 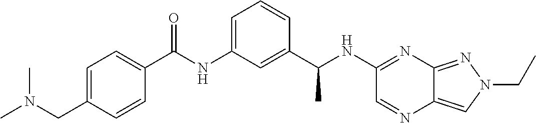 [SMILES: CCn1cc2ncc(N[C@@H](C)c3cccc(NC(=O)c4ccc(CN(C)C)cc4)c3)nc2n1] | (S)-4-((dimethylamino)methyl)- N-(3-(1-((2-ethyl-2H- pyrazolo[3,4-b]pyrazin-6-yl) amino)ethyl)phenyl)benzamide |
| 374 | 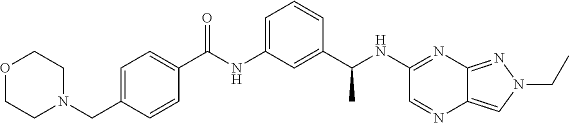 [SMILES: CCn1cc2ncc(N[C@@H](C)c3cccc(NC(=O)c4ccc(CN5CCOCC5)cc4)c3)nc2n1] | (S)-N-(3-(1-((2-ethyl-2H- pyrazolo[3,4-b]pyrazin-6-yl) amino)ethyl)phenyl)-4- (morpholinomethyl)benzamide |
| 375 | 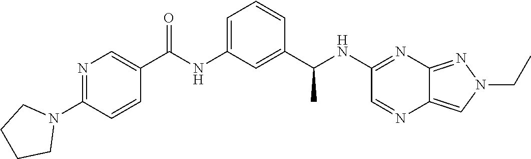 [SMILES: CCn1cc2ncc(N[C@@H](C)c3cccc(NC(=O)c4ccc(N5CCCC5)nc4)c3)nc2n1] | (S)-N-(3-(1-((2-ethyl-2H- pyrazolo[3,4-b]pyrazin-6-yl) amino)ethyl)phenyl)-6- (pyrrolidin-1-yl)nicotinamide |
| 376 | 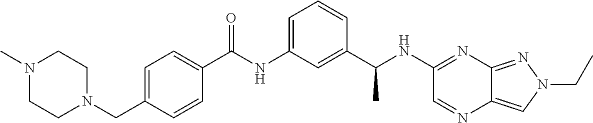 [SMILES: CCn1cc2ncc(N[C@@H](C)c3cccc(NC(=O)c4ccc(CN5CCN(C)CC5)cc4)c3)nc2n1] | (S)-N-(3-(1-((2-ethyl-2H- pyrazolo[3,4-b]pyrazin-6-yl) amino)ethyl)phenyl)-4-((4- methylpiperazin-1-yl)methyl) benzamide |
| 377 | 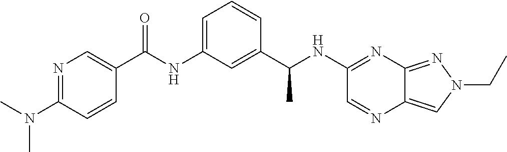 [SMILES: CCn1cc2ncc(N[C@@H](C)c3cccc(NC(=O)c4ccc(N(C)C)nc4)c3)nc2n1] | (S)-6-(dimethylamino)-N-(3-(1- ((2-ethyl-2H-pyrazolo[3,4-b] pyrazin-6-yl)amino)ethyl) phenyl)nicotinamide |
| 378 | 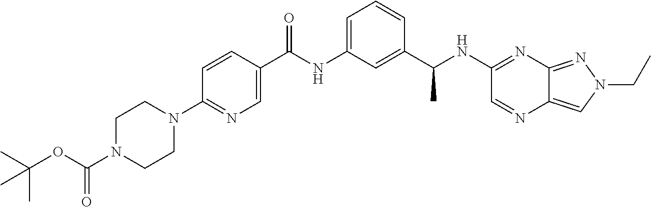 [SMILES: CCn1cc2ncc(N[C@@H](C)c3cccc(NC(=O)c4ccc(N5CCN(C(=O)OC(C)(C)C)CC5)nc4)c3)nc2n1] | tert-butyl (S)-4-(5-((3-(1-((2- ethyl-2H-pyrazolo[3,4-b] pyrazin-6-yl)amino)ethyl) phenyl)carbamoyl)pyridin-2-yl) piperazine-1-carboxylate |
| 379 | 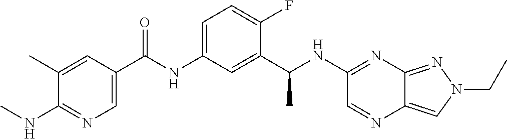 [SMILES: CCn1cc2ncc(N[C@@H](C)c3cc(NC(=O)c4cnc(NC)c(C)c4)ccc3F)nc2n1] | (S)-N-(3-(1-((2-ethyl-2H- pyrazolo[3,4-b]pyrazin-6-yl) amino)ethyl)-4-fluorophenyl)-5- methyl-6-(methylamino) nicotinamide |
| 380 | 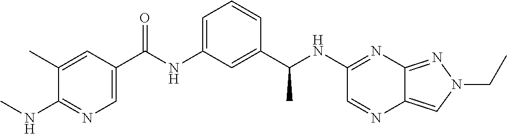 [SMILES: CCn1cc2ncc(N[C@@H](C)c3cccc(NC(=O)c4cnc(NC)c(C)c4)c3)nc2n1] | (S)-N-(3-(1-((2-ethyl-2H- pyrazolo[3,4-b]pyrazin-6-yl) amino)ethyl)phenyl)-5-methyl- 6-(methylamino)nicotinamide |
| 381 | 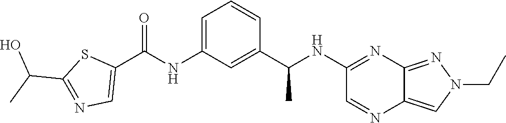 [SMILES: CCn1cc2ncc(N[C@@H](C)c3cccc(NC(=O)c4cnc(C(C)O)s4)c3)nc2n1] | N-(3-((S)-1-((2-ethyl-2H- pyrazolo[3,4-b]pyrazin-6-yl) amino)ethyl)phenyl)-2-(1- hydroxyethyl)thiazole-5- carboxamide |
| 382 | 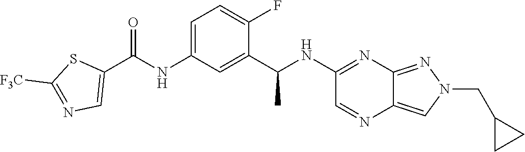 [SMILES: C[C@H](Nc1cnc2cn(CC3CC3)nc2n1)c1cc(NC(=O)c2cnc(C(F)(F)F)s2)ccc1F] | (S)-N-(3-(1-((2- (cyclopropylmethyl)-2H- pyrazolo[3,4-b]pyrazin-6-yl) amino)ethyl)-4-fluorophenyl)-2- (trifluoromethyl)thiazole-5- carboxamide |
| 383 | 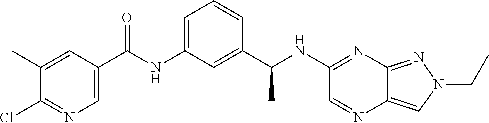 [SMILES: CCn1cc2ncc(N[C@@H](C)c3cccc(NC(=O)c4cnc(Cl)c(C)c4)c3)nc2n1] | (S)-6-chloro-N-(3-(1-((2-ethyl- 2H-pyrazolo[3,4-b]pyrazin-6-yl) amino)ethyl)phenyl)-5- methylnicotinamide |
| 384 | 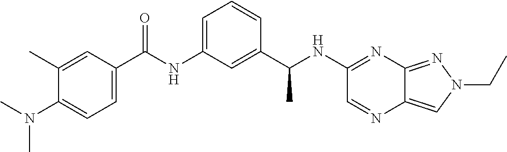 [SMILES: CCn1cc2ncc(N[C@@H](C)c3cccc(NC(=O)c4ccc(N(C)C)c(C)c4)c3)nc2n1] | (S)-4-(dimethylamino)-N-(3-(1- ((2-ethyl-2H-pyrazolo[3,4-b] pyrazin-6-yl)amino)ethyl) phenyl)-3-methylbenzamide |
| 385 | 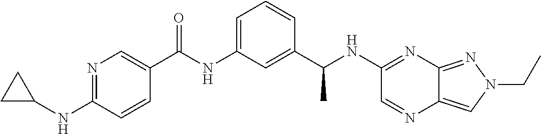 [SMILES: CCn1cc2ncc(N[C@@H](C)c3cccc(NC(=O)c4ccc(NC5CC5)nc4)c3)nc2n1] | (S)-6-(cyclopropylamino)-N-(3- (1-((2-ethyl-2H-pyrazolo[3,4-b] pyrazin-6-yl)amino)ethyl) phenyl)nicotinamide |
| 386 | 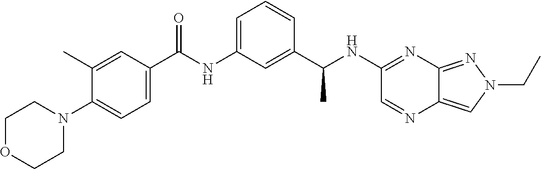 [SMILES: CCn1cc2ncc(N[C@@H](C)c3cccc(NC(=O)c4ccc(N5CCOCC5)c(C)c4)c3)nc2n1] | (S)-N-(3-(1-((2-ethyl-2H- pvrazolo[3,4-b]pyrazin-6-yl) amino)ethyl)phenyl)-3-methyl- 4-morpholinobenzamide |
| 387 | 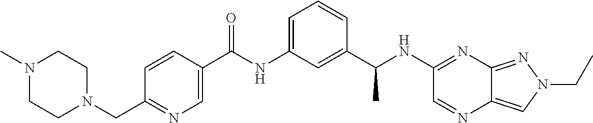 [SMILES: CCn1cc2ncc(N[C@@H](C)c3cccc(NC(=O)c4ccc(CN5CCN(C)CC5)nc4)c3)nc2n1] | (S)-N-(3-(1-((2-ethyl-2H- pyrazolo[3,4-b]pyrazin-6-yl) amino)ethyl)phenyl)-6-((4- methylpiperazin-1-yl)methyl) nicotinamide |
| 388 | 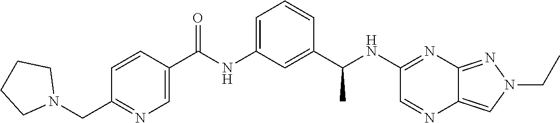 [SMILES: CCn1cc2ncc(N[C@@H](C)c3cccc(NC(=O)c4ccc(CN5CCCC5)nc4)c3)nc2n1] | (S)-N-(3-(1-((2-ethyl-2H- pyrazolo[3,4-b]pyrazin-6-yl) amino)ethyl)phenyl)-6- (pyrrolidin-1-ylmethyl) nicotinamide |
| 389 | 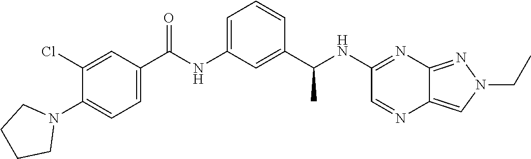 [SMILES: CCn1cc2ncc(N[C@@H](C)c3cccc(NC(=O)c4ccc(N5CCCC5)c(Cl)c4)c3)nc2n1] | (S)-3-chloro-N-(3-(1-((2-ethyl- 2H-pyrazolo[3,4-b]pyrazin-6-yl) amino)ethyl)phenyl)-4- (pyrrolidin-1-yl)benzamide |
| 390 | 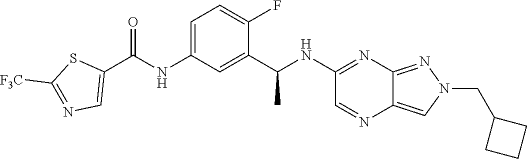 [SMILES: C[C@H](Nc1cnc2cn(CC3CCC3)nc2n1)c1cc(NC(=O)c2cnc(C(F)(F)F)s2)ccc1F] | (S)-N-(3-(1-((2- (cyclobutylmethyl)-2H- pyrazolo[3,4-b]pyrazin-6-yl) amino)ethyl)-4-fluorophenyl)-2- (trifluoromethyl)thiazole-5- carboxamide |
| 391 | 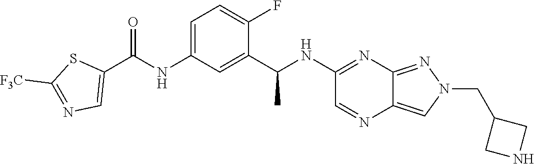 [SMILES: C[C@H](Nc1cnc2cn(CC3CNC3)nc2n1)c1cc(NC(=O)c2cnc(C(F)(F)F)s2)ccc1F] | (S)-N-(3-(1-((2-(azetidin-3- ylmethyl)-2H-pyrazolo[3,4-b] pyrazin-6-yl)amino)ethyl)-4- fluorophenyl)-2- (trifluoromethyl)thiazole-5- carboxamide |
| 392 | 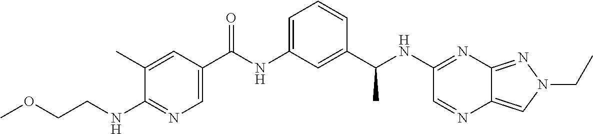 [SMILES: CCn1cc2ncc(N[C@@H](C)c3cccc(NC(=O)c4cnc(NCCOC)c(C)c4)c3)nc2n1] | (S)-N-(3-(1-((2-ethyl-2H- pyrazolo[3,4-b]pyrazin-6-yl) amino)ethyl)phenyl)-6-((2- methoxyethyl)amino)-5- methylnicotinamide |
| 393 | 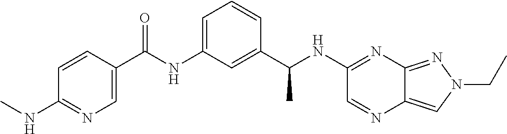 [SMILES: CCn1cc2ncc(N[C@@H](C)c3cccc(NC(=O)c4ccc(NC)nc4)c3)nc2n1] | (S)-N-(3-(1-((2-ethyl-2H- pyrazolo[3,4-b]pyrazin-6-yl) amino)ethyl)phenyl)-6- (methylamino)nicotinamide |
| 394 | 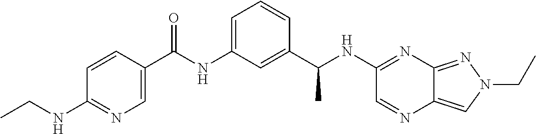 [SMILES: CCNc1ccc(C(=O)Nc2cccc([C@H](C)Nc3cnc4cn(CC)nc4n3)c2)cn1] | (S)-N-(3-(1-((2-ethyl-2H- pyrazolo[3,4-b]pyrazin-6-yl) amino)ethyl)phenyl)-6- (ethylamino)nicotinamide |
| 395 | 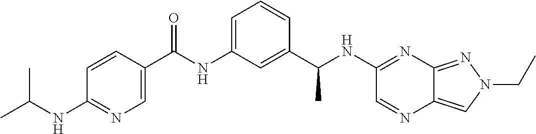 [SMILES: CCn1cc2ncc(N[C@@H](C)c3cccc(NC(=O)c4ccc(NC(C)C)nc4)c3)nc2n1] | (S)-N-(3-(1-((2-ethyl-2H- pyrazolo[3,4-b]pyrazin-6-yl) amino)ethyl)phenyl)-6- (isopropylamino)nicotinamide |
| 396 | 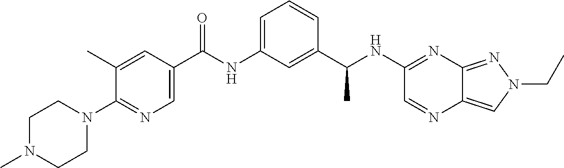 [SMILES: CCn1cc2ncc(N[C@@H](C)c3cccc(NC(=O)c4cnc(N5CCN(C)CC5)c(C)c4)c3)nc2n1] | (S)-N-(3-(1-((2-ethyl-2H- pyrazolo[3,4-b]pyrazin-6-yl) amino)ethyl)phenyl)-5-methyl- 6-(4-methylpiperazin-1-yl) nicotinamide |
| 397 | 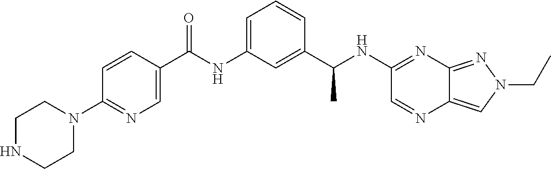 [SMILES: CCn1cc2ncc(N[C@@H](C)c3cccc(NC(=O)c4ccc(N5CCNCC5)nc4)c3)nc2n1] | (S)-N-(3-(1-((2-ethyl-2H- pyrazolo[3,4-b]pyrazin-6-yl) amino)ethyl)phenyl)-6- (piperazin-1-yl)nicotinamide |
| 398 | 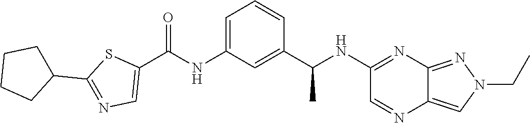 [SMILES: CCn1cc2ncc(N[C@@H](C)c3cccc(NC(=O)c4cnc(C5CCCC5)s4)c3)nc2n1] | (S)-2-cyclopentyl-N-(3-(1-((2- ethyl-2H-pyrazolo[3,4-b] pyrazin-6-yl)amino)ethyl) phenyl)thiazole-5-carboxamide |
| 399 | 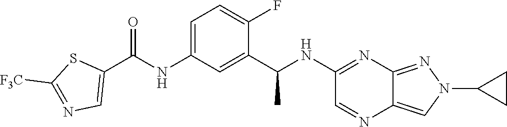 [SMILES: C[C@H](Nc1cnc2cn(C3CC3)nc2n1)c1cc(NC(=O)c2cnc(C(F)(F)F)s2)ccc1F] | (S)-N-(3-(1-((2-cyclopropyl-2H- pyrazolo[3,4-b]pyrazin-6-yl) amino)ethyl)-4-fluorophenyl)-2- (trifluoromethyl)thiazole-5- carboxamide |
| 400 | 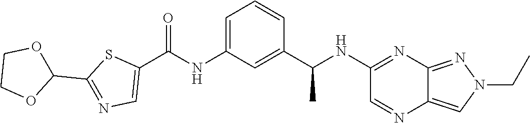 [SMILES: CCn1cc2ncc(N[C@@H](C)c3cccc(NC(=O)c4cnc(C5OCCO5)s4)c3)nc2n1] | (S)-2-(1,3-dioxolan-2-yl)-N-(3- (1-((2-ethyl-2H-pyrazolo[3,4-b] pyrazin-6-yl)amino)ethyl) phenyl)thiazole-5-carboxamide |
| 401 | 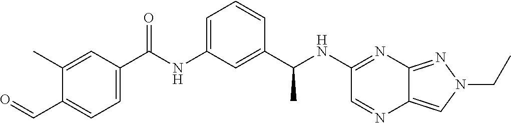 [SMILES: CCn1cc2ncc(N[C@@H](C)c3cccc(NC(=O)c4ccc(C=O)c(C)c4)c3)nc2n1] | (S)-N-(3-(1-((2-ethyl-2H- pyrazolo[3,4-b]pyrazin-6-yl) amino)ethyl)phenyl)-4-formyl- 3-methylbenzamide |
| 402 | 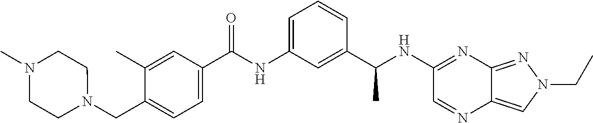 [SMILES: CCn1cc2ncc(N[C@@H](C)c3cccc(NC(=O)c4ccc(CN5CCN(C)CC5)c(C)c4)c3)nc2n1] | (S)-N-(3-(1-((2-ethyl-2H- pyrazolo[3,4-b]pyrazin-6-yl) amino)ethyl)phenyl)-3-methyl- 4-((4-methylpiperazin-1-yl) methyl)benzamide |
| 403 | 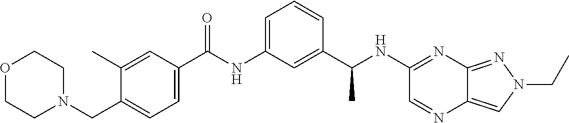 [SMILES: CCn1cc2ncc(N[C@@H](C)c3cccc(NC(=O)c4ccc(CN5CCOCC5)c(C)c4)c3)nc2n1] | (S)-N-(3-(1-((2-ethyl-2H- pyrazolo[3,4-b]pyrazin-6-yl) amino)ethyl)phenyl)-3-methyl- 4-(morpholinomethyl) benzamide |
| 404 | 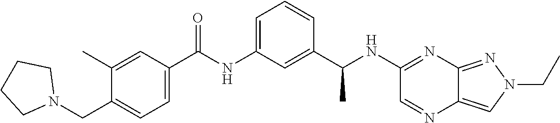 [SMILES: CCn1cc2ncc(N[C@@H](C)c3cccc(NC(=O)c4ccc(CN5CCCC5)c(C)c4)c3)nc2n1] | (S)-N-(3-(1-((2-ethyl-2H- pyrazolo[3,4-b]pyrazin-6-yl) amino)ethyl)phenyl)-3-methyl- 4-(pyrrolidin-1-ylmethyl) benzamide |
| 405 | 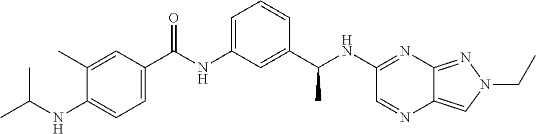 [SMILES: CCn1cc2ncc(N[C@@H](C)c3cccc(NC(=O)c4ccc(NC(C)C)c(C)c4)c3)nc2n1] | (S)-N-(3-(1-((2-ethyl-2H- pyrazolo[3,4-b]pyrazin-6-yl) amino)ethyl)phenyl)-4- (isopropylamino)-3- methylbenzamide |
| 406 | 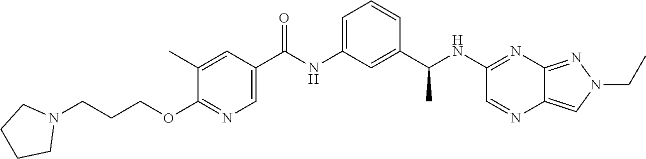 [SMILES: CCn1cc2ncc(N[C@@H](C)c3cccc(NC(=O)c4cnc(OCCCN5CCCC5)c(C)c4)c3)nc2n1] | (S)-N-(3-(1-((2-ethyl-2H- pyrazolo[3,4-b]pyrazin-6-yl) amino)ethyl)phenyl)-5-methyl- 6-(3-(pyrrolidin-1-yl) propoxy)nicotinamide |
| 407 | 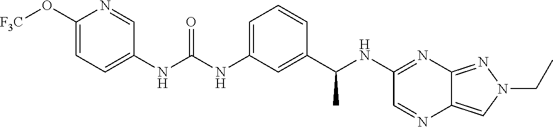 [SMILES: CCn1cc2ncc(N[C@@H](C)c3cccc(NC(=O)Nc4ccc(OC(F)(F)F)nc4)c3)nc2n1] | (S)-1-(3-(1-((2-ethyl-2H- pyrazolo[3,4-b]pyrazin-6-yl) amino)ethyl)phenyl)-3-(6- (trifluoromethoxy)pyridin-3-yl) urea |
| 408 | 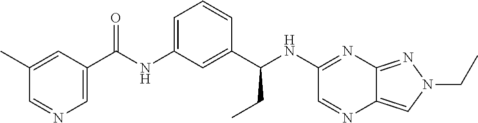 [SMILES: CC[C@H](Nc1cnc2cn(CC)nc2n1)c1cccc(NC(=O)c2cncc(C)c2)c1] | (S)-N-(3-(1-((2-ethyl-2H- pyrazolo[3,4-b]pyrazin-6-yl) amino)propyl)phenyl)-5- methylnicotinamide |
| 409 | 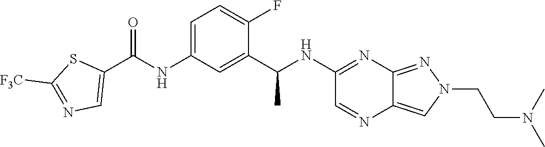 [SMILES: C[C@H](Nc1cnc2cn(CCN(C)C)nc2n1)c1cc(NC(=O)c2cnc(C(F)(F)F)s2)ccc1F] | (S)-N-(3-(1-((2-(2- (dimethylamino)ethyl)-2H- pyrazolo[3,4-b]pyrazin-6-yl) amino)ethyl)-4-fluorophenyl)-2- (trifluoromethyl)thiazole-5- carboxamide |
| 410 | 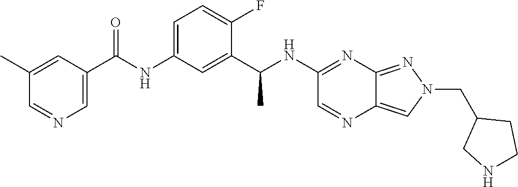 [SMILES: Cc1cncc(C(=O)Nc2ccc(F)c([C@H](C)Nc3cnc4cn(CC5CCNC5)nc4n3)c2)c1] | N-(4-fluoro-3-((1S)-1-((2- (pyrrolidin-3-ylmethyl)-2H- pyrazolo[3,4-b]pyrazin-6-yl) amino)ethyl)phenyl)-5- methylnicotinamide |
| 411 | 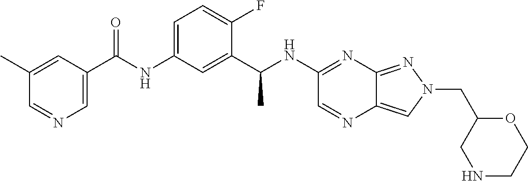 [SMILES: Cc1cncc(C(=O)Nc2ccc(F)c([C@H](C)Nc3cnc4cn(CC5CNCCO5)nc4n3)c2)c1] | N-(4-fluoro-3-((1S)-1-((2- (morpholin-2-ylmethyl)-2H- pyrazolo[3,4-b]pyrazin-6-yl) amino)ethyl)phenyl)-5- methylnicotinamide |
| 412 | 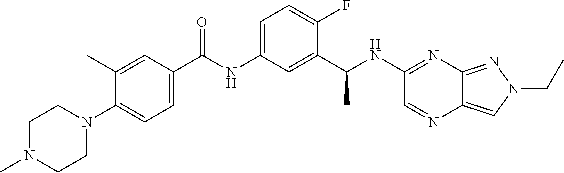 [SMILES: CCn1cc2ncc(N[C@@H](C)c3cc(NC(=O)c4ccc(N5CCN(C)CC5)c(C)c4)ccc3F)nc2n1] | (S)-N-(3-(1-((2-ethyl-2H- pyrazolo[3,4-b]pyrazin-6-yl) amino)ethyl)-4-fluorophenyl)-3- methyl-4-(4-methylpiperazin-1- yl)benzamide |
| 413 | 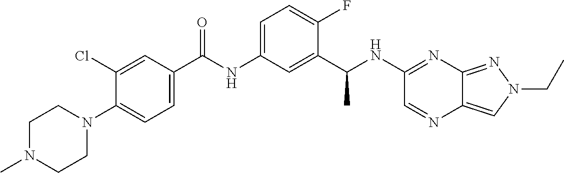 [SMILES: CCn1cc2ncc(N[C@@H](C)c3cc(NC(=O)c4ccc(N5CCN(C)CC5)c(Cl)c4)ccc3F)nc2n1] | (S)-3-chloro-N-(3-(1-((2-ethyl- 2H-pyrazolo[3,4-b]pyrazin-6-yl) amino)ethyl)-4-fluorophenyl)-4- (4-methylpiperazin-1-yl) benzamide |
| 414 | 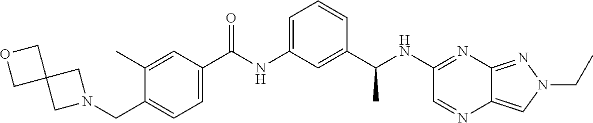 [SMILES: CCn1cc2ncc(N[C@@H](C)c3cccc(NC(=O)c4ccc(CN5CC6(COC6)C5)c(C)c4)c3)nc2n1] | (S)-4-((2-oxa-6- azaspiro[3.3]heptan-6-yl) methyl)-N-(3-(1-((2-ethyl-2H- pyrazolo[3,4-b]pyrazin-6-yl) amino)ethyl)phenyl)-3- methylbenzamide |
| 415 | 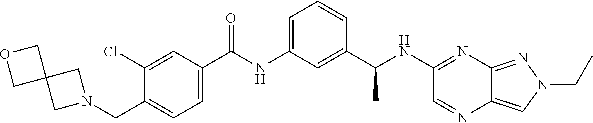 [SMILES: CCn1cc2ncc(N[C@@H](C)c3cccc(NC(=O)c4ccc(CN5CC6(COC6)C5)c(Cl)c4)c3)nc2n1] | (S)-4-((2-oxa-6- azaspiro[3.3]heptan-6-yl) methyl)-3-chloro-N-(3-(1-((2- ethyl-2H-pyrazolo[3,4-b] pyrazin-6-yl)amino)ethyl) phenyl)benzamide |
| 416 | 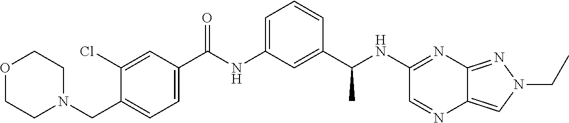 [SMILES: CCn1cc2ncc(N[C@@H](C)c3cccc(NC(=O)c4ccc(CN5CCOCC5)c(Cl)c4)c3)nc2n1] | (S)-3-chloro-N-(3-(1-((2-ethyl- 2H-pyrazolo[3,4-b]pyrazin-6-yl) amino)ethyl)phenyl)-4- (morpholinomethyl)benzamide |
| 417 | 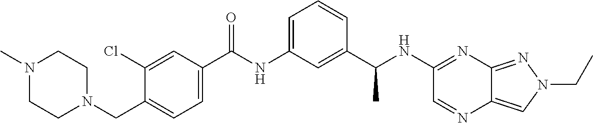 [SMILES: CCn1cc2ncc(N[C@@H](C)c3cccc(NC(=O)c4ccc(CN5CCN(C)CC5)c(Cl)c4)c3)nc2n1] | (S)-3-chloro-N-(3-(1-((2-ethyl- 2H-pyrazolo[3,4-b]pyrazin-6-yl) amino)ethyl)phenyl)-4-((4- methylpiperazin-1-yl)methyl) benzamide |
| 418 | 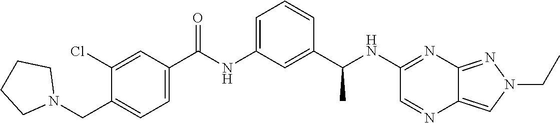 [SMILES: CCn1cc2ncc(N[C@@H](C)c3cccc(NC(=O)c4ccc(CN5CCCC5)c(Cl)c4)c3)nc2n1] | (S)-3-chloro-N-(3-(1-((2-ethyl- 2H-pyrazolo[3,4-b]pyrazin-6-yl) amino)ethyl)phenyl)-4- (pyrrolidin-1-ylmethyl) benzamide |
| 419 | 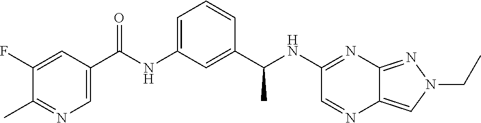 [SMILES: CCn1cc2ncc(N[C@@H](C)c3cccc(NC(=O)c4cnc(C)c(F)c4)c3)nc2n1] | (S)-N-(3-(1-((2-ethyl-2H- pyrazolo[3,4-b]pyrazin-6-yl) amino)ethyl)phenyl)-5-fluoro-6- methylnicotinamide |
| 420 | 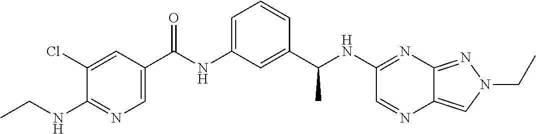 [SMILES: CCNc1ncc(C(=O)Nc2cccc([C@H](C)Nc3cnc4cn(CC)nc4n3)c2)cc1Cl] | (S)-5-chloro-N-(3-(1-((2-ethyl- 2H-pyrazolo[3,4-b]pyrazin-6-yl) amino)ethyl)phenyl)-6- (ethylamino)nicotinamide |
| 421 | 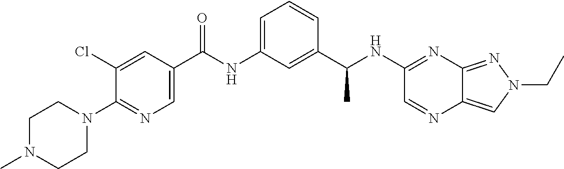 [SMILES: CCn1cc2ncc(N[C@@H](C)c3cccc(NC(=O)c4cnc(N5CCN(C)CC5)c(Cl)c4)c3)nc2n1] | (S)-5-chloro-N-(3-(1-((2-ethyl- 2H-pyrazolo[3,4-b]pyrazin-6-yl) amino)ethyl)phenyl)-6-(4- methylpiperazin-1-yl) nicotinamide |
| 422 | 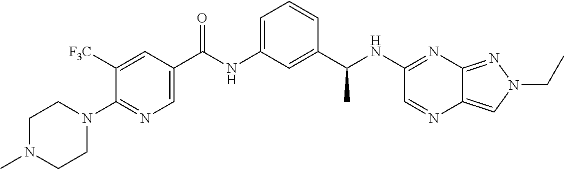 [SMILES: CCn1cc2ncc(N[C@@H](C)c3cccc(NC(=O)c4cnc(N5CCN(C)CC5)c(C(F)(F)F)c4)c3)nc2n1] | (S)-N-(3-(1-((2-ethyl-2H- pyrazolo[3,4-b]pyrazin-6-yl) amino)ethyl)phenyl)-6-(4- methylpiperazin-1-yl)-5- (trifluoromethyl)nicotinamide |
| 423 | 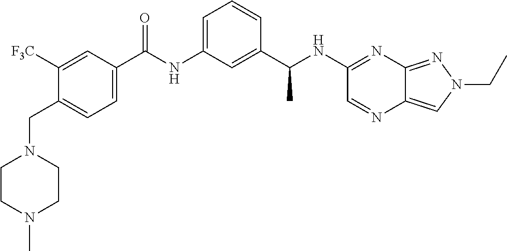 [SMILES: CCn1cc2ncc(N[C@@H](C)c3cccc(NC(=O)c4ccc(CN5CCN(C)CC5)c(C(F)(F)F)c4)c3)nc2n1] | (S)-N-(3-(1-((2-ethyl-2H- pyrazolo[3,4-b]pyrazin-6-yl) amino)ethyl)phenyl)-4-((4- methylpiperazin-1-yl)methyl)-3- (trifluoromethyl)benzamide |
| 424 | 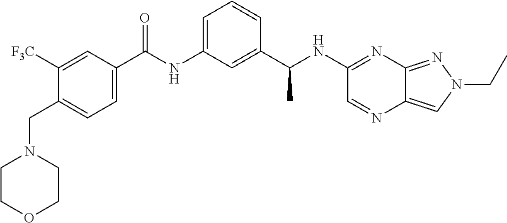 [SMILES: CCn1cc2ncc(N[C@@H](C)c3cccc(NC(=O)c4ccc(CN5CCOCC5)c(C(F)(F)F)c4)c3)nc2n1] | (S)-N-(3-(1-((2-ethyl-2H- pyrazolo[3,4-b]pyrazin-6-yl) amino)ethyl)phenyl)-4- (morpholinomethyl)-3- (trifluoromethyl)benzamide |
| 425 | 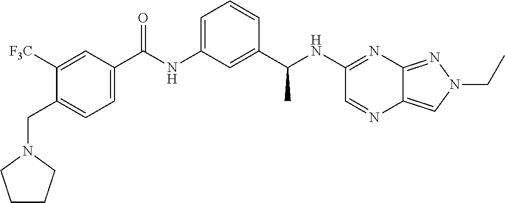 [SMILES: CCn1cc2ncc(N[C@@H](C)c3cccc(NC(=O)c4ccc(CN5CCCC5)c(C(F)(F)F)c4)c3)nc2n1] | (S)-N-(3-(1-((2-ethyl-2H- pyrazolo[3,4-b]pyrazin-6-yl) amino)ethyl)phenyl)-4- (pyrrolidin-1-ylmethyl)-3- (trifluoromethyl)benzamide |
| 426 | 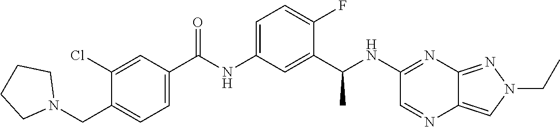 [SMILES: CCn1cc2ncc(N[C@@H](C)c3cc(NC(=O)c4ccc(CN5CCCC5)c(Cl)c4)ccc3F)nc2n1] | (S)-3-chloro-N-(3-(1-((2-ethyl- 2H-pyrazolo[3,4-b]pyrazin-6-yl) amino)ethyl)-4-fluorophenyl)-4- (pyrrolidin-1-ylmethyl) benzamide |
| 427 | 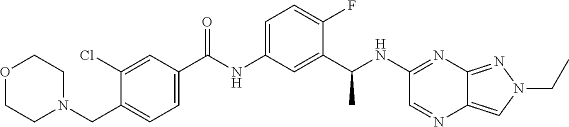 [SMILES: CCn1cc2ncc(N[C@@H](C)c3cc(NC(=O)c4ccc(CN5CCOCC5)c(Cl)c4)ccc3F)nc2n1] | (S)-3-chloro-N-(3-(1-((2-ethyl- 2H-pyrazolo[3,4-b]pyrazin-6-yl) amino)ethyl)-4-fluorophenyl)-4- (morpholinomethyl)benzamide |
| 428 | 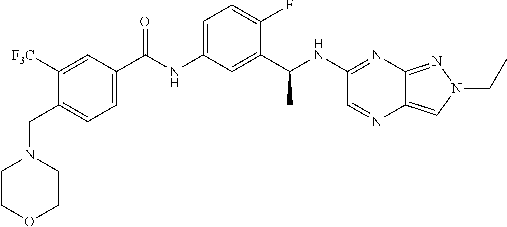 [SMILES: CCn1cc2ncc(N[C@@H](C)c3cc(NC(=O)c4ccc(CN5CCOCC5)c(C(F)(F)F)c4)ccc3F)nc2n1] | (S)-N-(3-(1-((2-ethyl-2H- pyrazolo[3,4-b]pyrazin-6-yl) amino)ethyl)-4-fluorophenyl)-4- (morpholinomethyl)-3- (trifluoromethyl)benzamide |
| 429 | 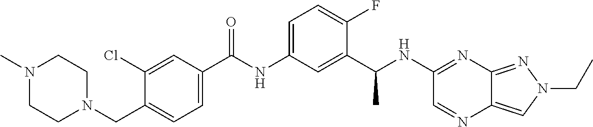 [SMILES: CCn1cc2ncc(N[C@@H](C)c3cc(NC(=O)c4ccc(CN5CCN(C)CC5)c(Cl)c4)ccc3F)nc2n1] | (S)-3-chloro-N-(3-(1-((2-ethyl- 2H-pyrazolo[3,4-b]pyrazin-6-yl) amino)ethyl)-4-fluorophenyl)-4- ((4-methylpiperazin-1-yl) methyl)benzamide |
| 430 | 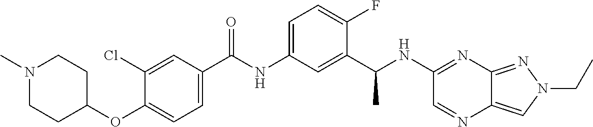 [SMILES: CCn1cc2ncc(N[C@@H](C)c3cc(NC(=O)c4ccc(OC5CCN(C)CC5)c(Cl)c4)ccc3F)nc2n1] | (S)-3-chloro-N-(3-(1-((2-ethyl- 2H-pyrazolo[3,4-b]pyrazin-6-yl) amino)ethyl)-4-fluorophenyl)-4- ((1-methylpiperidin-4-yl) oxy)benzamide |
| 431 | 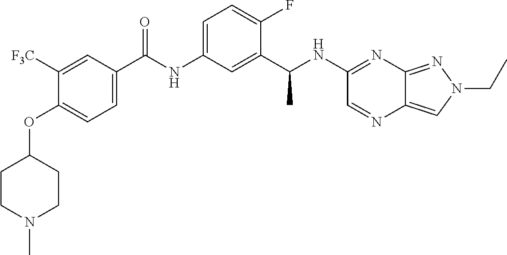 [SMILES: CCn1cc2ncc(N[C@@H](C)c3cc(NC(=O)c4ccc(OC5CCN(C)CC5)c(C(F)(F)F)c4)ccc3F)nc2n1] | (S)-N-(3-(1-((2-ethyl-2H- pyrazolo[3,4-b]pyrazin-6-yl) amino)ethyl)-4-fluorophenyl)-4- ((1-methylpiperidin-4-yl)oxy)- 3-(trifluoromethyl)benzamide |
| 432 | 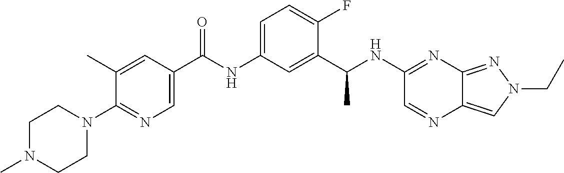 [SMILES: CCn1cc2ncc(N[C@@H](C)c3cc(NC(=O)c4cnc(N5CCN(C)CC5)c(C)c4)ccc3F)nc2n1] | (S)-N-(3-(1-((2-ethyl-2H- pyrazolo[3,4-b]pyrazin-6-yl) amino)ethyl)-4-fluorophenyl)-5- methyl-6-(4-methylpiperazin-1- yl)nicotinamide |
| 433 | 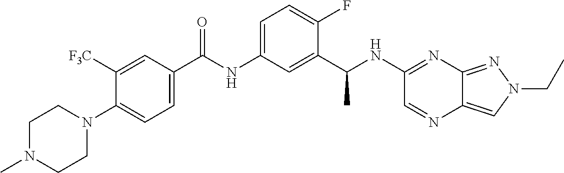 [SMILES: CCn1cc2ncc(N[C@@H](C)c3cc(NC(=O)c4ccc(N5CCN(C)CC5)c(C(F)(F)F)c4)ccc3F)nc2n1] | (S)-N-(3-(1-((2-ethyl-2H- pyrazolo[3,4-b]pyrazin-6-yl) amino)ethyl)-4-fluorophenyl)-4- (4-methylpiperazin-1-yl)-3- (trifluoromethyl)benzamide |
| 434 | 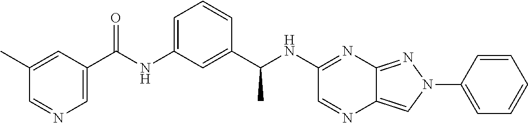 [SMILES: Cc1cncc(C(=O)Nc2cccc([C@H](C)Nc3cnc4cn(-c5ccccc5)nc4n3)c2)c1] | (S)-5-methyl-N-(3-(1-((2- phenyl-2H-pyrazolo[3,4-b] pyrazin-6-yl)amino)ethyl) phenyl)nicotinamide |
| 435 | 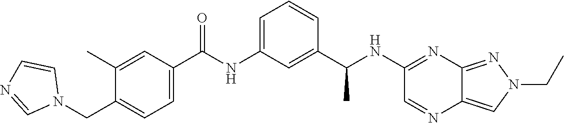 [SMILES: CCn1cc2ncc(N[C@@H](C)c3cccc(NC(=O)c4ccc(Cn5ccnc5)c(C)c4)c3)nc2n1] | (S)-4-((1H-imidazol-1-yl) methyl)-N-(3-(1-((2-ethyl-2H- pyrazolo[3,4-b]pyrazin-6-yl) amino)ethyl)phenyl)-3- methylbenzamide |
| 436 | 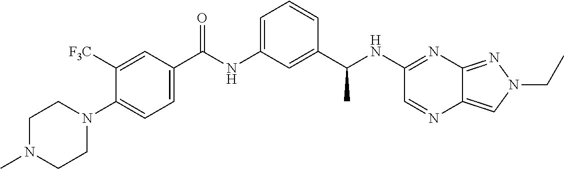 [SMILES: CCn1cc2ncc(N[C@@H](C)c3cccc(NC(=O)c4ccc(N5CCN(C)CC5)c(C(F)(F)F)c4)c3)nc2n1] | (S)-N-(3-(1-((2-ethyl-2H- pyrazolo[3,4-b]pyrazin-6-yl) amino)ethyl)phenyl)-4-(4- methylpiperazin-1-yl)-3- (trifluoromethyl)benzamide |
| 437 | 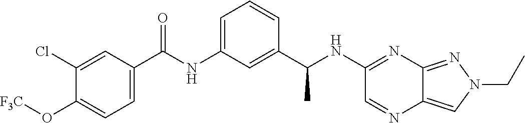 [SMILES: CCn1cc2ncc(N[C@@H](C)c3cccc(NC(=O)c4ccc(OC(F)(F)F)c(Cl)c4)c3)nc2n1] | (S)-3-chloro-N-(3-(1-((2-ethyl- 2H-pyrazolo[3,4-b]pyrazin-6-yl) amino)ethyl)phenyl)-4- (trifluoromethoxy)benzamide |
| 438 | 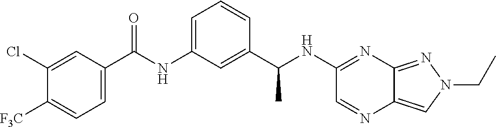 [SMILES: CCn1cc2ncc(N[C@@H](C)c3cccc(NC(=O)c4ccc(C(F)(F)F)c(Cl)c4)c3)nc2n1] | (S)-3-chloro-N-(3-(1-((2-ethyl- 2H-pyrazolo[3,4-b]pyrazin-6-yl) amino)ethyl)phenyl)-4- (trifluoromethyl)benzamide |
| 439 | 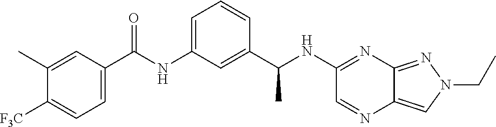 [SMILES: CCn1cc2ncc(N[C@@H](C)c3cccc(NC(=O)c4ccc(C(F)(F)F)c(C)c4)c3)nc2n1] | (S)-N-(3-(1-((2-ethyl-2H- pyrazolo[3,4-b]pyrazin-6-yl) amino)ethyl)phenyl)-3-methyl- 4-(trifluoromethyl)benzamide |
| 440 | 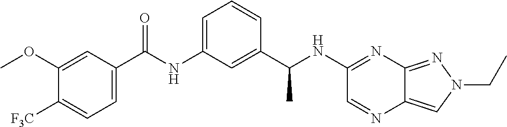 [SMILES: CCn1cc2ncc(N[C@@H](C)c3cccc(NC(=O)c4ccc(C(F)(F)F)c(OC)c4)c3)nc2n1] | (S)-N-(3-(1-((2-ethyl-2H- pyrazolo[3,4-b]pyrazin-6-yl) amino)ethyl)phenyl)-3- methoxy-4-(trifluoromethyl) benzamide |
| 441 | 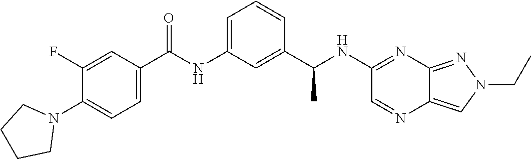 [SMILES: CCn1cc2ncc(N[C@@H](C)c3cccc(NC(=O)c4ccc(N5CCCC5)c(F)c4)c3)nc2n1] | (S)-N-(3-(1-((2-ethyl-2H- pyrazolo[3,4-b]pyrazin-6-yl) amino)ethyl)phenyl)-3-fluoro-4- (pyrrolidin-1-yl)benzamide |
| 442 | 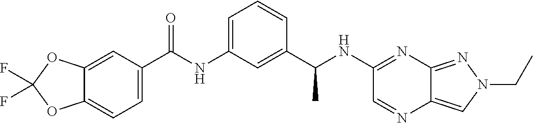 [SMILES: CCn1cc2ncc(N[C@@H](C)c3cccc(NC(=O)c4ccc5c(c4)OC(F)(F)O5)c3)nc2n1] | (S)-N-(3-(1-((2-ethyl-2H- pyrazolo[3,4-b]pyrazin-6-yl) amino)ethyl)phenyl)-2,2- difluorobenzo[d][1,3]dioxole-5- carboxamide |
| 443 | 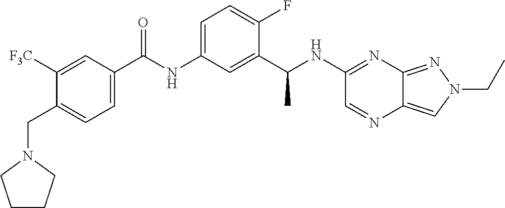 [SMILES: CCn1cc2ncc(N[C@@H](C)c3cc(NC(=O)c4ccc(CN5CCCC5)c(C(F)(F)F)c4)ccc3F)nc2n1] | (S)-N-(3-(1-((2-ethyl-2H- pyrazolo[3,4-b]pyrazin-6-yl) amino)ethyl)-4-fluorophenyl)-4- (pyrrolidin-1-ylmethyl)-3- (trifluoromethyl)benzamide |
| 444 | 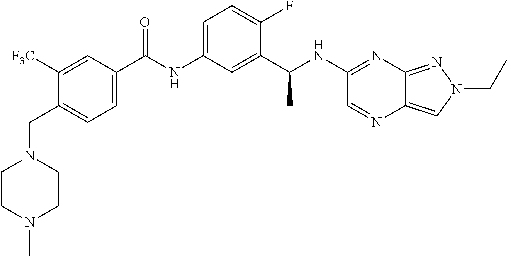 [SMILES: CCn1cc2ncc(N[C@@H](C)c3cc(NC(=O)c4ccc(CN5CCN(C)CC5)c(C(F)(F)F)c4)ccc3F)nc2n1] | (S)-N-(3-(1-((2-ethyl-2H- pyrazolo[3,4-b]pyrazin-6-yl) amino)ethyl)-4-fluorophenyl)-4- ((4-methylpiperazin-1-yl) methyl)-3-(trifluoromethyl) benzamide |
| 445 | 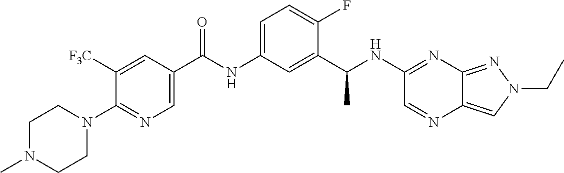 [SMILES: CCn1cc2ncc(N[C@@H](C)c3cc(NC(=O)c4cnc(N5CCN(C)CC5)c(C(F)(F)F)c4)ccc3F)nc2n1] | (S)-N-(3-(1-((2-ethyl-2H- pyrazolo[3,4-b]pyrazin-6-yl) amino)ethyl)-4-fluorophenyl)-6- (4-methylpiperazin-1-yl)-5- (trifluoromethyl)nicotinamide |
| 446 | 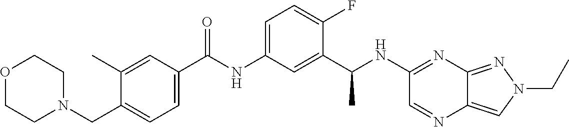 [SMILES: CCn1cc2ncc(N[C@@H](C)c3cc(NC(=O)c4ccc(CN5CCOCC5)c(C)c4)ccc3F)nc2n1] | (S)-N-(3-(1-((2-ethyl-2H- pyrazolo[3,4-b]pyrazin-6-yl) amino)ethyl)-4-fluorophenyl)-3- methyl-4-(morpholinomethyl) benzamide |
| 447 | 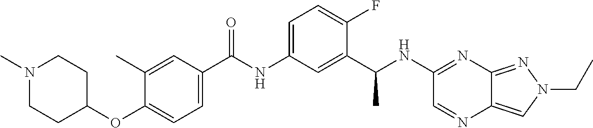 [SMILES: CCn1cc2ncc(N[C@@H](C)c3cc(NC(=O)c4ccc(OC5CCN(C)CC5)c(C)c4)ccc3F)nc2n1] | (S)-N-(3-(1-((2-ethyl-2H- pyrazolo[3,4-b]pyrazin-6-yl) amino)ethyl)-4-fluorophenyl)-3- methyl-4-((1-methylpiperidin-4- yl)oxy)benzamide |
| 448 | 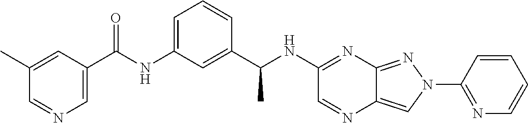 [SMILES: Cc1cncc(C(=O)Nc2cccc([C@H](C)Nc3cnc4cn(-c5ccccn5)nc4n3)c2)c1] | (S)-5-methyl-N-(3-(1-((2- (pyridin-2-yl)-2H-pyrazolo[3,4- b]pyrazin-6-yl)amino)ethyl) phenyl)nicotinamide |
| 449 | 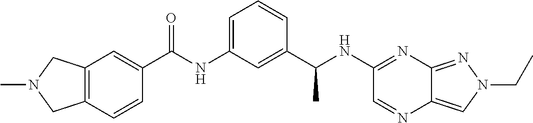 [SMILES: CCn1cc2ncc(N[C@@H](C)c3cccc(NC(=O)c4ccc5c(c4)CN(C)C5)c3)nc2n1] | (S)-N-(3-(1-((2-ethyl-2H- pyrazolo[3,4-b]pyrazin-6-yl) amino)ethyl)phenyl)-2- methylisoindoline-5- carboxamide |
| 450 | 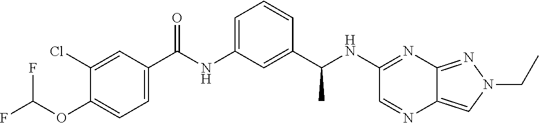 [SMILES: CCn1cc2ncc(N[C@@H](C)c3cccc(NC(=O)c4ccc(OC(F)F)c(Cl)c4)c3)nc2n1] | (S)-3-chloro-4- (difluoromethoxy)-N-(3-(1-((2- ethyl-2H-pyrazolo[3,4-b] pyrazin-6-yl)amino)ethyl) phenyl)benzamide |
| 451 | 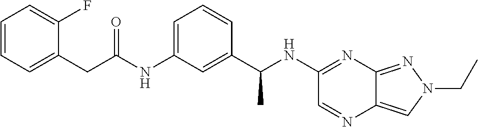 [SMILES: CCn1cc2ncc(N[C@@H](C)c3cccc(NC(=O)Cc4ccccc4F)c3)nc2n1] | (S)-N-(3-(1-((2-ethyl-2H- pyrazolo[3,4-b]pyrazin-6-yl) amino)ethyl)phenyl)-2-(2- fluorophenyl)acetamide |
| 452 | 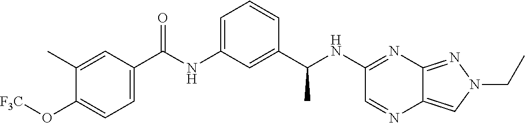 [SMILES: CCn1cc2ncc(N[C@@H](C)c3cccc(NC(=O)c4ccc(OC(F)(F)F)c(C)c4)c3)nc2n1] | (S)-N-(3-(1-((2-ethyl-2H- pyrazolo[3,4-b]pyrazin-6-yl) amino)ethyl)phenyl)-3-methyl- 4-(trifluoromethoxy)benzamide |
| 453 | 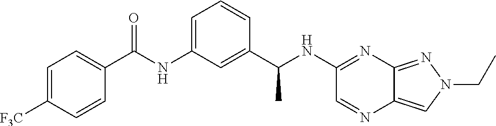 [SMILES: CCn1cc2ncc(N[C@@H](C)c3cccc(NC(=O)c4ccc(C(F)(F)F)cc4)c3)nc2n1] | (S)-N-(3-(1-((2-ethyl-2H- pyrazolo[3,4-b]pyrazin-6-yl) amino)ethyl)phenyl)-4- (trifluoromethyl)benzamide |
| 454 | 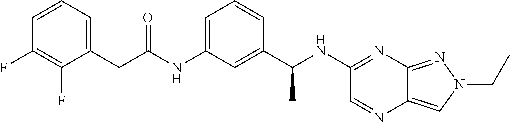 [SMILES: CCn1cc2ncc(N[C@@H](C)c3cccc(NC(=O)Cc4cccc(F)c4F)c3)nc2n1] | (S)-2-(2,3-difluorophenyl)-N-(3- (1-((2-ethyl-2H-pyrazolo[3,4-b] pyrazin-6-yl)amino)ethyl) phenyl)acetamide |
| 455 | 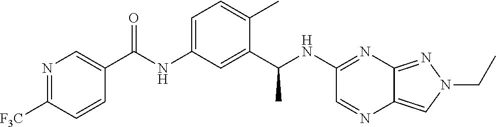 [SMILES: CCn1cc2ncc(N[C@@H](C)c3cc(NC(=O)c4ccc(C(F)(F)F)nc4)ccc3C)nc2n1] | (S)-N-(3-(1-((2-ethyl-2H- pyrazolo[3,4-b]pyrazin-6-yl) amino)ethyl)-4-methylphenyl)- 6-(trifluoromethyl)nicotinamide |
| 456 | 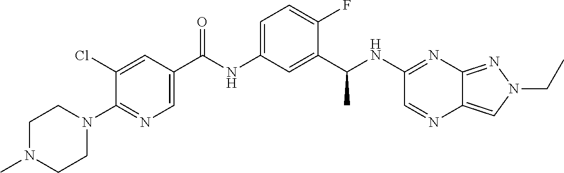 [SMILES: CCn1cc2ncc(N[C@@H](C)c3cc(NC(=O)c4cnc(N5CCN(C)CC5)c(Cl)c4)ccc3F)nc2n1] | (S)-5-chloro-N-(3-(1-((2-ethyl- 2H-pyrazolo[3,4-b]pyrazin-6-yl) amino)ethyl)-4-fluorophenyl)-6- (4-methylpiperazin-1-yl) nicotinamide |
| 457 | 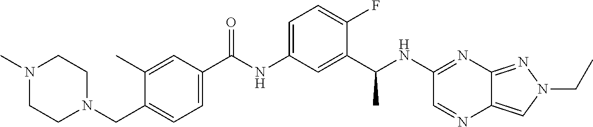 [SMILES: CCn1cc2ncc(N[C@@H](C)c3cc(NC(=O)c4ccc(CN5CCN(C)CC5)c(C)c4)ccc3F)nc2n1] | (S)-N-(3-(1-((2-ethyl-2H- pvrazolo[3,4-b]pyrazin-6-yl) amino)ethyl)-4-fluorophenyl)-3 methyl-4-((4-methylpiperazin-1- yl)methyl)benzamide |
| 458 | 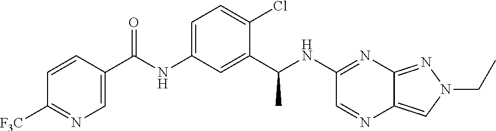 [SMILES: CCn1cc2ncc(N[C@@H](C)c3cc(NC(=O)c4ccc(C(F)(F)F)nc4)ccc3Cl)nc2n1] | (S)-N-(4-chloro-3-(1-((2-ethyl- 2H-pyrazolo[3,4-b]pyrazin-6-yl) amino)ethyl)phenyl)-6- (trifluoromethyl)nicotinamide |
| 459 | 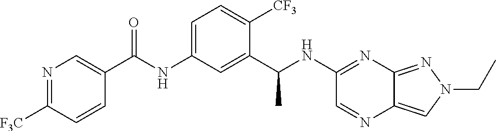 [SMILES: CCn1cc2ncc(N[C@@H](C)c3cc(NC(=O)c4ccc(C(F)(F)F)nc4)ccc3C(F)(F)F)nc2n1] | (S)-N-(3-(1-((2-ethyl-2H- pyrazolo[3,4-b]pyrazin-6-yl) amino)ethyl)-4-(trifluoromethyl) phenyl)-6-(trifluoromethyl) nicotinamide |
| 460 | 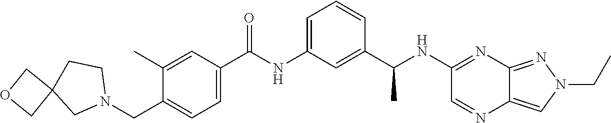 [SMILES: CCn1cc2ncc(N[C@@H](C)c3cccc(NC(=O)c4ccc(CN5CCC6(COC6)C5)c(C)c4)c3)nc2n1] | (S)-4-((2-oxa-6- azaspiro[3.4]octan-6-yl)methyl)- N-(3-(1-((2-ethyl-2H- pyrazolo[3,4-b]pyrazin-6-yl) amino)ethyl)phenyl)-3- methylbenzamide |
| 461 | 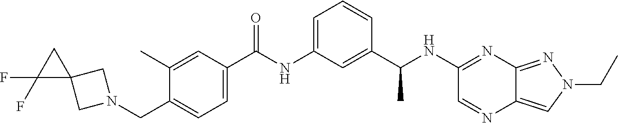 [SMILES: CCn1cc2ncc(N[C@@H](C)c3cccc(NC(=O)c4ccc(CN5CC6(C5)CC6(F)F)c(C)c4)c3)nc2n1] | (S)-4-((1,1-difluoro-5- azaspiro[2.3]hexan-5-yl)methyl)- 1-((2-ethyl-2H- pyrazolo[3,4-b]pyrazin-6-yl) amino)ethyl)phenyl)-3- methylbenzamide |
| 462 | 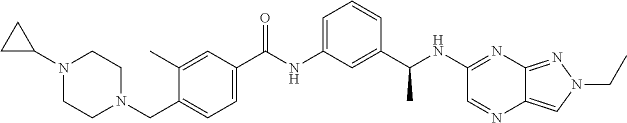 [SMILES: CCn1cc2ncc(N[C@@H](C)c3cccc(NC(=O)c4ccc(CN5CCN(C6CC6)CC5)c(C)c4)c3)nc2n1] | (S)-4-((4-cyclopropylpiperazin- 1-yl)methyl)-N-(3-(1-((2-ethyl- 2H-pyrazolo[3,4-b]pyrazin-6-yl) amino)ethyl)phenyl)-3- methylbenzamide |
| 463 | 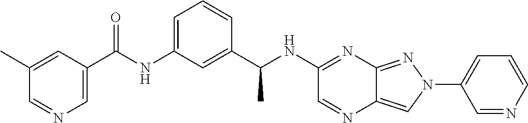 [SMILES: Cc1cncc(C(=O)Nc2cccc([C@H](C)Nc3cnc4cn(-c5cccnc5)nc4n3)c2)c1] | (S)-5-methyl-N-(3-(1-((2- (pyridin-3-yl)-2H-pyrazolo[3,4- b]pyrazin-6-yl)amino)ethyl) phenyl)nicotinamide |
| 464 | 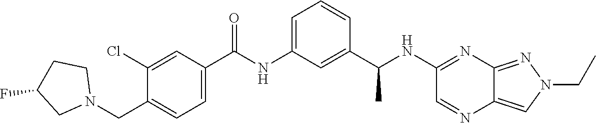 [SMILES: CCn1cc2ncc(N[C@@H](C)c3cccc(NC(=O)c4ccc(CN5CC[C@@H](F)C5)c(Cl)c4)c3)nc2n1] | 3-chloro-N-(3-((S)-1-((2-ethyl- 2H-pyrazolo[3,4-b]pyrazin-6-yl) amino)ethyl)phenyl)-4-(((R)-3- fluoropyrrolidin-1-yl)methyl) benzamide |
| 465 | 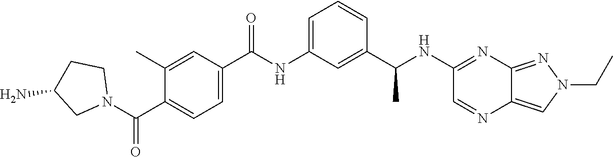 [SMILES: CCn1cc2ncc(N[C@@H](C)c3cccc(NC(=O)c4ccc(C(=O)N5CC[C@@H](N)C5)c(C)c4)c3)nc2n1] | 4-((R)-3-aminopyrrolidine-1- carbonyl)-N-(3-((S)-1-((2-ethyl- 2H-pyrazolo[3,4-b]pyrazin-6-yl) amino)ethyl)phenyl)-3- methylbenzamide |
| 466 | 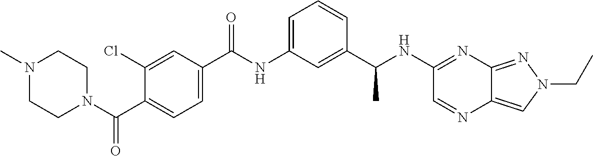 [SMILES: CCn1cc2ncc(N[C@@H](C)c3cccc(NC(=O)c4ccc(C(=O)N5CCN(C)CC5)c(Cl)c4)c3)nc2n1] | (S)-3-chloro-N-(3-(1-((2-ethyl- 2H-pyrazolo[3,4-b]pyrazin-6-yl) amino)ethyl)phenyl)-4-(4- methylpiperazine-1-carbonyl) benzamide |
| 467 | 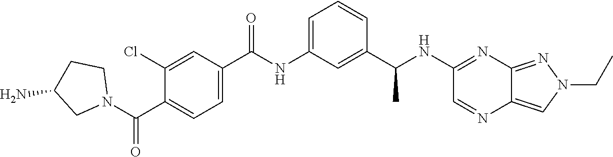 [SMILES: CCn1cc2ncc(N[C@@H](C)c3cccc(NC(=O)c4ccc(C(=O)N5CC[C@@H](N)C5)c(Cl)c4)c3)nc2n1] | 4-((R)-3-aminopyrrolidine-1- carbonyl)-3-chloro-N-(3-((5)-1- ((2-ethyl-2H-pyrazolo[3,4-b] pyrazin-6-yl)amino)ethyl) phenyl)benzamide |
| 468 | 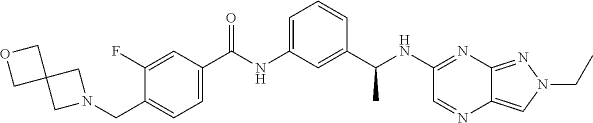 [SMILES: CCn1cc2ncc(N[C@@H](C)c3cccc(NC(=O)c4ccc(CN5CC6(COC6)C5)c(F)c4)c3)nc2n1] | (S)-4-((2-oxa-6- azaspiro[3.3]heptan-6-yl) methyl)-N-(3-(1-((2-ethyl-2H- pyrazolo[3,4-b]pyrazin-6-yl) amino)ethyl)phenyl)-3- fluorobenzamide |
| 469 | 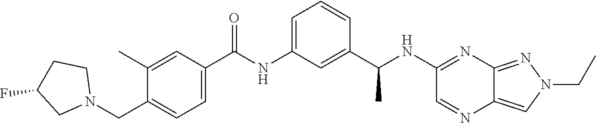 [SMILES: CCn1cc2ncc(N[C@@H](C)c3cccc(NC(=O)c4ccc(CN5CC[C@@H](F)C5)c(C)c4)c3)nc2n1] | N-(3-((S)-1-((2-ethyl-2H- pyrazolo[3,4-b]pyrazin-6-yl) amino)ethyl)phenyl)-4-(((R)-3- fluoropyrrolidin-1-yl)methyl)-3- methylbenzamide |
| 470 | 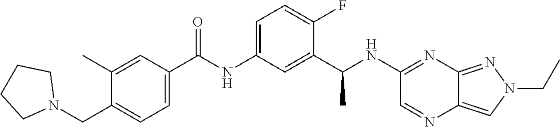 [SMILES: CCn1cc2ncc(N[C@@H](C)c3cc(NC(=O)c4ccc(CN5CCCC5)c(C)c4)ccc3F)nc2n1] | (S)-N-(3-(1-((2-ethyl-2H- pyrazolo[3,4-b]pyrazin-6-yl) amino)ethyl)-4-fluorophenyl)-3- methyl-4-(pyrrolidin-1- ylmethyl)benzamide |
| 471 | 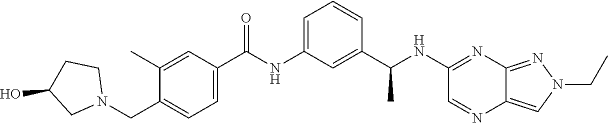 [SMILES: CCn1cc2ncc(N[C@@H](C)c3cccc(NC(=O)c4ccc(CN5CC[C@H](O)C5)c(C)c4)c3)nc2n1] | N-(3-((S)-1-((2-ethyl-2H- pyrazolo[3,4-b]pyrazin-6-yl) amino)ethyl)phenyl)-4-(((S)-3- hydroxypyrrolidin-1-yl)methyl)- 3-methylbenzamide |
| 472 | 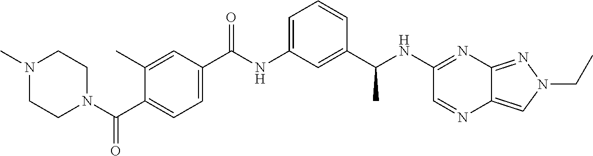 [SMILES: CCn1cc2ncc(N[C@@H](C)c3cccc(NC(=O)c4ccc(C(=O)N5CCN(C)CC5)c(C)c4)c3)nc2n1] | (S)-N-(3-(1-((2-ethyl-2H- pyrazolo[3,4-b]pyrazin-6-yl) amino)ethyl)phenyl)-3-methyl- 4-(4-methylpiperazine-1- carbonyl)benzamide |
| 473 | 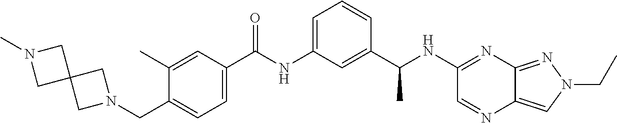 [SMILES: CCn1cc2ncc(N[C@@H](C)c3cccc(NC(=O)c4ccc(CN5CC6(CN(C)C6)C5)c(C)c4)c3)nc2n1] | (S)-N-(3-(1-((2-ethyl-2H- pyrazolo[3,4-b]pyrazin-6-yl) amino)ethyl)phenyl)-3-methyl- 4-((6-methyl-2,6- diazaspiro[3.3]heptan-2-yl) methyl)benzamide |
| 474 | 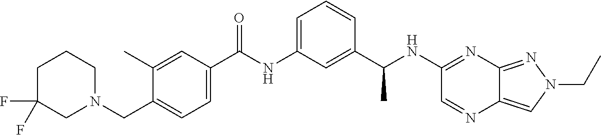 [SMILES: CCn1cc2ncc(N[C@@H](C)c3cccc(NC(=O)c4ccc(CN5CCCC(F)(F)C5)c(C)c4)c3)nc2n1] | (S)-4-((3,3-difluoropiperidin-1- yl)methyl)-N-(3-(1-((2-ethyl- 2H-pyrazolo[3,4-b]pyrazin-6-yl) amino)ethyl)phenyl)-3- methylbenzamide |
| 475 | 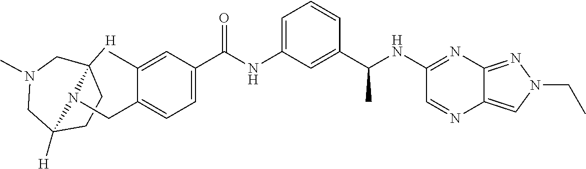 [SMILES: CCn1cc2ncc(N[C@@H](C)c3cccc(NC(=O)c4ccc(CN5[C@@H]6CC[C@H]5CN(C)C6)c(C)c4)c3)nc2n1] | N-(3-((S)-1-((2-ethyl-2H- pyrazolo[3,4-b]pyrazin-6-yl) amino)ethyl)phenyl)-3-methyl- 4-(((1R,5S)-3-methyl-3,8- diazabicyclo[3.2.1]octan-8-yl) methyl)benzamide |
| 476 | 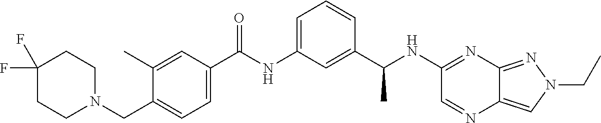 [SMILES: CCn1cc2ncc(N[C@@H](C)c3cccc(NC(=O)c4ccc(CN5CCC(F)(F)CC5)c(C)c4)c3)nc2n1] | (S)-4-((4,4-difluoropiperidin-1- yl)methyl)-N-(3-(1-((2-ethyl- 2H-pyrazolo[3,4-b]pyrazin-6-yl) amino)ethyl)phenyl)-3- methylbenzamide |
| 477 | 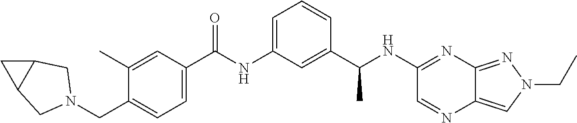 [SMILES: CCn1cc2ncc(N[C@@H](C)c3cccc(NC(=O)c4ccc(CN5CC6CC6C5)c(C)c4)c3)nc2n1] | 4-((3-azabicyclo[3.1.0]hexan-3- yl)methyl)-N-(3-((5)-1-((2- ethyl-2H-pyrazolo[3,4-b] pyrazin-6-yl)amino)ethyl) phenyl)-3-methylbenzamide |
| 478 | 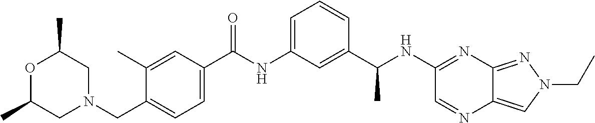 [SMILES: CCn1cc2ncc(N[C@@H](C)c3cccc(NC(=O)c4ccc(CN5C[C@@H](C)O[C@@H](C)C5)c(C)c4)c3)nc2n1] | 4-(((2S,6R)-2,6- dimethylmorpholino)methyl)-N- (3-((S)-1-((2-ethyl-2H- pyrazolo[3,4-b]pyrazin-6-yl) amino)ethyl)phenyl)-3- methylbenzamide |
| 479 | 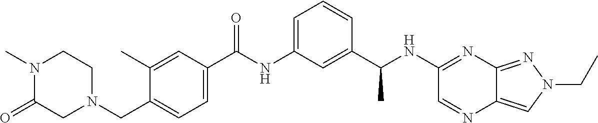 [SMILES: CCn1cc2ncc(N[C@@H](C)c3cccc(NC(=O)c4ccc(CN5CCN(C)C(=O)C5)c(C)c4)c3)nc2n1] | (S)-N-(3-(1-((2-ethyl-2H- pyrazolo[3,4-b]pyrazin-6-yl) amino)ethyl)phenyl)-3-methyl- 4-((4-methyl-3-oxopiperazin-1- yl)methyl)benzamide |
| 480 | 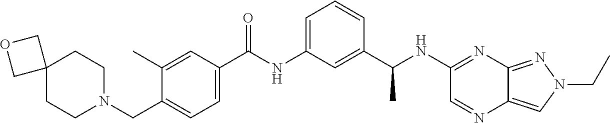 [SMILES: CCn1cc2ncc(N[C@@H](C)c3cccc(NC(=O)c4ccc(CN5CCC6(CC5)COC6)c(C)c4)c3)nc2n1] | (S)-4-((2-oxa-7- azaspiro[3.5]nonan-7-yl)methyl)- N-(3-(1-((2-ethyl-2H- pyrazolo[3,4-b]pyrazin-6-yl) amino)ethyl)phenyl)-3- methylbenzamide |
| 481 | 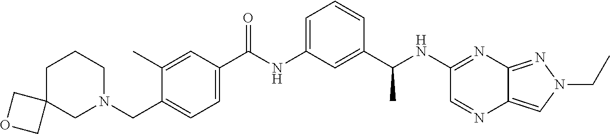 [SMILES: CCn1cc2ncc(N[C@@H](C)c3cccc(NC(=O)c4ccc(CN5CCCC6(COC6)C5)c(C)c4)c3)nc2n1] | (S)-4-((2-oxa-6- azaspiro[3.5]nonan-6-yl)methyl)- N-(3-(1-((2-ethyl-2H- pyrazolo[3,4-b]pyrazin-6-yl) amino)ethyl)phenyl)-3- methylbenzamide |
| 482 | 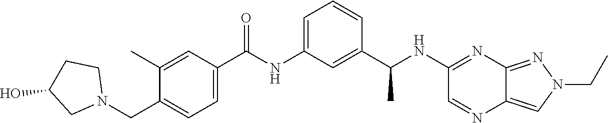 [SMILES: CCn1cc2ncc(N[C@@H](C)c3cccc(NC(=O)c4ccc(CN5CC[C@@H](O)C5)c(C)c4)c3)nc2n1] | N-(3-((S)-1-((2-ethyl-2H- pyrazolo[3,4-b]pyrazin-6-yl) annno)ethyl)phenyl)-4-(((R)-3- hydroxypyrrolidin-1-yl)methyl)- 3-methylbenzamide |
| 483 | 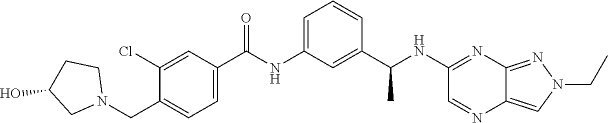 [SMILES: CCn1cc2ncc(N[C@@H](C)c3cccc(NC(=O)c4ccc(CN5CC[C@@H](O)C5)c(Cl)c4)c3)nc2n1] | 3-chloro-N-(3-((S)-1-((2-ethyl- 2H-pyrazolo[3,4-b]pyrazin-6-yl) amino)ethyl)phenyl)-4-(((R)-3- hydroxypyrrolidin-1-yl)methyl) benzamide |
| 484 | 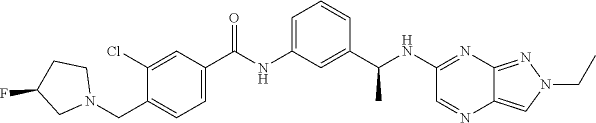 [SMILES: CCn1cc2ncc(N[C@@H](C)c3cccc(NC(=O)c4ccc(CN5CC[C@H](F)C5)c(Cl)c4)c3)nc2n1] | 3-chloro-N-(3-((S)-1-((2-ethyl- 2H-pyrazolo[3,4-b]pyrazin-6-yl) amino)ethyl)phenyl)-4-(((S)-3- fluoropyrrolidin-1-yl)methyl) benzamide |
| 485 | 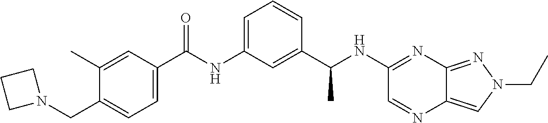 [SMILES: CCn1cc2ncc(N[C@@H](C)c3cccc(NC(=O)c4ccc(CN5CCC5)c(C)c4)c3)nc2n1] | (S)-4-(azetidin-1-ylmethyl)-N- (3-(1-((2-ethyl-2H-pyrazolo[3,4- b]pyrazin-6-yl)amino)ethyl) phenyl)-3-methylbenzamide |
| 486 | 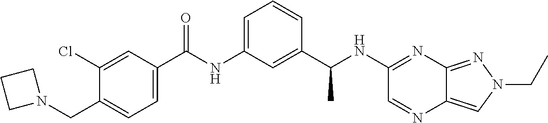 [SMILES: CCn1cc2ncc(N[C@@H](C)c3cccc(NC(=O)c4ccc(CN5CCC5)c(Cl)c4)c3)nc2n1] | (S)-4-(azetidin-1-ylmethyl)-3- chloro-N-(3-(1-((2-ethyl-2H- pyrazolo[3,4-b]pyrazin-6-yl) amino)ethyl)phenyl)benzamide |
| 487 | 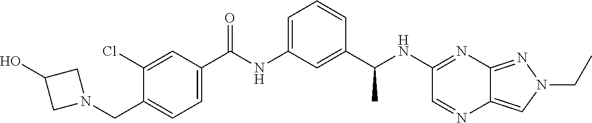 [SMILES: CCn1cc2ncc(N[C@@H](C)c3cccc(NC(=O)c4ccc(CN5CC(O)C5)c(Cl)c4)c3)nc2n1] | (S)-3-chloro-N-(3-(1-((2-ethyl- 2H-pyrazolo[3,4-b]pyrazin-6-yl) amino)ethyl)phenyl)-4-((3- hydroxyazetidin-1-yl)methyl) benzamide |
| 488 | 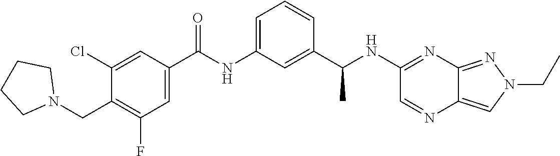 [SMILES: CCn1cc2ncc(N[C@@H](C)c3cccc(NC(=O)c4cc(F)c(CN5CCCC5)c(Cl)c4)c3)nc2n1] | (S)-3-chloro-N-(3-(1-((2-ethyl- 2H-pyrazolo[3,4-b]pyrazin-6-yl) amino)ethyl)phenyl)-5-fluoro-4- (pyrrolidin-1-ylmethyl) benzamide |
| 489 | 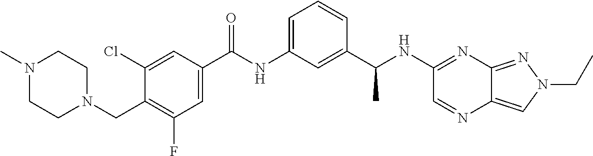 [SMILES: CCn1cc2ncc(N[C@@H](C)c3cccc(NC(=O)c4cc(F)c(CN5CCN(C)CC5)c(Cl)c4)c3)nc2n1] | (S)-3-chloro-N-(3-(1-((2-ethyl- 2H-pyrazolo[3,4-b]pyrazin-6-yl) amino)ethyl)phenyl)-5-fluoro-4- ((4-methylpiperazin-1-yl) methyl)benzamide |
| 490 | 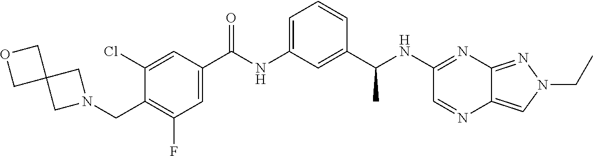 [SMILES: CCn1cc2ncc(N[C@@H](C)c3cccc(NC(=O)c4cc(F)c(CN5CC6(COC6)C5)c(Cl)c4)c3)nc2n1] | (S)-4-((2-oxa-6- azaspiro[3.3]heptan-6-yl) methyl)-3-chloro-N-(3-(1-((2- ethyl-2H-pyrazolo[3,4-b] pyrazin-6-yl)amino)ethyl) phenyl)-5-fluorobenzamide |
| 491 | 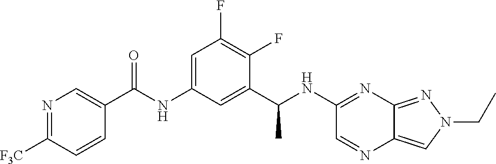 [SMILES: CCn1cc2ncc(N[C@@H](C)c3cc(NC(=O)c4ccc(C(F)(F)F)nc4)cc(F)c3F)nc2n1] | (S)-N-(3-(1-((2-ethyl-2H- pyrazolo[3,4-b]pyrazin-6-yl) amino)ethyl)-4,5- difluorophenyl)-6- (trifluoromethyl)nicotinamide |
| 492 | 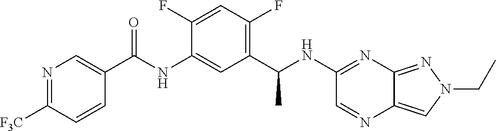 [SMILES: CCn1cc2ncc(N[C@@H](C)c3cc(NC(=O)c4ccc(C(F)(F)F)nc4)c(F)cc3F)nc2n1] | (S)-N-(5-(1-((2-ethyl-2H- pyrazolo[3,4-b]pyrazin-6-yl) amino)ethyl)-2,4- difluorophenyl)-6- (trifluoromethyl)nicotinamide |
| 493 | 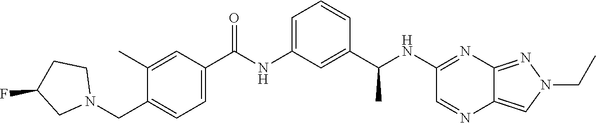 [SMILES: CCn1cc2ncc(N[C@@H](C)c3cccc(NC(=O)c4ccc(CN5CC[C@H](F)C5)c(C)c4)c3)nc2n1] | N-(3-((S)-1-((2-ethyl-2H- pyrazolo[3,4-b]pyrazin-6-yl) amino)ethyl)phenyl)-4-(((S)-3- fluoropyrrolidin-1-yl)methyl)-3- methylbenzamide |
| 494 | 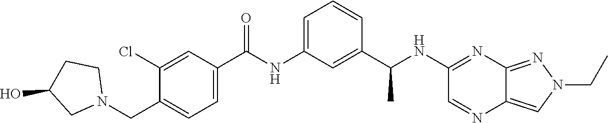 [SMILES: CCn1cc2ncc(N[C@@H](C)c3cccc(NC(=O)c4ccc(CN5CC[C@H](O)C5)c(Cl)c4)c3)nc2n1] | 3-chloro-N-(3-((5)-1-((2-ethyl- 2H-pyrazolo[3,4-b]pyrazin-6-yl) amino)ethyl)phenyl)-4-(((S)-3- hydroxypyrrolidin-1-yl)methyl) benzamide |
| 495 | 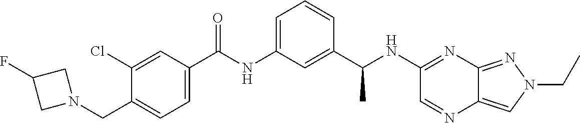 [SMILES: CCn1cc2ncc(N[C@@H](C)c3cccc(NC(=O)c4ccc(CN5CC(F)C5)c(Cl)c4)c3)nc2n1] | (S)-3-chloro-N-(3-(1-((2-ethyl- 2H-pyrazolo[3,4-b]pyrazin-6-yl) amino)ethyl)phenyl)-4-((3- fluoroazetidin-1-yl)methyl) benzamide |
| 496 | 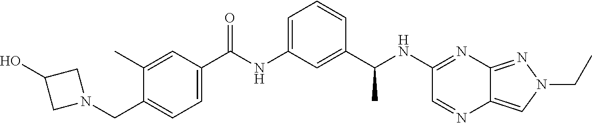 [SMILES: CCn1cc2ncc(N[C@@H](C)c3cccc(NC(=O)c4ccc(CN5CC(O)C5)c(C)c4)c3)nc2n1] | (S)-N-(3-(1-((2-ethyl-2H- pyrazolo[3,4-b]pyrazin-6-yl) amino)ethyl)phenyl)-4-((3- hydroxyazetidin-1-yl)methyl)-3- methylbenzamide |
| 497 | 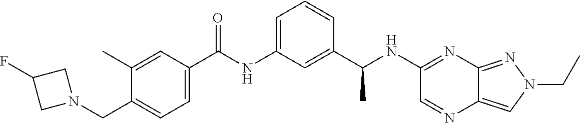 [SMILES: CCn1cc2ncc(N[C@@H](C)c3cccc(NC(=O)c4ccc(CN5CC(F)C5)c(C)c4)c3)nc2n1] | (S)-N-(3-(1-((2-ethyl-2H- pyrazolo[3,4-b]pyrazin-6-yl) amino)ethyl)phenyl)-4-((3- fluoroazetidin-1-yl)methyl)-3- methylbenzamide |
| 498 | 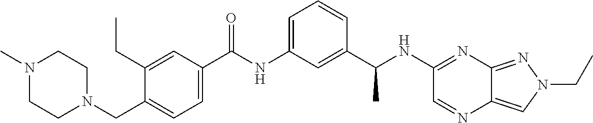 [SMILES: CCc1cc(C(=O)Nc2cccc([C@H](C)Nc3cnc4cn(CC)nc4n3)c2)ccc1CN1CCN(C)CC1] | (S)-3-ethyl-N-(3-(1-((2-ethyl- 2H-pyrazolo[3,4-b]pyrazin-6-yl) amino)ethyl)phenyl)-4-((4- methylpiperazin-1-yl)methyl) benzamide |
| 499 | 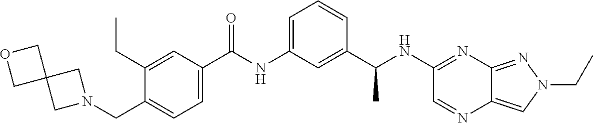 [SMILES: CCc1cc(C(=O)Nc2cccc([C@H](C)Nc3cnc4cn(CC)nc4n3)c2)ccc1CN1CC2(COC2)C1] | (S)-4-((2-oxa-6- azaspiro[3,3]heptan-6-yl) methyl)-3-ethyl-N-(3-(1-((2- ethyl-2H-pyrazolo[3,4-b] pyrazin-6-yl)amino)ethyl) phenyl)benzamide |
| 500 | 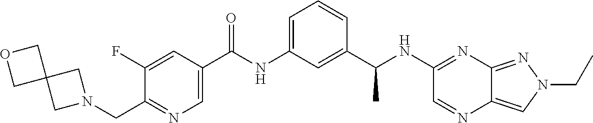 [SMILES: CCn1cc2ncc(N[C@@H](C)c3cccc(NC(=O)c4cnc(CN5CC6(COC6)C5)c(F)c4)c3)nc2n1] | (S)-6-((2-oxa-6- azaspiro[3.3]heptan-6-yl) methyl)-N-(3-(1-((2-ethyl-2H- pyrazolo[3,4-b]pyrazin-6-yl) amino)ethyl)phenyl)-5- fluoronicotinamide |
| 501 | 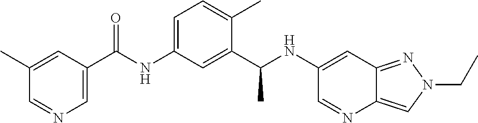 [SMILES: CCn1cc2ncc(N[C@@H](C)c3cc(NC(=O)c4cncc(C)c4)ccc3C)cc2n1] | (S)-N-(3-(1-((2-ethyl-2H- pyrazolo[4,3-b]pyridin-6-yl) amino)ethyl)-4-methylphenyl)- 5-methylnicotinamide |
| 502 | 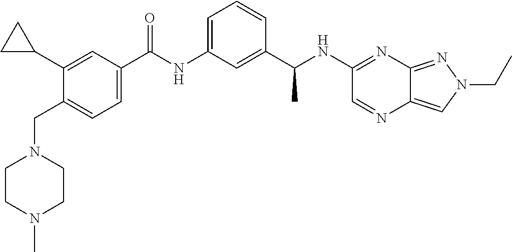 [SMILES: CCn1cc2ncc(N[C@@H](C)c3cccc(NC(=O)c4ccc(CN5CCN(C)CC5)c(C5CC5)c4)c3)nc2n1] | (S)-3-cyclopropyl-N-(3-(1-((2- ethyl-2H-pyrazolo[3,4-b] pyrazin-6-yl)amino)ethyl) phenyl)-4-((4-methylpiperazin- 1-yl)methyl)benzamide |
| 503 | 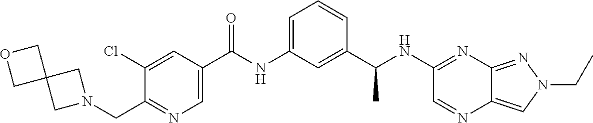 [SMILES: CCn1cc2ncc(N[C@@H](C)c3cccc(NC(=O)c4cnc(CN5CC6(COC6)C5)c(Cl)c4)c3)nc2n1] | (S)-6-((2-oxa-6- azaspiro[3.3]heptan-6-yl) methyl)-5-chloro-N-(3-(1-((2- ethyl-2H-pyrazolo[3,4-b] pyrazin-6-yl)amino)ethyl) phenyl)nicotinamide |
| 504 | 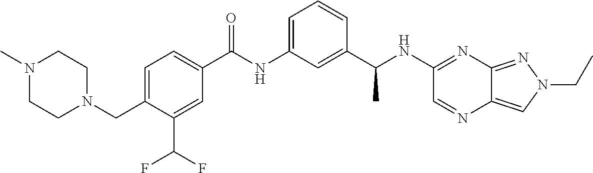 [SMILES: CCn1cc2ncc(N[C@@H](C)c3cccc(NC(=O)c4ccc(CN5CCN(C)CC5)c(C(F)F)c4)c3)nc2n1] | (S)-3-(difluoromethyl)-N-(3-(1- ((2-ethyl-2H-pyrazolo[3,4-b] pyrazin-6-yl)amino)ethyl) phenyl)-4-((4-methylpiperazin- 1-yl)methyl)benzamide |
| 505 | 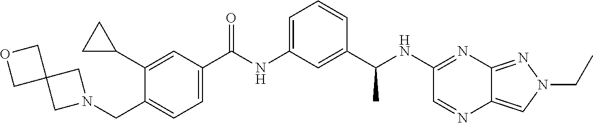 [SMILES: CCn1cc2ncc(N[C@@H](C)c3cccc(NC(=O)c4ccc(CN5CC6(COC6)C5)c(C5CC5)c4)c3)nc2n1] | (S)-4-((2-oxa-6- azaspiro[3.3]heptan-6-yl) methyl)-3-cyclopropyl-N-(3-(1- ((2-ethyl-2H-pyrazolo[3,4-b] pyrazin-6-yl)amino)ethyl) phenyl)benzamide |
| 506 | 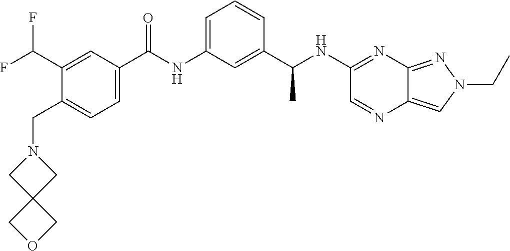 [SMILES: CCn1cc2ncc(N[C@@H](C)c3cccc(NC(=O)c4ccc(CN5CC6(COC6)C5)c(C(F)F)c4)c3)nc2n1] | (S)-4-((2-oxa-6- azaspiro[3.3]heptan-6-yl) methyl)-3-(difluoromethyl)-N- (3-(1-((2-ethyl-2H-pyrazolo[3,4- b]pyrazin-6-yl)amino)ethyl) phenyl)benzamide |
| 507 | 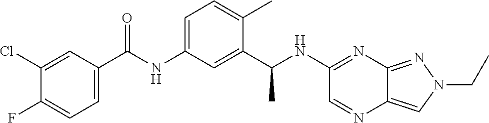 [SMILES: CCn1cc2ncc(N[C@@H](C)c3cc(NC(=O)c4ccc(F)c(Cl)c4)ccc3C)nc2n1] | (S)-3-chloro-N-(3-(1-((2-ethyl- 2H-pyrazolo[3,4-b]pyrazin-6-yl) amino)ethyl)-4-methylphenyl)- 4-fluorobenzamide |
| 508 | 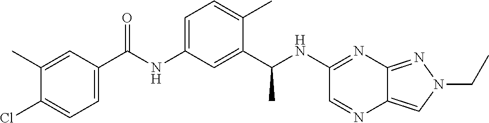 [SMILES: CCn1cc2ncc(N[C@@H](C)c3cc(NC(=O)c4ccc(Cl)c(C)c4)ccc3C)nc2n1] | (S)-4-chloro-N-(3-(1-((2-ethyl- 2H-pyrazolo[3,4-b]pyrazin-6-yl) amino)ethyl)-4-methylphenyl)- 3-methylbenzamide |
| 509 | 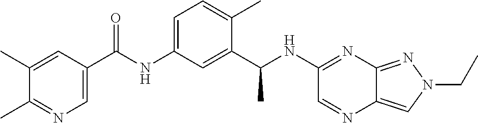 [SMILES: CCn1cc2ncc(N[C@@H](C)c3cc(NC(=O)c4cnc(C)c(C)c4)ccc3C)nc2n1] | (S)-N-(3-(1-((2-ethyl-2H- pyrazolo[3,4-b]pyrazin-6-yl) amino)ethyl)-4-methylphenyl)- 5,6-dimethylnicotinamide |
| 510 | 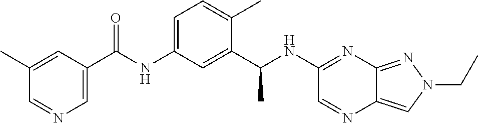 [SMILES: CCn1cc2ncc(N[C@@H](C)c3cc(NC(=O)c4cncc(C)c4)ccc3C)nc2n1] | (S)-N-(3-(1-((2-ethyl-2H- pyrazolo[3,4-b]pyrazin-6-yl) amino)ethyl)-4-methylphenyl)- 5-methylnicotinamide |
| 511 | 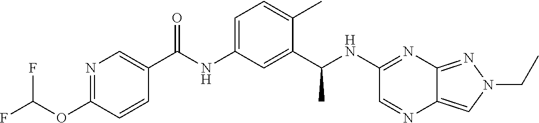 [SMILES: CCn1cc2ncc(N[C@@H](C)c3cc(NC(=O)c4ccc(OC(F)F)nc4)ccc3C)nc2n1] | (S)-6-(difluoromethoxy)-N-(3- (1-((2-ethyl-2H-pyrazolo[3,4-b] pyrazin-6-yl)amino)ethyl)-4- methylphenyl)nicotinamide |
| 512 | 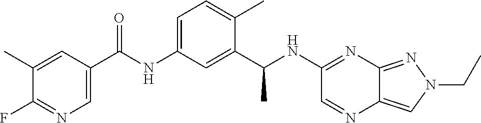 [SMILES: CCn1cc2ncc(N[C@@H](C)c3cc(NC(=O)c4cnc(F)c(C)c4)ccc3C)nc2n1] | (S)-N-(3-(1-((2-ethyl-2H- pyrazolo[3,4-b]pyrazin-6-yl) amino)ethyl)-4-methylphenyl)- 6-fluoro-5-methylnicotinamide |
| 513 | 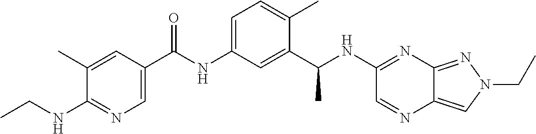 [SMILES: CCNc1ncc(C(=O)Nc2ccc(C)c([C@H](C)Nc3cnc4cn(CC)nc4n3)c2)cc1C] | (S)-N-(3-(1-((2-ethyl-2H- pyrazolo[3,4-b]pyrazin-6-yl) amino)ethyl)-4-methylphenyl)- 6-(ethylamino)-5- methylnicotinamide |
| 514 | 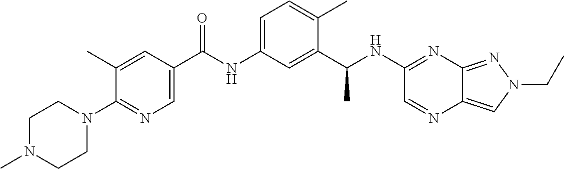 [SMILES: CCn1cc2ncc(N[C@@H](C)c3cc(NC(=O)c4cnc(N5CCN(C)CC5)c(C)c4)ccc3C)nc2n1] | (S)-N-(3-(1-((2-ethyl-2H- pyrazolo[3,4-b]pyrazin-6-yl) amino)ethyl)-4-methylphenyl)- 5-methyl-6-(4-methylpiperazin- 1-yl)nicotinamide |
| 515 | 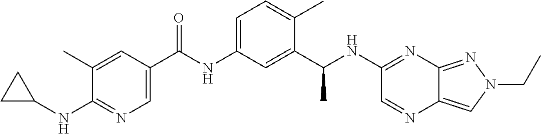 [SMILES: CCn1cc2ncc(N[C@@H](C)c3cc(NC(=O)c4cnc(NC5CC5)c(C)c4)ccc3C)nc2n1] | (S)-6-(cyclopropylamino)-N-(3- (1-((2-ethyl-2H-pyrazolo[3,4-b] pyrazin-6-yl)amino)ethyl)-4- methylphenyl)-5- methylnicotinamide |
| 516 | 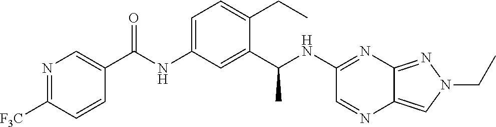 [SMILES: CCc1ccc(NC(=O)c2ccc(C(F)(F)F)nc2)cc1[C@H](C)Nc1cnc2cn(CC)nc2n1] | (S)-N-(4-ethyl-3-(1-((2-ethyl- 2H-pyrazolo[3,4-b]pyrazin-6-yl) amino)ethyl)phenyl)-6- (trifluoromethyl)nicotinamide |
| 517 | 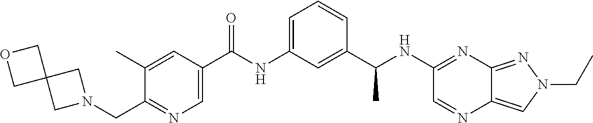 [SMILES: CCn1cc2ncc(N[C@@H](C)c3cccc(NC(=O)c4cnc(CN5CC6(COC6)C5)c(C)c4)c3)nc2n1] | (S)-6-((2-oxa-6- azaspiro[3.3]heptan-6-yl) methyl)-N-(3-(1-((2-ethyl-2H- pyrazolo[3,4-b]pyrazin-6-yl) amino)ethyl)phenyl)-5- methylnicotinamide |
| 518 | 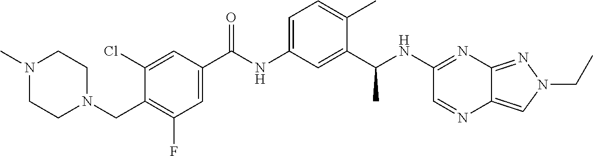 [SMILES: CCn1cc2ncc(N[C@@H](C)c3cc(NC(=O)c4cc(F)c(CN5CCN(C)CC5)c(Cl)c4)ccc3C)nc2n1] | (S)-3-chloro-N-(3-(1-((2-ethyl- 2H-pyrazolo[3,4-b]pyrazin-6-yl) amino)ethyl)-4-methylphenyl)- 5-fluoro-4-((4-methylpiperazin- 1-yl)methyl)benzamide |
| 519 | 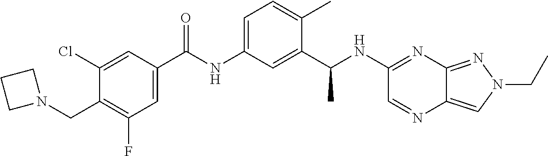 [SMILES: CCn1cc2ncc(N[C@@H](C)c3cc(NC(=O)c4cc(F)c(CN5CCC5)c(Cl)c4)ccc3C)nc2n1] | (S)-4-(azetidin-1-ylmethyl)-3- chloro-N-(3-(1-((2-ethyl-2H- pyrazolo[3,4-b]pyrazin-6-yl) amino)ethyl)-4-methylphenyl)- 5-fluorobenzamide |
| 520 | 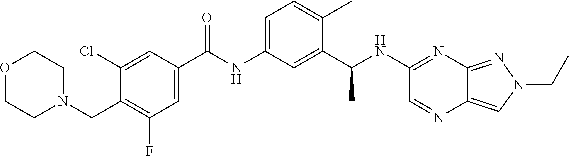 [SMILES: CCn1cc2ncc(N[C@@H](C)c3cc(NC(=O)c4cc(F)c(CN5CCOCC5)c(Cl)c4)ccc3C)nc2n1] | (S)-3-chloro-N-(3-(1-((2-ethyl- 2H-pyrazolo[3,4-b]pyrazin-6-yl) amino)ethyl)-4-methylphenyl)- 5-fluoro-4-(morpholinomethyl) benzamide |
| 521 | 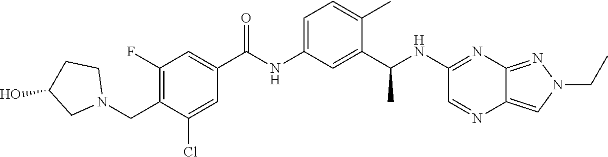 [SMILES: CCn1cc2ncc(N[C@@H](C)c3cc(NC(=O)c4cc(F)c(CN5CC[C@@H](O)C5)c(Cl)c4)ccc3C)nc2n1] | 3-chloro-N-(3-((S)-1-((2-ethyl- 2H-pyrazolo[3,4-b]pyrazin-6-yl) amino)ethyl)-4-methylphenyl)- 5-fluoro-4-(((R)-3- hydroxypyrrolidin-1-yl)methyl) benzamide |
| 522 | 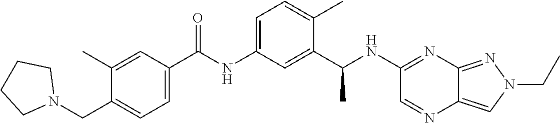 [SMILES: CCn1cc2ncc(N[C@@H](C)c3cc(NC(=O)c4ccc(CN5CCCC5)c(C)c4)ccc3C)nc2n1] | (S)-N-(3-(1-((2-ethyl-2H- pyrazolo[3,4-b]pyrazin-6-yl) amino)ethyl)-4-methylphenyl)- 3-methyl-4-(pyrrolidin-1- ylmethyl)benzamide |
| 523 | 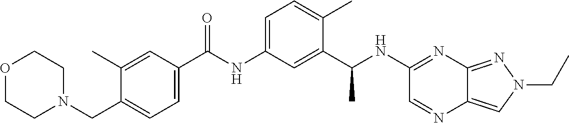 [SMILES: CCn1cc2ncc(N[C@@H](C)c3cc(NC(=O)c4ccc(CN5CCOCC5)c(C)c4)ccc3C)nc2n1] | (S)-N-(3-(1-((2-ethyl-2H- pyrazolo[3,4-b]pyrazin-6-yl) amino)ethyl)-4-methylphenyl)- 3-methyl-4-(morpholinomethyl) benzamide |
| 524 | 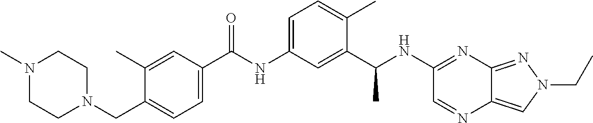 [SMILES: CCn1cc2ncc(N[C@@H](C)c3cc(NC(=O)c4ccc(CN5CCN(C)CC5)c(C)c4)ccc3C)nc2n1] | (S)-N-(3-(1-((2-ethyl-2H- pyrazolo[3,4-b]pyrazin-6-yl) amino)ethyl)-4-methylphenyl)- 3-methyl-4-((4-methylpiperazin- 1-yl)methyl)benzamide |
| 525 | 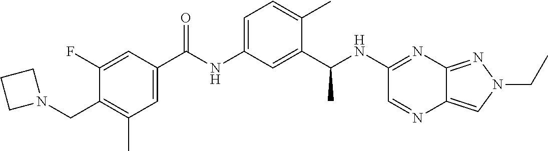 [SMILES: CCn1cc2ncc(N[C@@H](C)c3cc(NC(=O)c4cc(C)c(CN5CCC5)c(F)c4)ccc3C)nc2n1] | (S)-4-(azetidin-1-ylmethyl)-N- (3-(1-((2-ethyl-2H-pyrazolo[3,4- b]pyrazin-6-yl)amino)ethyl)-4- methylphenyl)-3-fluoro-5- methylbenzamide |
| 526 | 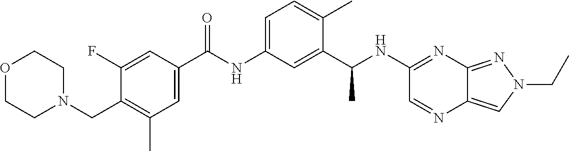 [SMILES: CCn1cc2ncc(N[C@@H](C)c3cc(NC(=O)c4cc(C)c(CN5CCOCC5)c(F)c4)ccc3C)nc2n1] | (S)-N-(3-(1-((2-ethyl-2H- pyrazolo[3,4-b]pyrazin-6-yl) amino)ethyl)-4-methylphenyl)- 3-fluoro-5-methyl-4- (morpholinomethyl)benzamide |
| 527 | 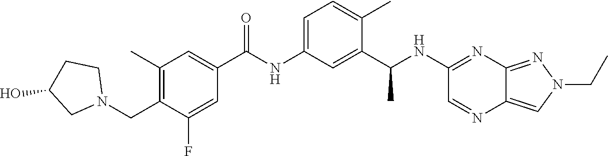 [SMILES: CCn1cc2ncc(N[C@@H](C)c3cc(NC(=O)c4cc(C)c(CN5CC[C@@H](O)C5)c(F)c4)ccc3C)nc2n1] | N-(3-((S)-1-((2-ethyl-2H- pvrazolo[3,4-b]pyrazin-6-yl) amino)ethyl)-4-methylphenyl)- 3-fluoro-4-(((R)-3- hydroxypyrrolidin-1-yl)methyl)- 5-methylbenzamide |
| 528 | 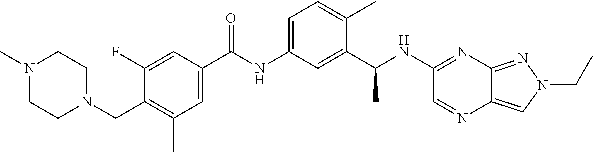 [SMILES: CCn1cc2ncc(N[C@@H](C)c3cc(NC(=O)c4cc(C)c(CN5CCN(C)CC5)c(F)c4)ccc3C)nc2n1] | (S)-N-(3-(1-((2-ethyl-2H- pyrazolo[3,4-b]pyrazin-6-yl) amino)ethyl)-4-methylphenyl)- 3-fluoro-5-methyl-4-((4- methylpiperazin-1-yl)methyl) benzamide |
| 529 | 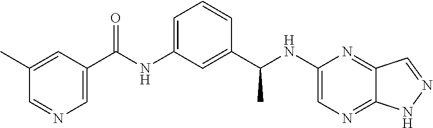 [SMILES: Cc1cncc(C(=O)Nc2cccc([C@H](C)Nc3cnc4[nH]ncc4n3)c2)c1] | (S)-N-(3-(1-((1H-pyrazolo[3,4- b]pyrazin-5-yl)amino)ethyl) phenyl)-5-methylnicotinamide |
| 530 | 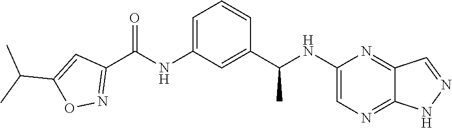 [SMILES: CC(C)c1cc(C(=O)Nc2cccc([C@H](C)Nc3cnc4[nH]ncc4n3)c2)no1] | (S)-N-(3-(1-((1H-pyrazolo[3,4- b]pyrazin-5-yl)amino)ethyl) phenyl)-5-isopropylisoxazole-3- carboxamide |
| 531 | 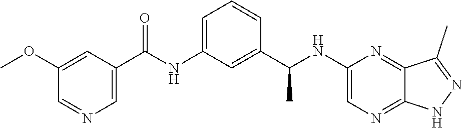 [SMILES: COc1cncc(C(=O)Nc2cccc([C@H](C)Nc3cnc4[nH]nc(C)c4n3)c2)c1] | (S)-5-methoxy-N-(3-(1-((3- methyl-1H-pyrazolo[3,4-b] pyrazin-5-yl)amino)ethyl) phenyl)nicotinamide |
| 532 | 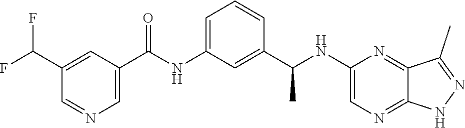 [SMILES: Cc1n[nH]c2ncc(N[C@@H](C)c3cccc(NC(=O)c4cncc(C(F)F)c4)c3)nc12] | (S)-5-(difluoromethyl)-N-(3-(1- ((3-methyl-1H-pyrazolo[3,4-b] pyrazin-5-yl)amino)ethyl) phenyl)nicotinamide |
| 533 | 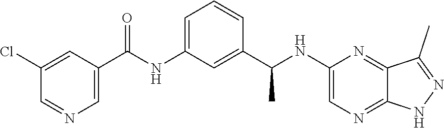 [SMILES: Cc1n[nH]c2ncc(N[C@@H](C)c3cccc(NC(=O)c4cncc(Cl)c4)c3)nc12] | (S)-5-chloro-N-(3-(1-((3- methyl-1H-pyrazolo[3,4-b] pyrazin-5-yl)amino)ethyl) phenyl)nicotinamide |
| 534 | 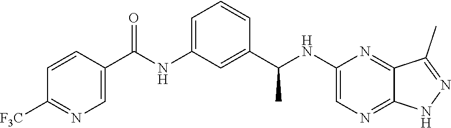 [SMILES: Cc1n[nH]c2ncc(N[C@@H](C)c3cccc(NC(=O)c4ccc(C(F)(F)F)nc4)c3)nc12] | (S)-N-(3-(1-((3-methyl-1H- pyrazolo[3,4-b]pyrazin-5-yl) amino)ethyl)phenyl)-6- (trifluoromethyl)nicotinamide |
| 535 | 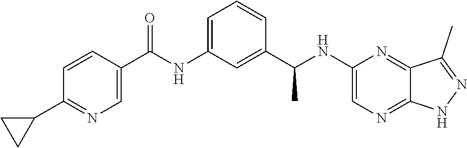 [SMILES: Cc1n[nH]c2ncc(N[C@@H](C)c3cccc(NC(=O)c4ccc(C5CC5)nc4)c3)nc12] | (S)-6-cyclopropyl-N-(3-(1-((3- methyl-1H-pyrazolo[3,4-b] pyrazin-5-yl)amino)ethyl) phenyl)nicotinamide |
| 536 | 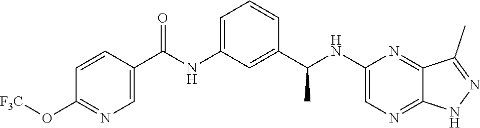 [SMILES: Cc1n[nH]c2ncc(N[C@@H](C)c3cccc(NC(=O)c4ccc(OC(F)(F)F)nc4)c3)nc12] | (S)-N-(3-(1-((3-methyl-1H- pyrazolo[3,4-b]pyrazin-5-yl) amino)ethyl)phenyl)-6- (trifluoromethoxy)nicotinamide |
| 537 | 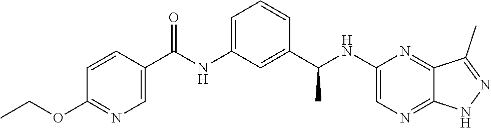 [SMILES: CCOc1ccc(C(=O)Nc2cccc([C@H](C)Nc3cnc4[nH]nc(C)c4n3)c2)cn1] | (S)-6-ethoxy-N-(3-(1-((3- methyl-1H-pyrazolo[3,4-b] pyrazin-5-yl)amino)ethyl) phenyl)nicotinamide |
| 538 | 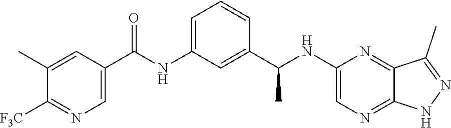 [SMILES: Cc1cc(C(=O)Nc2cccc([C@H](C)Nc3cnc4[nH]nc(C)c4n3)c2)cnc1C(F)(F)F] | (S)-5-methyl-N-(3-(1-((3- methyl-1H-pyrazolo[3,4-b] pyrazin-5-yl)amino)ethyl) phenyl)-6-(trifluoromethyl) nicotinamide |
| 539 | 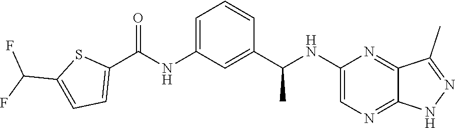 [SMILES: Cc1n[nH]c2ncc(N[C@@H](C)c3cccc(NC(=O)c4ccc(C(F)F)s4)c3)nc12] | (S)-5-(difluoromethyl)-N-(3-(1- ((3-methyl-1H-pyrazolo[3,4-b] pyrazin-5-yl)amino)ethyl) phenyl)thiophene-2- carboxamide |
| 540 | 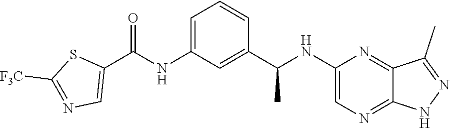 [SMILES: Cc1n[nH]c2ncc(N[C@@H](C)c3cccc(NC(=O)c4cnc(C(F)(F)F)s4)c3)nc12] | (S)-N-(3-(1-((3-methyl-1H- pyrazolo[3,4-b]pyrazin-5-yl) amino)ethyl)phenyl)-2- (trifluoromethyl)thiazole-5- carboxamide |
| 541 | 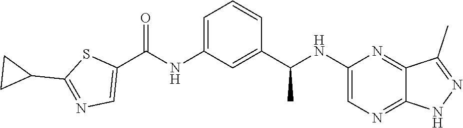 [SMILES: Cc1n[nH]c2ncc(N[C@@H](C)c3cccc(NC(=O)c4cnc(C5CC5)s4)c3)nc12] | (S)-2-cyclopropyl-N-(3-(1-((3- methyl-1H-pyrazolo[3,4-b] pyrazin-5-yl)amino)ethyl) phenyl)thiazole-5-carboxamide |
| 542 | 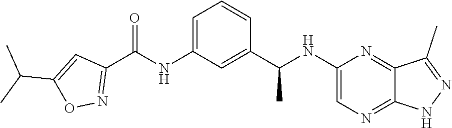 [SMILES: Cc1n[nH]c2ncc(N[C@@H](C)c3cccc(NC(=O)c4cc(C(C)C)on4)c3)nc12] | (S)-5-isopropyl-N-(3-(1-((3- methyl-1H-pyrazolo[3,4-b] pyrazin-5-yl)amino)ethyl) phenyl)isoxazole-3- carboxamide |
| 543 | 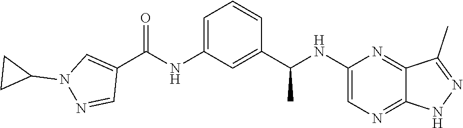 [SMILES: Cc1n[nH]c2ncc(N[C@@H](C)c3cccc(NC(=O)c4cnn(C5CC5)c4)c3)nc12] | (S)-1-cyclopropyl-N-(3-(1-((3- methyl-1H-pyrazolo[3,4-b] pyrazin-5-yl)amino)ethyl) phenyl)-1H-pyrazole-4- carboxamide |
| 544 | 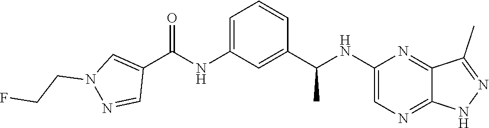 [SMILES: Cc1n[nH]c2ncc(N[C@@H](C)c3cccc(NC(=O)c4cnn(CCF)c4)c3)nc12] | (S)-1-(2-fluoroethyl)-N-(3-(1- ((3-methyl-1H-pyrazolo[3,4-b] pyrazin-5-yl)amino)ethyl) phenyl)-1H-pyrazole-4- carboxamide |
| 545 | 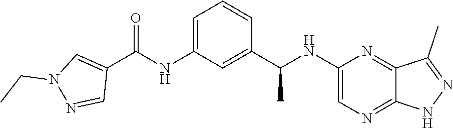 [SMILES: CCn1cc(C(=O)Nc2cccc([C@H](C)Nc3cnc4[nH]nc(C)c4n3)c2)cn1] | (S)-1-ethyl-N-(3-(1-((3-methyl- 1H-pyrazolo[3,4-b]pyrazin-5-yl) amino)ethyl)phenyl)-1H- pyrazole-4-carboxamide |
| 546 | 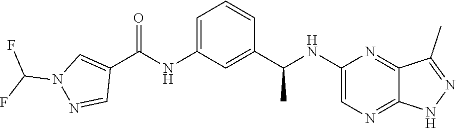 [SMILES: Cc1n[nH]c2ncc(N[C@@H](C)c3cccc(NC(=O)c4cnn(C(F)F)c4)c3)nc12] | (S)-1-(difluoromethyl)-N-(3-(1- ((3-methyl-1H-pyrazolo[3,4-b] pyrazin-5-yl)amino)ethyl) phenyl)-1H-pyrazole-4- carboxamide |
| 547 | 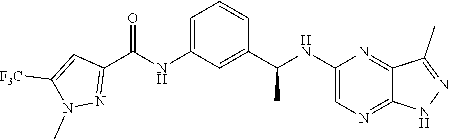 [SMILES: Cc1n[nH]c2ncc(N[C@@H](C)c3cccc(NC(=O)c4cc(C(F)(F)F)n(C)n4)c3)nc12] | (S)-1-methyl-N-(3-(1-((3- methyl-1H-pyrazolo[3,4-b] pyrazin-5-yl)amino)ethyl) phenyl)-5-(trifluoromethyl)-1H- pyrazole-3-carboxamide |
| 548 | 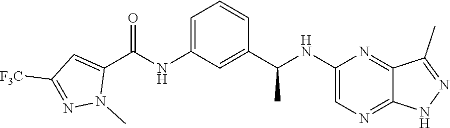 [SMILES: Cc1n[nH]c2ncc(N[C@@H](C)c3cccc(NC(=O)c4cc(C(F)(F)F)nn4C)c3)nc12] | (S)-1-methyl-N-(3-(1-((3- methyl-1H-pyrazolo[3,4-b] pyrazin-5-yl)amino)ethyl) phenyl)-3-(trifluoromethyl)-1H- pyrazole-5-carboxamide |
| 549 | 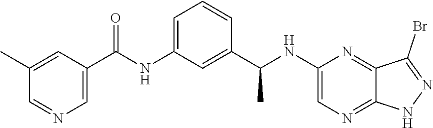 [SMILES: Cc1cncc(C(=O)Nc2cccc([C@H](C)Nc3cnc4[nH]nc(Br)c4n3)c2)c1] | (S)-N-(3-(1-((3-bromo-1H- pyrazolo[3,4-b]pyrazin-5-yl) amino)ethyl)phenyl)-5- methylnicotinamide |
| 550 | 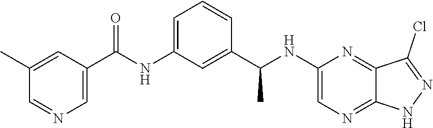 [SMILES: Cc1cncc(C(=O)Nc2cccc([C@H](C)Nc3cnc4[nH]nc(Cl)c4n3)c2)c1] | (S)-N-(3-(1-((3-chloro-1H- pyrazolo[3,4-b]pyrazin-5-yl) amino)ethyl)phenyl)-5- methylnicotinamide |
| 551 | 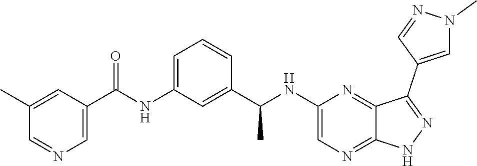 | (S)-5-methyl-N-(3-(1-((3-(1- methyl-1H-pyrazol-4-yl)-1H- pyrazolo[3,4-b]pyrazin-5-yl) amino)ethyl)phenyl) nicotinamide |
| 552 | 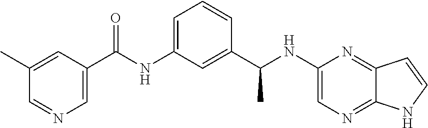 [SMILES: Cc1cncc(C(=O)Nc2cccc([C@H](C)Nc3cnc4[nH]ccc4n3)c2)c1] | (S)-N-(3-(1-((5H-pyrrolo[2,3-b] pyrazin-2-yl)amino)ethyl) phenyl)-5-methylnicotinamide |
| 553 | 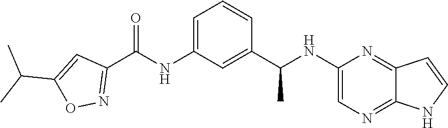 [SMILES: CC(C)c1cc(C(=O)Nc2cccc([C@H](C)Nc3cnc4[nH]ccc4n3)c2)no1] | (S)-N-(3-(1-((5H-pyrrolo[2,3-b] pyrazin-2-yl)amino)ethyl) phenyl)-5-isopropylisoxazole-3- carboxamide |
| 554 | 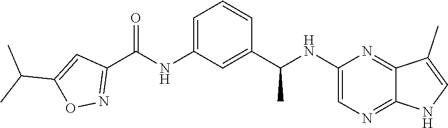 [SMILES: Cc1c[nH]c2ncc(N[C@@H](C)c3cccc(NC(=O)c4cc(C(C)C)on4)c3)nc12] | (S)-5-isopropyl-N-(3-(1-((7- methyl-5H-pyrrolo[2,3-b] pyrazin-2-yl)amino)ethyl) phenyl)isoxazole-3- carboxamide |
| 555 | 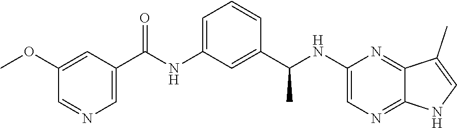 [SMILES: COc1cncc(C(=O)Nc2cccc([C@H](C)Nc3cnc4[nH]cc(C)c4n3)c2)c1] | (S)-5-methoxy-N-(3-(1-((7- methyl-5H-pyrrolo[2,3-b] pyrazin-2-yl)amino)ethyl) phenyl)nicotinamide |
| 556 | 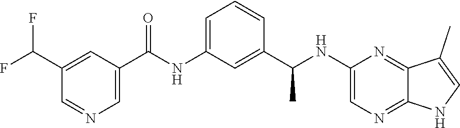 [SMILES: Cc1c[nH]c2ncc(N[C@@H](C)c3cccc(NC(=O)c4cncc(C(F)F)c4)c3)nc12] | (S)-5-(difluoromethyl)-N-(3-(1- ((7-methyl-5H-pyrrolo[2,3-6] pyrazin-2-yl)amino)ethyl) phenyl)nicotinamide |
| 557 | 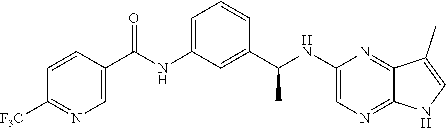 [SMILES: Cc1c[nH]c2ncc(N[C@@H](C)c3cccc(NC(=O)c4ccc(C(F)(F)F)nc4)c3)nc12] | (S)-N-(3-(1-((7-methyl-5H- pyrrolo[2,3-b]pyrazin-2-yl) amino)ethyl)phenyl)-6- (trifluoromethyl)nicotinamide |
| 558 | 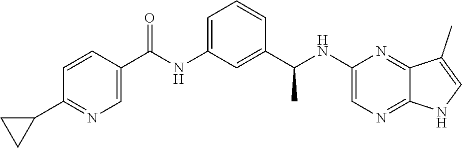 [SMILES: Cc1c[nH]c2ncc(N[C@@H](C)c3cccc(NC(=O)c4ccc(C5CC5)nc4)c3)nc12] | (S)-6-cyclopropyl-N-(3-(1-((7- methyl-5H-pyrrolo[2,3-b] pyrazin-2-yl)amino)ethyl) phenyl)nicotinamide |
| 559 | 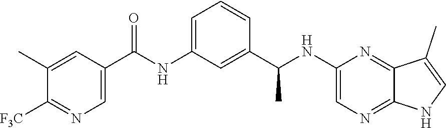 [SMILES: Cc1cc(C(=O)Nc2cccc([C@H](C)Nc3cnc4[nH]cc(C)c4n3)c2)cnc1C(F)(F)F] | (S)-5-methyl-N-(3-(1-((7- methyl-5H-pyrrolo[2,3-b] pyrazin-2-yl)amino)ethyl) phenyl)-6-(trifluoromethyl) nicotinamide |
| 560 | 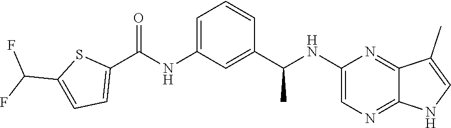 [SMILES: Cc1c[nH]c2ncc(N[C@@H](C)c3cccc(NC(=O)c4ccc(C(F)F)s4)c3)nc12] | (S)-5-(difluoromethyl)-N-(3-(1- ((7-methyl-5H-pyrrolo[2,3-b] pyrazin-2-yl)amino)ethyl) phenyl)thiophene-2- carboxamide |
| 561 | 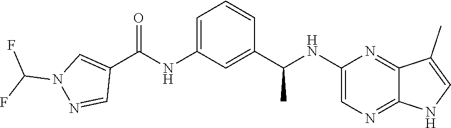 [SMILES: Cc1c[nH]c2ncc(N[C@@H](C)c3cccc(NC(=O)c4cnn(C(F)F)c4)c3)nc12] | (S)-1-(difluoromethyl)-N-(3-(1- ((7-methyl-5H-pyrrolo[2,3-b] pyrazin-2-yl)amino)ethyl) phenyl)-1H-pyrazole-4- carboxamide |
| 562 | 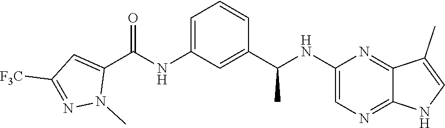 [SMILES: Cc1c[nH]c2ncc(N[C@@H](C)c3cccc(NC(=O)c4cc(C(F)(F)F)nn4C)c3)nc12] | (S)-1-methyl-N-(3-(1-((7- methyl-5H-pyrrolo[2,3-b] pyrazin-2-yl)amino)ethyl) phenyl)-3-(trifluoromethyl)-1H- pyrazole-5-carboxamide |
| 563 | 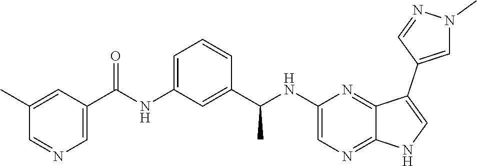 | (S)-5-methyl-N-(3-(1-((7-(1- methyl-H-pyrazol-4-yl)-5H- pyrrolo[2,3-b]pyrazin-2-yl) amino)ethyl)phenyl) nicotinamide |
| 564 | 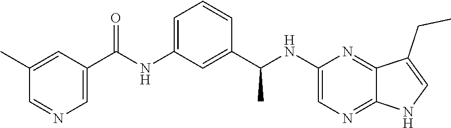 [SMILES: CCc1c[nH]c2ncc(N[C@@H](C)c3cccc(NC(=O)c4cncc(C)c4)c3)nc12] | (S)-N-(3-(1-((7-ethyl-5H- pyrrolo[2,3-b]pyrazin-2-yl) amino)ethyl)phenyl)-5- methylnicotinamide |
| 565 | 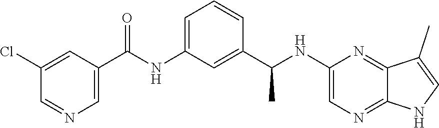 [SMILES: Cc1c[nH]c2ncc(N[C@@H](C)c3cccc(NC(=O)c4cncc(Cl)c4)c3)nc12] | (S)-5-chloro-N-(3-(1-((7- methyl-5H-pyrrolo[2,3-b] pyrazin-2-yl)amino)ethyl) phenyl)nicotinamide |
| 566 | 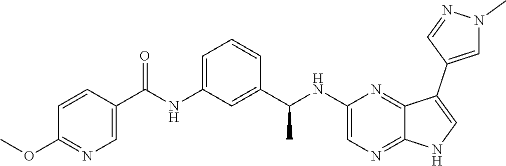 | (S)-6-methoxy-N-(3-(1-((7-(1- methyl-1H-pyrazol-4-yl)-5H- pyrrolo[2,3-b]pyrazin-2-yl) amino)ethyl)phenyl) nicotinamide |
| 567 | 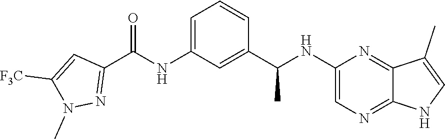 [SMILES: Cc1c[nH]c2ncc(N[C@@H](C)c3cccc(NC(=O)c4cc(C(F)(F)F)n(C)n4)c3)nc12] | (S)-1-methyl-N-(3-(1-((7- methyl-5H-pyrrolo[2,3-b] pyrazin-2-yl)amino)ethyl) phenyl)-5-(trifluoromethyl)-1H- pyrazole-3-carboxamide |
| 568 | 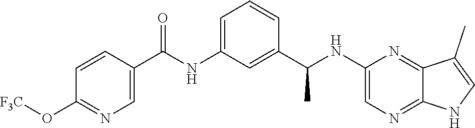 [SMILES: Cc1c[nH]c2ncc(N[C@@H](C)c3cccc(NC(=O)c4ccc(OC(F)(F)F)nc4)c3)nc12] | (S)-N-(3-(1-((7-methyl-5H- pyrrolo[2,3-b]pyrazin-2-yl) amino)ethyl)phenyl)-6- (trifluoromethoxy)nicotinamide |
| 569 | 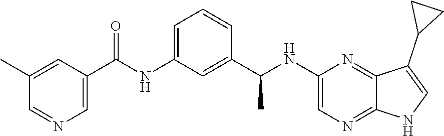 [SMILES: Cc1cncc(C(=O)Nc2cccc([C@H](C)Nc3cnc4[nH]cc(C5CC5)c4n3)c2)c1] | (S)-N-(3-(1-((7-cyclopropyl-5H- pyrrolo[2,3-b]pyrazin-2-yl) amino)ethyl)phenyl)-5- methylnicotinamide |
| 570 | 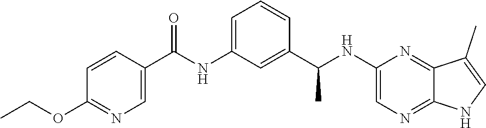 [SMILES: CCOc1ccc(C(=O)Nc2cccc([C@H](C)Nc3cnc4[nH]cc(C)c4n3)c2)cn1] | (S)-6-ethoxy-N-(3-(1-((7- methyl-5H-pyrrolo[2,3-b] pyrazin-2-yl)amino)ethyl) phenyl)nicotinamide |
| 571 | 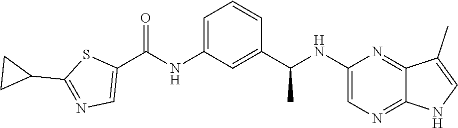 [SMILES: Cc1c[nH]c2ncc(N[C@@H](C)c3cccc(NC(=O)c4cnc(C5CC5)s4)c3)nc12] | (S)-2-cyclopropyl-N-(3-(1-((7- methyl-5H-pyrrolo[2,3-b] pyrazin-2-yl)amino)ethyl) phenyl)thiazole-5-carboxamide |
| 572 | 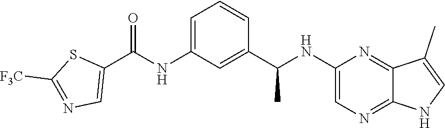 [SMILES: Cc1c[nH]c2ncc(N[C@@H](C)c3cccc(NC(=O)c4cnc(C(F)(F)F)s4)c3)nc12] | (S)-N-(3-(1-((7-methyl-5H- pyrrolo[2,3-b]pyrazin-2-yl) amino)ethyl)phenyl)-2- (trifluoromethyl)thiazole-5- carboxamide |
| 573 | 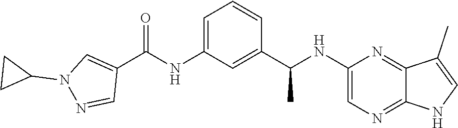 [SMILES: Cc1c[nH]c2ncc(N[C@@H](C)c3cccc(NC(=O)c4cnn(C5CC5)c4)c3)nc12] | (s)-1-cyclopropyl-N-(3-(1-((7- methyl-5H-pyrrolo[2,3-b] pyrazin-2-yl)amino)ethyl) phenyl)-1H-pyrazole-4- carboxamide |
| 574 | 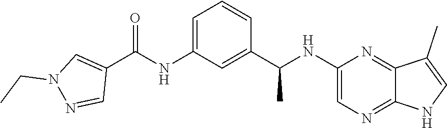 [SMILES: CCn1cc(C(=O)Nc2cccc([C@H](C)Nc3cnc4[nH]cc(C)c4n3)c2)cn1] | (S)-1-ethyl-N-(3-(1-((7-methyl- 5H-pyrrolo[2,3-b]pyrazin-2-yl) amino)ethyl)phenyl)-1H- pyrazole-4-carboxamide |
| 575 | 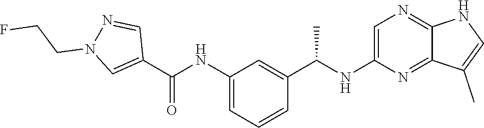 [SMILES: Cc1c[nH]c2ncc(N[C@@H](C)c3cccc(NC(=O)c4cnn(CCF)c4)c3)nc12] | (S)-1-(2-fluoroethyl)-N-(3-(1- ((7-methyl-5H-pyrrolo[2,3-b] pyrazin-2-yl)amino)ethyl) phenyl)-1H-pyrazole-4- carboxamide |
| 576 | 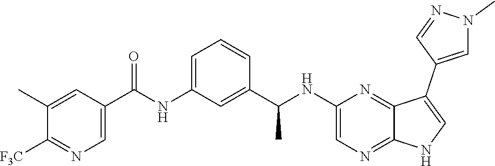 [SMILES: Cc1cc(C(=O)Nc2cccc([C@H](C)Nc3cnc4[nH]cc(-c5cnn(C)c5)c4n3)c2)cnc1C(F)(F)F] | (S)-5-methyl-N-(3-(1-((7-(1- methyl-1H-pyrazol-4-yl)-5H- pyrrolo[2,3-b]pyrazin-2-yl) amino)ethyl)phenyl)-6- (trifluoromethyl)nicotinamide |
| 577 | 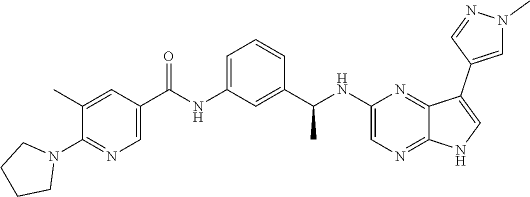 [SMILES: Cc1cc(C(=O)Nc2cccc([C@H](C)Nc3cnc4[nH]cc(-c5cnn(C)c5)c4n3)c2)cnc1N1CCCC1] | (S)-5-methyl-N-(3-(1-((7-(1- methyl-1H-pyrazol-4-yl)-5H- pyrrolo[2,3-b]pyrazin-2-yl) amino)ethyl)phenyl)-6- (pyrrolidin-1-yl)nicotinamide |
| 578 | 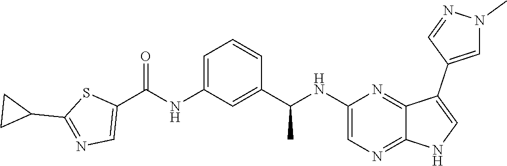 [SMILES: C[C@H](Nc1cnc2[nH]cc(-c3cnn(C)c3)c2n1)c1cccc(NC(=O)c2cnc(C3CC3)s2)c1] | (S)-2-cyclopropyl-N-(3-(1-((7- (1-methyl-1H-pyrazol-4-yl)-5H- pyrrolo[2,3-b]pyrazin-2-yl) amino)ethyl)phenyl)thiazole-5- carboxamide |
| 579 | 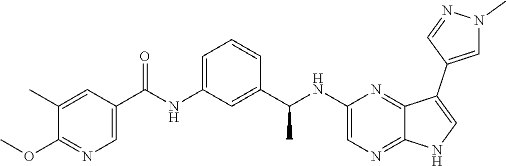 | (S)-6-methoxy-5-methyl-N-(3- (1-((7-(1-methyl-1H-pyrazol-4- yl)-5H-pyrrolo[2,3-b]pyrazin-2- yl)amino)ethyl)phenyl) nicotinamide |
| 580 | 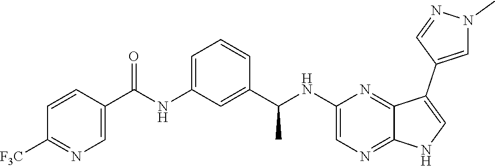 [SMILES: C[C@H](Nc1cnc2[nH]cc(-c3cnn(C)c3)c2n1)c1cccc(NC(=O)c2ccc(C(F)(F)F)nc2)c1] | (S)-N-(3-(1-((7-(1-methyl-1H- pyrazol-4-yl)-5H-pyrrolo[2,3-b] pyrazin-2-yl)amino)ethyl) phenyl)-6-(trifluoromethyl) nicotinamide |
| 581 | 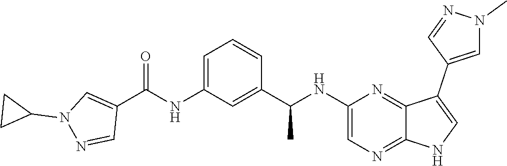 [SMILES: C[C@H](Nc1cnc2[nH]cc(-c3cnn(C)c3)c2n1)c1cccc(NC(=O)c2cnn(C3CC3)c2)c1] | (S)-1-cyclopropyl-N-(3-(1-((7- (1-methyl-1H-pyrazol-4-yl)-5H- pyrrolo[2,3-b]pyrazin-2-yl) amino)ethyl)phenyl)-1H- pyrazole-4-carboxamide |
| 582 | 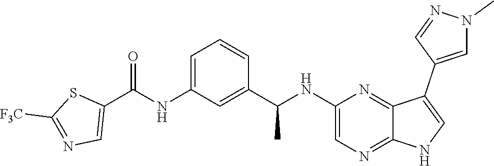 [SMILES: C[C@H](Nc1cnc2[nH]cc(-c3cnn(C)c3)c2n1)c1cccc(NC(=O)c2cnc(C(F)(F)F)s2)c1] | (S)-N-(3-(1-((7-(1-methyl-1H- pyrazol-4-yl)-5H-pyrrolo[2,3-b] pyrazin-2-yl)amino)ethyl) phenyl)-2-(trifluoromethyl) thiazole-5-carboxamide |
| 583 | 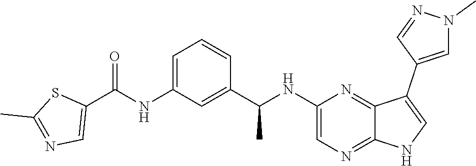 | (S)-2-methyl-N-(3-(1-((7-(1- methyl-1H-pyrazol-4-yl)-5H- pyrrolo[2,3-b]pyrazin-2-yl) amino)ethyl)phenyl)thiazole-5- carboxamide |
| 584 | 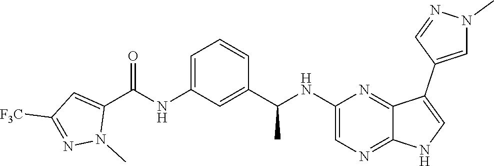 [SMILES: C[C@H](Nc1cnc2[nH]cc(-c3cnn(C)c3)c2n1)c1cccc(NC(=O)c2cc(C(F)(F)F)nn2C)c1] | (S)-1-methyl-N-(3-(1-((7-(1- methyl-1H-pyrazol-4-yl)-5H- pyrrolo[2,3-b]pyrazin-2-yl) amino)ethyl)phenyl)-3- (trifluoromethyl)-1H-pyrazole- 5-carboxamide |
| 585 | 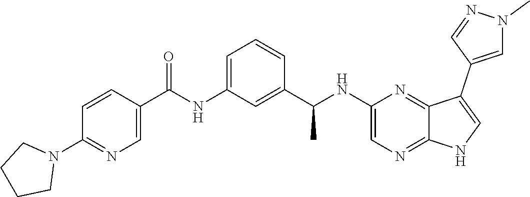 [SMILES: C[C@H](Nc1cnc2[nH]cc(-c3cnn(C)c3)c2n1)c1cccc(NC(=O)c2ccc(N3CCCC3)nc2)c1] | (S)-N-(3-(1-((7-(1-methyl-1H- pyrazol-4-yl)-5H-pyrrolo[2,3-b] pyrazin-2-yl)amino)ethyl) phenyl)-6-(pyrrolidin-1-yl) nicotinamide |
| 586 | 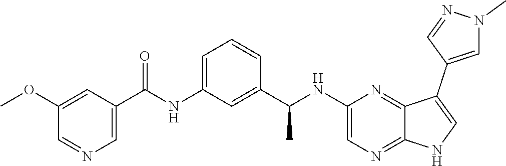 | (S)-5-methoxy-N-(3-(1-((7-(1- methyl-1H-pyrazol-4-yl)-5H- pyrrolo[2,3-b]pyrazin-2-yl) amino)ethyl)phenyl) nicotinamide |
| 587 | 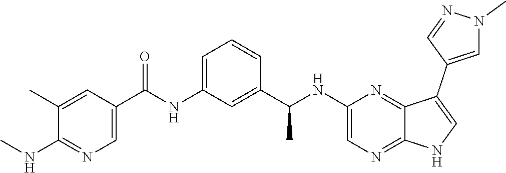 | (S)-5-methyl-N-(3-(1-((7-(1- methyl-1H-pyrazol-4-yl)-5H- pyrrolo[2,3-b]pyrazin-2-yl) amino)ethyl)phenyl)-6- (methylamino)nicotinamide |
| 588 | 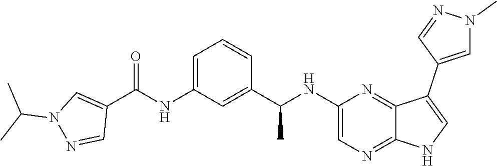 [SMILES: CC(C)n1cc(C(=O)Nc2cccc([C@H](C)Nc3cnc4[nH]cc(-c5cnn(C)c5)c4n3)c2)cn1] | (S)-1-isopropyl-N-(3-(1-((7-(1- methyl-1H-pyrazol-4-yl)-5H- pyrrolo[2,3-b]pyrazin-2-yl) amino)ethyl)phenyl)-1H- pyrazole-4-carboxamide |
| 589 | 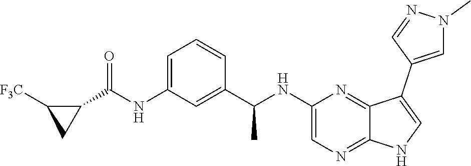 [SMILES: C[C@H](Nc1cnc2[nH]cc(-c3cnn(C)c3)c2n1)c1cccc(NC(=O)[C@@H]2C[C@H]2C(F)(F)F)c1] | (1R,2R)-N-(3-((S)-1-((7-(1- methyl-1H-pyrazol-4-yl)-5H- pyrrolo[2,3-b]pyrazin-2-yl) amino)ethyl)phenyl)-2- (trifluoromethyl)cyclopropane- 1-carboxamide |
| 590 | 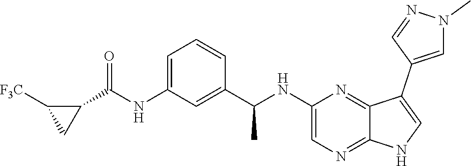 [SMILES: C[C@H](Nc1cnc2[nH]cc(-c3cnn(C)c3)c2n1)c1cccc(NC(=O)[C@@H]2C[C@@H]2C(F)(F)F)c1] | (1R,2S)-N-(3-((5)-1-((7-(1- methyl-1H-pyrazol-4-yl)-5H- pyrrolo[2,3-b]pyrazin-2-yl) amino)ethyl)phenyl)-2- (trifluoromethyl)cyclopropane- 1-carboxamide |
| 591 | 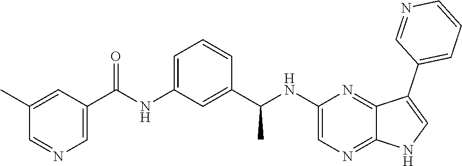 [SMILES: Cc1cncc(C(=O)Nc2cccc([C@H](C)Nc3cnc4[nH]cc(-c5cccnc5)c4n3)c2)c1] | (S)-5-methyl-N-(3-(1-((7- (pyridin-3-yl)-5H-pyrrolo[2,3-b] pyrazin-2-yl)amino)ethyl) phenyl)nicotinamide |
| 592 | 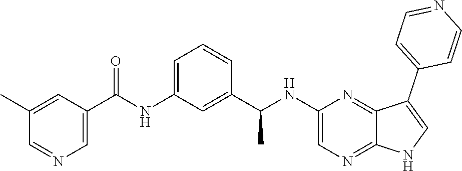 [SMILES: Cc1cncc(C(=O)Nc2cccc([C@H](C)Nc3cnc4[nH]cc(-c5ccncc5)c4n3)c2)c1] | (S)-5-methyl-N-(3-(1-((7- (pyridin-4-yl)-5H-pyrrolo[2,3-b] pyrazin-2-yl)amino)ethyl) phenyl)nicotinamide |
| 593 | 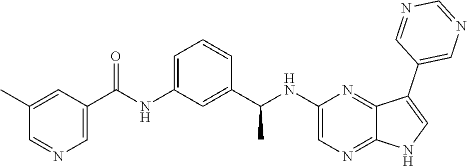 [SMILES: Cc1cncc(C(=O)Nc2cccc([C@H](C)Nc3cnc4[nH]cc(-c5cncnc5)c4n3)c2)c1] | (S)-5-methyl-N-(3-(1-((7- (pyrimidin-5-yl)-5H-pyrrolo [2,3-b]pyrazin-2-yl)amino) ethyl)phenyl)nicotinamide |
| 594 | 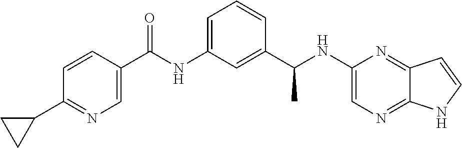 [SMILES: C[C@H](Nc1cnc2[nH]ccc2n1)c1cccc(NC(=O)c2ccc(C3CC3)nc2)c1] | (S)-N-(3-(1-((5H-pyrrolo[2,3-b] pyrazin-2-yl)amino)ethyl) phenyl)-6- cyclopropylnicotinamide |
| 595 | 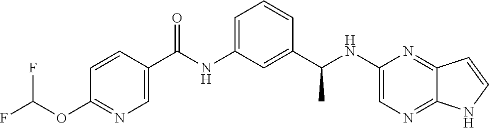 [SMILES: C[C@H](Nc1cnc2[nH]ccc2n1)c1cccc(NC(=O)c2ccc(OC(F)F)nc2)c1] | (S)-N-(3-(1-((5H-pyrrolo[2,3-b] pyrazin-2-yl)amino)ethyl) phenyl)-6- (difluoromethoxy)nicotinamide |
| 596 | 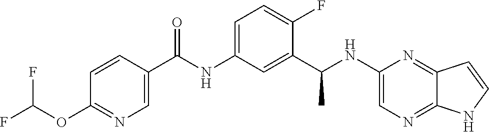 [SMILES: C[C@H](Nc1cnc2[nH]ccc2n1)c1cc(NC(=O)c2ccc(OC(F)F)nc2)ccc1F] | (S)-N-(3-(1-((5H-pyrrolo[2,3-b] pyrazin-2-yl)amino)ethyl)-4- fluorophenyl)-6- (difluoromethoxy)nicotinamide |
| 597 | 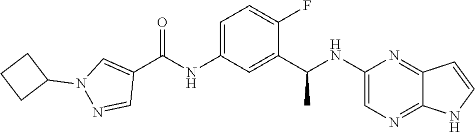 [SMILES: C[C@H](Nc1cnc2[nH]ccc2n1)c1cc(NC(=O)c2cnn(C3CCC3)c2)ccc1F] | (S)-N-(3-(1-((5H-pyrrolo[2,3-b] pyrazin-2-yl)amino)ethyl)-4- fluorophenyl)-1-cyclobutyl-1H- pyrazole-4-carboxamide |
| 598 | 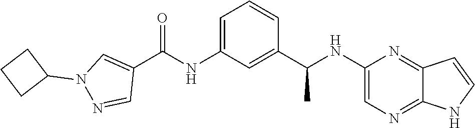 [SMILES: C[C@H](Nc1cnc2[nH]ccc2n1)c1cccc(NC(=O)c2cnn(C3CCC3)c2)c1] | (S)-N-(3-(1-((5H-pyrrolo[2,3-b] pyrazin-2-yl)amino)ethyl) phenyl)-1-cyclobutyl-1H- pyrazole-4-carboxamide |
| 599 | 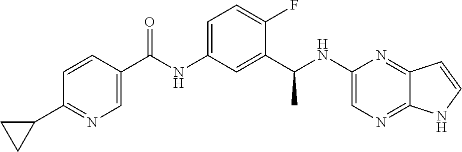 [SMILES: C[C@H](Nc1cnc2[nH]ccc2n1)c1cc(NC(=O)c2ccc(C3CC3)nc2)ccc1F] | (S)-N-(3-(1-((5H-pyrrolo[2,3-b] pyrazin-2-yl)amino)ethyl)-4- fluorophenyl)-6- cyclopropylnicotinamide |
| 600 | 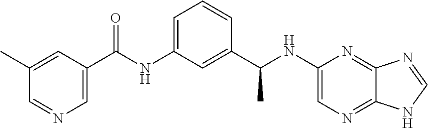 [SMILES: Cc1cncc(C(=O)Nc2cccc([C@H](C)Nc3cnc4[nH]cnc4n3)c2)c1] | (S)-N-(3-(1-((3H-imidazo[4,5-b] pyridin-6-yl)amino)ethyl) phenyl)-5-methylnicotinamide |
| 601 | 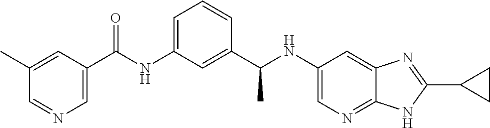 [SMILES: Cc1cncc(C(=O)Nc2cccc([C@H](C)Nc3cnc4[nH]c(C5CC5)nc4c3)c2)c1] | (S)-N-(3-(1-((2-cyclopropyl-3H- imidazo[4,5-6]pyridin-6-yl) amino)ethyl)phenyl)-5- methylnicotinamide |
| 602 | 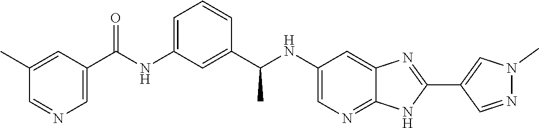 | (S)-5-methyl-N-(3-(1-((2-(1- methyl-1H-pyrazol-4-yl)-3H- imidazo[4,5-b]pyridin-6-yl) amino)ethyl)phenyl) nicotinamide |
| 603 | 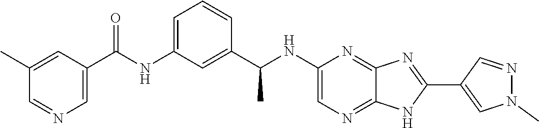 | (S)-5-methyl-N-(3-(1-((2-(1- methyl-1H-pyrazol-4-yl)-1H- imidazo[4,5-b]pyrazin-5-yl ) amino)ethyl)phenyl) nicotinamide |
| 604 | 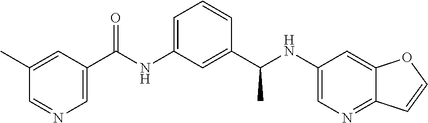 [SMILES: Cc1cncc(C(=O)Nc2cccc([C@H](C)Nc3cnc4ccoc4c3)c2)c1] | (S)-N-(3-(1-(furo[3,2-b]pyridin- 6-ylamino)ethyl)phenyl)-5- methylnicotinamide |
| 605 | 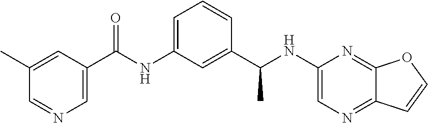 [SMILES: Cc1cncc(C(=O)Nc2cccc([C@H](C)Nc3cnc4ccoc4n3)c2)c1] | (S)-N-(3-(1-(furo[2,3-b]pyrazin- 3-ylamino)ethyl)phenyl)-5- methylnicotinamide |
| 606 | 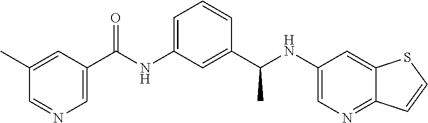 [SMILES: Cc1cncc(C(=O)Nc2cccc([C@H](C)Nc3cnc4ccsc4c3)c2)c1] | (S)-5-methyl-N-(3-(1- (thieno[3,2-b]pyridin-6- ylamino)ethyl)phenyl) nicotinamide |
| 607 | 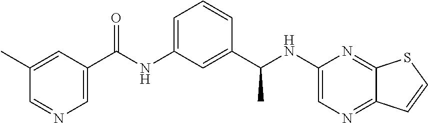 [SMILES: Cc1cncc(C(=O)Nc2cccc([C@H](C)Nc3cnc4ccsc4n3)c2)c1] | (S)-5-methyl-N-(3-(1- (thieno[2,3-b]pyridin-3- ylamino)ethyl)phenyl) nicotinamide |
| 608 | 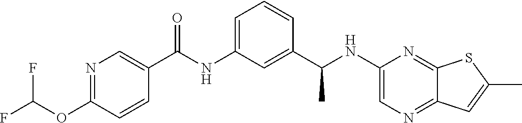 [SMILES: Cc1cc2ncc(N[C@@H](C)c3cccc(NC(=O)c4ccc(OC(F)F)nc4)c3)nc2s1] | (S)-6-(difluoromethoxy)-N-(3- (1-((6-methylthieno[2,3-b] pyrazin-3-yl)amino)ethyl) phenyl)nicotinamide |
| 609 | 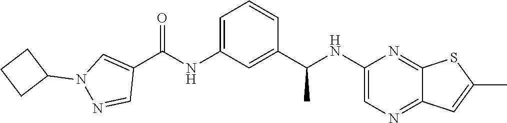 [SMILES: Cc1cc2ncc(N[C@@H](C)c3cccc(NC(=O)c4cnn(C5CCC5)c4)c3)nc2s1] | (S)-1-cyclobutyl-N-(3-(1-((6- methylthieno[2,3-b]pyrazin-3- yl)amino)ethyl)phenyl)-1H- pyrazole-4-carboxamide |
| 610 | 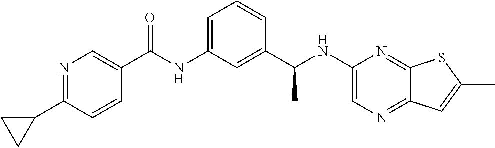 [SMILES: Cc1cc2ncc(N[C@@H](C)c3cccc(NC(=O)c4ccc(C5CC5)nc4)c3)nc2s1] | (S)-6-cyclopropyl-N-(3-(1-((6- methylthieno[2,3-b]pyrazin-3- yl)amino)ethyl)phenyl) nicotinamide |
| 611 | 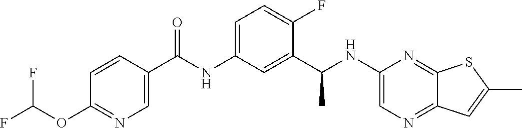 [SMILES: Cc1cc2ncc(N[C@@H](C)c3cc(NC(=O)c4ccc(OC(F)F)nc4)ccc3F)nc2s1] | (S)-6-(difluoromethoxy)-N-(4- fluoro-3-(1-((6- methylthieno[2,3-b]pyrazin-3- yl)amino)ethyl)phenyl) nicotinamide |
| 612 | 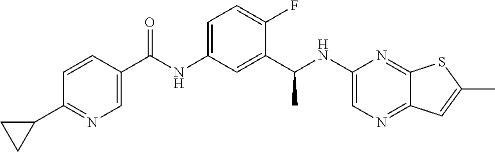 [SMILES: Cc1cc2ncc(N[C@@H](C)c3cc(NC(=O)c4ccc(C5CC5)nc4)ccc3F)nc2s1] | (S)-6-cyclopropyl-N-(4-fluoro- 3-(1-((6-methylthieno[2,3-b] pyrazin-3-yl)amino)ethyl) phenyl)nicotinamide |
| 613 | 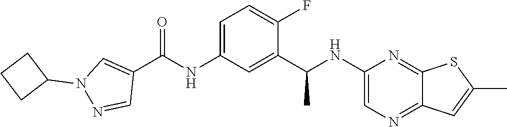 [SMILES: Cc1cc2ncc(N[C@@H](C)c3cc(NC(=O)c4cnn(C5CCC5)c4)ccc3F)nc2s1] | (S)-1-cyclobutyl-N-(4-fluoro-3- (1-((6-methylthieno[2,3-b] pyrazin-3-yl)amino)ethyl) phenyl)-1H-pyrazole-4- carboxamide |
| 614 | 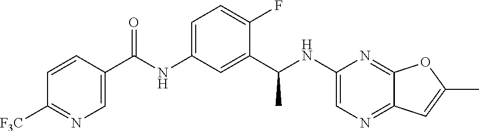 [SMILES: Cc1cc2ncc(N[C@@H](C)c3cc(NC(=O)c4ccc(C(F)(F)F)nc4)ccc3F)nc2o1] | (S)-N-(4-fluoro-3-(1-((6- methylfuro[2,3-b]pyrazin-3-yl) amino)ethyl)phenyl)-6- (trifluoromethyl)nicotinamide |
| 615 | 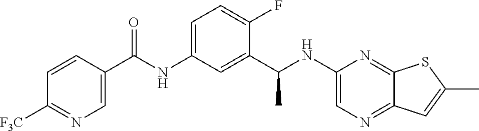 [SMILES: Cc1cc2ncc(N[C@@H](C)c3cc(NC(=O)c4ccc(C(F)(F)F)nc4)ccc3F)nc2s1] | (S)-N-(4-fluoro-3-(1-((6- methylthieno[2,3-b]pyrazin-3- yl)amino)ethyl)phenyl)-6- (trifluoromethyl)nicotinamide |
| 616 | 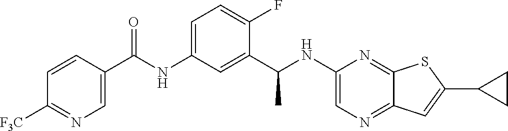 [SMILES: C[C@H](Nc1cnc2cc(C3CC3)sc2n1)c1cc(NC(=O)c2ccc(C(F)(F)F)nc2)ccc1F] | (S)-N-(3-(1-((6- cyclopropylthieno[2,3-b] pyrazin-3-yl)amino)ethyl)-4- fluorophenyl)-6- (trifluoromethyl)nicotinamide |
| 617 | 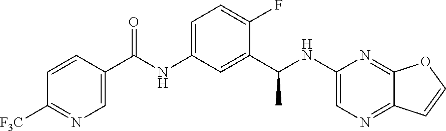 [SMILES: C[C@H](Nc1cnc2ccoc2n1)c1cc(NC(=O)c2ccc(C(F)(F)F)nc2)ccc1F] | (S)-N-(4-fluoro-3-(1-(furo[2,3- b]pyrazin-3-ylamino)ethyl) phenyl)-6-(trifluoromethyl) nicotinamide |
| 618 | 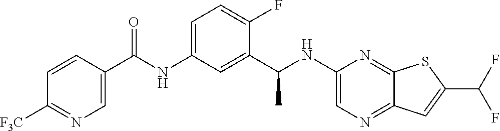 [SMILES: C[C@H](Nc1cnc2cc(C(F)F)sc2n1)c1cc(NC(=O)c2ccc(C(F)(F)F)nc2)ccc1F] | (S)-N-(3-(1-((6-(difluoromethyl) thieno[2,3-b]pyrazin-3-yl) amino)ethyl)-4-fluorophenyl)-6- (trifluoromethyl)nicotinamide |
| 619 | 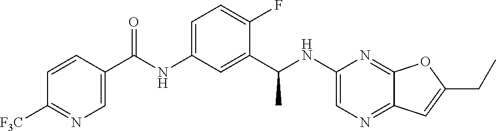 [SMILES: CCc1cc2ncc(N[C@@H](C)c3cc(NC(=O)c4ccc(C(F)(F)F)nc4)ccc3F)nc2o1] | (S)-N-(3-(1-((6-ethylfuro[2,3-b] pyrazin-3-yl)amino)ethyl)-4- fluorophenyl)-6- (trifluoromethyl)nicotinamide |
| 620 | 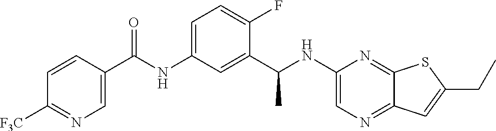 [SMILES: CCc1cc2ncc(N[C@@H](C)c3cc(NC(=O)c4ccc(C(F)(F)F)nc4)ccc3F)nc2s1] | (S)-N-(3-(1-((6-ethylthieno[2,3- b]pyrazin-3-yl)amino)ethyl)-4- fluorophenyl)-6- (trifluoromethyl)nicotinamide |
| 621 | 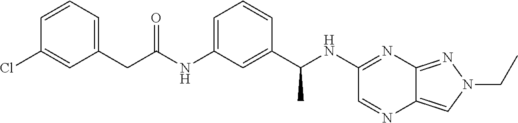 [SMILES: CCn1cc2ncc(N[C@@H](C)c3cccc(NC(=O)Cc4cccc(Cl)c4)c3)nc2n1] | (S)-2-(3-chlorophenyl)-N-(3-(1- ((2-ethyl-2H-pyrazolo[3,4-b] pyrazin-6-yl)amino)ethyl) phenyl)acetamide |
| 622 | 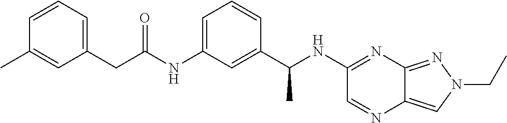 [SMILES: CCn1cc2ncc(N[C@@H](C)c3cccc(NC(=O)Cc4cccc(C)c4)c3)nc2n1] | (S)-N-(3-(1-((2-ethyl-2H- pyrazolo[3,4-b]pyrazin-6-yl) amino)ethyl)phenyl)-2-(m-tolyl) acetamide |
| 623 | 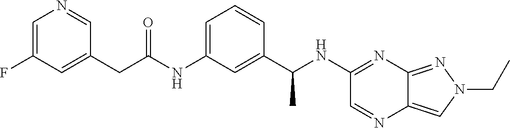 [SMILES: CCn1cc2ncc(N[C@@H](C)c3cccc(NC(=O)Cc4cncc(F)c4)c3)nc2n1] | (S)-N-(3-(1-((2-ethyl-2H- pyrazolo[3,4-b]pyrazin-6-yl) amino)ethyl)phenyl)-2-(5- fluoropyridin-3-yl)acetamide |
| 624 | 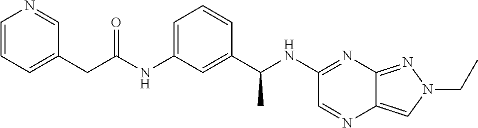 [SMILES: CCn1cc2ncc(N[C@@H](C)c3cccc(NC(=O)Cc4cccnc4)c3)nc2n1] | (S)-N-(3-(1-((2-ethyl-2H- pyrazolo[3,4-b]pyrazin-6-yl) amino)ethyl)phenyl)-2-(pyridin- 3-yl)acetamide |
| 625 | 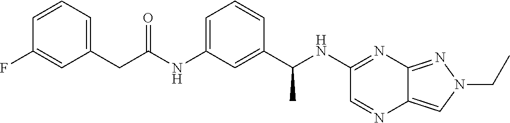 [SMILES: CCn1cc2ncc(N[C@@H](C)c3cccc(NC(=O)Cc4cccc(F)c4)c3)nc2n1] | (S)-N-(3-(1-((2-ethyl-2H- pyrazolo[3,4-b]pyrazin-6-yl) amino)ethyl)phenyl)-2-(3- fluorophenyl)acetamide |
| 626 | 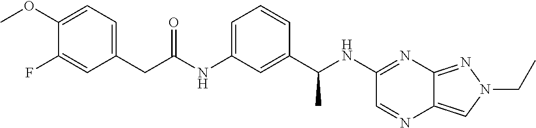 [SMILES: CCn1cc2ncc(N[C@@H](C)c3cccc(NC(=O)Cc4ccc(OC)c(F)c4)c3)nc2n1] | (S)-N-(3-(1-((2-ethyl-2H- pyrazolo[3,4-b]pyrazin-6-yl) amino)ethyl)phenyl)-2-(3- fluoro-4-methoxyphenyl) acetamide |
| 627 | 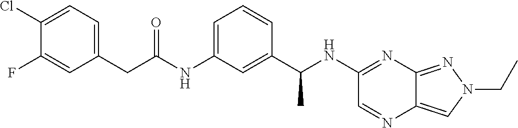 [SMILES: CCn1cc2ncc(N[C@@H](C)c3cccc(NC(=O)Cc4ccc(Cl)c(F)c4)c3)nc2n1] | (S)-2-(4-chloro-3-fluorophenyl)- N-(3-(1-((2-ethyl-2H- pyrazolo[3,4-b]pyrazin-6-yl) amino)ethyl)phenyl)acetamide |
| 628 | 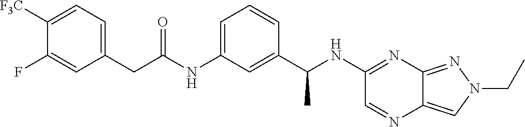 [SMILES: CCn1cc2ncc(N[C@@H](C)c3cccc(NC(=O)Cc4ccc(C(F)(F)F)c(F)c4)c3)nc2n1] | (S)-N-(3-(1-((2-ethyl-2H- pyrazolo[3,4-b]pyrazin-6-yl) amino)ethyl)phenyl)-2-(3- fluoro-4-(trifluoromethyl) phenyl)acetamide |
| 629 | 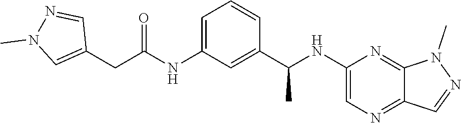 [SMILES: C[C@H](Nc1cnc2cnn(C)c2n1)c1cccc(NC(=O)Cc2cnn(C)c2)c1] | (S)-2-(1-methyl-1H-pyrazol-4- yl)-N-(3-(1-((1-methyl-1H- pyrazolo[3,4-b]pyrazin-6-yl) amino)ethyl)phenyl)acetamide |
| 630 | 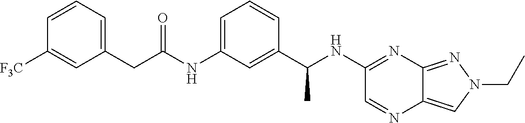 [SMILES: CCn1cc2ncc(N[C@@H](C)c3cccc(NC(=O)Cc4cccc(C(F)(F)F)c4)c3)nc2n1] | (S)-N-(3-(1-((2-ethyl-2H- pyrazolo[3,4-b]pyrazin-6-yl) amino)ethyl)phenyl)-2-(3- (trifluoromethyl)phenyl) acetamide |
| 631 | 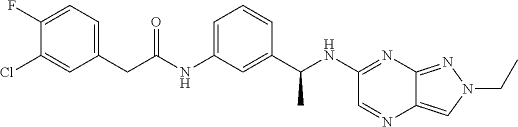 [SMILES: CCn1cc2ncc(N[C@@H](C)c3cccc(NC(=O)Cc4ccc(F)c(Cl)c4)c3)nc2n1] | (S)-2-(3-chloro-4-fluorophenyl)- N-(3-(1-((2-ethyl-2H- pyrazolo[3,4-b]pyrazin-6-yl) amino)ethyl)phenyl)acetamide |
| 632 | 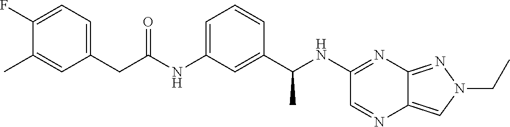 [SMILES: CCn1cc2ncc(N[C@@H](C)c3cccc(NC(=O)Cc4ccc(F)c(C)c4)c3)nc2n1] | (S)-N-(3-(1-((2-ethyl-2H- pyrazolo[3,4-b]pyrazin-6-yl) amino)ethyl)phenyl)-2-(4- fluoro-3-methylphenyl) acetamide |
| 633 | 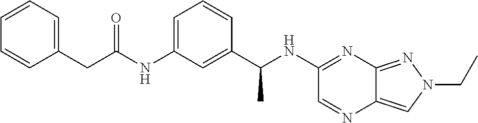 [SMILES: CCn1cc2ncc(N[C@@H](C)c3cccc(NC(=O)Cc4ccccc4)c3)nc2n1] | (S)-N-(3-(1-((2-ethyl-2H- pyrazolo[3,4-b]pyrazin-6-yl) amino)ethyl)phenyl)-2- phenylacetamide |
| 634 | 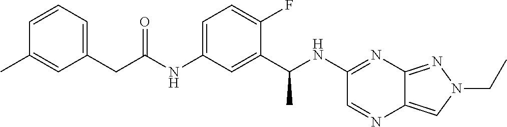 [SMILES: CCn1cc2ncc(N[C@@H](C)c3cc(NC(=O)Cc4cccc(C)c4)ccc3F)nc2n1] | (S)-N-(3-(1-((2-ethyl-2H- pyrazolo[3,4-b]pyrazin-6-yl) amino)ethyl)-4-fluorophenyl)-2- (m-tolyl)acetamide |
| 635 | 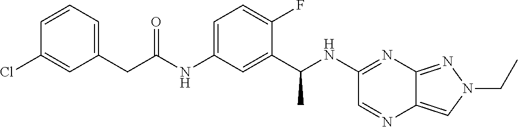 [SMILES: CCn1cc2ncc(N[C@@H](C)c3cc(NC(=O)Cc4cccc(Cl)c4)ccc3F)nc2n1] | (S)-2-(3-chlorophenyl)-N-(3-(1- ((2-ethyl-2H-pyrazolo[3,4-b] pyrazin-6-yl)amino)ethyl)-4- fluorophenyl)acetamide |
| 636 | 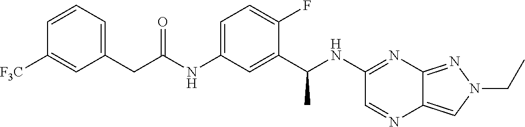 [SMILES: CCn1cc2ncc(N[C@@H](C)c3cc(NC(=O)Cc4cccc(C(F)(F)F)c4)ccc3F)nc2n1] | (S)-N-(3-(1-((2-ethyl-2H- pyrazolo[3,4-b]pyrazin-6-yl) amino)ethyl)-4-fluorophenyl)-2- (3-(trifluoromethyl)phenyl) acetamide |
| 637 | 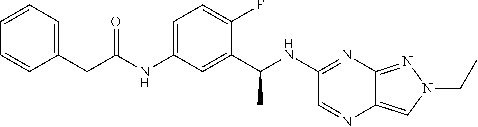 [SMILES: CCn1cc2ncc(N[C@@H](C)c3cc(NC(=O)Cc4ccccc4)ccc3F)nc2n1] | (S)-N-(3-(1-((2-ethyl-2H- pyrazolo[3,4-b]pyrazin-6-yl) amino)ethyl)-4-fluorophenyl)-2- phenylacetamide |
| 638 | 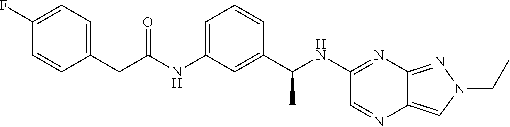 [SMILES: CCn1cc2ncc(N[C@@H](C)c3cccc(NC(=O)Cc4ccc(F)cc4)c3)nc2n1] | (S)-N-(3-(1-((2-ethyl-2H- pyrazolo[3,4-b]pyrazin-6-yl) amino)ethyl)phenyl)-2-(4- fluorophenyl)acetamide |
| 639 | 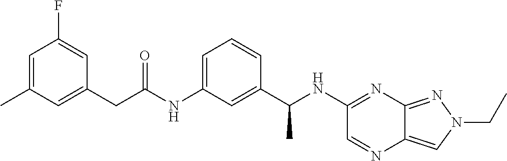 [SMILES: CCn1cc2ncc(N[C@@H](C)c3cccc(NC(=O)Cc4cc(C)cc(F)c4)c3)nc2n1] | (S)-N-(3-(1-((2-ethyl-2H- pyrazolo[3,4-b]pyrazin-6-yl) amino)ethyl)phenyl)-2-(3- fluoro-5-methylphenyl) acetamide |
| 640 | 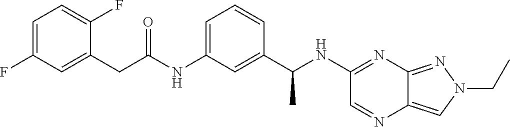 [SMILES: CCn1cc2ncc(N[C@@H](C)c3cccc(NC(=O)Cc4cc(F)ccc4F)c3)nc2n1] | (S)-2-(2,5-difluorophenyl)-N-(3- (1-((2-ethyl-2H-pyrazolo[3,4-b] pyrazin-6-yl)amino)ethyl) phenyl)acetamide |
| 641 | 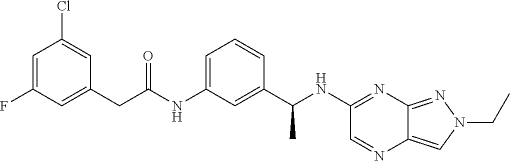 [SMILES: CCn1cc2ncc(N[C@@H](C)c3cccc(NC(=O)Cc4cc(F)cc(Cl)c4)c3)nc2n1] | (S)-2-(3-chloro-5-fluorophenyl)- N-(3-(1-((2-ethyl-2H- pyrazolo[3,4-b]pyrazin-6-yl) amino)ethyl)phenyl)acetamide |
| 642 | 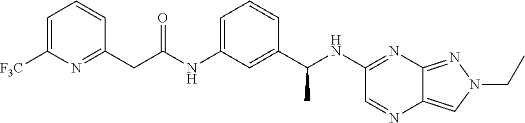 [SMILES: CCn1cc2ncc(N[C@@H](C)c3cccc(NC(=O)Cc4cccc(C(F)(F)F)n4)c3)nc2n1] | (S)-N-(3-(1-((2-ethyl-2H- pyrazolo[3,4-b]pyrazin-6-yl) amino)ethyl)phenyl)-2-(6- (trifluoromethyl)pyridin-2-yl) acetamide |
| 643 | 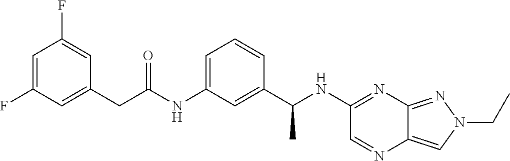 [SMILES: CCn1cc2ncc(N[C@@H](C)c3cccc(NC(=O)Cc4cc(F)cc(F)c4)c3)nc2n1] | (S)-2-(3,5-difluorophenyl)-N-(3- (1-((2-ethyl-2H-pyrazolo[3,4-b] pyrazin-6-yl)amino)ethyl) phenyl)acetamide |
| 644 | 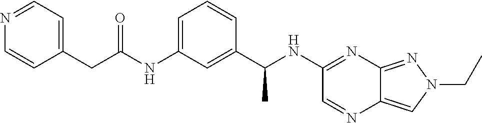 [SMILES: CCn1cc2ncc(N[C@@H](C)c3cccc(NC(=O)Cc4ccncc4)c3)nc2n1] | (S)-N-(3-(1-((2-ethyl-2H- pyrazolo[3,4-b]pyrazin-6-yl) amino)ethyl)phenyl)-2-(pyridin- 4-yl)acetamide |
| 645 | 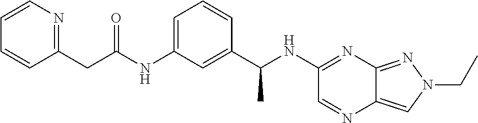 [SMILES: CCn1cc2ncc(N[C@@H](C)c3cccc(NC(=O)Cc4ccccn4)c3)nc2n1] | (S)-N-(3-(1-((2-ethyl-2H- pyrazolo[3,4-b]pyrazin-6-yl) amino)ethyl)phenyl)-2-(pyridin- 2-yl)acetamide |
| 646 | 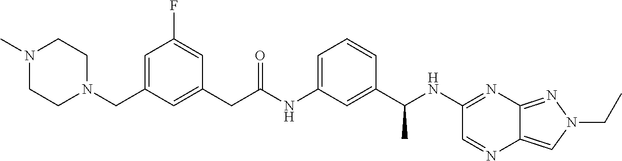 [SMILES: CCn1cc2ncc(N[C@@H](C)c3cccc(NC(=O)Cc4cc(F)cc(CN5CCN(C)CC5)c4)c3)nc2n1] | (S)-N-(3-(1-((2-ethyl-2H- pyrazolo[3,4-b]pyrazin-6-yl) amino)ethyl)phenyl)-2-(3- fluoro-5-((4-methylpiperazin-1- yl)methyl)phenyl)acetamide |
| 647 | 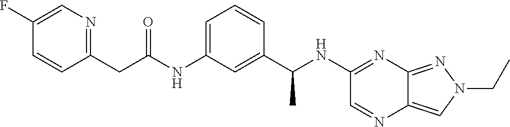 [SMILES: CCn1cc2ncc(N[C@@H](C)c3cccc(NC(=O)Cc4ccc(F)cn4)c3)nc2n1] | (S)-N-(3-(1-((2-ethyl-2H- pyrazolo[3,4-b]pyrazin-6-yl) amino)ethyl)phenyl)-2-(5- fluoropyridin-2-yl)acetamide |
| 648 | 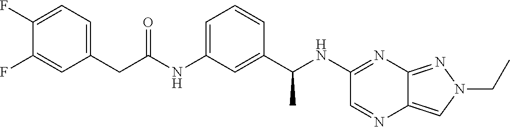 [SMILES: CCn1cc2ncc(N[C@@H](C)c3cccc(NC(=O)Cc4ccc(F)c(F)c4)c3)nc2n1] | (S)-2-(3,4-difluorophenyl)-N-(3- (1-((2-ethyl-2H-pyrazolo[3,4-b] pyrazin-6-yl)amino)ethyl) phenyl)acetamide |
| 649 | 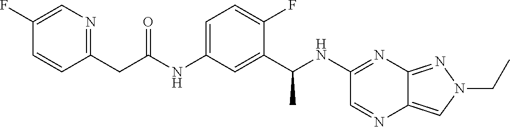 [SMILES: CCn1cc2ncc(N[C@@H](C)c3cc(NC(=O)Cc4ccc(F)cn4)ccc3F)nc2n1] | (S)-N-(3-(1-((2-ethyl-2H- pyrazolo[3,4-b]pyrazin-6-yl) amino)ethyl)-4-fluorophenyl)-2- (5-fluoropyridin-2-yl)acetamide |
| 650 | 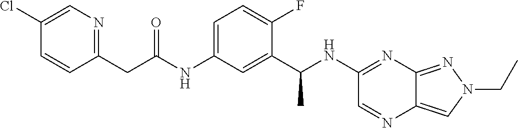 [SMILES: CCn1cc2ncc(N[C@@H](C)c3cc(NC(=O)Cc4ccc(Cl)cn4)ccc3F)nc2n1] | (S)-2-(5-chloropyridin-2-yl)-N- (3-(1-((2-ethyl-2H-pyrazolo[3,4- b]pyrazin-6-yl)amino)ethyl)-4- fluorophenyl)acetamide |
| 651 | 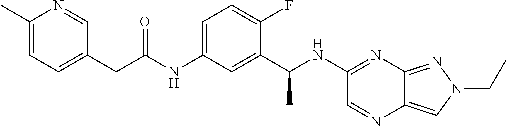 [SMILES: CCn1cc2ncc(N[C@@H](C)c3cc(NC(=O)Cc4ccc(C)nc4)ccc3F)nc2n1] | (S)-N-(3-(1-((2-ethyl-2H- pyrazolo[3,4-b]pyrazin-6-yl) amino)ethyl)-4-fluorophenyl)-2- (6-methylpyridin-3-yl) acetamide |
| 652 | 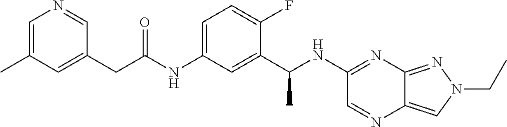 [SMILES: CCn1cc2ncc(N[C@@H](C)c3cc(NC(=O)Cc4cncc(C)c4)ccc3F)nc2n1] | (S)-N-(3-(1-((2-ethyl-2H- pyrazolo[3,4-b]pyrazin-6-yl) amino)ethyl)-4-fluorophenyl)-2- (5-methylpyridin-3-yl) acetamide |
| 653 | 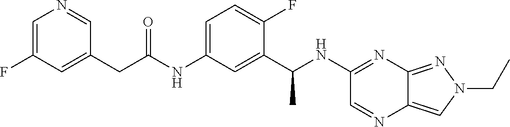 [SMILES: CCn1cc2ncc(N[C@@H](C)c3cc(NC(=O)Cc4cncc(F)c4)ccc3F)nc2n1] | (S)-N-(3-(1-((2-ethyl-2H- pyrazolo[3,4-b]pyrazin-6-yl) amino)ethyl)-4-fluorophenyl)-2- (5-fluoropyridin-3-yl)acetamide |
| 654 | 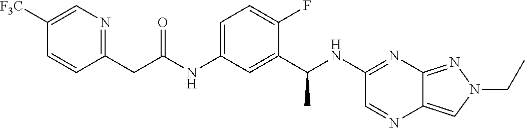 [SMILES: CCn1cc2ncc(N[C@@H](C)c3cc(NC(=O)Cc4ccc(C(F)(F)F)cn4)ccc3F)nc2n1] | (S)-N-(3-(1-((2-ethyl-2H- pyrazolo[3,4-b]pyrazin-6-yl) amino)ethyl)-4-fluorophenyl)-2- (5-(trifluoromethyl)pyridin-2-yl) acetamide |
| 655 | 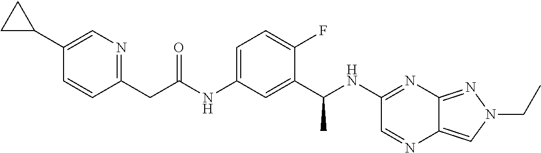 [SMILES: CCn1cc2ncc(N[C@@H](C)c3cc(NC(=O)Cc4ccc(C5CC5)cn4)ccc3F)nc2n1] | (S)-2-(5-cyclopropylpyridin-2- yl)-N-(3-(1-((2-ethyl-2H- pyrazolo[3,4-b]pyrazin-6-yl) amino)ethyl)-4-fluorophenyl) acetamide |
| 656 | 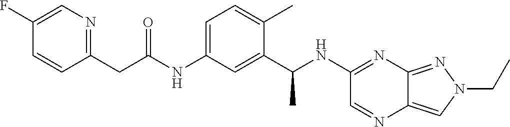 [SMILES: CCn1cc2ncc(N[C@@H](C)c3cc(NC(=O)Cc4ccc(F)cn4)ccc3C)nc2n1] | (S)-N-(3-(1-((2-ethyl-2H- pyrazolo[3,4-b]pyrazin-6-yl) amino)ethyl)-4-methylphenyl)- 2-(5-fluoropyridin-2-yl) acetamide |
| 657 | 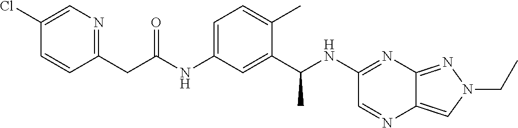 [SMILES: CCn1cc2ncc(N[C@@H](C)c3cc(NC(=O)Cc4ccc(Cl)cn4)ccc3C)nc2n1] | (S)-2-(5-chloropyridin-2-yl)-N- (3-(1-((2-ethyl-2H-pyrazolo[3,4- b]pyrazin-6-yl)amino)ethyl)-4- methylphenyl)acetamide |
| 658 | 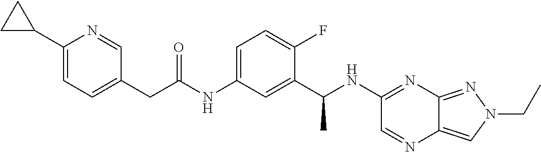 [SMILES: CCn1cc2ncc(N[C@@H](C)c3cc(NC(=O)Cc4ccc(C5CC5)nc4)ccc3F)nc2n1] | (S)-2-(6-cyclopropylpyridin-3 - yl)-N-(3-(1-((2-ethyl-2H- pyrazolo[3,4-b]pyrazin-6-yl) amino)ethyl)-4-fluorophenyl) acetamide |
| 659 | 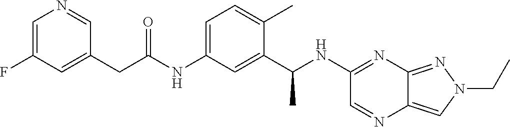 [SMILES: CCn1cc2ncc(N[C@@H](C)c3cc(NC(=O)Cc4cncc(F)c4)ccc3C)nc2n1] | (S)-N-(3-(1-((2-ethyl-2H- pyrazolo[3,4-b]pyrazin-6-yl) amino)ethyl)-4-methylphenyl)- 2-(5-fluoropyridin-3-yl) acetamide |
| 660 | 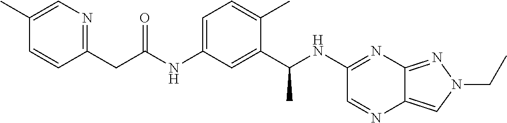 [SMILES: CCn1cc2ncc(N[C@@H](C)c3cc(NC(=O)Cc4ccc(C)cn4)ccc3C)nc2n1] | (S)-N-(3-(1-((2-ethyl-2H- pyrazolo[3,4-b]pyrazin-6-yl) amino)ethyl)-4-methylphenyl)- 2-(5-methylpyridin-2-yl) acetamide |
| 661 | 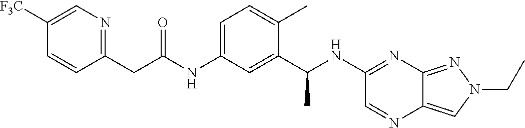 [SMILES: CCn1cc2ncc(N[C@@H](C)c3cc(NC(=O)Cc4ccc(C(F)(F)F)cn4)ccc3C)nc2n1] | (S)-N-(3-(1-((2-ethyl-2H- pyrazolo[3,4-b]pyrazin-6-yl) amino)ethyl)-4-methylphenyl)- 2-(5-(trifluoromethyl)pyridin-2- yl)acetamide |
| 662 | 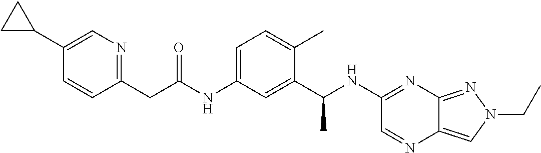 [SMILES: CCn1cc2ncc(N[C@@H](C)c3cc(NC(=O)Cc4ccc(C5CC5)cn4)ccc3C)nc2n1] | (S)-2-(5-cyclopropylpyridin-2- yl)-N-(3-(1-((2-ethyl-2H- pyrazolo[3,4-b]pyrazin-6-yl) amino)ethyl)-4-methylphenyl) acetamide |
| 663 | 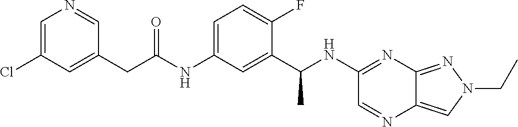 [SMILES: CCn1cc2ncc(N[C@@H](C)c3cc(NC(=O)Cc4cncc(Cl)c4)ccc3F)nc2n1] | (S)-2-(5-chloropyridin-3-yl)-N- (3-(1-((2-ethyl-2H-pyrazolo[3,4- b]pyrazin-6-yl)amino)ethyl)-4- fluorophenyl)acetamide |
| 664 | 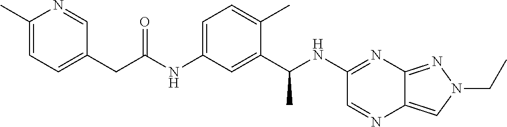 [SMILES: CCn1cc2ncc(N[C@@H](C)c3cc(NC(=O)Cc4ccc(C)nc4)ccc3C)nc2n1] | (S)-N-(3-(1-((2-ethyl-2H- pyrazolo[3,4-b]pyrazin-6-yl) amino)ethyl)-4-methylphenyl)- 2-(6-methylpyridin-3-yl) acetamide |
| 665 | 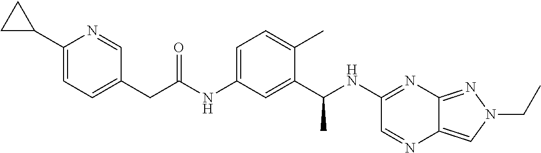 [SMILES: CCn1cc2ncc(N[C@@H](C)c3cc(NC(=O)Cc4ccc(C5CC5)nc4)ccc3C)nc2n1] | (S)-2-(6-cyclopropylpyridin-3- yl)-N-(3-(1-((2-ethyl-2H- pyrazolo[3,4-b]pyrazin-6-yl) amino)ethyl)-4-methylphenyl) acetamide |
| 666 | 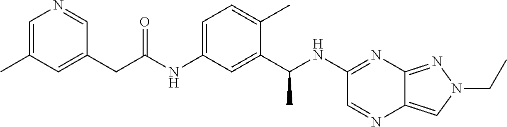 [SMILES: CCn1cc2ncc(N[C@@H](C)c3cc(NC(=O)Cc4cncc(C)c4)ccc3C)nc2n1] | (S)-N-(3-(1-((2-ethyl-2H- pyrazolo[3,4-b]pyrazin-6-yl) amino)ethyl)-4-methylphenyl)- 2-(5-methylpyridin-3-yl) acetamide |
| 667 | 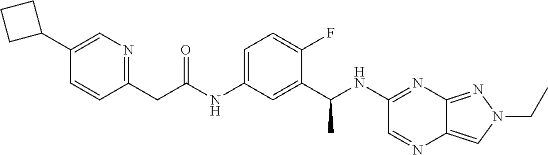 [SMILES: CCn1cc2ncc(N[C@@H](C)c3cc(NC(=O)Cc4ccc(C5CCC5)cn4)ccc3F)nc2n1] | (S)-2-(5-cyclobutylpyridin-2-yl)- N-(3-(1-((2-ethyl-2H- pyrazolo[3,4-b]pyrazin-6-yl) amino)ethyl)-4-fluorophenyl) acetamide |
| 668 | 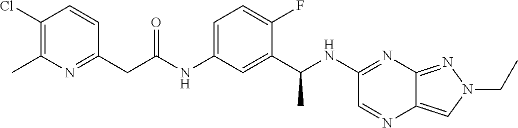 [SMILES: CCn1cc2ncc(N[C@@H](C)c3cc(NC(=O)Cc4ccc(Cl)c(C)n4)ccc3F)nc2n1] | (S)-2-(5-chloro-6- methylpyridin-2-yl)-N-(3-(1-((2- ethyl-2H-pyrazolo[3,4-b] pyrazin-6-yl)amino)ethyl)-4- fluorophenyl)acetamide |
| 669 | 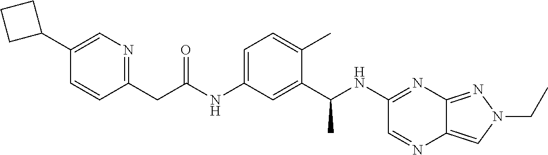 [SMILES: CCn1cc2ncc(N[C@@H](C)c3cc(NC(=O)Cc4ccc(C5CCC5)cn4)ccc3C)nc2n1] | (S)-2-(5-cyclobutylpyridin-2-yl)- N-(3-(1-((2-ethyl 2H- pyrazolo[3,4-b]pyrazin-6-yl) amino)ethyl)-4-methylphenyl) acetamide |
| 670 | 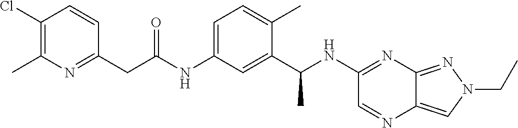 [SMILES: CCn1cc2ncc(N[C@@H](C)c3cc(NC(=O)Cc4ccc(Cl)c(C)n4)ccc3C)nc2n1] | (S)-2-(5-chloro-6- methylpyridin-2-yl)-N-(3-(1-((2- ethyl-2H-pyrazolo[3,4-b] pyrazin-6-yl)amino)ethyl)-4- methylphenyl)acetamide |
| 671 | 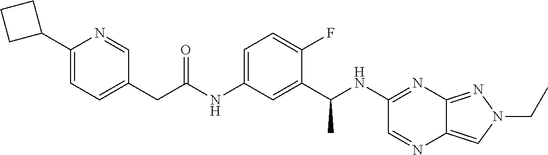 [SMILES: CCn1cc2ncc(N[C@@H](C)c3cc(NC(=O)Cc4ccc(C5CCC5)nc4)ccc3F)nc2n1] | (S)-2-(6-cyclobutylpyridin-3-yl)- N-(3-(1-((2-ethyl-2H- pyrazolo[3,4-b]pyrazin-6-yl) amino)ethyl)-4-fluorophenyl) acetamide |
| 672 | 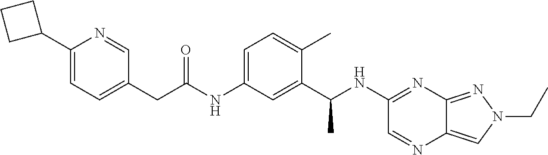 [SMILES: CCn1cc2ncc(N[C@@H](C)c3cc(NC(=O)Cc4ccc(C5CCC5)nc4)ccc3C)nc2n1] | (S)-2-(6-cyclobutylpyridin-3-yl)- N-(3-(1-((2-ethyl-2H- pyrazolo[3,4-b]pyrazin-6-yl) amino)ethyl)-4-methylphenyl) acetamide |
| 673 | 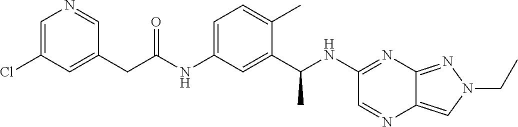 [SMILES: CCn1cc2ncc(N[C@@H](C)c3cc(NC(=O)Cc4cncc(Cl)c4)ccc3C)nc2n1] | (S)-2-(5-chloropyridin-3-yl)-N- (3-(1-((2-ethyl-2H-pyrazolo[3,4- b]pyrazin-6-yl)amino)ethyl)-4- methylphenyl)acetamide |
| 674 | 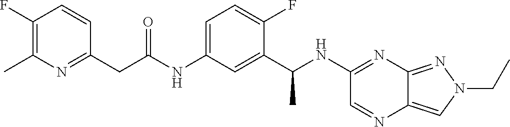 [SMILES: CCn1cc2ncc(N[C@@H](C)c3cc(NC(=O)Cc4ccc(F)c(C)n4)ccc3F)nc2n1] | (S)-N-(3-(1-((2-ethyl-2H- pyrazolo[3,4-b]pyrazin-6-yl) amino)ethyl)-4-fluorophenyl)-2- (5-fluoro-6-methylpyridin-2-yl) acetamide |
| 675 | 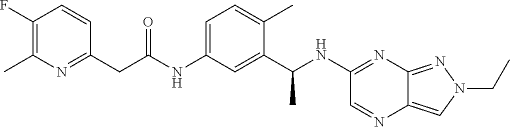 [SMILES: CCn1cc2ncc(N[C@@H](C)c3cc(NC(=O)Cc4ccc(F)c(C)n4)ccc3C)nc2n1] | (S)-N-(3-(1-((2-ethyl-2H- pyrazolo[3,4-b]pyrazin-6-yl) amino)ethyl)-4-methylphenyl)- 2-(5-fluoro-6-methylpyridin-2- yl)acetamide |
| 676 | 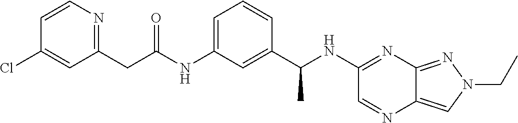 [SMILES: CCn1cc2ncc(N[C@@H](C)c3cccc(NC(=O)Cc4cc(Cl)ccn4)c3)nc2n1] | (S)-2-(4-chloropyridin-2-yl)-N- (3-(1-((2-ethyl-2H-pyrazolo[3,4- b]pyrazin-6-yl)amino)ethyl) phenyl)acetamide |
| 677 | 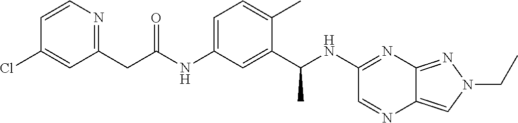 [SMILES: CCn1cc2ncc(N[C@@H](C)c3cc(NC(=O)Cc4cc(Cl)ccn4)ccc3C)nc2n1] | (6)-2-(4-chloropyridin-2-yl)-N- (3-(1-((2-ethyl-2H-pyrazolo[3,4- b]pyrazin-6-yl)amino)ethyl)-4- methylphenyl)acetamide |
| 678 | 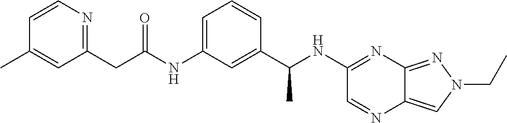 [SMILES: CCn1cc2ncc(N[C@@H](C)c3cccc(NC(=O)Cc4cc(C)ccn4)c3)nc2n1] | (S)-N-(3-(1-((2-ethyl-2H- pyrazolo[3,4-b]pyrazin-6-yl) amino)ethyl)phenyl)-2-(4- methylpyridin-2-yl)acetamide |
| 679 | 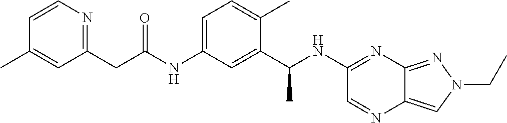 [SMILES: CCn1cc2ncc(N[C@@H](C)c3cc(NC(=O)Cc4cc(C)ccn4)ccc3C)nc2n1] | (S)-N-(3-(1-((2-ethyl-2H- pyrazolo[3,4-b]pyrazin-6-yl) amino)ethyl)-4-methylphenyl)- 2-(4-methylpyridin-2-yl) acetamide |
| 680 | 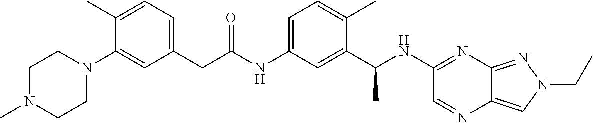 [SMILES: CCn1cc2ncc(N[C@@H](C)c3cc(NC(=O)Cc4ccc(C)c(N5CCN(C)CC5)c4)ccc3C)nc2n1] | (S)-N-(3-(1-((2-ethyl-2H- pyrazolo[3,4-b]pyrazin-6-yl) amino)ethyl)-4-methylphenyl)- 2-(4-methyl-3-(4- methylpiperazin-1-yl)phenyl) acetamide |
| 681 | 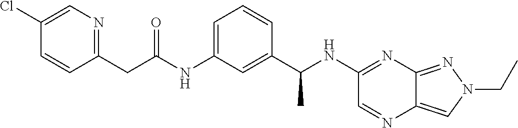 [SMILES: CCn1cc2ncc(N[C@@H](C)c3cccc(NC(=O)Cc4ccc(Cl)cn4)c3)nc2n1] | (S)-2-(5-chloropyridin-2-yl)-N- (3-(1-((2-ethyl-2H-pyrazolo[3,4- b]pyrazin-6-yl)amino)ethyl) phenyl)acetamide |
| 682 | 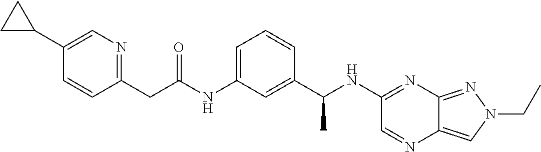 [SMILES: CCn1cc2ncc(N[C@@H](C)c3cccc(NC(=O)Cc4ccc(C5CC5)cn4)c3)nc2n1] | (S)-2-(5-cyclopropylpyridin-2- yl)-N-(3-(1-((2-ethyl-2H- pyrazolo[3,4-b]pyrazin-6-yl) amino)ethyl)phenyl)acetamide |
| 683 | 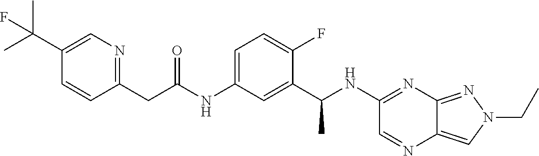 [SMILES: CCn1cc2ncc(N[C@@H](C)c3cc(NC(=O)Cc4ccc(C(C)(C)F)cn4)ccc3F)nc2n1] | (S)-N-(3-(1-((2-ethyl-2H- pyrazolo[3,4-b]pyrazin-6-yl) amino)ethyl)-4-fluorophenyl)-2- (5-(2-fluoropropan-2-yl)pyridin- 2-yl)acetamide |
| 684 | 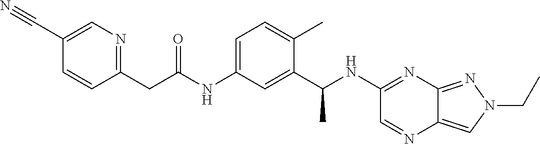 [SMILES: CCn1cc2ncc(N[C@@H](C)c3cc(NC(=O)Cc4ccc(C#N)cn4)ccc3C)nc2n1] | (S)-2-(5-cyanopyridin-2-yl)-N- (3-(1-((2-ethyl-2H-pyrazolo[3,4- b]pyrazin-6-yl)amino)ethyl)-4- methylphenyl)acetamide |
| 685 | 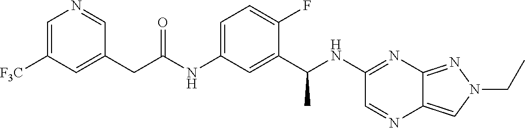 [SMILES: CCn1cc2ncc(N[C@@H](C)c3cc(NC(=O)Cc4cncc(C(F)(F)F)c4)ccc3F)nc2n1] | (S)-N-(3-(1-((2-ethyl-2H- pyrazolo[3,4-b]pyrazin-6-yl) amino)ethyl)-4-fluorophenyl)-2- (5-(trifluoromethyl)pyridin-3-yl) acetamide |
| 686 | 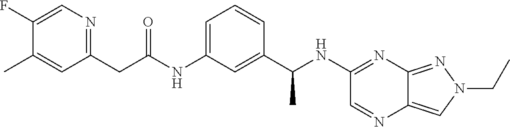 [SMILES: CCn1cc2ncc(N[C@@H](C)c3cccc(NC(=O)Cc4cc(C)c(F)cn4)c3)nc2n1] | (S)-N-(3-(1-((2-ethyl-2H- pyrazolo[3,4-b]pyrazin-6-yl) amino)ethyl)phenyl)-2-(5- fluoro-4-methylpyridin-2-yl) acetamide |
| 687 | 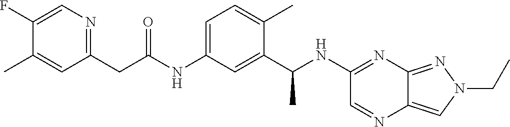 [SMILES: CCn1cc2ncc(N[C@@H](C)c3cc(NC(=O)Cc4cc(C)c(F)cn4)ccc3C)nc2n1] | (S)-N-(3-(1-((2-ethyl-2H- pyrazolo[3,4-b]pyrazin-6-yl) amino)ethyl)-4-methylphenyl)- 2-(5-fluoro-4-methylpyridin-2- yl)acetamide |
| 688 | 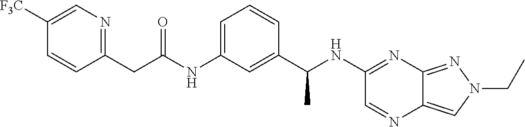 [SMILES: CCn1cc2ncc(N[C@@H](C)c3cccc(NC(=O)Cc4ccc(C(F)(F)F)cn4)c3)nc2n1] | (S)-N-(3-(1-((2-ethyl-2H- pyrazolo[3,4-b]pyrazin-6-yl) amino)ethyl)phenyl)-2-(5- (trifluoromethyl)pyridin-2-yl) acetamide |
| 689 | 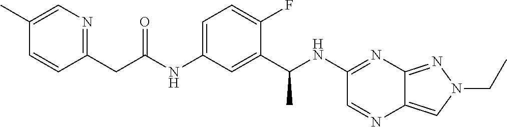 [SMILES: CCn1cc2ncc(N[C@@H](C)c3cc(NC(=O)Cc4ccc(C)cn4)ccc3F)nc2n1] | (S)-N-(3-(1-((2-ethyl-2H- pyrazolo[3,4-b]pyrazin-6-yl) amino)ethyl)-4-fluorophenyl)-2- (5-methylpyridin-2-yl) acetamide |
| 690 | 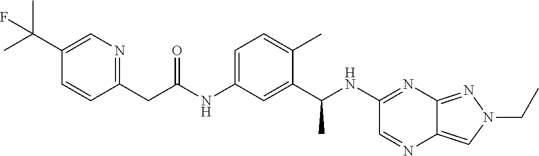 [SMILES: CCn1cc2ncc(N[C@@H](C)c3cc(NC(=O)Cc4ccc(C(C)(C)F)cn4)ccc3C)nc2n1] | (S)-N-(3-(1-((2-ethyl-2H- pyrazolo[3,4-b]pyrazin-6-yl) amino)ethyl)-4-methylphenyl)- 2-(5-(2-fluoropropan-2-yl) pyridin-2-yl)acetamide |
| 691 | 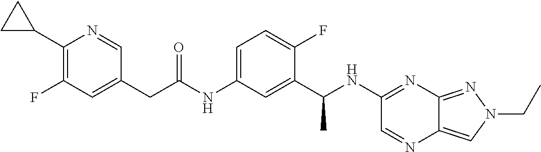 [SMILES: CCn1cc2ncc(N[C@@H](C)c3cc(NC(=O)Cc4cnc(C5CC5)c(F)c4)ccc3F)nc2n1] | (S)-2-(6-cyclopropyl-5- fluoropyridin-3-yl)-N-(3-(1-((2- ethyl-2H-pyrazolo[3,4-b] pyrazin-6-yl)amino)ethyl)-4- fluorophenyl)acetamide |
| 692 | 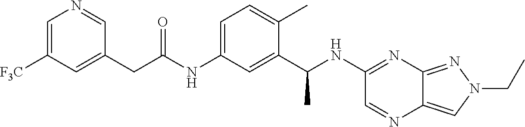 [SMILES: CCn1cc2ncc(N[C@@H](C)c3cc(NC(=O)Cc4cncc(C(F)(F)F)c4)ccc3C)nc2n1] | (S)-N-(3-(1-((2-ethyl-2H- pyrazolo[3,4-b]pyrazin-6-yl) amino)ethyl)-4-methylphenyl)- 2-(5-(trifluoromethyl)pyridin-3- yl)acetamide |
| 693 | 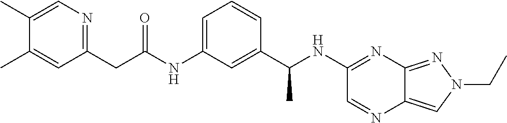 [SMILES: CCn1cc2ncc(N[C@@H](C)c3cccc(NC(=O)Cc4cc(C)c(C)cn4)c3)nc2n1] | (S)-2-(4,5-dimethylpyridin-2-yl)- N-(3-(1-((2-ethyl-2H- pyrazolo[3,4-b]pyrazin-6-yl) amino)ethyl)phenyl)acetamide |
| 694 | 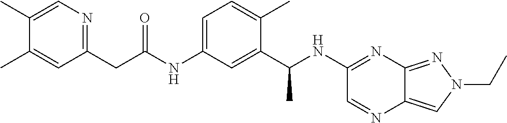 [SMILES: CCn1cc2ncc(N[C@@H](C)c3cc(NC(=O)Cc4cc(C)c(C)cn4)ccc3C)nc2n1] | (S)-2-(4,5-dimethylpyridin-2-yl)- N-(3-(1-((2-ethyl-2H- pyrazolo[3,4-b]pyrazin-6-yl) amino)ethyl)-4-methylphenyl) acetamide |
| 695 | 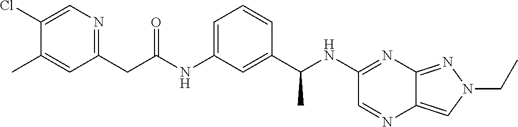 [SMILES: CCn1cc2ncc(N[C@@H](C)c3cccc(NC(=O)Cc4cc(C)c(Cl)cn4)c3)nc2n1] | (S)-2-(5-chloro-4- methylpyridin-2-yl)-N-(3-(1-((2- ethyl-2H-pyrazolo[3,4-b] pyrazin-6-yl)amino)ethyl) phenyl)acetamide |
| 696 | 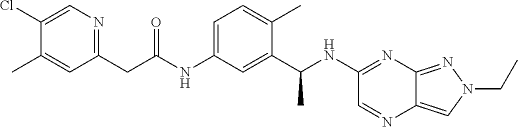 [SMILES: CCn1cc2ncc(N[C@@H](C)c3cc(NC(=O)Cc4cc(C)c(Cl)cn4)ccc3C)nc2n1] | (S)-2-(5-chloro-4- methylpyridin-2-yl)-N-(3-(1-((2- ethyl-2H-pyrazolo[3,4-b] pyrazin-6-yl)amino)ethyl)-4- methylphenyl)acetamide |
| 697 | 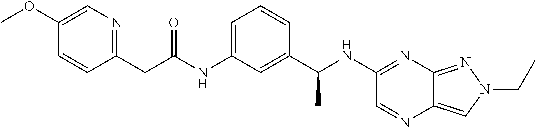 [SMILES: CCn1cc2ncc(N[C@@H](C)c3cccc(NC(=O)Cc4ccc(OC)cn4)c3)nc2n1] | (S)-N-(3-(1-((2-ethyl-2H- pyrazolo[3,4-b]pyrazin-6-yl) amino)ethyl)phenyl)-2-(5- methoxypyridin-2-yl)acetamide |
| 698 | 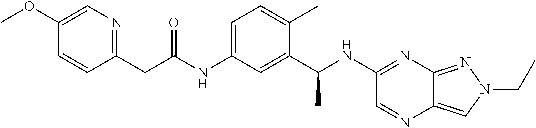 [SMILES: CCn1cc2ncc(N[C@@H](C)c3cc(NC(=O)Cc4ccc(OC)cn4)ccc3C)nc2n1] | (S)-N-(3-(1-((2-ethyl-2H- pyrazolo[3,4-b]pyrazin-6-yl) amino)ethyl)-4-methylphenyl)- 2-(5-methoxypyridin-2-yl) acetamide |
| 699 | 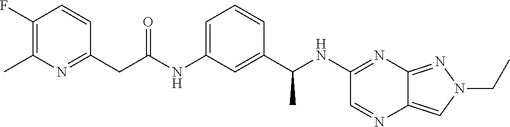 [SMILES: CCn1cc2ncc(N[C@@H](C)c3cccc(NC(=O)Cc4ccc(F)c(C)n4)c3)nc2n1] | (S)-N-(3-(1-((2-ethyl-2H- pyrazolo[3,4-b]pyrazin-6-yl) ainino)ethyl)phenvl)-2-(5- fluoro-6-methylpyridin-2-yl) acetamide |
| 700 | 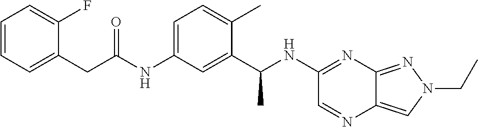 [SMILES: CCn1cc2ncc(N[C@@H](C)c3cc(NC(=O)Cc4ccccc4F)ccc3C)nc2n1] | (S)-N-(3-(1-((2-ethyl-2H- pyrazolo[3,4-b]pyrazin-6-yl) amino)ethyl)-4-methylphenyl)- 2-(2-fluorophenyl)acetamide |
| 701 | 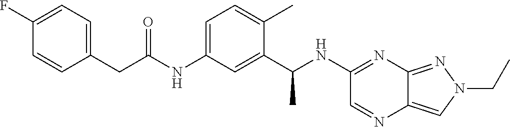 [SMILES: CCn1cc2ncc(N[C@@H](C)c3cc(NC(=O)Cc4ccc(F)cc4)ccc3C)nc2n1] | (S)-N-(3-(1-((2-ethyl-2H- pyrazolo[3,4-b]pyrazin-6-yl) amino)ethyl)-4-methylphenyl)- 2-(4-fluorophenyl)acetamide |
| 702 | 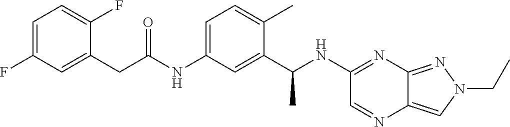 [SMILES: CCn1cc2ncc(N[C@@H](C)c3cc(NC(=O)Cc4cc(F)ccc4F)ccc3C)nc2n1] | (S)-2-(2,5-difluorophenyl)-N-(3- (1-((2-ethyl-2H-pyrazolo[3,4-b] pyrazin-6-yl)amino)ethyl)-4- methylphenyl)acetamide |
| 703 | 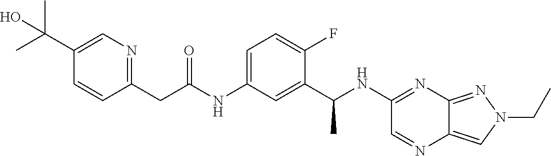 [SMILES: CCn1cc2ncc(N[C@@H](C)c3cc(NC(=O)Cc4ccc(C(C)(C)O)cn4)ccc3F)nc2n1] | (S)-N-(3-(1-((2-ethyl-2H- pyrazolo[3,4-b]pyrazin-6-yl) amino)ethyl)-4-fluorophenyl)-2- (5-(2-hydroxypropan-2-yl) pyridin-2-yl)acetamide |
| 704 | 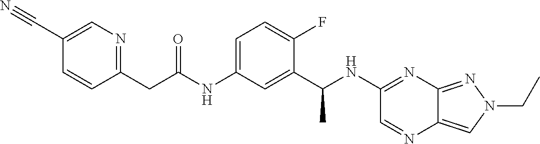 [SMILES: CCn1cc2ncc(N[C@@H](C)c3cc(NC(=O)Cc4ccc(C#N)cn4)ccc3F)nc2n1] | (S)-2-(5-cyanopyridin-2-yl)-N- (3-(1-((2-ethyl-2H-pyrazolo[3,4- b]pyrazin-6-yl)amino)ethyl)-4- fluorophenyl)acetamide |
| 705 | 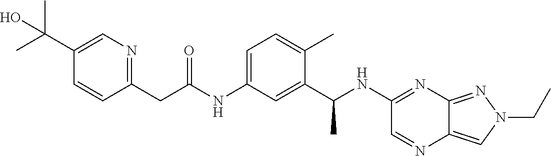 [SMILES: CCn1cc2ncc(N[C@@H](C)c3cc(NC(=O)Cc4ccc(C(C)(C)O)cn4)ccc3C)nc2n1] | (S)-N-(3-(1-((2-ethyl-2H- pyrazolo[3,4-b]pyrazin-6-yl) amino)ethyl)-4-methylphenyl)- 2-(5-(2-hydroxypropan-2-yl) pyridin-2-yl)acetamide |
| 706 | 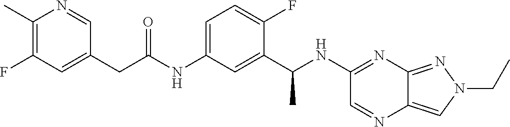 [SMILES: CCn1cc2ncc(N[C@@H](C)c3cc(NC(=O)Cc4cnc(C)c(F)c4)ccc3F)nc2n1] | (S)-N-(3-(1-((2-ethyl-2H- pyrazolo[3,4-b]pyrazin-6-yl) amino)ethyl)-4-fluorophenyl)-2- (5-fluoro-6-methylpyridin-3-yl) acetamide |
| 707 | 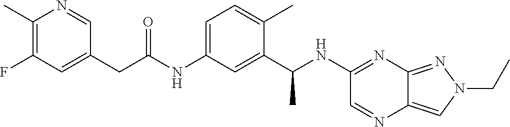 [SMILES: CCn1cc2ncc(N[C@@H](C)c3cc(NC(=O)Cc4cnc(C)c(F)c4)ccc3C)nc2n1] | (S)-N-(3-(1-((2-ethyl-2H- pyrazolo[3,4-b]pyrazin-6-yl) amino)ethyl)-4-methylphenyl)- 2-(5-fluoro-6-methylpyridin-3- yl)acetamide |
| 708 | 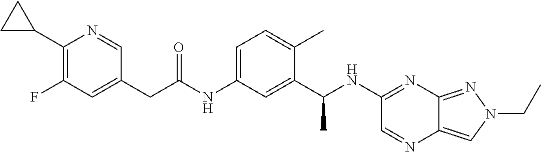 [SMILES: CCn1cc2ncc(N[C@@H](C)c3cc(NC(=O)Cc4cnc(C5CC5)c(F)c4)ccc3C)nc2n1] | (S)-2-(6-cyclopropyl-5- fluoropyridin-3-yl)-N-(3-(1-((2- ethyl-2H-pyrazolo[3,4-b] pyrazin-6-yl)amino)ethyl)-4- methylphenyl)acetamide |
| 709 | 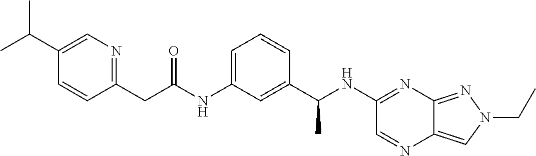 [SMILES: CCn1cc2ncc(N[C@@H](C)c3cccc(NC(=O)Cc4ccc(C(C)C)cn4)c3)nc2n1] | (S)-N-(3-(1-((2-ethyl-2H- pyrazolo[3,4-b]pyrazin-6-yl) amino)ethyl)phenyl)-2-(5- isopropylpyridin-2-yl)acetamide |
| 710 | 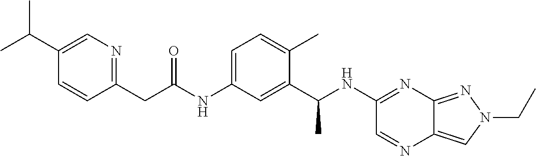 [SMILES: CCn1cc2ncc(N[C@@H](C)c3cc(NC(=O)Cc4ccc(C(C)C)cn4)ccc3C)nc2n1] | (S)-N-(3-(1-((2-ethyl-2H- pyrazolo[3,4-b]pyrazin-6-yl) amino)ethyl)-4-methylphenyl)- 2-(5-isopropylpyridin-2-yl) acetamide |
| 711 | 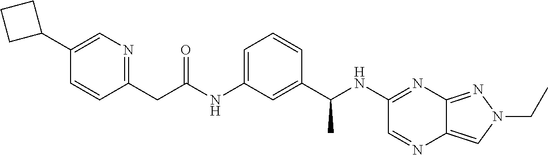 [SMILES: CCn1cc2ncc(N[C@@H](C)c3cccc(NC(=O)Cc4ccc(C5CCC5)cn4)c3)nc2n1] | (S)-2-(5-cyclobutylpyridin-2-yl)- N-(3-(1-((2-ethyl-2H- pyrazolo[3,4-b]pyrazin-6-yl) amino)ethyl)phenyl)acetamide |
| 712 | 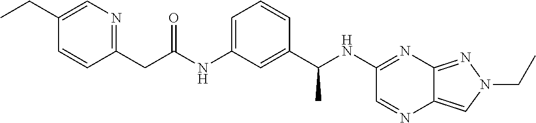 [SMILES: CCc1ccc(CC(=O)Nc2cccc([C@H](C)Nc3cnc4cn(CC)nc4n3)c2)nc1] | (S)-N-(3-(1-((2-ethyl-2H- pyrazolo[3,4-b]pyrazin-6-yl) amino)ethyl)phenyl)-2-(5- ethylpyridin-2-yl)acetamide |
| 713 | 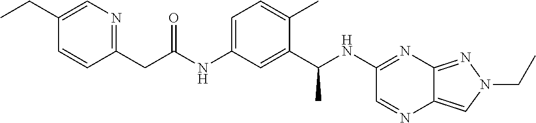 [SMILES: CCc1ccc(CC(=O)Nc2ccc(C)c([C@H](C)Nc3cnc4cn(CC)nc4n3)c2)nc1] | (S)-N-(3-(1-((2-ethyl-2H- pyrazolo[3,4-b]pyrazin-6-yl) amino)ethyl)-4-methylphenyl)- 2-(5-ethylpyridin-2-yl) acetamide |
| 714 | 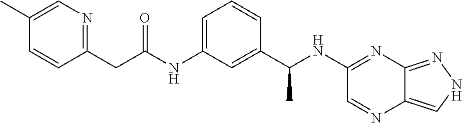 [SMILES: Cc1ccc(CC(=O)Nc2cccc([C@H](C)Nc3cnc4c[nH]nc4n3)c2)nc1] | (S)-N-(3-(1-((2H-pyrazolo[3,4- b]pyrazin-6-yl)amino)ethyl) phenyl)-2-(5-methylpyridin-2- yl)acetamide |
| 715 | 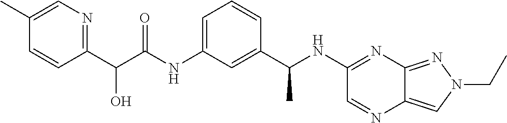 [SMILES: CCn1cc2ncc(N[C@@H](C)c3cccc(NC(=O)C(O)c4ccc(C)cn4)c3)nc2n1] | N-(3-((S)-1-((2-ethyl-2H- pyrazolo[3,4-b]pyrazin-6-yl) amino)ethyl)phenyl)-2-hydroxy- 2-(5-methylpyridin-2-yl) acetamide |
| 716 | 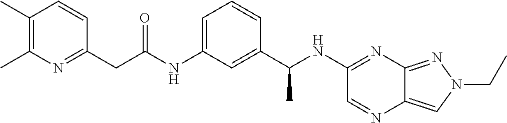 [SMILES: CCn1cc2ncc(N[C@@H](C)c3cccc(NC(=O)Cc4ccc(C)c(C)n4)c3)nc2n1] | (S)-2-(5,6-dimethylpyridin-2-yl)- N-(3-(1-((2-ethyl-2H- pyrazolo[3,4-b]pyrazin-6-yl) amino)ethyl)phenyl)acetamide |
| 717 | 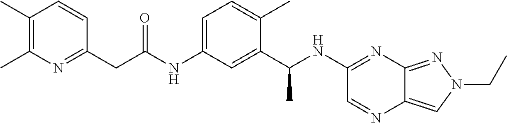 [SMILES: CCn1cc2ncc(N[C@@H](C)c3cc(NC(=O)Cc4ccc(C)c(C)n4)ccc3C)nc2n1] | (S)-2-(5,6-dimethylpyridin-2-yl)- N-(3-(1-((2-ethyl-2H- pyrazolo[3,4-b]pyrazin-6-yl) amino)ethyl)-4-methylphenyl) acetamide |
| 718 | 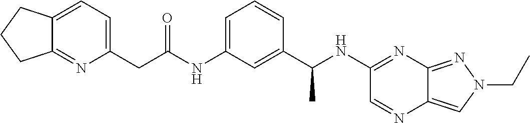 [SMILES: CCn1cc2ncc(N[C@@H](C)c3cccc(NC(=O)Cc4ccc5c(n4)CCC5)c3)nc2n1] | (S)-2-(6,7-dihydro-5H- cyclopenta[b]pyridin-2-yl)-N- (3-(1-((2-ethyl-2H-pyrazolo[3,4- b]pyrazin-6-yl)amino)ethyl) phenyl)acetamide |
| 719 | 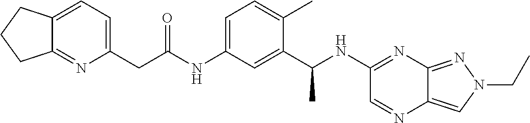 [SMILES: CCn1cc2ncc(N[C@@H](C)c3cc(NC(=O)Cc4ccc5c(n4)CCC5)ccc3C)nc2n1] | (S)-2-(6,7-dihydro-5H- cyclopenta[b]pyridin-2-yl)-N- (3-(1-((2-ethyl-2H-pyrazolo[3,4- b]pyrazin-6-yl)amino)ethyl)-4- methylphenyl)acetamide |
| 720 | 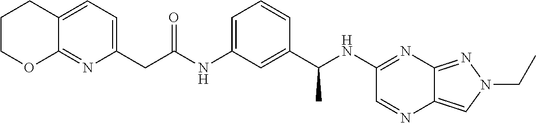 [SMILES: CCn1cc2ncc(N[C@@H](C)c3cccc(NC(=O)Cc4ccc5c(n4)OCCC5)c3)nc2n1] | (S)-2-(3,4-dihydro-2H- pyrano[2,3-b]pyridin-7-yl)-N- (3-(1-((2-ethyl-2H-pyrazolo[3,4- b]pyrazin-6-yl)amino)ethyl) phenyl)acetamide |
| 721 | 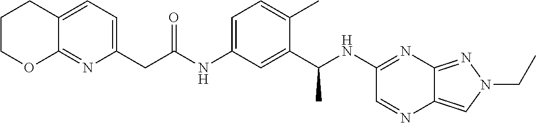 [SMILES: CCn1cc2ncc(N[C@@H](C)c3cc(NC(=O)Cc4ccc5c(n4)OCCC5)ccc3C)nc2n1] | (S)-2-(3,4-dihydro-2H- pyrano[2,3-b]pyridin-7-yl)N- (3-(1-((2-ethyl-2H-pyrazolo[3,4- b]pyrazin-6-yl)amino)ethyl)-4- methylphenyl)acetamide |
| 722 | 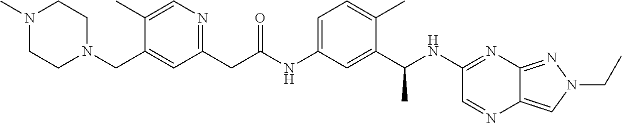 [SMILES: CCn1cc2ncc(N[C@@H](C)c3cc(NC(=O)Cc4cc(CN5CCN(C)CC5)c(C)cn4)ccc3C)nc2n1] | (S)-N-(3-(1-((2-ethyl-2H- pyrazolo[3,4-b]pyrazin-6-yl) amino)ethyl)-4-methylphenyl)- 2-(5-methyl-4-((4- methylpiperazin-1-yl)methyl) pyridin-2-yl)acetamide |
| 723 | 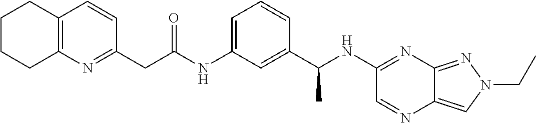 [SMILES: CCn1cc2ncc(N[C@@H](C)c3cccc(NC(=O)Cc4ccc5c(n4)CCCC5)c3)nc2n1] | (S)-N-(3-(1-((2-ethyl-2H- pyrazolo[3,4-b]pyrazin-6-yl) amino)ethyl)phenyl)-2-(5,6,7,8- tetrahydroquinolin-2-yl) acetamide |
| 724 | 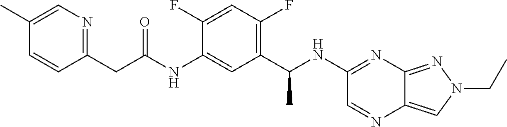 [SMILES: CCn1cc2ncc(N[C@@H](C)c3cc(NC(=O)Cc4ccc(C)cn4)c(F)cc3F)nc2n1] | (S)-N-(5-(1-((2-ethyl-2H- pyrazolo[3,4-b]pyrazin-6-yl) amino)ethyl)-2,4- difluorophenyl)-2-(5- methylpyridin-2-yl)acetamide |
| 725 | 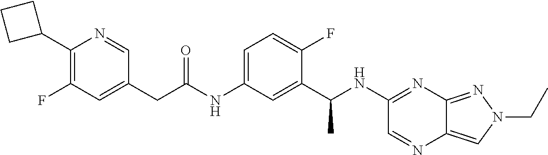 [SMILES: CCn1cc2ncc(N[C@@H](C)c3cc(NC(=O)Cc4cnc(C5CCC5)c(F)c4)ccc3F)nc2n1] | (S)-2-(6-cyclobutyl-5- fluoropyridin-3-yl)-N-(3-(1-((2- ethyl-2H-pyrazolo[3,4-b] pyrazin-6-yl)amino)ethyl)-4- fluorophenyl)acetamide |
| 726 | 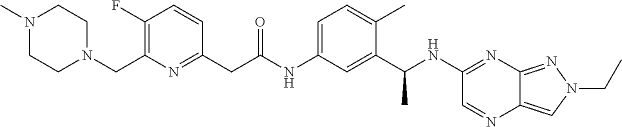 [SMILES: CCn1cc2ncc(N[C@@H](C)c3cc(NC(=O)Cc4ccc(F)c(CN5CCN(C)CC5)n4)ccc3C)nc2n1] | (S)-N-(3-(1-((2-ethyl-2H- pyrazolo[3,4-b]pyrazin-6-yl) amino)ethyl)-4-methylphenyl)- 2-(5-fluoro-6-((4- methylpiperazin-1-yl)methyl) pyridin-2-yl)acetamide |
| 727 | 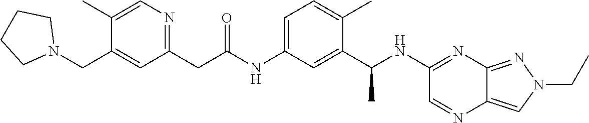 [SMILES: CCn1cc2ncc(N[C@@H](C)c3cc(NC(=O)Cc4cc(CN5CCCC5)c(C)cn4)ccc3C)nc2n1] | (S)-N-(3-(1-((2-ethyl-2H- pyrazolo[3,4-b]pyrazin-6-yl) amino)ethyl)-4-methylphenyl)- 2-(5-methyl-4-(pyrrolidin-1- ylmethyl)pyridin-2-yl)acetamide |
| 728 | 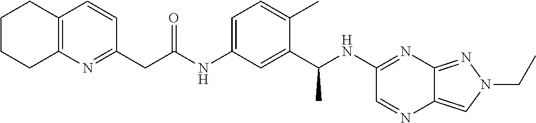 [SMILES: CCn1cc2ncc(N[C@@H](C)c3cc(NC(=O)Cc4ccc5c(n4)CCCC5)ccc3C)nc2n1] | (S)-N-(3-(1-((2-ethyl-2H- pyrazolo[3,4-b]pyrazin-6-yl) amino)ethyl)-4-methylphenyl)- 2-(5,6,7,8-tetrahydroquinolin-2- yl)acetamide |
| 729 | 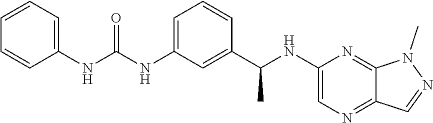 [SMILES: C[C@H](Nc1cnc2cnn(C)c2n1)c1cccc(NC(=O)Nc2ccccc2)c1] | (S)-1-(3-(1-((1-methyl-1H- pyrazolo[3,4]pyrazin-6-yl) amino)ethyl)phenyl)-3- phenylurea |
| 730 | 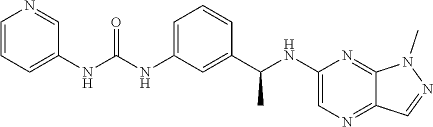 [SMILES: C[C@H](Nc1cnc2cnn(C)c2n1)c1cccc(NC(=O)Nc2cccnc2)c1] | (S)-1-(3-(1-((1-methyl-1H- pyrazolo[3,4-b]pyrazin-6-yl) amino)ethyl)phenyl)-3-(pyridin- 3-yl)urea |
| 731 | 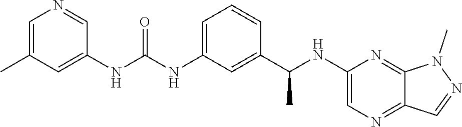 [SMILES: Cc1cncc(NC(=O)Nc2cccc([C@H](C)Nc3cnc4cnn(C)c4n3)c2)c1] | (S)-1-(3-(1-((1-methyl-1H- pyrazolo[3,4-b]pyrazin-6-yl) amino)ethyl)phenyl)-3-(5- methylpyridin-3-yl)urea |
| 732 | 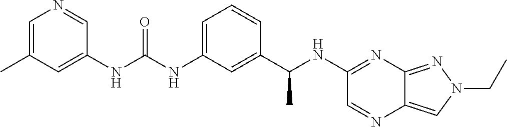 [SMILES: CCn1cc2ncc(N[C@@H](C)c3cccc(NC(=O)Nc4cncc(C)c4)c3)nc2n1] | (S)-1-(3-(1-((2-ethyl-2H- pyrazolo[3,4-b]pyrazin-6-yl) amino)ethyl)phenyl)-3-(5- methylpyridin-3-yl)urea |
| 733 | 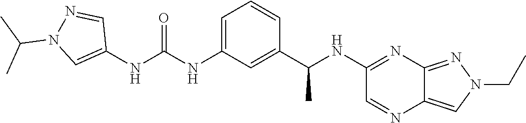 [SMILES: CCn1cc2ncc(N[C@@H](C)c3cccc(NC(=O)Nc4cnn(C(C)C)c4)c3)nc2n1] | (S)-1-(3-(1-((2-ethyl-2H- pyrazolo[3,4-b]pyrazin-6-yl) amino)ethyl)phenyl)-3-(1- isopropyl-1H-pyrazol-4-yl)urea |
| 734 | 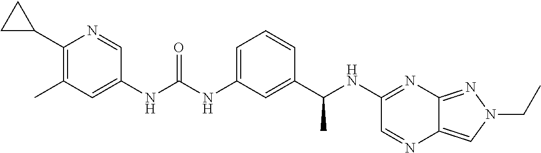 [SMILES: CCn1cc2ncc(N[C@@H](C)c3cccc(NC(=O)Nc4cnc(C5CC5)c(C)c4)c3)nc2n1] | (S)-1-(6-cyclopropyl-5- methylpyridin-3-yl)-3-(3-(1-((2- ethyl-2H-pyrazolo[3,4-b] pyrazin-6-yl)amino)ethyl) phenyl)urea |
| 735 | 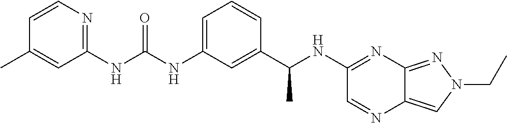 [SMILES: CCn1cc2ncc(N[C@@H](C)c3cccc(NC(=O)Nc4cc(C)ccn4)c3)nc2n1] | (S)-1-(3-(1-((2-ethyl-2H- pyrazolo[3,4-b]pyrazin-6-yl) amino)ethyl)phenyl)-3-(4- methylpyridin-2-yl)urea |
| 736 | 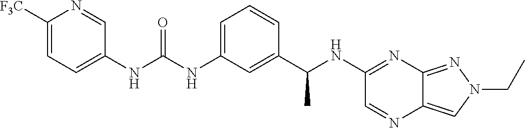 [SMILES: CCn1cc2ncc(N[C@@H](C)c3cccc(NC(=O)Nc4ccc(C(F)(F)F)nc4)c3)nc2n1] | (S)-1-(3-(1-((2-ethyl-2H- pyrazolo[3,4-b]pyrazin-6-yl) amino)ethyl)phenyl)-3-(6- (trifluoromethyl)pyridin-3-yl) urea |
| 737 | 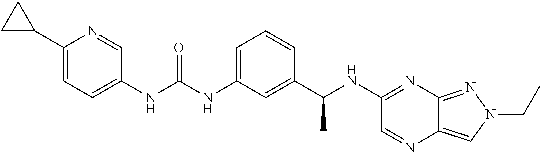 [SMILES: CCn1cc2ncc(N[C@@H](C)c3cccc(NC(=O)Nc4ccc(C5CC5)nc4)c3)nc2n1] | (S)-1-(6-cyclopropylpyridin-3- yl)-3-(3-(1-((2-ethyl-2H- pyrazolo[3,4-b]pyrazin-6-yl) amino)ethyl)phenyl)urea |
| 738 | 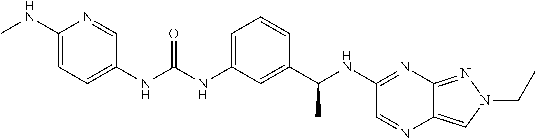 [SMILES: CCn1cc2ncc(N[C@@H](C)c3cccc(NC(=O)Nc4ccc(NC)nc4)c3)nc2n1] | (S)-1-(3-(1-((2-ethyl-2H- pyrazolo[3,4-b]pyrazin-6-yl) amino)ethyl)phenyl)-3-(6- (methylamino)pyridin-3-yl)urea |
| 739 | 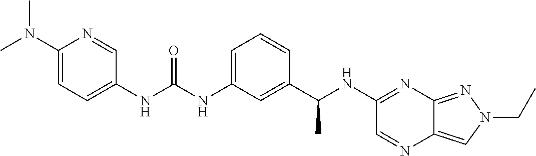 [SMILES: CCn1cc2ncc(N[C@@H](C)c3cccc(NC(=O)Nc4ccc(N(C)C)nc4)c3)nc2n1] | (S)-1-(6-(dimethylamino) pyridin-3-yl)-3-(3-(1-((2-ethyl- 2H-pyrazolo[3,4-b]pyrazin-6-yl) amino)ethyl)phenyl)urea |
| 740 | 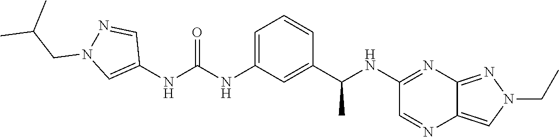 [SMILES: CCn1cc2ncc(N[C@@H](C)c3cccc(NC(=O)Nc4cnn(CC(C)C)c4)c3)nc2n1] | (S)-1-(3-(1-((2-ethyl-2H- pyrazolo[3,4-b]pyrazin-6-yl) amino)ethyl)phenyl)-3-(1- isobutyl-1H-pyrazol-4-yl)urea |
| 741 | 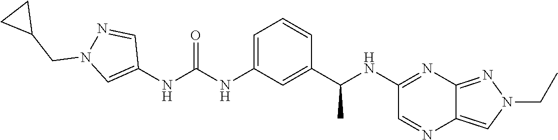 [SMILES: CCn1cc2ncc(N[C@@H](C)c3cccc(NC(=O)Nc4cnn(CC5CC5)c4)c3)nc2n1] | (S)-1-(1-(cyclopropylmethyl)- 1H-pyrazol-4-yl)-3-(3-(1-((2- ethyl-2H-pyrazolo[3,4-b] pyrazin-6-yl)amino)ethyl) phenyl)urea |
| 742 | 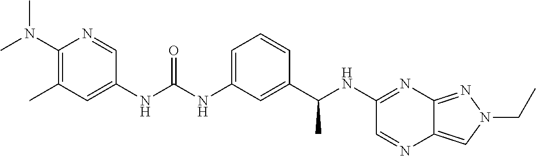 [SMILES: CCn1cc2ncc(N[C@@H](C)c3cccc(NC(=O)Nc4cnc(N(C)C)c(C)c4)c3)nc2n1] | (S)-1-(6-(dimethylamino)-5- methylpyridin-3-yl)-3-(3-(1-((2- ethyl-2H-pyrazolo[3,4-b] pyrazin-6-yl)amino)ethyl) phenyl)urea |
| 743 | 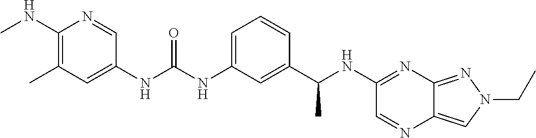 [SMILES: CCn1cc2ncc(N[C@@H](C)c3cccc(NC(=O)Nc4cnc(NC)c(C)c4)c3)nc2n1] | (S)-1-(3-(1-((2-ethyl-2H- pyrazolo[3,4-b]pyrazin-6-yl) amino)ethyl)phenyl)-3-(5- methyl-6-(methylamino)pyridin- 3-yl)urea |
| 744 | 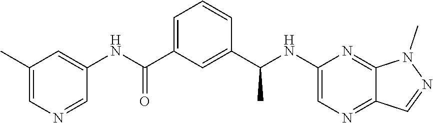 [SMILES: Cc1cncc(NC(=O)c2cccc([C@H](C)Nc3cnc4cnn(C)c4n3)c2)c1] | (S)-3-(1-((1-methyl-H- pyrazolo[3,4-b]pyrazin-6-yl) amino)ethyl)-N-(5- methylpyridin-3-yl)benzamide |
| 745 | 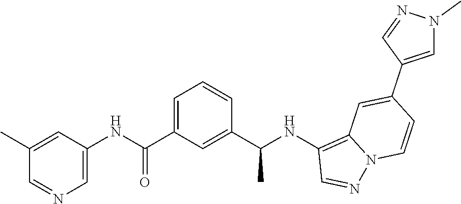 | (S)-5-methyl-N-(3-(1-((5-(1- methyl-1H-pyrazol-4-yl) pyrazolo[1,5-a]pyridin-3-yl) amino)ethyl)phenyl) nicotinamide |
| 746 | 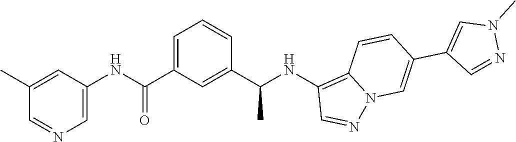 | (S)-5-methyl-N-(3-(1-((6-(1- methyl-1H-pyrazol-4-yl) pyrazolo[1,5-a]pyridin-3-yl) amino)ethyl)phenyl) nicotinamide |
| 747 | 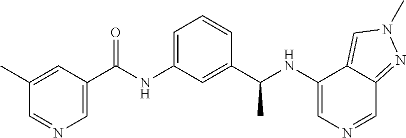 | (S)-5-methyl-N-(3-(1-((2- methyl-2H-pyrazolo[3,4-c] pyridin-4-yl)amino)ethyl) phenyl)nicotinamide |
| 748 | 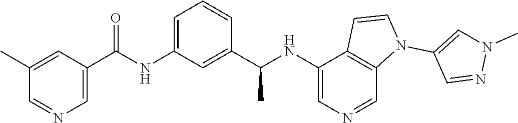 | (S)-5-methyl-N-(3-(1-((1-(1- methyl-1H-pyrazol-4-yl)-1H- pyrrolo[2,3-c]pyridin-4-yl) amino)ethyl)phenyl) nicotinamide |
| 749 | 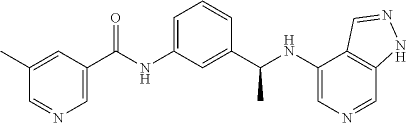 [SMILES: Cc1cncc(C(=O)Nc2cccc([C@H](C)Nc3cncc4[nH]ncc34)c2)c1] | (S)-N-(3-(1-((1H-pyrazolo[3,4- c]pyridin-4-yl)amino)ethyl) phenyl)-5-methylnicotinamide |
| 750 | 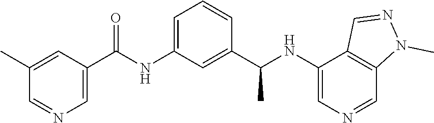 | (S)-5-methyl-N-(3-(1-((1- methyl-1H-pyrazolo[3,4-c] pyridin-4-yl)amino)ethyl) phenyl)nicotinamide |
| 751 | 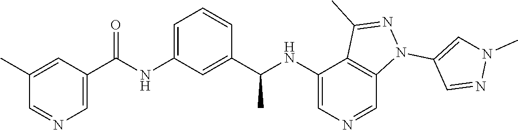 | (S)-5-methyl-N-(3-(1-((3- methyl-1-(1-methyl-1H-pyrazol- 4-yl)-1H-pyrazolo[3,4-c] pyridin-4-yl)amino)ethyl) phenyl)nicotinamide |

“Isomer” is used herein to encompass all chiral, diastereomeric or racemic forms of a structure, unless a particular stereochemistry or isomeric form is specifically indicated. Such compounds can be enriched or resolved optical isomers at any or all asymmetric atoms as are apparent from the depictions, at any degree of enrichment. Both racemic and diastereomeric mixtures, as well as the individual optical isomers can be synthesized so as to be substantially free of their enantiomeric or diastereomeric partners, and these are all within the scope of certain embodiments of the disclosure. The isomers resulting from the presence of a chiral center comprise a pair of non-superimposable isomers that are called “enantiomers.” Single enantiomers of a pure compound are optically active (i.e., they are capable of rotating the plane of plane polarized light and designated R or S).

“Isolated optical isomer” means a compound which has been substantially purified from the corresponding optical isomer(s) of the same formula. For example, the isolated isomer may be at least about 80%, at least 80% or at least 85% pure. In other embodiments, the isolated isomer is at least 90% pure or at least 98% pure, or at least 99% pure by weight.

“Substantially enantiomerically or diasteromerically” pure means a level of enantiomeric or diastereomeric enrichment of one enantiomer with respect to the other enantiomer or diasteromer of at least about 80%, and more specifically in excess of 80%, 85%, 90%, 95%, 98%, 99%, 99.5% or 99.9%.

The terms “racemate” and “racemic mixture” refer to an equal mixture of two enantiomers. A racemate is labeled “(±)” because it is not optically active (i.e., will not rotate plane-polarized light in either direction since its constituent enantiomers cancel each other out).

A “hydrate” is a compound that exists in combination with water molecules. The combination can include water in stoichiometric quantities, such as a monohydrate or a dihydrate, or can include water in random amounts. As the term is used herein a “hydrate” refers to a solid form; that is, a compound in a water solution, while it may be hydrated, is not a hydrate as the term is used herein.

A “solvate” is similar to a hydrate except that a solvent other that water is present. For example, methanol or ethanol can form an “alcoholate”, which can again be stoichiometric or non-stoichiometric. As the term is used herein a “solvate” refers to a solid form; that is, a compound in a solvent solution, while it may be solvated, is not a solvate as the term is used herein.

“Isotope” refers to atoms with the same number of protons but a different number of neutrons, and an isotope of a compound having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), includes any such compound wherein one or more atoms are replaced by an isotope of that atom. For example, carbon 12, the most common form of carbon, has six protons and six neutrons, whereas carbon 13 has six protons and seven neutrons, and carbon 14 has six protons and eight neutrons. Hydrogen has two stable isotopes, deuterium (one proton and one neutron) and tritium (one proton and two neutrons). While fluorine has a number of isotopes, fluorine 19 is longest-lived. Thus, an isotope of a compound having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII), (XIX), or (XX) includes, but is not limited to, compounds having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII), (XIX), or (XX) wherein one or more carbon 12 atoms are replaced by carbon-13 and/or carbon-14 atoms, wherein one or more hydrogen atoms are replaced with deuterium and/or tritium, and/or wherein one or more fluorine atoms are replaced by fluorine-19.

“Salt” generally refers to an organic compound, such as a carboxylic acid or an amine, in ionic form, in combination with a counter ion. For example, salts formed between acids in their anionic form and cations are referred to as “acid addition salts”. Conversely, salts formed between bases in the cationic form and anions are referred to as “base addition salts.”

The term “pharmaceutically acceptable” refers an agent that has been approved for human consumption and is generally non-toxic. For example, the term “pharmaceutically acceptable salt” refers to nontoxic inorganic or organic acid and/or base addition salts (see, e.g., Lit et al., Salt Selection for Basic Drugs, Int. J. Pharm., 33, 201-217, 1986) (incorporated by reference herein).

Pharmaceutically acceptable base addition salts of compounds of the disclosure include, for example, metallic salts including alkali metal, alkaline earth metal, and transition metal salts such as, for example, calcium, magnesium, potassium, sodium, and zinc salts. Pharmaceutically acceptable base addition salts also include organic salts made from basic amines such as, for example, N,N′dibenzylethylenediamine, chloroprocaine, choline, diethanolamine, ethylenediamine, meglumine (N-methylglucamine), and procaine.

Pharmaceutically acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, aromatic aliphatic, heterocyclic, carboxylic, and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenyl acetic, mandelic, hippuric, malonic, oxalic, embonic (pamoic), methanesulfonic, ethanesulfonic, benzenesulfonic, panthothenic, trifluoromethanesulfonic, 2-hydroxyethanesulfonic, p-toluenesulfonic, sulfanilic, cyclohexylaminosulfonic, stearic, alginic, βhydroxybutyric, salicylic, -galactaric, and galacturonic acid.

Although pharmaceutically unacceptable salts are not generally useful as medicaments, such salts may be useful, for example as intermediates in the synthesis of compounds having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), for example in their purification by recrystallization.

In certain embodiments, the disclosure provides a pharmaceutical composition comprising a compound having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), or a pharmaceutically acceptable isomer, racemate, hydrate, solvate, tautomer, isotope, or salt thereof, together with at least one pharmaceutically acceptable carrier, diluent, or excipient. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid, or lower alkyl ethers of cellulose, silicic acid, fatty acids, fatty acid amines, fatty acid monoglycerides and diglycerides, pentaerythritol fatty acid esters, polyoxyethylene, hydroxymethylcellulose, and polyvinylpyrrolidone. Similarly, the carrier or diluent can include any sustained release material known in the art, such as glyceryl monostearate or glyceryl distearate, alone or mixed with a wax.

As used herein, the term “pharmaceutical composition” refers to a composition containing one or more of the compounds described herein, or a pharmaceutically acceptable isomer, racemate, hydrate, solvate, homolog or salt thereof, formulated with a pharmaceutically acceptable carrier, which can also include other additives, and manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein. Conventional procedures and ingredients for the selection and preparation of suitable formulations are described, for example, in Remington: The Science and Practice of Pharmacy, 21st Ed., Gennaro, Ed., Lippencott Williams & Wilkins (2005) and in The United States Pharmacopeia: The National Formulary (USP 36 NF31), published in 2013.

In another embodiment, there are provided methods of making a composition of a compound described herein including formulating a compound of the disclosure with a pharmaceutically acceptable carrier or diluent. In some embodiments, the pharmaceutically acceptable carrier or diluent is suitable for oral administration. In some such embodiments, the methods can further include the step of formulating the composition into a tablet or capsule. In other embodiments, the pharmaceutically acceptable carrier or diluent is suitable for parenteral administration. In some such embodiments, the methods further include the step of lyophilizing the composition to form a lyophilized preparation.

As used herein, the term “pharmaceutically acceptable carrier” refers to any ingredient other than the disclosed compounds, or a pharmaceutically acceptable isomer, racemate, hydrate, solvate, homolog or salt thereof (e.g., a carrier capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The formulations can be mixed with auxiliary agents which do not deleteriously react with the active compounds. Such additives can include wetting agents, emulsifying and suspending agents, salt for influencing osmotic pressure, buffers and/or coloring substances, preserving agents, sweetening agents, or flavoring agents. The compositions can also be sterilized if desired.

The route of administration can be any route which effectively transports the active compound of the disclosure to the appropriate or desired site of action, such as oral, nasal, pulmonary, buccal, subdermal, intradermal, transdermal, or parenteral, e.g., rectal, depot, subcutaneous, intravenous, inhalation of a dry powder form or a nebulized form, intraurethral, intramuscular, intranasal, ophthalmic solution, or an ointment, the oral route being preferred.

Dosage forms can be administered once a day, or more than once a day, such as twice or thrice daily. Alternatively, dosage forms can be administered less frequently than daily, such as every other day, or weekly, if found to be advisable by a prescribing physician. Dosing regimens include, for example, dose titration to the extent necessary or useful for the indication to be treated, thus allowing the patient's body to adapt to the treatment and/or to minimize or avoid unwanted side effects associated with the treatment. Other dosage forms include delayed or controlled-release forms. Suitable dosage regimens and/or forms include those set out, for example, in the latest edition of the Physicians' Desk Reference, incorporated herein by reference.

As used herein, the term “administering” or “administration” refers to providing a compound, a pharmaceutical composition comprising the same, to a subject by any acceptable means or route, including (for example) by oral, parenteral (e.g., intravenous), inhaled, or topical administration.

As used herein, the term “treatment” refers to an intervention that ameliorates a sign or symptom of a disease or pathological condition. As used herein, the terms “treatment”, “treat” and “treating,” with reference to a disease, pathological condition or symptom, also refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular disease. A prophylactic treatment is a treatment administered to a subject who does not exhibit signs of a disease or exhibits only early signs, for the purpose of decreasing the risk of developing pathology. A therapeutic treatment is a treatment administered to a subject after signs and symptoms of the disease have developed.

As used herein, the term “subject” refers to an animal (e.g., a mammal, such as a human). A subject to be treated according to the methods described herein may be one who has been diagnosed with a neurodegenerative disease involving demyelination, insufficient myelination, or underdevelopment of a myelin sheath, e.g., a subject diagnosed with multiple sclerosis or cerebral palsy, or one at risk of developing the condition. Diagnosis may be performed by any method or technique known in the art. One skilled in the art will understand that a subject to be treated according to the present disclosure may have been subjected to standard tests or may have been identified, without examination, as one at risk due to the presence of one or more risk factors associated with the disease or condition.

As used herein, the term “effective amount” refers to a quantity of a specified agent sufficient to achieve a desired effect in a subject being treated with that agent. Ideally, an effective amount of an agent is an amount sufficient to inhibit or treat the disease without causing substantial toxicity in the subject. The effective amount of an agent will be dependent on the subject being treated, the severity of the affliction, and the manner of administration of the pharmaceutical composition. Methods of determining an effective amount of the disclosed compound sufficient to achieve a desired effect in a subject will be understood by those of skill in the art in light of this disclosure.

In one embodiment, a method for inhibiting PDGF receptor α is provided, comprising contacting the PDGF receptor α with an effective amount of a compound having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), or a pharmaceutically acceptable isomer, racemate, hydrate, solvate, tautomer, isotope, or salt thereof, or a composition comprising the same.

In one embodiment, a method for inhibiting PDGF receptor β is provided, comprising contacting the PDGF receptor β with an effective amount of a compound having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), or a pharmaceutically acceptable isomer, racemate, hydrate, solvate, tautomer, isotope, or salt thereof, or a composition comprising the same.

In one embodiment, a method for treating a PDGF receptor α-dependent condition is provided, comprising administering to a subject in need thereof an effective amount of a compound having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), or a pharmaceutically acceptable isomer, racemate, hydrate, solvate, tautomer, isotope, or salt thereof, or a composition comprising the same.

In one embodiment, a method for treating a PDGF receptor β-dependent condition, comprising administering to a subject in need thereof an effective amount of a compound having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), or a pharmaceutically acceptable isomer, racemate, hydrate, solvate, tautomer, isotope, or salt thereof, or a composition comprising the same.

In one embodiment, a method for treating a pulmonary disorder is provided, comprising administering to a subject in need thereof an effective amount of a compound having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), or a pharmaceutically acceptable isomer, racemate, hydrate, solvate, tautomer, isotope, or salt thereof, or a composition comprising the same. In one embodiment, the pulmonary disorder is pulmonary hypertension.

In one embodiment, the pulmonary hypertension is pulmonary arterial hypertension. A method for treating pulmonary arterial hypertension is provided, comprising administering to a subject in need thereof an effective amount of a compound having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), or a pharmaceutically acceptable isomer, racemate, hydrate, solvate, tautomer, isotope, or salt thereof, or a composition comprising the same. In one embodiment, the pulmonary arterial hypertension is primary PAH, idiopathic PAH, heritable PAH, refractory PAH, BMPR2, AL 1, endoglin associated with hereditary hemorrhagic telangiectasia, endoglin not associated with hereditary hemorrhagic telangiectasia, drug-induced PAH, or toxin-induced PAH. In another embodiment, the pulmonary arterial hypertension is associated with systemic sclerosis, mixed connective tissue disease, HIV, hepatitis, or portal hypertension.

In one embodiment, the pulmonary hypertension is associated with myeloproliferative disorders. A method for treating pulmonary hypertension associated with myeloproliferative disorders is provided, comprising administering to a subject in need thereof an effective amount of a compound having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), or a pharmaceutically acceptable isomer, racemate, hydrate, solvate, tautomer, isotope, or salt thereof, or a composition comprising the same. In one embodiment, the myeloproliferative disorder associated pulmonary hypertension is Group 5 PAH.

In one embodiment, a method for treating a disease associated with tissue fibrosis, comprising administering to a subject in need thereof an effective amount of a compound having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), or a pharmaceutically acceptable isomer, racemate, hydrate, solvate, tautomer, isotope, or salt thereof, or a composition comprising the same. In one embodiment, the disease associated with tissue fibrosis is systemic sclerosis, interstitial lung disease, bronchiolitis obliterans with organizing pneumonia (BOOP), acute lung injury, glomerulonephritis, focal segmental glomerulosclerosis, stroke, or radiation induced fibrosis.

In one embodiment, a method for solid tumors, comprising administering to a subject in need thereof an effective amount of a compound having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), or a pharmaceutically acceptable isomer, racemate, hydrate, solvate, tautomer, isotope, or salt thereof, or a composition comprising the same. In one embodiment, the solid tumor is associated with an increased copy number of PDGF ligands. In another embodiment, the solid tumor is associated with PDGFRα or PDGFRβ amplification. In another embodiment, the solid tumor is associated with a translocation in the PDGFRα or PDGFRβ kinase domain.

Compounds having the structure of any one of Formulas (I), (II), (III), (IV), (V), (VI), (VII), (VIII), (VIII-A), (VIII-B), (VIII-C), (VIII-D), (VIII-E), (VIII-F), (VIII-G), (VIII-H), (IX), (X), (XI), (XII), (XIII), (XIV), (XV), (XVI), (XVII), (XVIII A), (XVIII B), (XIX), or (XX), can be synthesized using standard synthetic techniques known to those of skill in the art. For examples, compounds of the present disclosure can be synthesized using the general synthetic procedures set forth in Schemes 1-3.

To this end, the reactions, processes and synthetic methods described herein are not limited to the specific conditions described in the following experimental section, but rather are intended as a guide to one with suitable skill in this field. For example, reactions may be carried out in any suitable solvent, or other reagents to perform the transformation[s] necessary. Generally, suitable solvents are protic or aprotic solvents which are substantially non-reactive with the reactants, the intermediates or products at the temperatures at which the reactions are carried out (i.e., temperatures which may range from the freezing to boiling temperatures). A given reaction may be carried out in one solvent or a mixture of more than one solvent. Depending on the particular reaction, suitable solvents for a particular work-up following the reaction may be employed.

Unless otherwise indicated, conventional methods of mass spectroscopy (MS), liquid chromatography-mass spectroscopy (LCMS), NMR, HPLC, protein chemistry, biochemistry, recombinant DNA techniques, and pharmacology are employed. Compounds are prepared using standard organic chemistry techniques such as those described in, for example, March's Advanced Organic Chemistry, 7th Edition, John Wiley and Sons, Inc (2013). Alternate reaction conditions for the synthetic transformations described herein may be employed such as variation of solvent, reaction temperature, reaction time, as well as different chemical reagents and other reaction conditions. As necessary, the use of appropriate protecting groups may be required. The incorporation and cleavage of such groups may be carried out using standard methods described in Peter G. M. Wuts and Theodora W. Green, Protecting Groups in Organic Synthesis, 4th Edition, Wiley-Interscience. (2006). All starting materials and reagents are commercially available or readily prepared.

In some embodiments, arylamide derivatives H1 are synthesized as shown in Scheme 1.

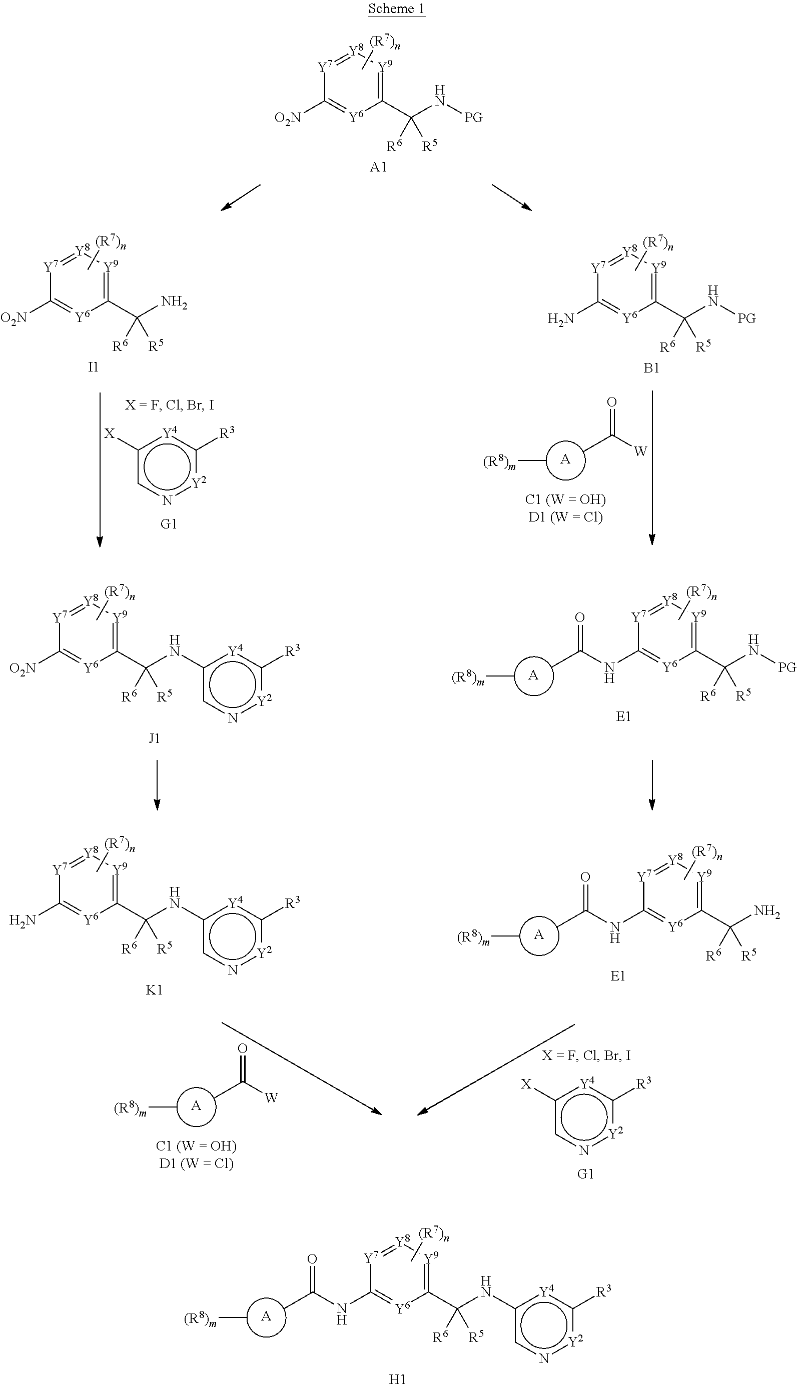

In some embodiments, treatment of suitably N-protected 3-nitro-benzyl derivative A1 with a suitable reducing agent such as hydrogen gas in the presence of Pd/C or Pd(OH)₂/C and in the presence of a suitable solvent such as MeOH, EtOH, or EtOAc will afford the corresponding amino-derivative B1. Alternatively, treatment of A1 with SnCl₂ in the presence of a suitable solvent such as EtOH will afford B1. Treatment of B1 with a carboxylic acid derivative C1 using standard amide coupling conditions will directly afford amide-derivative E1. Alternatively, treatment of carboxylic acid derivative C1 with, for example, SOCl₂, or oxalyl chloride and catalytic DMF, in a suitable solvent such as DCM, or THF will afford acid chloride D1. Subsequent treatment of acid chloride D1 with amino-derivative B1 in the presence of a suitable base such as TEA, Hunig's base, NaHCO₃, or K₂CO₃, and in the presence of a suitable solvent such as DCM or THF, with or without an activating agent such as DMAP, with or without heating will afford amide-derivative E1. Subsequent removal of the N-protecting group (PG) of E1 using appropriate deprotection conditions, will afford amine F1. Treatment of amine F1 with aryl halide G1 (where X=Cl, Br, or I) with heating in the presence of a transition metal catalyst such as Pd[PPh₃]₄, Pd₂(dba)₃, Pd(OAc)₂, Pd(dppf)Cl₂, BrettPhos precatalyst, or CuI, with or without a suitable ligand, such as for example PPh₃, 2-(di-tert-butylphosphino)biphenyl, BrettPhos, BINAP, or trans-N,N-dimethyl-1,2-cyclohexanediamine, and in the presence of a suitable base such as NaO^{t}Bu, Na₂CO₃, or DIEA, and in suitable solvents such as 1,4-dioxane, toluene, THF, acetonitrile, DMF, with or without water, will afford Ill. Alternatively, treatment of F1 with G1 (where X=F, Cl) with or without a base such as K₂CO₃, Cs₂CO₃, TEA, or DIEA, and in a suitable solvent such as THF, DMF, 1,4-dioxane, DMSO, or NMP, with or without heating, will afford Ill. Alternatively, compounds Ill may be prepared from A1 as follows. Removal of the N-protecting group (PG) of A1 using appropriate deprotection conditions, will afford amine I1. Treatment of amine I1 with aryl halide G1 (where X=F, Cl, Br, or I) using the appropriate conditions described above (for the conversion of F1 to H1), will afford J1. Treatment of J1 with a suitable reducing agent such as hydrogen gas in the presence of Pd/C or Pd(OH)₂/C and in the presence of a suitable solvent such as MeOH, EtOH, or EtOAc will afford the corresponding amino-derivative K1. Alternatively, treatment of J1 with SnCl₂ in the presence of a suitable solvent such as EtOH will afford K1. Subsequent treatment of K1 with either carboxylic acid derivative C1, or alternatively, acid chloride derivative D1, using the appropriate conditions described above (for the conversion of B1 to E1), will afford H1.

In some embodiments, arylamide derivatives H2 are synthesized as shown in Scheme 2.

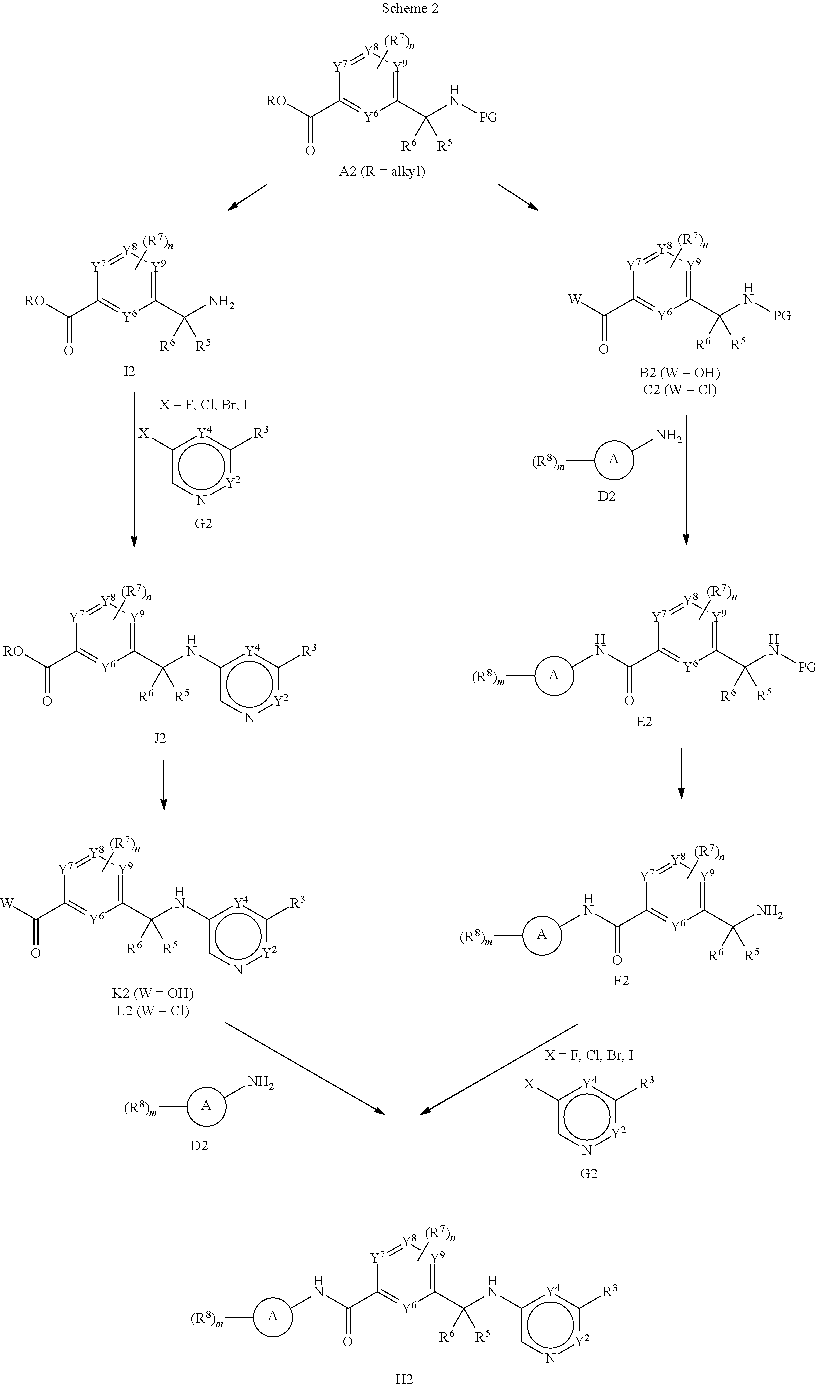

In some embodiments, treatment of suitably N-protected 3-carboxylester-benzyl derivative A2 with a base such as LiOH or NaOH in the presence of water and a suitable solvent such as MeOH or THF, will afford the corresponding acid-derivative B2. Treatment of B2 with an amine D2 using standard amide coupling conditions will directly afford amide-derivative E2. Alternatively, treatment of carboxylic acid derivative B2 with, for example, SOCl₂, or oxalyl chloride and catalytic DMF, in a suitable solvent such as DCM, or THF will afford acid chloride C2. Subsequent treatment of acid chloride C2 with amino-derivative D2 in the presence of a suitable base such as TEA, Hunig's base, NaHCO₃, or K₂CO₃, and in the presence of a suitable solvent such as DCM or THF, with or without an activating agent such as DMAP, with or without heating will afford amide-derivative E2. Subsequent removal of the N-protecting group (PG) of E2 using appropriate deprotection conditions, will afford amine F2. Treatment of amine F2 with aryl halide G2 (where X=Cl, Br, or I) with heating in the presence of a transition metal catalyst such as Pd[PPh₃]₄, Pd₂(dba)₃, Pd(OAc)₂, Pd(dppf)Cl₂, BrettPhos precatalyst, or CuI, with or without a suitable ligand, such as for example PPh₃, 2-(di-tert-butylphosphino)biphenyl, BrettPhos, BINAP, or trans-N,N-dimethyl-1,2-cyclohexanediamine, and in the presence of a suitable base such as NaO^{t}Bu, Na₂CO₃, or DIEA, and in suitable solvents such as 1,4-dioxane, toluene, THF, acetonitrile, DMF, with or without water, will afford H2. Alternatively, treatment of F2 with G2 (where X=F, Cl) with or without a base such as K₂CO₃, Cs₂CO₃, TEA, or DIEA, and in a suitable solvent such as THF, DMF, 1,4-dioxane, DMSO, or NMP, with or without heating, will afford H2. Alternatively, compounds H2 may be prepared from A2 as follows. Removal of the N-protecting group (PG) of A2 using appropriate deprotection conditions, will afford amine 12. Treatment of amine 12 with aryl halide G2 (where X=F, Cl, Br, or I) using the appropriate conditions described above (for the conversion of F2 to H2), will afford J2. Treatment of J2 with a base such as LiOH or NaOH in the presence of water and a suitable solvent such as MeOH or THF, will afford the corresponding acid-derivative K2. Conversion of K2 to H2, either directly or via acid chloride L2, may be achieved using the appropriate conditions described above (for the conversion of B2 to E2).

In some embodiments, arylurea derivatives J3 are synthesized as shown in Scheme 3.

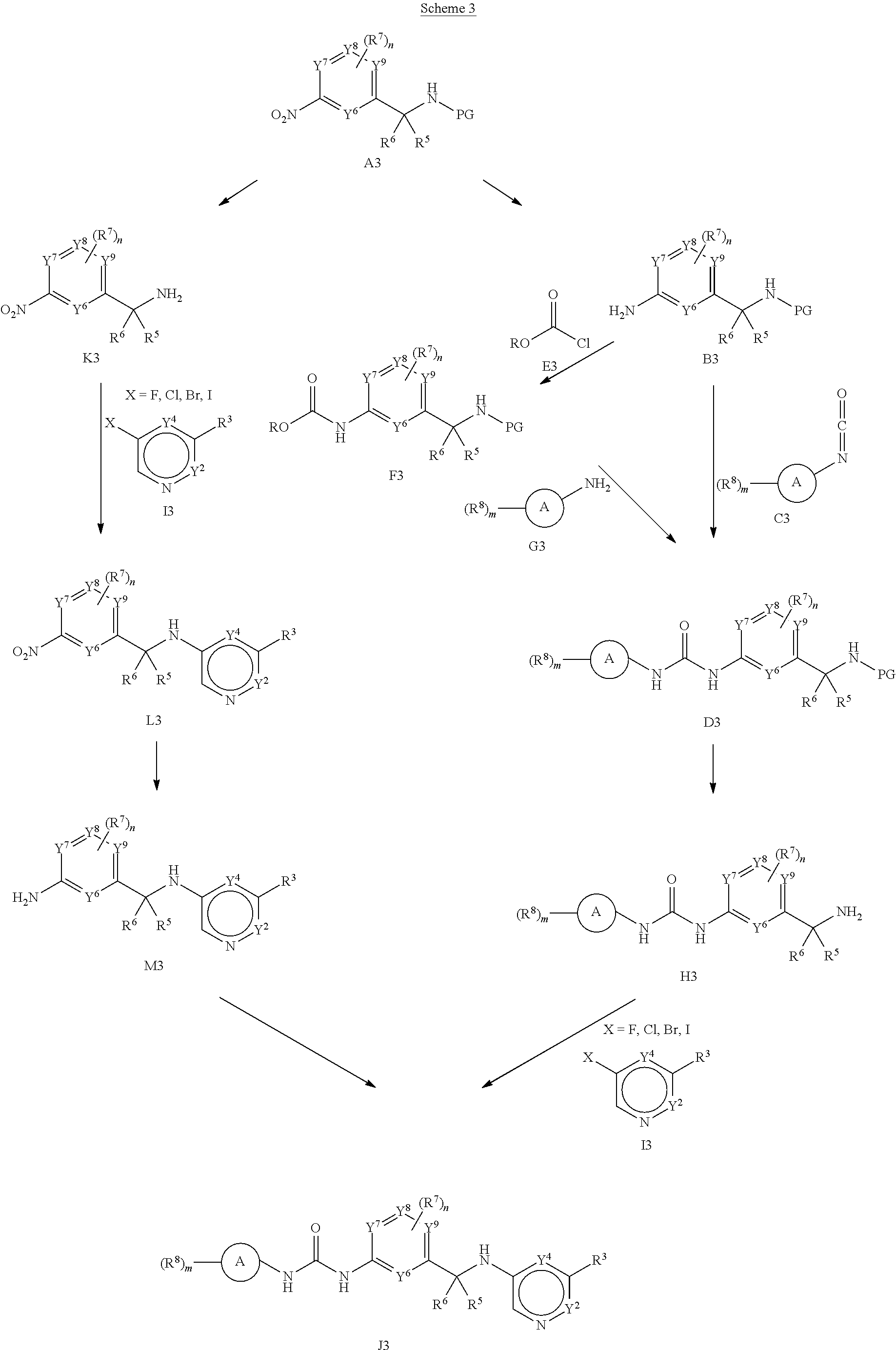

In some embodiments, treatment of suitably N-protected 3-nitro-benzyl derivative A3 with a suitable reducing agent such as hydrogen gas in the presence of Pd/C or Pd(OH)₂/C and in the presence of a suitable solvent such as MeOH, EtOH, or EtOAc will afford the corresponding amino-derivative B3. Alternatively, treatment of A3 with SnCl₂ in the presence of a suitable solvent such as EtOH will afford B3. Treatment of B3 with an isocyanate derivative C3 in the presence of a suitable solvent such as DCM, THF, or DMF, with or without heating, will directly afford urea-derivative D3. Alternatively, treatment of amine B3 with a suitable substituted chloroformate derivative E3 (where R can be for example 4-nitrophenyl or isopropenyl) in the presence of a suitable base such as TEA, DIEA, or NaHCO₃, and in a suitable solvent such as DCM, EtOAc, or THF, will afford intermediate carbamate F3. Subsequent treatment of carbamate F3 with an amine G3 in a suitable solvent such as DCM, THF, or 1,4-dioxane, with or without a base such as TEA, DIEA, or DMAP, and with or without heating, will afford D3. Subsequent removal of the N-protecting group (PG) of D3 using appropriate deprotection conditions will afford amine H3. Treatment of amine H3 with aryl halide 13 (where X=Cl, Br, or I) with heating in the presence of a transition metal catalyst such as Pd[PPh₃]₄, Pd₂(dba)₃, Pd(OAc)₂, Pd(dppf)Cl₂, BrettPhos precatalyst, or CuI, with or without a suitable ligand, such as for example PPh₃, 2-(di-tert-butylphosphino)biphenyl, BrettPhos, BINAP, or trans-N,N-dimethyl-1,2-cyclohexanediamine, and in the presence of a suitable base such as NaO^{t}Bu, Na₂CO₃, or DIEA, and in suitable solvents such as 1,4-dioxane, toluene, THF, acetonitrile, DMF, with or without water, will afford J3. Alternatively, treatment of H3 with I3 (where X=F, Cl) with or without a base such as K₂CO₃, Cs₂CO₃, TEA, or DIEA, and in a suitable solvent such as THF, DMF, 1,4-dioxane, DMSO, or NMP, with or without heating, will afford J3. Alternatively, compounds J3 may be prepared from A3 as follows. Removal of the N-protecting group (PG) of A3 using appropriate deprotection conditions, will afford amine K3. Treatment of amine K3 with aryl halide I3 (where X=F, Cl, Br, or I) using the appropriate conditions described above (for the conversion of H3 to J3), will afford L3. Treatment of L3 with a suitable reducing agent (as described above for the conversion of A3 to B3) will afford the corresponding amine M3. Conversion of M3 to J3, may be achieved using the appropriate conditions described above (for the conversion of B3 to D3).

### EXAMPLES

The invention is further illustrated by the following examples. The examples below are non-limiting are merely representative of various aspects of the invention. Solid and dotted wedges within the structures herein disclosed illustrate relative stereochemistry, with absolute stereochemistry depicted only when specifically stated or delineated. The following examples were prepared according to the methods described in Schemes 1 through 3 using the appropriately substituted or modified intermediates.

### Example 1

### Synthesis of the hydrochloride salt of (5)-5-Methyl-N-(3-(1-((5-methyl-5H-pyrrolo[2,3-b] pyrazin-3-yl)amino)ethyl)phenyl)nicotinamide (Compound 1)

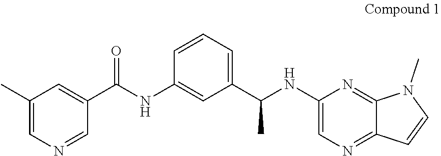 [SMILES: Cc1cncc(C(=O)Nc2cccc([C@H](C)Nc3cnc4ccn(C)c4n3)c2)c1]

### Step 1: Synthesis of (S)-1-(1-azidoethyl)-3-nitrobenzene (1-b)

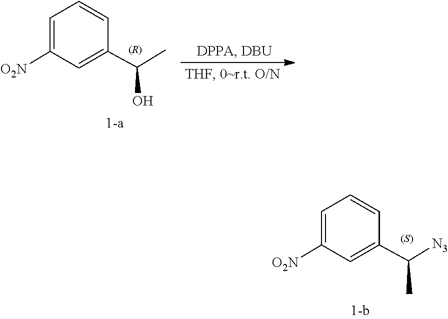

To a stirred solution of (R)-1-(3-nitrophenyl)ethan-1-ol (1-a) (10.00 g, 0.059 mol) in THF at 0° C., was added DPPA (19.76 g, 0.072 mol). After 5 min, DBU (27.32 g, 0.179 mol) was added dropwise. The mixture was stirred at 20° C. for 16 h. The mixture was concentrated and the crude residue purified (silica gel; eluting with 2% EtOAc in petroleum ether) to afford compound 1-b (9.22 g, 80%) as a yellow oil. ¹H NMR (400 MHz, DMSO-d₆) δ 8.35-8.12 (m, 2H), 7.88 (d, J=7.7 Hz, 1H), 7.72 (t, J=7.9 Hz, 1H), 5.10 (q, J=6.8 Hz, 1H), 1.53 (d, J=6.8 Hz, 3H).

### Step 2: Synthesis of (S)-1-(3-nitrophenyl)ethan-1-amine (1-c)

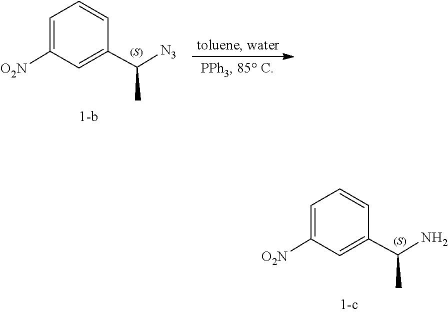

To a mixture of compound (1-b) (9.22 g, 0.048 mol) in toluene (100 mL), was added water (30 mL) and PPh₃ (25.17 g, 0.095 mol). The mixture was stirred at 85° C. for 5 h. After cooling to rt, the mixture was diluted with aq. HCl (3N, 500 mL) and washed with EtOAc (500 mL×3). The aqueous layer was cooled to 0° C., and the pH was adjusted to 12 with aq. 30% NaOH. The aqueous layer was extracted with DCM (500 mL×3), and the combined organic phase dried (Na₂SO₄), filtered, and concentrated under reduced pressure to afford compound 1-c (7.25 g, 91%) as a yellow oil. ¹H NMR (400 MHz, DMSO-d₆) δ 8.28 (s, 1H), 8.07 (dd, J=8.2, 2.3 Hz, 1H), 7.83 (d, J=7.7 Hz, 1H), 7.60 (t, J=7.9 Hz, 1H), 4.17 (q, J=6.6 Hz, 1H), 1.29 (d, J=6.6 Hz, 3H). LCMS Mass: 167.1 (M⁺+H).

### Step 3: Synthesis of tert-butyl (S)-(1-(3-nitrophenyl)ethyl)carbamate (1-d)

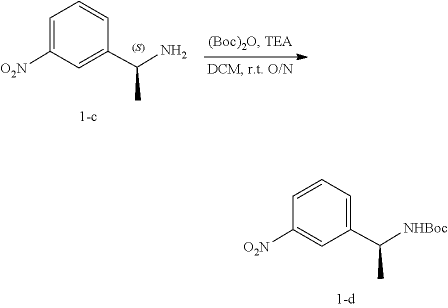

To a stirred solution of compound (1-c) (3.00 g, 18.1 mmol) in DCM (40 mL), was added TEA (3.64 g, 36.0 mmol) and (Boc)₂O (5.89 g, 27.0 mmol). The mixture was stirred at rt for 16 h. The mixture was concentrated under reduced pressure and partitioned between water (100 mL) and EtOAc (80 mL). The organic layer was separated, dried (Na₂SO₄), filtered, and was concentrated under reduced pressure. The crude residue was purified (silica gel; eluting with 0-10% EtOAc in petroleum ether) to afford compound 1-cl (3.90 g, 81%) as an off-white solid. ¹H NMR (400 MHz, CDCl₃): δ 8.17 (s, 1H), 8.11 (d, J=8.1 Hz, 1H), 7.64 (d, J=7.6 Hz, 1H), 7.50 (t, J=7.9 Hz, 1H), 4.88 (s, 2H), 1.47 (d, J=6.6 Hz, 3H), 1.42 (s, 9H); LCMS Mass: 211.1 (MH⁺−56) and 167.1 (MH⁺-100).

### Step 4: Synthesis of tert-butyl (S)-(1-(3-aminophenyl)ethyl)carbamate (1-e)

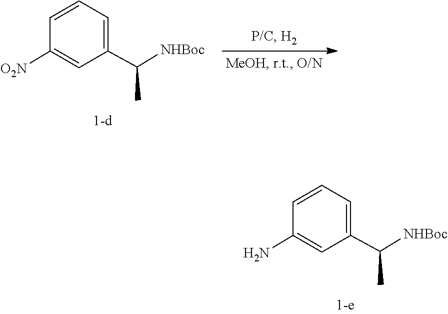 [SMILES: C[C@H](NC(=O)OC(C)(C)C)c1cccc([N+](=O)[O-])c1 ; C[C@H](NC(=O)OC(C)(C)C)c1cccc(N)c1]

To a stirred solution of compound (1-d) (3.90 g, 14.6 mmol) in methanol (50 mL), was added Pd/C (400 mg). The mixture was stirred at rt for 16 h under H₂ (1 atmosphere pressure). The reaction mixture was filtered through Celite, and the solid residue was washed with methanol (20 mL). The filtrate was concentrated to dryness under reduced pressure to afford compound 1-e (3.40 g, 98%) as a yellow solid. ¹H NMR (400 MHz, CDCl₃): δ 7.13 (t, J=7.7 Hz, 1H), 6.79-6.69 (m, 2H), 6.69-6.59 (m, 1H), 4.80 (s, 1H), 4.69 (s, 1H), 4.20-3.25 (brs, 2H), 1.41 (s, 12H); LCMS Mass: 181.1 (MH⁺−56).

### Step 5: Synthesis of tert-butyl (S)-(1-(3-(5-methylnicotinamido)phenyl)ethyl)carbamate (1-f)

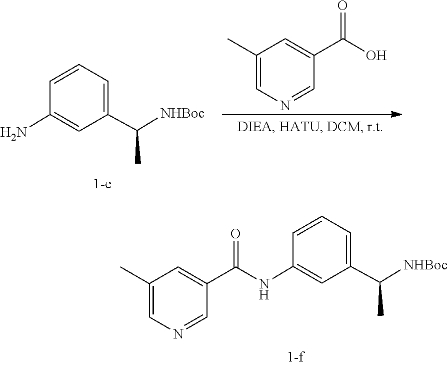

To a stirred solution of 5-methylnicotinic acid (3.21 g, 23.4 mmol) in DMF (50 mL) at rt, was added HATU (11.90 g, 31.2 mmol) and the mixture was stirred at rt for 20 min. Compound (1-e) (3.40 g, 15.6 mmol) and DIPEA (6.05 g, 46.8 mmol) were added and the mixture stirred at rt for 16 h. The reaction mixture was partitioned between water (200 mL) and EtOAc (120 mL). The organic layer was separated and washed with brine (100 mL×2). The organic layer was separated, dried (Na₂SO₄), filtered, and was concentrated under reduced pressure. The crude residue was purified (silica gel; eluting with 0-50% EtOAc in petroleum ether) to afford compound 1-f (4.80 g, 92%) as an off-white solid. ¹H NMR (400 MHz, CDCl₃): δ 8.98 (s, 1H), 8.56 (s, 1H), 8.48 (brs, 1H), 8.13 (s, 1H), 7.60-7.57 (m, 2H), 7.31 (t, J=7.8 Hz, 1H), 7.10 (d, J=7.6 Hz, 1H), 4.95 (d, J=5.6 Hz, 1H), 4.75 (s, 1H), 2.43 (s, 3H), 1.45 (d, J=7.0 Hz, 3H), 1.41 (s, 9H); LCMS Mass: 356.1 (M⁺+H).

### Step 6: Synthesis of (S)—N-(3-(1-aminoethyl)phenyl)-5-methylnicotinamide (1-g)

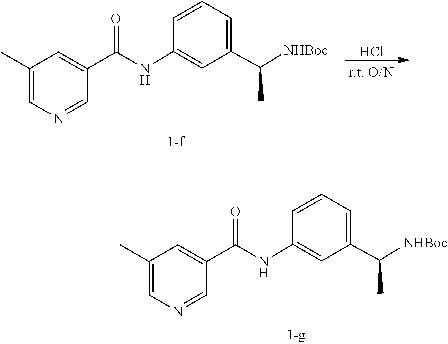 [SMILES: Cc1cncc(C(=O)Nc2cccc([C@H](C)NC(=O)OC(C)(C)C)c2)c1 ; Cl ; Cc1cncc(C(=O)Nc2cccc([C@H](C)NC(=O)OC(C)(C)C)c2)c1]

To a stirred solution of compound (1-f) (4.80 g, 13.5 mmol) in DCM (40 mL), was added 4 M HCl in 1,4-dioxane (4 mL) and the mixture was stirred at rt for 16 h. The mixture was evaporated under reduced pressure and the crude residue was adjusted to pH 8 with saturated aq. Na₂CO₃. The mixture was dissolved in methanol and purified (C-18 reverse-phase column chromatography) to afford compound 1-g (2.40 g, 70%) as a yellow oil. ¹H NMR (400 MHz, DMSO-d₆): δ 10.36 (s, 1H), 8.91 (d, J=1.9 Hz, 1H), 8.60 (d, J=1.6 Hz, 1H), 8.12 (s, 1H), 7.75 (s, 1H), 7.64 (d, J=8.0 Hz, 1H), 7.28 (t, J=7.8 Hz, 1H), 7.13 (d, J=7.7 Hz, 1H), 4.01 (q, J=6.6 Hz, 1H), 2.39 (s, 3H), 1.27 (d, J=6.6 Hz, 3H); LCMS Mass: 256.1 (M⁺+H).

### Step 7: Synthesis of 3-chloro-5-methyl-5H-pyrrolo[2,3-b]pyrazine (1-i)

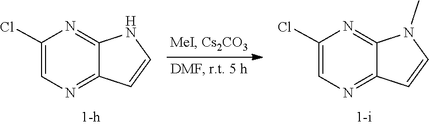

To a stirred solution of 3-chloro-5H-pyrrolo[2,3-b]pyrazine (1-h) (50 mg, 0.326 mmol) in DMF (3 mL) was added methyl iodide (231 mg, 1.628 mmol) and Cs₂CO₃ (212 mg, 0.651 mmol). The mixture was stirred at rt for 1 h. The mixture was evaporated under reduced pressure. The resulting mixture was partitioned between water (50 mL) and EtOAc (50 mL). The organic layer was separated, dried (Na₂SO₄), filtered, and was concentrated under reduced pressure. The crude residue was purified (C-18 reverse-phase column; eluting with 60% MeOH in water) to afford compound 1-i (23 mg, 42.0%) as an off white solid. LCMS Mass: 168.1 (M⁺+H).

### Step 8: Synthesis of the hydrochloride salt of (S)-5-methyl-N-(3-(1-((5-methyl-5H-pyrrolo[2,3-b]pyrazin-3-yl)amino)ethyl)phenyl)nicotinamide (Compound 1)

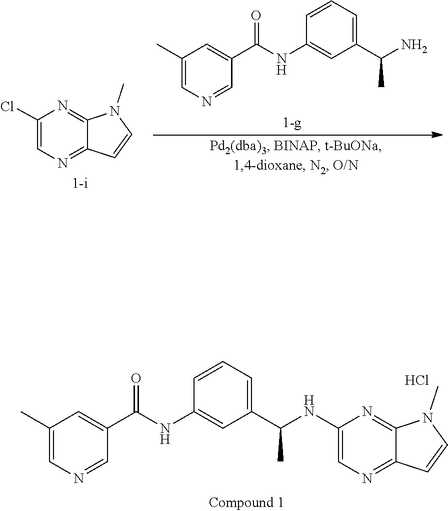 [SMILES: Cn1ccc2ncc(Cl)nc21 ; Cc1cncc(C(=O)Nc2cccc([C@H](C)N)c2)c1 ; Cc1cncc(C(=O)Nc2cccc([C@H](C)Nc3cnc4ccn(C)c4n3)c2)c1 ; Cl]

To a stirred solution of compound (1-i) (23 mg, 0.137 mmol) in 1,4-dioxane (3 mL) at rt, was added (S)—N-(3-(1-aminoethyl)phenyl)-5-methylnicotinamide (1-g) (38 mg, 0.151 mmol), Pd₂(dba)₃ (13 mg, 0.014 mmol), BINAP (9 mg, 0.014 mmol) and t-BuONa (26 mg, 0.274 mmol). The mixture was heated to reflux under N₂ for 16 h. The reaction mixture was partitioned between brine (50 mL) and EtOAc (50 mL). The organic layer was separated, dried (Na₂SO₄), filtered, and was concentrated under reduced pressure. The crude residue was purified (Preparative HPLC; eluting with 0.1% HCl in H₂O/MeOH) to afford the hydrochloride salt of Compound 1 (18 mg, 34%). ¹H NMR (400 MHz, MeOH-d₄): δ 9.19 (s, 1H), 8.95 (s, 1H), 8.88 (s, 1H), 7.97 (s, 1H), 7.87 (s, 1H), 7.62-7.57 (m, 2H), 7.40-7.29 (m, 2H), 6.54 (d, J=3.7 Hz, 1H), 5.18 (q, J=6.9 Hz, 1H), 3.76 (s, 3H), 2.65 (s, 3H), 1.65 (d, J=6.9 Hz, 3H); LCMS Mass: 387.2 (M⁺+H).

### Example 2

### Synthesis of (S)-5-methyl-N-(3-(1-(quinolin-3-ylamino)ethyl)phenyl)nicotinamide (Compound 2)

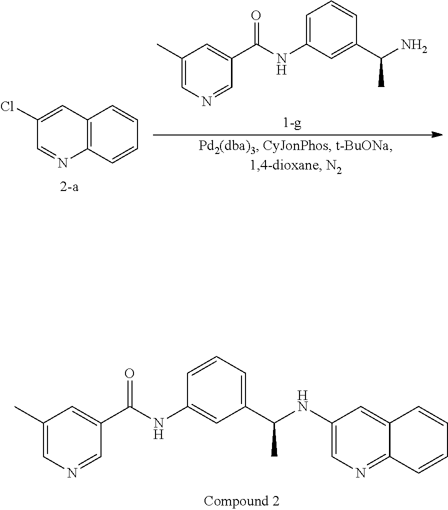 [SMILES: Cc1cncc(C(=O)Nc2cccc([C@H](C)N)c2)c1 ; Clc1cnc2ccccc2c1 ; Cc1cncc(C(=O)Nc2cccc([C@H](C)Nc3cnc4ccccc4c3)c2)c1]

To a stirred solution of 3-bromoquinoline (2-a) (25 mg, 0.120 mmol) in 1,4-dioxane (3 mL) at rt, was added (S)—N-(3-(1-aminoethyl)phenyl)-5-methylnicotinamide (1-g) (prepared as described in Example 1, Step 6) (34 mg, 0.132 mmol), Pd₂(dba)₃ (9.7 mg, 0.012 mmol), CyJohnPhos (4.2 mg, 0.012 mmol) and t-BuONa (2M, 90 μL). The mixture was heated to 100° C. under N₂ for 30 min. The reaction mixture was filtered and purified (Preparative HPLC; eluting with 0.1% TFA in H₂O/acetonitrile). The combined fractions were diluted with EtOAc and washed with saturated aq. NaHCO₃ and brine. The organic phase was dried (MgSO₄), filtered, and concentrated under reduced pressure to afford compound Compound 2 (32 mg, 66%). LCMS Mass: 383.2 (M⁺+H).

### Example 3

### Synthesis of (S)—N-(3-(1-((1,5-naphthyridin-3-yl)amino)ethyl)phenyl)-5-methylnicotinamide (Compound 3)

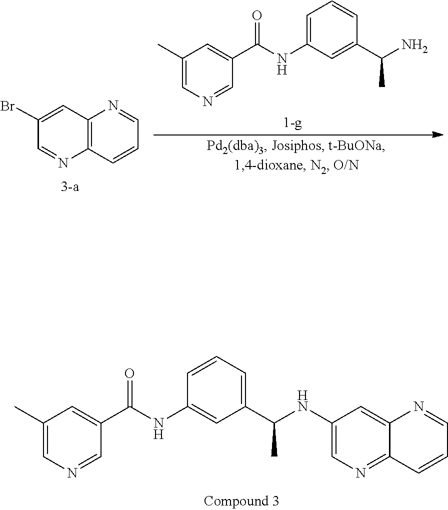 [SMILES: Cc1cncc(C(=O)Nc2cccc([C@H](C)N)c2)c1 ; Brc1cnc2cccnc2c1 ; Cc1cncc(C(=O)Nc2cccc([C@H](C)Nc3cnc4cccnc4c3)c2)c1]

To a stirred solution of 3-bromo-1,5-naphthyridine (3-a) (25 mg, 0.120 mmol) in 1,4-dioxane (1 mL) at rt, was added (S)—N-(3-(1-aminoethyl)phenyl)-5-methylnicotinamide (1-g) (prepared as described in Example 1, Step 6) (34 mg, 0.132 mmol), Pd₂(dba)₃ (11 mg, 0.012 mmol), Josiphos (8 mg, 0.012 mmol) and t-BuONa (2M, 120 μL). The mixture was heated to reflux under N₂ for 16 h. The reaction mixture was filtered and purified (Preparative HPLC; eluting with 0.05% TFA in H₂O/acetonitrile). The combined fractions were diluted with EtOAc and washed with saturated aq. NaHCO₃ and brine. The organic phase dried (MgSO₄), filtered, and concentrated under reduced pressure to afford Compound 3 (7 mg, 15%). ¹H NMR (MeOH-d4, 300 MHz) δ 8.84 (d, 1H, J=1.7 Hz), 8.62 (d, 1H, J=2.7 Hz), 8.59 (dd, 1H, J=1.6, 4.4 Hz), 8.54 (d, 1H, J=1.4 Hz), 8.17 (dd, 1H, J=0.8, 8.3 Hz), 8.14 (s, 1H), 7.82 (s, 1H), 7.6-7.6 (m, 1H), 7.3-7.4 (m, 2H), 7.2-7.3 (m, 1H), 6.92 (d, 1H, J=2.6 Hz), 4.62 (q, 1H, J=6.8 Hz), 2.43 (s, 3H), 1.64 (d, 3H, J=6.8 Hz); LCMS Mass: 384.2 (M⁺+H).

### Example 4

### Synthesis of (5)-5-methyl-N-(3-(1-(quinoxalin-2-ylamino)ethyl)phenyl)nicotinamide trifluoroacetate (Compound 4)

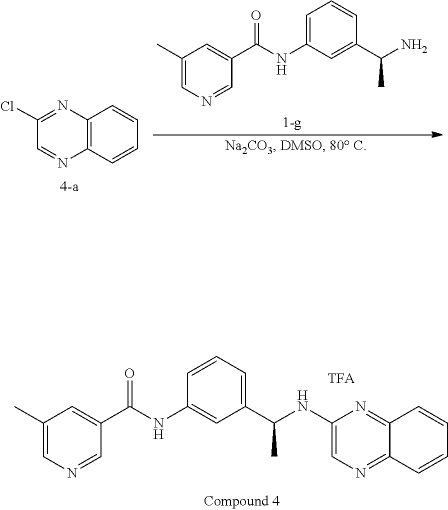 [SMILES: Cc1cncc(C(=O)Nc2cccc([C@H](C)N)c2)c1 ; Clc1cnc2ccccc2n1 ; Cc1cncc(C(=O)Nc2cccc([C@H](C)Nc3cnc4ccccc4n3)c2)c1]

To a stirred solution of 2-chloroquinoxaline (4-a) (25 mg, 0.152 mmol) in DMSO (1 mL) was added (S)—N-(3-(1-aminoethyl)phenyl)-5-methylnicotinamide (1-g) (prepared as described in Example 1, Step 6) (78 mg, 0.304 mmol). The reaction mixture was stirred at rt overnight and K₂CO₃ (31 mg, 0.228 mmol) was added. The reaction was heated to 80° C. for 6 h. The mixture was purified (Preparative HPLC; eluting with 0.1% TFA in H₂O/acetonitrile) to afford Compound 4 (8 mg, 10%). ¹H NMR (MeOH-d4, 300 MHz) δ 9.0-9.0 (m, 1H), 8.70 (d, 1H, J=1.2 Hz), 8.4-8.5 (m, 2H), 7.9-8.0 (m, 1H), 7.9-7.9 (m, 1H), 7.6-7.7 (m, 2H), 7.6-7.6 (m, 1H), 7.4-7.5 (m, 2H), 7.3-7.4 (m, 1H), 5.3-5.4 (m, 1H), 2.55 (s, 3H), 1.72 (d, 3H, J=6.8 Hz); LCMS Mass: 384.2 (M⁺+H).

### Example 5

### Synthesis of (S)-5-methyl-N-(3-(1-(pyrido[2,3-b]pyrazin-3-ylamino)ethyl)phenyl) nicotinamide (Compound 5)

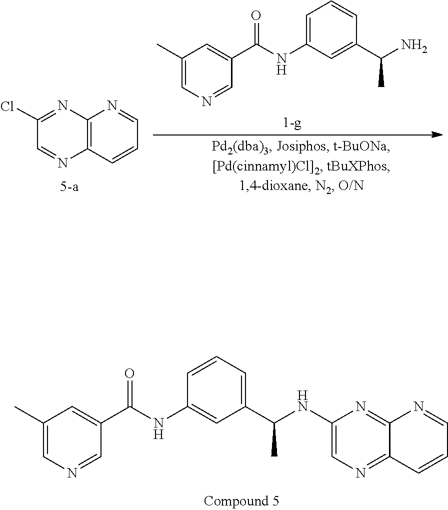 [SMILES: Cc1cncc(C(=O)Nc2cccc([C@H](C)N)c2)c1 ; Clc1cnc2cccnc2n1 ; Cc1cncc(C(=O)Nc2cccc([C@H](C)Nc3cnc4cccnc4n3)c2)c1]

To a stirred solution of 3-chloropyrido[2,3-b]pyrazine (5-a) (25 mg, 0.151 mmol) in 1,4-dioxane (1 mL) at rt, was added (S)—N-(3-(1-aminoethyl)phenyl)-5-methylnicotinamide (1-g) (prepared as described in Example 1, Step 6) (42 mg, 0.166 mmol), Pd₂(dba)₃ (14 mg, 0.015 mmol), Josiphos (9.7 mg, 0.015 mmol) and t-BuONa (2M, 151 μL). The mixture was heated to reflux under N₂ for 4 h. Additional Pd₂(dba)₃ (14 mg, 0.015 mmol), Josiphos (9.7 mg, 0.015 mmol) and t-BuONa (2M, 75 μL) were added to the reaction mixture and heating was continued for 16 h. The reaction mixture was filtered and purified (Preparative HPLC; eluting with 0.05% TFA in H₂O/acetonitrile). The material isolated was combined with batch 2 below.

To a second stirred solution of 3-chloropyrido[2,3-b]pyrazine (5-a) (40 mg, 0.241 mmol) in 1,4-dioxane (1 mL) at rt, was added (S)—N-(3-(1-aminoethyl)phenyl)-5-methylnicotinamide (1-g) (prepared as described in Example 1, Step 6) (42 mg, 0.166 mmol), Pd₂(dba)₃ (14 mg, 0.015 mmol), Josiphos (9.7 mg, 0.015 mmol) and t-BuONa (2M, 226 μL). The mixture was heated to reflux under N₂ for 2 h. [Pd(cinnamyl)Cl]₂ (7.8 mg, 0.015 mmol), tBuXPhos (6.4 mg, 0.015 mmol), and t-BuONa (2 M, 226 μL) were added and the reaction was heated at 100° C. for 16 h. The reaction mixture was filtered and purified (Preparative HPLC; eluting with 0.05% TFA in H₂O/acetonitrile). The combined fractions from both batches were diluted with EtOAc and washed with saturated aq. NaHCO₃ and brine. The organic phases were dried (MgSO₄), filtered, and concentrated under reduced pressure. The crude residue was purified (silica gel; eluting with 0-100% EtOAc in heptane followed by 10-30% MeOH in DCM) to afford Compound 5 (6 mg, 7%). ¹H NMR (MeOH-d4, 300 MHz) δ 8.85 (s, 1H), 8.5-8.6 (m, 2H), 8.48 (s, 1H), 8.15 (s, 1H), 8.00 (dd, 1H, J=1.7, 8.3 Hz), 7.84 (s, 1H), 7.5-7.6 (m, 2H), 7.2-7.4 (m, 2H), 5.33 (q, 1H, J=6.9 Hz), 2.44 (s, 3H), 1.63 (d, 3H, J=7.0 Hz); LCMS Mass: 385.20 (M⁺+H).

### Example 6

### Synthesis of the hydrochloride salt of (S)—N-(3-(1-((1-(3,4-dimethoxyphenyl)-1H-pyrrolo [3,2-b]pyridin-6-yl)amino)ethyl)phenyl)-5-methylnicotinamide (Compound 6)

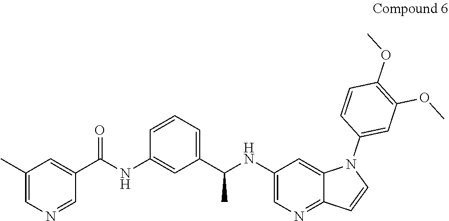

### Step 1: Synthesis of 6-bromo-1-(3,4-dimethoxyphenyl)-1H-pyrrolo[3,2-b]pyridine (6-b)

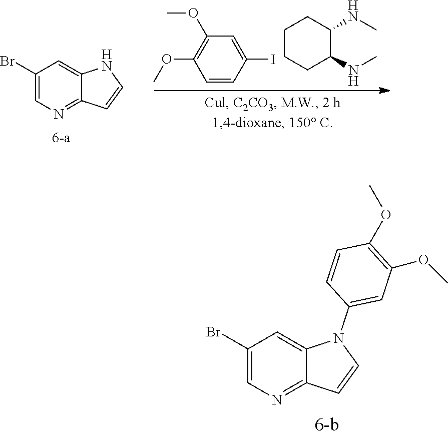

To a stirred solution of 6-bromo-1H-pyrrolo[3,2-b]pyridine (6-a) (400 mg, 2.030 mol) in 1,4-dioxane (8 mL) was added 4-iodo-1,2-dimethoxybenzene (536 mg, 2.030 mol), (1S,2S)—N¹,N²-dimethylcyclohexane-1,2-diamine (58 mg, 0.406 mmol), CuI (39 mg, 0.203 mmol) and Cs₂CO₃ (1.32 g, 4.060 mmol). The mixture was placed in a microwave and heated at 150° C. for 2 h. The mixture was concentrated and purified (silica gel; eluting with EtOAc/PE=1/50-1/10) to afford compound 6-b (206 mg, 31%) as an off-white solid. ¹H NMR (400 MHz, DMSO-d₆) δ 8.63-8.40 (m, 1H), 8.22-8.02 (m, 1H), 7.94 (dd, J=26.8, 3.3 Hz, 1H), 7.14 (d, J=10.3 Hz, 3H), 6.86-6.74 (m, 1H), 3.83 (s, 6H); LCMS Mass: 333.0 (M⁺+H).

### Step 2: Synthesis the hydrochloride salt of (S)—N-(3-(1-((1-(3,4-dimethoxyphenyl)-1H-pyrrolo[3,2-b]pyridin-6-yl)amino)ethyl)phenyl)-5-methylnicotinamide (Compound 6)

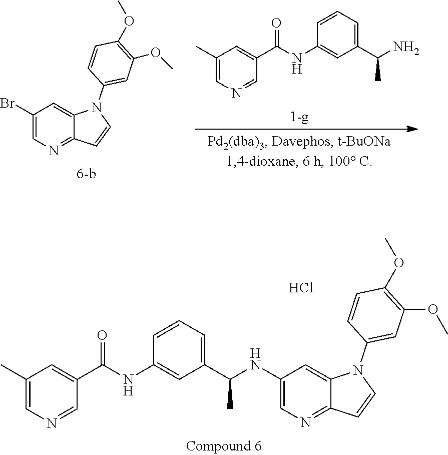

To a mixture of compound (6-b) (200 mg, 0.600 mmol) in 1,4-dioxane (5 mL) was added Pd₂(dba)₃ (55 mg, 0.060 mmol), (S)—N-(3-(1-aminoethyl)phenyl)-5-methylnicotinamide (1-g) (prepared as described in Example 1, Step 6) (153 mg, 0.600 mmol), Davephos (24 mg, 0.060 mmol) and t-BuONa (173 mg, 1.801 mmol). The mixture was stirred at 100° C. for 6 h under nitrogen atmosphere. The mixture was cooled to rt and concentrated and the residue was purified (reverse-phase HPLC; eluting with MeOH/water/HCl) to afford the hydrochloride salt of Compound 6 (8 mg) as a yellow solid. ¹H NMR (400 MHz, CD₃OD) δ 9.12 (s, 1H), 8.91 (s, 1H), 8.82 (s, 1H), 7.91 (s, 1H), 7.81 (s, 1H), 7.73 (d, J=3.4 Hz, 1H), 7.52 (d, J=5.6 Hz, 1H), 7.34 (s, 1H), 7.28 (t, J=7.9 Hz, 1H), 7.14 (d, J=7.7 Hz, 1H), 6.98 (d, J=8.5 Hz, 1H), 6.87 (d, J=2.1 Hz, 1H), 6.82-6.73 (m, 1H), 6.65 (d, J=3.3 Hz, 1H), 4.45 (q, J=6.6 Hz, 1H), 3.74 (s, 3H), 3.70 (s, 3H), 2.58 (s, 3H), 1.50 (d, J=6.7 Hz, 3H); LCMS Mass: 508.1 (M⁺+H).

### Example 7

### Synthesis of the hydrochloride salt of (S)—N-(3-(1-((1-ethyl-1H-pyrazolo[4,3-b]pyridin-6-yl)ethyl)phenyl)-5-methylnicotinamide (Compound 7)

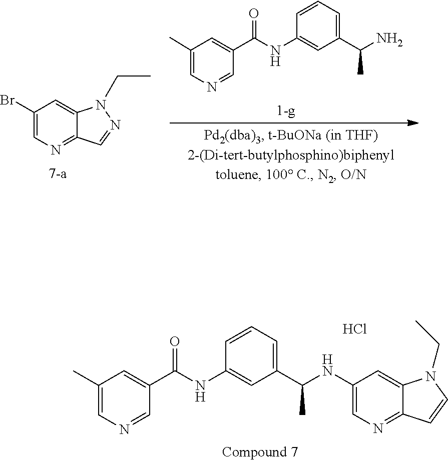

To a stirred solution of 6-bromo-1-ethyl-1H-pyrazolo[4,3-b]pyridine (7-a) (80 mg, 0.35 mmol) and (S)—N-(3-(1-aminoethyl)phenyl)-5-methylnicotinamide (1-g) (prepared as described in Example 1, Step 6) (100 mg, 0.4 mmol) in toluene was added Pd₂(dba)₃ (32.05 mg, 0.035 mmol), t-BuONa (84 mg, 0.875 mmol) and 2-(di-tert-butylphosphino)biphenyl (10.5 mg, 0.035 mmol) and the mixture was stirred at 100° C. under N₂ atmosphere overnight. The mixture was extracted with DCM (20 mL×3). The organic phase was combined and dried (Na₂SO₄), filtered, and was concentrated under reduced pressure. The crude was purified (Reverse-Phase Preparative HPLC; eluting with 0.1% HCl in H₂O/MeOH) to afford afford the hydrochloride salt of Compound 7 (50 mg, 36%). ¹H NMR (400 MHz, CD₃OD) δ 9.21-9.15 (m, 1H), 8.93 (s, 1H), 8.87 (s, 1H), 8.37-8.32 (m, 1H), 8.11 (s, 1H), 8.00 (d, J=27.2 Hz, 1H), 7.53 (s, 1H), 7.39-7.35 (m, 2H), 7.33 (d, J=5.4 Hz, 1H), 4.74 (s, 1H), 4.42-4.34 (m, 2H), 2.61 (s, 3H), 1.63 (s, 3H), 1.33 (s, 3H); LCMS Mass: 401.3 (M⁺+H).

### Example 8

### Synthesis of the hydrochloride salt of (S)-5-methyl-N-(3-(1-((1-methyl-1H-pyrazolo[3,4-b]pyrazin-6-yl)amino)ethyl)phenyl)nicotinamide (Compound 8)

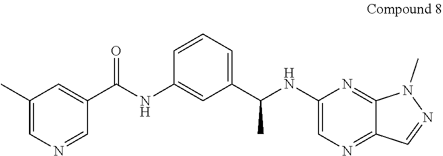 [SMILES: Cc1cncc(C(=O)Nc2cccc([C@H](C)Nc3cnc4cnn(C)c4n3)c2)c1]

### Step 1: Synthesis of 6-chloro-1-methyl-1H-pyrazolo[3,4-b]pyrazine (8-b)

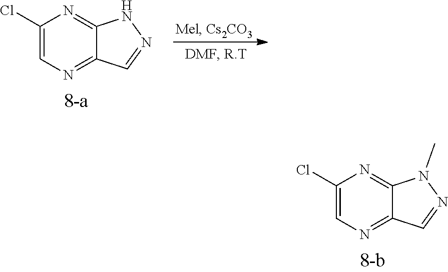

To a stirred solution of 6-chloro-1H-pyrazolo[3,4-b]pyrazine (8-a) (100 mg, 0.64 mmol) in DMF (10 mL), was added Cs₂CO₃ (421 mg, 1.29 mmol) and iodomethane (459 mg, 3.23 mmol). The mixture was stirred at r.t for 2 h. The mixture was evaporated under reduced pressure. The reaction mixture was partitioned between water (50 mL) and EtOAc (50 mL). The organic layer was separated, dried (Na₂SO₄), filtered, and concentrated under reduced pressure. The crude residue was purified (silica gel; eluting with 0-30% EtOAc in PE), to afford compound 8-b (56 mg, 51%) as an off white solid. ¹H NMR (400 MHz, CDCl₃): δ 8.53 (s, 1H), 8.27 (s, 1H), 4.14 (s, 3H); LCMS Mass: 169.0 (M⁺+H).

### Step 2: Synthesis of the hydrochloride salt of (S)-5-methyl-N-(3-(1-((1-methyl-1H-pyrazolo[3,4-b]pyrazin-6-yl)amino)ethyl)phenyl)nicotinamide (Compound 8)

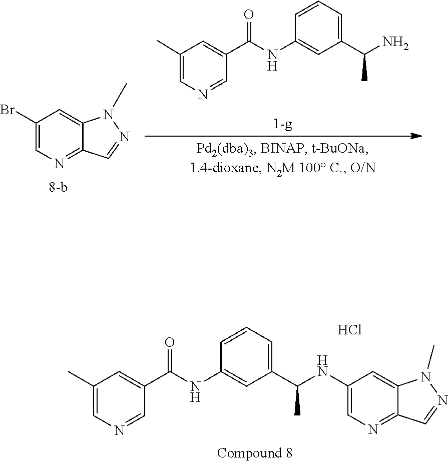 [SMILES: Cn1ncc2ncc(Br)cc21 ; Cc1cncc(C(=O)Nc2cccc([C@H](C)N)c2)c1 ; Cc1cncc(C(=O)Nc2cccc([C@H](C)Nc3cnc4cnn(C)c4c3)c2)c1 ; Cl]

To a stirred solution of 6-chloro-1-methyl-1H-pyrazolo[3,4-b]pyrazine (8-b) (50 mg, 0.29 mmol) in 1,4-dioxane (3 mL) was added (S)—N-(3-(1-aminoethyl)phenyl)-5-methylnicotinamide (1-g) (prepared as described in Example 1, Step 6) (90 mg, 0.35 mmol), Pd₂(dba)₃ (27 mg, 0.029 mmol), BINAP (18 mg, 0.029 mmol) and t-BuONa (85 mg, 0.89 mmol). The mixture was heated to reflux overnight under N₂ protection. The reaction mixture was partitioned between brine (50 mL) and EtOAc (50 mL). The organic layer was separated, dried (Na₂SO₄), filtered, and concentrated under reduced pressure. The crude residue was purified (Reverse-Phase Preparative HPLC; eluting with 0.1% HCl in H₂O/MeOH) to afford the hydrochloride salt of Compound 8 (9 mg, 8%) as a solid. 1H NMR (400 MHz, CD₃OD): δ 9.21 (s, 1H), 9.01 (s, 1H), 8.90 (s, 1H), 8.07 (s, 2H), 7.96 (s, 1H), 7.61-7.59 (m, 1H), 7.40-7.36 (m, 1H), 7.33-7.31 (m, 1H), 5.29-5.24 (q, 1H), 3.89 (s, 3H), 2.67 (s, 3H), 1.65-1.63 (d, 3H); LCMS Mass: 388.2 (M⁺+H).

### Example 9

### Synthesis of the hydrochloride salt of (S)—N-(3-(1-((2-ethyl-2H-pyrazolo[3,4-b]pyrazin-6-yl)amino)ethyl)phenyl)-5-methylnicotinamide (Compound 9)

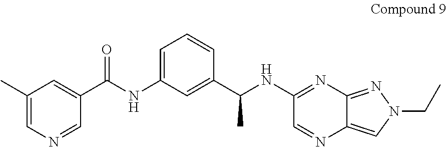 [SMILES: CCn1cc2ncc(N[C@@H](C)c3cccc(NC(=O)c4cncc(C)c4)c3)nc2n1]

### Step 1: Synthesis of 6-chloro-2-ethyl-2H-pyrazolo[3,4-b]pyrazine (9-b)

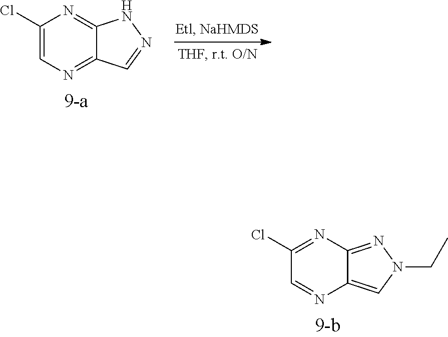

To a stirred solution of 6-chloro-1H-pyrazolo[3,4-b]pyrazine (9-a) (7.0 g, 45.3 mmol) in THF (100 mL) was added NaHMDS (1 M, 72.5 mL) followed by iodoethane (21.2 g, 136 mmol, 10.9 mL). The mixture was stirred at rt for 24 h. The reaction mixture was partitioned between water (100 mL) and EtOAc (100 mL). The organic layer was separated, dried (MgSO₄), filtered, and was concentrated under reduced pressure. The residue was triturated with ether/heptane (1:1) to give 4.4 g of compound 9-b. The filtrate was concentrated and purified (silica gel; eluting with 0-3% MeOH in DCM) to give an additional 1.1 g of material. The batches were combined to afford Compound 9-b (5.5 g, 67%) as a light brown solid. ¹H NMR (300 MHz, CDCl₃) δ ppm 8.50 (s, 1H) 8.26 (s, 1H) 4.56 (q, J=7.43 Hz, 2H) 1.71 (t, J=7.34 Hz, 3H); LCMS Mass: 182.9 (M⁺+H).

### Step 2: Synthesis of (S)-2-ethyl-N-(1-(3-nitrophenyl)ethyl)-2H-pyrazolo[3,4-b]pyrazin-6-amine (9-d)

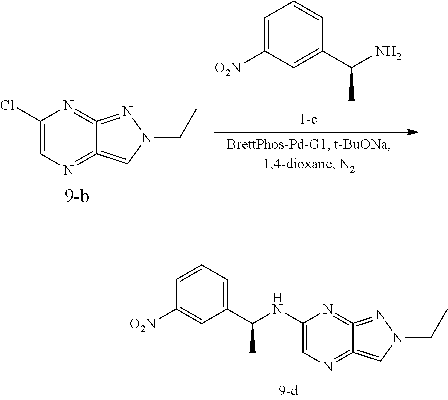

To a stirred solution of 6-chloro-2-ethyl-pyrazolo[3,4-b]pyrazine (9-b) (2.5 g, 13.7 mmol) in 1,4-dioxane (40 mL) at rt, was added (S)-1-(3-nitrophenyl)ethan-1-amine (1-c) prepared as described in Example 1, Step 2) (4.6 g, 27.4 mmol), t-BuONa (2.0 g, 20.5 mmol) and BrettPhos-Pd-G1 (1.09 g, 1.37 mmol). The mixture was heated to 90° C. under N₂ for 10 min. The reaction mixture was diluted with EtOAc (50 mL) and filtered through Celite. The filtrate was washed with brine (50 mL) and the aqueous layer was extracted with EtOAc (50 mL×2). The combined organic phase was washed with brine (50 mL), dried (MgSO₄), filtered, and concentrated under reduced pressure. The residue was purified (amine-functionalized silica; eluting with 0-100% EtOAc in heptane) to afford compound 9-d (1.7 g, 40%) as a brown solid. ¹H NMR (300 MHz, CDCl₃) δ ppm 8.27 (t, J=1.97 Hz, 1H) 8.04-8.14 (m, 1H) 7.94 (s, 1H) 7.93 (s, 1H) 7.80 (d, J=7.70 Hz, 1H) 7.49 (t, J=7.93 Hz, 1H) 5.46 (quin, J=6.85 Hz, 1H) 5.22 (br d, J=6.60 Hz, 1H) 4.33 (q, J=7.34 Hz, 2H) 1.65 (d, J=6.88 Hz, 3H) 1.56-1.61 (m, 3H); LCMS Mass: 313.89 (M⁺+H).

### Step 3: Synthesis of (S)—N-(1-(3-aminophenyl)ethyl)-2-ethyl-2H-pyrazolo[3,4-b]pyrazin-6-amine (9-e)

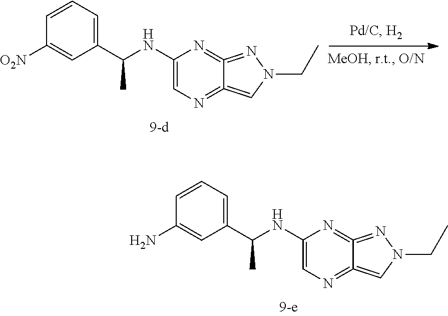

To a stirred solution of (S)-2-ethyl-N-(1-(3-nitrophenyl)ethyl)-2H-pyrazolo[3,4-b]pyrazin-6-amine (9-d) (1.7 g, 5.44 mmol) in methanol (60 mL) was added Pd/C (680 mg). The mixture was stirred at rt for 16 h under H₂ (1 atmosphere pressure). The reaction mixture was filtered through Celite and washed with methanol (30 mL). The filtrate was concentrated to dryness under reduced pressure to afford compound 9-e (1.53 g, 99%) as a light brown solid. ¹H NMR (300 MHz, CDCl₃) δ ppm 7.91 (s, 1H) 7.81 (s, 1H) 7.10-7.17 (m, 1H) 6.82 (d, J=7.70 Hz, 1H) 6.76 (t, J=1.97 Hz, 1H) 6.59 (ddd, J=7.93, 2.29, 0.87 Hz, 1H) 5.29 (quin, J=6.85 Hz, 1H) 5.04 (br d, J=7.43 Hz, 1H) 4.34 (q, J=7.34 Hz, 2H) 3.68 (br s, 2H) 1.58-1.63 (m, 6H); LCMS Mass: 283.65 (M⁺+H).

### Step 4: Synthesis of the hydrochloride salt of (S)—N-(3-(1-((2-ethyl-2H-pyrazolo[3,4-b] pyrazin-6-yl)amino)ethyl)phenyl)-5-methylnicotinamide (Compound 9)

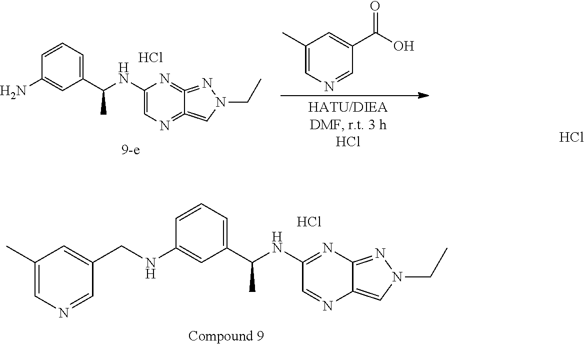

To a stirred solution of 5-methylnicotinic acid (33 mg, 0.24 mmol) in DMF (3 mL), was added (S)—N-(1-(3-aminophenyl)ethyl)-2-ethyl-2H-pyrazolo[3,4-b]pyrazin-6-amine (9-e) (56 mg, 0.20 mmol) HATU (95 mg, 0.25 mmol) and DIPEA (62 mg, 0.48 mmol). The mixture stirred at r.t for 3 h. The reaction mixture was partitioned between water (30 mL) and EtOAc (50 mL). The organic layer was separated and washed by brine (10 mL×2). The organic layer was separated, dried (Na₂SO₄), filtered, and was concentrated under reduced pressure. The crude residue was purified (Prep-TLC; eluting with MeOH:DCM=1:15) to afford the free base. The free base was stirred with 4M HCl in 1,4-dioxane and concentrated to afford the hydrochloride salt of Compound 9 (50 mg, 57%) as an off-white solid. ¹H NMR (400 MHz, DMSO-d₆) δ 10.82 (s, 1H), 9.19 (s, 1H), 8.88 (s, 1H), 8.72 (s, 1H), 8.38 (s, 2H), 8.11 (d, J=1.5 Hz, 1H), 7.81 (s, 1H), 7.69 (d, J=8.0 Hz, 1H), 7.35 (t, J=7.8 Hz, 1H), 7.22 (d, J=7.7 Hz, 1H), 5.10 (s, 1H), 4.22 (q, J=7.2 Hz, 2H), 2.51 (s, 3H), 1.51 (d, J=6.8 Hz, 3H), 1.40 (dd, J=7.9, 6.4 Hz, 3H); LCMS Mass: 402.1 (M⁺+H).

### Example 10

### Synthesis of the hydrochloride salt of (S)—N-(3-(1-((2-ethyl-2H-pyrazolo[3,4-b]pyrazin-6-yl)amino)ethyl)-4-fluorophenyl)-6-(trifluoromethyl)nicotinamide (Compound 10)

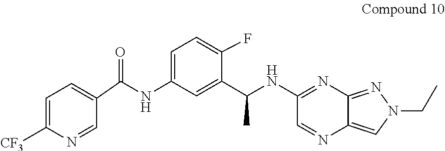 [SMILES: CCn1cc2ncc(N[C@@H](C)c3cc(NC(=O)c4ccc(C(F)(F)F)nc4)ccc3F)nc2n1]

### Step 1: Synthesis of (S,E)-N-(1-(2-fluoro-5-nitrophenyl)ethylidene)-2-methylpropane-2-sulfinamide (10-b)

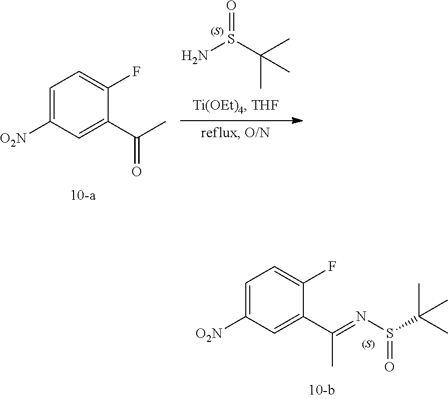

To a mixture of 1-(2-fluoro-5-nitrophenyl)ethan-1-one (10-a) (1.00 g, 5.46 mmol) in THF (10 mL) was added (S)-2-methylpropane-2-sulfinamide (0.99 g, 8.19 mmol) and Ti(OEt)₄ (2.45 g, 10.92 mmol). The mixture was stirred at 75° C. overnight. The mixture was cooled to room temperature and quenched with ice water (30 mL). EtOAc (100 mL) was added and the mixture was stirred at room temperature for 15 min. The mixture was filtered. The filtercake was washed with EtOAc (3×20 mL). The combined organic layers were washed with brine (30 mL) and dried over Na₂SO₄. The mixture was filtered and concentrated to dryness. The crude product was purified (silica gel; eluting with EtOAc:PE=1:8 to 1:4) to afford to compound 10-b (1.0 g, 64%) as a yellow solid. ¹H NMR (400 MHz, CDCl₃) δ 8.56 (dd, J=6.4, 2.9 Hz, 1H), 8.32 (dt, J=9.1, 3.6 Hz, 1H), 7.29 (t, J=9.4 Hz, 1H), 2.81 (d, J=3.5 Hz, 3H), 1.34 (s, 9H); LCMS

Mass: 287.0 (M⁺+H).

### Step 2: Synthesis of (S)—N—((S)-1-(2-fluoro-5-nitrophenyl)ethyl)-2-methylpropane-2-sulfinamide (10-c)

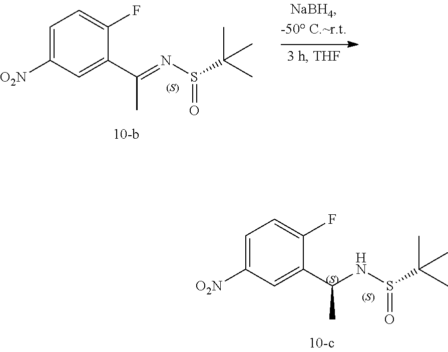

To a stirred solution of 10-b (800 mg, 2.79 mmol) in THF (10 mL) at −50° C., was added NaBH₄ (316 mg, 8.37 mmol). The mixture was stirred between 0° C. to r. t. for 3 h. The mixture was carefully quenched with sat. NH₄Cl solution (20 mL). The mixture was extracted with EtOAc (3×30 mL). The organic layer was washed with brine (15 mL) and dried over Na₂SO₄. The mixture was filtered, concentrated to dryness and purified (silica gel; eluting with EtOAc:PE=1:4 to 1:1) to afford compound 10-c (478 mg, 59%) as yellow solid. ¹H NMR (400 MHz, CDCl₃) δ 9.16 (s, 1H), 8.32 (dd, J=6.2, 2.8 Hz, 1H), 8.19 (ddd, J=8.9, 4.2, 2.7 Hz, 1H), 7.21 (t, J=9.1 Hz, 1H), 4.84 (t, J=6.4 Hz, 1H), 1.59 (d, J=6.7 Hz, 3H), 1.24 (s, 9H); LCMS Mass: 289.0 (MH⁺).

### Step 3: Synthesis of (S)—N—((S)-1-(5-amino-2-fluorophenyl)ethyl)-2-methylpropane-2-sulfinamide (10-d)

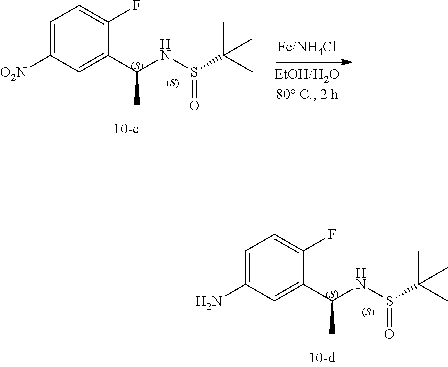

To a stirred solution of 10-c (100 mg, 0.35 mmol) in a mixture of EtOH (2 mL) and water (1 mL), was added Fe (58 mg, 1.04 mmol) and NH₄Cl (56 mg, 1.04 mmol). The mixture was stirred at 80° C. for 2 h. The mixture was filtered and concentrated to dryness. The crude product was diluted with a mixture of DCM (20 mL) and water (10 mL). The aqueous phase was extracted with DCM (2×10 mL). The combined organic layers were washed with brine (15 mL) and was dried over Na₂SO₄. The mixture was purified (Prep-TLC; eluting with MeOH:DCM=1:20) to afford compound 10-d (67 mg, 75%) as yellow solid. ¹H NMR (400 MHz, DMSO-d₆) δ 6.77 (dd, J=10.3, 8.7 Hz, 1H), 6.65 (dd, J=6.4, 2.8 Hz, 1H), 6.42 (ddd, J=8.7, 4.3, 2.9 Hz, 1H), 5.53 (d, J=6.6 Hz, 1H), 4.90 (s, 2H), 4.53 (m, 1H), 1.35 (d, J=6.8 Hz, 3H), 1.09 (s, 9H); LCMS Mass: 259.1 (MH⁺).

### Step 4: Synthesis of tert-butyl (3-((S)-1-(((S)-tert-butylsulfinyl)amino)ethyl)-4-fluorophenyl)carbamate (10-e)

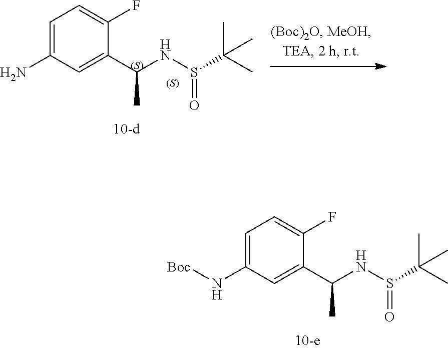

To a stirred solution of 10-d (6.4 g, 0.025 mol) in MeOH (150 mL), was added TEA (7.6 g, 0.075 mol) and (Boc)₂O (10.9 g, 0.050 mol). The mixture was stirred at r.t. for 2 h. The mixture was evaporated under reduced pressure. The reaction mixture was partitioned between water (150 mL) and EtOAc (150 mL). The organic layer was separated, dried (Na₂SO₄), filtered, and concentrated under reduced pressure. The crude residue was purified (silica gel; eluting with 30 to 40% EtOAc in PE) to afford compound 10-e (8.3 g, 93%) as an off-white solid. LCMS Mass: 359.2 (M⁺+H).

### Step 5: Synthesis of tert-butyl (S)-(3-(1-aminoethyl)-4-fluorophenyl)carbamate (10-f)

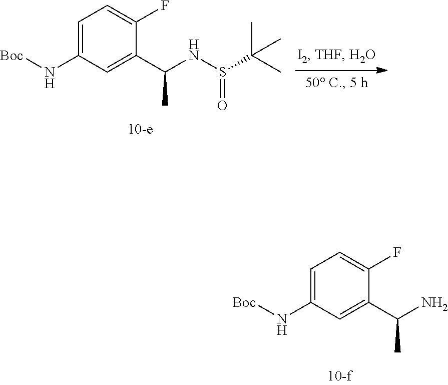 [SMILES: C[C@H](N[S@@](=O)C(C)(C)C)c1cc(NC(=O)OC(C)(C)C)ccc1F ; C[C@H](N)c1cc(NC(=O)OC(C)(C)C)ccc1F]

To a mixture of 10-e (8.3 g, 0.023 mol) in THF (80 mL) was added water (16 mL) and 12 (1.7 g, 0.007 mol). The mixture was stirred at 50° C. for 5 h. The mixture was cooled to r.t. and then diluted with saturated aqueous citric acid solution. The mixture was washed with EtOAc (200 mL×3). The aqueous phase was cooled to 0° C., then the pH was adjusted to 10 with NaOH (30% in water). The mixture was extracted with EtOAc (200 mL×3). The combined organic layers were dried (Na₂SO₄), filtered, and concentrated under reduced pressure to afford compound 10-f (5.5 g, 90%) as a yellow oil. LCMS Mass: 255.2 (M⁺+H).

### Step 6: Synthesis of tert-butyl(S)-(3-(1-((2-ethyl-2H-pyrazolo[3,4-b]pyrazin-6-yl)amino) ethyl)-4-fluorophenyl)carbamate (10-g)

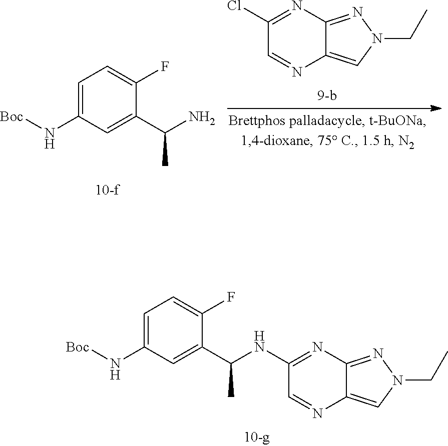 [SMILES: C[C@H](N)c1cc(NC(=O)OC(C)(C)C)ccc1F ; CCn1cc2ncc(Cl)nc2n1 ; CCn1cc2ncc(N[C@@H](C)c3cc(NC(=O)OC(C)(C)C)ccc3F)nc2n1]

To a stirred solution of 10-f (5.5 g, 0.022 mol) in 1,4-dioxane (100 mL) was added 6-chloro-2-ethyl-2H-pyrazolo[3,4-b]pyrazine (9-b) (prepared as described in Example 9, Step 1) (4.3 g, 0.024 mol), BrettPhos Palladacycle (0.8 g, 0.001 mol) and t-BuONa (6.2 g, 0.065 mol). The mixture was stirred at 75° C. for 1.5 h under a N₂ atmosphere. The reaction mixture was partitioned between brine (200 mL) and EtOAc (200 mL). The organic layer was separated, dried (Na₂SO₄), filtered, and concentrated under reduced pressure. The crude residue was purified (silica gel; eluting with EtOAc/PE=1/1) to afford compound 10-g (5.5 g, 63%) as a yellow solid. LCMS Mass: 401.3 (M⁺+H).

### Step 7: Synthesis of (S)—N-(1-(5-amino-2-fluorophenyl)ethyl)-2-ethyl-2H-pyrazolo[3,4-b] pyrazin-6-amine (10-h)

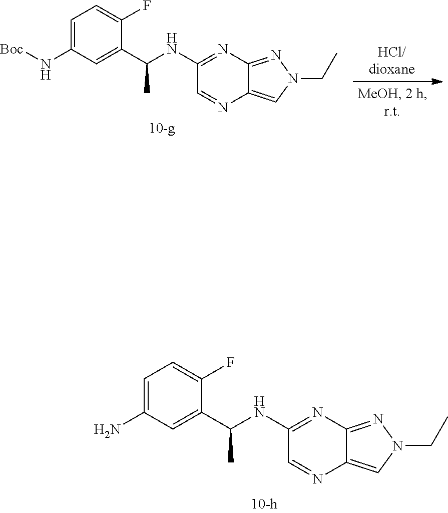 [SMILES: CCn1cc2ncc(N[C@@H](C)c3cc(NC(=O)OC(C)(C)C)ccc3F)nc2n1 ; CCn1cc2ncc(N[C@@H](C)c3cc(N)ccc3F)nc2n1]

To a stirred solution of 10-g (5.5 g, 13.5 mmol) in MeOH (40 mL), was added 4 M HCl in 1,4-dioxane (40 mL) and the mixture was stirred at r.t for 2 h. The mixture was evaporated under reduced pressure. The mixture was diluted with 3M HCl (200 mL) and washed with EtOAc (200 mL×3). The aqueous phase was cooled to 0° C., then the pH was adjusted to 12 with NaOH (30% in water). The mixture was extracted with EtOAc (200 mL×3). The combined organic layers were dried (Na₂SO₄), filtered, and concentrated under reduced pressure to afford compound 10-h (4.0 g, 96%) as yellow solid. ¹H NMR (400 MHz, DMSO-d₆) δ 8.32 (s, 1H), 8.04 (s, 1H), 7.87 (d, J=7.1 Hz, 1H), 6.80 (dd, J=10.4, 8.6 Hz, 1H), 6.54 (dd, J=6.5, 2.8 Hz, 1H), 6.37 (ddd, J=8.6, 4.2, 2.9 Hz, 1H), 5.18 (p, J=6.8 Hz, 1H), 4.87 (s, 2H), 4.21 (q, J=7.2 Hz, 2H), 1.44-1.38 (m, 6H); LCMS Mass: 301.2 (M⁺+H).

### Step 8: Synthesis of the hydrochloride salt of (S)—N-(3-(1-((2-ethyl-2H-pyrazolo[3,4-b] pyrazin-6-yl)amino)ethyl)-4-fluorophenyl)-6-(trifluoromethyl)nicotinamide (Compound 10)

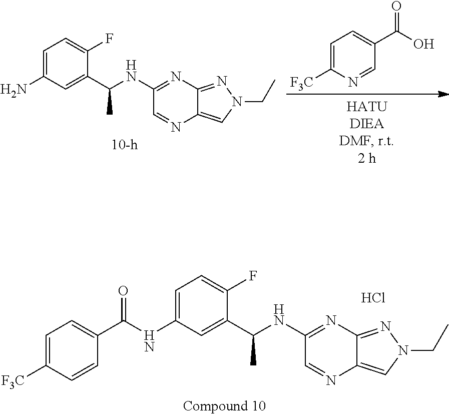

To a stirred solution of 10-h (50 mg, 0.167 mmol) and 6-(trifluoromethyl) nicotinic acid (32 mg, 0.83 mmol) in DMF (4 mL), was added HATU (95 mg, 0.25 mmol) and DIEA (64 mg, 0.5 mmol). The mixture was stirred at r.t. for 2.5 h. The reaction mixture was diluted with water, and extracted with EtOAc. The organic phase was washed with water, then brine, dried over Na₂SO₄, and concentrated under reduced pressure. The obtained residue was purified (Preparative HPLC; eluting with 0.1% HCl in H2O/MeOH) to afford the hydrochloride salt of Compound 10 (28 mg, 33%). ¹H NMR (400 MHz, DMSO-d₆) δ 10.65 (s, 1H), 9.19 (d, J=2.1 Hz, 1H), 8.51 (dd, J=8.2, 2.1 Hz, 1H), 8.33 (s, 1H), 8.13 (d, J=6.7 Hz, 1H), 8.10 (s, 1H), 8.07 (dd, J=8.2, 0.9 Hz, 1H), 7.81 (dd, J=6.9, 2.7 Hz, 1H), 7.67 (dd, J=8.8, 4.5, 2.7 Hz, 1H), 7.22 (dd, J=10.1, 8.8 Hz, 1H), 5.29 (m, 1H), 4.21 (q, J=7.2 Hz, 2H), 1.51 (d, J=6.9 Hz, 3H), 1.40 (t, J=7.3 Hz, 3H). LCMS Mass: 474.2 (M⁺+H).

### Example 11

### Synthesis of the hydrochloride salt of (S)—N-(3-(1-((2-ethyl-2H-pyrazolo[4,3-b]pyridin-6-yl)amino)ethyl)phenyl)-5-methylnicotinamide (Compound 11)

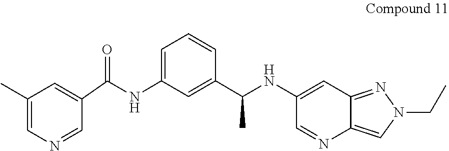 [SMILES: CCn1cc2ncc(N[C@@H](C)c3cccc(NC(=O)c4cncc(C)c4)c3)cc2n1]

### Step 1: Synthesis of 6-bromo-2-ethyl-2H-pyrazolo[4,3-b]pyridine (11-b)

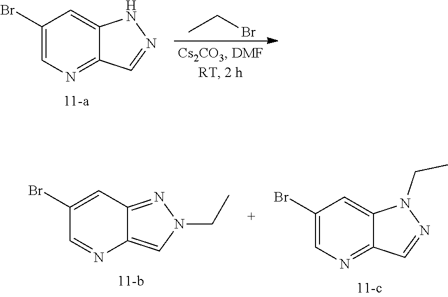

To a stirred solution of 6-bromo-1H-pyrazolo[4,3-b]pyridine (11-a) (1.00 g, 5.08 mmol) in DMF, was added cesium carbonate (3.3 g, 10.16 mmol) and bromoethane (1.1 g, 10.16 mmol) at r.t. The mixture was stirred at r.t. for 2 h. The mixture was concentrated and purified (silica gel; eluting with EtOAc/PE=1/1) to afford compound 11-b (420 mg, 37%) as a yellow solid and compound 11-c (640 mg, 56%) as a brown oil. Compound 11-b: ¹H NMR (400 MHz, DMSO-d₆) δ 8.77 (d, J=0.9 Hz, 1H), 8.53 (d, J=2.0 Hz, 1H), 8.43 (dd, J=2.1, 1.0 Hz, 1H), 4.49 (q, J=7.3 Hz, 2H), 1.52 (t, J=7.3 Hz, 3H); LCMS Mass: 226.1 (M⁺+H); Compound 11-c: ¹H NMR (400 MHz, CDCl₃) δ 8.47 (t, J=5.4 Hz, 1H), 8.09 (d, J=0.8 Hz, 1H), 7.83 (dd, J=1.8, 1.0 Hz, 1H), 4.28 (q, J=7.3 Hz, 2H), 1.42 (t, J=7.0 Hz, 3H); LCMS Mass: 226.1 (M⁺+H).

### Step 2: Synthesis of the hydrochloride salt of (S)—N-(3-(1-((2-ethyl-2H-pyrazolo[4,3-b] pyridin-6-yl)amino)ethyl)phenyl)-5-methylnicotinamide (Compound 11)

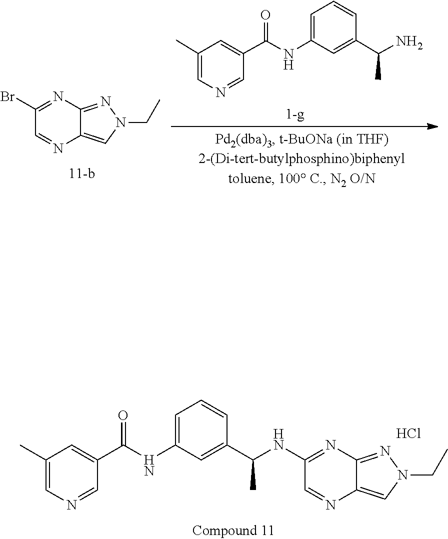 [SMILES: Cc1cncc(C(=O)Nc2cccc([C@H](C)N)c2)c1 ; CCn1cc2ncc(Br)nc2n1 ; CCn1cc2ncc(N[C@@H](C)c3cccc(NC(=O)c4cncc(C)c4)c3)nc2n1 ; Cl]

To a stirred solution of 6-bromo-2-ethyl-2H-pyrazolo[4,3-b]pyridine (11-b) (80 mg, 0.35 mmol) and (S)—N-(3-(1-aminoethyl)phenyl)-5-methylnicotinamide (1-g) (prepared as described in Example 1, Step 6) (100 mg, 0.4 mmol) in toluene, was added Pd₂(dba)₃ (32.05 mg, 0.035 mmol), t-BuONa (84 mg, 0.875 mmol) and 2-(di-tert-butylphosphino)biphenyl (10.5 mg, 0.035 mmol) and the mixture was stirred at 100° C. under N₂ atmosphere overnight. The mixture was extracted with DCM (20 mL×3). The organic phase was combined and dried (Na₂SO₄), filtered, and was concentrated under reduced pressure. The crude was purified (Reverse-Phase Preparative HPLC; eluting with 0.1% HCl in H₂O/MeOH) to afford afford the hydrochloride salt of Compound 11 (20 mg, 14%) as a solid. ¹H NMR (400 MHz, CD₃OD) δ 9.19 (s, 1H), 8.99 (s, 1H), 8.90 (d, J=20.9 Hz, 1H), 8.62 (s, 1H), 8.56 (s, 1H), 7.91 (s, 1H), 7.65 (s, 1H), 7.36 (s, 1H), 7.31 (t, J=7.1 Hz, 1H), 7.26 (d, J=20.9 Hz, 1H), 4.67 (dd, J=13.4, 6.7 Hz, 1H), 4.53-4.46 (m, 2H), 2.64 (s, 3H), 1.62 (s, 3H), 1.56 (d, J=6.7 Hz, 3H); LCMS Mass: 401.2 (M⁺+H).

### Example 12

### Synthesis of the hydrochloride salt of (5)-5-methyl-N-(3-(1-((3-methyl-2-oxo-2,3-dihydro-1H-imidazo[4,5-b]pyrazin-5-yl)amino)ethyl)phenyl)nicotinamide (Compound 12)

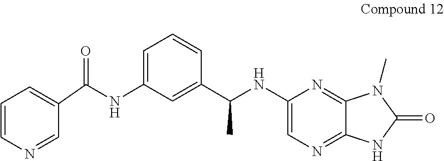 [SMILES: C[C@H](Nc1cnc2[nH]c(=O)n(C)c2n1)c1cccc(NC(=O)c2cccnc2)c1]

### Step 1: Synthesis of 6-bromo-N2-methylpyrazine-2,3-diamine (12-b)

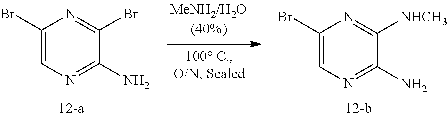 [SMILES: Nc1ncc(Br)nc1Br ; CNc1nc(Br)cnc1N]

A solution of 3,5-dibromopyrazin-2-amine (12-a) (3 g, 11.96 mmol) in MeNH₂/THF (2 M) (30 mL) was stirred in a sealed tube at 100° C. overnight. The mixture was concentrated and was purified (silica gel; eluting with ethyl acetate:Petroleum ether=1:2) to afford compound 12-b (1.96 g, 81%) as a white solid. ¹H NMR (400 MHz, Chloroform-d) δ 7.46 (s, 1H), 4.49 (s, 1H), 3.03 (d, J=3.9 Hz, 3H). LCMS Mass: 203 (M⁺+H).

### Step 2: Synthesis of 6-bromo-1-methyl-1,3-dihydro-2H-imidazo[4,5-b]pyrazin-2-one (12-c)

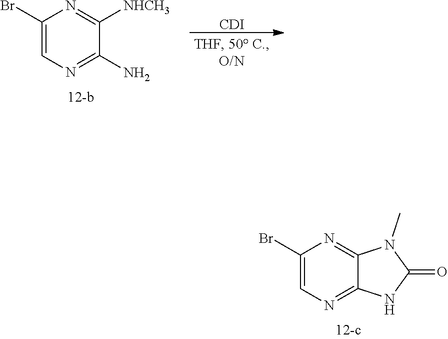

A solution of 12-b (737 mg, 3.65 mmol) and CDI (1.479 g, 9.12 mmol) in THF was stirred at 50° C. overnight. The mixture was cooled to r.t. and ethyl acetate (30 mL) and water (20 mL) were added. The organic layer was separated and concentrated, and the crude product was purified (Silica gel; eluting with EA:PE=1:4) to afford compound 12-c (800 mg, 96%) as a white solid. ¹H NMR (400 MHz, Chloroform-d) δ 9.33 (s, 1H), 8.05 (s, 1H), 3.49 (s, 3H); LCMS Mass: 229 (M⁺+H).

### Step 3: Synthesis of 5-bromo-1-(4-methoxybenzyl)-3-methyl-1,3-dihydro-2H-imidazo[4,5-b]pyrazin-2-one (12-d)

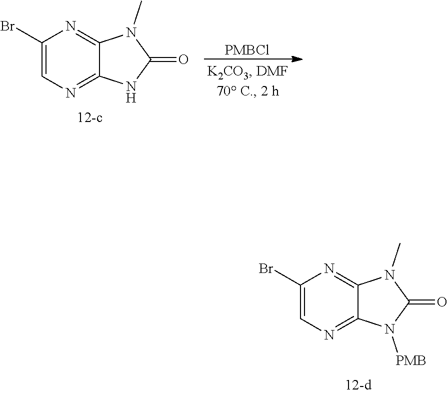

To a solution of 12-c (500 mg, 2.2 mmol), PMBCl (412 g, 2.64 mmol) and K₂CO₃ (456 mg, 3.3 mmol) in DMF was stirred at 70° C. for 2 h. The mixture was cooled down to r.t. and DCM (20 mL) and water (20 mL) were added. The organic layer was separated and concentrated to afford compound 12-d (500 mg, 65%) as a white solid. LCMS Mass: 348.9 (M⁺+H).

### Step 4: Synthesis of (S)-1-(4-methoxybenzyl)-3-methyl-5-((1-(3-nitrophenyl)ethyl) amino)-1,3-dihydro-2H-imidazo[4,5-b]pyrazin-2-one (12-e)

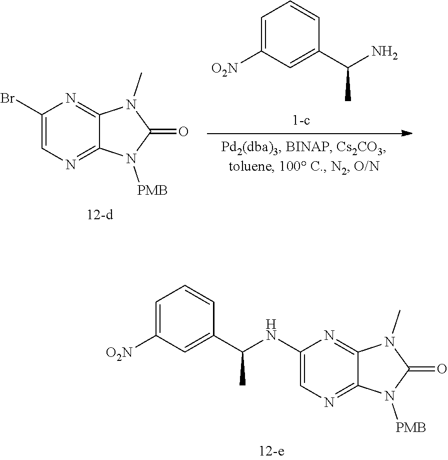

To a stirred solution of 12-d (200 mg, 0.575 mmol) in toluene (8 mL) was added Cs₂CO₃ (561.75 mg, 1.725 mmol), (S)-1-(3-nitrophenyl)ethan-1-amine (1-c) (prepared as described in Example 1, Step 2) (105 mg, 0.63 mmol), BINAP (35.78 mg, 0.057 mmol) and Pd₂(dba)₃ (52.62 mg, 0.057 mmol). The mixture was heated to 100° C. overnight under a N₂ atmosphere. The reaction mixture was concentrated and the residue was dissolved in EtOAc (20 mL). The mixture was washed by water (40 mL) and then brine (40 mL). The organic layer was separated, dried (Na₂SO₄), filtered, and concentrated under reduced pressure. The crude residue was purified (silica gel; eluting with 0˜50% EtOAc in PE) to afford compound 12-e (100 mg, 40%) as an orange solid. LCMS Mass: 435.20 (M⁺+H).

### Step 5: Synthesis of (5)-5-((1-(3-aminophenyl)ethyl)amino)-1-(4-methoxybenzyl)-3-methyl-1,3-dihydro-2H-imidazo[4,5-b]pyrazin-2-one (12-f)

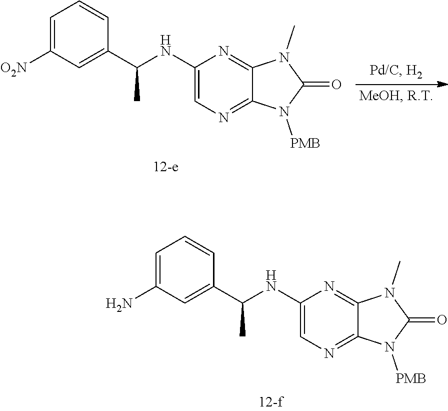

To a stirred solution of 12-e (100 mg, 0.230 mmol) in methanol (10 mL), was added Pd/C (10 mg) and the mixture was stirred at r.t for 4 h under a H2 atmosphere. The reaction mixture was filtered through celite and the filter cake was washed with methanol (15 mL). The filtrate was concentrated to dryness under reduced pressure to afford compound 12-f (90 mg, 97%) as a yellow solid. LCMS Mass: 405.25 (M⁺+H).

### Step 6: Synthesis of (S)—N-(3-(1-((1-(4-methoxybenzyl)-3-methyl-2-oxo-2,3-dihydro-1H-imidazo[4,5-b]pyrazin-5-yl)amino)ethyl)phenyl)-5-methylnicotinamide (12-g)

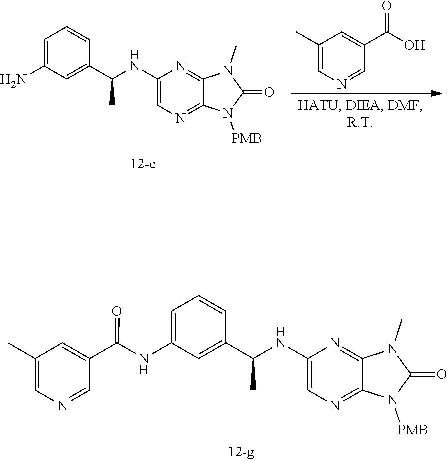

To a stirred solution of 12-f (90 mg, 0.222 mmol) in DMF (2 mL), was added HATU (169.2 mg, 0.444 mmol), DIPEA (86.26 mg, 0.668 mmol) and 5-methylnicotinic acid (45.77 mg, 0.333 mmol) and the mixture stirred at r.t for 2 h. The reaction mixture was partitioned between water (40 mL) and EtOAc (20 mL). The organic layer was separated and washed with brine (25 mL×2). The organic layer was separated, dried (Na₂SO₄), filtered, and was concentrated under reduced pressure. The crude residue was purified (Reverse-Phase C18 column; eluting with 60% MeOH in water) to afford compound 12-g (100 mg, 86%) as yellow solid. LCMS Mass: 524.30 (M⁺+H).

### Step 7: Synthesis of the hydrochloride salt of (S)-5-methyl-N-(3-(1-((3-methyl-2-oxo-2,3-dihydro-1H-imidazo[4,5-b]pyrazin-5-yl)amino)ethyl)phenyl)nicotinamide (Compound 12)

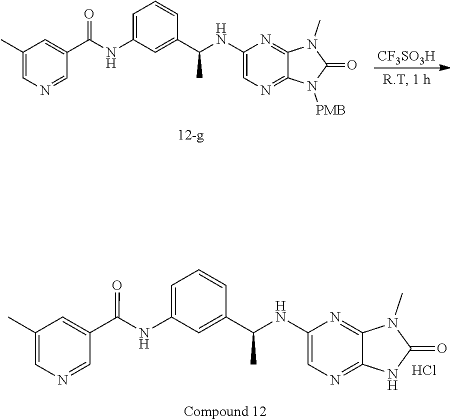

A solution of 12-g (60 mg, 0.11 mmol) in trifluoromethanesulfonic acid (1 mL) was stirred at r.t for 1 h. The reaction mixture was concentrated and purified (Preparative Reverse-Phase HPLC; eluting with 0.1% HCl in H₂O/MeOH) to afford the hydrochloride salt of Compound 12 (1.5 mg, 3%) as a solid. ¹H NMR (400 MHz, CD₃OD): δ 9.17 (s, 1H), 8.96 (s, 1H), 8.88 (s, 1H), 7.84 (s, 1H), 7.56 (d, 1H), 7.36 (t, 1H), 7.62 (d, 1H), 7.22 (s, 1H), 5.34 (t, 1H), 3.32 (s, 3H), 2.66 (s, 3H), 1.59 (d, 3H). LCMS Mass: 404.25 (M⁺+H).

### Example 13

### Synthesis of the hydrochloride salt of (S)-5-methyl-N-(3-(1-((3-methyl-1H-pyrrolo[2,3-b] pyridin-5-yl)amino)ethyl)phenyl)nicotinamide (Compound 13)

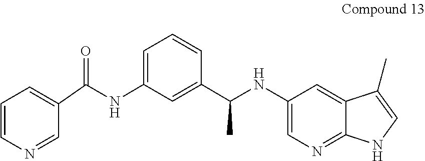 [SMILES: Cc1c[nH]c2ncc(N[C@@H](C)c3cccc(NC(=O)c4cccnc4)c3)cc12]

### Step 1: Synthesis of 5-bromo-3-methyl-1-((2-(trimethylsilyl)ethoxy)methyl)-1H-pyrazolo [3,4-b]pyridine (13-b)

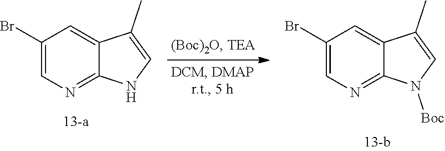 [SMILES: Cc1c[nH]c2ncc(Br)cc12 ; Cc1cn(C(=O)OC(C)(C)C)c2ncc(Br)cc12]

To a stirred solution of 5-bromo-3-methyl-1H-pyrrolo[2,3-b]pyridine (13-a) (300 mg, 1.421 mmol) in DCM (5 mL) was added (Boc)₂O (620 mg, 2.843 mmol), TEA (431 mg, 4.264 mmoL) and DMAP (17 mg, 0.142 mmoL). The mixture was stirred at 20° C. for 5 h. The mixture was concentrated and purified (silica gel; eluting with EtOAc/PE=1/50) to afford compound 13-b (350 mg, 79%) as an off-white solid. ¹H NMR (400 MHz, CD₃OD) δ 8.38 (d, J=2.2 Hz, 1H), 8.16 (d, J=2.2 Hz, 1H), 7.53 (d, J=1.2 Hz, 1H), 2.25 (s, 3H), 1.66 (s, 9H); LCMS Mass: 311.05 (M⁺+H).

### Step 2: Synthesis of the hydrochloride salt of (S)-5-methyl-N-(3-(1-((3-methyl-1H-pyrrolo[2,3-b]pyridin-5-yl)amino)ethyl)phenyl)nicotinamide (Compound 13)

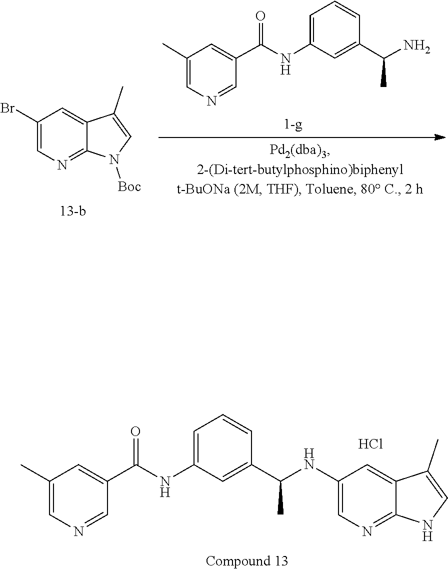 [SMILES: Cc1cn(C(=O)OC(C)(C)C)c2ncc(Br)cc12 ; Cc1cncc(C(=O)Nc2cccc([C@H](C)N)c2)c1 ; Cc1cncc(C(=O)Nc2cccc([C@H](C)Nc3cnc4[nH]cc(C)c4c3)c2)c1 ; Cl]

To a mixture of 13-b (350 mg, 1.125 mmol) in 1, 4-dioxane (5 mL), was added Pd₂(dba)₃ (103 mg, 0.113 mmol), (S)—N-(3-(1-aminoethyl)phenyl)-5-methylnicotinamide (1-g) (prepared as described in Example 1, Step 6) (287 mg, 1.125 mmol), 2-(di-tert-butylphosphino)biphenyl (36 mg, 0.113 mmol) and t-BuONa (2M, THF) (1.7 ml, 3.375 mmol). The mixture was stirred at 80° C. for 2 h under a nitrogen atmosphere and was cooled down to room-temperature. The mixture was concentrated, and the residue was purified (reverse-phase column; eluting with MeOH/water/HCl) to afford the hydrochloride salt of Compound 13 (11 mg) as a yellow solid. ¹H NMR (400 MHz, CD₃OD) δ 9.21 (s, 1H), 9.01 (s, 1H), 8.90 (s, 1H), 7.94 (d, J=2.0 Hz, 2H), 7.73 (s, 1H), 7.65 (d, J=8.1 Hz, 1H), 7.40 (t, J=7.9 Hz, 1H), 7.33-7.25 (m, 2H), 4.72 (q, J=6.7 Hz, 1H), 2.67 (s, 3H), 2.27 (s, 3H), 1.70 (d, J=6.7 Hz, 3H); LCMS Mass: 386.30 (M⁺+H).

### Example 14

### Synthesis of the hydrochloride salt of (5)-5-methyl-N-(3-(1-((3-methyl-1H-pyrazolo[3,4-b]pyridin-5-yl)amino)ethyl)phenyl)nicotinamide (Compound 14)

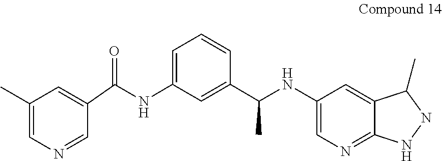 [SMILES: Cc1cncc(C(=O)Nc2cccc([C@H](C)Nc3cnc4c(c3)C(C)NN4)c2)c1]

### Step 1: Synthesis of 5-bromo-3-methyl-1-((2-(trimethylsilyl)ethoxy)methyl)-1H-pyrazolo [3,4-b]pyridine (14-b)

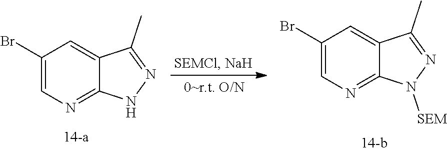 [SMILES: Cc1n[nH]c2ncc(Br)cc12 ; Cc1nn(COCC[Si](C)(C)C)c2ncc(Br)cc12]

To an ice cooled solution of 5-bromo-3-methyl-1H-pyrazolo[3,4-b] pyridine (14-a) (400 mg, 1.9 mmol) in THF (20 mL), was added NaH (112 mg, 2.8 mmol) and SEMCl (466.8 mg, 2.8 mmol) and the mixture was stirred between 0° C. and r.t. for 16 h. The reaction mixture was partitioned between water (20 mL) and EtOAc (100 mL). The organic layer was separated and washed with brine (10 mL×2). The organic layer was separated, dried (Na₂SO₄), filtered, and concentrated under reduced pressure. The crude residue was purified by C18 column chromatography to afford compound 14-b (270 mg, 41%) as an off-white solid. LCMS Mass: 342.0 and 344.5 (MH⁺).

### Step 2: Synthesis of (S)-5-methyl-N-(3-(1-((3-methyl-1-((2-(trimethylsilyl)ethoxy) methyl)-1H-pyrazolo[3,4-b]pyridin-5-yl)amino)ethyl)phenyl)nicotinamide (14-c)

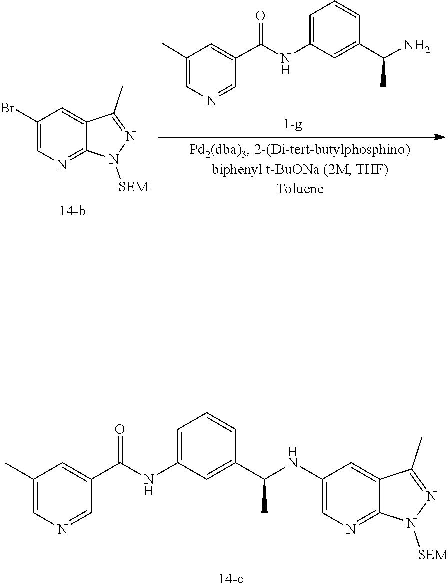 [SMILES: Cc1nn(COCC[Si](C)(C)C)c2ncc(Br)cc12 ; Cc1cncc(C(=O)Nc2cccc([C@H](C)N)c2)c1 ; Cc1cncc(C(=O)Nc2cccc([C@H](C)Nc3cnc4c(c3)c(C)nn4COCC[Si](C)(C)C)c2)c1]

To a stirred solution of 14-b (270 mg, 0.79 mmol) in toluene (10 mL) was added (S)—N-(3-(1-aminoethyl)phenyl)-5-methylnicotinamide (1-g) (prepared as described in Example 1, Step 6) (201 mg, 0.79 mmol), Pd₂(dba)₃ (72 mg, 0.079 mmol), 2-(di-tert-butylphosphino)biphenyl (47 mg, 0.16 mmol), and t-BuONa (0.8 mL, 1.6 mmol). The mixture was heated to reflux under N₂ atmosphere for 2 h. The reaction mixture was partitioned between brine (50 mL) and EtOAc (50 mL). The organic layer was separated, dried (Na₂SO₄), filtered, and concentrated under reduced pressure. The crude residue was purified by C18 column chromatography to afford compound 14-c (100 mg, 24%) as an off-white solid. LCMS Mass: 517.3 (M⁺+H).

### Step 3: Synthesis of the hydrochloride salt of (S)-5-methyl-N-(3-(1-((3-methyl-1H-pyrazolo[3,4-b]pyridin-5-yl)amino)ethyl)phenyl)nicotinamide (Compound 14)

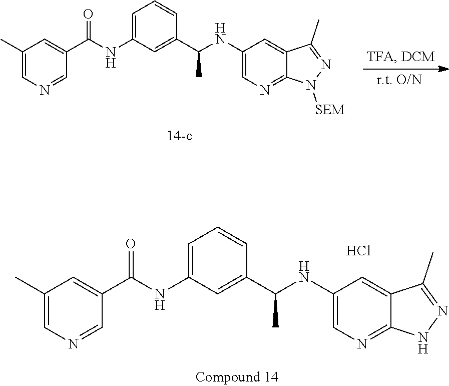 [SMILES: Cc1cncc(C(=O)Nc2cccc([C@H](C)Nc3cnc4c(c3)c(C)nn4COCC[Si](C)(C)C)c2)c1 ; Cc1cncc(C(=O)Nc2cccc([C@H](C)Nc3cnc4[nH]nc(C)c4c3)c2)c1 ; Cl]

To a stirred solution of 14-c (100 mg, 0.19 mmol) in DCM (10 mL) was added TFA (1 mL, 13.4 mmol). The mixture was stirred at r.t. O/N. The reaction mixture was concentrated under reduced pressure. The crude residue was purified (Preparative-HPLC; eluting with 0.1% HCl in H₂O/MeOH) to afford the hydrochloride salt of Compound 14 (18 mg, 34%) as a solid. ¹H NMR (400 MHz, CD₃OD) δ 9.24 (s, 1H), 9.04 (s, 1H), 8.93 (s, 1H), 8.38 (d, J=2.5 Hz, 1H), 8.09 (d, J=2.4 Hz, 1H), 7.97 (s, 1H), 7.72 (d, J=9.2 Hz, 1H), 7.43 (t, J=7.9 Hz, 1H), 7.29 (d, J=7.8 Hz, 1H), 4.85 (s, 1H), 2.69 (s, 3H), 2.58 (s, 3H), 1.81 (d, J=6.8 Hz, 3H); LCMS Mass: 387.2 (M⁺+H).

### Example 15

### Synthesis of the hydrochloride salt of (5)-5-methyl-N-(3-(1-((7-methyl-5H-pyrrolo[2,3-b] pyrazin-2-yl)amino)ethyl)phenyl)nicotinamide (Compound 15)

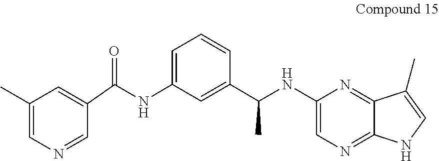 [SMILES: Cc1cncc(C(=O)Nc2cccc([C@H](C)Nc3cnc4[nH]cc(C)c4n3)c2)c1]

### Step 1: Synthesis of N-allyl-3,5-dibromopyrazin-2-amine (15-b)

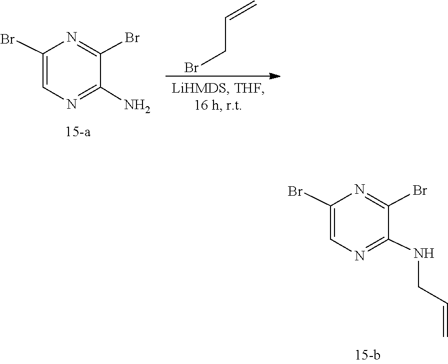

To a stirred solution of 3,5-dibromopyrazin-2-amine (15-a) (20 g, 0.079 mol) in THF at room temperature, was added LiHMDS (94.90 mL, 0.095 mol). After 2 h, 3-bromoprop-1-ene (19.10 g, 0.158 mol) was added. The mixture was stirred at r.t. for 16 h. The mixture was quenched with saturated NH₄Cl, and extracted with EtOAc. The organic layer was washed with brine. The mixture was concentrated and purified (Silica gel; eluting with EtOAc/PE=1/100) to afford compound 15-b (14 g, 60%) as a black oil. LCMS Mass: 293.8 (M⁺+H)

### Step 2: Synthesis of 2-bromo-7-methyl-5H-pyrrolo[2,3-b]pyrazine (15-c)

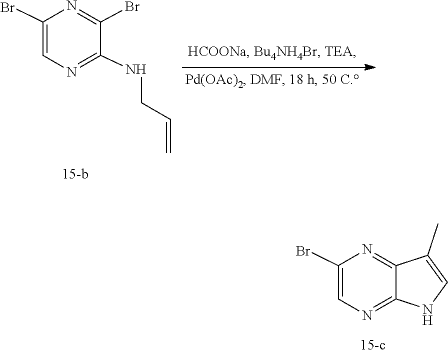

To a stirred solution of 15-b (14 g, 0.048 mol) in DMF (100 mL) was added HCOONa (0.8 g, 0.012 mol), Pd(OAc)₂ (1.1 g, 0.005 mol), Bu₄NH₄Br (2.3 g, 0.007 mol) and TEA (11.6 g, 0.115 mol). The mixture was stirred at 50° C. for 18 h under a N₂ atmosphere. The reaction mixture was filtered through Celite and the filter cake was washed by methanol. The filtrate was concentrated under reduced pressure. The crude residue was purified (Silica gel; eluting with EtOAc/PE=1/2) to afford compound 15-c (1.6 g, 16%) as a black solid. LCMS Mass: 213.9 (M⁺+H).

### Step 3: Synthesis of tert-butyl 2-bromo-7-methyl-5H-pyrrolo[2,3-b]pyrazine-5-carboxylate (15-d)

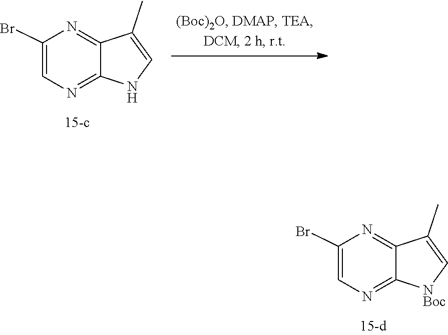 [SMILES: Cc1c[nH]c2ncc(Br)nc12 ; Cc1cn(C(=O)OC(C)(C)C)c2ncc(Br)nc12]

To a stirred solution of 15-c (1.6 g, 7.5 mmol) in DCM (30 mL), was added DMAP (0.4 g, 3.5 mmol), TEA (1.5 g, 15.1 mmol) and (Boc)₂O (3.3 g, 15.1 mmol) and the mixture was stirred at r.t. for 2 h. The mixture was evaporated under reduced pressure and the reaction mixture was partitioned between water (80 mL) and EtOAc (60 mL). The organic layer was separated, dried (Na₂SO₄), filtered, and concentrated under reduced pressure. The crude residue was purified (silica gel; eluting with 0 to 10% EtOAc in PE) to afford compound 15-d (1.76 g, 74%) as a white solid. ¹H NMR (400 MHz, CD₃OD) δ 8.43 (s, 1H), 7.90 (s, 1H), 2.32-2.28 (m, 3H), 1.67 (s, 9H); LCMS Mass: 256.0 (MH⁺−56).

### Step 4: Synthesis of tert-butyl (S)-7-methyl-2-((1-(3-(5-methylnicotinamido)phenyl) ethyl)amino)-5H-pyrrolo[2,3-b]pyrazine-5-carboxylate (15-e)

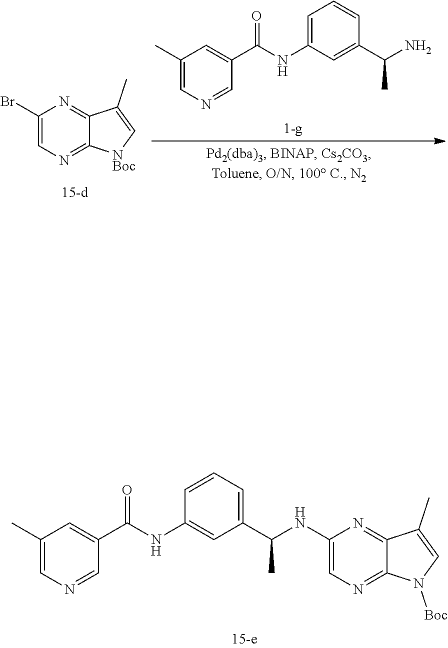

To a stirred solution of 15-d (150 mg, 0.48 mmol) in toluene (15 mL), was added (S)—N-(3-(1-amino ethyl)phenyl)-5-methylnicotinamide (1-g) (prepared as described in Example 1, Step 6) (122 mg, 0.48 mmol), Pd₂(dba)₃ (44 mg, 0.048 mmol), BINAP (30 mg, 0.048 mmol) and Cs₂CO₃ (470 mg, 1.44 mmol). The mixture was heated to 100° C. for overnight under a N₂ atmosphere. The reaction mixture was partitioned between brine (100 mL) and EtOAc (50 mL). The organic layer was separated, dried (Na₂SO₄), filtered, and concentrated under reduced pressure. The crude residue was purified (silica gel; eluting with 0 to 50% EtOAc in PE) to afford compound 15-e (93 mg, 40%) as yellow solid. LCMS Mass: 487.30 (M⁺+H).

### Step 5: Synthesis of the hydrochloride salt of (S)-5-methyl-N-(3-(1-((7-methyl-5H-pyrrolo[2,3-b]pyrazin-2-yl)amino)ethyl)phenyl)nicotinamide (Compound 15)

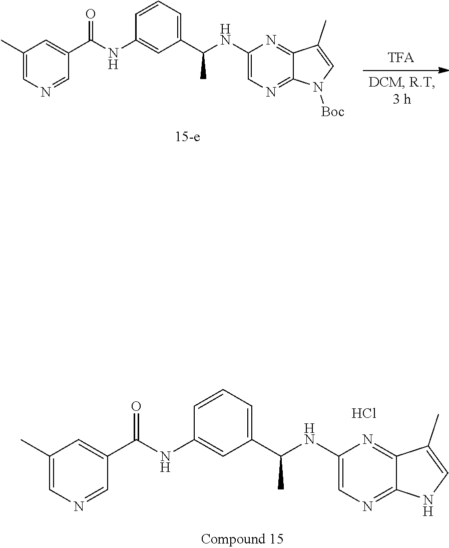 [SMILES: Cc1cncc(C(=O)Nc2cccc([C@H](C)Nc3cnc4c(n3)c(C)cn4C(=O)OC(C)(C)C)c2)c1 ; Cc1cncc(C(=O)Nc2cccc([C@H](C)Nc3cnc4[nH]cc(C)c4n3)c2)c1 ; Cl]

To a stirred solution of compound 15-e (93 mg, 0.19 mmol) in DCM (5 mL), was added TFA (1 mL) and the mixture was stirred at r.t for 3 h. The mixture was evaporated under reduced pressure and the crude residue was purified (Preparative Reverse-Phase HPLC; eluting with 0.1% HCl in H₂O/MeOH) to afford the hydrochloride salt of Compound 15 (32 mg, 44%) as a solid. ¹H NMR (400 MHz, DMSO-d₆): δ 11.88 (s, 1H), 10.78 (s, 1H), 9.14 (s, 1H), 8.82 (s, 1H), 8.61 (s, 1H), 7.88 (s, 2H), 7.70-7.67 (d, 1H), 7.47 (s, 1H), 7.38-7.34 (t, 1H), 7.29-7.27 (d, 1H), 5.29 (m, 1H), 2.48 (s, 3H), 2.20 (s, 3H), 1.56-1.54 (d, 3H); LCMS Mass: 387.20 (M⁺+H).

### Example 16

### Synthesis of the hydrochloride salt of (S)-5-methyl-N-(3-(1-((3-methyl-1H-pyrazolo[3,4-b]pyrazin-5-yl)amino)ethyl)phenyl)nicotinamide (Compound 16)

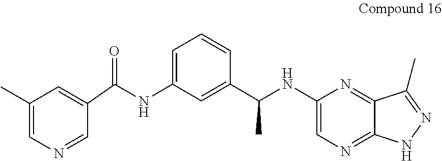 [SMILES: Cc1cncc(C(=O)Nc2cccc([C@H](C)Nc3cnc4[nH]nc(C)c4n3)c2)c1]

### Step 1: Synthesis of 3-methyl-1-trityl-1H-pyrazolo[3,4-b]pyrazine (16-b)

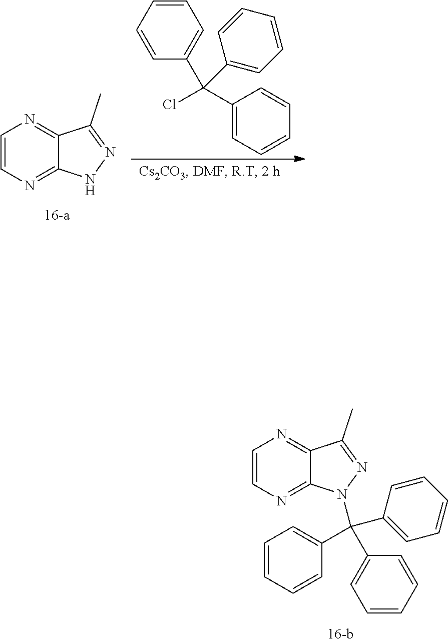

To a stirred solution of 3-methyl-1H-pyrazolo[3,4-b]pyrazine (16-a) (2.0 g, 14.9 mmol) in DMF (30 mL) was added trityl chloride (6.23 g, 22.4 mmol) and Cs₂CO₃ (9.72 g, 29.8 mmol). The mixture was stirred at r.t. for 2 h. The mixture was diluted with EtOAc (80 mL) and washed by water (200 mL), and brine (100 mL×2). The organic layer was separated, dried (Na₂SO₄), filtered, and concentrated under reduced pressure. The crude residue was purified (silica gel; eluting with 0˜10% EtOAc in PE) to afford compound 16-b (5.22 g, 93%) as an off white solid. ¹H NMR (400 MHz, DMSO-d₆): δ 8.49 (d, 1H), 8.18 (d, 1H), 7.29-7.20 (m, 15H), 2.52 (s, 3H).

### Step 2: Synthesis of 3-methyl-1-trityl-1H-pyrazolo[3,4-b]pyrazine 4-oxide (16-c)

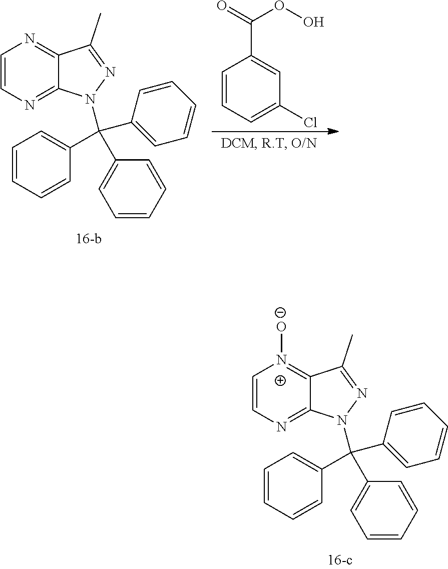

To a stirred solution of 16-b (5.20 g, 13.8 mmol) in DCM (50 mL) was added 3-chlorobenzoperoxoic acid (3.58 g, 20.7 mmol). The mixture was stirred at r.t. for 16 h. The mixture was treated with saturated Na₂SO₃ (80 mL) and extracted with DCM (50 mL). The organic layer was washed by brine (100 mL). The organic layer was separated, dried (Na₂SO₄), filtered, and concentrated under reduced pressure. The crude residue was purified (silica gel; eluting with 0˜20% EtOAc in PE) to afford compound 16-c (4.19 g, 77%) as a yellow solid. ¹H NMR (400 MHz, DMSO-d₆): δ 8.13-8.11 (m, 2H), 7.30-7.19 (m, 15H), 2.60 (s, 3H).

### Step 3: Synthesis of 5-bromo-3-methyl-1H-pyrazolo[3,4-b]pyrazine (16-d)

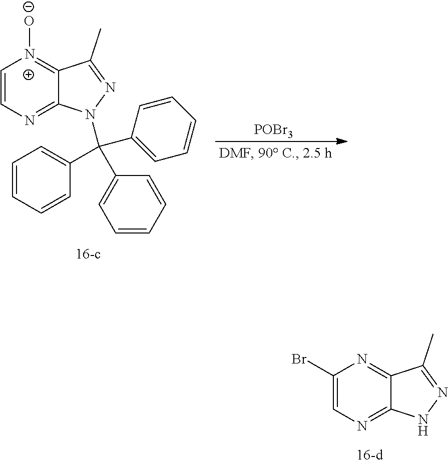

To a stirred solution of 16-c (4.19 g, 10.6 mmol) in DMF (50 mL) was added POBr₃ (3.67 g, 12.8 mmol) at 0° C. The mixture was stirred at 90° C. for 2.5 h. The mixture was cooled down to r.t and the pH was adjusted to 8 with saturated aq Na₂CO₃ solution. The mixture was extracted with EtOAc (80 ml) and washed by water (100 ml×2), then brine (100 ml). The organic layer was separated, dried (Na₂SO₄), filtered, and concentrated under reduced pressure. The crude residue was purified (silica gel; eluting with 0˜20% EtOAc in PE) to afford compound 16-d (4.19 g, 77%) as a white solid. ¹H NMR (400 MHz, DMSO-d₆): δ 13.92 (s, 1H), 8.68 (s, 1H), 2.52 (s, 3H); LCMS Mass: 212.95 (M⁺+H).

### Step 4: Synthesis of tert-butyl 5-bromo-3-methyl-1H-pyrazolo[3,4-b]pyrazine-1-carboxylate (16-e)

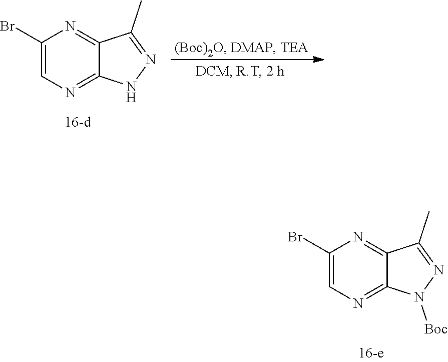

To a stirred solution of 16-d (1.23 g, 5.77 mmol) in DCM (30 mL), was added TEA (1.46 g, 14.4 mmol) and (Boc)₂O (1.89 g, 8.66 mmol) and the mixture was stirred at r.t. for 2 h. The mixture was evaporated under reduced pressure and the reaction mixture was partitioned between water (100 mL) and EtOAc (80 mL). The organic layer was separated, dried (Na₂SO₄), filtered, and concentrated under reduced pressure. The crude residue was purified (silica gel; eluting with 0˜20% EtOAc in PE) to afford compound 16-e (1.02 g, 57%) as an off-white solid. ¹H NMR (400 MHz, CDCl₃): δ 8.69 (s, 1H), 2.67 (s, 3H), 1.72 (s, 9H).

### Step 5: Synthesis of tert-butyl (S)-3-methyl-5-((1-(3-nitrophenyl)ethyl)amino)-1H-pyrazolo[3,4-b]pyrazine-1-carboxylate (16-f)

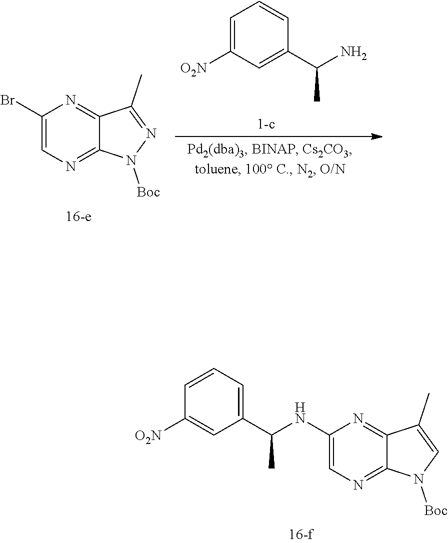 [SMILES: Cc1nn(C(=O)OC(C)(C)C)c2ncc(Br)nc12 ; C[C@H](N)c1cccc([N+](=O)[O-])c1 ; Cc1cn(C(=O)OC(C)(C)C)c2ncc(N[C@@H](C)c3cccc([N+](=O)[O-])c3)nc12]

To a stirred solution of 16-e (626 mg, 2.0 mmol) in toluene (30 mL) was added (S)-1-(3-nitrophenyl)ethan-1-amine (1-c) (prepared as described in Example 1, Step 2) (332 mg, 2.0 mmol), Pd₂(dba)₃ (184 mg, 0.2 mmol), BINAP (125 mg, 0.2 mmol) and Cs₂CO₃ (1.95 g, 6.0 mmol). The mixture was heated to reflux overnight under a N₂ atmosphere. The reaction mixture was partitioned between brine (50 mL) and EtOAc (50 mL). The organic layer was separated, dried (Na₂SO₄), filtered, and concentrated under reduced pressure. The crude residue was purified (silica gel; eluting with 0˜50% EtOAc in PE) to afford compound 16-f (298 mg, 37%) as a yellow solid. ¹H NMR (400 MHz, CD₃OD): δ 8.34 (t, 1H), 8.10 (s, 1H), 8.08 (dd, 1H), 7.86 (d, 1H), 7.55 (t, 1H), 5.21 (q, 1H), 2.38 (s, 3H), 1.65 (s, 9H), 1.62 (d, 3H); LCMS Mass: 399.2 (M⁺+H).

### Step 6: Synthesis of tert-butyl (S)-5-((1-(3-aminophenyl)ethyl)amino)-3-methyl-1H-pyrazolo[3,4-b]pyrazine-1-carboxylate (16-g)

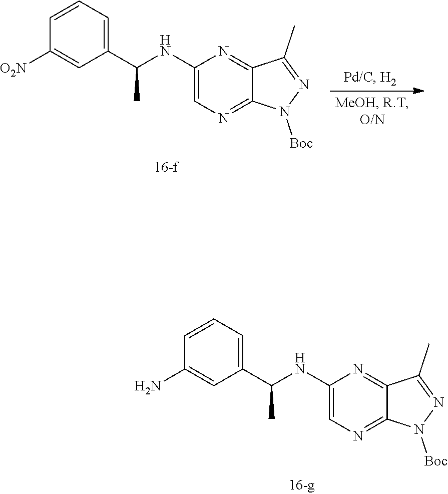 [SMILES: Cc1nn(C(=O)OC(C)(C)C)c2ncc(N[C@@H](C)c3cccc([N+](=O)[O-])c3)nc12 ; Cc1nn(C(=O)OC(C)(C)C)c2ncc(N[C@@H](C)c3cccc(N)c3)nc12]

To a stirred solution of 16-f (298 mg, 0.75 mmol) in methanol (20 mL), was added Pd/C (30 mg) and the mixture was stirred at r.t for 16 h under a H2 atmosphere. The reaction mixture was filtered through Celite and the filter cake was washed with methanol (20 mL). The filtrate was concentrated to dryness under reduced pressure to afford compound 16-g (250 mg, 91%) as a yellow solid, which was used directly without further purification. LCMS Mass: 368.2 (M⁺+H).

### Step 7: Synthesis of tert-butyl (S)-3-methyl-5-((1-(3-(5-methylnicotinamido)phenyl) ethyl)amino)-1H-pyrazolo[3,4-b]pyrazine-1-carboxylate (16-h)

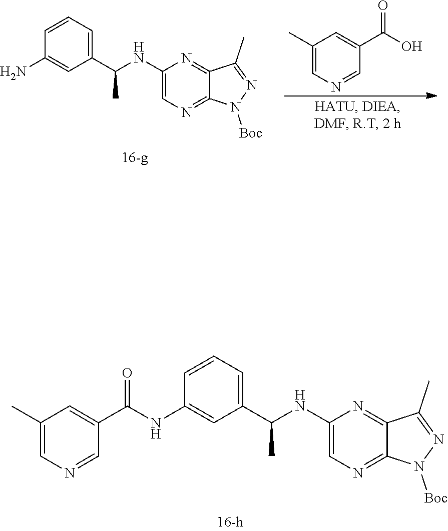

To a stirred solution of 5-methylnicotinic acid (44.6 mg, 0.32 mmol) in DMF (5 mL), was added HATU (155 mg, 0.41 mmol) and the mixture was stirred at r.t for 20 min. Compound 16-g (100 mg, 0.27 mmol) and DIPEA (105 mg, 0.81 mmol) were added and the mixture stirred at r.t for 16 h. The reaction mixture was partitioned between water (50 mL) and EtOAc (20 mL). The organic layer was separated and washed by brine (30 mL×2). The organic layer was separated, dried (Na₂SO₄), filtered, and was concentrated under reduced pressure. The crude residue was purified (silica gel; eluting with 0˜100% EtOAc in PE) to afford compound 16-h (96 mg, 73%) as a yellow oil. LCMS Mass: 488.30 (M⁺+H).

### Step 8: Synthesis of the hydrochloride salt of (S)-5-methyl-N-(3-(1-((3-methyl-1H-pyrazolo[3,4-b]pyrazin-5-yl)amino)ethyl)phenyl)nicotinamide (Compound 16)

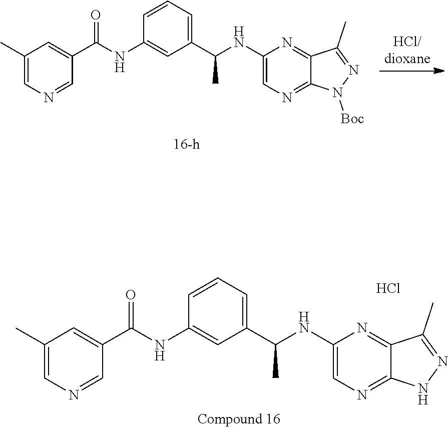 [SMILES: Cc1cncc(C(=O)Nc2cccc([C@H](C)Nc3cnc4c(n3)c(C)nn4C(=O)OC(C)(C)C)c2)c1 ; Cc1cncc(C(=O)Nc2cccc([C@H](C)Nc3cnc4[nH]nc(C)c4n3)c2)c1 ; Cl]

To a stirred solution of 16-h (96 mg, 13.5 mmol) in DCM (40 mL), was added 4 M HCl in 1,4-dioxane (4 mL) and the mixture was stirred at r.t for 16 h. The mixture was evaporated under reduced pressure and the crude residue was purified (Preparative Reverse-Phase HPLC; eluting with 0.1% HCl in H₂O/MeOH) to afford the hydrochloride salt of Compound 16 (38 mg, 49%) as a solid. ¹H NMR (400 MHz, DMSO-d₆): δ 10.86 (s, 1H), 9.22 (s, 1H), 8.89 (s, 1H), 8.77 (s, 1H), 8.12 (s, 1H), 7.87 (s, 1H), 7.69-7.67 (d, 2H), 7.34 (t, 1H), 7.27 (d, 1H), 5.23 (q, 1H), 2.52 (s, 3H), 2.36 (s, 3H), 1.53-1.51 (d, 3H); LCMS Mass: 388.2 (M⁺+H).

### Example 17

### Synthesis of the hydrochloride salt of (5)-5-methyl-N-(3-(1-((6-methylfuro[2,3-b] pyrazin-3-yl)amino)ethyl)phenyl)nicotinamide (Compound 17)

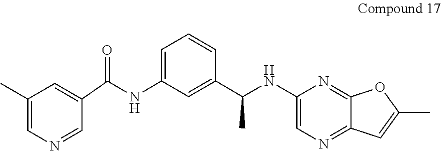

### Step 1: Synthesis of (R)-1-(3-nitrophenyl)ethyl methanesulfonate (17-b)

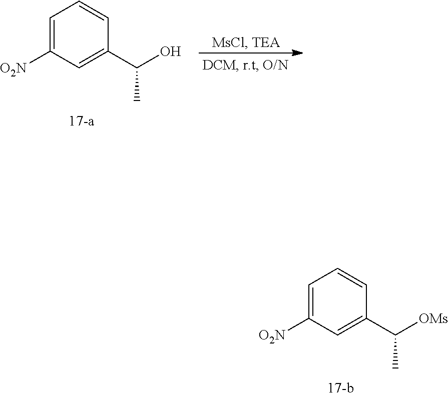 [SMILES: C[C@@H](O)c1cccc([N+](=O)[O-])c1 ; C[C@@H](OS(C)(=O)=O)c1cccc([N+](=O)[O-])c1]

To a stirred solution of (R)-1-(3-nitrophenyl)ethan-1-ol (17-a) (5.0 g, 29.91 mmol) in DCM (50 mL) at 0° C., was added methyl sulfonyl chloride (6.85 g, 59.82 mmol) and TEA (9.08 g, 89.73 mmol). The mixture was allowed to warm to r.t. and stirred for a further 16 h. The mixture was diluted with DCM (100 mL) and washed by water (200 mL), then brine (100 mL). The organic layer was separated, dried (Na₂SO₄), filtered, and concentrated under reduced pressure. The crude residue was purified (silica gel; eluting with 0˜20% EtOAc in PE) to afford compound 17-b (4.7 g, 64%) as a yellow oil, which was not purified further.

### Step 2: Synthesis of 6-chloro-5-(prop-1-yn-1-yl)pyrazin-2-amine (17-d)

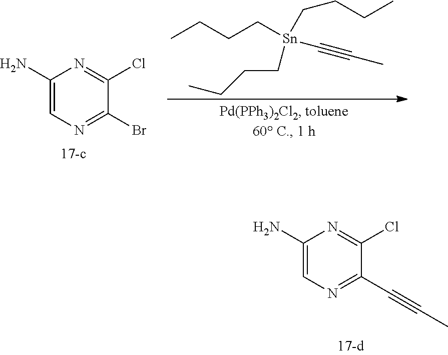

To a stirred solution of 5-bromo-6-chloropyrazin-2-amine (17-c) (4.8 g, 23.03 mmol) in DMF (50 mL), was added tributyl(prop-1-yn-1-yl)stannane (9.09 g, 27.64 mmol), Pd(PPh₃)₂Cl₂ (8.08 g, 11.51 mmol), CuI (2.19 g, 11.51 mmol) and TEA (6.99 g, 69.08 mmol) and the mixture was stirred at 90° C. for 16 h under a N₂ atmosphere. The reaction mixture was partitioned between water (250 mL) and EtOAc (150 mL). The organic layer was separated and washed by brine (200 mL×2). The organic layer was separated, dried (Na₂SO₄), filtered, and was concentrated under reduced pressure. The crude residue was purified (silica gel; eluting with 0˜20% EtOAc in PE) to afford compound 17-d (96 mg, 73%) as yellow solid. ¹H NMR (400 MHz, CDCl₃): δ 7.83 (s, 1H), 3.39 (s, 2H), 2.13 (s, 3H); LCMS Mass: 168.05 (M⁺+H).

### Step 3: Synthesis of 6-methylfuro[2,3-b]pyrazin-3-amine (17-e)

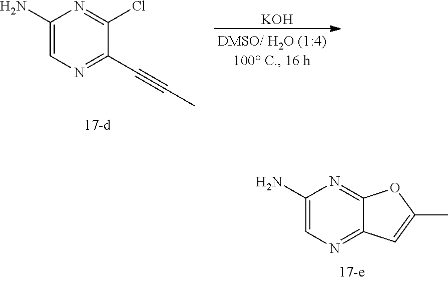

To a stirred solution of 17-d (1.77 g, 10.56 mmol) in DMSO/H₂O (30 mL), was added potassium hydroxide (1.19 g, 21.12 mmol) and the mixture was stirred at 100° C. for 16 h. The reaction mixture was partitioned between water (120 mL) and EtOAc (60 mL). The organic layer was separated and washed by brine (80 mL×2). The organic layer was separated, dried (Na₂SO₄), filtered, and was concentrated under reduced pressure. The crude residue was purified (silica gel; eluting with 0˜30% EtOAc in PE), to afford compound 17-e (871 mg, 55%) as yellow solid. ¹H NMR (400 MHz, CDCl₃): δ 7.91 (s, 1H), 6.48 (m, 1H), 2.47 (s, 3H); LCMS Mass: 150.10 (M⁺+H).

### Step 4: Synthesis of (S)-6-methyl-N-(1-(3-nitrophenyl)ethyl)furo[2,3-b]pyrazin-3-amine (17-f)

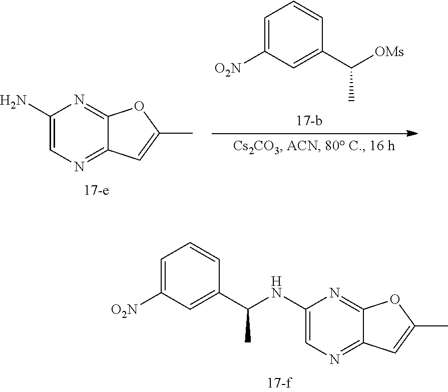 [SMILES: Cc1cc2ncc(N)nc2o1 ; C[C@@H](OS(C)(=O)=O)c1cccc([N+](=O)[O-])c1 ; Cc1cc2ncc(N[C@@H](C)c3cccc([N+](=O)[O-])c3)nc2o1]

To a stirred solution of 17-e (871 mg, 5.83 mmol) in acetonitrile (20 mL) was added (R)-1-(3-nitrophenyl)ethyl methanesulfonate (17-b) (2.58 g, 10.50 mmol), and Cs₂CO₃ (5.70 g, 17.50 mmol). The mixture was heated to reflux overnight. The reaction mixture was concentrated and the residue was dissolved in EtOAc (20 mL). The mixture was washed by water (50 mL) and brine (50 mL). The organic layer was separated, dried (Na₂SO₄), filtered, and concentrated under reduced pressure. The crude residue was purified (silica gel; eluting with 0˜20% EtOAc in PE), to afford compound 17-f (156 mg, 9%) as yellow solid.

### Step 5: Synthesis of (S)—N-(1-(3-aminophenyl)ethyl)-6-methylfuro[2,3-b]pyrazin-3-amine (17-g)

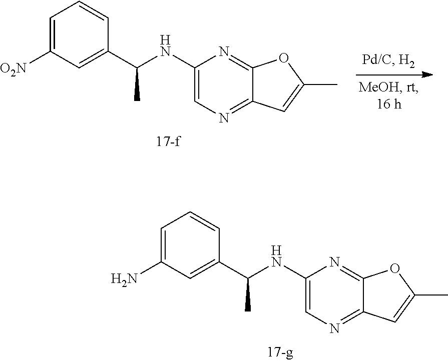 [SMILES: Cc1cc2ncc(N[C@@H](C)c3cccc([N+](=O)[O-])c3)nc2o1 ; Cc1cc2ncc(N[C@@H](C)c3cccc(N)c3)nc2o1]

To a stirred solution of 17-f (156 mg, 0.523 mmol) in methanol (10 mL), was added Pd/C (15.6 mg) and the mixture was stirred at r.t for 16 h under a H2 atmosphere. The reaction mixture was filtered by celite and the filter cake was washed by methanol (10 mL). The filtrate was concentrated to dryness under reduced pressure to afford compound 17-g (110 mg, 86%) as yellow solid. LCMS Mass: 269.20 (M⁺+H).

### Step 6: Synthesis of the hydrochloride salt of (S)-5-methyl-N-(3-(1-((6-methylfuro[2,3-b] pyrazin-3-yl)amino)ethyl)phenyl)nicotinamide (Compound 17)

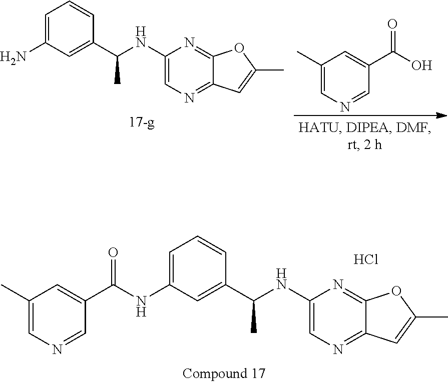

To a stirred solution of 5-methylnicotinic acid (67 mg, 0.491 mmol) in DMF (2 mL), was added HATU (233.82 mg, 0.614 mmol) and the mixture was stirred at r.t for 20 min. Compound 17-g (110 mg, 0.409 mmol) and DIPEA (166.75 mg, 0.819 mmol) were added and the mixture stirred at r.t for 2 h. The reaction mixture was partitioned between water (40 mL) and EtOAc (20 mL). The organic layer was separated and washed by brine (20 mL×2). The organic layer was separated, dried (Na₂SO₄), filtered, and was concentrated under reduced pressure. The crude residue was purified (Preparative Reverse-Phase HPLC; eluting with 0.1% HCl in H₂O/MeOH) to afford the hydrochloride salt of Compound 17 (113 mg, 72%) as a solid. ¹H NMR (400 MHz, DMSO-d₆): δ 10.72 (s, 1H), 9.16 (s, 1H), 8.85 (d, 1H), 8.66 (s, 1H), 7.91 (s, 1H), 7.77 (t, 1H), 7.65 (d, 1H), 7.32 (d, 1H), 7.19 (d, 1H), 6.53 (d, 1H), 4.92 (q, 1H), 2.50 (s, 3H), 2.34 (d, 3H), 1.49-1.47 (d, 3H); LCMS Mass: 388.25 (M⁺+H).

### Example 18

### Synthesis of the hydrochloride salt of (S)-5-methyl-N-(3-(1-((2-methylthieno[3,2-b] pyridin-6-yl)amino)ethyl)phenyl)nicotinamide (Compound 18)

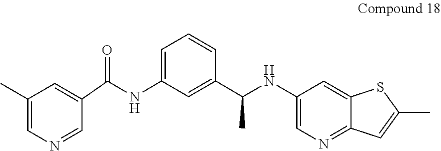

### Step 1: Synthesis of 5-methylthiophen-3-amine hydrochloride (18-b)

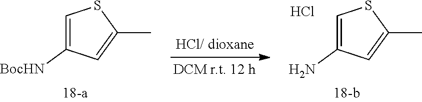

To a stirred solution of tert-butyl (5-methylthiophen-3-yl)carbamate (18-a) (1.00 g, 0.004 mol) in DCM was added HCl in 1,4-dioxane (5 ml). The reaction was stirred at room temperature overnight. The reaction was concentrated directly to afford 5-methylthiophen-3-amine hydrochloride (750 mg, 74%) as a white solid (18-b), which was not purified further.

### Step 2: Synthesis of 6-bromo-2-methylthieno[3,2-b]pyridine (18-c)

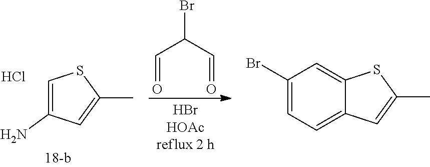

To a mixture of 18-b (100 mg, 0.671 mmol) and 2-bromomalonaldehyde (300 mg, 2.01 mmol) in HOAc (8 mL), was added HBr (2 mL) and PPh₃. The mixture was stirred at 130° C. overnight. After cooling to room temperature, the mixture was diluted with water and extracted with DCM (20 mL×3). The combined organic layers were dried (Na₂SO₄), filtered, and concentrated under reduced pressure to afford compound 18-c (75 mg, 50%) as yellow oil. ¹H NMR (400 MHz, CD₃OD) δ 8.57 (s, 1H), 8.47 (s, 1H), 7.18 (s, 1H), 2.65 (s, 3H).

### Step 3: Synthesis of the hydrochloride salt of (S)-5-methyl-N-(3-(1-((2-methylthieno[3,2-b]pyridin-6-yl)amino)ethyl)phenyl)nicotinamide (Compound 18)

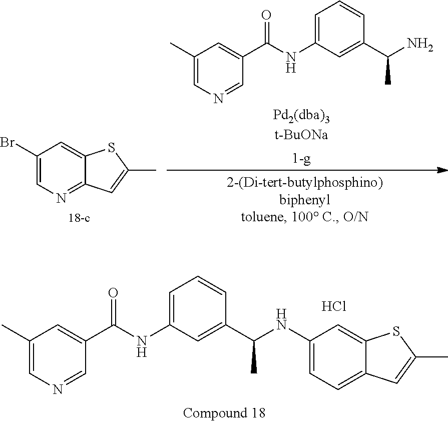

To a stirred solution of 18-c (75 mg, 0.711 mmol) and (S)—N-(3-(1-aminoethyl)phenyl)-5-methylnicotinamide (1-g) (prepared as described in Example 1, Step 6) (70 mg, 0.274 mmol) in toluene (5 mL), was added Pd₂(dba)₃ (25 mg, 0.027), 2-(di-tert-butylphosphino)biphenyl (8.1 mg, 0.027 mmol) and t-BuONa (80 mg, 0.822 mmol). The mixture was heated to 100° C. overnight under a N₂ atmosphere. The reaction mixture was partitioned between brine (40 mL) and EtOAc (20 mL×3). The combined organic layers were separated, dried (Na₂SO₄), filtered, and concentrated under reduced pressure. The crude residue was purified (Preparative Reverse-Phase HPLC; eluting with 0.1% HCl in H₂O/MeOH) to afford the hydrochloride salt of Compound 18 (20 mg, 15%) as a solid. ¹H NMR (400 MHz, CD₃OD) δ 9.22 (s, 1H), 9.02 (s, 1H), 8.90 (s, 1H), 8.02 (d, J=8.3 Hz, 2H), 7.92 (s, 1H), 7.66 (d, J=7.5 Hz, 1H), 7.39 (t, J=7.4 Hz, 1H), 7.30 (d, J=7.3 Hz, 1H), 7.18 (s, 1H), 4.74-4.66 (m, 1H), 2.67 (s, 3H), 2.64 (s, 3H), 1.63 (d, J=5.3 Hz, 3H); LCMS Mass: 403.20 (M⁺+H).

### Example 19

### Synthesis of the hydrochloride salt of (S)-5-methyl-N-(3-(1-((6-methylthieno[2,3-b] pyrazin-3-yl)amino)ethyl)phenyl)nicotinamide (Compound 19)

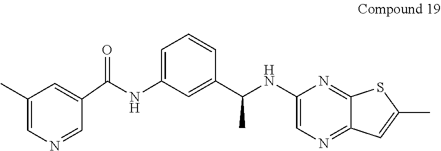

### Step 1: Synthesis of 6-methylthieno[2,3-b]pyrazin-3-amine (19-a)

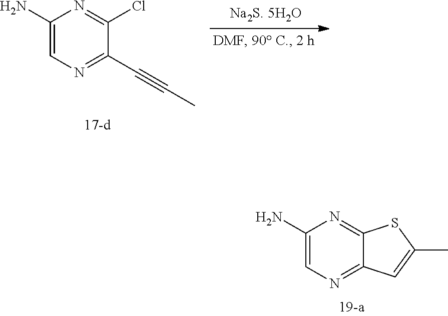

To a stirred solution of compound 17-d (prepared as described in Example 17, Step 2) (1.5 g, 8.95 mmol) in DMF (30 mL), was added sodium sulfide pentahydrate (6.02 g, 35.8 mmol) and the mixture was stirred at 90° C. for 16 h. The reaction mixture was partitioned between water (150 mL) and EtOAc (80 mL). The organic layer was separated and washed by brine (100 mL×2). The organic layer was separated, dried (Na₂SO₄), filtered, and was concentrated under reduced pressure. The crude residue was purified (silica gel; eluting with 0˜40% EtOAc in PE) to afford compound 19-a (790 mg, 53%) as a yellow solid. ¹H NMR (400 MHz, CDCl₃): δ 7.97 (s, 1H), 6.96 (d, 1H), 2.56 (d, 3H); LCMS Mass: 166.05 (M⁺+H).

### Step 2: Synthesis of (S)-6-methyl-N-(1-(3-nitrophenyl)ethyl)thieno[2,3-b]pyrazin-3-amine (19-b)

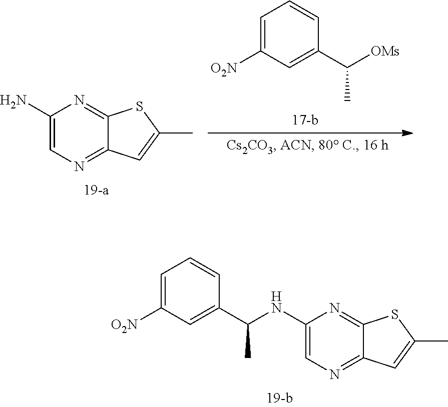 [SMILES: Cc1cc2ncc(N)nc2s1 ; C[C@@H](OS(C)(=O)=O)c1cccc([N+](=O)[O-])c1 ; Cc1cc2ncc(N[C@@H](C)c3cccc([N+](=O)[O-])c3)nc2s1]

To a stirred solution of 19-a (790 mg, 4.78 mmol) in acetonitrile (20 mL) was added 17-b (prepared as described in Example 17, Step 1) (2.35 g, 9.56 mmol) and Cs₂CO₃ (3.12 mg, 9.56 mmol). The mixture was heated to reflux for overnight. The reaction mixture was concentrated and the residue was dissolved in EtOAc (20 mL). The mixture was washed with water (50 mL) and then brine (50 mL). The organic layer was separated, dried (Na₂SO₄), filtered, and concentrated under reduced pressure. The crude residue was purified (silica gel; eluting with 0˜20% EtOAc in PE) to afford compound 19-b (160 mg, 11%) as a yellow oil. LCMS Mass: 315.10 (M⁺+H).

### Step 3: Synthesis of (S)—N-(1-(3-aminophenyl)ethyl)-6-methylthieno[2,3-b]pyrazin-3-amine (19-c)

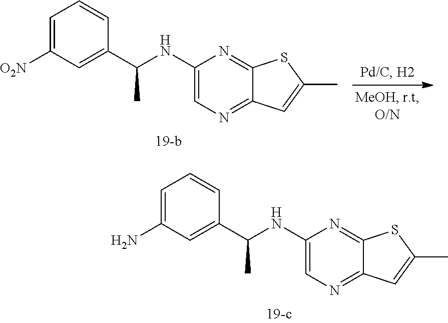 [SMILES: Cc1cc2ncc(N[C@@H](C)c3cccc([N+](=O)[O-])c3)nc2s1 ; Cc1cc2ncc(N[C@@H](C)c3cccc(N)c3)nc2s1]

To a stirred solution of 19-b (135 mg, 0.43 mmol) in methanol (10 mL), was added Pd/C (13.5 mg) and the mixture was stirred at r.t for 16 h under a H2 atmosphere. The reaction mixture was filtered through Celite and the filter cake was washed by methanol (10 mL). The filtrate was concentrated to dryness under reduced pressure to afford compound 19-c (120 mg, 98%) as a yellow solid. The crude was used directly without further purification. LCMS Mass: 285.15 (M⁺+H).

### Step 4: Synthesis of the hydrochloride salt of (S)-5-methyl-N-(3-(1-((6-methylthieno[2,3-b]pyrazin-3-yl)amino)ethyl)phenyl)nicotinamide (Compound 19)

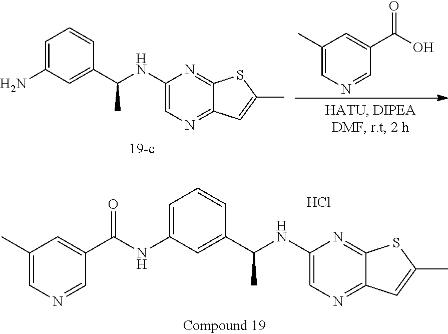

To a stirred solution of 5-methylnicotinic acid (69 mg, 051 mmol) in DMF (5 mL), was added HATU (240 mg, 0.63 mmol) and the mixture was stirred at r.t for 20 min. Compound 19-c (120 mg, 0.42 mmol) and DIPEA (109 mg, 0.84 mmol) were added and the mixture stirred at r.t for 2 h. The reaction mixture was partitioned between water (50 mL) and EtOAc (20 mL). The organic layer was separated and washed by brine (20 mL×2). The organic layer was separated, dried (Na₂SO₄), filtered, and was concentrated under reduced pressure. The crude residue was purified (Preparative Reverse-Phase HPLC; eluting with 0.1% HCl in H₂O/MeOH) to afford the hydrochloride salt of Compound 19 (63 mg, 37%) as a solid. ¹H NMR (400 MHz, DMSO-d6): δ 10.86 (s, 1H), 9.23 (s, 1H), 8.91 (s, 1H), 8.80 (s, 1H), 8.02 (s, 1H), 7.80 (s, 1H), 7.69 (d, 1H), 7.33 (t, 1H), 7.21 (d, 1H), 6.95 (d, 1H), 5.04 (q, 1H), 2.52 (s, 3H), 2.46-2.45 (d, 3H), 1.50-1.38 (d, 3H). LCMS Mass: 404.20 (M⁺+H).

### Example 20

### Synthesis of the hydrochloride salt of (5)-5-methyl-N-(3-(1-(pyrazolo[1,5-a]pyridin-3-yl) amino)ethyl)phenyl)nicotinamide (Compound 20)

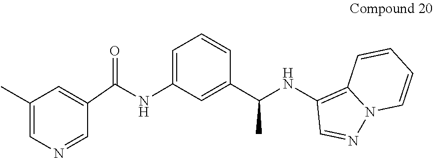 [SMILES: Cc1cncc(C(=O)Nc2cccc([C@H](C)Nc3cnn4ccccc34)c2)c1]

### Step 1: Synthesis of (R)-1-(3-aminophenyl)ethan-1-ol (20-b)

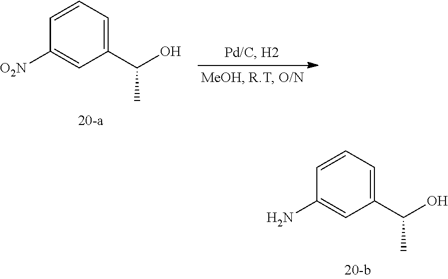

To a stirred solution of (R)-1-(3-nitrophenyl)ethan-1-ol (20-a) (3.00 g, 17.0 mmol) in methanol (30 mL), was added Pd/C (400 mg) and the mixture was stirred at r.t for 16 h under H₂ (1 atmosphere). The reaction mixture was filtered through Celite and the filter cake was washed with methanol (20 mL). The filtrate was concentrated to dryness under reduced pressure to afford compound 20-b (2.43 g, 98%) as brown oil. The crude was used directly without further purification. ¹H NMR (400 MHz, CDCl₃) δ 7.13-7.10 (t, 1H), 6.74-6.73 (d, 1H), 6.70 (s, 1H), 6.59-6.58 (t, 1H), 4.80-4.76 (q, 1H), 2.81 (br m, 3H), 1.46-1.44 (d, 3H); LCMS Mass: 138.2 (M⁺+H).

### Step 2: Synthesis of (R)—N-(3-(1-hydroxyethyl)phenyl)-5-methylnicotinamide (20-c)

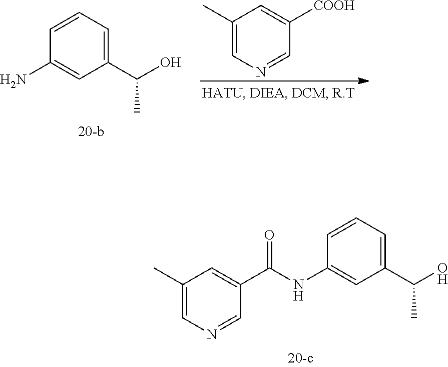

To a mixture of 5-methylnicotinic acid (2.67 g, 19.4 mmol) in DCM (50 mL), was added HATU (7.40 g, 19.4 mmol) and the mixture was stirred at r.t for 20 min. Compound 20-b (2.43 g, 17.0 mmol) and DIPEA (4.57 g, 35.4 mmol) were added and the mixture stirred at r.t for 16 h. The reaction mixture was partitioned between water (200 mL) and DCM (120 mL). The organic layer was separated and washed by brine (100 mL×2). The combined organic layers were dried (Na₂SO₄), filtered, and concentrated under reduced pressure. The crude residue was purified (silica gel; eluting with 0˜50% EtOAc in PE) to afford compound 20-c (4.17 g, 92%) as a yellow solid. ¹H NMR (400 MHz, DMSO-d₆) δ 10.35 (s, 1H), 8.91 (s, 1H), 8.60 (s, 1H), 8.12 (s, 1H), 7.75 (s, 1H), 7.67-7.65 (d, 1H), 7.31-7.28 (t, 1H), 7.09-7.08 (d, 1H), 5.19 (m, 1H), 4.71 (m, 1H), 2.39 (s, 3H), 1.34 (d, 3H).

### Step 3: Synthesis of (R)-1-(3-(5-methylnicotinamido)phenyl)ethyl methanesulfonate (20-d)

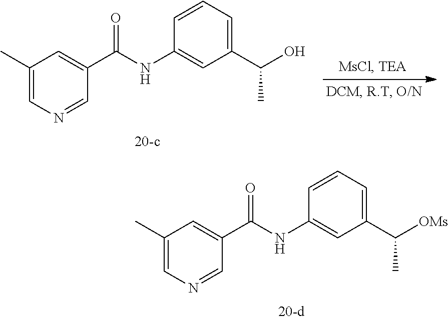 [SMILES: Cc1cncc(C(=O)Nc2cccc([C@@H](C)O)c2)c1 ; Cc1cncc(C(=O)Nc2cccc([C@@H](C)OS(C)(=O)=O)c2)c1]

To a stirred solution of 20-c (1.50 g, 5.85 mmol) in DCM (30 mL) at 0° C., was added methyl sulfonyl chloride (1.34 g, 11.7 mmol) and TEA (2.96 g, 29.26 mmol). The mixture was allowed to warm to r.t. and stirred for a further 16 h. The mixture was diluted with DCM (100 mL) and washed by water (200 mL), then brine (100 mL). The organic layer was separated, dried (Na₂SO₄), filtered, and concentrated under reduced pressure. The crude residue was purified (silica gel; eluting with 0˜50% EtOAc in PE) to afford compound 20-d (720 mg, 37%) as a yellow oil. ¹H NMR (400 MHz, CDCl₃): δ 9.08 (s, 1H), 8.57 (s, 1H), 8.15 (s, 1H), 7.82 (s, 1H), 7.67-7.64 (d, 1H), 7.37-7.33 (m, 3H), 5.11-5.06 (q, 1H), 2.70 (br s, 3H), 2.44 (s, 3H), 1.86-1.84 (d, 3H).

### Step 4: Synthesis of 1,1-diphenyl-N-(pyrazolo[1,5-a]pyridin-3-yl)methanimine (20-f)

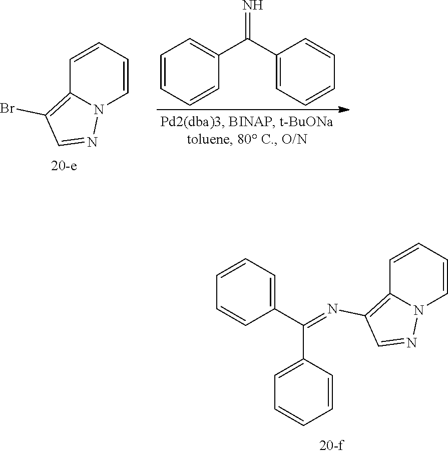

To a stirred solution of 3-bromopyrazolo[1,5-a]pyridine (20-e) (400 mg, 2.03 mmol) in toluene (15 mL), was added diphenylmethanimine (405 mg, 2.23 mmol), Pd₂(dba)₃ (186 mg, 0.20 mmol), BINAP (126 mg, 0.20 mmol), t-BuONa (585 mg, 6.09 mmol) and the mixture was stirred at 80° C. for 16 h under a N₂ atmosphere. The mixture was diluted with EtOAc (100 mL) and washed by water (200 mL), then brine (100 mL). The organic layer was separated, dried (Na₂SO₄), filtered, and concentrated under reduced pressure. The crude residue was purified (silica gel; eluting with 0˜20% EtOAc in PE) to afford compound 20-f (270 mg, 45%) as a yellow oil. ¹H NMR (400 MHz, DMSO-d₆): δ 7.52-7.51 (d, 1H), 7.11-7.09 (d, 1H), 6.99-6.98 (d, 2H), 6.75-6.74 (d, 3H), 6.61-6.56 (q, 3H), 6.47-6.44 (t, 3H), 6.09-6.06 (t, 1H), 5.62 (s, 1H); LCMS Mass: 298.1 (M+H⁺).

### Step 5: Synthesis of pyrazolo[1,5-a]pyridin-3-amine hydrochloride (20-g)

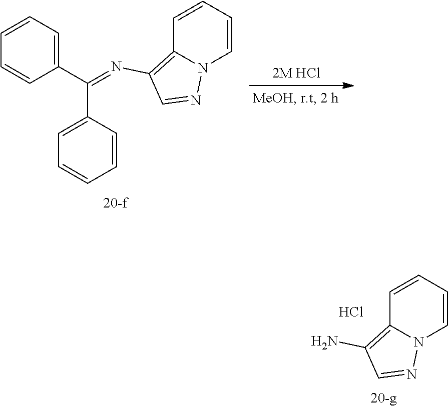 [SMILES: c1ccc(C(=Nc2cnn3ccccc23)c2ccccc2)cc1 ; Nc1cnn2ccccc12 ; Cl]

To a stirred solution of 20-f (270 mg, 0.90 mmol) in methanol (10 mL), was added 2M HCl aqueous (5 mL) and the mixture was stirred at r.t for 2 h. The mixture was concentrated to afford compound 20-g (183 mg) as red solid that was not further purified. LCMS Mass: 134.1 (M⁺+H).

### Step 6: Synthesis of the hydrochloride salt of (S)-5-methyl-N-(3-(1-(pyrazolo[1,5-a] pyridin-3-ylamino)ethyl)phenyl)nicotinamide (Compound 20)

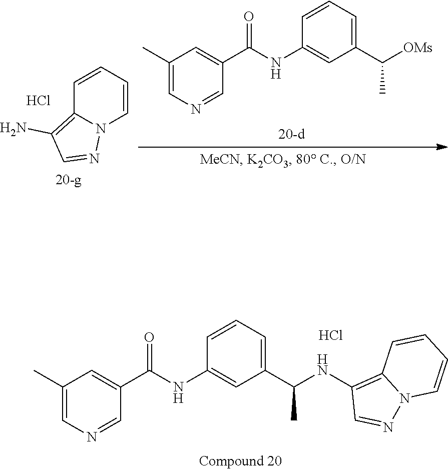

To a stirred solution of 20-g (84 mg, 0.63 mmol) in acetonitrile (5 mL) was added 20-d (316 mg, 0.94 mmol), K₂CO₃ (261 mg, 1.89 mmol). The mixture was heated to reflux overnight. The reaction mixture was concentrated and the residue was dissolved in EtOAc (20 mL). The mixture was washed with water (50 mL), then brine (50 mL). The organic layer was separated, dried (Na₂SO₄), filtered, and concentrated under reduced pressure. The crude residue was purified (Preparative Reverse-Phase HPLC; eluting with 0.1% HCl in H₂O/MeOH) to afford the hydrochloride salt of Compound 20 (15 mg, 6%) as a solid. ¹H NMR (400 MHz, CD₃OD): δ 9.23 (s, 1H), 9.03 (s, 1H), 8.92 (s, 1H), 8.55-8.54 (d, 1H), 8.02 (s, 1H), 7.92 (s, 1H), 7.76-7.74 (d, 1H), 7.50-7.48 (d, 1H), 7.42-7.38 (t, 1H), 7.33-7.29 (t, 1H), 7.21-7.19 (d, 1H), 7.00-6.97 (t, 1H), 4.88 (s, 1H), 2.68 (s, 3H), 1.89-1.87 (d, 3H); LCMS Mass: 372.2 (M⁺+H).

### Example 21

### Synthesis of (S)—N-(3-(1-((1-(1-methyl-1H-pyrazol-4-yl)-1H-pyrazolo[3,4-c]pyridin-4-yl) amino)ethyl)phenyl)nicotinamide (Compound 21)

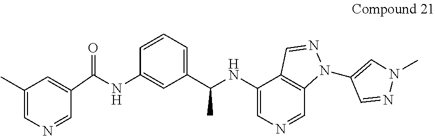

### Step 1: Synthesis of 4-bromo-1-(1-methyl-1H-pyrazol-4-yl)-1H-pyrazolo[3,4-c]pyridine (21-d)

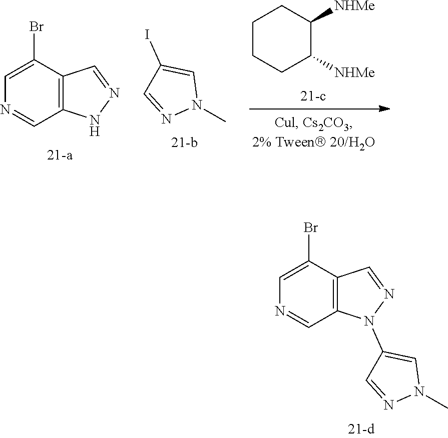

To a stirred solution of 4-bromo-2H-pyrazolo[3,4-c]pyridine (21-a) (250 mg, 1.27 mmol) in 2% Tween® 20/H₂O (3 mL) at rt, was added 4-iodo-1-methyl-(21-b) (317 mg, 1.52 mmol), CuI (161 mg, 0.254 mmol), Cs₂CO₃ (1.03 g, 3.17 mmol), and compound 21-c (181 mg, 1.27 mmol). The mixture was heated to 60° C. under N₂ for 2 h. The mixture was diluted with water and extracted with EtOAc. The organic layer was washed with saturated aq. NH₄Cl and then dried (MgSO₄), filtered, and concentrated under reduced pressure. The crude residue was purified (silica gel; eluting with 0-100% EtOAc in heptane) to afford compound 21-d (120 mg, 34%). LCMS Mass: 279.98 (M⁺+H).

### Step 2: Synthesis of (S)—N-(3-(1-((1-(1-methyl-1H-pyrazol-4-yl)-1H-pyrazolo[3,4-c] pyridin-4-yl)amino)ethyl)phenyl)nicotinamide (Compound 21)

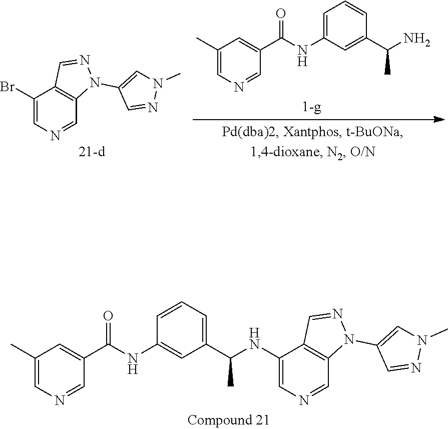

To a stirred solution of compound 21-d (120 mg, 0.431 mmol) in 1,4-dioxane (5 mL) at rt, was added (S)—N-(3-(1-aminoethyl)phenyl)-5-methylnicotinamide (1-g) (prepared as described in Example 1, Step 6) (100 mg, 0.392 mmol), Pd(dba)2 (23 mg, 0.039 mmol), Xantphos (23 mg, 0.039 mmol) and t-BuONa (2M, 392 μL). The mixture was heated to reflux under N₂ for 2 h. The mixture was diluted with EtOAc and washed with saturated aq. NH₄Cl and brine. The organic phase was dried (MgSO₄), filtered, and concentrated under reduced pressure. The residue was purified (Preparative HPLC; eluting with 0.05% TFA in H2O/acetonitrile) to afford Compound 21 (25 mg, 11%). 1H NMR (DMSO-d6, 300 MHz) δ 10.42 (s, 1H), 8.8-9.0 (m, 2H), 8.5-8.8 (m, 2H), 8.42 (s, 1H), 7.9-8.3 (m, 4H), 7.62 (d, 1H, J=8.8 Hz), 7.2-7.4 (m, 3H), 4.94 (br t, 1H, J=6.6 Hz), 3.95 (s, 3H), 2.39 (s, 3H), 1.64 (d, 3H, J=6.6 Hz); LCMS Mass: 453.36 (M++H).

### Example 22

### Synthesis of the hydrochloride salt of (5)-5-methyl-N-(3-(1-((2-methylfuro[3,2-b]pyridin-6-yl)amino)ethyl)phenyl)nicotinamide (Compound 22)

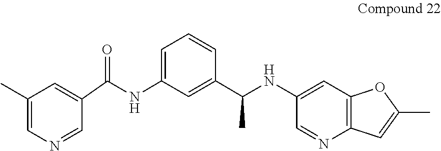

### Step 1: Synthesis of 6-bromo-2-methylfuro[3,2-b]pyridine (22-b)

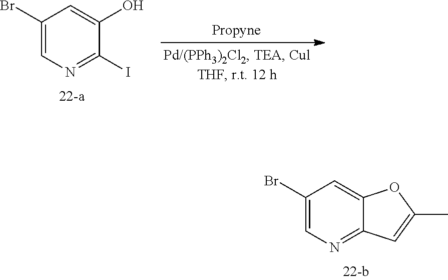

To a stirred solution of 5-bromo-2-iodopyridin-3-ol (22-a) (1.00 g, 0.003 mol) and TEA (1.01 g, 0.009 mol) in THF, was added Propyne (0.67 g, 0.015 mol), CuI (0.95 g, 0.005 mol) and Pd(PPh3)2Cl2 (25 mg, 0.0003 mol). The reaction mixture was stirred at room temperature under a nitrogen atmosphere overnight. The reaction was diluted with water, and extracted with EtOAc. The organic phase was washed with water and brine, then concentrated and purified by Prep-TLC to afford 6-bromo-2-methylfuro[3,2-b]pyridine (22-b) (412 mg, 58%) as a yellow oil.

### Step 2: Synthesis of the hydrochloride salt of (S)-5-methyl-N-(3-(1-((2-methylfuro[3,2-b] pyridin-6-yl)amino)ethyl)phenyl)nicotinamide (Compound 22)

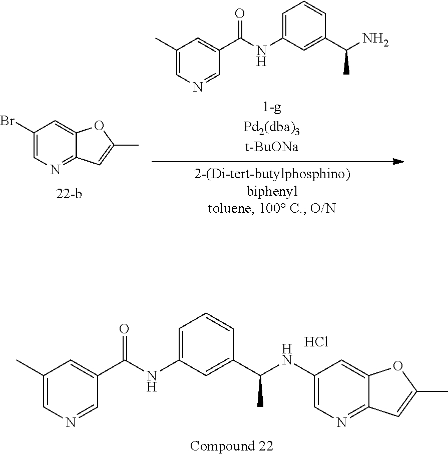

To a stirred solution of 22-b (150 mg, 0.711 mmol) and (S)—N-(3-(1-aminoethyl)phenyl)-5-methylnicotinamide (1-g) (prepared as described in Example 1, Step 6) (70 mg, 0.274 mmol) in toluene (5 mL) was added Pd₂(dba)₃ (25 mg, 0.027), 2-(di-tert-butylphosphino)biphenyl (8.1 mg, 0.027 mmol) and t-BuONa (80 mg, 0.822 mmol). The mixture was heated to 100° C. overnight under a nitrogen atmosphere. The reaction mixture was partitioned between brine (50 mL) and EtOAc (50 mL). The organic layer was separated, dried (Na₂SO₄), filtered, and concentrated under reduced pressure. The crude residue was purified (Preparative Reverse-Phase HPLC; eluting with 0.1% HCl in H₂O/MeOH) to afford the hydrochloride salt of Compound 22 (50 mg, 47%) as a solid. ¹H NMR (400 MHz, DMSO-d₆) δ 10.79 (s, 1H), 9.14 (s, 1H), 8.82 (s, 1H), 8.60 (s, 1H), 7.96 (s, 1H), 7.88 (s, 1H), 7.65 (d, J=8.9 Hz, 2H), 7.35 (t, J=7.8 Hz, 1H), 7.23 (d, J=7.7 Hz, 1H), 6.85 (s, 1H), 4.72-4.67 (m, 1H), 2.48-2.50 (2×s, 6H), 1.51 (d, J=6.6 Hz, 3H). LCMS 387.3 (M⁺+H).

### Example 23

### Synthesis of the hydrochloride salt of (S)—N-(3-(1-((1H-imidazo[4,5-b]pyrazin-5-yl) amino)ethyl)phenyl)-5-methylnicotinamide (Compound 23)

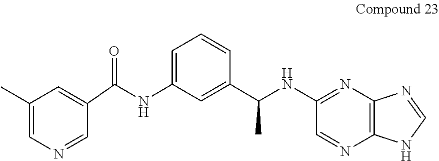 [SMILES: Cc1cncc(C(=O)Nc2cccc([C@H](C)Nc3cnc4[nH]cnc4n3)c2)c1]

### Step 1: Synthesis of 5-bromo-1-((2-(trimethylsilyl)ethoxy)methyl)-1H-imidazo[4,5-b] pyrazine (23-b)

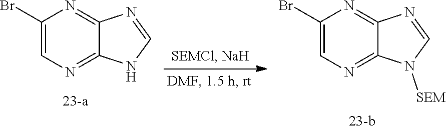 [SMILES: Brc1cnc2[nH]cnc2n1 ; C[Si](C)(C)CCOCn1cnc2nc(Br)cnc21]

To a stirred solution of 5-bromo-1H-imidazo[4,5-b]pyrazine (23-a) (300 mg, 1.515 mmol) and NaH (73 mg, 3.03 mmol) in DMF, was added SEMCl (505 mg, 3.03 mmol) and the mixture was stirred at r.t. for 1.5 h. The mixture was extracted with DCM (30 mL×3) and the combined organic layers were combined and dried (Na₂SO₄), filtered, and was concentrated under reduced pressure. The crude was purified (Preparative TLC; eluting with DCM:MeOH=20:1) to afford compound 23-b (320 mg, 64%) as a brown solid. ¹H NMR (400 MHz, CDCl₃) δ 8.46-8.41 (m, 2H), 5.64 (d, J=4.6 Hz, 2H), 3.60 (q, J=8.5 Hz, 2H), 0.91 (ddd, J=9.1, 7.7, 5.4 Hz, 2H), −0.06 (dd, J=4.3, 1.1 Hz, 9H); LCMS Mass: 329 (M⁺+H).

### Step 2: Synthesis of -5-methyl-N-(3-(1-((1-((2-(trimethylsilyl)ethoxy)methyl)-1H-imidazo[4,5-b]pyrazin-5-yl)amino)ethyl)phenyl)nicotinamide (23-c)

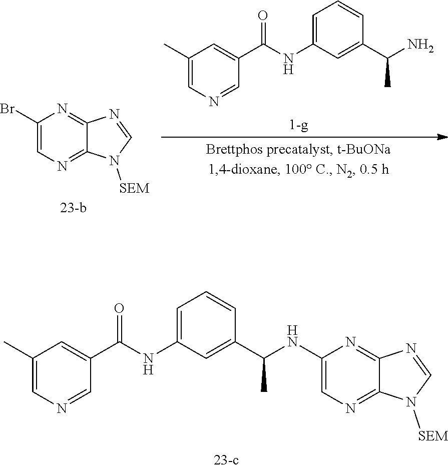 [SMILES: Cc1cncc(C(=O)Nc2cccc([C@H](C)N)c2)c1 ; C[Si](C)(C)CCOCn1cnc2nc(Br)cnc21 ; Cc1cncc(C(=O)Nc2cccc([C@H](C)Nc3cnc4c(ncn4COCC[Si](C)(C)C)n3)c2)c1]

To a stirred solution of 23-b (100 mg, 0.3 mmol) and (S)—N-(3-(1-aminoethyl)phenyl)-5-methylnicotinamide (1-g) (prepared as described in Example 1, Step 6) (85.6 mg, 0.335 mmol) in toluene, was added Pd₂(dba)₃ (27.5 mg, 0.03 mmol), BINAP (18.7 mg, 0.03 mmol) and K₃PO₄ (191 mg) and the mixture was stirred at 100° C. under a N₂ atmosphere overnight. The reaction mixture was partitioned between brine (20 mL) and EtOAc (20 mL). The organic layer was separated, dried (Na₂SO₄), filtered, and concentrated under reduced pressure. The crude residue was purified (Preparative TLC; eluting with DCM:MeOH=20:1) to afford afford Compound 23-c (91 mg, 60%) as a brown solid. LCMS Mass: 504.2 (M⁺+H).

### Step 3: Synthesis of the Hydrochloride Salt of (S)—N-(3-(1-((1H-imidazo[4,5-b]pyrazin-5-yl)amino)ethyl)phenyl)-5-methylnicotinamide (Compound 23)

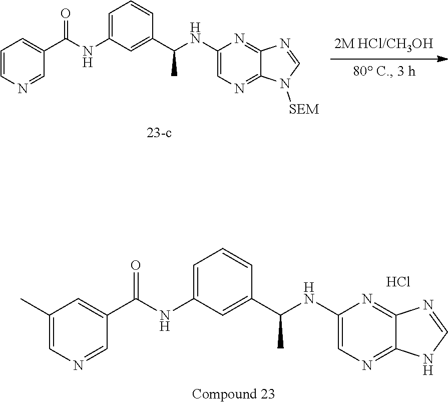 [SMILES: C[C@H](Nc1cnc2c(ncn2COCC[Si](C)(C)C)n1)c1cccc(NC(=O)c2cccnc2)c1 ; Cc1cncc(C(=O)Nc2cccc([C@H](C)Nc3cnc4[nH]cnc4n3)c2)c1 ; Cl]

To a solution of 23-c (91 mg, 0.18 mmol) in 4 M HCl (3 mL) and MeOH (3 mL) was stirred at 80° C. for 3 h. The mixture was concentrated and was purified (Preparative Reverse-Phase HPLC; eluting with 0.1% HCl in H₂O/MeOH) to afford the hydrochloride salt of Compound 23 (15 mg, 22%) as a yellow solid. ¹H NMR (400 MHz, CD₃OD) δ 9.22 (s, 1H), 9.13 (s, 1H), 9.01 (s, 1H), 8.90 (s, 1H), 8.14 (s, 1H), 7.90 (t, J=1.8 Hz, 1H), 7.64-7.59 (m, 1H), 7.35 (t, J=7.8 Hz, 1H), 7.27 (d, J=7.6 Hz, 1H), 5.13 (q, J=6.9 Hz, 1H), 2.67 (s, 3H), 1.62 (d, J=6.9 Hz, 3H); LCMS Mass: 374.2 (M⁺+H).

### Example 24

### Synthesis of the hydrochloride salt of ((S)—N-(3-(1-((2-ethyl-3H-imidazo[4,5-b]pyridin-6-yl)amino)ethyl)phenyl)-5-methylnicotinamide (Compound 24)

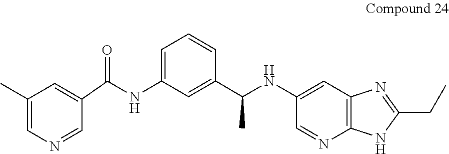 [SMILES: CCc1nc2cc(N[C@@H](C)c3cccc(NC(=O)c4cncc(C)c4)c3)cnc2[nH]1]

### Step 1: Synthesis of 6-bromo-2-ethyl-3H-imidazo[4,5-b]pyridine (24-b)

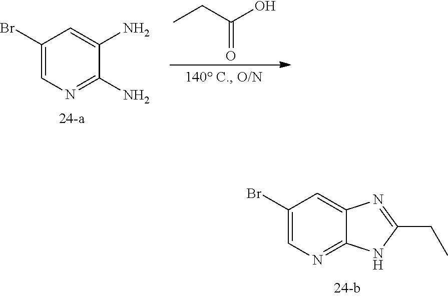

A solution of 5-bromopyridine-2,3-diamine (24-a) (500 mg, 2.66 mmol) in propionic acid (5 mL) was stirred at 140° C. overnight. The reaction mixture was concentrated and was purified by (silica gel; eluting with DCM:MeOH=20:1) to afford 24-b (390 mg, 65%) as a brown solid. ¹H NMR (400 MHz, Methanol-d₄) δ 8.35 (d, J=2.1 Hz, 1H), 8.07 (d, J=2.1 Hz, 1H), 2.96 (q, J=7.6 Hz, 2H), 1.41 (t, J=7.6 Hz, 3H); LCMS Mass: 226 (M⁺+H).

### Step 2: Synthesis of 6-bromo-2-ethyl-3-((2-(trimethylsilyl)ethoxy)methyl)-3H-imidazo [4,5-b]pyridine (24-c)

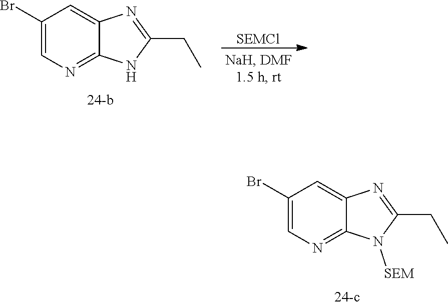 [SMILES: CCc1nc2cc(Br)cnc2[nH]1 ; "SEMCl" ; CCc1nc2cc(Br)cnc2n1COCC[Si](C)(C)C]

To a stirred solution of 24-b (200 mg, 0.89 mmol) and NaH (43 mg, 1.78 mg) in DMF (5 mL) at r.t., was added SEMCl (296.4 mg, 1.78 mmol) and the mixture was stirred at r.t. for 1.5 h. The mixture was extracted with DCM (30 mL×3). The combined organic layers were dried (Na₂SO₄), filtered, and concentrated under reduced pressure. The crude was purified (Preparative TLC; eluting with DCM:MeOH=20:1) to afford compound 24-c (180 mg, 57%) as a brown solid. ¹H NMR (400 MHz, Chloroform-d) δ 8.54 (d, J=2.1 Hz, 1H), 7.85 (d, J=2.1 Hz, 1H), 5.44 (s, 2H), 3.55-3.49 (m, 2H), 2.99-2.95 (q, J=7.5 Hz, 2H), 1.49 (t, J=7.5 Hz, 3H), 0.93-0.88 (m, 2H), −0.03 (s, 9H). LCMS Mass: 356 (M⁺+H).

### Step 3: Synthesis of (S)—N-(3-(1-((2-ethyl-3-((2-(trimethylsilyl)ethoxy)methyl)-3H-imidazo[4,5-b]pyridin-6-yl)amino)ethyl)phenyl)-5-methylnicotinamide (24-d)

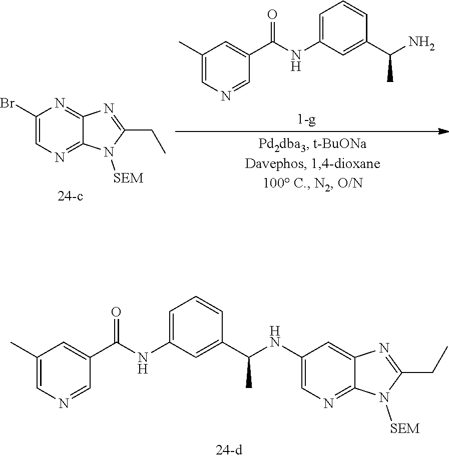 [SMILES: Cc1cncc(C(=O)Nc2cccc([C@H](C)N)c2)c1 ; CCc1nc2nc(Br)cnc2n1COCC[Si](C)(C)C ; CCc1nc2cc(N[C@@H](C)c3cccc(NC(=O)c4cncc(C)c4)c3)cnc2n1COCC[Si](C)(C)C]

To a stirred solution of 24-c (100 mg, 0.28 mmol) and (S)—N-(3-(1-aminoethyl)phenyl)-5-methylnicotinamide (1-g) (prepared as described in Example 1, Step 6) (79.1 mg, 0.31 mmol) in toluene was added Pd₂(dba)₃ (25.6 mg, 0.028 mmol), Jonhphos (8.36 mg, 0.028 mmol) and t-BuONa (in THF) (53.8 mg, 0.56 mmol). The mixture was stirred at 100° C. under a N₂ atmosphere overnight. The mixture was extracted with DCM (20 mL×3). The organic phase was combined and dried (Na₂SO₄), filtered, and concentrated under reduced pressure. The crude was purified (Preparative TLC; eluting with DCM:MeOH=20:1) to afford compound 24-d (45 mg, 30%) as a brown solid. LCMS Mass: 531.0 (M⁺+H).

### Step 4: Synthesis of the hydrochloride salt of ((S)—N-(3-(1-((2-ethyl-3H-imidazo[4,5-b] pyridin-6-yl)amino)ethyl)phenyl)-5-methylnicotinamide (Compound 24)

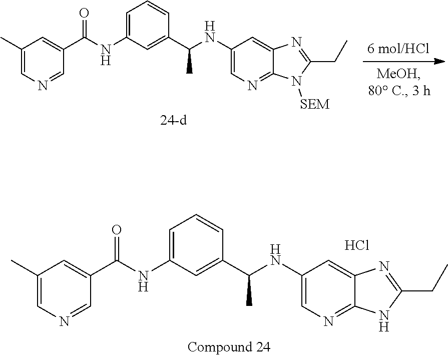

A stirred solution of 24-d (45 mg, 0.085 mmol), 6M HCl (2 mL), and MeOH was heated at 80° C. for 3 h. The mixture was concentrated under reduced pressure and the residue was purified (Preparative Reverse-Phase HPLC; eluting with 0.1% HCl in H₂O/MeOH) to afford the hydrochloride salt of Compound 24 (5 mg, 15%) as a yellow solid. ¹H NMR (400 MHz, Methanol-d₄) δ 9.11 (s, 1H), 8.91 (s, 1H), 8.80 (s, 1H), 8.03 (d, J=2.3 Hz, 1H), 7.79 (t, J=1.8 Hz, 1H), 7.55 (dd, J=7.8, 1.8 Hz, 1H), 7.28 (t, J=7.8 Hz, 1H), 7.18 (d, J=7.6 Hz, 1H), 6.97 (d, J=2.4 Hz, 1H), 4.51 (q, J=6.7 Hz, 1H), 2.99 (q, J=7.6 Hz, 2H), 2.57 (s, 3H), 1.53 (d, J=6.6 Hz, 3H), 1.33 (t, J=7.5 Hz, 3H); LCMS Mass: 401.3 (M⁺+H).

### Example 25

### Synthesis of (S)—N-(3-(1-((2-ethyl-2H-pyrazolo[3,4-b]pyrazin-6-yl)amino)ethyl)phenyl)-2-(5-methylpyridin-2-yl)acetamide (Compound 25)

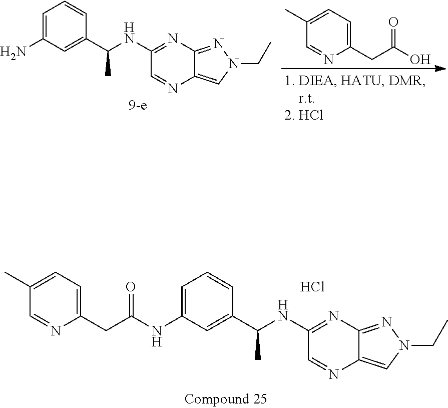

To a stirred solution of 2-(5-methyl-2-pyridyl)acetic acid (27 mg, 0.177 mmol) in DMA (1 mL) at rt, was added HATU (74 mg, 0.195 mmol) and DIPEA (35 mg, 46.8 mmol). The mixture was stirred at rt for 10 min. Compound 9-e (prepared as described in Example 9, Step 3) (50 mg, 0.177 mmol) was added and the reaction was stirred at rt for 16 h. The mixture was purified (Preparative HPLC; eluting with 0.1% FA in H₂O/acetonitrile) to give an off-white solid after lyophilization. The solid was dissolved in acetonitrile (2 mL) and 4 M HCl in 1,4-dioxane (0.186 mmol, 8.5 uL) was added. The reaction was stirred at rt for 1 h and lyophilized to afford Compound 25 (40 mg, 50%) as a light yellow solid. ¹H NMR (300 MHz, DMSO-d6) δ ppm 10.44 (s, 1H) 8.68 (s, 1H) 8.32 (s, 1H) 8.21 (br d, J=7.24 Hz, 1H) 8.03 (s, 1H) 7.97 (br d, J=6.79 Hz, 1H) 7.78 (d, J=8.16 Hz, 1H) 7.61 (s, 1H) 7.47 (br d, J=8.62 Hz, 1H) 7.27 (t, J=7.93 Hz, 1H) 7.12 (d, J=7.52 Hz, 1H) 5.04 (quin, J=6.79 Hz, 1H) 4.22 (q, J=7.40 Hz, 2H) 4.11 (s, 2H) 2.43 (s, 3H) 1.47 (d, J=6.97 Hz, 3H) 1.41 (t, J=7.24 Hz, 3H); LCMS 416.4 (M⁺+H).

### Examples 26-751

Compound Nos. 2-751 listed in Table 6 below were prepared according to the methods described in Schemes 1 through 3 and Examples 1 through 25, as shown above, using the appropriately substituted or modified intermediates.

TABLE 6
COMPOUNDS PREPARED ACCORDING TO SYNTHETIC SCHEMES 1 THROUGH 3

| | | | Observed |
|---|---|---|---|
| Cmpd | | | Mass |
| No | Structure | MW | (LCMS m/z) |
| 26 | 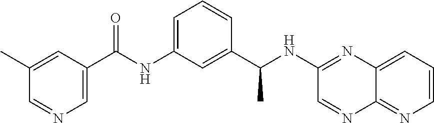 [SMILES: Cc1cncc(C(=O)Nc2cccc([C@H](C)Nc3cnc4ncccc4n3)c2)c1] | 384.43 | 385.19 |
| 27 | 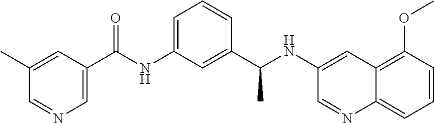 | 412.48 | 413.24 |
| 28 | 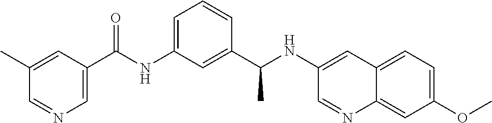 | 412.48 | 413.14 |
| 29 | 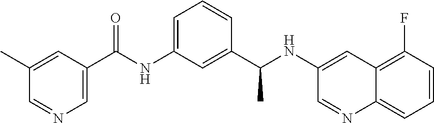 [SMILES: Cc1cncc(C(=O)Nc2cccc([C@H](C)Nc3cnc4cccc(F)c4c3)c2)c1] | 400.44 | 401.13 |
| 30 | 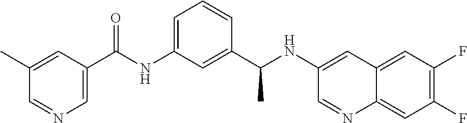 [SMILES: Cc1cncc(C(=O)Nc2cccc([C@H](C)Nc3cnc4cc(F)c(F)cc4c3)c2)c1] | 418.44 | 419.12 |
| 31 | 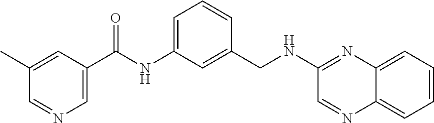 [SMILES: Cc1cncc(C(=O)Nc2cccc(CNc3cnc4ccccc4n3)c2)c1] | 369.42 | 370.18 |
| 32 | 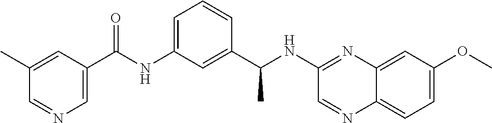 | 413.47 | 414.21 |
| 33 | 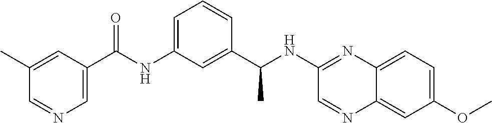 | 413.47 | 414.25 |
| 34 | 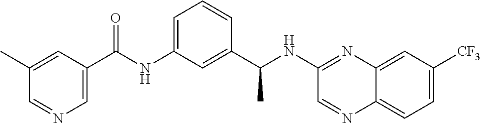 [SMILES: Cc1cncc(C(=O)Nc2cccc([C@H](C)Nc3cnc4ccc(C(F)(F)F)cc4n3)c2)c1] | 451.44 | 452.23 |
| 35 | 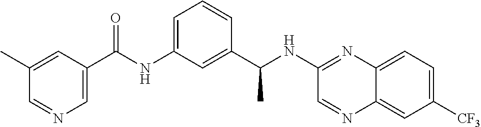 [SMILES: Cc1cncc(C(=O)Nc2cccc([C@H](C)Nc3cnc4cc(C(F)(F)F)ccc4n3)c2)c1] | 451.44 | 453.03 |
| 36 | 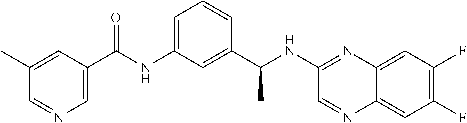 [SMILES: Cc1cncc(C(=O)Nc2cccc([C@H](C)Nc3cnc4cc(F)c(F)cc4n3)c2)c1] | 419.43 | 420.22 |
| 37 | 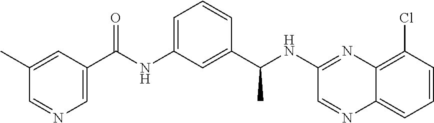 [SMILES: Cc1cncc(C(=O)Nc2cccc([C@H](C)Nc3cnc4cccc(Cl)c4n3)c2)c1] | 417.89 | 420.04 |
| 38 | 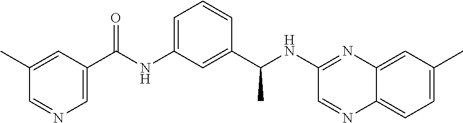 | 397.47 | 398.15 |
| 39 | 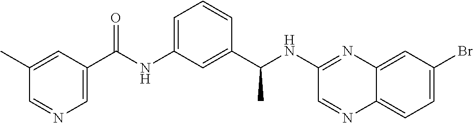 [SMILES: Cc1cncc(C(=O)Nc2cccc([C@H](C)Nc3cnc4ccc(Br)cc4n3)c2)c1] | 462.34 | 464.05 |
| 40 | 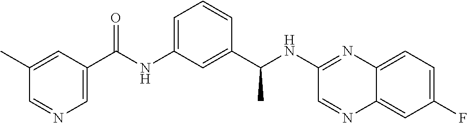 [SMILES: Cc1cncc(C(=O)Nc2cccc([C@H](C)Nc3cnc4cc(F)ccc4n3)c2)c1] | 401.44 | 402.41 |
| 41 | 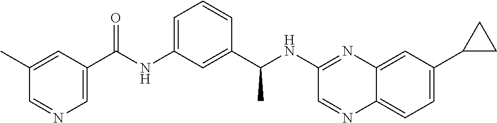 [SMILES: Cc1cncc(C(=O)Nc2cccc([C@H](C)Nc3cnc4ccc(C5CC5)cc4n3)c2)c1] | 423.51 | 424.24 |
| 42 | 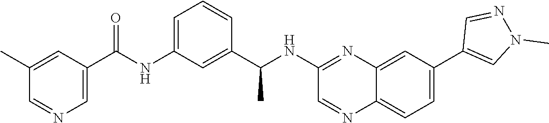 | 463.53 | 464.25 |
| 43 | 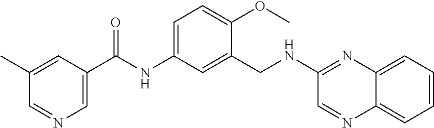 [SMILES: COc1ccc(NC(=O)c2cncc(C)c2)cc1CNc1cnc2ccccc2n1] | 399.44 | 400.11 |
| 44 | 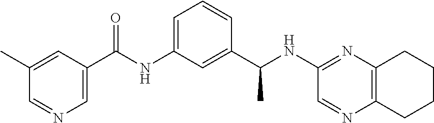 [SMILES: Cc1cncc(C(=O)Nc2cccc([C@H](C)Nc3cnc4c(n3)CCCC4)c2)c1] | 387.48 | 388.27 |
| 45 | 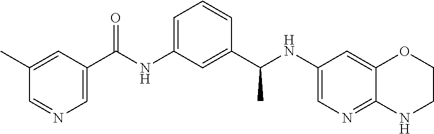 [SMILES: Cc1cncc(C(=O)Nc2cccc([C@H](C)Nc3cnc4c(c3)OCCN4)c2)c1] | 389.45 | 390.14 |
| 46 | 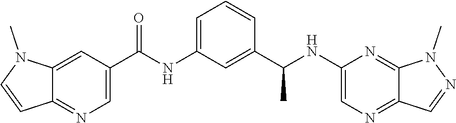 [SMILES: C[C@H](Nc1cnc2cnn(C)c2n1)c1cccc(NC(=O)c2cnc3ccn(C)c3c2)c1] | 426.47 | 428.03 |
| 47 | 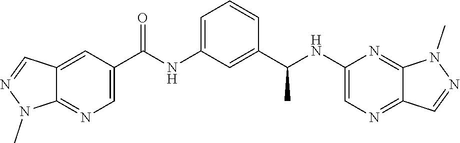 [SMILES: C[C@H](Nc1cnc2cnn(C)c2n1)c1cccc(NC(=O)c2cnc3c(cnn3C)c2)c1] | 427.46 | 427.80 |
| 48 | 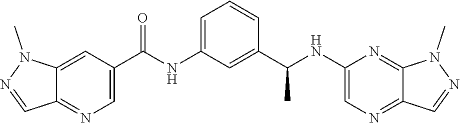 [SMILES: C[C@H](Nc1cnc2cnn(C)c2n1)c1cccc(NC(=O)c2cnc3cnn(C)c3c2)c1] | 427.46 | 428.80 |
| 49 | 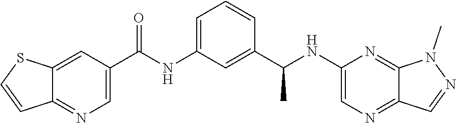 [SMILES: C[C@H](Nc1cnc2cnn(C)c2n1)c1cccc(NC(=O)c2cnc3ccsc3c2)c1] | 429.50 | 431.00 |
| 50 | 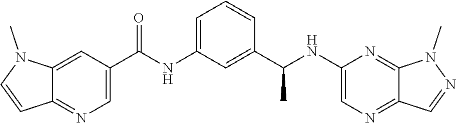 [SMILES: C[C@H](Nc1cnc2cnn(C)c2n1)c1cccc(NC(=O)c2cnc3ccn(C)c3c2)c1] | 425.49 | 426.20 |
| 51 | 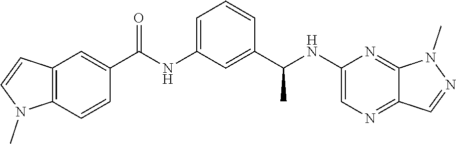 [SMILES: C[C@H](Nc1cnc2cnn(C)c2n1)c1cccc(NC(=O)c2ccc3c(ccn3C)c2)c1] | 425.49 | 426.30 |
| 52 | 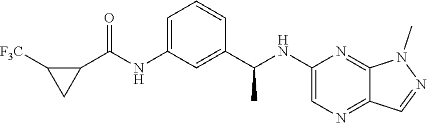 [SMILES: C[C@H](Nc1cnc2cnn(C)c2n1)c1cccc(NC(=O)C2CC2C(F)(F)F)c1] | 404.4 | 405.20 |
| 53 | 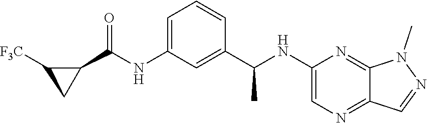 | 404.4 | 405.20 |
| 54 | 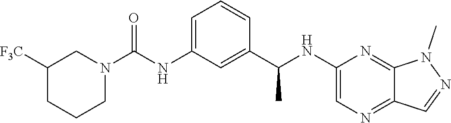 [SMILES: C[C@H](Nc1cnc2cnn(C)c2n1)c1cccc(NC(=O)N2CCCC(C(F)(F)F)C2)c1] | 447.47 | 448.30 |
| 55 | 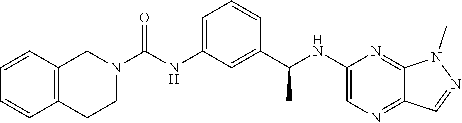 [SMILES: C[C@H](Nc1cnc2cnn(C)c2n1)c1cccc(NC(=O)N2CCc3ccccc3C2)c1] | 427.51 | 428.30 |
| 56 | 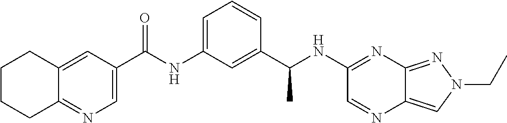 [SMILES: CCn1cc2ncc(N[C@@H](C)c3cccc(NC(=O)c4cnc5c(c4)CCCC5)c3)nc2n1] | 443.51 | 444.20 |
| 57 | 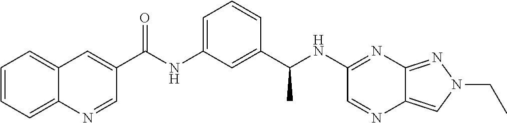 [SMILES: CCn1cc2ncc(N[C@@H](C)c3cccc(NC(=O)c4cnc5ccccc5c4)c3)nc2n1] | 437.5 | 438.2 |
| 58 | 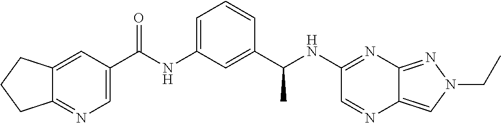 [SMILES: CCn1cc2ncc(N[C@@H](C)c3cccc(NC(=O)c4cnc5c(c4)CCC5)c3)nc2n1] | 427.5 | 428.3 |
| 59 | 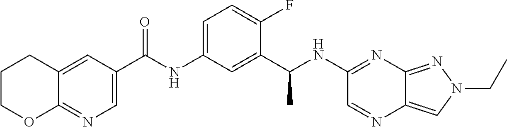 [SMILES: CCn1cc2ncc(N[C@@H](C)c3cc(NC(=O)c4cnc5c(c4)CCCO5)ccc3F)nc2n1] | 461.49 | 462.2 |
| 60 | 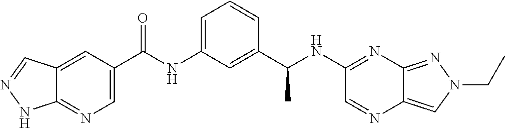 [SMILES: CCn1cc2ncc(N[C@@H](C)c3cccc(NC(=O)c4cnc5[nH]ncc5c4)c3)nc2n1] | 427.46 | 428.2 |
| 61 | 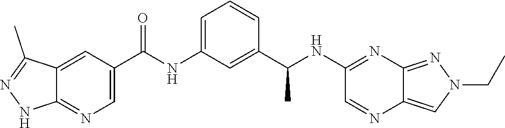 [SMILES: CCn1cc2ncc(N[C@@H](C)c3cccc(NC(=O)c4cnc5[nH]nc(C)c5c4)c3)nc2n1] | 441.49 | 442.2 |
| 62 | 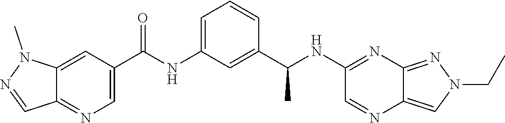 [SMILES: CCn1cc2ncc(N[C@@H](C)c3cccc(NC(=O)c4cnc5cnn(C)c5c4)c3)nc2n1] | 441.49 | 442.3 |
| 63 | 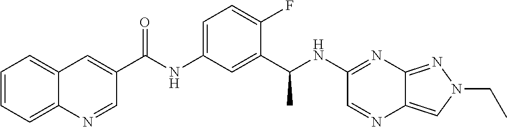 [SMILES: CCn1cc2ncc(N[C@@H](C)c3cc(NC(=O)c4cnc5ccccc5c4)ccc3F)nc2n1] | 455.49 | 456.2 |
| 64 | 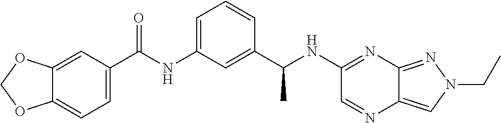 [SMILES: CCn1cc2ncc(N[C@@H](C)c3cccc(NC(=O)c4ccc5c(c4)OCO5)c3)nc2n1] | 430.46 | 432.4 |
| 65 | 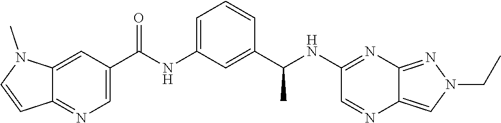 [SMILES: CCn1cc2ncc(N[C@@H](C)c3cccc(NC(=O)c4cnc5ccn(C)c5c4)c3)nc2n1] | 440.5 | 441.3 |
| 66 | 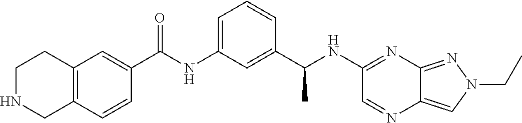 [SMILES: CCn1cc2ncc(N[C@@H](C)c3cccc(NC(=O)c4ccc5c(c4)CCNC5)c3)nc2n1] | 441.53 | 442.5 |
| 67 | 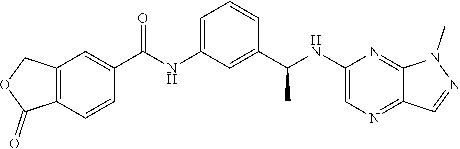 [SMILES: C[C@H](Nc1cnc2cnn(C)c2n1)c1cccc(NC(=O)c2ccc3c(c2)COC3=O)c1] | 428.44 | 429.1 |
| 68 | 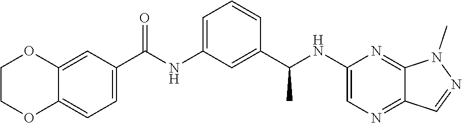 [SMILES: C[C@H](Nc1cnc2cnn(C)c2n1)c1cccc(NC(=O)c2ccc3c(c2)OCCO3)c1] | 430.46 | 431.1 |
| 69 | 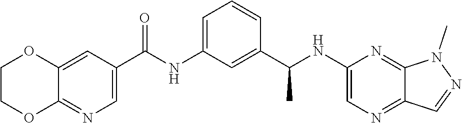 [SMILES: C[C@H](Nc1cnc2cnn(C)c2n1)c1cccc(NC(=O)c2cnc3c(c2)OCCO3)c1] | 431.45 | 432.3 |
| 70 | 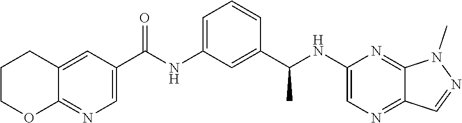 [SMILES: C[C@H](Nc1cnc2cnn(C)c2n1)c1cccc(NC(=O)c2cnc3c(c2)CCCO3)c1] | 429.47 | 430.2 |
| 71 | 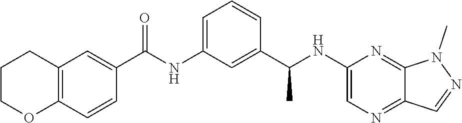 [SMILES: C[C@H](Nc1cnc2cnn(C)c2n1)c1cccc(NC(=O)c2ccc3c(c2)CCCO3)c1] | 428.49 | 429.1 |
| 72 | 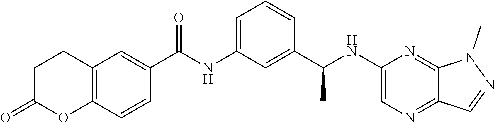 [SMILES: C[C@H](Nc1cnc2cnn(C)c2n1)c1cccc(NC(=O)c2ccc3c(c2)CCC(=O)O3)c1] | 442.47 | 443.3 |
| 73 | 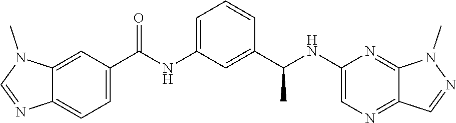 [SMILES: C[C@H](Nc1cnc2cnn(C)c2n1)c1cccc(NC(=O)c2ccc3ncn(C)c3c2)c1] | 426.47 | 427.2 |
| 74 | 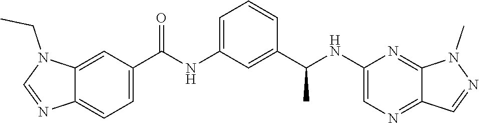 [SMILES: CCn1cnc2ccc(C(=O)Nc3cccc([C@H](C)Nc4cnc5cnn(C)c5n4)c3)cc21] | 440.5 | 441.9 |
| 75 | 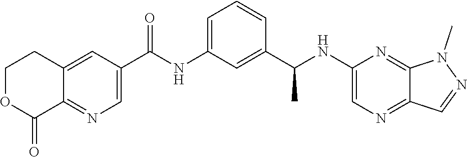 [SMILES: C[C@H](Nc1cnc2cnn(C)c2n1)c1cccc(NC(=O)c2cnc3c(c2)CCOC3=O)c1] | 443.46 | 443.2 |
| 76 | 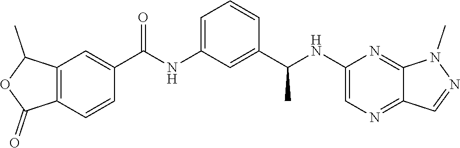 [SMILES: CC1OC(=O)c2ccc(C(=O)Nc3cccc([C@H](C)Nc4cnc5cnn(C)c5n4)c3)cc21] | 442.47 | 443.3 |
| 77 | 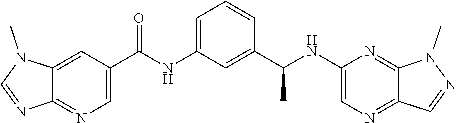 [SMILES: C[C@H](Nc1cnc2cnn(C)c2n1)c1cccc(NC(=O)c2cnc3ncn(C)c3c2)c1] | 427.46 | 428.1 |
| 78 | 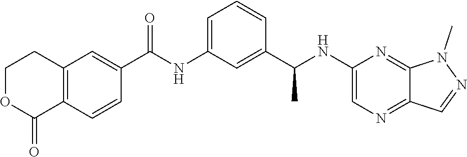 [SMILES: C[C@H](Nc1cnc2cnn(C)c2n1)c1cccc(NC(=O)c2ccc3c(c2)CCOC3=O)c1] | 442.47 | 444.1 |
| 79 | 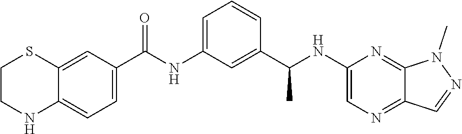 [SMILES: C[C@H](Nc1cnc2cnn(C)c2n1)c1cccc(NC(=O)c2ccc3c(c2)SCCN3)c1] | 445.54 | 446.1 |
| 80 | 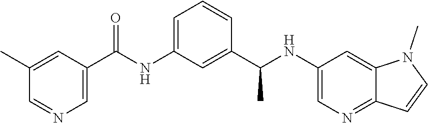 [SMILES: Cc1cncc(C(=O)Nc2cccc([C@H](C)Nc3cnc4ccn(C)c4c3)c2)c1] | 385.46 | 386.14 |
| 81 | 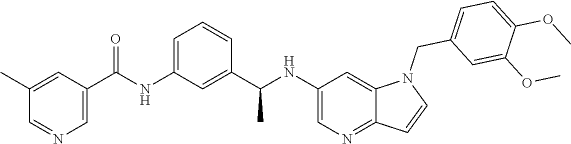 | 521.61 | 522.10 |
| 82 | 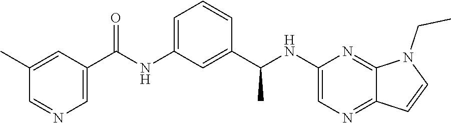 [SMILES: CCn1ccc2ncc(N[C@@H](C)c3cccc(NC(=O)c4cncc(C)c4)c3)nc21] | 400.48 | 401.30 |
| 83 | 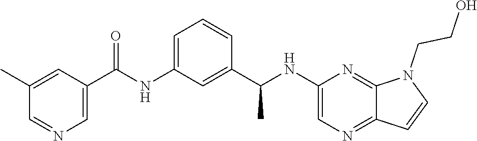 [SMILES: Cc1cncc(C(=O)Nc2cccc([C@H](C)Nc3cnc4ccn(CCO)c4n3)c2)c1] | 416.47 | 417.25 |
| 84 | 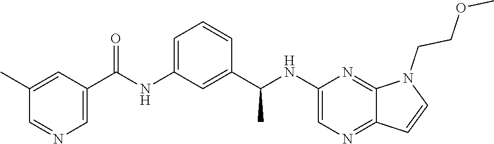 | 430.5 | 431.25 |
| 85 | 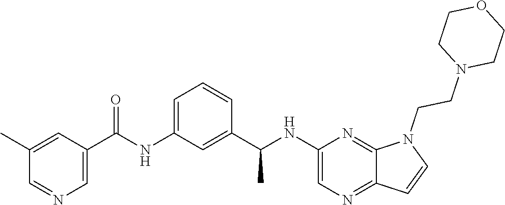 [SMILES: Cc1cncc(C(=O)Nc2cccc([C@H](C)Nc3cnc4ccn(CCN5CCOCC5)c4n3)c2)c1] | 485.58 | 486.35 |
| 86 | 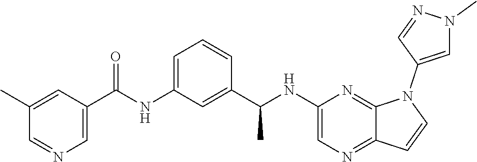 | 452.51 | 453.30 |
| 87 | 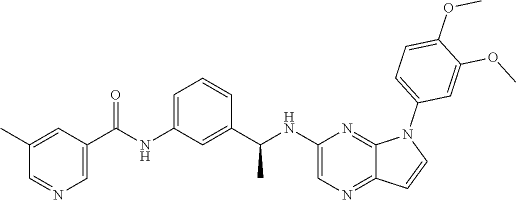 | 508.57 | 509.10 |
| 88 | 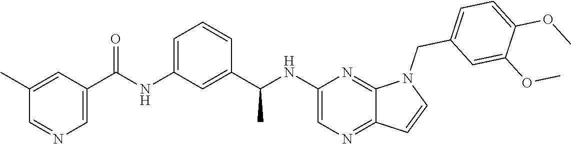 | 522.6 | 523.10 |
| 89 | 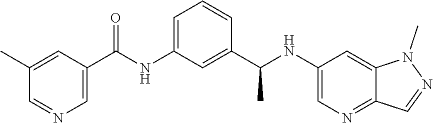 [SMILES: Cc1cncc(C(=O)Nc2cccc([C@H](C)Nc3cnc4cnn(C)c4c3)c2)c1] | 386.45 | 387.13 |
| 90 | 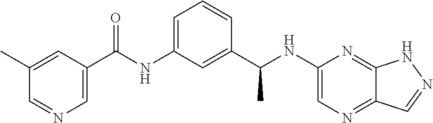 [SMILES: Cc1cncc(C(=O)Nc2cccc([C@H](C)Nc3cnc4cn[nH]c4n3)c2)c1] | 373.41 | 374.30 |
| 91 | 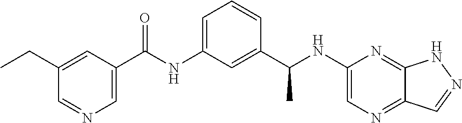 [SMILES: CCc1cncc(C(=O)Nc2cccc([C@H](C)Nc3cnc4cn[nH]c4n3)c2)c1] | 387.45 | 388.00 |
| 92 | 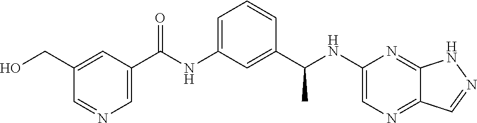 [SMILES: C[C@H](Nc1cnc2cn[nH]c2n1)c1cccc(NC(=O)c2cncc(CO)c2)c1] | 389.42 | 390.00 |
| 93 | 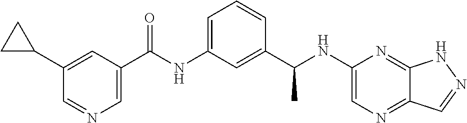 [SMILES: C[C@H](Nc1cnc2cn[nH]c2n1)c1cccc(NC(=O)c2cncc(C3CC3)c2)c1] | 399.46 | 400.20 |
| 94 | 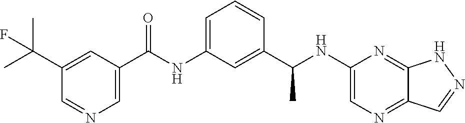 [SMILES: C[C@H](Nc1cnc2cn[nH]c2n1)c1cccc(NC(=O)c2cncc(C(C)(C)F)c2)c1] | 419.46 | 420.25 |
| 95 | 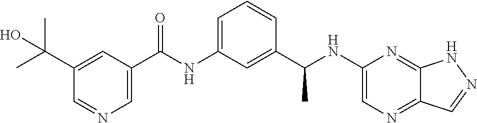 [SMILES: C[C@H](Nc1cnc2cn[nH]c2n1)c1cccc(NC(=O)c2cncc(C(C)(C)O)c2)c1] | 417.47 | 418.25 |
| 96 | 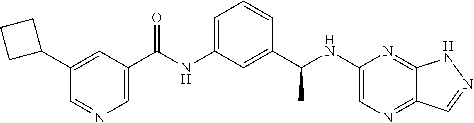 [SMILES: C[C@H](Nc1cnc2cn[nH]c2n1)c1cccc(NC(=O)c2cncc(C3CCC3)c2)c1] | 413.49 | 414.30 |
| 97 | 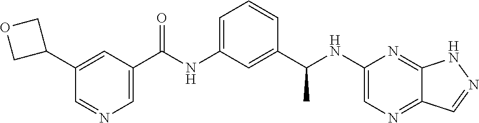 [SMILES: C[C@H](Nc1cnc2cn[nH]c2n1)c1cccc(NC(=O)c2cncc(C3COC3)c2)c1] | 415.46 | 416.25 |
| 98 | 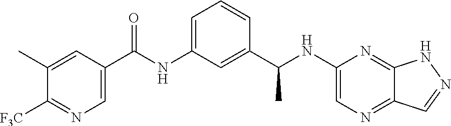 [SMILES: Cc1cc(C(=O)Nc2cccc([C@H](C)Nc3cnc4cn[nH]c4n3)c2)cnc1C(F)(F)F] | 441.42 | 442.20 |
| 99 | 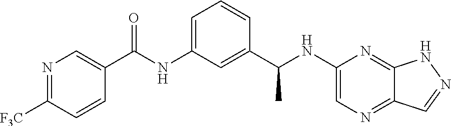 [SMILES: C[C@H](Nc1cnc2cn[nH]c2n1)c1cccc(NC(=O)c2ccc(C(F)(F)F)nc2)c1] | 427.39 | 428.20 |
| 100 | 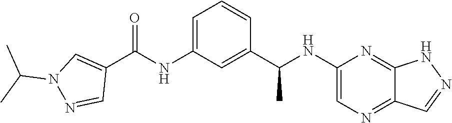 [SMILES: CC(C)n1cc(C(=O)Nc2cccc([C@H](C)Nc3cnc4cn[nH]c4n3)c2)cn1] | 390.45 | 391.20 |
| 101 | 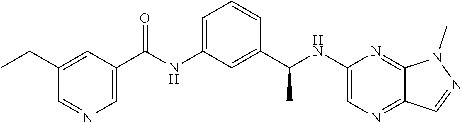 [SMILES: CCc1cncc(C(=O)Nc2cccc([C@H](C)Nc3cnc4cnn(C)c4n3)c2)c1] | 401.47 | 402.25 |
| 102 | 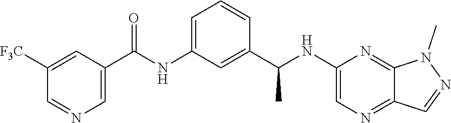 [SMILES: C[C@H](Nc1cnc2cnn(C)c2n1)c1cccc(NC(=O)c2cncc(C(F)(F)F)c2)c1] | 441.41 | 442.15 |
| 103 | 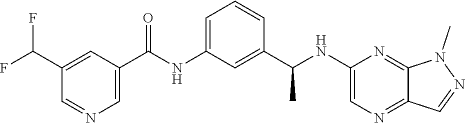 [SMILES: C[C@H](Nc1cnc2cnn(C)c2n1)c1cccc(NC(=O)c2cncc(C(F)F)c2)c1] | 423.42 | 424.71 |
| 104 | 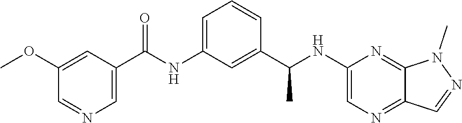 [SMILES: COc1cncc(C(=O)Nc2cccc([C@H](C)Nc3cnc4cnn(C)c4n3)c2)c1] | 403.44 | 404.73 |
| 105 | 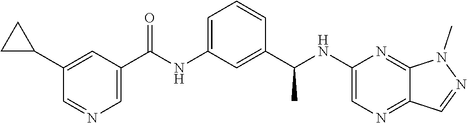 [SMILES: C[C@H](Nc1cnc2cnn(C)c2n1)c1cccc(NC(=O)c2cncc(C3CC3)c2)c1] | 413.49 | 414.25 |
| 106 | 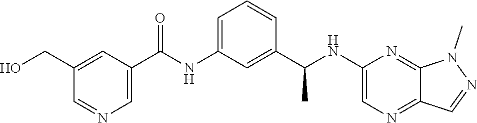 [SMILES: C[C@H](Nc1cnc2cnn(C)c2n1)c1cccc(NC(=O)c2cncc(CO)c2)c1] | 403.45 | 404.25 |
| 107 | 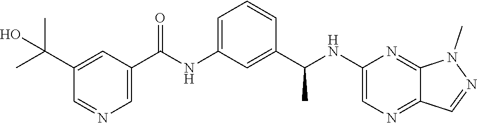 [SMILES: C[C@H](Nc1cnc2cnn(C)c2n1)c1cccc(NC(=O)c2cncc(C(C)(C)O)c2)c1] | 431.5 | 432.30 |
| 108 | 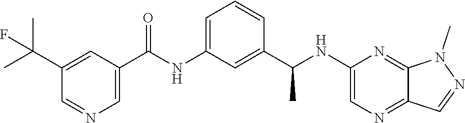 [SMILES: C[C@H](Nc1cnc2cnn(C)c2n1)c1cccc(NC(=O)c2cncc(C(C)(C)F)c2)c1] | 433.49 | 434.25 |
| 109 | 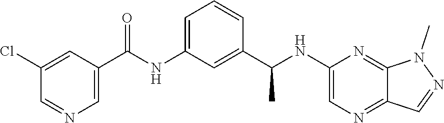 [SMILES: C[C@H](Nc1cnc2cnn(C)c2n1)c1cccc(NC(=O)c2cncc(Cl)c2)c1] | 407.86 | 408.22 |
| 110 | 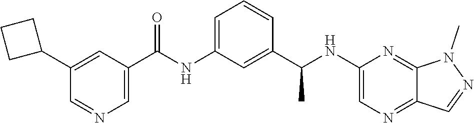 [SMILES: C[C@H](Nc1cnc2cnn(C)c2n1)c1cccc(NC(=O)c2cncc(C3CCC3)c2)c1] | 427.51 | 428.00 |
| 111 | 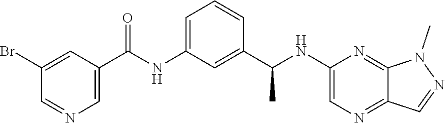 [SMILES: C[C@H](Nc1cnc2cnn(C)c2n1)c1cccc(NC(=O)c2cncc(Br)c2)c1] | 452.31 | 454.10 |
| 112 | 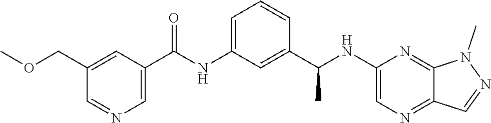 [SMILES: COCc1cncc(C(=O)Nc2cccc([C@H](C)Nc3cnc4cnn(C)c4n3)c2)c1] | 417.46 | 419.00 |
| 113 | 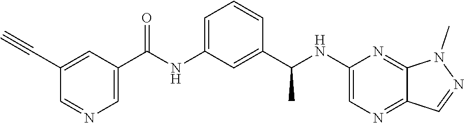 [SMILES: C#Cc1cncc(C(=O)Nc2cccc([C@H](C)Nc3cnc4cnn(C)c4n3)c2)c1] | 397.43 | 398.90 |
| 114 | 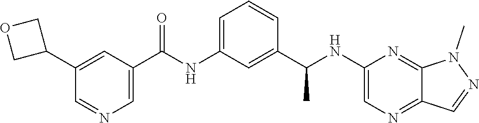 [SMILES: C[C@H](Nc1cnc2cnn(C)c2n1)c1cccc(NC(=O)c2cncc(C3COC3)c2)c1] | 429.48 | 430.25 |
| 115 | 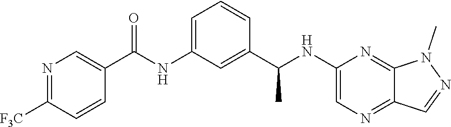 [SMILES: C[C@H](Nc1cnc2cnn(C)c2n1)c1cccc(NC(=O)c2ccc(C(F)(F)F)nc2)c1] | 441.41 | 442.15 |
| 116 | 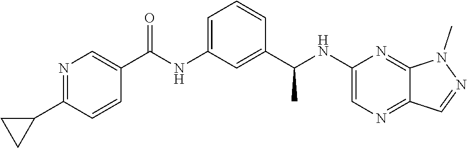 [SMILES: C[C@H](Nc1cnc2cnn(C)c2n1)c1cccc(NC(=O)c2ccc(C3CC3)nc2)c1] | 413.49 | 414.25 |
| 117 | 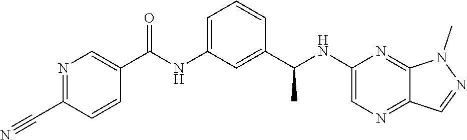 [SMILES: C[C@H](Nc1cnc2cnn(C)c2n1)c1cccc(NC(=O)c2ccc(C#N)nc2)c1] | 398.43 | 399.00 |
| 118 | 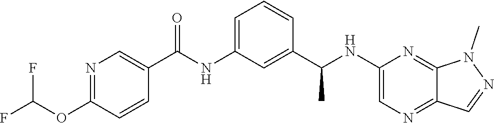 [SMILES: C[C@H](Nc1cnc2cnn(C)c2n1)c1cccc(NC(=O)c2ccc(OC(F)F)nc2)c1] | 439.43 | 440.20 |
| 119 | 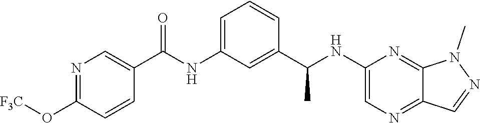 [SMILES: C[C@H](Nc1cnc2cnn(C)c2n1)c1cccc(NC(=O)c2ccc(OC(F)(F)F)nc2)c1] | 457.42 | 458.20 |
| 120 | 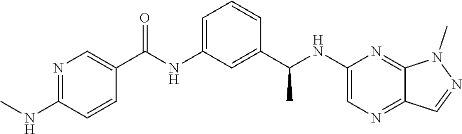 [SMILES: CNc1ccc(C(=O)Nc2cccc([C@H](C)Nc3cnc4cnn(C)c4n3)c2)cn1] | 402.46 | 403.10 |
| 121 | 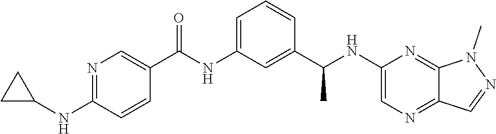 [SMILES: C[C@H](Nc1cnc2cnn(C)c2n1)c1cccc(NC(=O)c2ccc(NC3CC3)nc2)c1] | 428.5 | 429.00 |
| 122 | 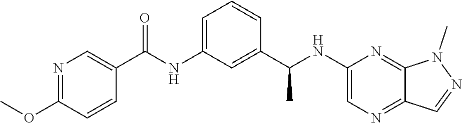 [SMILES: COc1ccc(C(=O)Nc2cccc([C@H](C)Nc3cnc4cnn(C)c4n3)c2)cn1] | 403.45 | 404.00 |
| 123 | 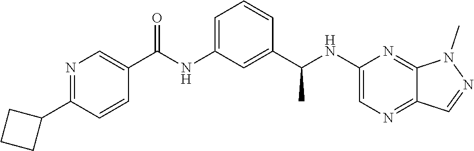 [SMILES: C[C@H](Nc1cnc2cnn(C)c2n1)c1cccc(NC(=O)c2ccc(C3CCC3)nc2)c1] | 427.51 | 428.30 |
| 124 | 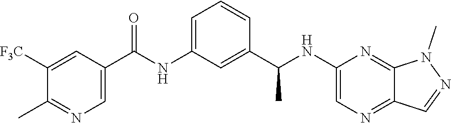 [SMILES: Cc1ncc(C(=O)Nc2cccc([C@H](C)Nc3cnc4cnn(C)c4n3)c2)cc1C(F)(F)F] | 455.44 | 456.20 |
| 125 | 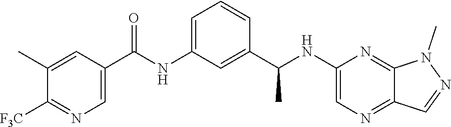 [SMILES: Cc1cc(C(=O)Nc2cccc([C@H](C)Nc3cnc4cnn(C)c4n3)c2)cnc1C(F)(F)F] | 455.44 | 456.15 |
| 126 | 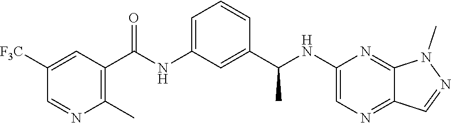 [SMILES: Cc1ncc(C(F)(F)F)cc1C(=O)Nc1cccc([C@H](C)Nc2cnc3cnn(C)c3n2)c1] | 455.44 | 456.15 |
| 127 | 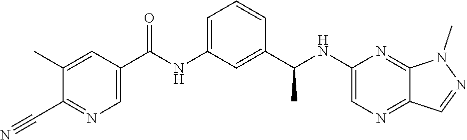 [SMILES: Cc1cc(C(=O)Nc2cccc([C@H](C)Nc3cnc4cnn(C)c4n3)c2)cnc1C#N] | 412.46 | 413.15 |
| 128 | 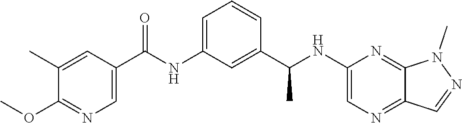 [SMILES: COc1ncc(C(=O)Nc2cccc([C@H](C)Nc3cnc4cnn(C)c4n3)c2)cc1C] | 417.47 | 418.25 |
| 129 | 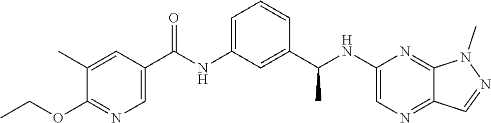 [SMILES: CCOc1ncc(C(=O)Nc2cccc([C@H](C)Nc3cnc4cnn(C)c4n3)c2)cc1C] | 431.5 | 432.20 |
| 130 | 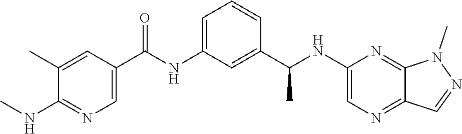 [SMILES: CNc1ncc(C(=O)Nc2cccc([C@H](C)Nc3cnc4cnn(C)c4n3)c2)cc1C] | 416.49 | 417.25 |
| 131 | 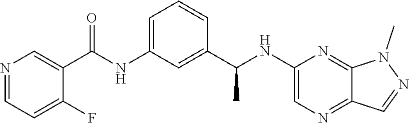 [SMILES: C[C@H](Nc1cnc2cnn(C)c2n1)c1cccc(NC(=O)c2cnccc2F)c1] | 391.41 | 392.20 |
| 132 | 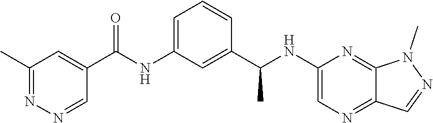 [SMILES: Cc1cc(C(=O)Nc2cccc([C@H](C)Nc3cnc4cnn(C)c4n3)c2)cnn1] | 388.43 | 389.30 |
| 133 | 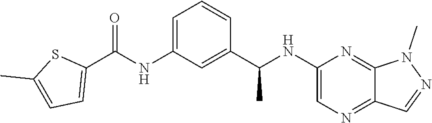 | 392.48 | 393.15 |
| 134 | 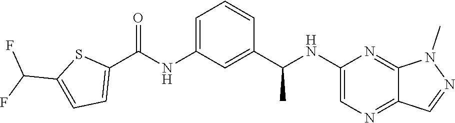 | 428.46 | 429.20 |
| 135 | 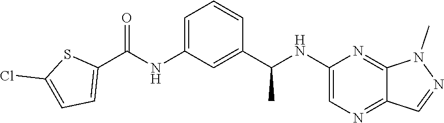 | 412.9 | 412.90 |
| 136 | 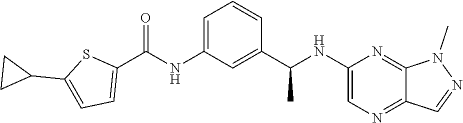 | 418.52 | 419.15 |
| 137 | 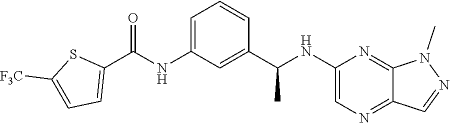 | 446.45 | 447.15 |
| 138 | 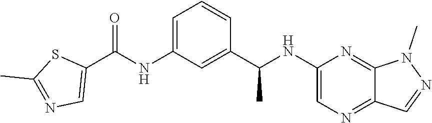 | 393.47 | 394.00 |
| 139 | 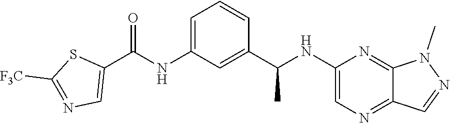 | 447.44 | 448.15 |
| 140 | 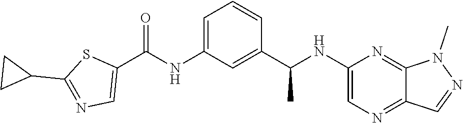 | 419.51 | 420.25 |
| 141 | 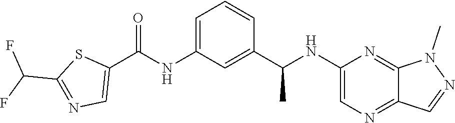 | 429.45 | 430.00 |
| 142 | 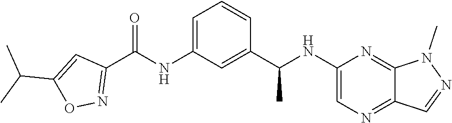 | 405.45 | 406.15 |
| 143 | 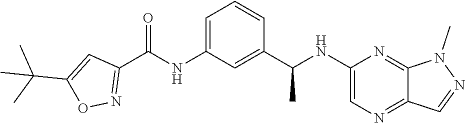 | 419.48 | 420.25 |
| 144 | 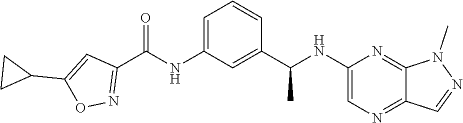 [SMILES: C[C@H](Nc1cnc2cnn(C)c2n1)c1cccc(NC(=O)c2cc(C3CC3)on2)c1] | 403.44 | 404.20 |
| 145 | 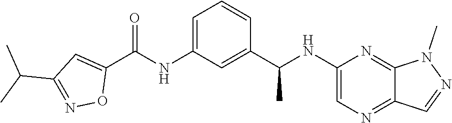 [SMILES: CC(C)c1cc(C(=O)Nc2cccc([C@H](C)Nc3cnc4cnn(C)c4n3)c2)on1] | 405.45 | 406.15 |
| 146 | 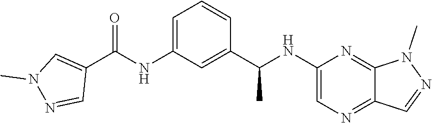 [SMILES: C[C@H](Nc1cnc2cnn(C)c2n1)c1cccc(NC(=O)c2cnn(C)c2)c1] | 376.41 | 377.20 |
| 147 | 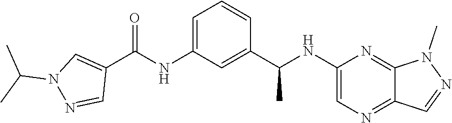 [SMILES: CC(C)n1cc(C(=O)Nc2cccc([C@H](C)Nc3cnc4cnn(C)c4n3)c2)cn1] | 404.47 | 405.20 |
| 148 | 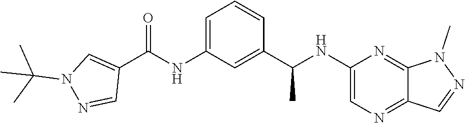 [SMILES: C[C@H](Nc1cnc2cnn(C)c2n1)c1cccc(NC(=O)c2cnn(C(C)(C)C)c2)c1] | 418.49 | 419.40 |
| 149 | 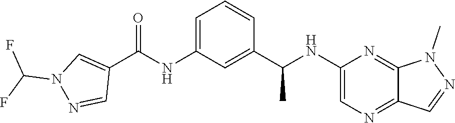 [SMILES: C[C@H](Nc1cnc2cnn(C)c2n1)c1cccc(NC(=O)c2cnn(C(F)F)c2)c1] | 412.40 | 413.94 |
| 150 | 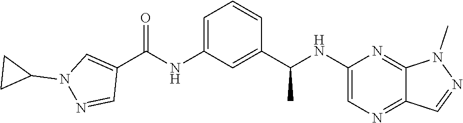 [SMILES: C[C@H](Nc1cnc2cnn(C)c2n1)c1cccc(NC(=O)c2cnn(C3CC3)c2)c1] | 402.45 | 403.86 |
| 151 | 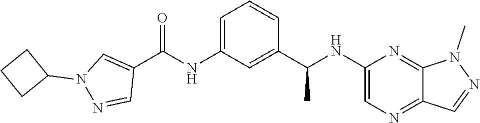 [SMILES: C[C@H](Nc1cnc2cnn(C)c2n1)c1cccc(NC(=O)c2cnn(C3CCC3)c2)c1] | 416.48 | 417.82 |
| 152 | 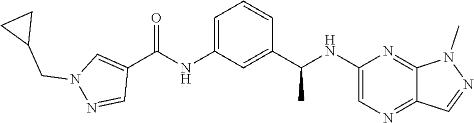 [SMILES: C[C@H](Nc1cnc2cnn(C)c2n1)c1cccc(NC(=O)c2cnn(CC3CC3)c2)c1] | 416.48 | 417.97 |
| 153 | 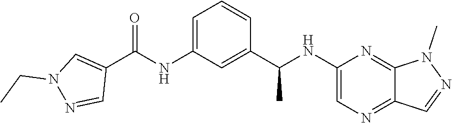 [SMILES: CCn1cc(C(=O)Nc2cccc([C@H](C)Nc3cnc4cnn(C)c4n3)c2)cn1] | 390.44 | 391.66 |
| 154 | 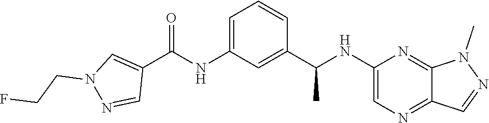 [SMILES: C[C@H](Nc1cnc2cnn(C)c2n1)c1cccc(NC(=O)c2cnn(CCF)c2)c1] | 408.43 | 409.70 |
| 155 | 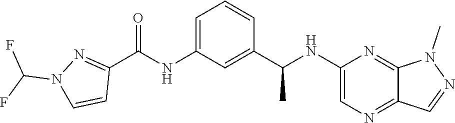 [SMILES: C[C@H](Nc1cnc2cnn(C)c2n1)c1cccc(NC(=O)c2ccn(C(F)F)n2)c1] | 412.40 | 413.98 |
| 156 | 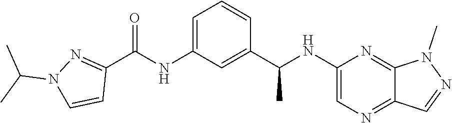 [SMILES: CC(C)n1ccc(C(=O)Nc2cccc([C@H](C)Nc3cnc4cnn(C)c4n3)c2)n1] | 404.47 | 405.62 |
| 157 | 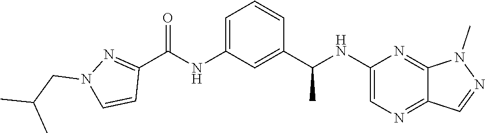 [SMILES: CC(C)Cn1ccc(C(=O)Nc2cccc([C@H](C)Nc3cnc4cnn(C)c4n3)c2)n1] | 418.49 | 419.50 |
| 158 | 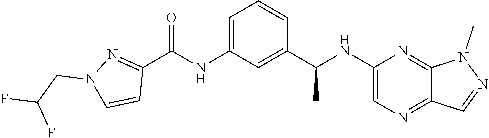 [SMILES: C[C@H](Nc1cnc2cnn(C)c2n1)c1cccc(NC(=O)c2ccn(CC(F)F)n2)c1] | 426.42 | 428.00 |
| 159 | 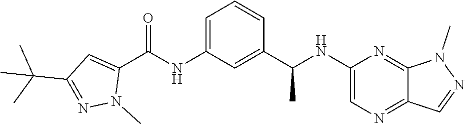 [SMILES: C[C@H](Nc1cnc2cnn(C)c2n1)c1cccc(NC(=O)c2cc(C(C)(C)C)nn2C)c1] | 432.52 | 433.25 |
| 160 | 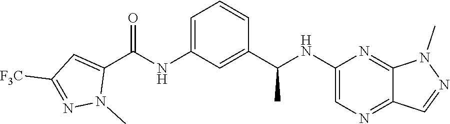 [SMILES: C[C@H](Nc1cnc2cnn(C)c2n1)c1cccc(NC(=O)c2cc(C(F)(F)F)nn2C)c1] | 444.41 | 445.15 |
| 161 | 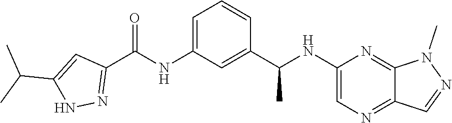 [SMILES: CC(C)c1cc(C(=O)Nc2cccc([C@H](C)Nc3cnc4cnn(C)c4n3)c2)n[nH]1] | 404.47 | 405.20 |
| 162 | 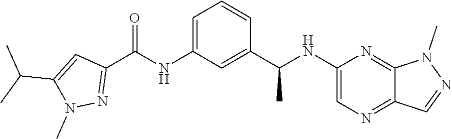 [SMILES: CC(C)c1cc(C(=O)Nc2cccc([C@H](C)Nc3cnc4cnn(C)c4n3)c2)nn1C] | 418.49 | 419.25 |
| 163 | 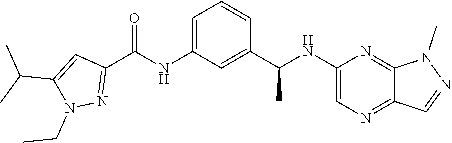 [SMILES: CCn1nc(C(=O)Nc2cccc([C@H](C)Nc3cnc4cnn(C)c4n3)c2)cc1C(C)C] | 432.52 | 433.25 |
| 164 | 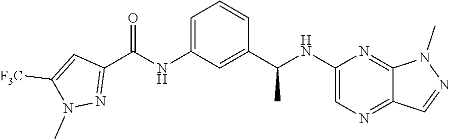 [SMILES: C[C@H](Nc1cnc2cnn(C)c2n1)c1cccc(NC(=O)c2cc(C(F)(F)F)n(C)n2)c1] | 444.41 | 445.20 |
| 165 | 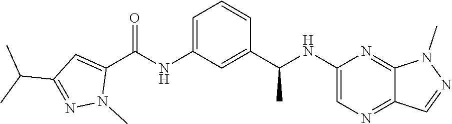 [SMILES: CC(C)c1cc(C(=O)Nc2cccc([C@H](C)Nc3cnc4cnn(C)c4n3)c2)n(C)n1] | 418.49 | 419.30 |
| 166 | 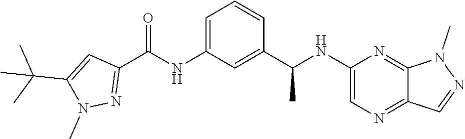 [SMILES: C[C@H](Nc1cnc2cnn(C)c2n1)c1cccc(NC(=O)c2cc(C(C)(C)C)n(C)n2)c1] | 432.52 | 433.25 |
| 167 | 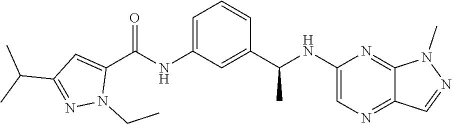 [SMILES: CCn1nc(C(C)C)cc1C(=O)Nc1cccc([C@H](C)Nc2cnc3cnn(C)c3n2)c1] | 432.52 | 433.30 |
| 168 | 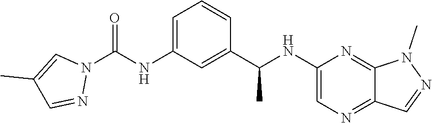 [SMILES: Cc1cnn(C(=O)Nc2cccc([C@H](C)Nc3cnc4cnn(C)c4n3)c2)c1] | 376.42 | 377.25 |
| 169 | 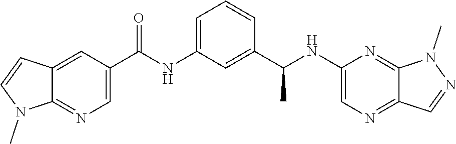 [SMILES: C[C@H](Nc1cnc2cnn(C)c2n1)c1cccc(NC(=O)c2cnc3c(ccn3C)c2)c1] | 426.47 | 427.20 |
| 170 | 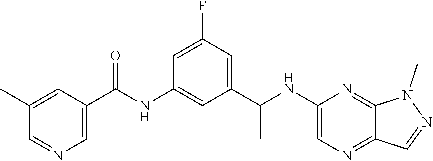 [SMILES: Cc1cncc(C(=O)Nc2cc(F)cc(C(C)Nc3cnc4cnn(C)c4n3)c2)c1] | 405.44 | 406.20 |
| 171 | 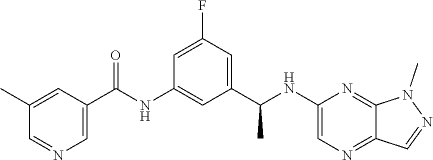 [SMILES: Cc1cncc(C(=O)Nc2cc(F)cc([C@H](C)Nc3cnc4cnn(C)c4n3)c2)c1] | 405.44 | 406.15 |
| 172 | 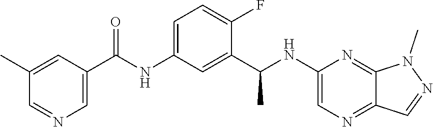 [SMILES: Cc1cncc(C(=O)Nc2ccc(F)c([C@H](C)Nc3cnc4cnn(C)c4n3)c2)c1] | 405.44 | 406.00 |
| 173 | 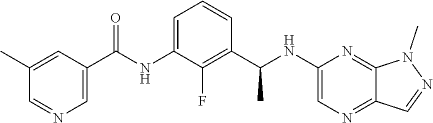 [SMILES: Cc1cncc(C(=O)Nc2cccc([C@H](C)Nc3cnc4cnn(C)c4n3)c2F)c1] | 405.44 | 406.10 |
| 174 | 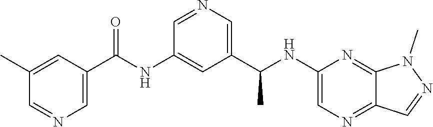 [SMILES: Cc1cncc(C(=O)Nc2cncc([C@H](C)Nc3cnc4cnn(C)c4n3)c2)c1] | 388.44 | 389.10 |
| 175 | 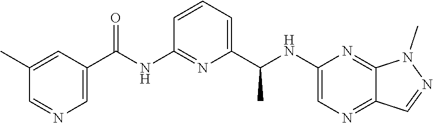 [SMILES: Cc1cncc(C(=O)Nc2cccc([C@H](C)Nc3cnc4cnn(C)c4n3)n2)c1] | 388.44 | 389.15 |
| 176 | 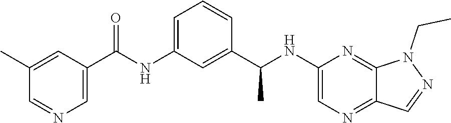 [SMILES: CCn1ncc2ncc(N[C@@H](C)c3cccc(NC(=O)c4cncc(C)c4)c3)nc21] | 401.47 | 402.25 |
| 177 | 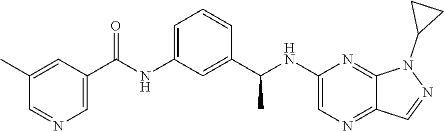 [SMILES: Cc1cncc(C(=O)Nc2cccc([C@H](C)Nc3cnc4cnn(C5CC5)c4n3)c2)c1] | 413.47 | 414.10 |
| 178 | 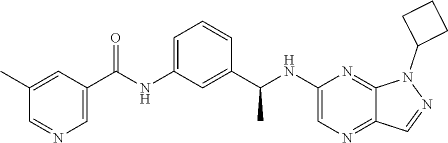 [SMILES: Cc1cncc(C(=O)Nc2cccc([C@H](C)Nc3cnc4cnn(C5CCC5)c4n3)c2)c1] | 427.51 | 428.25 |
| 179 | 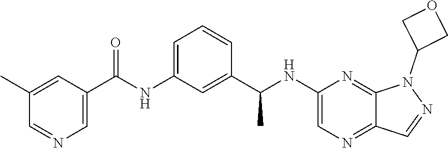 [SMILES: Cc1cncc(C(=O)Nc2cccc([C@H](C)Nc3cnc4cnn(C5COC5)c4n3)c2)c1] | 429.48 | 430.30 |
| 180 | 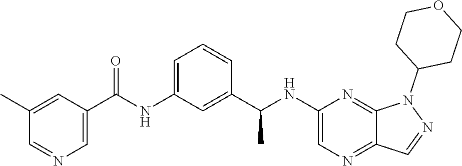 [SMILES: Cc1cncc(C(=O)Nc2cccc([C@H](C)Nc3cnc4cnn(C5CCOCC5)c4n3)c2)c1] | 457.53 | 458.29 |
| 181 | 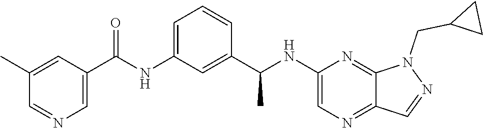 [SMILES: Cc1cncc(C(=O)Nc2cccc([C@H](C)Nc3cnc4cnn(CC5CC5)c4n3)c2)c1] | 427.51 | 428.30 |
| 182 | 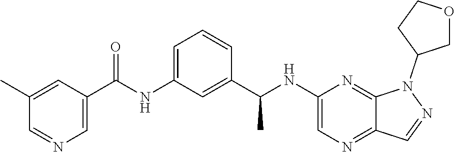 [SMILES: Cc1cncc(C(=O)Nc2cccc([C@H](C)Nc3cnc4cnn(C5CCOC5)c4n3)c2)c1] | 443.51 | 444.30 |
| 183 | 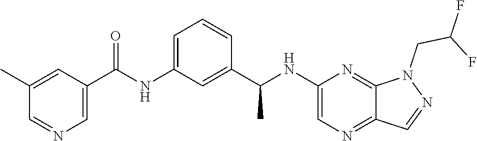 [SMILES: Cc1cncc(C(=O)Nc2cccc([C@H](C)Nc3cnc4cnn(CC(F)F)c4n3)c2)c1] | 437.45 | 438.25 |
| 184 | 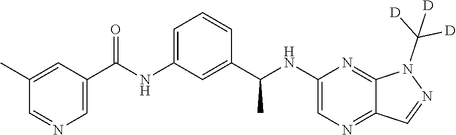 [SMILES: [2H]C([2H])([2H])n1ncc2ncc(N[C@@H](C)c3cccc(NC(=O)c4cncc(C)c4)c3)nc21] | 390.47 | 391.15 |
| 185 | 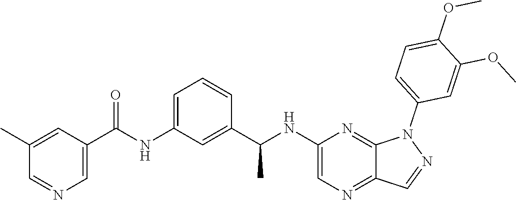 | 509.56 | 510.40 |
| 186 | 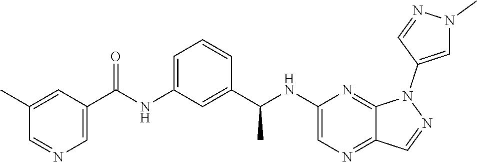 | 453.5 | 454.3 |
| 187 | 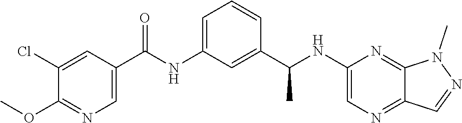 [SMILES: COc1ncc(C(=O)Nc2cccc([C@H](C)Nc3cnc4cnn(C)c4n3)c2)cc1Cl] | 437.88 | 438.5 |
| 188 | 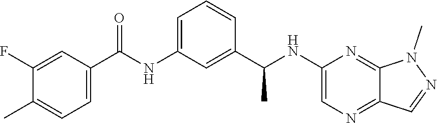 [SMILES: Cc1ccc(C(=O)Nc2cccc([C@H](C)Nc3cnc4cnn(C)c4n3)c2)cc1F] | 404.44 | 405.1 |
| 189 | 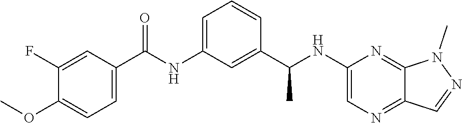 [SMILES: COc1ccc(C(=O)Nc2cccc([C@H](C)Nc3cnc4cnn(C)c4n3)c2)cc1F] | 420.44 | 421.5 |
| 190 | 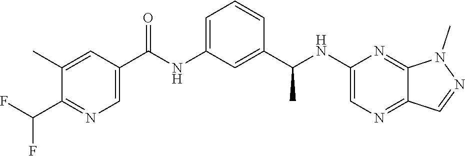 [SMILES: Cc1cc(C(=O)Nc2cccc([C@H](C)Nc3cnc4cnn(C)c4n3)c2)cnc1C(F)F] | 437.44 | 438.8 |
| 191 | 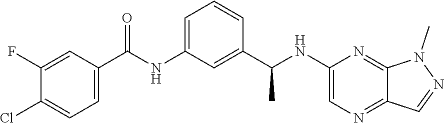 [SMILES: C[C@H](Nc1cnc2cnn(C)c2n1)c1cccc(NC(=O)c2ccc(Cl)c(F)c2)c1] | 424.86 | 425.5 |
| 192 | 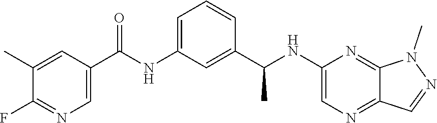 [SMILES: Cc1cc(C(=O)Nc2cccc([C@H](C)Nc3cnc4cnn(C)c4n3)c2)cnc1F] | 405.43 | 407.1 |
| 193 | 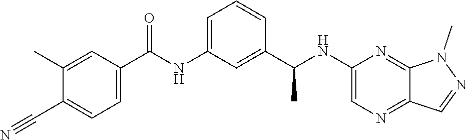 [SMILES: Cc1cc(C(=O)Nc2cccc([C@H](C)Nc3cnc4cnn(C)c4n3)c2)ccc1C#N] | 411.46 | 413.3 |
| 194 | 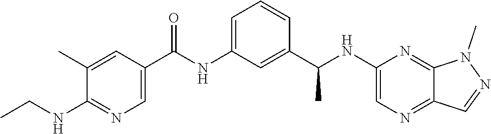 [SMILES: CCNc1ncc(C(=O)Nc2cccc([C@H](C)Nc3cnc4cnn(C)c4n3)c2)cc1C] | 430.5 | 432.1 |
| 195 | 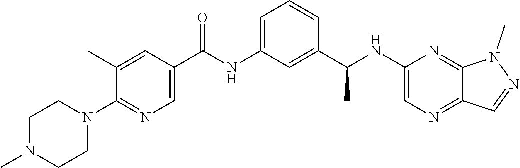 [SMILES: Cc1cc(C(=O)Nc2cccc([C@H](C)Nc3cnc4cnn(C)c4n3)c2)cnc1N1CCN(C)CC1] | 485.58 | 486.6 |
| 196 | 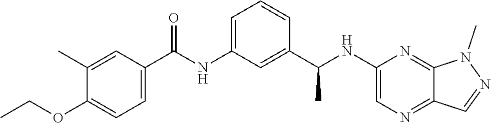 [SMILES: CCOc1ccc(C(=O)Nc2cccc([C@H](C)Nc3cnc4cnn(C)c4n3)c2)cc1C] | 430.5 | 431.5 |
| 197 | 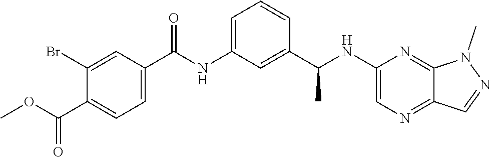 [SMILES: COC(=O)c1ccc(C(=O)Nc2cccc([C@H](C)Nc3cnc4cnn(C)c4n3)c2)cc1Br] | 509.35 | 511.3 |
| 198 | 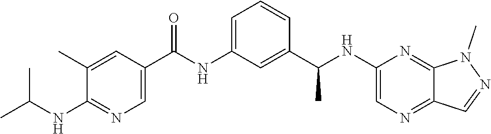 [SMILES: Cc1cc(C(=O)Nc2cccc([C@H](C)Nc3cnc4cnn(C)c4n3)c2)cnc1NC(C)C] | 444.53 | 446.38 |
| 199 | 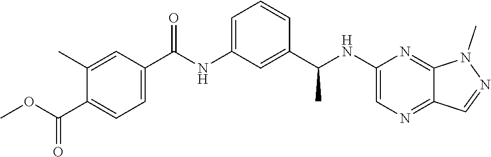 [SMILES: COC(=O)c1ccc(C(=O)Nc2cccc([C@H](C)Nc3cnc4cnn(C)c4n3)c2)cc1C] | 444.48 | 445.2 |
| 200 | 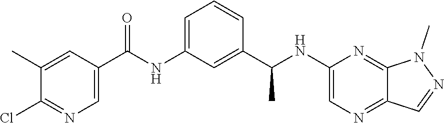 [SMILES: Cc1cc(C(=O)Nc2cccc([C@H](C)Nc3cnc4cnn(C)c4n3)c2)cnc1Cl] | 421.88 | 422.7 |
| 201 | 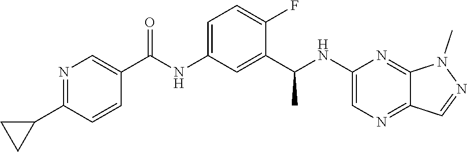 [SMILES: C[C@H](Nc1cnc2cnn(C)c2n1)c1cc(NC(=O)c2ccc(C3CC3)nc2)ccc1F] | 431.46 | 432.3 |
| 202 | 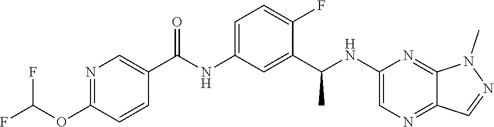 [SMILES: C[C@H](Nc1cnc2cnn(C)c2n1)c1cc(NC(=O)c2ccc(OC(F)F)nc2)ccc1F] | 457.41 | 458.2 |
| 203 | 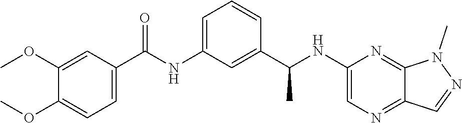 [SMILES: COc1ccc(C(=O)Nc2cccc([C@H](C)Nc3cnc4cnn(C)c4n3)c2)cc1OC] | 432.47 | 433.1 |
| 204 | 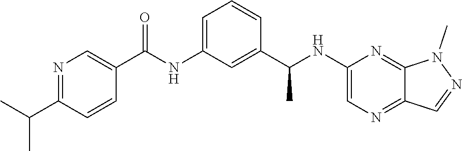 [SMILES: CC(C)c1ccc(C(=O)Nc2cccc([C@H](C)Nc3cnc4cnn(C)c4n3)c2)cn1] | 415.49 | 416.2 |
| 205 | 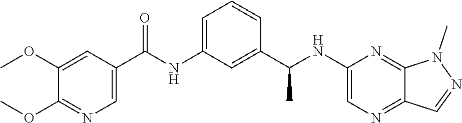 [SMILES: COc1cc(C(=O)Nc2cccc([C@H](C)Nc3cnc4cnn(C)c4n3)c2)cnc1OC] | 433.46 | 434.3 |
| 206 | 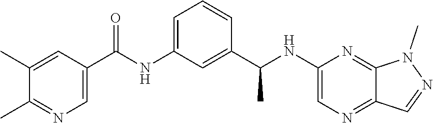 [SMILES: Cc1cc(C(=O)Nc2cccc([C@H](C)Nc3cnc4cnn(C)c4n3)c2)cnc1C] | 401.46 | 402.3 |
| 207 | 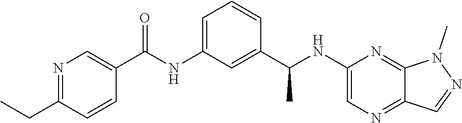 [SMILES: CCc1ccc(C(=O)Nc2cccc([C@H](C)Nc3cnc4cnn(C)c4n3)c2)cn1] | 401.46 | 402.2 |
| 208 | 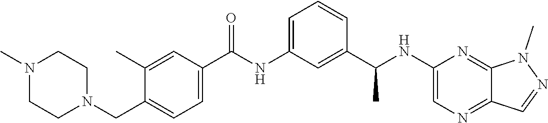 [SMILES: Cc1cc(C(=O)Nc2cccc([C@H](C)Nc3cnc4cnn(C)c4n3)c2)ccc1CN1CCN(C)CC1] | 498.62 | 499.6 |
| 209 | 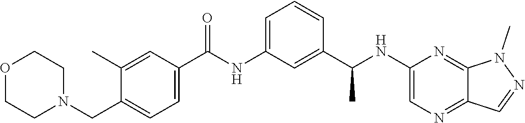 [SMILES: Cc1cc(C(=O)Nc2cccc([C@H](C)Nc3cnc4cnn(C)c4n3)c2)ccc1CN1CCOCC1] | 485.58 | 486.9 |
| 210 | 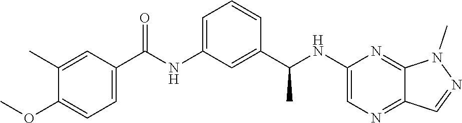 [SMILES: COc1ccc(C(=O)Nc2cccc([C@H](C)Nc3cnc4cnn(C)c4n3)c2)cc1C] | 416.47 | 417.6 |
| 211 | 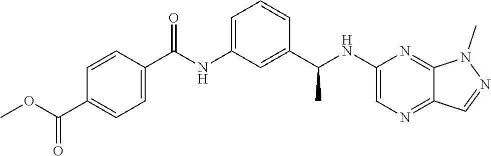 [SMILES: COC(=O)c1ccc(C(=O)Nc2cccc([C@H](C)Nc3cnc4cnn(C)c4n3)c2)cc1] | 430.46 | 432.1 |
| 212 | 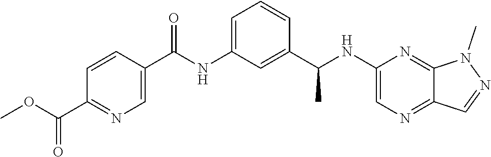 [SMILES: COC(=O)c1ccc(C(=O)Nc2cccc([C@H](C)Nc3cnc4cnn(C)c4n3)c2)cn1] | 431.45 | 433.03 |
| 213 | 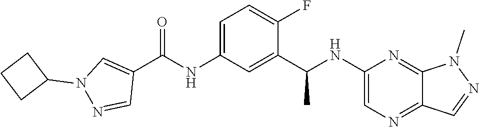 [SMILES: C[C@H](Nc1cnc2cnn(C)c2n1)c1cc(NC(=O)c2cnn(C3CCC3)c2)ccc1F] | 434.47 | 435.3 |
| 214 | 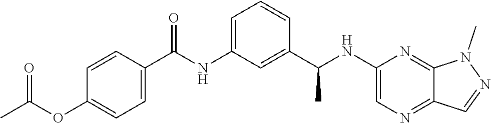 [SMILES: CC(=O)Oc1ccc(C(=O)Nc2cccc([C@H](C)Nc3cnc4cnn(C)c4n3)c2)cc1] | 430.46 | 431.1 |
| 215 | 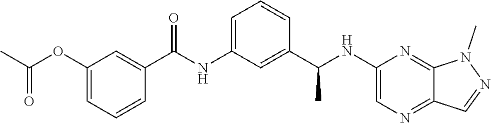 [SMILES: CC(=O)Oc1cccc(C(=O)Nc2cccc([C@H](C)Nc3cnc4cnn(C)c4n3)c2)c1] | 430.46 | 431.1 |
| 216 | 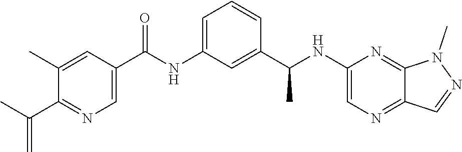 [SMILES: C=C(C)c1ncc(C(=O)Nc2cccc([C@H](C)Nc3cnc4cnn(C)c4n3)c2)cc1C] | 427.5 | 428.3 |
| 217 | 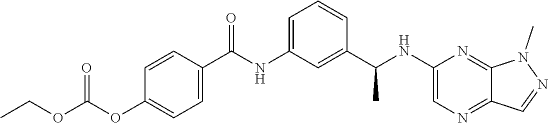 [SMILES: CCOC(=O)Oc1ccc(C(=O)Nc2cccc([C@H](C)Nc3cnc4cnn(C)c4n3)c2)cc1] | 460.48 | 461.1 |
| 218 | 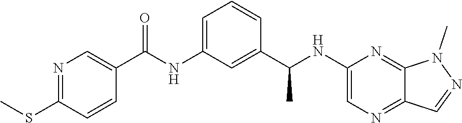 [SMILES: CSc1ccc(C(=O)Nc2cccc([C@H](C)Nc3cnc4cnn(C)c4n3)c2)cn1] | 419.5 | 420.5 |
| 219 | 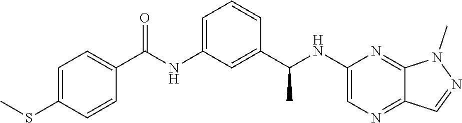 [SMILES: CSc1ccc(C(=O)Nc2cccc([C@H](C)Nc3cnc4cnn(C)c4n3)c2)cc1] | 418.51 | 419.5 |
| 220 | 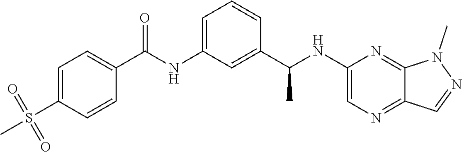 [SMILES: C[C@H](Nc1cnc2cnn(C)c2n1)c1cccc(NC(=O)c2ccc(S(C)(=O)=O)cc2)c1] | 450.51 | 451.1 |
| 221 | 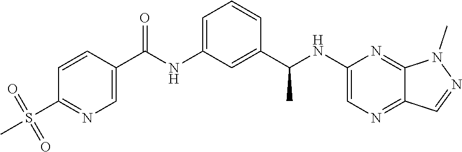 [SMILES: C[C@H](Nc1cnc2cnn(C)c2n1)c1cccc(NC(=O)c2ccc(S(C)(=O)=O)nc2)c1] | 451.5 | 452.6 |
| 222 | 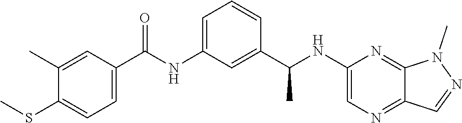 [SMILES: CSc1ccc(C(=O)Nc2cccc([C@H](C)Nc3cnc4cnn(C)c4n3)c2)cc1C] | 432.54 | 433.6 |
| 223 | 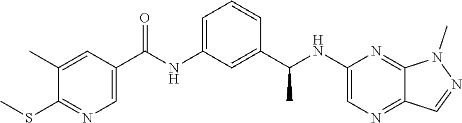 [SMILES: CSc1ncc(C(=O)Nc2cccc([C@H](C)Nc3cnc4cnn(C)c4n3)c2)cc1C] | 433.53 | 435 |
| 224 | 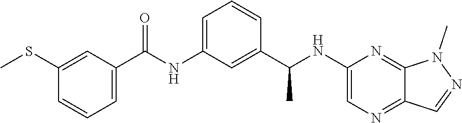 [SMILES: CSc1cccc(C(=O)Nc2cccc([C@H](C)Nc3cnc4cnn(C)c4n3)c2)c1] | 418.51 | 419.6 |
| 225 | 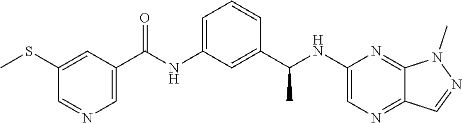 [SMILES: CSc1cncc(C(=O)Nc2cccc([C@H](C)Nc3cnc4cnn(C)c4n3)c2)c1] | 419.5 | 420.5 |
| 226 | 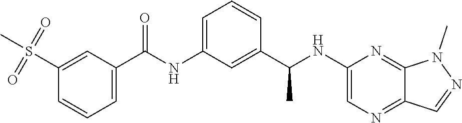 [SMILES: C[C@H](Nc1cnc2cnn(C)c2n1)c1cccc(NC(=O)c2cccc(S(C)(=O)=O)c2)c1] | 450.51 | 452 |
| 227 | 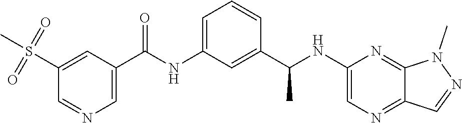 [SMILES: C[C@H](Nc1cnc2cnn(C)c2n1)c1cccc(NC(=O)c2cncc(S(C)(=O)=O)c2)c1] | 451.5 | 452.8 |
| 228 | 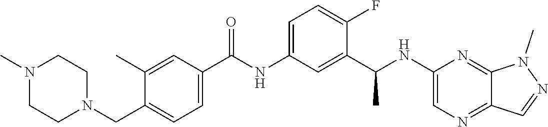 [SMILES: Cc1cc(C(=O)Nc2ccc(F)c([C@H](C)Nc3cnc4cnn(C)c4n3)c2)ccc1CN1CCN(C)CC1] | 516.61 | 517.3 |
| 229 | 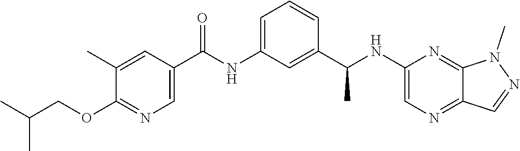 [SMILES: Cc1cc(C(=O)Nc2cccc([C@H](C)Nc3cnc4cnn(C)c4n3)c2)cnc1OCC(C)C] | 459.54 | 460.3 |
| 230 | 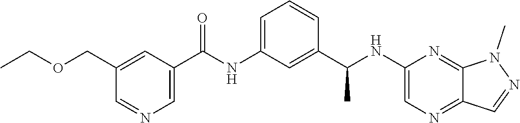 [SMILES: CCOCc1cncc(C(=O)Nc2cccc([C@H](C)Nc3cnc4cnn(C)c4n3)c2)c1] | 431.49 | 432.2 |
| 231 | 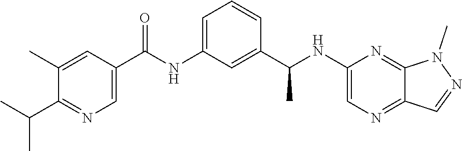 [SMILES: Cc1cc(C(=O)Nc2cccc([C@H](C)Nc3cnc4cnn(C)c4n3)c2)cnc1C(C)C] | 429.52 | 430.2 |
| 232 | 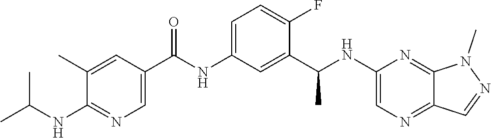 [SMILES: Cc1cc(C(=O)Nc2ccc(F)c([C@H](C)Nc3cnc4cnn(C)c4n3)c2)cnc1NC(C)C] | 462.52 | 463.3 |
| 233 | 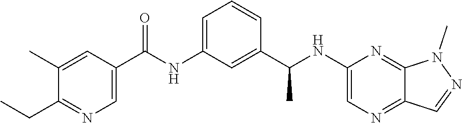 [SMILES: CCc1ncc(C(=O)Nc2cccc([C@H](C)Nc3cnc4cnn(C)c4n3)c2)cc1C] | 415.49 | 416.3 |
| 234 | 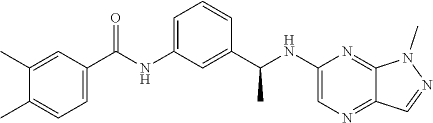 [SMILES: Cc1ccc(C(=O)Nc2cccc([C@H](C)Nc3cnc4cnn(C)c4n3)c2)cc1C] | 400.48 | 401.1 |
| 235 | 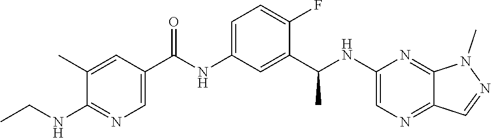 [SMILES: CCNc1ncc(C(=O)Nc2ccc(F)c([C@H](C)Nc3cnc4cnn(C)c4n3)c2)cc1C] | 448.5 | 449.2 |
| 236 | 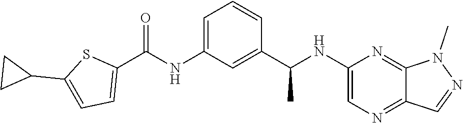 | 452.96 | 453.5 |
| 237 | 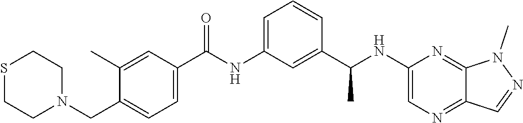 [SMILES: Cc1cc(C(=O)Nc2cccc([C@H](C)Nc3cnc4cnn(C)c4n3)c2)ccc1CN1CCSCC1] | 501.65 | 502.6 |
| 238 | 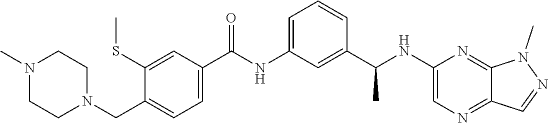 [SMILES: CSc1cc(C(=O)Nc2cccc([C@H](C)Nc3cnc4cnn(C)c4n3)c2)ccc1CN1CCN(C)CC1] | 530.69 | 531.6 |
| 239 | 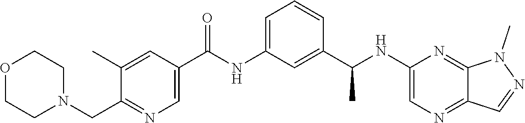 [SMILES: Cc1cc(C(=O)Nc2cccc([C@H](C)Nc3cnc4cnn(C)c4n3)c2)cnc1CN1CCOCC1] | 486.57 | 487.6 |
| 240 | 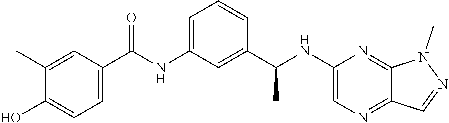 [SMILES: Cc1cc(C(=O)Nc2cccc([C@H](C)Nc3cnc4cnn(C)c4n3)c2)ccc1O] | 402.45 | 403.6 |
| 241 | 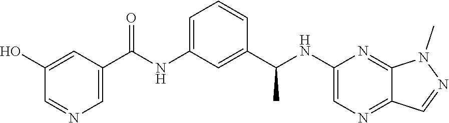 [SMILES: C[C@H](Nc1cnc2cnn(C)c2n1)c1cccc(NC(=O)c2cncc(O)c2)c1] | 389.41 | 390.1 |
| 242 | 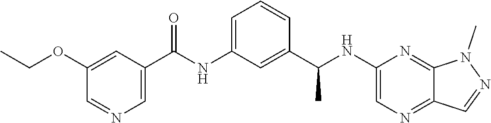 [SMILES: CCOc1cncc(C(=O)Nc2cccc([C@H](C)Nc3cnc4cnn(C)c4n3)c2)c1] | 417.46 | 418.4 |
| 243 | 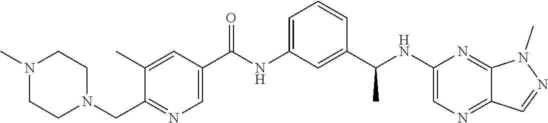 [SMILES: Cc1cc(C(=O)Nc2cccc([C@H](C)Nc3cnc4cnn(C)c4n3)c2)cnc1CN1CCN(C)CC1] | 499.61 | 500.6 |
| 244 | 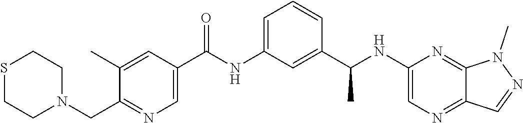 [SMILES: Cc1cc(C(=O)Nc2cccc([C@H](C)Nc3cnc4cnn(C)c4n3)c2)cnc1CN1CCSCC1] | 502.63 | 503.6 |
| 245 | 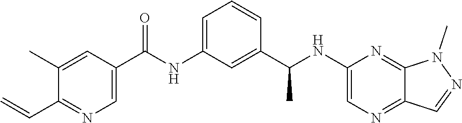 [SMILES: C=Cc1ncc(C(=O)Nc2cccc([C@H](C)Nc3cnc4cnn(C)c4n3)c2)cc1C] | 413.47 | 414.2 |
| 246 | 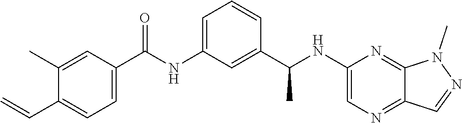 [SMILES: C=Cc1ccc(C(=O)Nc2cccc([C@H](C)Nc3cnc4cnn(C)c4n3)c2)cc1C] | 412.49 | 413.3 |
| 247 | 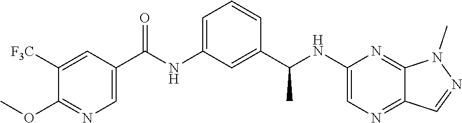 [SMILES: COc1ncc(C(=O)Nc2cccc([C@H](C)Nc3cnc4cnn(C)c4n3)c2)cc1C(F)(F)F] | 471.43 | 472.2 |
| 248 | 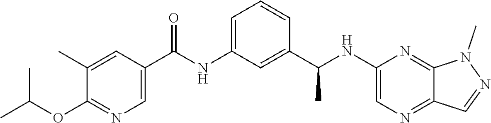 [SMILES: Cc1cc(C(=O)Nc2cccc([C@H](C)Nc3cnc4cnn(C)c4n3)c2)cnc1OC(C)C] | 445.52 | 446.3 |
| 249 | 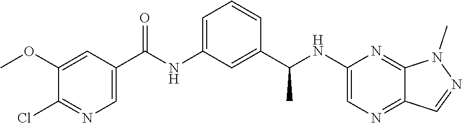 [SMILES: COc1cc(C(=O)Nc2cccc([C@H](C)Nc3cnc4cnn(C)c4n3)c2)cnc1Cl] | 437.88 | 438.1 |
| 250 | 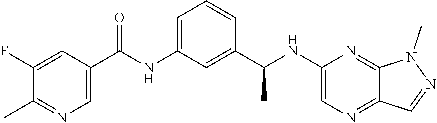 [SMILES: Cc1ncc(C(=O)Nc2cccc([C@H](C)Nc3cnc4cnn(C)c4n3)c2)cc1F] | 405.43 | 406.6 |
| 251 | 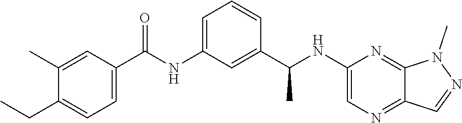 [SMILES: CCc1ccc(C(=O)Nc2cccc([C@H](C)Nc3cnc4cnn(C)c4n3)c2)cc1C] | 414.5 | 415.3 |
| 252 | 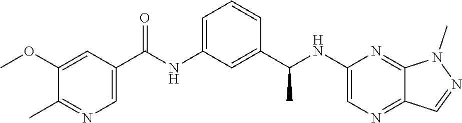 [SMILES: COc1cc(C(=O)Nc2cccc([C@H](C)Nc3cnc4cnn(C)c4n3)c2)cnc1C] | 417.46 | 418.2 |
| 253 | 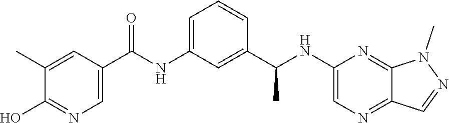 [SMILES: Cc1cc(C(=O)Nc2cccc([C@H](C)Nc3cnc4cnn(C)c4n3)c2)cnc1O] | 403.44 | 404.2 |
| 254 | 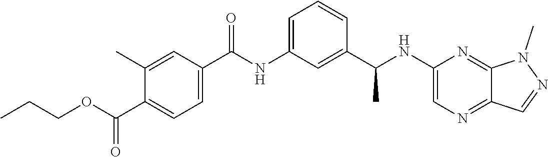 [SMILES: CCCOC(=O)c1ccc(C(=O)Nc2cccc([C@H](C)Nc3cnc4cnn(C)c4n3)c2)cc1C] | 472.54 | 473.3 |
| 255 | 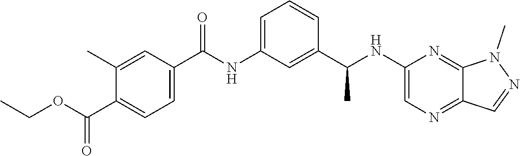 [SMILES: CCOC(=O)c1ccc(C(=O)Nc2cccc([C@H](C)Nc3cnc4cnn(C)c4n3)c2)cc1C] | 458.51 | 459.2 |
| 256 | 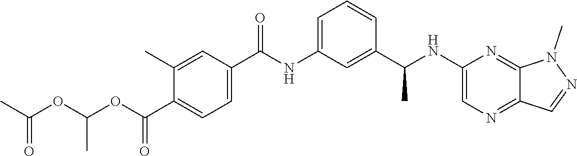 [SMILES: CC(=O)OC(C)OC(=O)c1ccc(C(=O)Nc2cccc([C@H](C)Nc3cnc4cnn(C)c4n3)c2)cc1C] | 516.55 | 517.2 |
| 257 | 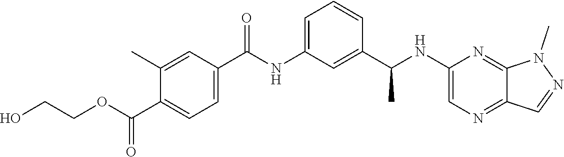 [SMILES: Cc1cc(C(=O)Nc2cccc([C@H](C)Nc3cnc4cnn(C)c4n3)c2)ccc1C(=O)OCCO] | 474.51 | 475.3 |
| 258 | 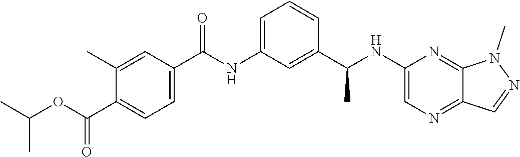 [SMILES: Cc1cc(C(=O)Nc2cccc([C@H](C)Nc3cnc4cnn(C)c4n3)c2)ccc1C(=O)OC(C)C] | 472.54 | 473.3 |
| 259 | 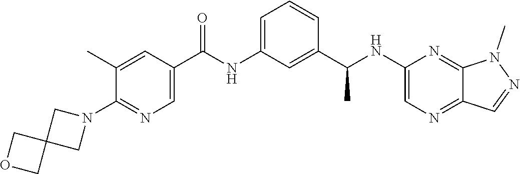 [SMILES: Cc1cc(C(=O)Nc2cccc([C@H](C)Nc3cnc4cnn(C)c4n3)c2)cnc1N1CC2(COC2)C1] | 484.55 | 485.4 |
| 260 | 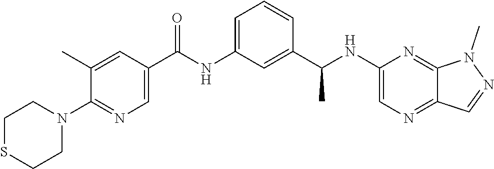 [SMILES: Cc1cc(C(=O)Nc2cccc([C@H](C)Nc3cnc4cnn(C)c4n3)c2)cnc1N1CCSCC1] | 488.61 | 489.5 |
| 261 | 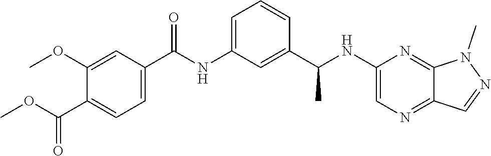 [SMILES: COC(=O)c1ccc(C(=O)Nc2cccc([C@H](C)Nc3cnc4cnn(C)c4n3)c2)cc1OC] | 460.48 | 461.5 |
| 262 | 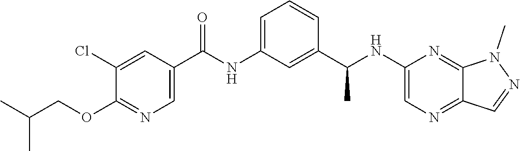 [SMILES: CC(C)COc1ncc(C(=O)Nc2cccc([C@H](C)Nc3cnc4cnn(C)c4n3)c2)cc1Cl] | 479.96 | 480.2 |
| 263 | 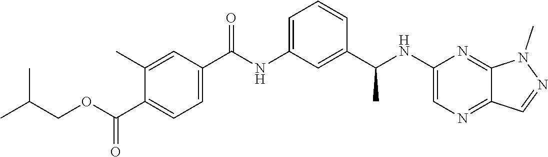 [SMILES: Cc1cc(C(=O)Nc2cccc([C@H](C)Nc3cnc4cnn(C)c4n3)c2)ccc1C(=O)OCC(C)C] | 486.56 | 487.3 |
| 264 | 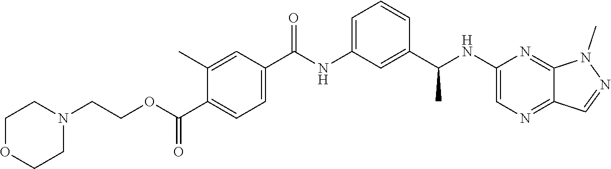 [SMILES: Cc1cc(C(=O)Nc2cccc([C@H](C)Nc3cnc4cnn(C)c4n3)c2)ccc1C(=O)OCCN1CCOCC1] | 543.62 | 544.3 |
| 265 | 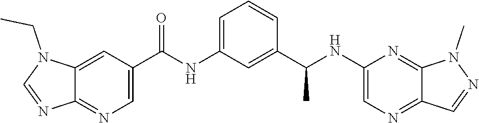 [SMILES: CCn1cnc2ncc(C(=O)Nc3cccc([C@H](C)Nc4cnc5cnn(C)c5n4)c3)cc21] | 441.49 | 442.3 |
| 266 | 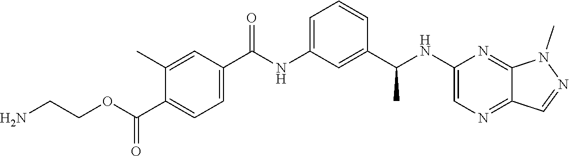 [SMILES: Cc1cc(C(=O)Nc2cccc([C@H](C)Nc3cnc4cnn(C)c4n3)c2)ccc1C(=O)OCCN] | 473.53 | 474.2 |
| 267 | 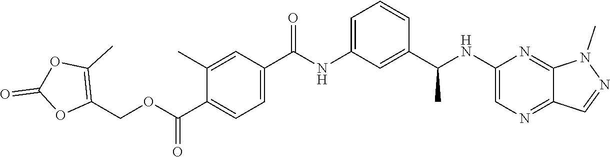 [SMILES: Cc1cc(C(=O)Nc2cccc([C@H](C)Nc3cnc4cnn(C)c4n3)c2)ccc1C(=O)OCc1oc(=O)oc1C] | 542.54 | 543.3 |
| 268 | 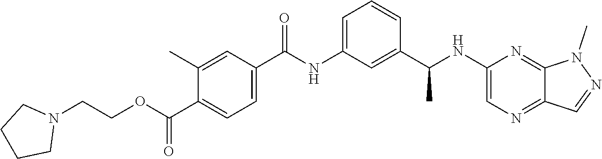 [SMILES: Cc1cc(C(=O)Nc2cccc([C@H](C)Nc3cnc4cnn(C)c4n3)c2)ccc1C(=O)OCCN1CCCC1] | 527.62 | 528.4 |
| 269 | 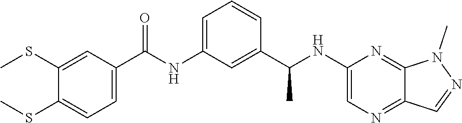 [SMILES: CSc1ccc(C(=O)Nc2cccc([C@H](C)Nc3cnc4cnn(C)c4n3)c2)cc1SC] | 464.61 | 465.1 |
| 270 | 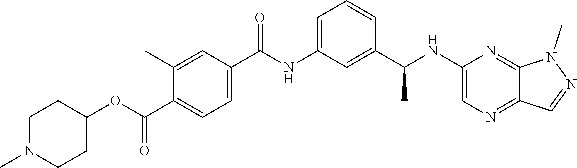 [SMILES: Cc1cc(C(=O)Nc2cccc([C@H](C)Nc3cnc4cnn(C)c4n3)c2)ccc1C(=O)OC1CCN(C)CC1] | 527.62 | 528.3 |
| 271 | 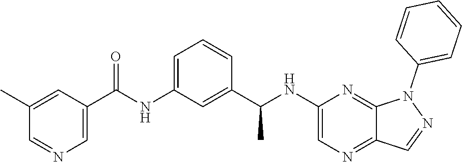 [SMILES: Cc1cncc(C(=O)Nc2cccc([C@H](C)Nc3cnc4cnn(-c5ccccc5)c4n3)c2)c1] | 449.51 | 450.1 |
| 272 | 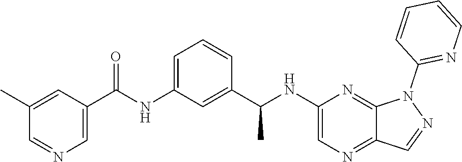 [SMILES: Cc1cncc(C(=O)Nc2cccc([C@H](C)Nc3cnc4cnn(-c5ccccn5)c4n3)c2)c1] | 450.49 | 451.1 |
| 273 | 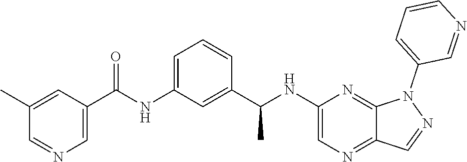 [SMILES: Cc1cncc(C(=O)Nc2cccc([C@H](C)Nc3cnc4cnn(-c5cccnc5)c4n3)c2)c1] | 450.49 | 451.2 |
| 274 | 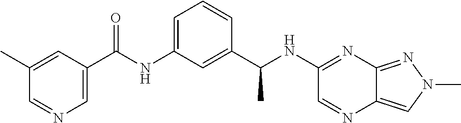 | 387.44 | 388.22 |
| 275 | 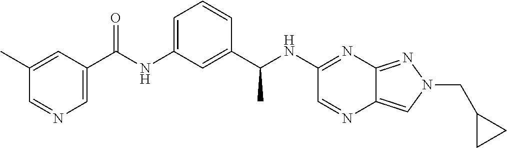 [SMILES: Cc1cncc(C(=O)Nc2cccc([C@H](C)Nc3cnc4cn(CC5CC5)nc4n3)c2)c1] | 427.51 | 428.30 |
| 276 | 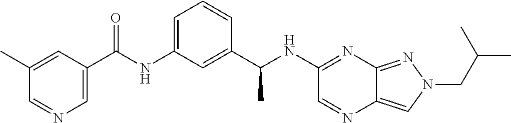 [SMILES: Cc1cncc(C(=O)Nc2cccc([C@H](C)Nc3cnc4cn(CC(C)C)nc4n3)c2)c1] | 429.53 | 430.30 |
| 277 | 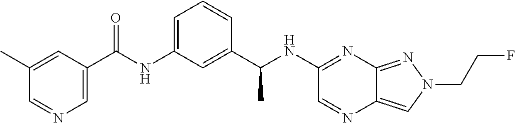 [SMILES: Cc1cncc(C(=O)Nc2cccc([C@H](C)Nc3cnc4cn(CCF)nc4n3)c2)c1] | 419.46 | 420.25 |
| 278 | 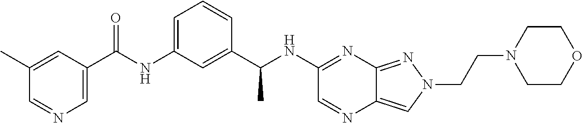 [SMILES: Cc1cncc(C(=O)Nc2cccc([C@H](C)Nc3cnc4cn(CCN5CCOCC5)nc4n3)c2)c1] | 486.58 | 487.30 |
| 279 | 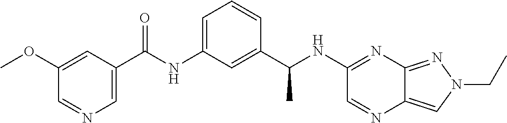 [SMILES: CCn1cc2ncc(N[C@@H](C)c3cccc(NC(=O)c4cncc(OC)c4)c3)nc2n1] | 417.47 | 418.20 |
| 280 | 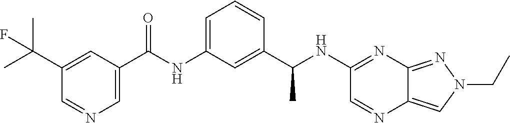 [SMILES: CCn1cc2ncc(N[C@@H](C)c3cccc(NC(=O)c4cncc(C(C)(C)F)c4)c3)nc2n1] | 447.52 | 448.30 |
| 281 | 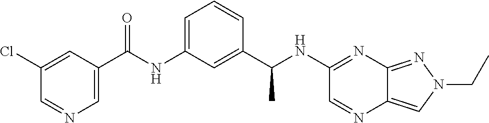 [SMILES: CCn1cc2ncc(N[C@@H](C)c3cccc(NC(=O)c4cncc(Cl)c4)c3)nc2n1] | 421.89 | 422.20 |
| 282 | 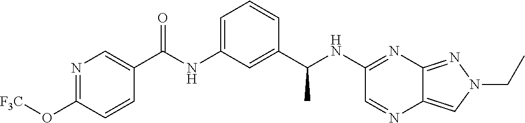 [SMILES: CCn1cc2ncc(N[C@@H](C)c3cccc(NC(=O)c4ccc(OC(F)(F)F)nc4)c3)nc2n1] | 471.44 | 472.25 |
| 283 | 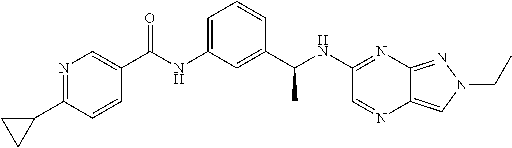 [SMILES: CCn1cc2ncc(N[C@@H](C)c3cccc(NC(=O)c4ccc(C5CC5)nc4)c3)nc2n1] | 427.51 | 428.00 |
| 284 | 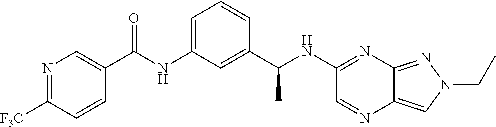 [SMILES: CCn1cc2ncc(N[C@@H](C)c3cccc(NC(=O)c4ccc(C(F)(F)F)nc4)c3)nc2n1] | 455.45 | 456.20 |
| 285 | 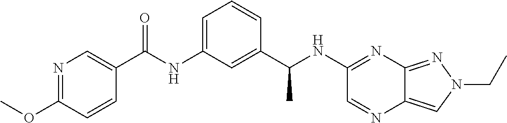 [SMILES: CCn1cc2ncc(N[C@@H](C)c3cccc(NC(=O)c4ccc(OC)nc4)c3)nc2n1] | 417.47 | 418.20 |
| 286 | 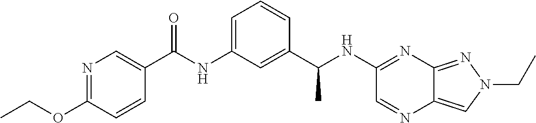 [SMILES: CCOc1ccc(C(=O)Nc2cccc([C@H](C)Nc3cnc4cn(CC)nc4n3)c2)cn1] | 431.5 | 432.30 |
| 287 | 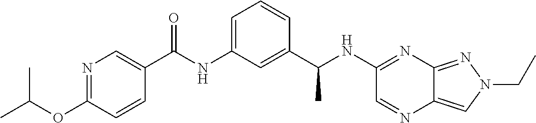 [SMILES: CCn1cc2ncc(N[C@@H](C)c3cccc(NC(=O)c4ccc(OC(C)C)nc4)c3)nc2n1] | 445.53 | 446.25 |
| 288 | 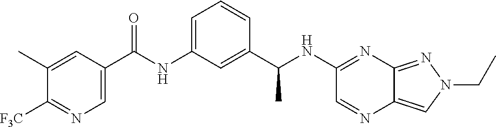 [SMILES: CCn1cc2ncc(N[C@@H](C)c3cccc(NC(=O)c4cnc(C(F)(F)F)c(C)c4)c3)nc2n1] | 469.47 | 470.20 |
| 289 | 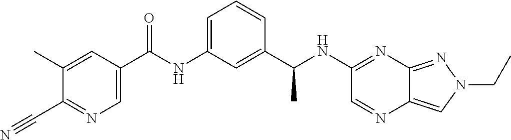 [SMILES: CCn1cc2ncc(N[C@@H](C)c3cccc(NC(=O)c4cnc(C#N)c(C)c4)c3)nc2n1] | 426.48 | 427.25 |
| 290 | 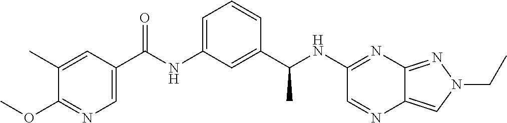 [SMILES: CCn1cc2ncc(N[C@@H](C)c3cccc(NC(=O)c4cnc(OC)c(C)c4)c3)nc2n1] | 431.5 | 432.20 |
| 291 | 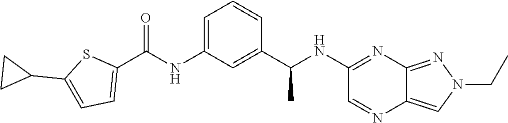 [SMILES: CCn1cc2ncc(N[C@@H](C)c3cccc(NC(=O)c4ccc(C5CC5)s4)c3)nc2n1] | 432.55 | 433.10 |
| 292 | 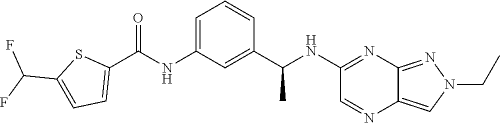 [SMILES: CCn1cc2ncc(N[C@@H](C)c3cccc(NC(=O)c4ccc(C(F)F)s4)c3)nc2n1] | 442.49 | 443.20 |
| 293 | 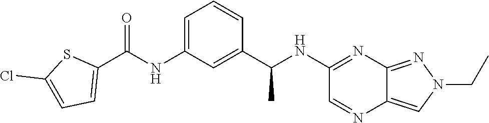 [SMILES: CCn1cc2ncc(N[C@@H](C)c3cccc(NC(=O)c4ccc(Cl)s4)c3)nc2n1] | 426.92 | 427.20 |
| 294 | 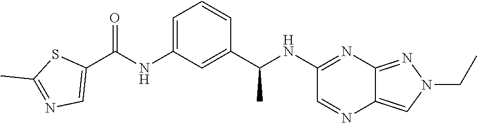 [SMILES: CCn1cc2ncc(N[C@@H](C)c3cccc(NC(=O)c4cnc(C)s4)c3)nc2n1] | 407.5 | 407.90 |
| 295 | 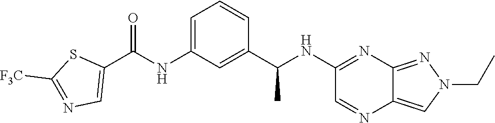 [SMILES: CCn1cc2ncc(N[C@@H](C)c3cccc(NC(=O)c4cnc(C(F)(F)F)s4)c3)nc2n1] | 461.47 | 461.90 |
| 296 | 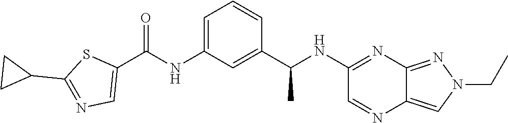 [SMILES: CCn1cc2ncc(N[C@@H](C)c3cccc(NC(=O)c4cnc(C5CC5)s4)c3)nc2n1] | 433.53 | 434.25 |
| 297 | 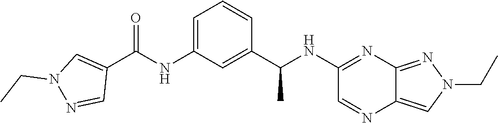 [SMILES: CCn1cc(C(=O)Nc2cccc([C@H](C)Nc3cnc4cn(CC)nc4n3)c2)cn1] | 404.48 | 405.30 |
| 298 | 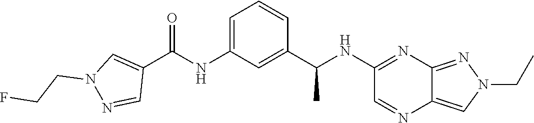 [SMILES: CCn1cc2ncc(N[C@@H](C)c3cccc(NC(=O)c4cnn(CCF)c4)c3)nc2n1] | 422.47 | 423.20 |
| 299 | 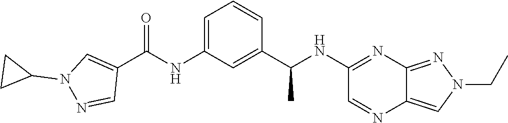 [SMILES: CCn1cc2ncc(N[C@@H](C)c3cccc(NC(=O)c4cnn(C5CC5)c4)c3)nc2n1] | 416.49 | 417.30 |
| 300 | 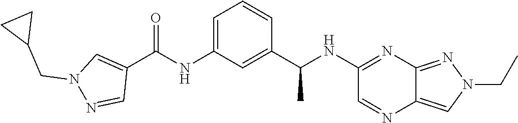 [SMILES: CCn1cc2ncc(N[C@@H](C)c3cccc(NC(=O)c4cnn(CC5CC5)c4)c3)nc2n1] | 430.52 | 431.20 |
| 301 | 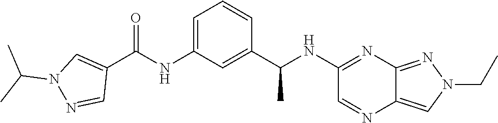 [SMILES: CCn1cc2ncc(N[C@@H](C)c3cccc(NC(=O)c4cnn(C(C)C)c4)c3)nc2n1] | 418.51 | 419.20 |
| 302 | 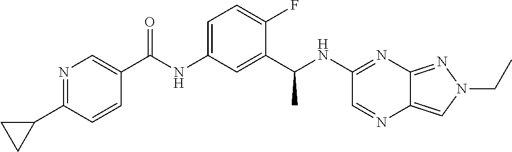 [SMILES: CCn1cc2ncc(N[C@@H](C)c3cc(NC(=O)c4ccc(C5CC5)nc4)ccc3F)nc2n1] | 445.5 | 446.20 |
| 303 | 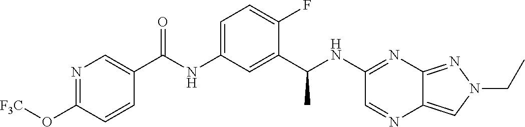 [SMILES: CCn1cc2ncc(N[C@@H](C)c3cc(NC(=O)c4ccc(OC(F)(F)F)nc4)ccc3F)nc2n1] | 489.43 | 490.20 |
| 304 | 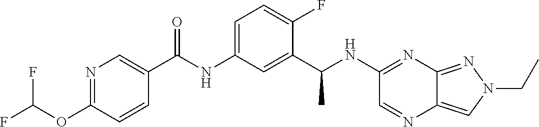 [SMILES: CCn1cc2ncc(N[C@@H](C)c3cc(NC(=O)c4ccc(OC(F)F)nc4)ccc3F)nc2n1] | 471.44 | 472.25 |
| 305 | 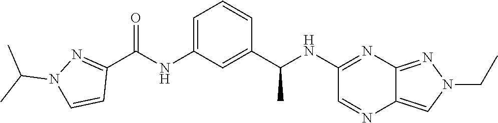 [SMILES: CCn1cc2ncc(N[C@@H](C)c3cccc(NC(=O)c4ccn(C(C)C)n4)c3)nc2n1] | 418.51 | 419.25 |
| 306 | 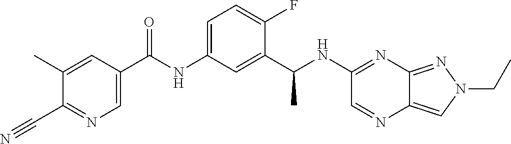 [SMILES: CCn1cc2ncc(N[C@@H](C)c3cc(NC(=O)c4cnc(C#N)c(C)c4)ccc3F)nc2n1] | 444.47 | 445.20 |
| 307 | 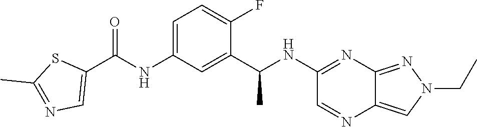 [SMILES: CCn1cc2ncc(N[C@@H](C)c3cc(NC(=O)c4cnc(C)s4)ccc3F)nc2n1] | 425.49 | 426.20 |
| 308 | 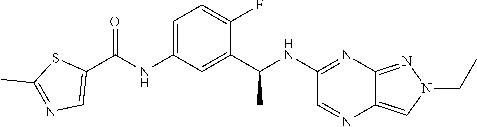 [SMILES: CCn1cc2ncc(N[C@@H](C)c3cc(NC(=O)c4cnc(C)s4)ccc3F)nc2n1] | 479.46 | 480.10 |
| 309 | 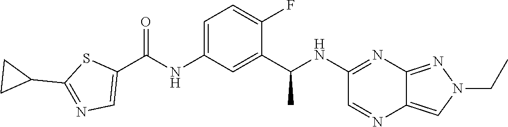 [SMILES: CCn1cc2ncc(N[C@@H](C)c3cc(NC(=O)c4cnc(C5CC5)s4)ccc3F)nc2n1] | 451.52 | 452.25 |
| 310 | 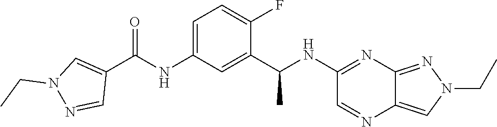 [SMILES: CCn1cc(C(=O)Nc2ccc(F)c([C@H](C)Nc3cnc4cn(CC)nc4n3)c2)cn1] | 422.47 | 423.10 |
| 311 | 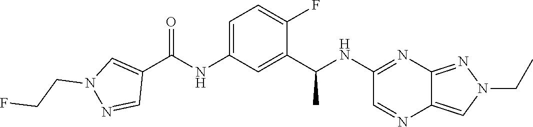 [SMILES: CCn1cc2ncc(N[C@@H](C)c3cc(NC(=O)c4cnn(CCF)c4)ccc3F)nc2n1] | 440.46 | 441.30 |
| 312 | 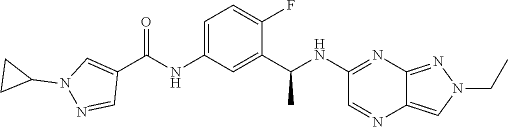 [SMILES: CCn1cc2ncc(N[C@@H](C)c3cc(NC(=O)c4cnn(C5CC5)c4)ccc3F)nc2n1] | 434.48 | 435.30 |
| 313 | 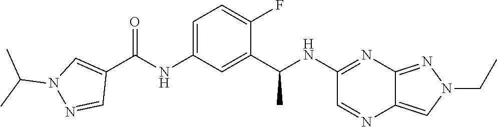 [SMILES: CCn1cc2ncc(N[C@@H](C)c3cc(NC(=O)c4cnn(C(C)C)c4)ccc3F)nc2n1] | 436.5 | 437.30 |
| 314 | 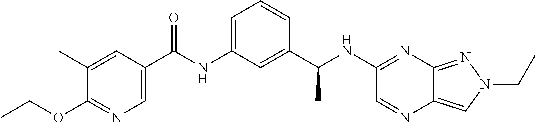 [SMILES: CCOc1ncc(C(=O)Nc2cccc([C@H](C)Nc3cnc4cn(CC)nc4n3)c2)cc1C] | 445.52 | 446.3 |
| 315 | 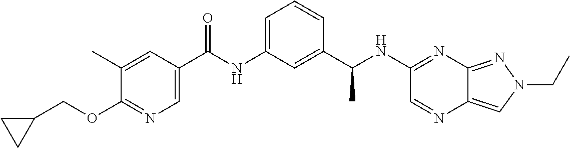 [SMILES: CCn1cc2ncc(N[C@@H](C)c3cccc(NC(=O)c4cnc(OCC5CC5)c(C)c4)c3)nc2n1] | 471.55 | 472.3 |
| 316 | 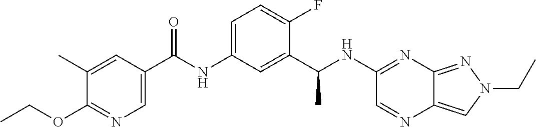 [SMILES: CCOc1ncc(C(=O)Nc2ccc(F)c([C@H](C)Nc3cnc4cn(CC)nc4n3)c2)cc1C] | 463.51 | 464.2 |
| 317 | 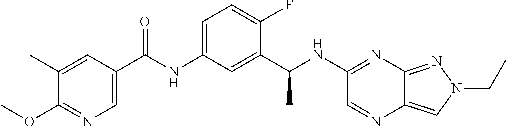 [SMILES: CCn1cc2ncc(N[C@@H](C)c3cc(NC(=O)c4cnc(OC)c(C)c4)ccc3F)nc2n1] | 449.48 | 450.3 |
| 318 | 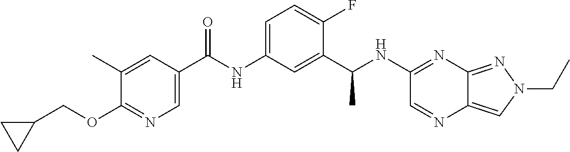 [SMILES: CCn1cc2ncc(N[C@@H](C)c3cc(NC(=O)c4cnc(OCC5CC5)c(C)c4)ccc3F)nc2n1] | 489.54 | 490.3 |
| 319 | 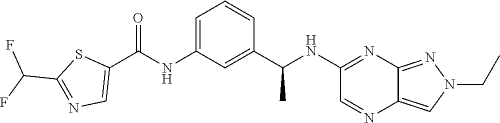 [SMILES: CCn1cc2ncc(N[C@@H](C)c3cccc(NC(=O)c4cnc(C(F)F)s4)c3)nc2n1] | 443.47 | 444.3 |
| 320 | 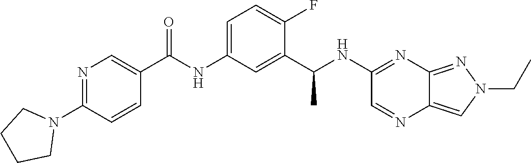 [SMILES: CCn1cc2ncc(N[C@@H](C)c3cc(NC(=O)c4ccc(N5CCCC5)nc4)ccc3F)nc2n1] | 474.53 | 475.3 |
| 321 | 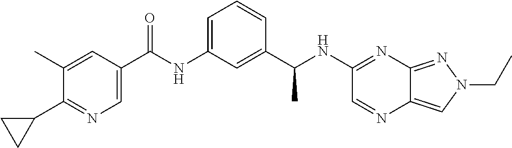 [SMILES: CCn1cc2ncc(N[C@@H](C)c3cccc(NC(=O)c4cnc(C5CC5)c(C)c4)c3)nc2n1] | 441.53 | 442.3 |
| 322 | 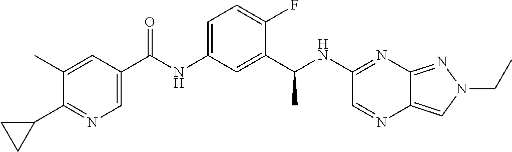 [SMILES: CCn1cc2ncc(N[C@@H](C)c3cc(NC(=O)c4cnc(C5CC5)c(C)c4)ccc3F)nc2n1] | 459.52 | 460.4 |
| 323 | 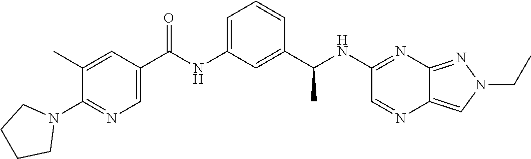 [SMILES: CCn1cc2ncc(N[C@@H](C)c3cccc(NC(=O)c4cnc(N5CCCC5)c(C)c4)c3)nc2n1] | 470.57 | 471.3 |
| 324 | 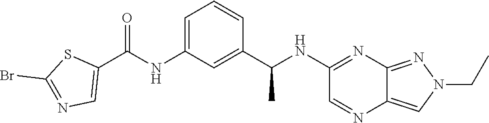 [SMILES: CCn1cc2ncc(N[C@@H](C)c3cccc(NC(=O)c4cnc(Br)s4)c3)nc2n1] | 472.36 | 474.1 |
| 325 | 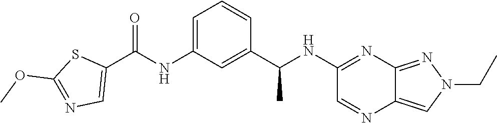 [SMILES: CCn1cc2ncc(N[C@@H](C)c3cccc(NC(=O)c4cnc(OC)s4)c3)nc2n1] | 423.49 | 424.2 |
| 326 | 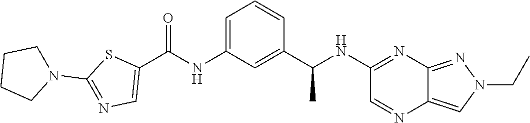 [SMILES: CCn1cc2ncc(N[C@@H](C)c3cccc(NC(=O)c4cnc(N5CCCC5)s4)c3)nc2n1] | 462.57 | 463.3 |
| 327 | 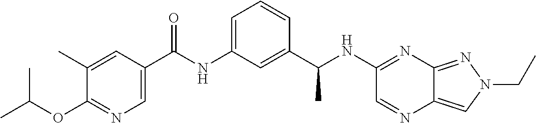 [SMILES: CCn1cc2ncc(N[C@@H](C)c3cccc(NC(=O)c4cnc(OC(C)C)c(C)c4)c3)nc2n1] | 459.54 | 460.3 |
| 328 | 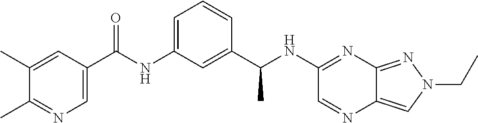 [SMILES: CCn1cc2ncc(N[C@@H](C)c3cccc(NC(=O)c4cnc(C)c(C)c4)c3)nc2n1] | 415.49 | 416.3 |
| 329 | 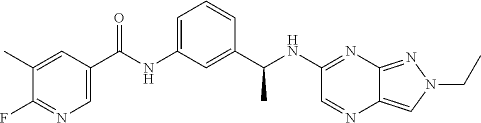 [SMILES: CCn1cc2ncc(N[C@@H](C)c3cccc(NC(=O)c4cnc(F)c(C)c4)c3)nc2n1] | 419.45 | 420.9 |
| 330 | 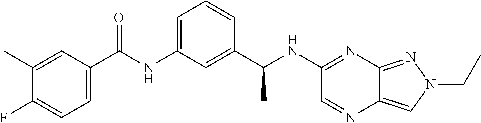 [SMILES: CCn1cc2ncc(N[C@@H](C)c3cccc(NC(=O)c4ccc(F)c(C)c4)c3)nc2n1] | 418.47 | 420.16 |
| 331 | 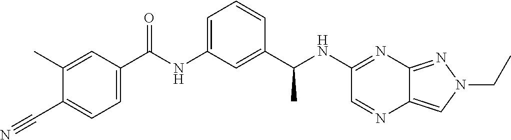 [SMILES: CCn1cc2ncc(N[C@@H](C)c3cccc(NC(=O)c4ccc(C#N)c(C)c4)c3)nc2n1] | 425.49 | 426.9 |
| 332 | 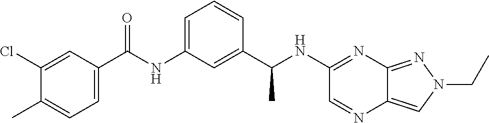 [SMILES: CCn1cc2ncc(N[C@@H](C)c3cccc(NC(=O)c4ccc(C)c(Cl)c4)c3)nc2n1] | 434.92 | 435.3 |
| 333 | 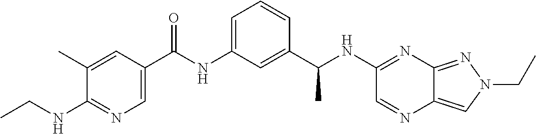 [SMILES: CCNc1ncc(C(=O)Nc2cccc([C@H](C)Nc3cnc4cn(CC)nc4n3)c2)cc1C] | 444.53 | 445.7 |
| 334 | 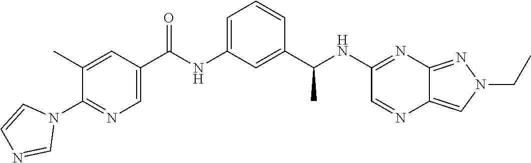 [SMILES: CCn1cc2ncc(N[C@@H](C)c3cccc(NC(=O)c4cnc(-n5ccnc5)c(C)c4)c3)nc2n1] | 467.53 | 468.3 |
| 335 | 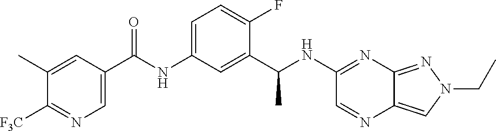 [SMILES: CCn1cc2ncc(N[C@@H](C)c3cc(NC(=O)c4cnc(C(F)(F)F)c(C)c4)ccc3F)nc2n1] | 487.45 | 488.2 |
| 336 | 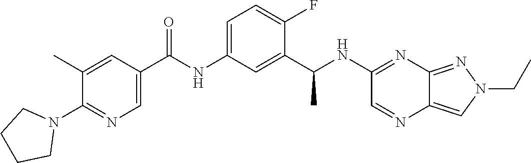 [SMILES: CCn1cc2ncc(N[C@@H](C)c3cc(NC(=O)c4cnc(N5CCCC5)c(C)c4)ccc3F)nc2n1] | 488.56 | 489.4 |
| 337 | 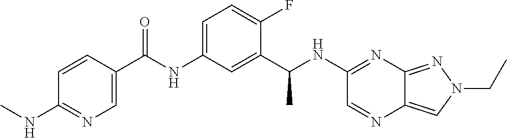 [SMILES: CCn1cc2ncc(N[C@@H](C)c3cc(NC(=O)c4ccc(NC)nc4)ccc3F)nc2n1] | 434.47 | 435.2 |
| 338 | 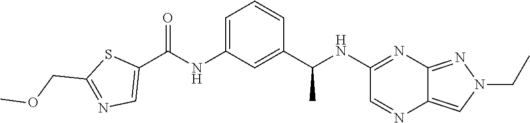 [SMILES: CCn1cc2ncc(N[C@@H](C)c3cccc(NC(=O)c4cnc(COC)s4)c3)nc2n1] | 437.52 | 438.2 |
| 339 | 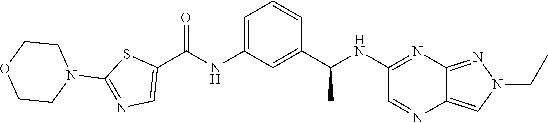 [SMILES: CCn1cc2ncc(N[C@@H](C)c3cccc(NC(=O)c4cnc(N5CCOCC5)s4)c3)nc2n1] | 478.57 | 479.3 |
| 340 | 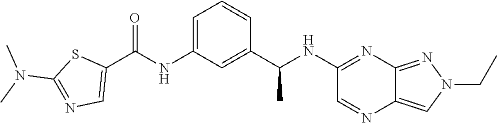 [SMILES: CCn1cc2ncc(N[C@@H](C)c3cccc(NC(=O)c4cnc(N(C)C)s4)c3)nc2n1] | 436.53 | 437.2 |
| 341 | 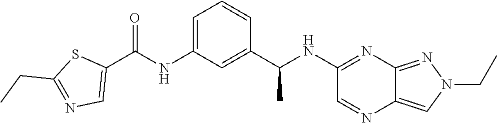 [SMILES: CCc1ncc(C(=O)Nc2cccc([C@H](C)Nc3cnc4cn(CC)nc4n3)c2)s1] | 421.52 | 422.3 |
| 342 | 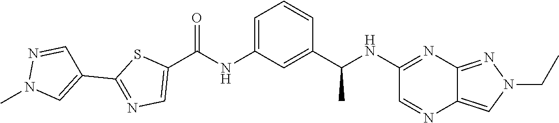 [SMILES: CCn1cc2ncc(N[C@@H](C)c3cccc(NC(=O)c4cnc(-c5cnn(C)c5)s4)c3)nc2n1] | 473.55 | 474.3 |
| 343 | 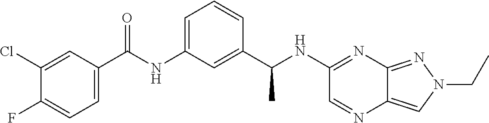 [SMILES: CCn1cc2ncc(N[C@@H](C)c3cccc(NC(=O)c4ccc(F)c(Cl)c4)c3)nc2n1] | 438.88 | 439.8 |
| 344 | 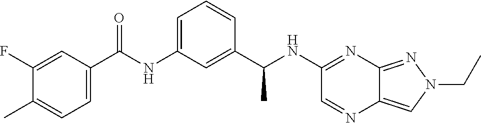 [SMILES: CCn1cc2ncc(N[C@@H](C)c3cccc(NC(=O)c4ccc(C)c(F)c4)c3)nc2n1] | 418.47 | 420.13 |
| 345 | 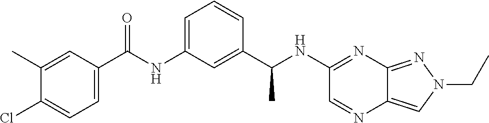 [SMILES: CCn1cc2ncc(N[C@@H](C)c3cccc(NC(=O)c4ccc(Cl)c(C)c4)c3)nc2n1] | 434.92 | 435.7 |
| 346 | 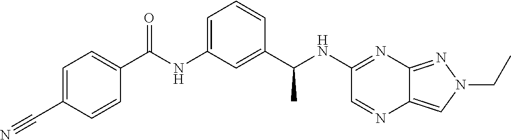 [SMILES: CCn1cc2ncc(N[C@@H](C)c3cccc(NC(=O)c4ccc(C#N)cc4)c3)nc2n1] | 411.46 | 413.36 |
| 347 | 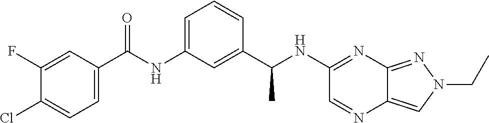 [SMILES: CCn1cc2ncc(N[C@@H](C)c3cccc(NC(=O)c4ccc(Cl)c(F)c4)c3)nc2n1] | 438.88 | 439.4 |
| 348 | 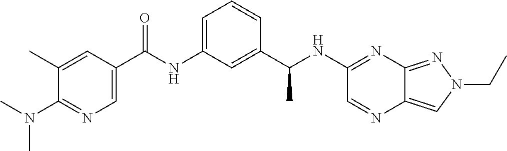 [SMILES: CCn1cc2ncc(N[C@@H](C)c3cccc(NC(=O)c4cnc(N(C)C)c(C)c4)c3)nc2n1] | 444.53 | 445.3 |
| 349 | 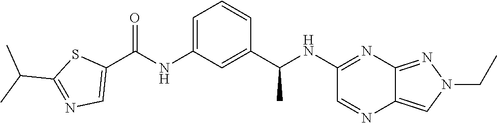 [SMILES: CCn1cc2ncc(N[C@@H](C)c3cccc(NC(=O)c4cnc(C(C)C)s4)c3)nc2n1] | 435.54 | 436.2 |
| 350 | 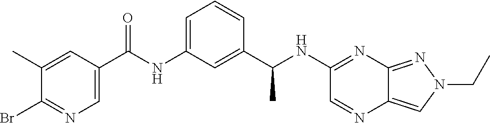 [SMILES: CCn1cc2ncc(N[C@@H](C)c3cccc(NC(=O)c4cnc(Br)c(C)c4)c3)nc2n1] | 480.36 | 480.2 |
| 351 | 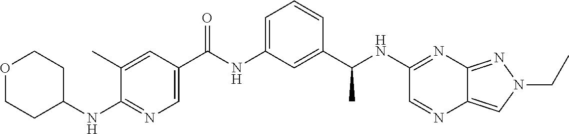 [SMILES: CCn1cc2ncc(N[C@@H](C)c3cccc(NC(=O)c4cnc(NC5CCOCC5)c(C)c4)c3)nc2n1] | 500.59 | 501.3 |
| 352 | 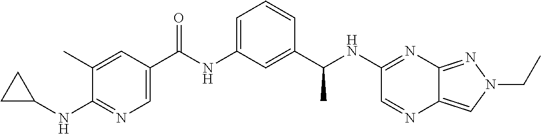 [SMILES: CCn1cc2ncc(N[C@@H](C)c3cccc(NC(=O)c4cnc(NC5CC5)c(C)c4)c3)nc2n1] | 456.54 | 458.07 |
| 353 | 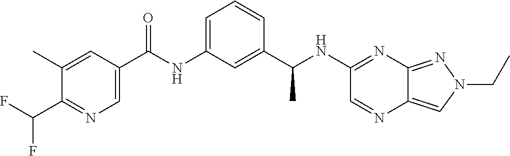 [SMILES: CCn1cc2ncc(N[C@@H](C)c3cccc(NC(=O)c4cnc(C(F)F)c(C)c4)c3)nc2n1] | 451.47 | 453.42 |
| 354 | 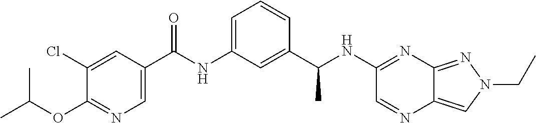 [SMILES: CCn1cc2ncc(N[C@@H](C)c3cccc(NC(=O)c4cnc(OC(C)C)c(Cl)c4)c3)nc2n1] | 479.96 | 480.8 |
| 355 | 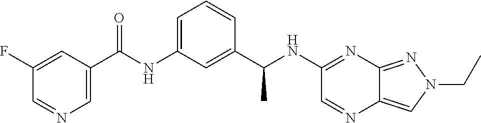 [SMILES: CCn1cc2ncc(N[C@@H](C)c3cccc(NC(=O)c4cncc(F)c4)c3)nc2n1] | 405.43 | 407.1 |
| 356 | 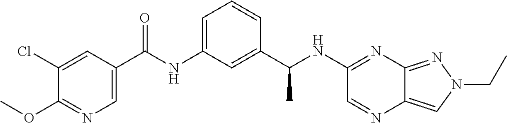 [SMILES: CCn1cc2ncc(N[C@@H](C)c3cccc(NC(=O)c4cnc(OC)c(Cl)c4)c3)nc2n1] | 451.91 | 452.7 |
| 357 | 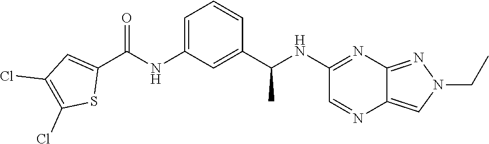 [SMILES: CCn1cc2ncc(N[C@@H](C)c3cccc(NC(=O)c4cc(Cl)c(Cl)s4)c3)nc2n1] | 461.37 | 464.4 |
| 358 | 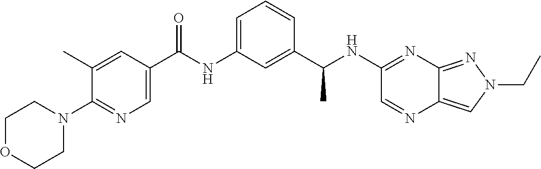 [SMILES: CCn1cc2ncc(N[C@@H](C)c3cccc(NC(=O)c4cnc(N5CCOCC5)c(C)c4)c3)nc2n1] | 486.57 | 487.3 |
| 359 | 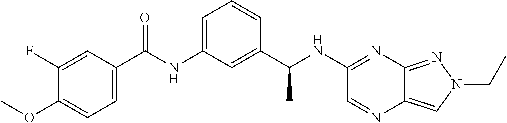 [SMILES: CCn1cc2ncc(N[C@@H](C)c3cccc(NC(=O)c4ccc(OC)c(F)c4)c3)nc2n1] | 434.47 | 436.1 |
| 360 | 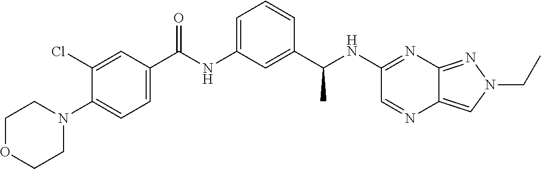 [SMILES: CCn1cc2ncc(N[C@@H](C)c3cccc(NC(=O)c4ccc(N5CCOCC5)c(Cl)c4)c3)nc2n1] | 506 | 508.2 |
| 361 | 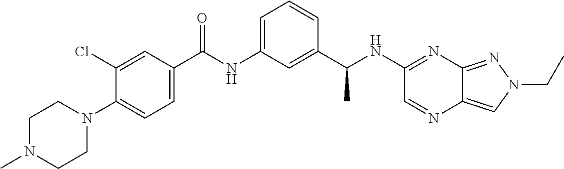 [SMILES: CCn1cc2ncc(N[C@@H](C)c3cccc(NC(=O)c4ccc(N5CCN(C)CC5)c(Cl)c4)c3)nc2n1] | 519.04 | 519.4 |
| 362 | 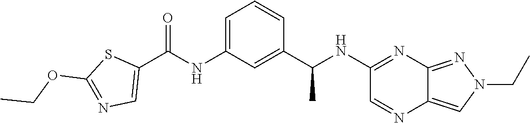 [SMILES: CCOc1ncc(C(=O)Nc2cccc([C@H](C)Nc3cnc4cn(CC)nc4n3)c2)s1] | 437.52 | 438.1 |
| 363 | 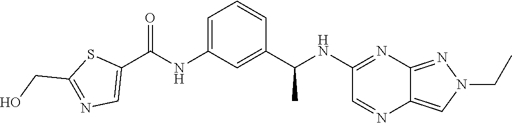 [SMILES: CCn1cc2ncc(N[C@@H](C)c3cccc(NC(=O)c4cnc(CO)s4)c3)nc2n1] | 423.49 | 424.1 |
| 364 | 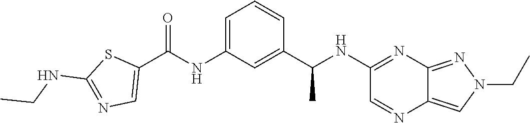 [SMILES: CCNc1ncc(C(=O)Nc2cccc([C@H](C)Nc3cnc4cn(CC)nc4n3)c2)s1] | 436.53 | 437.3 |
| 365 | 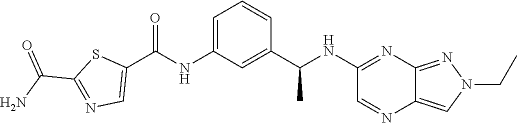 [SMILES: CCn1cc2ncc(N[C@@H](C)c3cccc(NC(=O)c4cnc(C(N)=O)s4)c3)nc2n1] | 436.49 | 437.2 |
| 366 | 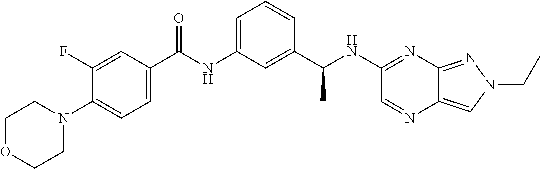 [SMILES: CCn1cc2ncc(N[C@@H](C)c3cccc(NC(=O)c4ccc(N5CCOCC5)c(F)c4)c3)nc2n1] | 489.54 | 490.9 |
| 367 | 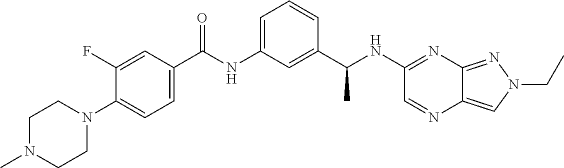 [SMILES: CCn1cc2ncc(N[C@@H](C)c3cccc(NC(=O)c4ccc(N5CCN(C)CC5)c(F)c4)c3)nc2n1] | 502.59 | 503.4 |
| 368 | 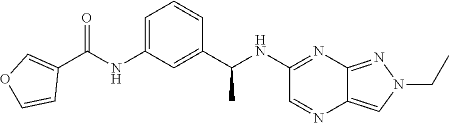 [SMILES: CCn1cc2ncc(N[C@@H](C)c3cccc(NC(=O)c4ccoc4)c3)nc2n1] | 376.41 | 378.1 |
| 369 | 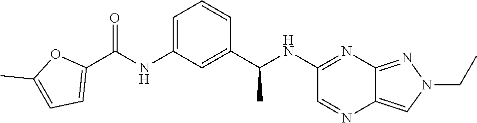 [SMILES: CCn1cc2ncc(N[C@@H](C)c3cccc(NC(=O)c4ccc(C)o4)c3)nc2n1] | 390.44 | 392.2 |
| 370 | 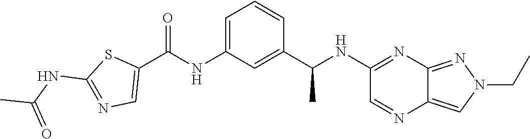 [SMILES: CCn1cc2ncc(N[C@@H](C)c3cccc(NC(=O)c4cnc(NC(C)=O)s4)c3)nc2n1] | 450.52 | 451.1 |
| 371 | 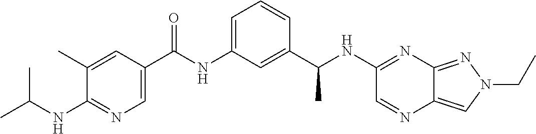 [SMILES: CCn1cc2ncc(N[C@@H](C)c3cccc(NC(=O)c4cnc(NC(C)C)c(C)c4)c3)nc2n1] | 458.56 | 459.3 |
| 372 | 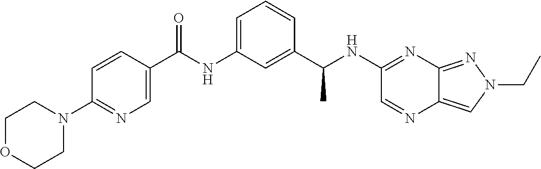 [SMILES: CCn1cc2ncc(N[C@@H](C)c3cccc(NC(=O)c4ccc(N5CCOCC5)nc4)c3)nc2n1] | 472.54 | 473.3 |
| 373 | 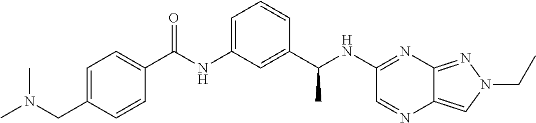 [SMILES: CCn1cc2ncc(N[C@@H](C)c3cccc(NC(=O)c4ccc(CN(C)C)cc4)c3)nc2n1] | 443.54 | 444.4 |
| 374 | 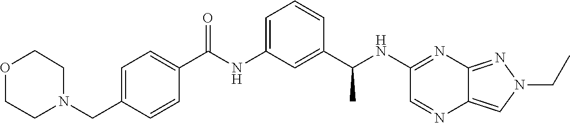 [SMILES: CCn1cc2ncc(N[C@@H](C)c3cccc(NC(=O)c4ccc(CN5CCOCC5)cc4)c3)nc2n1] | 485.58 | 486.4 |
| 375 | 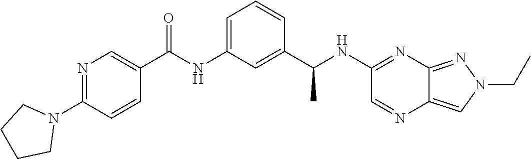 [SMILES: CCn1cc2ncc(N[C@@H](C)c3cccc(NC(=O)c4ccc(N5CCCC5)nc4)c3)nc2n1] | 456.54 | 457.4 |
| 376 | 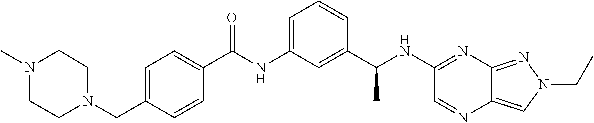 [SMILES: CCn1cc2ncc(N[C@@H](C)c3cccc(NC(=O)c4ccc(CN5CCN(C)CC5)cc4)c3)nc2n1] | 498.62 | 499.4 |
| 377 | 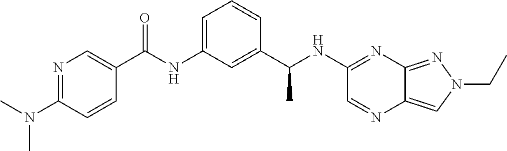 [SMILES: CCn1cc2ncc(N[C@@H](C)c3cccc(NC(=O)c4ccc(N(C)C)nc4)c3)nc2n1] | 430.5 | 431.3 |
| 378 | 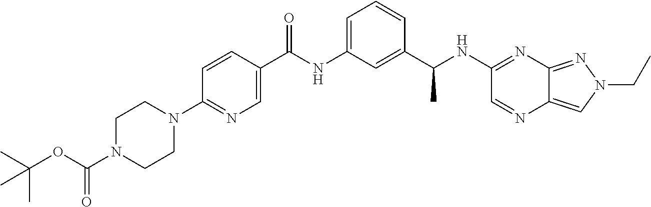 [SMILES: CCn1cc2ncc(N[C@@H](C)c3cccc(NC(=O)c4ccc(N5CCN(C(=O)OC(C)(C)C)CC5)nc4)c3)nc2n1] | 571.67 | 572.4 |
| 379 | 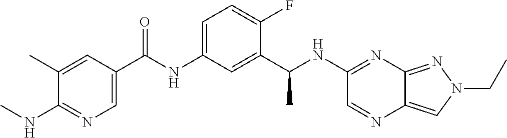 [SMILES: CCn1cc2ncc(N[C@@H](C)c3cc(NC(=O)c4cnc(NC)c(C)c4)ccc3F)nc2n1] | 448.5 | 449.3 |
| 380 | 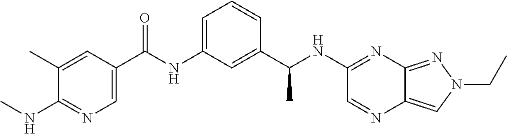 [SMILES: CCn1cc2ncc(N[C@@H](C)c3cccc(NC(=O)c4cnc(NC)c(C)c4)c3)nc2n1] | 430.5 | 431.2 |
| 381 | 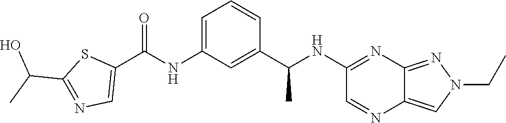 [SMILES: CCn1cc2ncc(N[C@@H](C)c3cccc(NC(=O)c4cnc(C(C)O)s4)c3)nc2n1] | 437.52 | 438.2 |
| 382 | 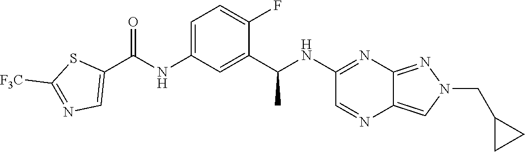 [SMILES: C[C@H](Nc1cnc2cn(CC3CC3)nc2n1)c1cc(NC(=O)c2cnc(C(F)(F)F)s2)ccc1F] | 505.49 | 506.2 |
| 383 | 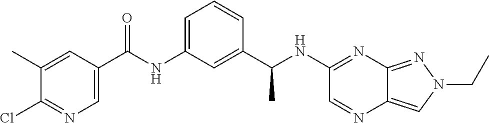 [SMILES: CCn1cc2ncc(N[C@@H](C)c3cccc(NC(=O)c4cnc(Cl)c(C)c4)c3)nc2n1] | 435.91 | 438.2 |
| 384 | 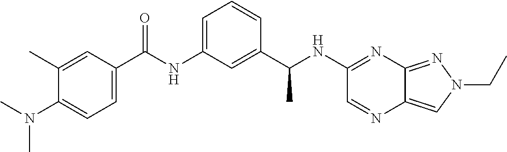 [SMILES: CCn1cc2ncc(N[C@@H](C)c3cccc(NC(=O)c4ccc(N(C)C)c(C)c4)c3)nc2n1] | 443.54 | 444.3 |
| 385 | 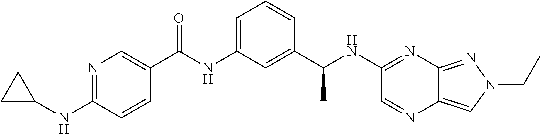 [SMILES: CCn1cc2ncc(N[C@@H](C)c3cccc(NC(=O)c4ccc(NC5CC5)nc4)c3)nc2n1] | 442.52 | 443.3 |
| 386 | 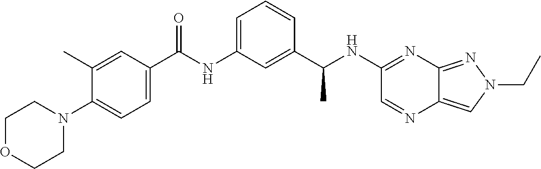 [SMILES: CCn1cc2ncc(N[C@@H](C)c3cccc(NC(=O)c4ccc(N5CCOCC5)c(C)c4)c3)nc2n1] | 485.58 | 486.4 |
| 387 | 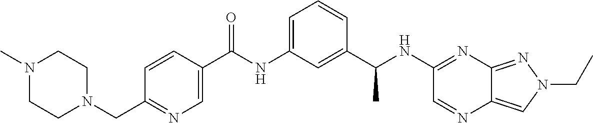 [SMILES: CCn1cc2ncc(N[C@@H](C)c3cccc(NC(=O)c4ccc(CN5CCN(C)CC5)nc4)c3)nc2n1] | 499.61 | 500.4 |
| 388 | 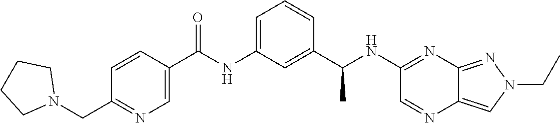 [SMILES: CCn1cc2ncc(N[C@@H](C)c3cccc(NC(=O)c4ccc(CN5CCCC5)nc4)c3)nc2n1] | 470.57 | 471.3 |
| 389 | 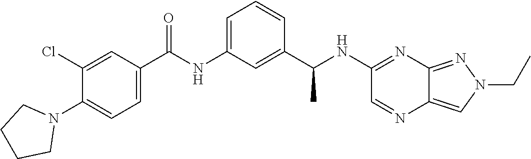 [SMILES: CCn1cc2ncc(N[C@@H](C)c3cccc(NC(=O)c4ccc(N5CCCC5)c(Cl)c4)c3)nc2n1] | 490 | 490.3 |
| 390 | 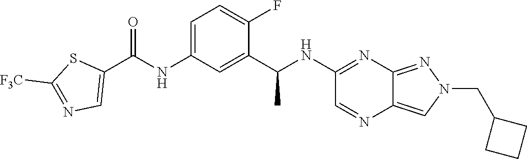 [SMILES: C[C@H](Nc1cnc2cn(CC3CCC3)nc2n1)c1cc(NC(=O)c2cnc(C(F)(F)F)s2)ccc1F] | 519.52 | 520.2 |
| 391 | 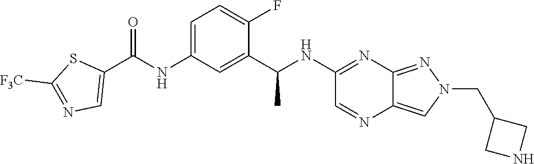 [SMILES: C[C@H](Nc1cnc2cn(CC3CNC3)nc2n1)c1cc(NC(=O)c2cnc(C(F)(F)F)s2)ccc1F] | 520.51 | 521.2 |
| 392 | 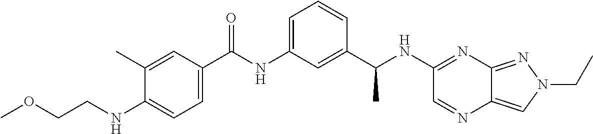 [SMILES: CCn1cc2ncc(N[C@@H](C)c3cccc(NC(=O)c4ccc(NCCOC)c(C)c4)c3)nc2n1] | 474.56 | 475.7 |
| 393 | 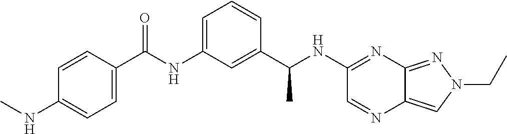 [SMILES: CCn1cc2ncc(N[C@@H](C)c3cccc(NC(=O)c4ccc(NC)cc4)c3)nc2n1] | 416.48 | 417.3 |
| 394 | 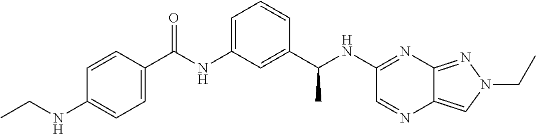 [SMILES: CCNc1ccc(C(=O)Nc2cccc([C@H](C)Nc3cnc4cn(CC)nc4n3)c2)cc1] | 430.5 | 431.3 |
| 395 | 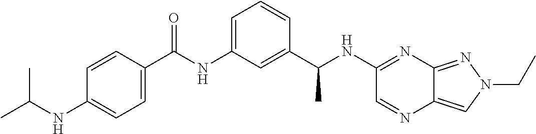 [SMILES: CCn1cc2ncc(N[C@@H](C)c3cccc(NC(=O)c4ccc(NC(C)C)cc4)c3)nc2n1] | 444.53 | 445.7 |
| 396 | 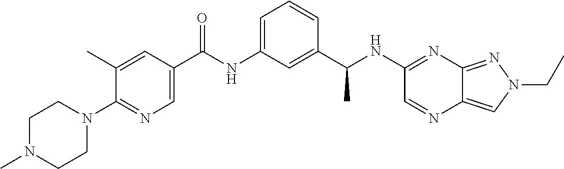 [SMILES: CCn1cc2ncc(N[C@@H](C)c3cccc(NC(=O)c4cnc(N5CCN(C)CC5)c(C)c4)c3)nc2n1] | 499.61 | 500.6 |
| 397 | 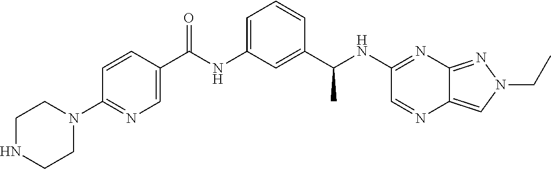 [SMILES: CCn1cc2ncc(N[C@@H](C)c3cccc(NC(=O)c4ccc(N5CCNCC5)nc4)c3)nc2n1] | 471.56 | 472.4 |
| 398 | 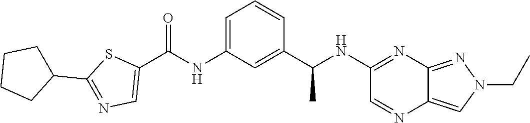 [SMILES: CCn1cc2ncc(N[C@@H](C)c3cccc(NC(=O)c4cnc(C5CCCC5)s4)c3)nc2n1] | 461.58 | 462.3 |
| 399 | 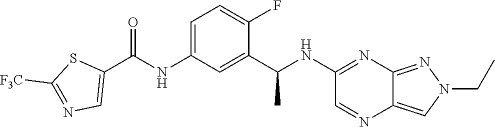 [SMILES: CCn1cc2ncc(N[C@@H](C)c3cc(NC(=O)c4cnc(C(F)(F)F)s4)ccc3F)nc2n1] | 491.46 | 492.2 |
| 400 | 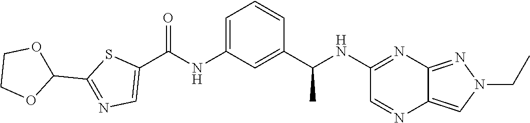 [SMILES: CCn1cc2ncc(N[C@@H](C)c3cccc(NC(=O)c4cnc(C5OCCO5)s4)c3)nc2n1] | 465.53 | 467.4 |
| 401 | 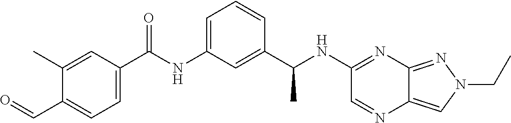 [SMILES: CCn1cc2ncc(N[C@@H](C)c3cccc(NC(=O)c4ccc(C=O)c(C)c4)c3)nc2n1] | 428.49 | 429 |
| 402 | 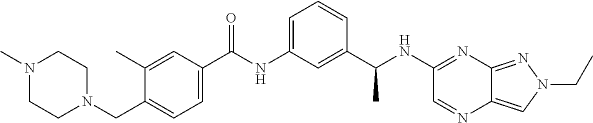 [SMILES: CCn1cc2ncc(N[C@@H](C)c3cccc(NC(=O)c4ccc(CN5CCN(C)CC5)c(C)c4)c3)nc2n1] | 512.65 | 513.3 |
| 403 | 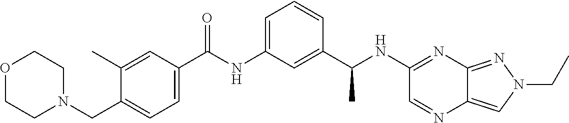 [SMILES: CCn1cc2ncc(N[C@@H](C)c3cccc(NC(=O)c4ccc(CN5CCOCC5)c(C)c4)c3)nc2n1] | 499.61 | 500.1 |
| 404 | 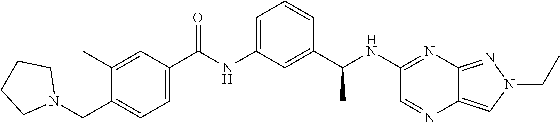 [SMILES: CCn1cc2ncc(N[C@@H](C)c3cccc(NC(=O)c4ccc(CN5CCCC5)c(C)c4)c3)nc2n1] | 483.61 | 484.6 |
| 405 | 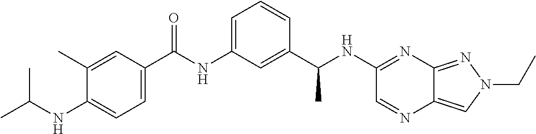 [SMILES: CCn1cc2ncc(N[C@@H](C)c3cccc(NC(=O)c4ccc(NC(C)C)c(C)c4)c3)nc2n1] | 457.57 | 458.2 |
| 406 | 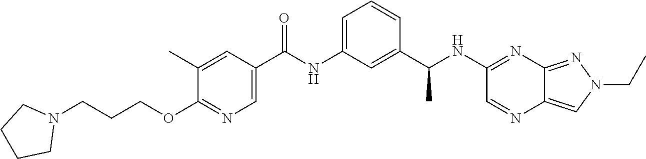 [SMILES: CCn1cc2ncc(N[C@@H](C)c3cccc(NC(=O)c4cnc(OCCCN5CCCC5)c(C)c4)c3)nc2n1] | 528.65 | 529.8 |
| 407 | 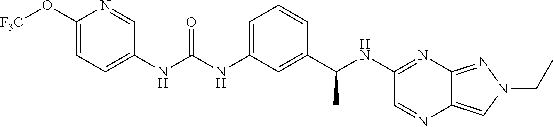 [SMILES: CCn1cc2ncc(N[C@@H](C)c3cccc(NC(=O)Nc4ccc(OC(F)(F)F)nc4)c3)nc2n1] | 486.45 | 487.2 |
| 408 | 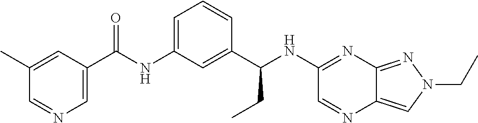 [SMILES: CC[C@H](Nc1cnc2cn(CC)nc2n1)c1cccc(NC(=O)c2cncc(C)c2)c1] | 415.49 | 416.3 |
| 409 | 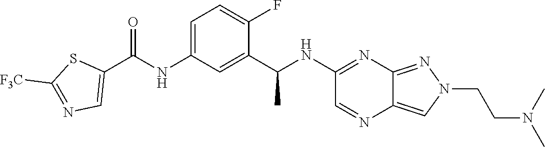 [SMILES: C[C@H](Nc1cnc2cn(CCN(C)C)nc2n1)c1cc(NC(=O)c2cnc(C(F)(F)F)s2)ccc1F] | 522.52 | 523.2 |
| 410 | 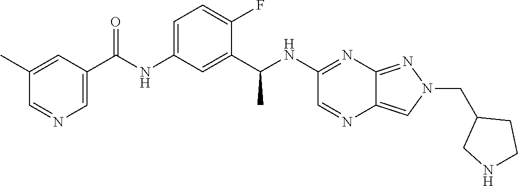 [SMILES: Cc1cncc(C(=O)Nc2ccc(F)c([C@H](C)Nc3cnc4cn(CC5CCNC5)nc4n3)c2)c1] | 474.53 | 475.3 |
| 411 | 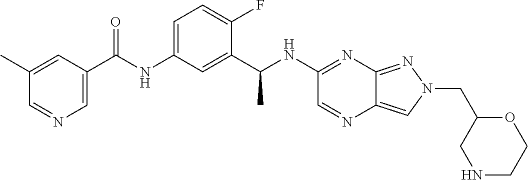 [SMILES: Cc1cncc(C(=O)Nc2ccc(F)c([C@H](C)Nc3cnc4cn(CC5CNCCO5)nc4n3)c2)c1] | 490.53 | 491.2 |
| 412 | 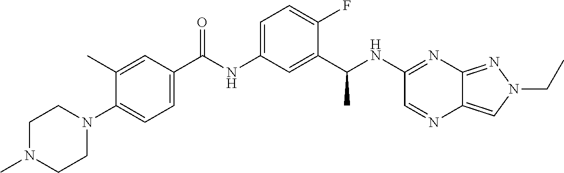 [SMILES: CCn1cc2ncc(N[C@@H](C)c3cc(NC(=O)c4ccc(N5CCN(C)CC5)c(C)c4)ccc3F)nc2n1] | 516.61 | 517.25 |
| 413 | 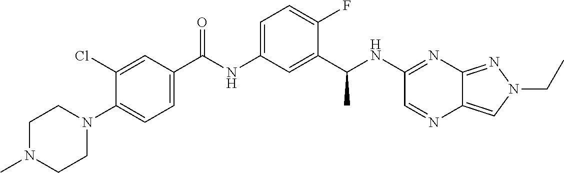 [SMILES: CCn1cc2ncc(N[C@@H](C)c3cc(NC(=O)c4ccc(N5CCN(C)CC5)c(Cl)c4)ccc3F)nc2n1] | 537.03 | 537.1 |
| 414 | 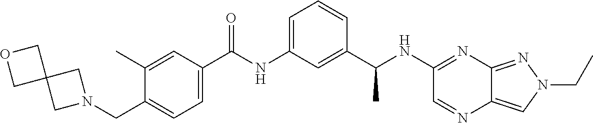 [SMILES: CCn1cc2ncc(N[C@@H](C)c3cccc(NC(=O)c4ccc(CN5CC6(COC6)C5)c(C)c4)c3)nc2n1] | 511.62 | 512.2 |
| 415 | 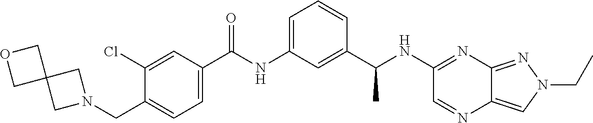 [SMILES: CCn1cc2ncc(N[C@@H](C)c3cccc(NC(=O)c4ccc(CN5CC6(COC6)C5)c(Cl)c4)c3)nc2n1] | 532.04 | 532.2 |
| 416 | 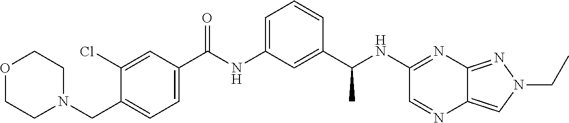 [SMILES: CCn1cc2ncc(N[C@@H](C)c3cccc(NC(=O)c4ccc(CN5CCOCC5)c(Cl)c4)c3)nc2n1] | 520.02 | 520.2 |
| 417 | 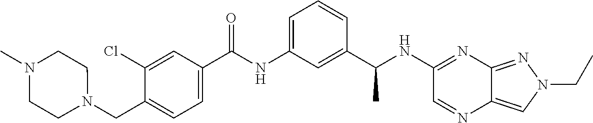 [SMILES: CCn1cc2ncc(N[C@@H](C)c3cccc(NC(=O)c4ccc(CN5CCN(C)CC5)c(Cl)c4)c3)nc2n1] | 533.07 | 533.4 |
| 418 | 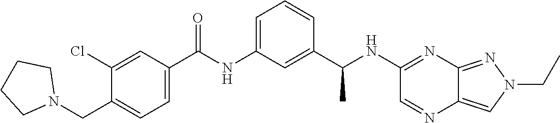 [SMILES: CCn1cc2ncc(N[C@@H](C)c3cccc(NC(=O)c4ccc(CN5CCCC5)c(Cl)c4)c3)nc2n1] | 504.03 | 504.2 |
| 419 | 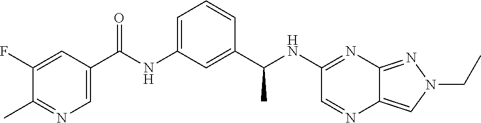 [SMILES: CCn1cc2ncc(N[C@@H](C)c3cccc(NC(=O)c4cnc(C)c(F)c4)c3)nc2n1] | 419.45 | 420.2 |
| 420 | 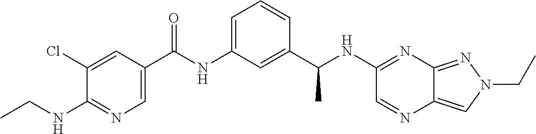 [SMILES: CCNc1ncc(C(=O)Nc2cccc([C@H](C)Nc3cnc4cn(CC)nc4n3)c2)cc1Cl] | 464.95 | 465.1 |
| 421 | 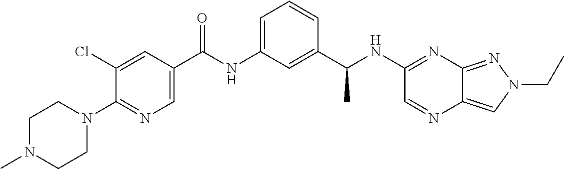 [SMILES: CCn1cc2ncc(N[C@@H](C)c3cccc(NC(=O)c4cnc(N5CCN(C)CC5)c(Cl)c4)c3)nc2n1] | 520.03 | 520.2 |
| 422 | 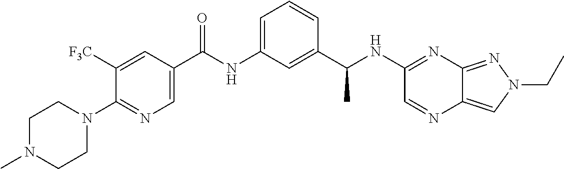 [SMILES: CCn1cc2ncc(N[C@@H](C)c3cccc(NC(=O)c4cnc(N5CCN(C)CC5)c(C(F)(F)F)c4)c3)nc2n1] | 553.58 | 554.3 |
| 423 | 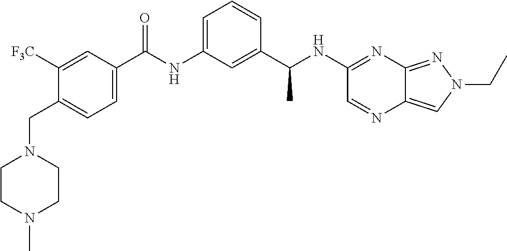 [SMILES: CCn1cc2ncc(N[C@@H](C)c3cccc(NC(=O)c4ccc(CN5CCN(C)CC5)c(C(F)(F)F)c4)c3)nc2n1] | 566.62 | 567.3 |
| 424 | 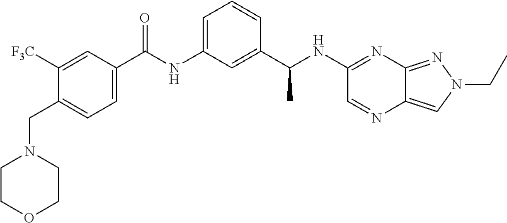 [SMILES: CCn1cc2ncc(N[C@@H](C)c3cccc(NC(=O)c4ccc(CN5CCOCC5)c(C(F)(F)F)c4)c3)nc2n1] | 553.58 | 554.2 |
| 425 | 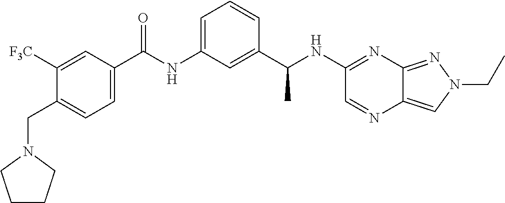 [SMILES: CCn1cc2ncc(N[C@@H](C)c3cccc(NC(=O)c4ccc(CN5CCCC5)c(C(F)(F)F)c4)c3)nc2n1] | 537.58 | 538.3 |
| 426 | 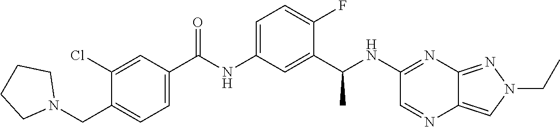 [SMILES: CCn1cc2ncc(N[C@@H](C)c3cc(NC(=O)c4ccc(CN5CCCC5)c(Cl)c4)ccc3F)nc2n1] | 522.02 | 522.35 |
| 427 | 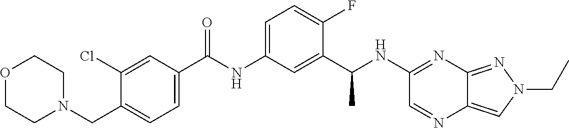 [SMILES: CCn1cc2ncc(N[C@@H](C)c3cc(NC(=O)c4ccc(CN5CCOCC5)c(Cl)c4)ccc3F)nc2n1] | 538.02 | 538.2 |
| 428 | 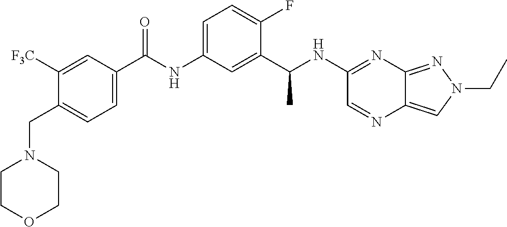 [SMILES: CCn1cc2ncc(N[C@@H](C)c3cc(NC(=O)c4ccc(CN5CCOCC5)c(C(F)(F)F)c4)ccc3F)nc2n1] | 571.57 | 572.3 |
| 429 | 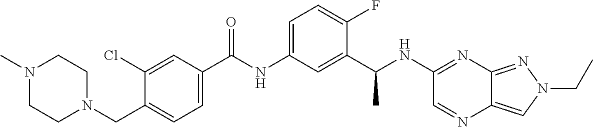 [SMILES: CCn1cc2ncc(N[C@@H](C)c3cc(NC(=O)c4ccc(CN5CCN(C)CC5)c(Cl)c4)ccc3F)nc2n1] | 551.06 | 551.4 |
| 430 | 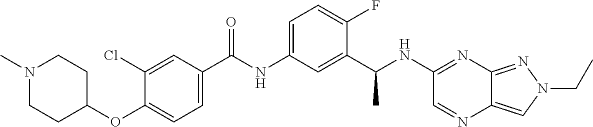 [SMILES: CCn1cc2ncc(N[C@@H](C)c3cc(NC(=O)c4ccc(OC5CCN(C)CC5)c(Cl)c4)ccc3F)nc2n1] | 552.04 | 552.3 |
| 431 | 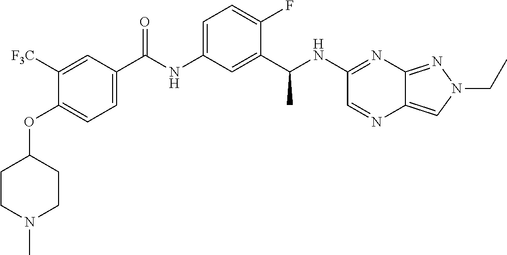 [SMILES: CCn1cc2ncc(N[C@@H](C)c3cc(NC(=O)c4ccc(OC5CCN(C)CC5)c(C(F)(F)F)c4)ccc3F)nc2n1] | 585.59 | 586.2 |
| 432 | 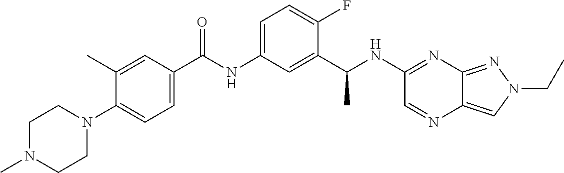 [SMILES: CCn1cc2ncc(N[C@@H](C)c3cc(NC(=O)c4ccc(N5CCN(C)CC5)c(C)c4)ccc3F)nc2n1] | 517.6 | 518.2 |
| 433 | 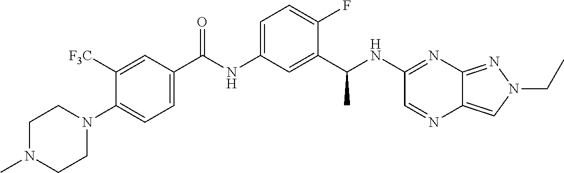 [SMILES: CCn1cc2ncc(N[C@@H](C)c3cc(NC(=O)c4ccc(N5CCN(C)CC5)c(C(F)(F)F)c4)ccc3F)nc2n1] | 570.58 | 571.2 |
| 434 | 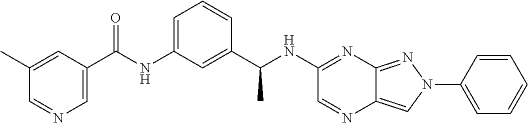 [SMILES: Cc1cncc(C(=O)Nc2cccc([C@H](C)Nc3cnc4cn(-c5ccccc5)nc4n3)c2)c1] | 449.51 | 450.1 |
| 435 | 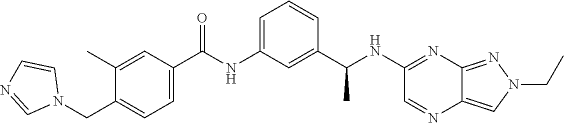 [SMILES: CCn1cc2ncc(N[C@@H](C)c3cccc(NC(=O)c4ccc(Cn5ccnc5)c(C)c4)c3)nc2n1] | 480.56 | 481.1 |
| 436 | 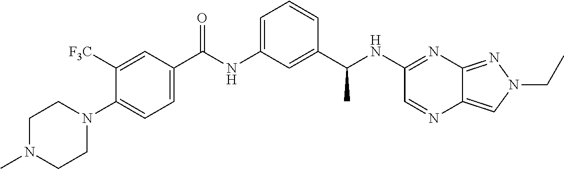 [SMILES: CCn1cc2ncc(N[C@@H](C)c3cccc(NC(=O)c4ccc(N5CCN(C)CC5)c(C(F)(F)F)c4)c3)nc2n1] | 552.59 | 553.2 |
| 437 | 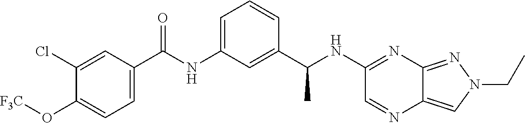 [SMILES: CCn1cc2ncc(N[C@@H](C)c3cccc(NC(=O)c4ccc(OC(F)(F)F)c(Cl)c4)c3)nc2n1] | 504.89 | 505.7 |
| 438 | 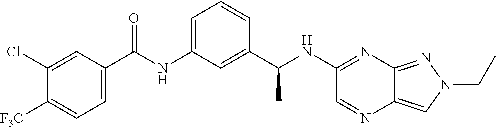 [SMILES: CCn1cc2ncc(N[C@@H](C)c3cccc(NC(=O)c4ccc(C(F)(F)F)c(Cl)c4)c3)nc2n1] | 488.89 | 489.6 |
| 439 | 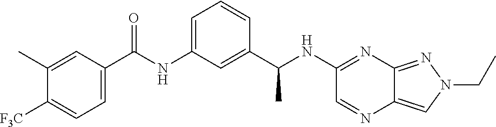 [SMILES: CCn1cc2ncc(N[C@@H](C)c3cccc(NC(=O)c4ccc(C(F)(F)F)c(C)c4)c3)nc2n1] | 468.47 | 470 |
| 440 | 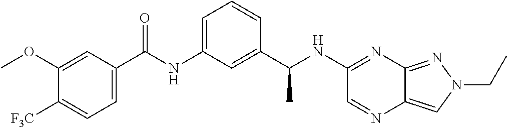 [SMILES: CCn1cc2ncc(N[C@@H](C)c3cccc(NC(=O)c4ccc(C(F)(F)F)c(OC)c4)c3)nc2n1] | 484.47 | 485.9 |
| 441 | 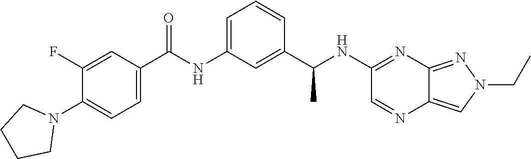 [SMILES: CCn1cc2ncc(N[C@@H](C)c3cccc(NC(=O)c4ccc(N5CCCC5)c(F)c4)c3)nc2n1] | 473.54 | 475 |
| 442 | 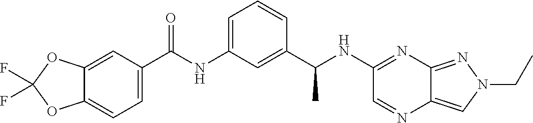 [SMILES: CCn1cc2ncc(N[C@@H](C)c3cccc(NC(=O)c4ccc5c(c4)OC(F)(F)O5)c3)nc2n1] | 466.44 | 467.5 |
| 443 | 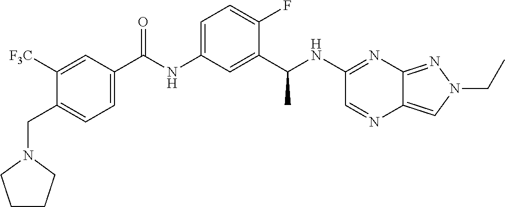 [SMILES: CCn1cc2ncc(N[C@@H](C)c3cc(NC(=O)c4ccc(CN5CCCC5)c(C(F)(F)F)c4)ccc3F)nc2n1] | 555.57 | 556.3 |
| 444 | 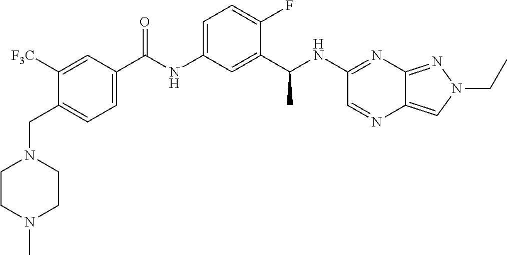 [SMILES: CCn1cc2ncc(N[C@@H](C)c3cc(NC(=O)c4ccc(CN5CCN(C)CC5)c(C(F)(F)F)c4)ccc3F)nc2n1] | 584.61 | 585.3 |
| 445 | 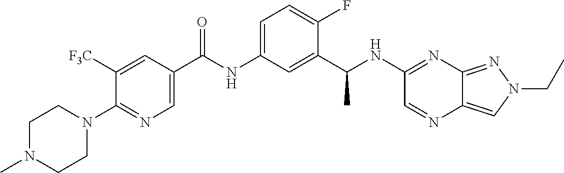 [SMILES: CCn1cc2ncc(N[C@@H](C)c3cc(NC(=O)c4cnc(N5CCN(C)CC5)c(C(F)(F)F)c4)ccc3F)nc2n1] | 571.57 | 572.3 |
| 446 | 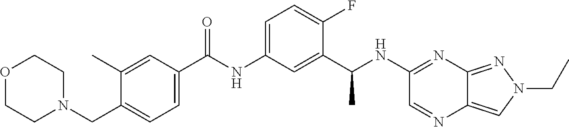 [SMILES: CCn1cc2ncc(N[C@@H](C)c3cc(NC(=O)c4ccc(CN5CCOCC5)c(C)c4)ccc3F)nc2n1] | 517.6 | 518.3 |
| 447 | 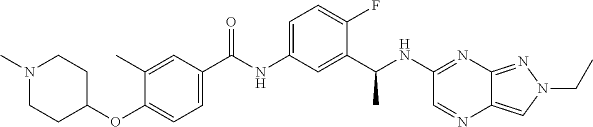 [SMILES: CCn1cc2ncc(N[C@@H](C)c3cc(NC(=O)c4ccc(OC5CCN(C)CC5)c(C)c4)ccc3F)nc2n1] | 531.62 | 532.4 |
| 448 | 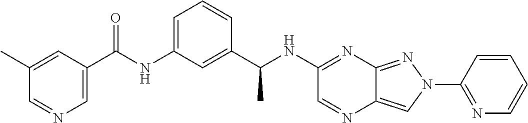 [SMILES: Cc1cncc(C(=O)Nc2cccc([C@H](C)Nc3cnc4cn(-c5ccccn5)nc4n3)c2)c1] | 450.49 | 451.5 |
| 449 | 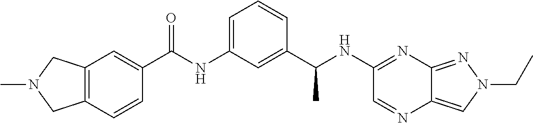 [SMILES: CCn1cc2ncc(N[C@@H](C)c3cccc(NC(=O)c4ccc5c(c4)CN(C)C5)c3)nc2n1] | 441.53 | 442.3 |
| 450 | 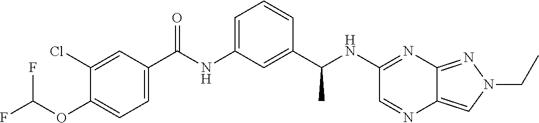 [SMILES: CCn1cc2ncc(N[C@@H](C)c3cccc(NC(=O)c4ccc(OC(F)F)c(Cl)c4)c3)nc2n1] | 486.9 | 487.1 |
| 451 | 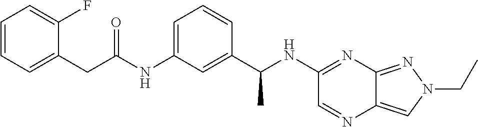 [SMILES: CCn1cc2ncc(N[C@@H](C)c3cccc(NC(=O)Cc4ccccc4F)c3)nc2n1] | 418.47 | 419.1 |
| 452 | 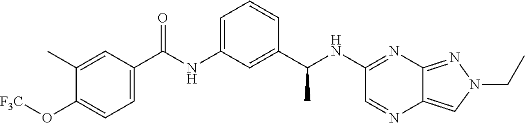 [SMILES: CCn1cc2ncc(N[C@@H](C)c3cccc(NC(=O)c4ccc(OC(F)(F)F)c(C)c4)c3)nc2n1] | 484.47 | 485.6 |
| 453 | 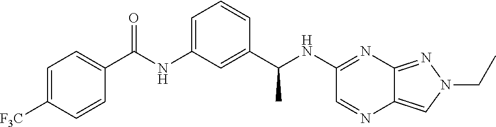 [SMILES: CCn1cc2ncc(N[C@@H](C)c3cccc(NC(=O)c4ccc(C(F)(F)F)cc4)c3)nc2n1] | 454.45 | 455.6 |
| 454 | 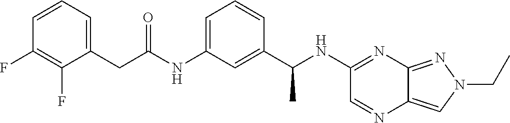 [SMILES: CCn1cc2ncc(N[C@@H](C)c3cccc(NC(=O)Cc4cccc(F)c4F)c3)nc2n1] | 436.46 | 437.5 |
| 455 | 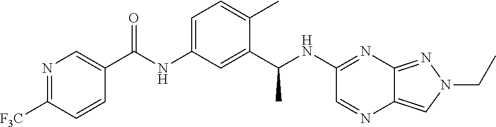 [SMILES: CCn1cc2ncc(N[C@@H](C)c3cc(NC(=O)c4ccc(C(F)(F)F)nc4)ccc3C)nc2n1] | 469.46 | 470.15 |
| 456 | 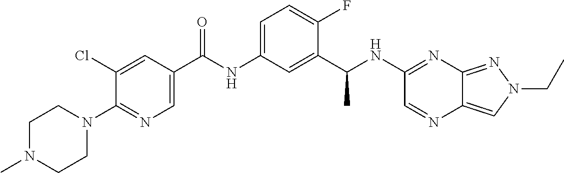 [SMILES: CCn1cc2ncc(N[C@@H](C)c3cc(NC(=O)c4cnc(N5CCN(C)CC5)c(Cl)c4)ccc3F)nc2n1] | 538.02 | 538.1 |
| 457 | 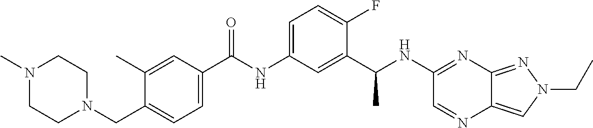 [SMILES: CCn1cc2ncc(N[C@@H](C)c3cc(NC(=O)c4ccc(CN5CCN(C)CC5)c(C)c4)ccc3F)nc2n1] | 530.64 | 531.2 |
| 458 | 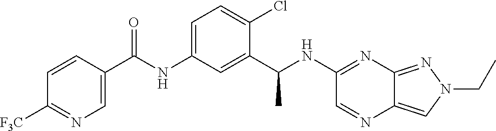 [SMILES: CCn1cc2ncc(N[C@@H](C)c3cc(NC(=O)c4ccc(C(F)(F)F)nc4)ccc3Cl)nc2n1] | 489.88 | 490.2 |
| 459 | 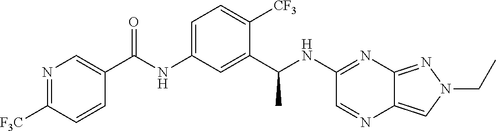 [SMILES: CCn1cc2ncc(N[C@@H](C)c3cc(NC(=O)c4ccc(C(F)(F)F)nc4)ccc3C(F)(F)F)nc2n1] | 523.43 | 524.1 |
| 460 | 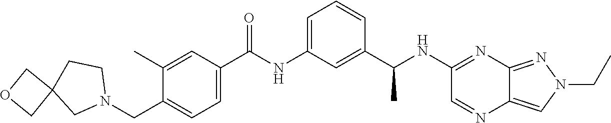 [SMILES: CCn1cc2ncc(N[C@@H](C)c3cccc(NC(=O)c4ccc(CN5CCC6(COC6)C5)c(C)c4)c3)nc2n1] | 525.64 | 526.1 |
| 461 | 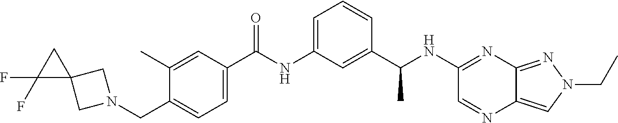 [SMILES: CCn1cc2ncc(N[C@@H](C)c3cccc(NC(=O)c4ccc(CN5CC6(C5)CC6(F)F)c(C)c4)c3)nc2n1] | 531.6 | 532.2 |
| 462 | 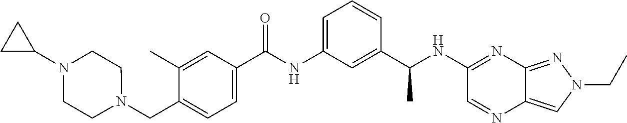 [SMILES: CCn1cc2ncc(N[C@@H](C)c3cccc(NC(=O)c4ccc(CN5CCN(C6CC6)CC5)c(C)c4)c3)nc2n1] | 538.68 | 538.4 |
| 463 | 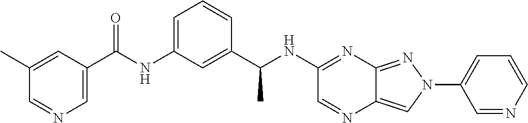 [SMILES: Cc1cncc(C(=O)Nc2cccc([C@H](C)Nc3cnc4cn(-c5cccnc5)nc4n3)c2)c1] | 450.49 | 451.3 |
| 464 | 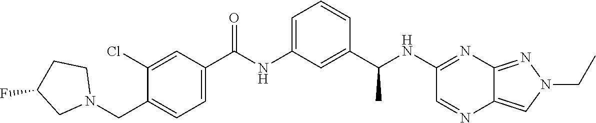 [SMILES: CCn1cc2ncc(N[C@@H](C)c3cccc(NC(=O)c4ccc(CN5CC[C@@H](F)C5)c(Cl)c4)c3)nc2n1] | 522.02 | 522.3 |
| 465 | 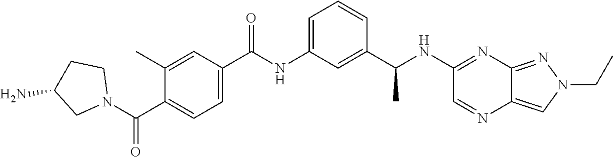 [SMILES: CCn1cc2ncc(N[C@@H](C)c3cccc(NC(=O)c4ccc(C(=O)N5CC[C@@H](N)C5)c(C)c4)c3)nc2n1] | 512.61 | 513.2 |
| 466 | 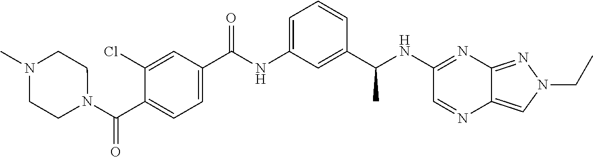 [SMILES: CCn1cc2ncc(N[C@@H](C)c3cccc(NC(=O)c4ccc(C(=O)N5CCN(C)CC5)c(Cl)c4)c3)nc2n1] | 547.05 | 548.1 |
| 467 | 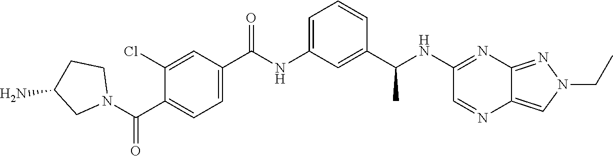 [SMILES: CCn1cc2ncc(N[C@@H](C)c3cccc(NC(=O)c4ccc(C(=O)N5CC[C@@H](N)C5)c(Cl)c4)c3)nc2n1] | 533.02 | 533.1 |
| 468 | 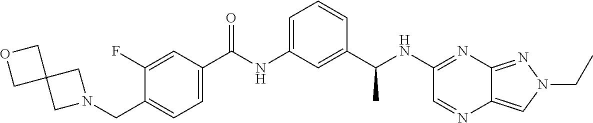 [SMILES: CCn1cc2ncc(N[C@@H](C)c3cccc(NC(=O)c4ccc(CN5CC6(COC6)C5)c(F)c4)c3)nc2n1] | 515.58 | 516.1 |
| 469 | 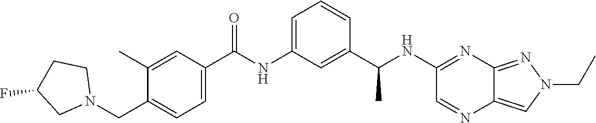 [SMILES: CCn1cc2ncc(N[C@@H](C)c3cccc(NC(=O)c4ccc(CN5CC[C@@H](F)C5)c(C)c4)c3)nc2n1] | 501.6 | 503.2 |
| 470 | 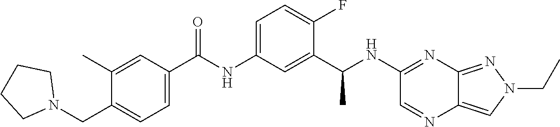 [SMILES: CCn1cc2ncc(N[C@@H](C)c3cc(NC(=O)c4ccc(CN5CCCC5)c(C)c4)ccc3F)nc2n1] | 501.6 | 502.1 |
| 471 | 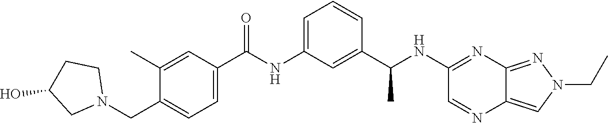 [SMILES: CCn1cc2ncc(N[C@@H](C)c3cccc(NC(=O)c4ccc(CN5CC[C@@H](O)C5)c(C)c4)c3)nc2n1] | 499.61 | 500.4 |
| 472 | 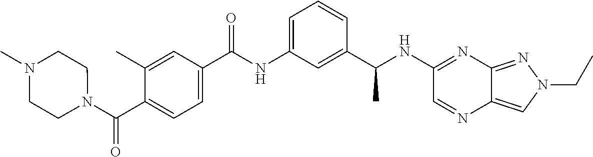 [SMILES: CCn1cc2ncc(N[C@@H](C)c3cccc(NC(=O)c4ccc(C(=O)N5CCN(C)CC5)c(C)c4)c3)nc2n1] | 526.63 | 526.3 |
| 473 | 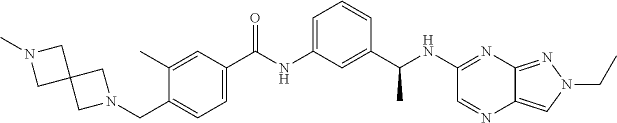 [SMILES: CCn1cc2ncc(N[C@@H](C)c3cccc(NC(=O)c4ccc(CN5CC6(CN(C)C6)C5)c(C)c4)c3)nc2n1] | 524.66 | 526.3 |
| 474 | 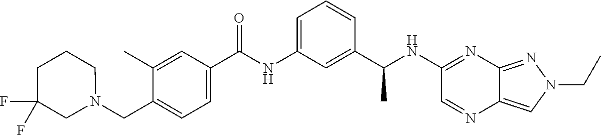 [SMILES: CCn1cc2ncc(N[C@@H](C)c3cccc(NC(=O)c4ccc(CN5CCCC(F)(F)C5)c(C)c4)c3)nc2n1] | 533.61 | 534.5 |
| 475 | 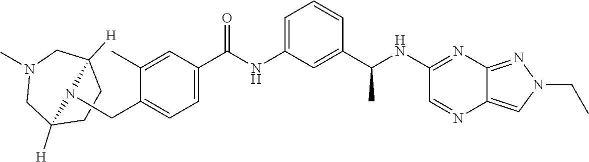 [SMILES: CCn1cc2ncc(N[C@@H](C)c3cccc(NC(=O)c4ccc(CN5[C@@H]6CC[C@H]5CN(C)C6)c(C)c4)c3)nc2n1] | 538.68 | 539.5 |
| 476 | 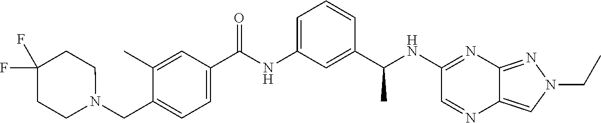 [SMILES: CCn1cc2ncc(N[C@@H](C)c3cccc(NC(=O)c4ccc(CN5CCC(F)(F)CC5)c(C)c4)c3)nc2n1] | 533.61 | 534.5 |
| 477 | 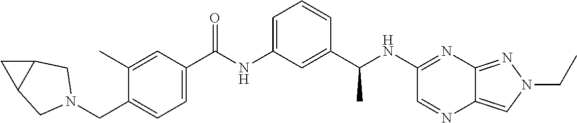 [SMILES: CCn1cc2ncc(N[C@@H](C)c3cccc(NC(=O)c4ccc(CN5CC6CC6C5)c(C)c4)c3)nc2n1] | 495.62 | 496.5 |
| 478 | 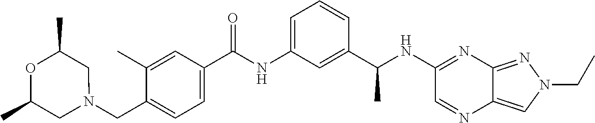 [SMILES: CCn1cc2ncc(N[C@@H](C)c3cccc(NC(=O)c4ccc(CN5C[C@@H](C)O[C@@H](C)C5)c(C)c4)c3)nc2n1] | 527.66 | 529.2 |
| 479 | 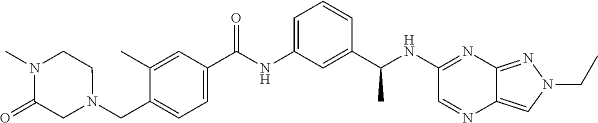 [SMILES: CCn1cc2ncc(N[C@@H](C)c3cccc(NC(=O)c4ccc(CN5CCN(C)C(=O)C5)c(C)c4)c3)nc2n1] | 526.63 | 527.5 |
| 480 | 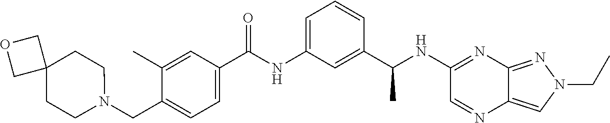 [SMILES: CCn1cc2ncc(N[C@@H](C)c3cccc(NC(=O)c4ccc(CN5CCC6(CC5)COC6)c(C)c4)c3)nc2n1] | 539.67 | 540.3 |
| 481 | 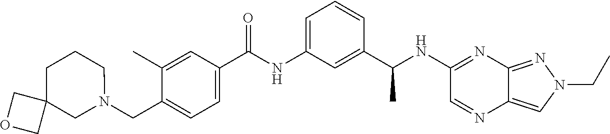 [SMILES: CCn1cc2ncc(N[C@@H](C)c3cccc(NC(=O)c4ccc(CN5CCCC6(COC6)C5)c(C)c4)c3)nc2n1] | 539.67 | 540.5 |
| 482 | 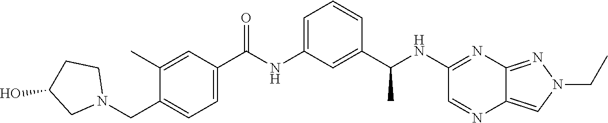 [SMILES: CCn1cc2ncc(N[C@@H](C)c3cccc(NC(=O)c4ccc(CN5CC[C@@H](O)C5)c(C)c4)c3)nc2n1] | 499.61 | 550.15 |
| 483 | 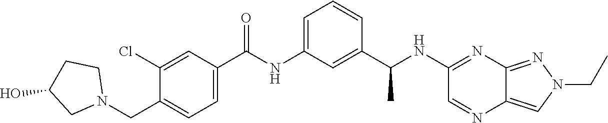 [SMILES: CCn1cc2ncc(N[C@@H](C)c3cccc(NC(=O)c4ccc(CN5CC[C@@H](O)C5)c(Cl)c4)c3)nc2n1] | 520.02 | 520.3 |
| 484 | 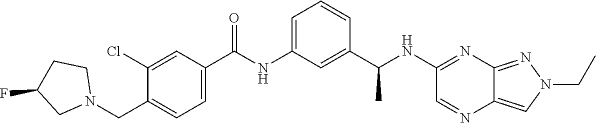 [SMILES: CCn1cc2ncc(N[C@@H](C)c3cccc(NC(=O)c4ccc(CN5CC[C@H](F)C5)c(Cl)c4)c3)nc2n1] | 522.02 | 522.1 |
| 485 | 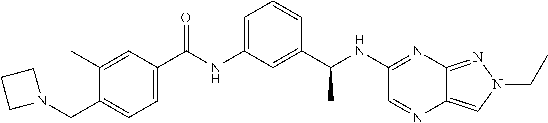 [SMILES: CCn1cc2ncc(N[C@@H](C)c3cccc(NC(=O)c4ccc(CN5CCC5)c(C)c4)c3)nc2n1] | 469.58 | 470.1 |
| 486 | 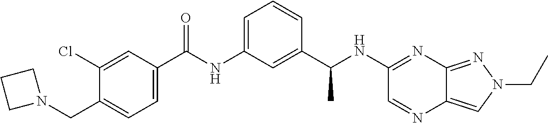 [SMILES: CCn1cc2ncc(N[C@@H](C)c3cccc(NC(=O)c4ccc(CN5CCC5)c(Cl)c4)c3)nc2n1] | 490 | 490.3 |
| 487 | 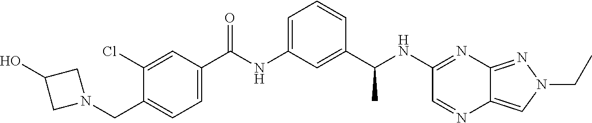 [SMILES: CCn1cc2ncc(N[C@@H](C)c3cccc(NC(=O)c4ccc(CN5CC(O)C5)c(Cl)c4)c3)nc2n1] | 506 | 506 |
| 488 | 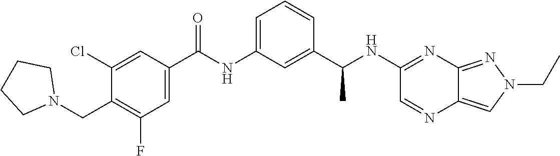 [SMILES: CCn1cc2ncc(N[C@@H](C)c3cccc(NC(=O)c4cc(F)c(CN5CCCC5)c(Cl)c4)c3)nc2n1] | 522.02 | 522.1 |
| 489 | 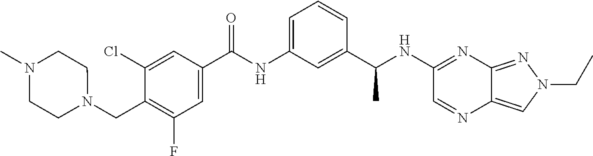 [SMILES: CCn1cc2ncc(N[C@@H](C)c3cccc(NC(=O)c4cc(F)c(CN5CCN(C)CC5)c(Cl)c4)c3)nc2n1] | 551.06 | 551.1 |
| 490 | 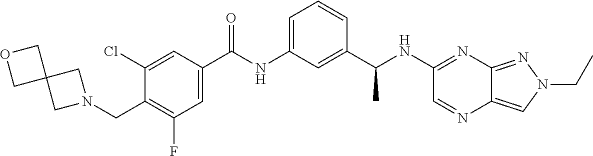 [SMILES: CCn1cc2ncc(N[C@@H](C)c3cccc(NC(=O)c4cc(F)c(CN5CC6(COC6)C5)c(Cl)c4)c3)nc2n1] | 550.03 | 550.15 |
| 491 | 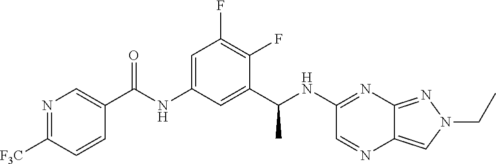 [SMILES: CCn1cc2ncc(N[C@@H](C)c3cc(NC(=O)c4ccc(C(F)(F)F)nc4)cc(F)c3F)nc2n1] | 491.42 | 492.2 |
| 492 | 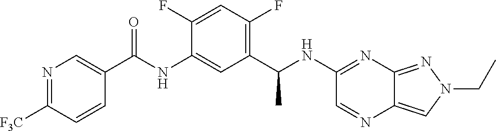 [SMILES: CCn1cc2ncc(N[C@@H](C)c3cc(NC(=O)c4ccc(C(F)(F)F)nc4)c(F)cc3F)nc2n1] | 491.42 | 492.1 |
| 493 | 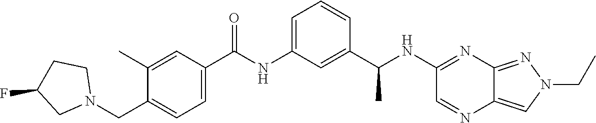 [SMILES: CCn1cc2ncc(N[C@@H](C)c3cccc(NC(=O)c4ccc(CN5CC[C@H](F)C5)c(C)c4)c3)nc2n1] | 501.6 | 502.2 |
| 494 | 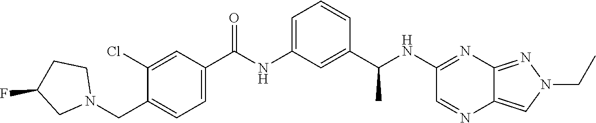 [SMILES: CCn1cc2ncc(N[C@@H](C)c3cccc(NC(=O)c4ccc(CN5CC[C@H](F)C5)c(Cl)c4)c3)nc2n1] | 520.02 | 520.1 |
| 495 | 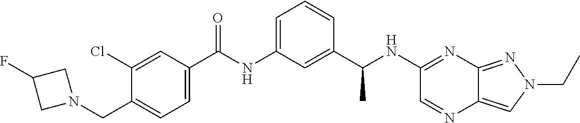 [SMILES: CCn1cc2ncc(N[C@@H](C)c3cccc(NC(=O)c4ccc(CN5CC(F)C5)c(Cl)c4)c3)nc2n1] | 507.99 | 508.1 |
| 496 | 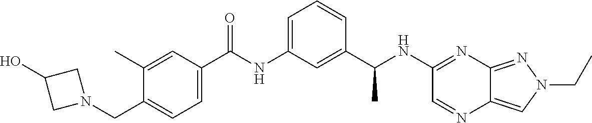 [SMILES: CCn1cc2ncc(N[C@@H](C)c3cccc(NC(=O)c4ccc(CN5CC(O)C5)c(C)c4)c3)nc2n1] | 485.58 | 486.1 |
| 497 | 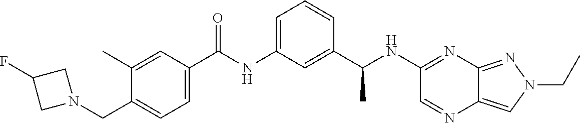 [SMILES: CCn1cc2ncc(N[C@@H](C)c3cccc(NC(=O)c4ccc(CN5CC(F)C5)c(C)c4)c3)nc2n1] | 487.57 | 488.2 |
| 498 | 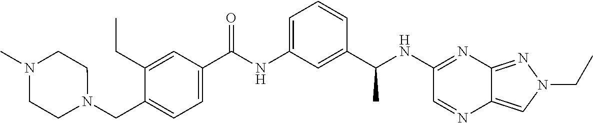 [SMILES: CCc1cc(C(=O)Nc2cccc([C@H](C)Nc3cnc4cn(CC)nc4n3)c2)ccc1CN1CCN(C)CC1] | 526.67 | 527.3 |
| 499 | 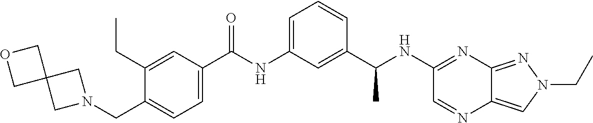 [SMILES: CCc1cc(C(=O)Nc2cccc([C@H](C)Nc3cnc4cn(CC)nc4n3)c2)ccc1CN1CC2(COC2)C1] | 525.64 | 526.2 |
| 500 | 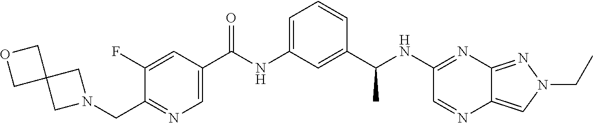 [SMILES: CCn1cc2ncc(N[C@@H](C)c3cccc(NC(=O)c4cnc(CN5CC6(COC6)C5)c(F)c4)c3)nc2n1] | 516.57 | 517.2 |
| 501 | 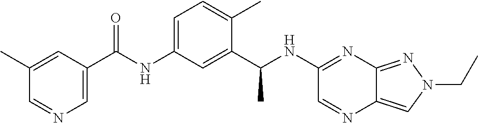 [SMILES: CCn1cc2ncc(N[C@@H](C)c3cc(NC(=O)c4cncc(C)c4)ccc3C)nc2n1] | 414.5 | 415.2 |
| 502 | 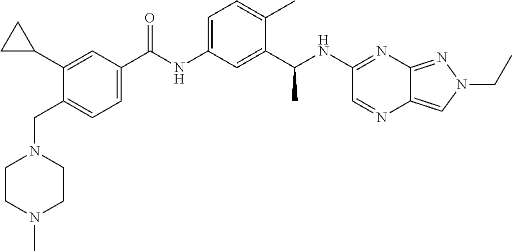 [SMILES: CCn1cc2ncc(N[C@@H](C)c3cc(NC(=O)c4ccc(CN5CCN(C)CC5)c(C5CC5)c4)ccc3C)nc2n1] | 538.68 | 539.5 |
| 503 | 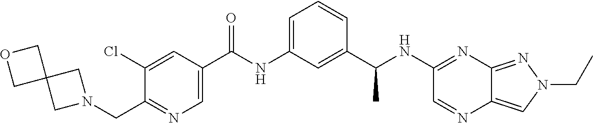 [SMILES: CCn1cc2ncc(N[C@@H](C)c3cccc(NC(=O)c4cnc(CN5CC6(COC6)C5)c(Cl)c4)c3)nc2n1] | 533.02 | 533.1 |
| 504 | 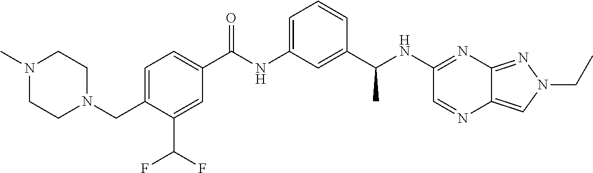 [SMILES: CCn1cc2ncc(N[C@@H](C)c3cccc(NC(=O)c4ccc(CN5CCN(C)CC5)c(C(F)F)c4)c3)nc2n1] | 548.63 | 549.3 |
| 505 | 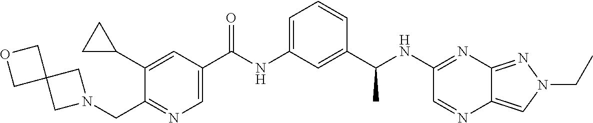 [SMILES: CCn1cc2ncc(N[C@@H](C)c3cccc(NC(=O)c4cnc(CN5CC6(COC6)C5)c(C5CC5)c4)c3)nc2n1] | 537.65 | 538.2 |
| 506 | 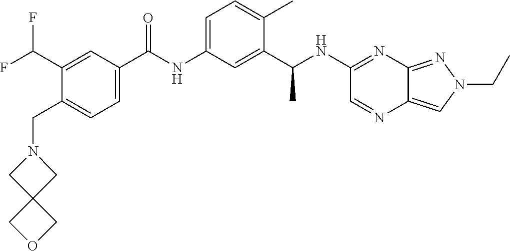 [SMILES: CCn1cc2ncc(N[C@@H](C)c3cc(NC(=O)c4ccc(CN5CC6(COC6)C5)c(C(F)F)c4)ccc3C)nc2n1] | 547.6 | 548.2 |
| 507 | 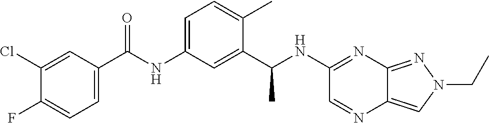 [SMILES: CCn1cc2ncc(N[C@@H](C)c3cc(NC(=O)c4ccc(F)c(Cl)c4)ccc3C)nc2n1] | 452.91 | 453.4 |
| 508 | 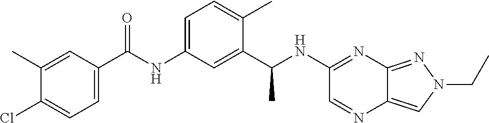 [SMILES: CCn1cc2ncc(N[C@@H](C)c3cc(NC(=O)c4ccc(Cl)c(C)c4)ccc3C)nc2n1] | 448.95 | 450.6 |
| 509 | 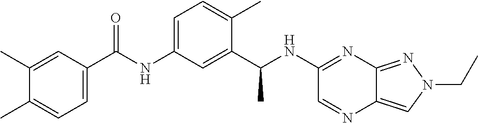 [SMILES: CCn1cc2ncc(N[C@@H](C)c3cc(NC(=O)c4ccc(C)c(C)c4)ccc3C)nc2n1] | 429.52 | 430.2 |
| 510 | 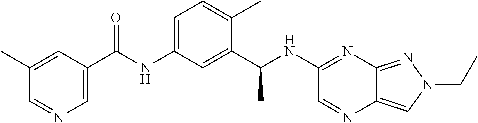 [SMILES: CCn1cc2ncc(N[C@@H](C)c3cc(NC(=O)c4cncc(C)c4)ccc3C)nc2n1] | 415.49 | 416.5 |
| 511 | 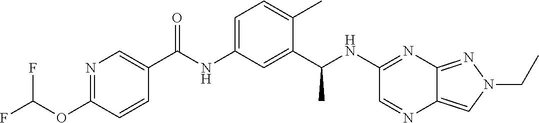 [SMILES: CCn1cc2ncc(N[C@@H](C)c3cc(NC(=O)c4ccc(OC(F)F)nc4)ccc3C)nc2n1] | 467.47 | 469.1 |
| 512 | 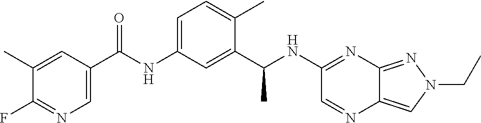 [SMILES: CCn1cc2ncc(N[C@@H](C)c3cc(NC(=O)c4cnc(F)c(C)c4)ccc3C)nc2n1] | 433.48 | 435.2 |
| 513 | 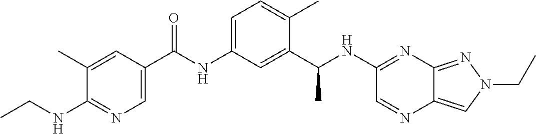 [SMILES: CCNc1ncc(C(=O)Nc2ccc(C)c([C@H](C)Nc3cnc4cn(CC)nc4n3)c2)cc1C] | 458.56 | 459.5 |
| 514 | 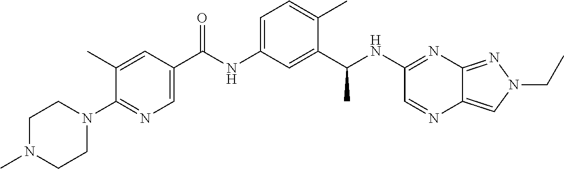 [SMILES: CCn1cc2ncc(N[C@@H](C)c3cc(NC(=O)c4cnc(N5CCN(C)CC5)c(C)c4)ccc3C)nc2n1] | 513.64 | 514.5 |
| 515 | 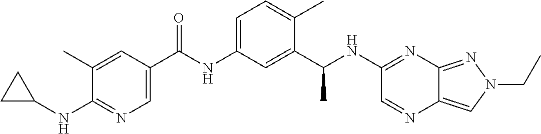 [SMILES: CCn1cc2ncc(N[C@@H](C)c3cc(NC(=O)c4cnc(NC5CC5)c(C)c4)ccc3C)nc2n1] | 470.57 | 471.4 |
| 516 | 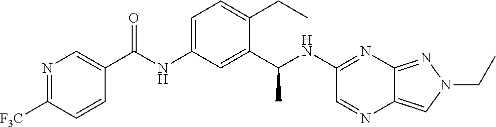 [SMILES: CCc1ccc(NC(=O)c2ccc(C(F)(F)F)nc2)cc1[C@H](C)Nc1cnc2cn(CC)nc2n1] | 483.49 | 484.1 |
| 517 | 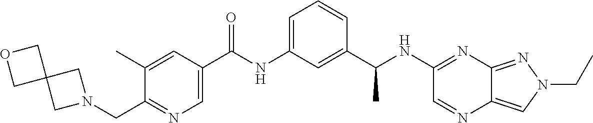 [SMILES: CCn1cc2ncc(N[C@@H](C)c3cccc(NC(=O)c4cnc(CN5CC6(COC6)C5)c(C)c4)c3)nc2n1] | 512.61 | 513.2 |
| 518 | 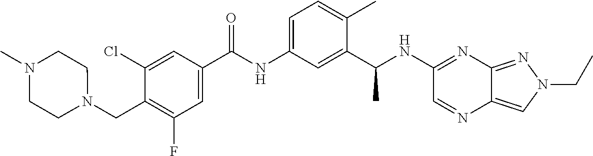 [SMILES: CCn1cc2ncc(N[C@@H](C)c3cc(NC(=O)c4cc(F)c(CN5CCN(C)CC5)c(Cl)c4)ccc3C)nc2n1] | 565.08 | 565.2 |
| 519 | 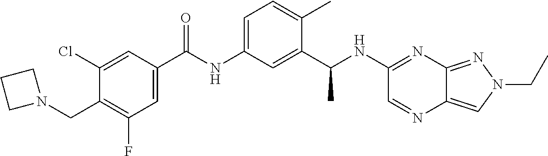 [SMILES: CCn1cc2ncc(N[C@@H](C)c3cc(NC(=O)c4cc(F)c(CN5CCC5)c(Cl)c4)ccc3C)nc2n1] | 522.02 | 522.2 |
| 520 | 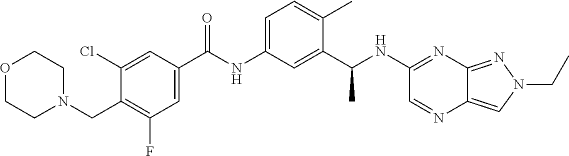 [SMILES: CCn1cc2ncc(N[C@@H](C)c3cc(NC(=O)c4cc(F)c(CN5CCOCC5)c(Cl)c4)ccc3C)nc2n1] | 552.04 | 552.2 |
| 521 | 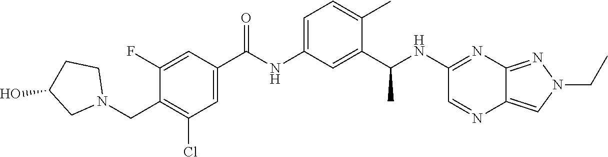 [SMILES: CCn1cc2ncc(N[C@@H](C)c3cc(NC(=O)c4cc(F)c(CN5CC[C@@H](O)C5)c(Cl)c4)ccc3C)nc2n1] | 552.04 | 552.2 |
| 522 | 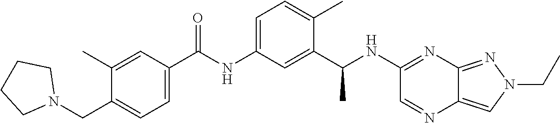 [SMILES: CCn1cc2ncc(N[C@@H](C)c3cc(NC(=O)c4ccc(CN5CCCC5)c(C)c4)ccc3C)nc2n1] | 497.63 | 498.5 |
| 523 | 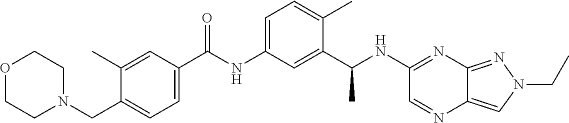 [SMILES: CCn1cc2ncc(N[C@@H](C)c3cc(NC(=O)c4ccc(CN5CCOCC5)c(C)c4)ccc3C)nc2n1] | 513.63 | 514.3 |
| 524 | 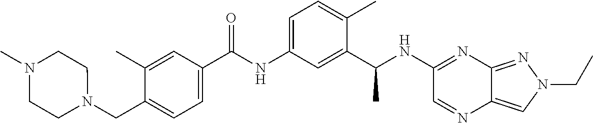 [SMILES: CCn1cc2ncc(N[C@@H](C)c3cc(NC(=O)c4ccc(CN5CCN(C)CC5)c(C)c4)ccc3C)nc2n1] | 526.67 | 527.7 |
| 525 | 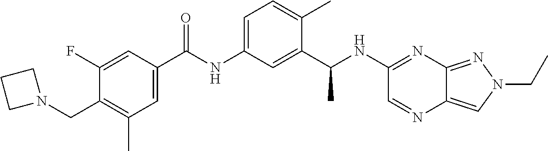 [SMILES: CCn1cc2ncc(N[C@@H](C)c3cc(NC(=O)c4cc(C)c(CN5CCC5)c(F)c4)ccc3C)nc2n1] | 501.6 | 502.1 |
| 526 | 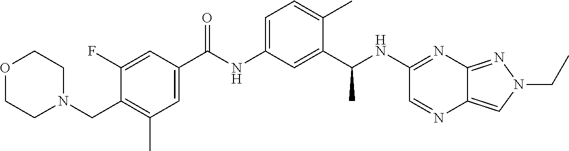 [SMILES: CCn1cc2ncc(N[C@@H](C)c3cc(NC(=O)c4cc(C)c(CN5CCOCC5)c(F)c4)ccc3C)nc2n1] | 531.62 | 532.1 |
| 527 | 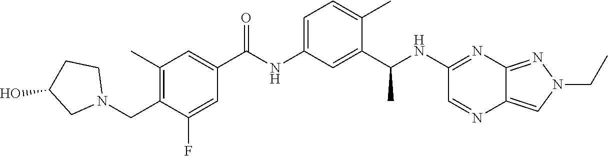 [SMILES: CCn1cc2ncc(N[C@@H](C)c3cc(NC(=O)c4cc(C)c(CN5CC[C@@H](O)C5)c(F)c4)ccc3C)nc2n1] | 531.62 | 532.2 |
| 528 | 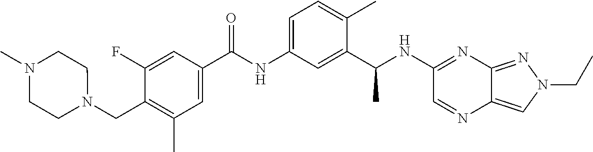 [SMILES: CCn1cc2ncc(N[C@@H](C)c3cc(NC(=O)c4cc(C)c(CN5CCN(C)CC5)c(F)c4)ccc3C)nc2n1] | 544.66 | 545.2 |
| 529 | 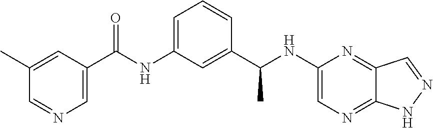 [SMILES: Cc1cncc(C(=O)Nc2cccc([C@H](C)Nc3cnc4[nH]ncc4n3)c2)c1] | 373.42 | 374.20 |
| 530 | 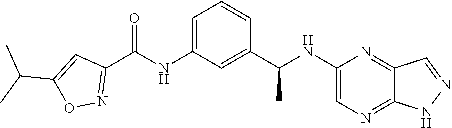 [SMILES: CC(C)c1cc(C(=O)Nc2cccc([C@H](C)Nc3cnc4[nH]ncc4n3)c2)no1] | 391.44 | 392.20 |
| 531 | 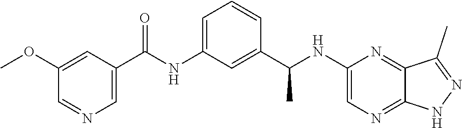 [SMILES: COc1cncc(C(=O)Nc2cccc([C@H](C)Nc3cnc4[nH]nc(C)c4n3)c2)c1] | 403.45 | 404.20 |
| 532 | 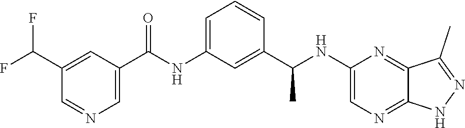 [SMILES: Cc1n[nH]c2ncc(N[C@@H](C)c3cccc(NC(=O)c4cncc(C(F)F)c4)c3)nc12] | 423.43 | 424.20 |
| 533 | 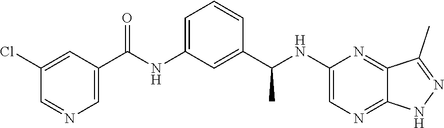 [SMILES: Cc1n[nH]c2ncc(N[C@@H](C)c3cccc(NC(=O)c4cncc(Cl)c4)c3)nc12] | 407.86 | 408.15 |
| 534 | 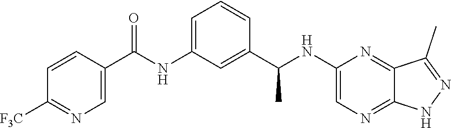 [SMILES: Cc1n[nH]c2ncc(N[C@@H](C)c3cccc(NC(=O)c4ccc(C(F)(F)F)nc4)c3)nc12] | 441.42 | 442.20 |
| 535 | 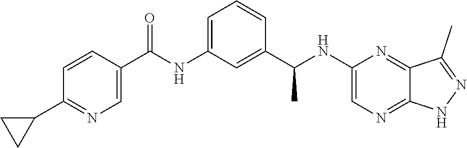 [SMILES: Cc1n[nH]c2ncc(N[C@@H](C)c3cccc(NC(=O)c4ccc(C5CC5)nc4)c3)nc12] | 413.49 | 414.20 |
| 536 | 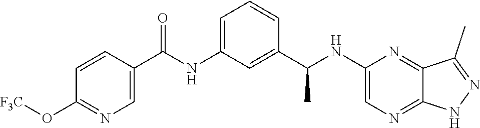 [SMILES: Cc1n[nH]c2ncc(N[C@@H](C)c3cccc(NC(=O)c4ccc(OC(F)(F)F)nc4)c3)nc12] | 457.42 | 458.20 |
| 537 | 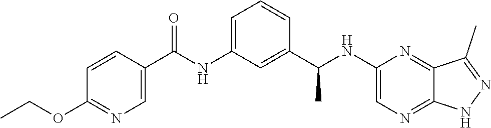 [SMILES: CCOc1ccc(C(=O)Nc2cccc([C@H](C)Nc3cnc4[nH]nc(C)c4n3)c2)cn1] | 417.47 | 418.30 |
| 538 | 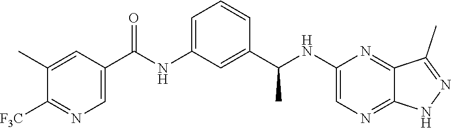 [SMILES: Cc1cc(C(=O)Nc2cccc([C@H](C)Nc3cnc4[nH]nc(C)c4n3)c2)cnc1C(F)(F)F] | 455.45 | 456.20 |
| 539 | 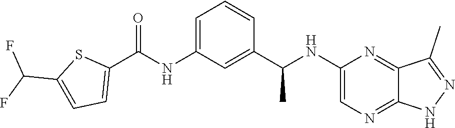 [SMILES: Cc1n[nH]c2ncc(N[C@@H](C)c3cccc(NC(=O)c4ccc(C(F)F)s4)c3)nc12] | 428.46 | 429.15 |
| 540 | 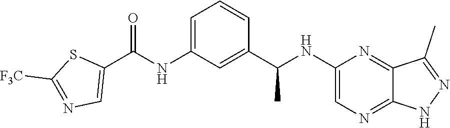 [SMILES: Cc1n[nH]c2ncc(N[C@@H](C)c3cccc(NC(=O)c4cnc(C(F)(F)F)s4)c3)nc12] | 447.44 | 448.20 |
| 541 | 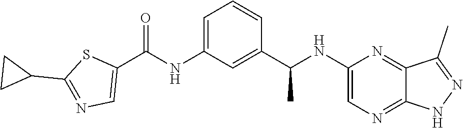 [SMILES: Cc1n[nH]c2ncc(N[C@@H](C)c3cccc(NC(=O)c4cnc(C5CC5)s4)c3)nc12] | 419.51 | 420.25 |
| 542 | 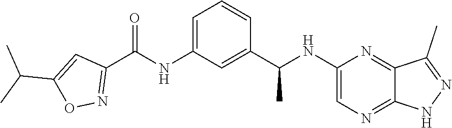 [SMILES: Cc1n[nH]c2ncc(N[C@@H](C)c3cccc(NC(=O)c4cc(C(C)C)on4)c3)nc12] | 405.46 | 406.25 |
| 543 | 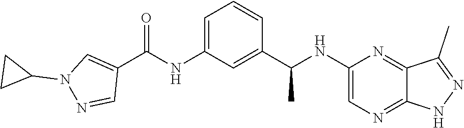 [SMILES: Cc1n[nH]c2ncc(N[C@@H](C)c3cccc(NC(=O)c4cnn(C5CC5)c4)c3)nc12] | 402.46 | 403.20 |
| 544 | 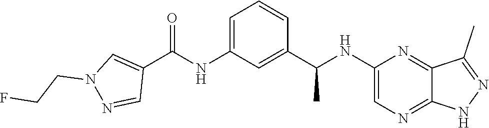 [SMILES: Cc1n[nH]c2ncc(N[C@@H](C)c3cccc(NC(=O)c4cnn(CCF)c4)c3)nc12] | 408.44 | 409.25 |
| 545 | 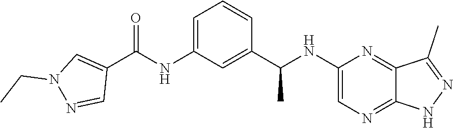 [SMILES: CCn1cc(C(=O)Nc2cccc([C@H](C)Nc3cnc4[nH]nc(C)c4n3)c2)cn1] | 390.45 | 391.20 |
| 546 | 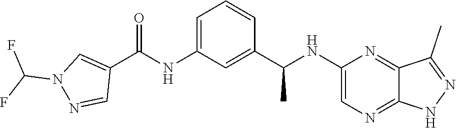 [SMILES: Cc1n[nH]c2ncc(N[C@@H](C)c3cccc(NC(=O)c4cnn(C(F)F)c4)c3)nc12] | 412.4 | 413.20 |
| 547 | 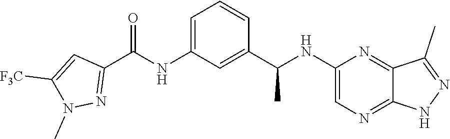 [SMILES: Cc1n[nH]c2ncc(N[C@@H](C)c3cccc(NC(=O)c4cc(C(F)(F)F)n(C)n4)c3)nc12] | 444.42 | 445.25 |
| 548 | 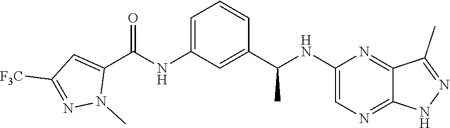 [SMILES: Cc1n[nH]c2ncc(N[C@@H](C)c3cccc(NC(=O)c4cc(C(F)(F)F)nn4C)c3)nc12] | 444.42 | 445.25 |
| 549 | 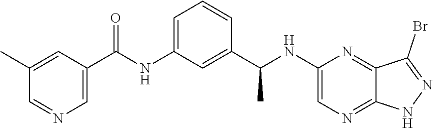 [SMILES: Cc1cncc(C(=O)Nc2cccc([C@H](C)Nc3cnc4[nH]nc(Br)c4n3)c2)c1] | 452.32 | 454.20 |
| 550 | 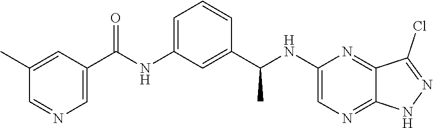 [SMILES: Cc1cncc(C(=O)Nc2cccc([C@H](C)Nc3cnc4[nH]nc(Cl)c4n3)c2)c1] | 407.86 | 408.1 |
| 551 | 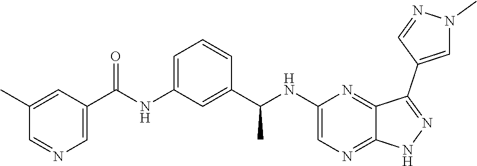 | 453.5 | 454.2 |
| 552 | 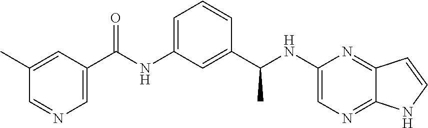 [SMILES: Cc1cncc(C(=O)Nc2cccc([C@H](C)Nc3cnc4[nH]ccc4n3)c2)c1] | 372.42 | 373.15 |
| 553 | 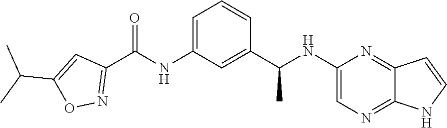 [SMILES: CC(C)c1cc(C(=O)Nc2cccc([C@H](C)Nc3cnc4[nH]ccc4n3)c2)no1] | 390.44 | 391.20 |
| 554 | 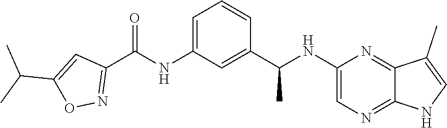 [SMILES: Cc1c[nH]c2ncc(N[C@@H](C)c3cccc(NC(=O)c4cc(C(C)C)on4)c3)nc12] | 404.46 | 405.20 |
| 555 | 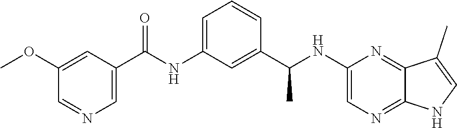 [SMILES: COc1cncc(C(=O)Nc2cccc([C@H](C)Nc3cnc4[nH]cc(C)c4n3)c2)c1] | 402.46 | 403.00 |
| 556 | 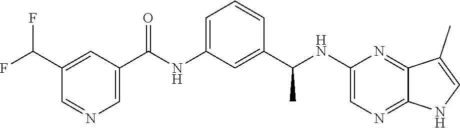 [SMILES: Cc1c[nH]c2ncc(N[C@@H](C)c3cccc(NC(=O)c4cncc(C(F)F)c4)c3)nc12] | 422.44 | 423.20 |
| 557 | 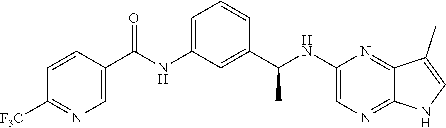 [SMILES: Cc1c[nH]c2ncc(N[C@@H](C)c3cccc(NC(=O)c4ccc(C(F)(F)F)nc4)c3)nc12] | 440.43 | 441.00 |
| 558 | 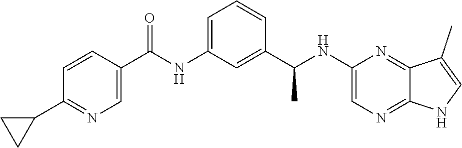 [SMILES: Cc1c[nH]c2ncc(N[C@@H](C)c3cccc(NC(=O)c4ccc(C5CC5)nc4)c3)nc12] | 412.5 | 413.10 |
| 559 | 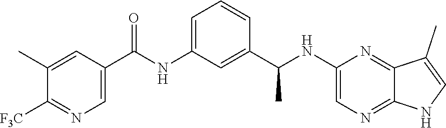 [SMILES: Cc1cc(C(=O)Nc2cccc([C@H](C)Nc3cnc4[nH]cc(C)c4n3)c2)cnc1C(F)(F)F] | 454.46 | 455.00 |
| 560 | 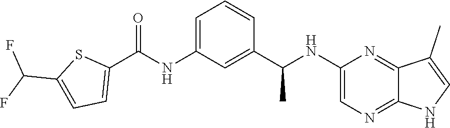 [SMILES: Cc1c[nH]c2ncc(N[C@@H](C)c3cccc(NC(=O)c4ccc(C(F)F)s4)c3)nc12] | 427.47 | 427.90 |
| 561 | 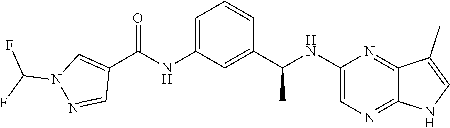 [SMILES: Cc1c[nH]c2ncc(N[C@@H](C)c3cccc(NC(=O)c4cnn(C(F)F)c4)c3)nc12] | 411.42 | 412.20 |
| 562 | 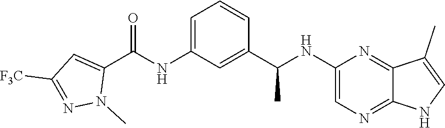 [SMILES: Cc1c[nH]c2ncc(N[C@@H](C)c3cccc(NC(=O)c4cc(C(F)(F)F)nn4C)c3)nc12] | 443.43 | 444.20 |
| 563 | 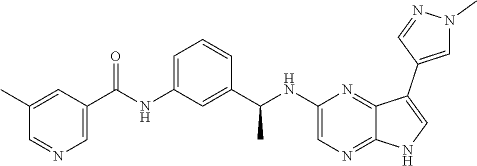 | 452.52 | 453.30 |
| 564 | 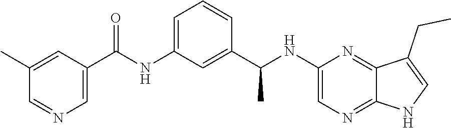 [SMILES: CCc1c[nH]c2ncc(N[C@@H](C)c3cccc(NC(=O)c4cncc(C)c4)c3)nc12] | 400.48 | 401.3 |
| 565 | 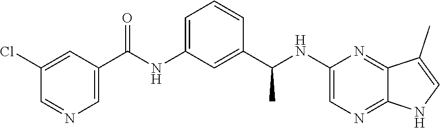 [SMILES: Cc1c[nH]c2ncc(N[C@@H](C)c3cccc(NC(=O)c4cncc(Cl)c4)c3)nc12] | 406.87 | 407.2 |
| 566 | 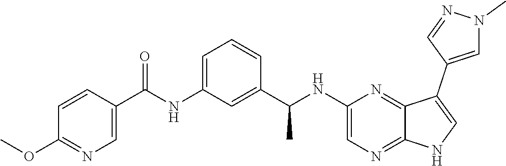 | 468.51 | 469.3 |
| 567 | 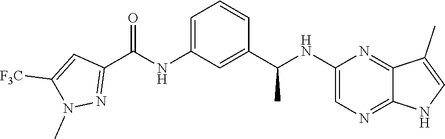 [SMILES: Cc1c[nH]c2ncc(N[C@@H](C)c3cccc(NC(=O)c4cc(C(F)(F)F)n(C)n4)c3)nc12] | 443.42 | 444.2 |
| 568 | 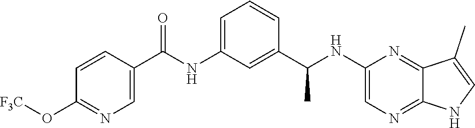 [SMILES: Cc1c[nH]c2ncc(N[C@@H](C)c3cccc(NC(=O)c4ccc(OC(F)(F)F)nc4)c3)nc12] | 456.42 | 457.1 |
| 569 | 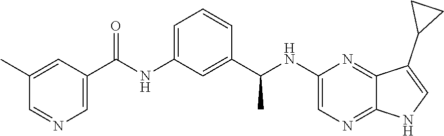 [SMILES: Cc1cncc(C(=O)Nc2cccc([C@H](C)Nc3cnc4[nH]cc(C5CC5)c4n3)c2)c1] | 412.49 | 413.2 |
| 570 | 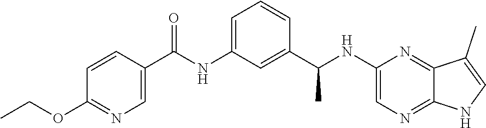 [SMILES: CCOc1ccc(C(=O)Nc2cccc([C@H](C)Nc3cnc4[nH]cc(C)c4n3)c2)cn1] | 416.47 | 417.2 |
| 571 | 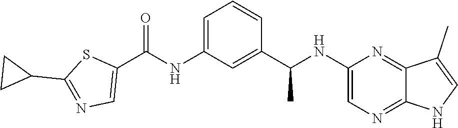 [SMILES: Cc1c[nH]c2ncc(N[C@@H](C)c3cccc(NC(=O)c4cnc(C5CC5)s4)c3)nc12] | 418.51 | 419.1 |
| 572 | 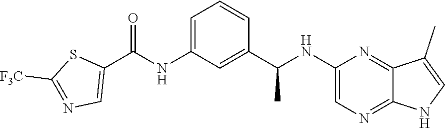 [SMILES: Cc1c[nH]c2ncc(N[C@@H](C)c3cccc(NC(=O)c4cnc(C(F)(F)F)s4)c3)nc12] | 446.45 | 447.1 |
| 573 | 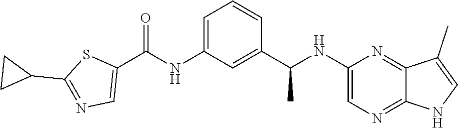 [SMILES: Cc1c[nH]c2ncc(N[C@@H](C)c3cccc(NC(=O)c4cnc(C5CC5)s4)c3)nc12] | 401.46 | 402.2 |
| 574 | 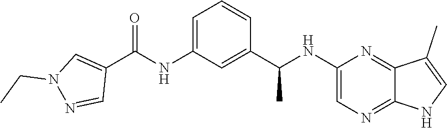 [SMILES: CCn1cc(C(=O)Nc2cccc([C@H](C)Nc3cnc4[nH]cc(C)c4n3)c2)cn1] | 389.45 | 390.2 |
| 575 | 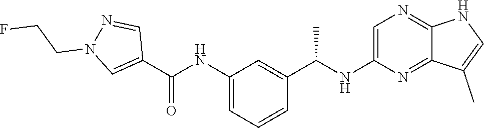 [SMILES: Cc1c[nH]c2ncc(N[C@@H](C)c3cccc(NC(=O)c4cnn(CCF)c4)c3)nc12] | 407.44 | 408.2 |
| 576 | 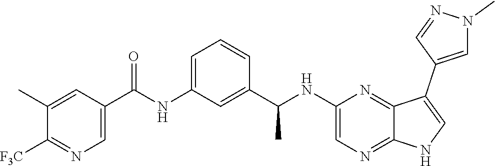 [SMILES: Cc1cc(C(=O)Nc2cccc([C@H](C)Nc3cnc4[nH]cc(-c5cnn(C)c5)c4n3)c2)cnc1C(F)(F)F] | 520.51 | 521.3 |
| 577 | 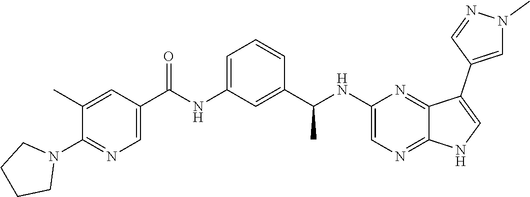 [SMILES: Cc1cc(C(=O)Nc2cccc([C@H](C)Nc3cnc4[nH]cc(-c5cnn(C)c5)c4n3)c2)cnc1N1CCCC1] | 521.62 | 522.3 |
| 578 | 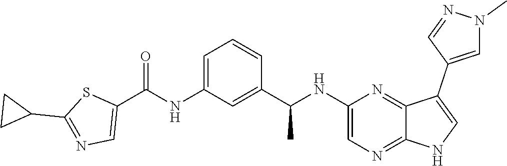 [SMILES: C[C@H](Nc1cnc2[nH]cc(-c3cnn(C)c3)c2n1)c1cccc(NC(=O)c2cnc(C3CC3)s2)c1] | 484.58 | 485.2 |
| 579 | 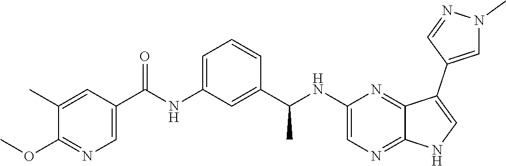 | 482.54 | 483.3 |
| 580 | 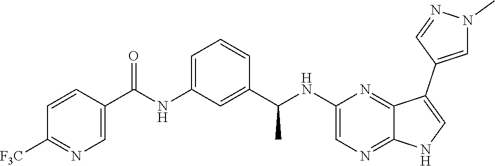 [SMILES: C[C@H](Nc1cnc2[nH]cc(-c3cnn(C)c3)c2n1)c1cccc(NC(=O)c2ccc(C(F)(F)F)nc2)c1] | 506.48 | 507.2 |
| 581 | 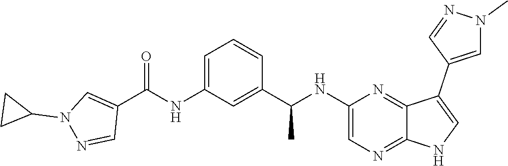 [SMILES: C[C@H](Nc1cnc2[nH]cc(-c3cnn(C)c3)c2n1)c1cccc(NC(=O)c2cnn(C3CC3)c2)c1] | 467.53 | 468.3 |
| 582 | 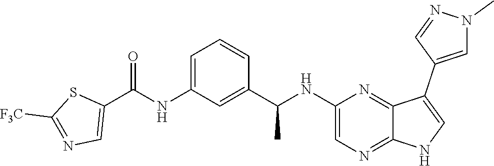 [SMILES: C[C@H](Nc1cnc2[nH]cc(-c3cnn(C)c3)c2n1)c1cccc(NC(=O)c2cnc(C(F)(F)F)s2)c1] | 512.51 | 513.2 |
| 583 | 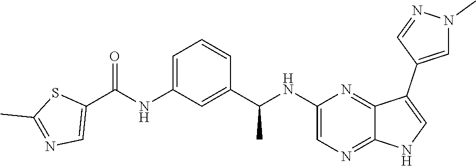 | 458.54 | 459.3 |
| 584 | 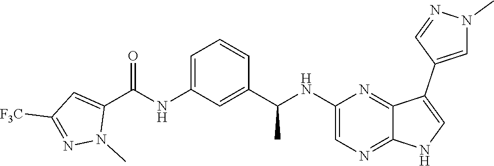 [SMILES: C[C@H](Nc1cnc2[nH]cc(-c3cnn(C)c3)c2n1)c1cccc(NC(=O)c2cc(C(F)(F)F)nn2C)c1] | 509.49 | 510.3 |
| 585 | 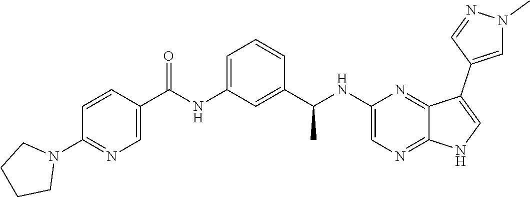 [SMILES: C[C@H](Nc1cnc2[nH]cc(-c3cnn(C)c3)c2n1)c1cccc(NC(=O)c2ccc(N3CCCC3)nc2)c1] | 507.59 | 508.3 |
| 586 | 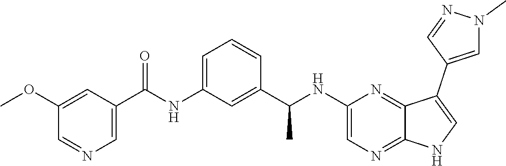 | 468.51 | 469.3 |
| 587 | 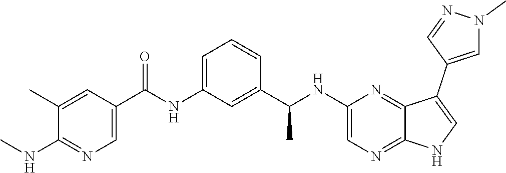 | 481.55 | 482.3 |
| 588 | 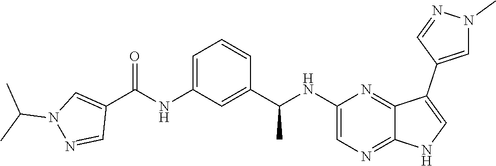 [SMILES: CC(C)n1cc(C(=O)Nc2cccc([C@H](C)Nc3cnc4[nH]cc(-c5cnn(C)c5)c4n3)c2)cn1] | 469.54 | 470.3 |
| 589 | 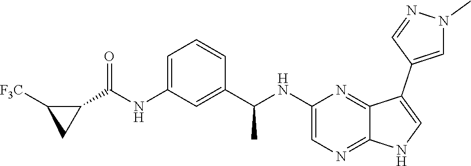 [SMILES: C[C@H](Nc1cnc2[nH]cc(-c3cnn(C)c3)c2n1)c1cccc(NC(=O)[C@@H]2C[C@H]2C(F)(F)F)c1] | 469.46 | 470.3 |
| 590 | 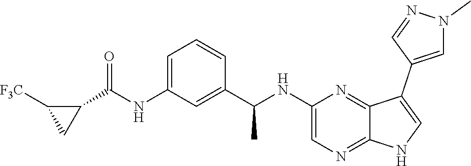 [SMILES: C[C@H](Nc1cnc2[nH]cc(-c3cnn(C)c3)c2n1)c1cccc(NC(=O)[C@@H]2C[C@@H]2C(F)(F)F)c1] | 469.46 | 470.3 |
| 591 | 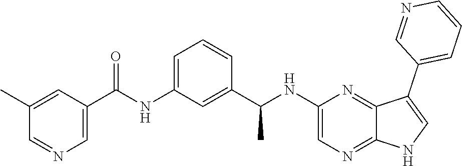 [SMILES: Cc1cncc(C(=O)Nc2cccc([C@H](C)Nc3cnc4[nH]cc(-c5cccnc5)c4n3)c2)c1] | 449.51 | 450.3 |
| 592 | 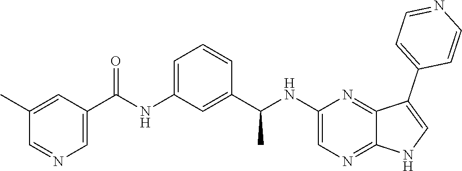 [SMILES: Cc1cncc(C(=O)Nc2cccc([C@H](C)Nc3cnc4[nH]cc(-c5ccncc5)c4n3)c2)c1] | 449.51 | 450.2 |
| 593 | 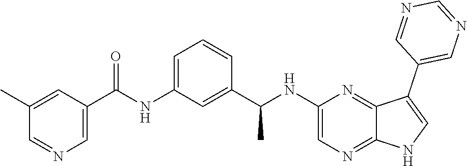 [SMILES: Cc1cncc(C(=O)Nc2cccc([C@H](C)Nc3cnc4[nH]cc(-c5cncnc5)c4n3)c2)c1] | 450.49 | 451.3 |
| 594 | 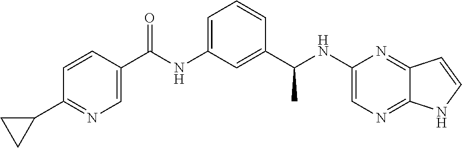 [SMILES: C[C@H](Nc1cnc2[nH]ccc2n1)c1cccc(NC(=O)c2ccc(C3CC3)nc2)c1] | 398.46 | 399.2 |
| 595 | 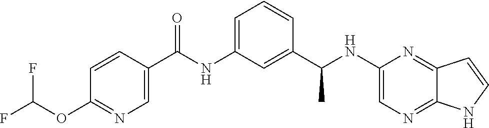 [SMILES: C[C@H](Nc1cnc2[nH]ccc2n1)c1cccc(NC(=O)c2ccc(OC(F)F)nc2)c1] | 424.4 | 425.2 |
| 596 | 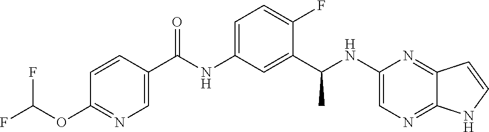 [SMILES: C[C@H](Nc1cnc2[nH]ccc2n1)c1cc(NC(=O)c2ccc(OC(F)F)nc2)ccc1F] | 442.39 | 443.2 |
| 597 | 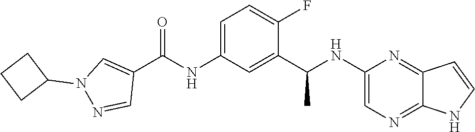 [SMILES: C[C@H](Nc1cnc2[nH]ccc2n1)c1cc(NC(=O)c2cnn(C3CCC3)c2)ccc1F] | 419.45 | 420.3 |
| 598 | 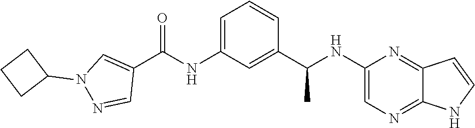 [SMILES: C[C@H](Nc1cnc2[nH]ccc2n1)c1cccc(NC(=O)c2cnn(C3CCC3)c2)c1] | 401.46 | 402.2 |
| 599 | 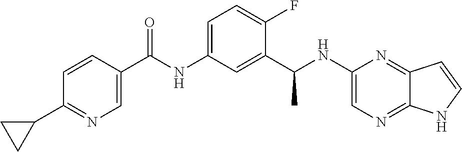 [SMILES: C[C@H](Nc1cnc2[nH]ccc2n1)c1cc(NC(=O)c2ccc(C3CC3)nc2)ccc1F] | 416.45 | 417.1 |
| 600 | 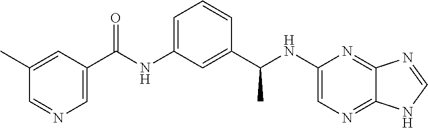 [SMILES: Cc1cncc(C(=O)Nc2cccc([C@H](C)Nc3cnc4[nH]cnc4n3)c2)c1] | 372.42 | 373.1 |
| 601 | 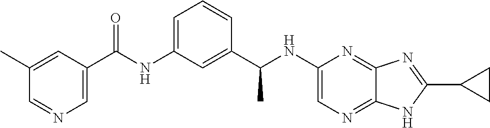 [SMILES: Cc1cncc(C(=O)Nc2cccc([C@H](C)Nc3cnc4[nH]c(C5CC5)nc4n3)c2)c1] | 412.49 | 413.2 |
| 602 | 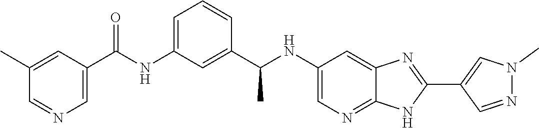 | 452.51 | 453.3 |
| 603 | 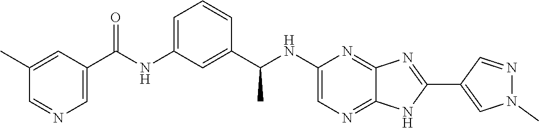 | 453.5 | 454.1 |
| 604 | 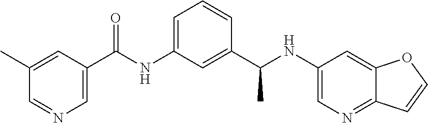 [SMILES: Cc1cncc(C(=O)Nc2cccc([C@H](C)Nc3cnc4ccoc4c3)c2)c1] | 372.43 | 373.10 |
| 605 | 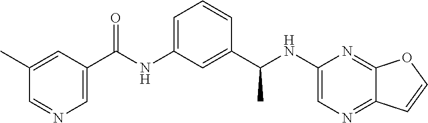 [SMILES: Cc1cncc(C(=O)Nc2cccc([C@H](C)Nc3cnc4ccoc4n3)c2)c1] | 373.42 | 374.20 |
| 606 | 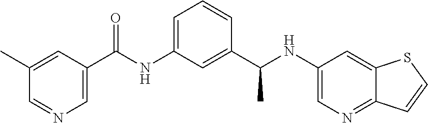 [SMILES: Cc1cncc(C(=O)Nc2cccc([C@H](C)Nc3cnc4ccsc4c3)c2)c1] | 388.49 | 389.10 |
| 607 | 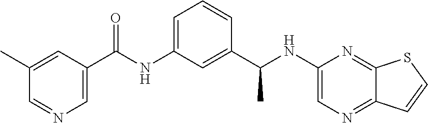 [SMILES: Cc1cncc(C(=O)Nc2cccc([C@H](C)Nc3cnc4ccsc4n3)c2)c1] | 389.48 | 390.15 |
| 608 | 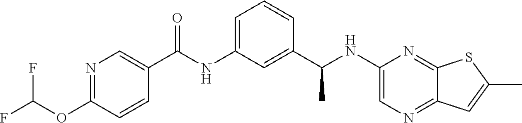 [SMILES: Cc1cc2ncc(N[C@@H](C)c3cccc(NC(=O)c4ccc(OC(F)F)nc4)c3)nc2s1] | 455.48 | 456.2 |
| 609 | 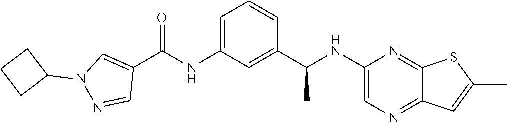 [SMILES: Cc1cc2ncc(N[C@@H](C)c3cccc(NC(=O)c4cnn(C5CCC5)c4)c3)nc2s1] | 432.54 | 433.2 |
| 610 | 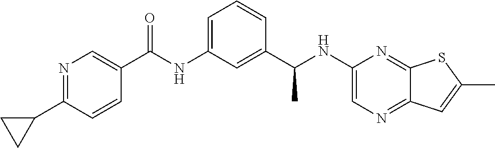 [SMILES: Cc1cc2ncc(N[C@@H](C)c3cccc(NC(=O)c4ccc(C5CC5)nc4)c3)nc2s1] | 429.54 | 430.1 |
| 611 | 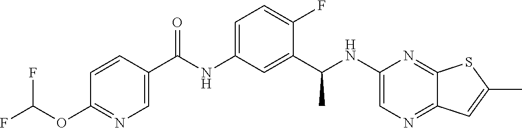 [SMILES: Cc1cc2ncc(N[C@@H](C)c3cc(NC(=O)c4ccc(OC(F)F)nc4)ccc3F)nc2s1] | 473.47 | 474.1 |
| 612 | 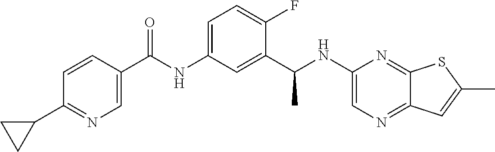 [SMILES: Cc1cc2ncc(N[C@@H](C)c3cc(NC(=O)c4ccc(C5CC5)nc4)ccc3F)nc2s1] | 447.53 | 448.2 |
| 613 | 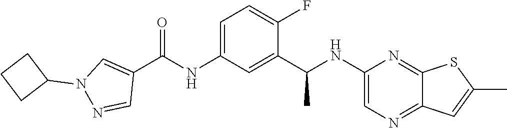 [SMILES: Cc1cc2ncc(N[C@@H](C)c3cc(NC(=O)c4cnn(C5CCC5)c4)ccc3F)nc2s1] | 450.53 | 451.1 |
| 614 | 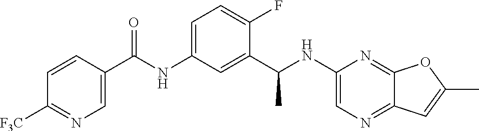 [SMILES: Cc1cc2ncc(N[C@@H](C)c3cc(NC(=O)c4ccc(C(F)(F)F)nc4)ccc3F)nc2o1] | 459.4 | 460 |
| 615 | 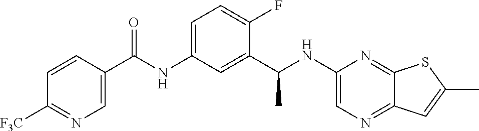 [SMILES: Cc1cc2ncc(N[C@@H](C)c3cc(NC(=O)c4ccc(C(F)(F)F)nc4)ccc3F)nc2s1] | 475.46 | 476 |
| 616 | 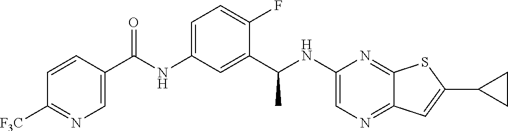 [SMILES: C[C@H](Nc1cnc2cc(C3CC3)sc2n1)c1cc(NC(=O)c2ccc(C(F)(F)F)nc2)ccc1F] | 501.5 | 502 |
| 617 | 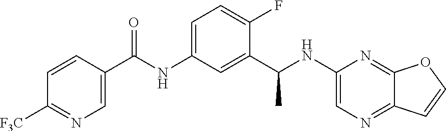 [SMILES: C[C@H](Nc1cnc2ccoc2n1)c1cc(NC(=O)c2ccc(C(F)(F)F)nc2)ccc1F] | 445.37 | 446.1 |
| 618 | 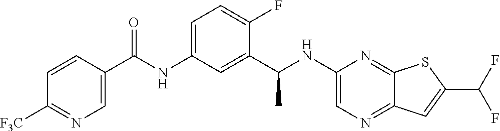 [SMILES: C[C@H](Nc1cnc2cc(C(F)F)sc2n1)c1cc(NC(=O)c2ccc(C(F)(F)F)nc2)ccc1F] | 511.44 | 512 |
| 619 | 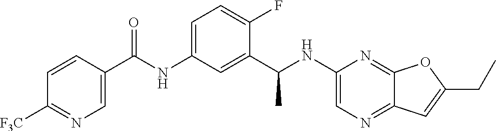 [SMILES: CCc1cc2ncc(N[C@@H](C)c3cc(NC(=O)c4ccc(C(F)(F)F)nc4)ccc3F)nc2o1] | 473.42 | 474.4 |
| 620 | 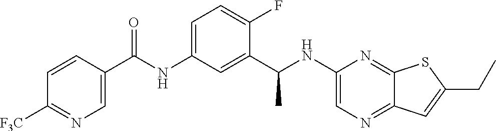 [SMILES: CCc1cc2ncc(N[C@@H](C)c3cc(NC(=O)c4ccc(C(F)(F)F)nc4)ccc3F)nc2s1] | 489.49 | 490.1 |
| 621 | 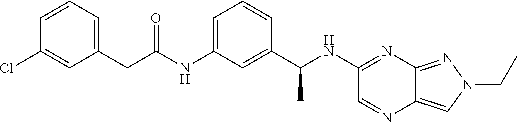 [SMILES: CCn1cc2ncc(N[C@@H](C)c3cccc(NC(=O)Cc4cccc(Cl)c4)c3)nc2n1] | 434.92 | 435.9 |
| 622 | 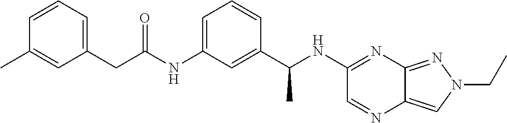 [SMILES: CCn1cc2ncc(N[C@@H](C)c3cccc(NC(=O)Cc4cccc(C)c4)c3)nc2n1] | 414.5 | 416.08 |
| 623 | 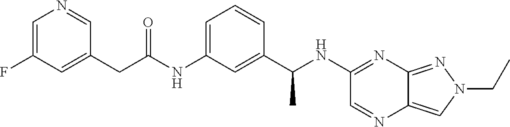 [SMILES: CCn1cc2ncc(N[C@@H](C)c3cccc(NC(=O)Cc4cncc(F)c4)c3)nc2n1] | 419.45 | 420.2 |
| 624 | 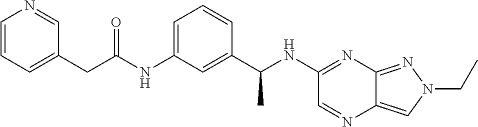 [SMILES: CCn1cc2ncc(N[C@@H](C)c3cccc(NC(=O)Cc4cccnc4)c3)nc2n1] | 401.46 | 402.2 |
| 625 | 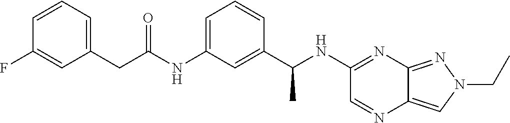 [SMILES: CCn1cc2ncc(N[C@@H](C)c3cccc(NC(=O)Cc4cccc(F)c4)c3)nc2n1] | 418.47 | 420.2 |
| 626 | 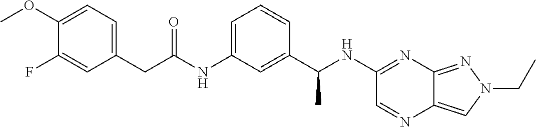 [SMILES: CCn1cc2ncc(N[C@@H](C)c3cccc(NC(=O)Cc4ccc(OC)c(F)c4)c3)nc2n1] | 448.49 | 450.6 |
| 627 | 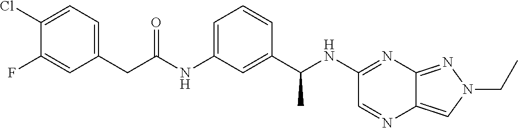 [SMILES: CCn1cc2ncc(N[C@@H](C)c3cccc(NC(=O)Cc4ccc(Cl)c(F)c4)c3)nc2n1] | 452.91 | 455.4 |
| 628 | 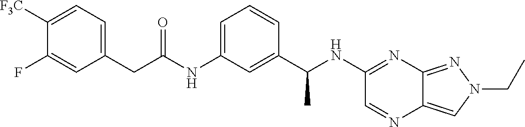 [SMILES: CCn1cc2ncc(N[C@@H](C)c3cccc(NC(=O)Cc4ccc(C(F)(F)F)c(F)c4)c3)nc2n1] | 486.46 | 488.6 |
| 629 | 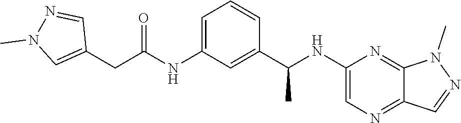 | 390.44 | 391.1 |
| 630 | 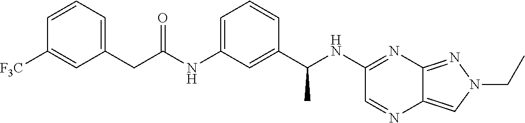 [SMILES: CCn1cc2ncc(N[C@@H](C)c3cccc(NC(=O)Cc4cccc(C(F)(F)F)c4)c3)nc2n1] | 468.47 | 470.26 |
| 631 | 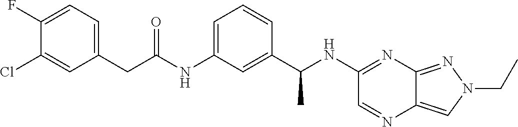 [SMILES: CCn1cc2ncc(N[C@@H](C)c3cccc(NC(=O)Cc4ccc(F)c(Cl)c4)c3)nc2n1] | 452.91 | 454.1 |
| 632 | 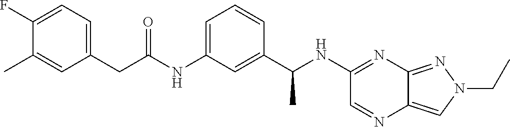 [SMILES: CCn1cc2ncc(N[C@@H](C)c3cccc(NC(=O)Cc4ccc(F)c(C)c4)c3)nc2n1] | 432.49 | 434.2 |
| 633 | 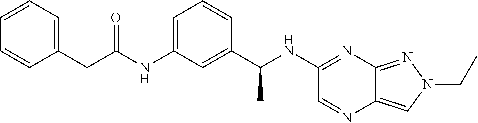 [SMILES: CCn1cc2ncc(N[C@@H](C)c3cccc(NC(=O)Cc4ccccc4)c3)nc2n1] | 400.48 | 401.8 |
| 634 | 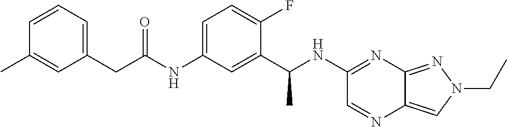 [SMILES: CCn1cc2ncc(N[C@@H](C)c3cc(NC(=O)Cc4cccc(C)c4)ccc3F)nc2n1] | 432.49 | 433.3 |
| 635 | 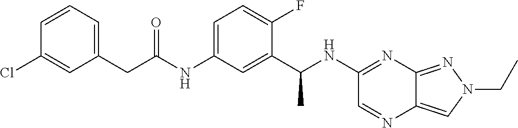 [SMILES: CCn1cc2ncc(N[C@@H](C)c3cc(NC(=O)Cc4cccc(Cl)c4)ccc3F)nc2n1] | 452.91 | 453.2 |
| 636 | 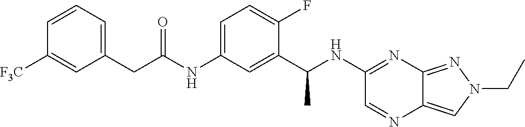 [SMILES: CCn1cc2ncc(N[C@@H](C)c3cc(NC(=O)Cc4cccc(C(F)(F)F)c4)ccc3F)nc2n1] | 486.46 | 487.2 |
| 637 | 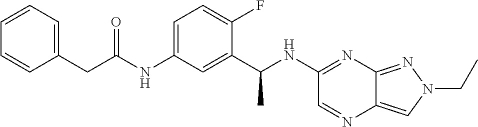 [SMILES: CCn1cc2ncc(N[C@@H](C)c3cc(NC(=O)Cc4ccccc4)ccc3F)nc2n1] | 418.47 | 419.3 |
| 638 | 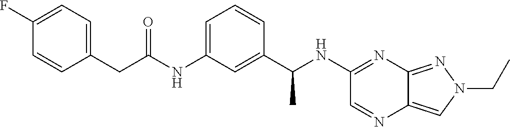 [SMILES: CCn1cc2ncc(N[C@@H](C)c3cccc(NC(=O)Cc4ccc(F)cc4)c3)nc2n1] | 418.47 | 419.1 |
| 639 | 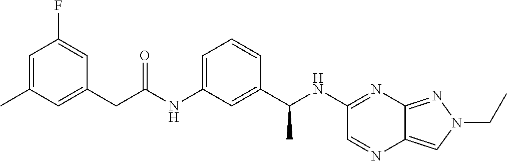 [SMILES: CCn1cc2ncc(N[C@@H](C)c3cccc(NC(=O)Cc4cc(C)cc(F)c4)c3)nc2n1] | 432.49 | 433.5 |
| 640 | 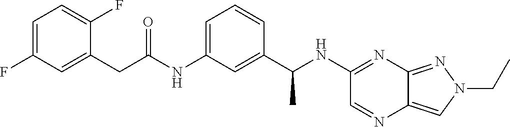 [SMILES: CCn1cc2ncc(N[C@@H](C)c3cccc(NC(=O)Cc4cc(F)ccc4F)c3)nc2n1] | 436.46 | 437.1 |
| 641 | 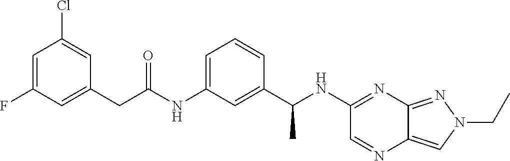 [SMILES: CCn1cc2ncc(N[C@@H](C)c3cccc(NC(=O)Cc4cc(F)cc(Cl)c4)c3)nc2n1] | 452.91 | 453.2 |
| 642 | 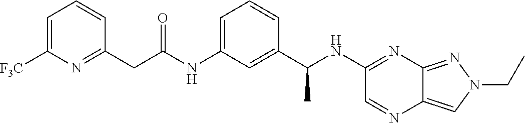 [SMILES: CCn1cc2ncc(N[C@@H](C)c3cccc(NC(=O)Cc4cccc(C(F)(F)F)n4)c3)nc2n1] | 469.46 | 470.5 |
| 643 | 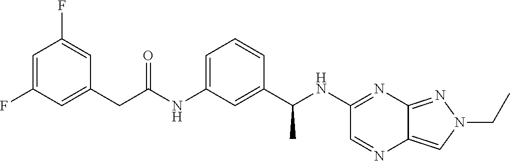 [SMILES: CCn1cc2ncc(N[C@@H](C)c3cccc(NC(=O)Cc4cc(F)cc(F)c4)c3)nc2n1] | 436.46 | 437.1 |
| 644 | 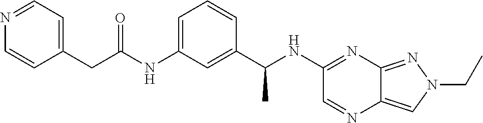 [SMILES: CCn1cc2ncc(N[C@@H](C)c3cccc(NC(=O)Cc4ccncc4)c3)nc2n1] | 401.46 | 402 |
| 645 | 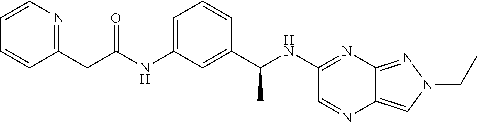 [SMILES: CCn1cc2ncc(N[C@@H](C)c3cccc(NC(=O)Cc4ccccn4)c3)nc2n1] | 401.46 | 402.1 |
| 646 | 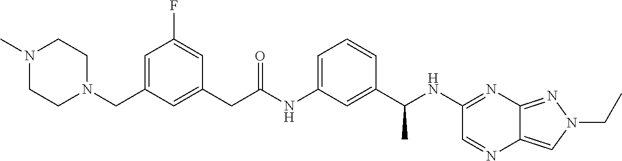 [SMILES: CCn1cc2ncc(N[C@@H](C)c3cccc(NC(=O)Cc4cc(F)cc(CN5CCN(C)CC5)c4)c3)nc2n1] | 530.64 | 531.2 |
| 647 | 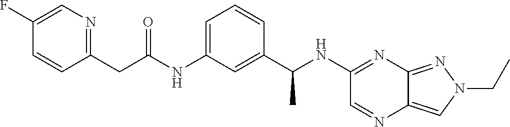 [SMILES: CCn1cc2ncc(N[C@@H](C)c3cccc(NC(=O)Cc4ccc(F)cn4)c3)nc2n1] | 419.45 | 420.4 |
| 648 | 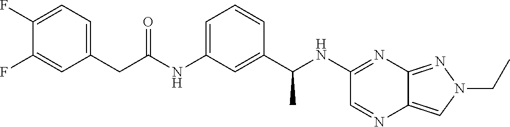 [SMILES: CCn1cc2ncc(N[C@@H](C)c3cccc(NC(=O)Cc4ccc(F)c(F)c4)c3)nc2n1] | 436.46 | 438.3 |
| 649 | 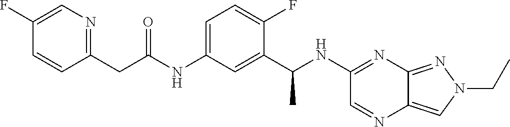 [SMILES: CCn1cc2ncc(N[C@@H](C)c3cc(NC(=O)Cc4ccc(F)cn4)ccc3F)nc2n1] | 437.44 | 438.1 |
| 650 | 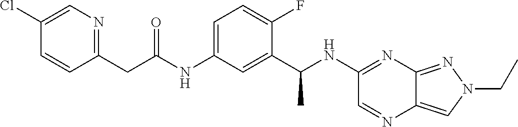 [SMILES: CCn1cc2ncc(N[C@@H](C)c3cc(NC(=O)Cc4ccc(Cl)cn4)ccc3F)nc2n1] | 453.9 | 454.1 |
| 651 | 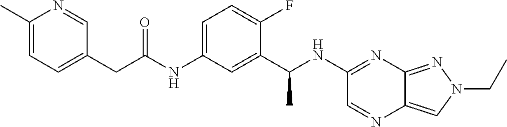 [SMILES: CCn1cc2ncc(N[C@@H](C)c3cc(NC(=O)Cc4ccc(C)nc4)ccc3F)nc2n1] | 433.48 | 434.1 |
| 652 | 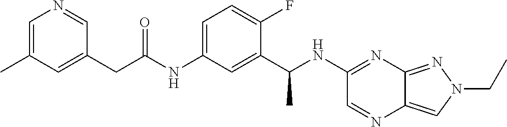 [SMILES: CCn1cc2ncc(N[C@@H](C)c3cc(NC(=O)Cc4cncc(C)c4)ccc3F)nc2n1] | 433.48 | 434.1 |
| 653 | 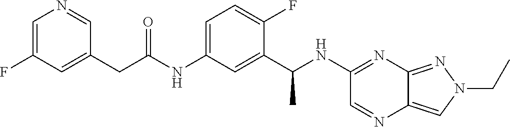 [SMILES: CCn1cc2ncc(N[C@@H](C)c3cc(NC(=O)Cc4cncc(F)c4)ccc3F)nc2n1] | 437.44 | 438.2 |
| 654 | 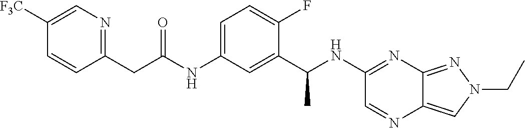 [SMILES: CCn1cc2ncc(N[C@@H](C)c3cc(NC(=O)Cc4ccc(C(F)(F)F)cn4)ccc3F)nc2n1] | 487.45 | 488.1 |
| 655 | 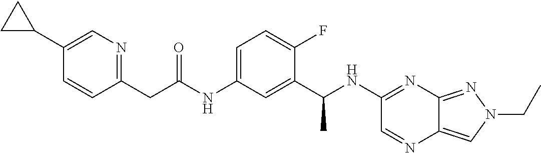 [SMILES: CCn1cc2ncc(N[C@@H](C)c3cc(NC(=O)Cc4ccc(C5CC5)cn4)ccc3F)nc2n1] | 459.52 | 460.1 |
| 656 | 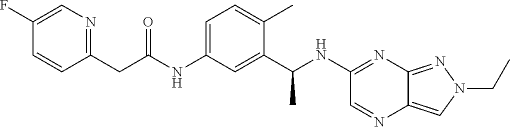 [SMILES: CCn1cc2ncc(N[C@@H](C)c3cc(NC(=O)Cc4ccc(F)cn4)ccc3C)nc2n1] | 433.48 | 434.1 |
| 657 | 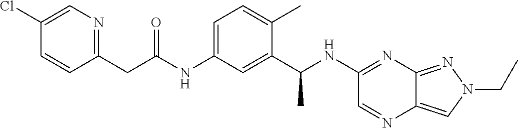 [SMILES: CCn1cc2ncc(N[C@@H](C)c3cc(NC(=O)Cc4ccc(Cl)cn4)ccc3C)nc2n1] | 449.94 | 450.1 |
| 658 | 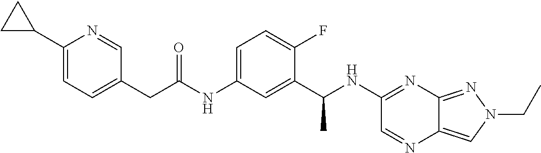 [SMILES: CCn1cc2ncc(N[C@@H](C)c3cc(NC(=O)Cc4ccc(C5CC5)nc4)ccc3F)nc2n1] | 459.52 | 460.2 |
| 659 | 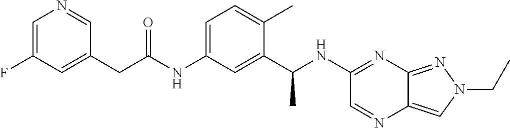 [SMILES: CCn1cc2ncc(N[C@@H](C)c3cc(NC(=O)Cc4cncc(F)c4)ccc3C)nc2n1] | 433.48 | 434.2 |
| 660 | 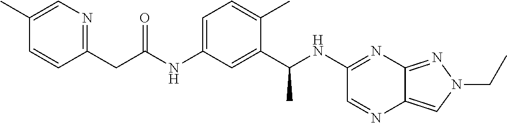 [SMILES: CCn1cc2ncc(N[C@@H](C)c3cc(NC(=O)Cc4ccc(C)cn4)ccc3C)nc2n1] | 429.52 | 430.2 |
| 661 | 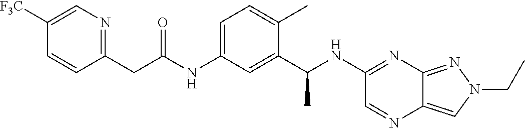 [SMILES: CCn1cc2ncc(N[C@@H](C)c3cc(NC(=O)Cc4ccc(C(F)(F)F)cn4)ccc3C)nc2n1] | 483.49 | 484.2 |
| 662 | 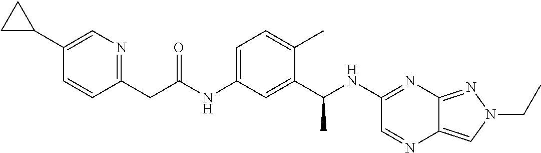 [SMILES: CCn1cc2ncc(N[C@@H](C)c3cc(NC(=O)Cc4ccc(C5CC5)cn4)ccc3C)nc2n1] | 455.55 | 456.2 |
| 663 | 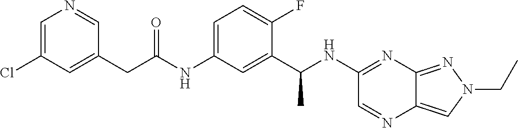 [SMILES: CCn1cc2ncc(N[C@@H](C)c3cc(NC(=O)Cc4cncc(Cl)c4)ccc3F)nc2n1] | 453.9 | 454.2 |
| 664 | 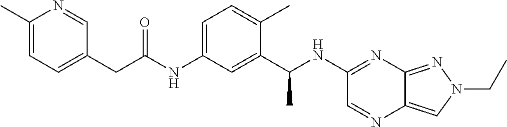 [SMILES: CCn1cc2ncc(N[C@@H](C)c3cc(NC(=O)Cc4ccc(C)nc4)ccc3C)nc2n1] | 429.52 | 430.1 |
| 665 | 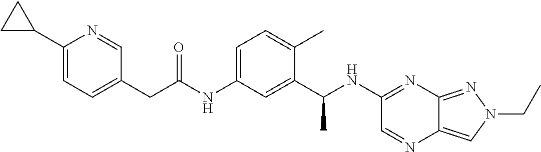 [SMILES: CCn1cc2ncc(N[C@@H](C)c3cc(NC(=O)Cc4ccc(C5CC5)nc4)ccc3C)nc2n1] | 455.55 | 455.2 |
| 666 | 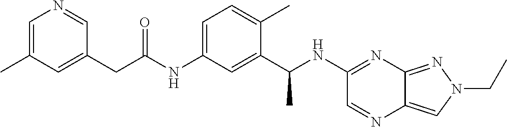 [SMILES: CCn1cc2ncc(N[C@@H](C)c3cc(NC(=O)Cc4cncc(C)c4)ccc3C)nc2n1] | 429.52 | 430.1 |
| 667 | 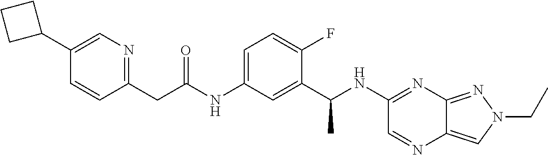 [SMILES: CCn1cc2ncc(N[C@@H](C)c3cc(NC(=O)Cc4ccc(C5CCC5)cn4)ccc3F)nc2n1] | 473.54 | 474.5 |
| 668 | 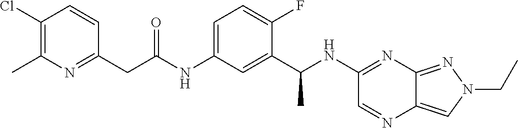 [SMILES: CCn1cc2ncc(N[C@@H](C)c3cc(NC(=O)Cc4ccc(Cl)c(C)n4)ccc3F)nc2n1] | 467.93 | 468.1 |
| 669 | 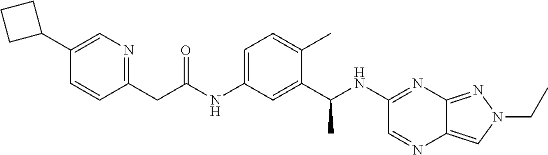 [SMILES: CCn1cc2ncc(N[C@@H](C)c3cc(NC(=O)Cc4ccc(C5CCC5)cn4)ccc3C)nc2n1] | 469.58 | 470.3 |
| 670 | 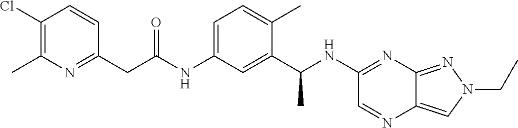 [SMILES: CCn1cc2ncc(N[C@@H](C)c3cc(NC(=O)Cc4ccc(Cl)c(C)n4)ccc3C)nc2n1] | 463.96 | 464.1 |
| 671 | 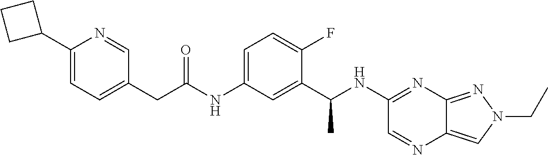 [SMILES: CCn1cc2ncc(N[C@@H](C)c3cc(NC(=O)Cc4ccc(C5CCC5)nc4)ccc3F)nc2n1] | 473.54 | 474.3 |
| 672 | 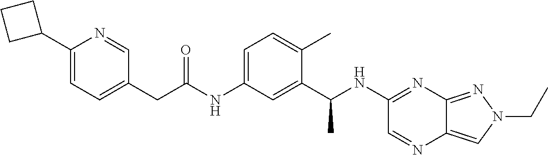 [SMILES: CCn1cc2ncc(N[C@@H](C)c3cc(NC(=O)Cc4ccc(C5CCC5)nc4)ccc3C)nc2n1] | 469.58 | 470.1 |
| 673 | 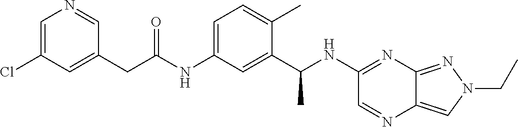 [SMILES: CCn1cc2ncc(N[C@@H](C)c3cc(NC(=O)Cc4cncc(Cl)c4)ccc3C)nc2n1] | 449.94 | 450.2 |
| 674 | 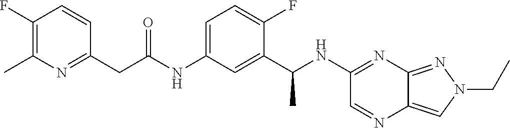 [SMILES: CCn1cc2ncc(N[C@@H](C)c3cc(NC(=O)Cc4ccc(F)c(C)n4)ccc3F)nc2n1] | 451.47 | 452.1 |
| 675 | 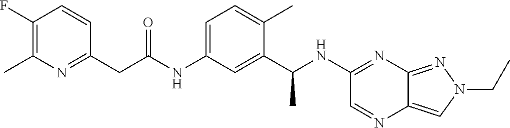 [SMILES: CCn1cc2ncc(N[C@@H](C)c3cc(NC(=O)Cc4ccc(F)c(C)n4)ccc3C)nc2n1] | 447.51 | 448.1 |
| 676 | 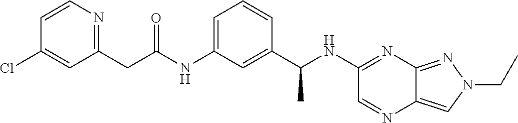 [SMILES: CCn1cc2ncc(N[C@@H](C)c3cccc(NC(=O)Cc4cc(Cl)ccn4)c3)nc2n1] | 435.91 | 436.2 |
| 677 | 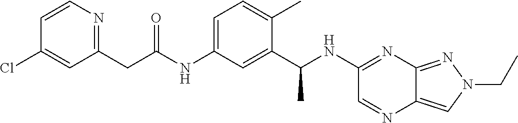 [SMILES: CCn1cc2ncc(N[C@@H](C)c3cc(NC(=O)Cc4cc(Cl)ccn4)ccc3C)nc2n1] | 449.94 | 450.2 |
| 678 | 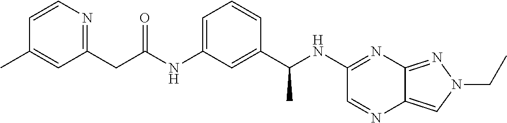 [SMILES: CCn1cc2ncc(N[C@@H](C)c3cccc(NC(=O)Cc4cc(C)ccn4)c3)nc2n1] | 415.49 | 416.1 |
| 679 | 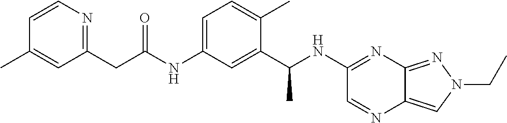 [SMILES: CCn1cc2ncc(N[C@@H](C)c3cc(NC(=O)Cc4cc(C)ccn4)ccc3C)nc2n1] | 429.52 | 430.1 |
| 680 | 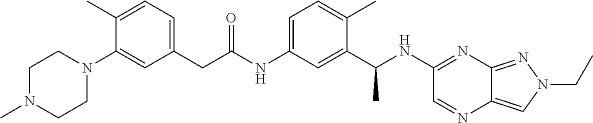 [SMILES: CCn1cc2ncc(N[C@@H](C)c3cc(NC(=O)Cc4ccc(C)c(N5CCN(C)CC5)c4)ccc3C)nc2n1] | 526.67 | 527.3 |
| 681 | 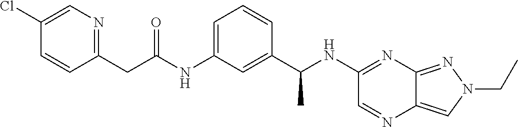 [SMILES: CCn1cc2ncc(N[C@@H](C)c3cccc(NC(=O)Cc4ccc(Cl)cn4)c3)nc2n1] | 435.91 | 436.1 |
| 682 | 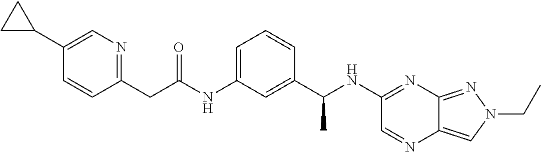 [SMILES: CCn1cc2ncc(N[C@@H](C)c3cccc(NC(=O)Cc4ccc(C5CC5)cn4)c3)nc2n1] | 441.53 | 442.3 |
| 683 | 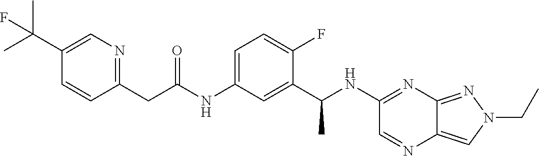 [SMILES: CCn1cc2ncc(N[C@@H](C)c3cc(NC(=O)Cc4ccc(C(C)(C)F)cn4)ccc3F)nc2n1] | 479.52 | 480.3 |
| 684 | 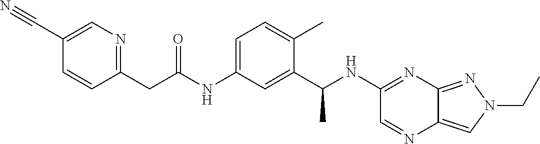 [SMILES: CCn1cc2ncc(N[C@@H](C)c3cc(NC(=O)Cc4ccc(C#N)cn4)ccc3C)nc2n1] | 440.5 | 441.1 |
| 685 | 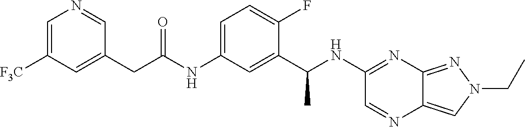 [SMILES: CCn1cc2ncc(N[C@@H](C)c3cc(NC(=O)Cc4cncc(C(F)(F)F)c4)ccc3F)nc2n1] | 487.45 | 488.2 |
| 686 | 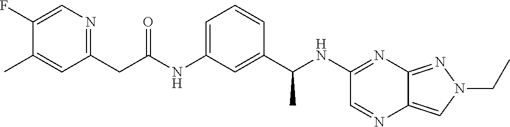 [SMILES: CCn1cc2ncc(N[C@@H](C)c3cccc(NC(=O)Cc4cc(C)c(F)cn4)c3)nc2n1] | 433.48 | 434.3 |
| 687 | 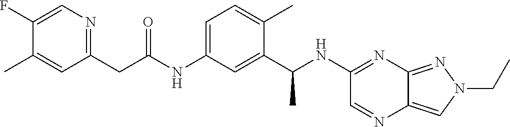 [SMILES: CCn1cc2ncc(N[C@@H](C)c3cc(NC(=O)Cc4cc(C)c(F)cn4)ccc3C)nc2n1] | 447.51 | 448.2 |
| 688 | 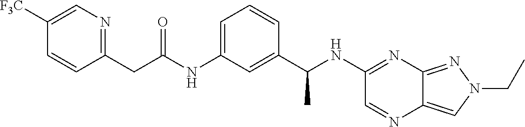 [SMILES: CCn1cc2ncc(N[C@@H](C)c3cccc(NC(=O)Cc4ccc(C(F)(F)F)cn4)c3)nc2n1] | 469.46 | 470.1 |
| 689 | 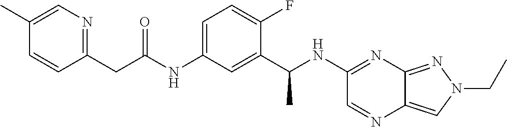 [SMILES: CCn1cc2ncc(N[C@@H](C)c3cc(NC(=O)Cc4ccc(C)cn4)ccc3F)nc2n1] | 433.48 | 434.3 |
| 690 | 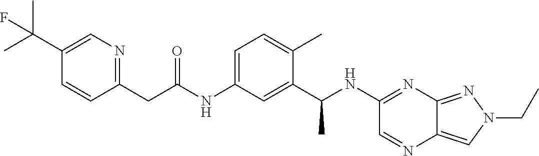 [SMILES: CCn1cc2ncc(N[C@@H](C)c3cc(NC(=O)Cc4ccc(C(C)(C)F)cn4)ccc3C)nc2n1] | 475.56 | 476.4 |
| 691 | 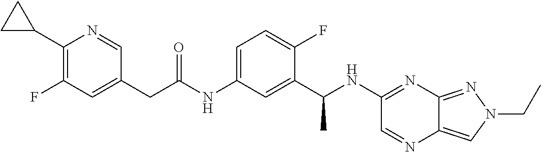 [SMILES: CCn1cc2ncc(N[C@@H](C)c3cc(NC(=O)Cc4cnc(C5CC5)c(F)c4)ccc3F)nc2n1] | 477.51 | 478.2 |
| 692 | 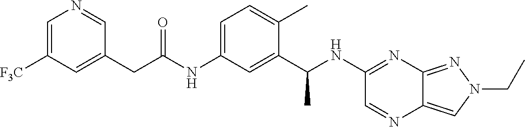 [SMILES: CCn1cc2ncc(N[C@@H](C)c3cc(NC(=O)Cc4cncc(C(F)(F)F)c4)ccc3C)nc2n1] | 483.49 | 484.1 |
| 693 | 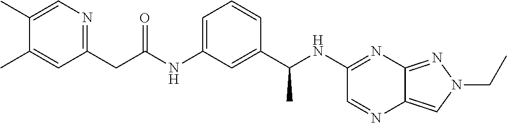 [SMILES: CCn1cc2ncc(N[C@@H](C)c3cccc(NC(=O)Cc4cc(C)c(C)cn4)c3)nc2n1] | 429.52 | 430.1 |
| 694 | 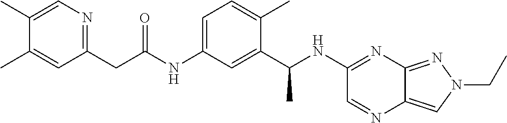 [SMILES: CCn1cc2ncc(N[C@@H](C)c3cc(NC(=O)Cc4cc(C)c(C)cn4)ccc3C)nc2n1] | 443.54 | 444.1 |
| 695 | 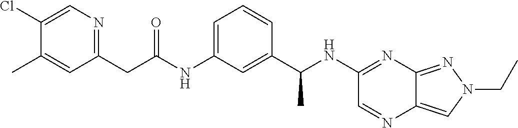 [SMILES: CCn1cc2ncc(N[C@@H](C)c3cccc(NC(=O)Cc4cc(C)c(Cl)cn4)c3)nc2n1] | 449.94 | 450.3 |
| 696 | 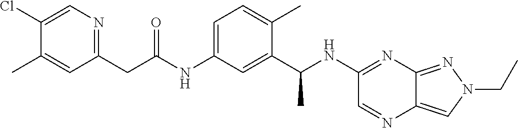 [SMILES: CCn1cc2ncc(N[C@@H](C)c3cc(NC(=O)Cc4cc(C)c(Cl)cn4)ccc3C)nc2n1] | 463.96 | 464.2 |
| 697 | 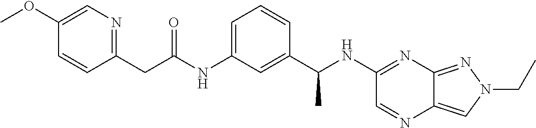 [SMILES: CCn1cc2ncc(N[C@@H](C)c3cccc(NC(=O)Cc4ccc(OC)cn4)c3)nc2n1] | 431.49 | 432.1 |
| 698 | 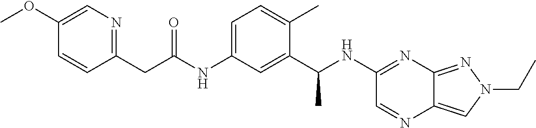 [SMILES: CCn1cc2ncc(N[C@@H](C)c3cc(NC(=O)Cc4ccc(OC)cn4)ccc3C)nc2n1] | 445.52 | 446.3 |
| 699 | 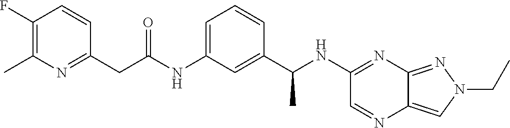 [SMILES: CCn1cc2ncc(N[C@@H](C)c3cccc(NC(=O)Cc4ccc(F)c(C)n4)c3)nc2n1] | 433.48 | 434.1 |
| 700 | 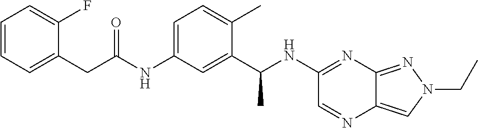 [SMILES: CCn1cc2ncc(N[C@@H](C)c3cc(NC(=O)Cc4ccccc4F)ccc3C)nc2n1] | 432.49 | 433.1 |
| 701 | 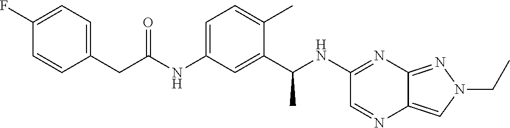 [SMILES: CCn1cc2ncc(N[C@@H](C)c3cc(NC(=O)Cc4ccc(F)cc4)ccc3C)nc2n1] | 432.49 | 433.1 |
| 702 | 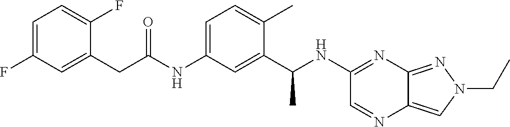 [SMILES: CCn1cc2ncc(N[C@@H](C)c3cc(NC(=O)Cc4cc(F)ccc4F)ccc3C)nc2n1] | 450.48 | 451.1 |
| 703 | 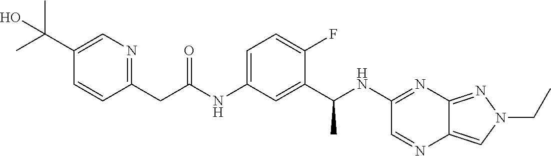 [SMILES: CCn1cc2ncc(N[C@@H](C)c3cc(NC(=O)Cc4ccc(C(C)(C)O)cn4)ccc3F)nc2n1] | 477.53 | 478.2 |
| 704 | 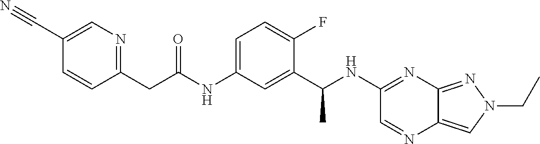 [SMILES: CCn1cc2ncc(N[C@@H](C)c3cc(NC(=O)Cc4ccc(C#N)cn4)ccc3F)nc2n1] | 444.46 | 445.1 |
| 705 | 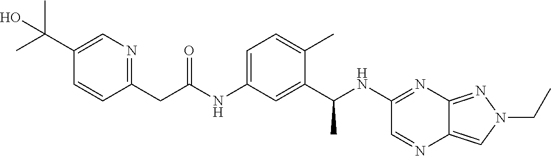 [SMILES: CCn1cc2ncc(N[C@@H](C)c3cc(NC(=O)Cc4ccc(C(C)(C)O)cn4)ccc3C)nc2n1] | 473.57 | 474.1 |
| 706 | 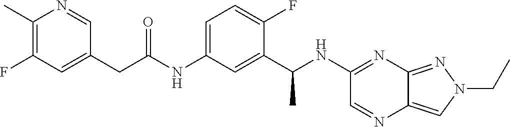 [SMILES: CCn1cc2ncc(N[C@@H](C)c3cc(NC(=O)Cc4cnc(C)c(F)c4)ccc3F)nc2n1] | 451.47 | 452.1 |
| 707 | 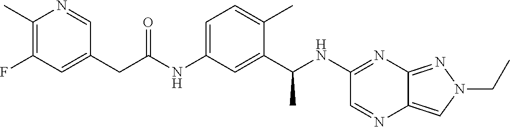 [SMILES: CCn1cc2ncc(N[C@@H](C)c3cc(NC(=O)Cc4cnc(C)c(F)c4)ccc3C)nc2n1] | 447.51 | 448.1 |
| 708 | 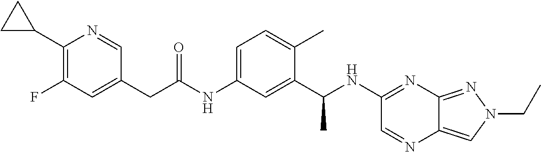 [SMILES: CCn1cc2ncc(N[C@@H](C)c3cc(NC(=O)Cc4cnc(C5CC5)c(F)c4)ccc3C)nc2n1] | 473.54 | 474.1 |
| 709 | 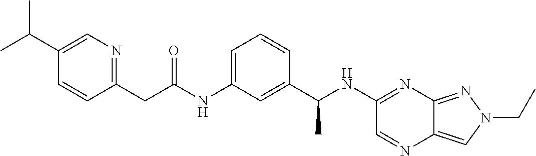 [SMILES: CCn1cc2ncc(N[C@@H](C)c3cccc(NC(=O)Cc4ccc(C(C)C)cn4)c3)nc2n1] | 443.54 | 444.1 |
| 710 | 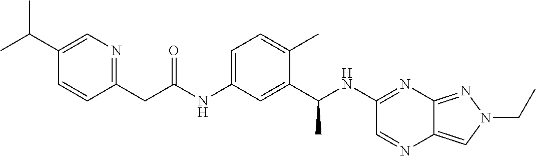 [SMILES: CCn1cc2ncc(N[C@@H](C)c3cc(NC(=O)Cc4ccc(C(C)C)cn4)ccc3C)nc2n1] | 457.57 | 458.1 |
| 711 | 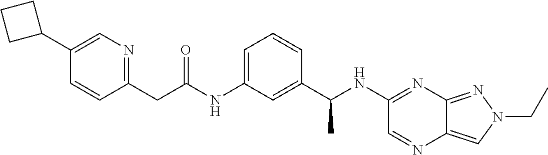 [SMILES: CCn1cc2ncc(N[C@@H](C)c3cccc(NC(=O)Cc4ccc(C5CCC5)cn4)c3)nc2n1] | 455.55 | 456.2 |
| 712 | 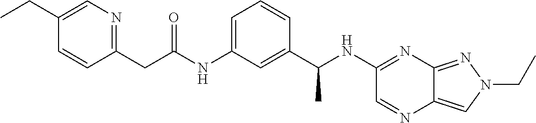 [SMILES: CCc1ccc(CC(=O)Nc2cccc([C@H](C)Nc3cnc4cn(CC)nc4n3)c2)nc1] | 429.52 | 430.1 |
| 713 | 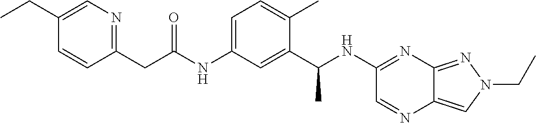 [SMILES: CCc1ccc(CC(=O)Nc2ccc(C)c([C@H](C)Nc3cnc4cn(CC)nc4n3)c2)nc1] | 443.54 | 444.1 |
| 714 | 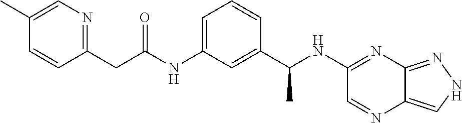 [SMILES: Cc1ccc(CC(=O)Nc2cccc([C@H](C)Nc3cnc4c[nH]nc4n3)c2)nc1] | 387.44 | 388.2 |
| 715 | 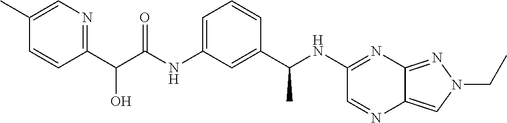 [SMILES: CCn1cc2ncc(N[C@@H](C)c3cccc(NC(=O)C(O)c4ccc(C)cn4)c3)nc2n1] | 431.49 | 432.2 |
| 716 | 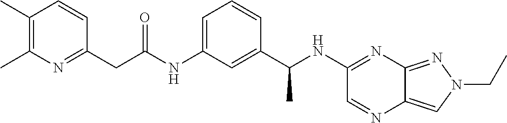 [SMILES: CCn1cc2ncc(N[C@@H](C)c3cccc(NC(=O)Cc4ccc(C)c(C)n4)c3)nc2n1] | 429.52 | 430.1 |
| 717 | 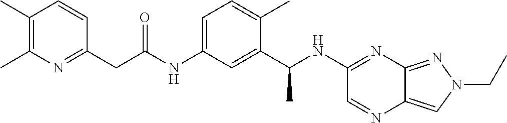 [SMILES: CCn1cc2ncc(N[C@@H](C)c3cc(NC(=O)Cc4ccc(C)c(C)n4)ccc3C)nc2n1] | 443.54 | 444.1 |
| 718 | 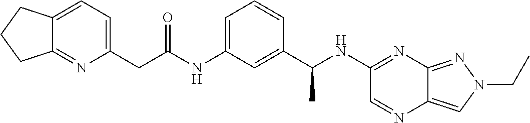 [SMILES: CCn1cc2ncc(N[C@@H](C)c3cccc(NC(=O)Cc4ccc5c(n4)CCC5)c3)nc2n1] | 441.53 | 442.2 |
| 719 | 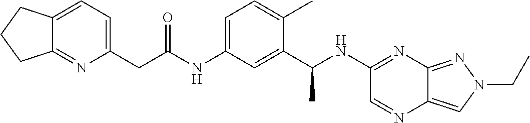 [SMILES: CCn1cc2ncc(N[C@@H](C)c3cc(NC(=O)Cc4ccc5c(n4)CCC5)ccc3C)nc2n1] | 455.55 | 456.2 |
| 720 | 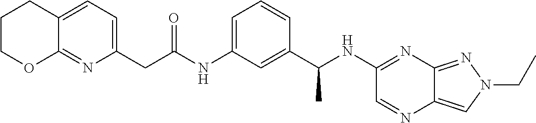 [SMILES: CCn1cc2ncc(N[C@@H](C)c3cccc(NC(=O)Cc4ccc5c(n4)OCCC5)c3)nc2n1] | 457.53 | 458.1 |
| 721 | 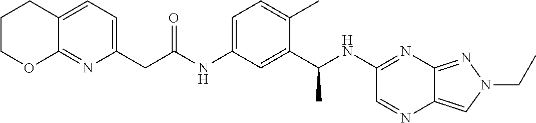 [SMILES: CCn1cc2ncc(N[C@@H](C)c3cc(NC(=O)Cc4ccc5c(n4)OCCC5)ccc3C)nc2n1] | 471.55 | 472.1 |
| 722 | 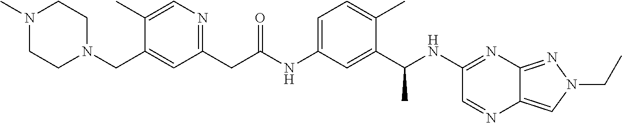 [SMILES: CCn1cc2ncc(N[C@@H](C)c3cc(NC(=O)Cc4cc(CN5CCN(C)CC5)c(C)cn4)ccc3C)nc2n1] | 541.69 | 542.4 |
| 723 | 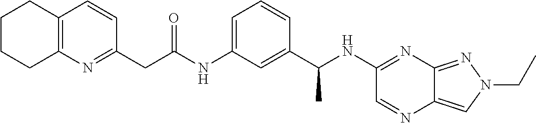 [SMILES: CCn1cc2ncc(N[C@@H](C)c3cccc(NC(=O)Cc4ccc5c(n4)CCCC5)c3)nc2n1] | 455.55 | 456.2 |
| 724 | 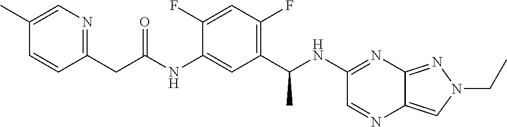 [SMILES: CCn1cc2ncc(N[C@@H](C)c3cc(NC(=O)Cc4ccc(C)cn4)c(F)cc3F)nc2n1] | 451.47 | 452.2 |
| 725 | 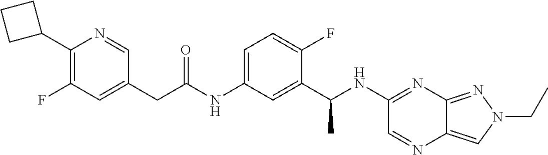 [SMILES: CCn1cc2ncc(N[C@@H](C)c3cc(NC(=O)Cc4cnc(C5CCC5)c(F)c4)ccc3F)nc2n1] | 491.54 | 492.3 |
| 726 | 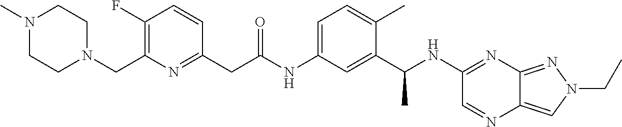 [SMILES: CCn1cc2ncc(N[C@@H](C)c3cc(NC(=O)Cc4ccc(F)c(CN5CCN(C)CC5)n4)ccc3C)nc2n1] | 545.65 | 546.2 |
| 727 | 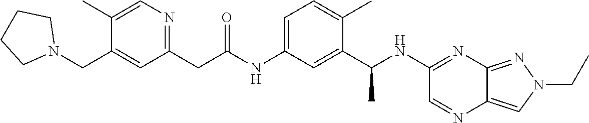 [SMILES: CCn1cc2ncc(N[C@@H](C)c3cc(NC(=O)Cc4cc(CN5CCCC5)c(C)cn4)ccc3C)nc2n1] | 512.65 | 513.4 |
| 728 | 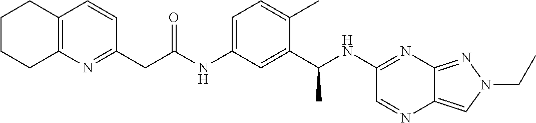 [SMILES: CCn1cc2ncc(N[C@@H](C)c3cc(NC(=O)Cc4ccc5c(n4)CCCC5)ccc3C)nc2n1] | 469.58 | 470.1 |
| 729 | 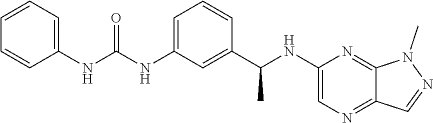 [SMILES: C[C@H](Nc1cnc2cnn(C)c2n1)c1cccc(NC(=O)Nc2ccccc2)c1] | 387.45 | 388.25 |
| 730 | 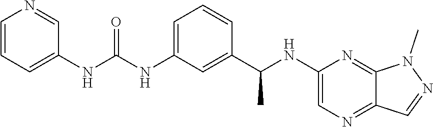 [SMILES: C[C@H](Nc1cnc2cnn(C)c2n1)c1cccc(NC(=O)Nc2cccnc2)c1] | 388.44 | 389.30 |
| 731 | 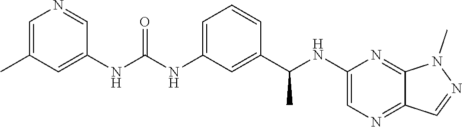 [SMILES: Cc1cncc(NC(=O)Nc2cccc([C@H](C)Nc3cnc4cnn(C)c4n3)c2)c1] | 402.46 | 403.25 |
| 732 | 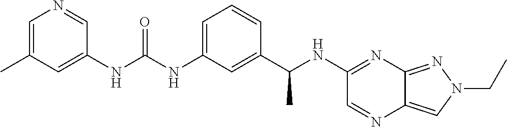 [SMILES: CCn1cc2ncc(N[C@@H](C)c3cccc(NC(=O)Nc4cncc(C)c4)c3)nc2n1] | 416.48 | 417.3 |
| 733 | 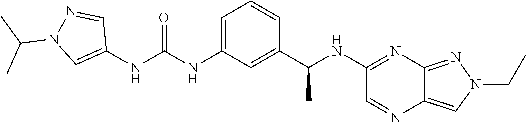 [SMILES: CCn1cc2ncc(N[C@@H](C)c3cccc(NC(=O)Nc4cnn(C(C)C)c4)c3)nc2n1] | 433.51 | 434.2 |
| 734 | 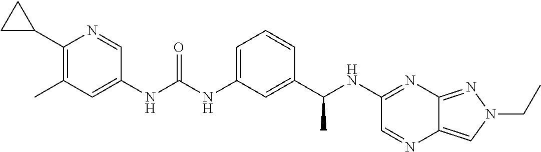 [SMILES: CCn1cc2ncc(N[C@@H](C)c3cccc(NC(=O)Nc4cnc(C5CC5)c(C)c4)c3)nc2n1] | 456.54 | 457.3 |
| 735 | 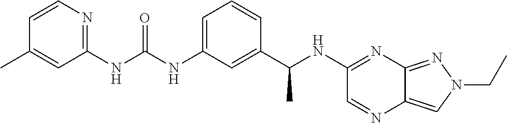 [SMILES: CCn1cc2ncc(N[C@@H](C)c3cccc(NC(=O)Nc4cc(C)ccn4)c3)nc2n1] | 416.48 | 417.2 |
| 736 | 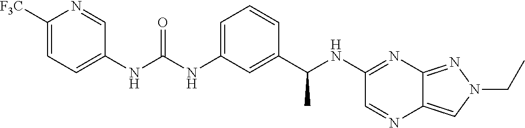 [SMILES: CCn1cc2ncc(N[C@@H](C)c3cccc(NC(=O)Nc4ccc(C(F)(F)F)nc4)c3)nc2n1] | 470.45 | 471.1 |
| 737 | 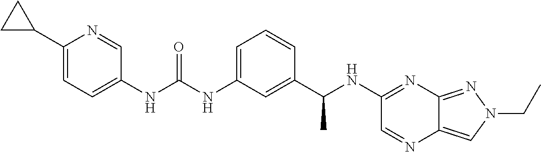 [SMILES: CCn1cc2ncc(N[C@@H](C)c3cccc(NC(=O)Nc4ccc(C5CC5)nc4)c3)nc2n1] | 442.52 | 443.2 |
| 738 | 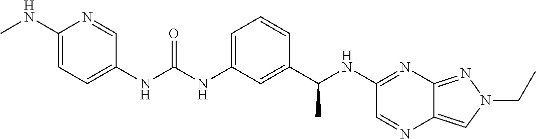 [SMILES: CCn1cc2ncc(N[C@@H](C)c3cccc(NC(=O)Nc4ccc(NC)nc4)c3)nc2n1] | 431.49 | 432.2 |
| 739 | 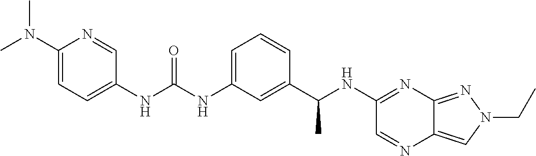 [SMILES: CCn1cc2ncc(N[C@@H](C)c3cccc(NC(=O)Nc4ccc(N(C)C)nc4)c3)nc2n1] | 445.52 | 446.3 |
| 740 | 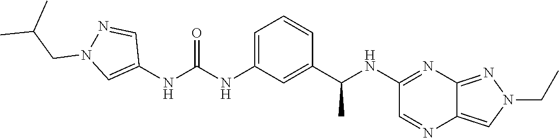 [SMILES: CCn1cc2ncc(N[C@@H](C)c3cccc(NC(=O)Nc4cnn(CC(C)C)c4)c3)nc2n1] | 447.53 | 448.3 |
| 741 | 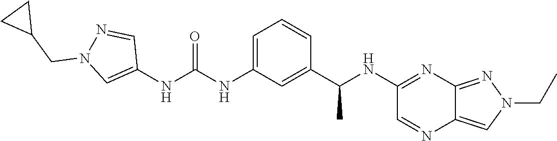 [SMILES: CCn1cc2ncc(N[C@@H](C)c3cccc(NC(=O)Nc4cnn(CC5CC5)c4)c3)nc2n1] | 445.52 | 446.1 |
| 742 | 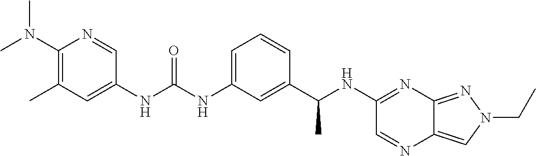 [SMILES: CCn1cc2ncc(N[C@@H](C)c3cccc(NC(=O)Nc4cnc(N(C)C)c(C)c4)c3)nc2n1] | 459.55 | 460.3 |
| 743 | 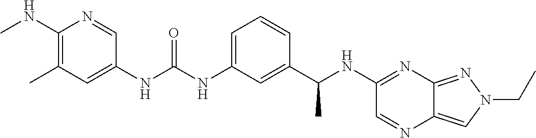 [SMILES: CCn1cc2ncc(N[C@@H](C)c3cccc(NC(=O)Nc4cnc(NC)c(C)c4)c3)nc2n1] | 445.52 | 446.3 |
| 744 | 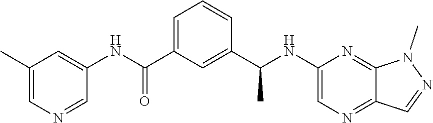 [SMILES: Cc1cncc(NC(=O)c2cccc([C@H](C)Nc3cnc4cnn(C)c4n3)c2)c1] | 387.44 | 389.46 |
| 745 | 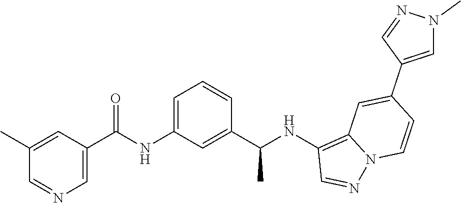 | 451.52 | 452.10 |
| 746 | 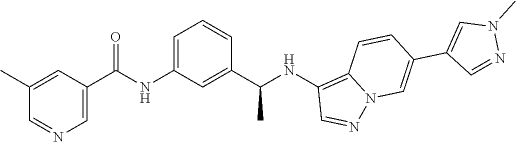 | 451.52 | 452.20 |
| 747 | 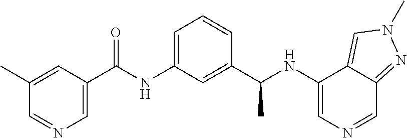 | 386.45 | 387.22 |
| 748 | 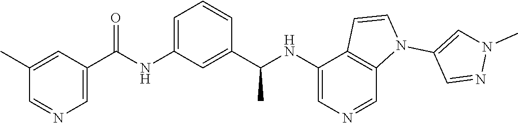 | 451.52 | 452.29 |
| 749 | 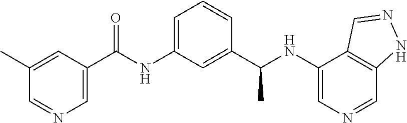 [SMILES: Cc1cncc(C(=O)Nc2cccc([C@H](C)Nc3cncc4[nH]ncc34)c2)c1] | 372.42 | 373.17 |
| 750 | 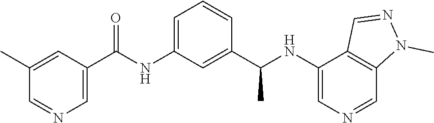 | 386.45 | 387.23 |
| 751 | 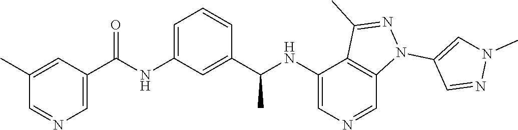 | 466.54 | 467.29 |

### Example 752

### Biological Assays

### Human PDGFRα Biochemical Inhibition Assay

The compounds described herein were tested for the ability to inhibit activity of PDGFRα which was achieved via use of an off-chip Mobility Shift Assay (MSA). Human PDGFRα, cytoplasmic domain [550-1089 (end) amino acids of accession number NP_006197.1 (SEQ ID NO: 1)] was expressed as N-terminal GST-fusion protein (89 kDa) using baculovirus expression system (SEQ ID NO: 2). GST-PDGFRα was purified by using glutathione sepharose chromatography.

Test compounds were dissolved in and diluted with dimethylsulfoxide (DMSO) to achieve 1 mM concentration. Then the solution was further diluted 25-fold with assay buffer to make the final test compound solution. The reference compound, Staurosporine, used as the assay positive control was prepared similarly. The 4× compound solution/substrate (CSKtide, 1000 nM)/ATP (Km ATP)/Mg Metal (5 mM) was prepared with kit buffer (20 mM HEPES, 0.01% Triton X-100, 5 mM DTT, pH 7.5), and 2× kinase solution was prepared with assay buffer (20 mM HEPES, 0.01% Triton X-100, 1 mM DTT, pH 7.5). The 5 μL of 4× compound solution, 5 mL of 4× Substrate/ATP/Metal solution, and 10 mL of 2× kinase solution were mixed and incubated in a well of polypropylene 384 well microplate for 1 hour at room temperature. 70 mL of Termination Buffer (QuickScout Screening Assist MSA; Carna Biosciences) was added to the well. The reaction mixture was applied to LabChip™ system (Perkin Elmer), and the product and substrate peptide peaks were separated and quantitated. The kinase reaction was evaluated by the product ratio calculated from peak heights of product(P) and substrate(S) peptides (P/(P+S)). The readout value of reaction control (complete reaction mixture) was set as a 0% inhibition, and the readout value of background (Enzyme(—)) was set as a 100% inhibition, then the percent inhibition of each test solution was calculated.

IC₅₀ values were calculated from concentration vs. % Inhibition curves by fitting to a four parameter logistic curve, and are provided for the compounds of the present invention in Table 5, below. With respect to PDGFRα activity: “+++” denotes an IC₅₀ of less than 300 nM; “++” denotes an IC₅₀ of from 300 nM to less than 1000 nM; and “+” denotes an IC₅₀ of 1000 nM or more.

### Human PDGFRβ Biochemical Inhibition Assay

The compounds described herein were tested for the ability to inhibit activity of PDGFRβ which was achieved via use of an off-chip Mobility Shift Assay (MSA). Human PDGFRβ, cytoplasmic domain [557-1106 (end) amino acids of accession number NP_002600.1 (SEQ ID NO: 3)] was expressed as N-terminal GST-fusion protein (88 kDa) using baculovirus expression system (SEQ ID NO: 4). GST-PDGFRβ was purified by using glutathione sepharose chromatography. Test compounds were dissolved in and diluted with dimethylsulfoxide (DMSO) to achieve 1 mM concentration. Then the solution was further diluted 25-fold with assay buffer to make the final test compound solution. The reference compound, Staurosporine, used as the assay positive control was prepared similarly. The 4× compound solution/substrate (CSKtide, 1000 nM)/ATP (Km ATP)/Mg Metal (5 mM) was prepared with kit buffer (20 mM HEPES, 0.01% Triton X-100, 5 mM DTT, pH 7.5), and 2× kinase solution was prepared with assay buffer (20 mM HEPES, 0.01% Triton X-100, 1 mM DTT, pH 7.5). The 5 μL of 4× compound solution, 5 mL of 4× Substrate/ATP/Metal solution, and 10 mL of 2× kinase solution were mixed and incubated in a well of polypropylene 384 well microplate for 1 hour at room temperature. 70 mL of Termination Buffer (QuickScout Screening Assist MSA; Carna Biosciences) was added to the well. The reaction mixture was applied to LabChip™ system (Perkin Elmer), and the product and substrate peptide peaks were separated and quantitated. The kinase reaction was evaluated by the product ratio calculated from peak heights of product(P) and substrate(S) peptides (P/(P+S)). The readout value of reaction control (complete reaction mixture) was set as a 0% inhibition, and the readout value of background (Enzyme(−)) was set as a 100% inhibition, then the percent inhibition of each test solution was calculated.

IC₅₀ values were calculated from concentration vs. % Inhibition curves by fitting to a four parameter logistic curve, and are provided for the compounds of the present invention in Table 7, below. With respect to PDGFRβ activity: “+++” denotes an IC₅₀ of less than 300 nM; “++” denotes an IC₅₀ of from 300 nM to less than 1000 nM; and “+” denotes an IC₅₀ of 1000 nM or more.

TABLE 7
ACTIVITY OF REPRESENTATIVE COMPOUNDS

| | PDGFRα | PDGFRβ |
|---|---|---|
| Cmpd No | IC50 | IC50 |
| 1 | +++ | +++ |
| 2 | +++ | ++ |
| 3 | +++ | + |
| 4 | +++ | +++ |
| 5 | +++ | ++ |
| 6 | +++ | + |
| 7 | +++ | + |
| 8 | +++ | +++ |
| 9 | +++ | ++ |
| 10 | +++ | +++ |
| 11 | +++ | +++ |
| 12 | +++ | +++ |
| 13 | Not Tested | Not Tested |
| 14 | +++ | +++ |
| 15 | +++ | +++ |
| 16 | +++ | +++ |
| 17 | +++ | +++ |
| 18 | +++ | +++ |
| 19 | +++ | +++ |
| 20 | + | + |
| 21 | +++ | +++ |
| 22 | +++ | +++ |
| 23 | ++ | ++ |
| 24 | ++ | ++ |
| 25 | +++ | + |
| 26 | ++ | + |
| 27 | +++ | +++ |
| 28 | +++ | +++ |
| 29 | +++ | +++ |
| 30 | +++ | ++ |
| 31 | +++ | +++ |
| 32 | +++ | +++ |
| 33 | +++ | +++ |
| 34 | +++ | ++ |
| 35 | +++ | +++ |
| 36 | +++ | +++ |
| 37 | +++ | +++ |
| 38 | +++ | +++ |
| 39 | +++ | +++ |
| 40 | +++ | +++ |
| 41 | +++ | +++ |
| 42 | +++ | +++ |
| 43 | ++ | + |
| 44 | +++ | + |
| 45 | ++ | + |
| 46 | +++ | +++ |
| 47 | +++ | +++ |
| 48 | +++ | +++ |
| 49 | +++ | +++ |
| 50 | +++ | +++ |
| 51 | +++ | +++ |
| 52 | ++ | + |
| 53 | + | + |
| 54 | +++ | +++ |
| 55 | +++ | +++ |
| 56 | +++ | +++ |
| 57 | +++ | +++ |
| 58 | +++ | +++ |
| 59 | +++ | +++ |
| 60 | + | + |
| 61 | ++ | + |
| 62 | ++ | + |
| 63 | +++ | +++ |
| 64 | +++ | ++ |
| 65 | Not Tested | Not Tested |
| 66 | +++ | ++ |
| 67 | +++ | +++ |
| 68 | +++ | +++ |
| 69 | +++ | +++ |
| 70 | +++ | +++ |
| 71 | +++ | +++ |
| 72 | +++ | +++ |
| 73 | +++ | +++ |
| 74 | +++ | +++ |
| 75 | Not Tested | Not Tested |
| 76 | +++ | +++ |
| 77 | +++ | ++ |
| 78 | Not Tested | Not Tested |
| 79 | +++ | +++ |
| 80 | ++ | + |
| 81 | + | + |
| 82 | +++ | ++ |
| 83 | +++ | + |
| 84 | +++ | ++ |
| 85 | + | + |
| 86 | +++ | +++ |
| 87 | +++ | +++ |
| 88 | +++ | + |
| 89 | +++ | ++ |
| 90 | +++ | +++ |
| 91 | +++ | +++ |
| 92 | +++ | + |
| 93 | +++ | +++ |
| 94 | +++ | +++ |
| 95 | +++ | +++ |
| 96 | +++ | +++ |
| 97 | +++ | +++ |
| 98 | +++ | +++ |
| 99 | +++ | +++ |
| 100 | +++ | +++ |
| 101 | +++ | +++ |
| 102 | +++ | +++ |
| 103 | +++ | +++ |
| 104 | +++ | +++ |
| 105 | +++ | +++ |
| 106 | +++ | ++ |
| 107 | +++ | +++ |
| 108 | +++ | +++ |
| 109 | +++ | +++ |
| 110 | Not Tested | Not Tested |
| 111 | +++ | +++ |
| 112 | +++ | +++ |
| 113 | +++ | ++ |
| 114 | +++ | +++ |
| 115 | +++ | +++ |
| 116 | +++ | +++ |
| 117 | +++ | + |
| 118 | +++ | +++ |
| 119 | +++ | +++ |
| 120 | +++ | ++ |
| 121 | +++ | +++ |
| 122 | +++ | ++ |
| 123 | +++ | +++ |
| 124 | +++ | +++ |
| 125 | +++ | +++ |
| 126 | ++ | + |
| 127 | +++ | +++ |
| 128 | +++ | +++ |
| 129 | +++ | +++ |
| 130 | +++ | +++ |
| 131 | ++ | + |
| 132 | +++ | + |
| 133 | +++ | +++ |
| 134 | +++ | +++ |
| 135 | +++ | +++ |
| 136 | +++ | +++ |
| 137 | +++ | +++ |
| 138 | +++ | +++ |
| 139 | +++ | +++ |
| 140 | +++ | +++ |
| 141 | +++ | +++ |
| 142 | +++ | +++ |
| 143 | +++ | +++ |
| 144 | +++ | +++ |
| 145 | +++ | +++ |
| 146 | ++ | + |
| 147 | +++ | +++ |
| 148 | +++ | +++ |
| 149 | +++ | +++ |
| 150 | +++ | +++ |
| 151 | +++ | +++ |
| 152 | +++ | +++ |
| 153 | +++ | +++ |
| 154 | +++ | +++ |
| 155 | +++ | + |
| 156 | +++ | +++ |
| 157 | +++ | +++ |
| 158 | +++ | ++ |
| 159 | +++ | +++ |
| 160 | +++ | +++ |
| 161 | +++ | +++ |
| 162 | +++ | +++ |
| 163 | +++ | +++ |
| 164 | +++ | +++ |
| 165 | +++ | +++ |
| 166 | +++ | +++ |
| 167 | +++ | +++ |
| 168 | +++ | + |
| 169 | +++ | +++ |
| 170 | +++ | ++ |
| 171 | +++ | +++ |
| 172 | +++ | +++ |
| 173 | Not Tested | Not Tested |
| 174 | Not Tested | Not Tested |
| 175 | Not Tested | Not Tested |
| 176 | +++ | +++ |
| 177 | +++ | +++ |
| 178 | +++ | +++ |
| 179 | ++ | + |
| 180 | ++ | + |
| 181 | +++ | +++ |
| 182 | +++ | + |
| 183 | +++ | ++ |
| 184 | +++ | +++ |
| 185 | +++ | +++ |
| 186 | +++ | +++ |
| 187 | +++ | +++ |
| 188 | +++ | +++ |
| 189 | +++ | +++ |
| 190 | +++ | +++ |
| 191 | +++ | +++ |
| 192 | +++ | +++ |
| 193 | +++ | +++ |
| 194 | +++ | +++ |
| 195 | +++ | +++ |
| 196 | +++ | +++ |
| 197 | +++ | +++ |
| 198 | +++ | +++ |
| 199 | +++ | +++ |
| 200 | +++ | +++ |
| 201 | +++ | +++ |
| 202 | +++ | +++ |
| 203 | +++ | +++ |
| 204 | +++ | +++ |
| 205 | +++ | +++ |
| 206 | +++ | +++ |
| 207 | +++ | +++ |
| 208 | +++ | +++ |
| 209 | +++ | +++ |
| 210 | +++ | +++ |
| 211 | +++ | +++ |
| 212 | ++ | + |
| 213 | +++ | +++ |
| 214 | +++ | +++ |
| 215 | +++ | +++ |
| 216 | +++ | +++ |
| 217 | Not Tested | Not Tested |
| 218 | +++ | +++ |
| 219 | +++ | +++ |
| 220 | +++ | ++ |
| 221 | +++ | +++ |
| 222 | +++ | +++ |
| 223 | +++ | +++ |
| 224 | +++ | +++ |
| 225 | +++ | +++ |
| 226 | +++ | +++ |
| 227 | +++ | +++ |
| 228 | +++ | +++ |
| 229 | +++ | +++ |
| 230 | +++ | +++ |
| 231 | +++ | +++ |
| 232 | +++ | +++ |
| 233 | +++ | +++ |
| 234 | +++ | +++ |
| 235 | Not Tested | Not Tested |
| 236 | +++ | +++ |
| 237 | +++ | +++ |
| 238 | +++ | +++ |
| 239 | +++ | +++ |
| 240 | +++ | +++ |
| 241 | +++ | ++ |
| 242 | +++ | +++ |
| 243 | +++ | +++ |
| 244 | +++ | +++ |
| 245 | +++ | +++ |
| 246 | +++ | +++ |
| 247 | +++ | +++ |
| 248 | +++ | +++ |
| 249 | +++ | +++ |
| 250 | +++ | +++ |
| 251 | +++ | +++ |
| 252 | +++ | +++ |
| 253 | +++ | + |
| 254 | +++ | +++ |
| 255 | +++ | +++ |
| 256 | +++ | +++ |
| 257 | +++ | +++ |
| 258 | +++ | +++ |
| 259 | +++ | +++ |
| 260 | +++ | +++ |
| 261 | +++ | +++ |
| 262 | +++ | +++ |
| 263 | +++ | ++ |
| 264 | +++ | +++ |
| 265 | +++ | +++ |
| 266 | +++ | +++ |
| 267 | +++ | +++ |
| 268 | +++ | +++ |
| 269 | +++ | +++ |
| 270 | Not Tested | Not Tested |
| 271 | +++ | +++ |
| 272 | +++ | +++ |
| 273 | +++ | +++ |
| 274 | +++ | + |
| 275 | +++ | +++ |
| 276 | ++ | ++ |
| 277 | +++ | +++ |
| 278 | ++ | + |
| 279 | +++ | ++ |
| 280 | +++ | +++ |
| 281 | +++ | ++ |
| 282 | +++ | ++ |
| 283 | +++ | +++ |
| 284 | ++ | ++ |
| 285 | ++ | + |
| 286 | +++ | ++ |
| 287 | +++ | +++ |
| 288 | +++ | +++ |
| 289 | +++ | ++ |
| 290 | +++ | +++ |
| 291 | +++ | +++ |
| 292 | +++ | +++ |
| 293 | +++ | +++ |
| 294 | ++ | ++ |
| 295 | +++ | +++ |
| 296 | +++ | +++ |
| 297 | ++ | ++ |
| 298 | +++ | ++ |
| 299 | +++ | ++ |
| 300 | +++ | +++ |
| 301 | +++ | +++ |
| 302 | +++ | +++ |
| 303 | +++ | +++ |
| 304 | +++ | +++ |
| 305 | +++ | ++ |
| 306 | +++ | +++ |
| 307 | +++ | +++ |
| 308 | +++ | +++ |
| 309 | +++ | +++ |
| 310 | +++ | +++ |
| 311 | +++ | +++ |
| 312 | +++ | +++ |
| 313 | +++ | +++ |
| 314 | +++ | +++ |
| 315 | +++ | +++ |
| 316 | +++ | +++ |
| 317 | +++ | +++ |
| 318 | +++ | +++ |
| 319 | +++ | +++ |
| 320 | +++ | +++ |
| 321 | +++ | +++ |
| 322 | +++ | +++ |
| 323 | +++ | +++ |
| 324 | +++ | ++ |
| 325 | + | + |
| 326 | +++ | +++ |
| 327 | +++ | +++ |
| 328 | +++ | +++ |
| 329 | +++ | +++ |
| 330 | +++ | +++ |
| 331 | +++ | +++ |
| 332 | +++ | +++ |
| 333 | +++ | ++ |
| 334 | +++ | +++ |
| 335 | +++ | +++ |
| 336 | +++ | +++ |
| 337 | +++ | +++ |
| 338 | +++ | ++ |
| 339 | +++ | ++ |
| 340 | +++ | +++ |
| 341 | +++ | +++ |
| 342 | ++ | + |
| 343 | +++ | +++ |
| 344 | +++ | +++ |
| 345 | +++ | +++ |
| 346 | ++ | ++ |
| 347 | +++ | +++ |
| 348 | +++ | +++ |
| 349 | +++ | +++ |
| 350 | +++ | +++ |
| 351 | ++ | + |
| 352 | +++ | ++ |
| 353 | +++ | +++ |
| 354 | +++ | +++ |
| 355 | ++ | + |
| 356 | +++ | +++ |
| 357 | +++ | +++ |
| 358 | +++ | +++ |
| 359 | +++ | +++ |
| 360 | +++ | +++ |
| 361 | +++ | ++ |
| 362 | +++ | ++ |
| 363 | + | + |
| 364 | +++ | ++ |
| 365 | +++ | +++ |
| 366 | +++ | +++ |
| 367 | ++ | + |
| 368 | + | + |
| 369 | + | + |
| 370 | ++ | + |
| 371 | +++ | ++ |
| 372 | ++ | + |
| 373 | +++ | + |
| 374 | ++ | ++ |
| 375 | +++ | ++ |
| 376 | +++ | +++ |
| 377 | ++ | ++ |
| 378 | +++ | ++ |
| 379 | +++ | ++ |
| 380 | +++ | ++ |
| 381 | +++ | ++ |
| 382 | Not Tested | Not Tested |
| 383 | +++ | +++ |
| 384 | +++ | +++ |
| 385 | +++ | ++ |
| 386 | +++ | +++ |
| 387 | +++ | + |
| 388 | + | + |
| 389 | +++ | +++ |
| 390 | +++ | +++ |
| 391 | +++ | +++ |
| 392 | ++ | + |
| 393 | ++ | + |
| 394 | +++ | + |
| 395 | +++ | ++ |
| 396 | +++ | +++ |
| 397 | ++ | + |
| 398 | +++ | +++ |
| 399 | +++ | +++ |
| 400 | ++ | + |
| 401 | +++ | +++ |
| 402 | +++ | +++ |
| 403 | +++ | +++ |
| 404 | +++ | +++ |
| 405 | +++ | +++ |
| 406 | Not Tested | Not Tested |
| 407 | +++ | +++ |
| 408 | ++ | + |
| 409 | Not Tested | Not Tested |
| 410 | +++ | +++ |
| 411 | +++ | +++ |
| 412 | +++ | +++ |
| 413 | +++ | +++ |
| 414 | +++ | +++ |
| 415 | +++ | +++ |
| 416 | +++ | +++ |
| 417 | +++ | +++ |
| 418 | +++ | +++ |
| 419 | +++ | ++ |
| 420 | +++ | +++ |
| 421 | +++ | +++ |
| 422 | +++ | +++ |
| 423 | +++ | +++ |
| 424 | +++ | +++ |
| 425 | +++ | +++ |
| 426 | +++ | +++ |
| 427 | +++ | +++ |
| 428 | +++ | +++ |
| 429 | +++ | +++ |
| 430 | +++ | +++ |
| 431 | +++ | +++ |
| 432 | +++ | +++ |
| 433 | +++ | +++ |
| 434 | +++ | +++ |
| 435 | +++ | +++ |
| 436 | +++ | +++ |
| 437 | +++ | +++ |
| 438 | +++ | +++ |
| 439 | +++ | +++ |
| 440 | +++ | +++ |
| 441 | +++ | +++ |
| 442 | +++ | +++ |
| 443 | +++ | +++ |
| 444 | +++ | +++ |
| 445 | +++ | +++ |
| 446 | +++ | +++ |
| 447 | +++ | +++ |
| 448 | Not Tested | Not Tested |
| 449 | ++ | + |
| 450 | +++ | +++ |
| 451 | +++ | ++ |
| 452 | +++ | +++ |
| 453 | +++ | +++ |
| 454 | +++ | ++ |
| 455 | +++ | +++ |
| 456 | +++ | +++ |
| 457 | +++ | +++ |
| 458 | +++ | +++ |
| 459 | + | + |
| 460 | +++ | +++ |
| 461 | +++ | ++ |
| 462 | +++ | +++ |
| 463 | +++ | +++ |
| 464 | +++ | +++ |
| 465 | +++ | + |
| 466 | +++ | ++ |
| 467 | +++ | + |
| 468 | ++ | + |
| 469 | +++ | +++ |
| 470 | +++ | +++ |
| 471 | ++ | + |
| 472 | +++ | ++ |
| 473 | +++ | +++ |
| 474 | +++ | +++ |
| 475 | +++ | +++ |
| 476 | +++ | ++ |
| 477 | +++ | +++ |
| 478 | +++ | ++ |
| 479 | +++ | +++ |
| 480 | +++ | ++ |
| 481 | +++ | +++ |
| 482 | +++ | ++ |
| 483 | +++ | +++ |
| 484 | +++ | +++ |
| 485 | Not Tested | Not Tested |
| 486 | +++ | +++ |
| 487 | +++ | +++ |
| 488 | +++ | +++ |
| 489 | +++ | +++ |
| 490 | +++ | +++ |
| 491 | ++ | ++ |
| 492 | +++ | ++ |
| 493 | +++ | +++ |
| 494 | +++ | +++ |
| 495 | +++ | +++ |
| 496 | +++ | ++ |
| 497 | +++ | +++ |
| 498 | +++ | +++ |
| 499 | +++ | +++ |
| 500 | + | + |
| 501 | +++ | +++ |
| 502 | +++ | +++ |
| 503 | ++ | ++ |
| 504 | +++ | +++ |
| 505 | +++ | +++ |
| 506 | +++ | +++ |
| 507 | +++ | +++ |
| 508 | +++ | +++ |
| 509 | +++ | +++ |
| 510 | +++ | +++ |
| 511 | +++ | +++ |
| 512 | +++ | +++ |
| 513 | +++ | +++ |
| 514 | +++ | +++ |
| 515 | +++ | +++ |
| 516 | + | + |
| 517 | Not Tested | Not Tested |
| 518 | +++ | +++ |
| 519 | Not Tested | Not Tested |
| 520 | +++ | +++ |
| 521 | +++ | +++ |
| 522 | +++ | +++ |
| 523 | +++ | +++ |
| 524 | +++ | +++ |
| 525 | Not Tested | Not Tested |
| 526 | +++ | +++ |
| 527 | +++ | +++ |
| 528 | +++ | +++ |
| 529 | +++ | +++ |
| 530 | +++ | +++ |
| 531 | +++ | +++ |
| 532 | +++ | +++ |
| 533 | +++ | +++ |
| 534 | +++ | +++ |
| 535 | +++ | +++ |
| 536 | +++ | +++ |
| 537 | +++ | +++ |
| 538 | +++ | +++ |
| 539 | +++ | +++ |
| 540 | +++ | +++ |
| 541 | +++ | +++ |
| 542 | +++ | +++ |
| 543 | +++ | +++ |
| 544 | +++ | +++ |
| 545 | +++ | +++ |
| 546 | +++ | +++ |
| 547 | +++ | +++ |
| 548 | +++ | +++ |
| 549 | +++ | +++ |
| 550 | +++ | +++ |
| 551 | +++ | +++ |
| 552 | +++ | +++ |
| 553 | +++ | +++ |
| 554 | +++ | +++ |
| 555 | +++ | +++ |
| 556 | +++ | +++ |
| 557 | +++ | +++ |
| 558 | +++ | +++ |
| 559 | +++ | +++ |
| 560 | +++ | +++ |
| 561 | +++ | +++ |
| 562 | +++ | ++ |
| 563 | +++ | +++ |
| 564 | +++ | +++ |
| 565 | +++ | +++ |
| 566 | +++ | +++ |
| 567 | +++ | ++ |
| 568 | ++ | + |
| 569 | ++ | +++ |
| 570 | +++ | +++ |
| 571 | +++ | +++ |
| 572 | + | ++ |
| 573 | +++ | +++ |
| 574 | +++ | +++ |
| 575 | +++ | +++ |
| 576 | +++ | +++ |
| 577 | +++ | +++ |
| 578 | +++ | +++ |
| 579 | +++ | +++ |
| 580 | ++ | +++ |
| 581 | +++ | +++ |
| 582 | ++ | ++ |
| 583 | +++ | +++ |
| 584 | ++ | ++ |
| 585 | +++ | +++ |
| 586 | +++ | +++ |
| 587 | +++ | +++ |
| 588 | +++ | +++ |
| 589 | + | ++ |
| 590 | Not Tested | Not Tested |
| 591 | +++ | +++ |
| 592 | +++ | +++ |
| 593 | +++ | +++ |
| 594 | +++ | +++ |
| 595 | +++ | +++ |
| 596 | +++ | +++ |
| 597 | +++ | +++ |
| 598 | +++ | +++ |
| 599 | +++ | +++ |
| 600 | +++ | ++ |
| 601 | +++ | +++ |
| 602 | +++ | +++ |
| 603 | +++ | +++ |
| 604 | +++ | ++ |
| 605 | +++ | +++ |
| 606 | +++ | +++ |
| 607 | +++ | +++ |
| 608 | +++ | +++ |
| 609 | +++ | +++ |
| 610 | +++ | +++ |
| 611 | +++ | +++ |
| 612 | +++ | +++ |
| 613 | +++ | +++ |
| 614 | +++ | +++ |
| 615 | +++ | +++ |
| 616 | +++ | +++ |
| 617 | +++ | +++ |
| 618 | +++ | +++ |
| 619 | +++ | +++ |
| 620 | +++ | +++ |
| 621 | +++ | +++ |
| 622 | +++ | +++ |
| 623 | ++ | + |
| 624 | ++ | + |
| 625 | +++ | ++ |
| 626 | +++ | + |
| 627 | +++ | ++ |
| 628 | +++ | + |
| 629 | +++ | + |
| 630 | +++ | ++ |
| 631 | +++ | +++ |
| 632 | +++ | +++ |
| 633 | +++ | ++ |
| 634 | +++ | +++ |
| 635 | +++ | +++ |
| 636 | +++ | +++ |
| 637 | +++ | +++ |
| 638 | +++ | +++ |
| 639 | +++ | +++ |
| 640 | +++ | ++ |
| 641 | +++ | +++ |
| 642 | +++ | ++ |
| 643 | +++ | ++ |
| 644 | + | + |
| 645 | ++ | + |
| 646 | +++ | ++ |
| 647 | +++ | + |
| 648 | +++ | +++ |
| 649 | +++ | ++ |
| 650 | +++ | ++ |
| 651 | +++ | + |
| 652 | +++ | ++ |
| 653 | +++ | + |
| 654 | +++ | + |
| 655 | +++ | + |
| 656 | +++ | +++ |
| 657 | +++ | +++ |
| 658 | ++ | + |
| 659 | +++ | +++ |
| 660 | +++ | ++ |
| 661 | +++ | ++ |
| 662 | +++ | + |
| 663 | +++ | ++ |
| 664 | +++ | + |
| 665 | +++ | + |
| 666 | +++ | +++ |
| 667 | +++ | + |
| 668 | +++ | +++ |
| 669 | +++ | ++ |
| 670 | +++ | +++ |
| 671 | +++ | + |
| 672 | +++ | + |
| 673 | +++ | +++ |
| 674 | +++ | +++ |
| 675 | +++ | +++ |
| 676 | ++ | + |
| 677 | +++ | +++ |
| 678 | +++ | + |
| 679 | +++ | +++ |
| 680 | +++ | +++ |
| 681 | +++ | + |
| 682 | ++ | + |
| 683 | ++ | + |
| 684 | Not Tested | Not Tested |
| 685 | +++ | ++ |
| 686 | +++ | ++ |
| 687 | +++ | +++ |
| 688 | +++ | + |
| 689 | +++ | + |
| 690 | ++ | + |
| 691 | +++ | + |
| 692 | +++ | +++ |
| 693 | +++ | + |
| 694 | +++ | +++ |
| 695 | +++ | ++ |
| 696 | +++ | +++ |
| 697 | ++ | + |
| 698 | +++ | ++ |
| 699 | +++ | ++ |
| 700 | +++ | +++ |
| 701 | +++ | +++ |
| 702 | +++ | +++ |
| 703 | + | + |
| 704 | Not Tested | Not Tested |
| 705 | + | + |
| 706 | +++ | + |
| 707 | +++ | ++ |
| 708 | +++ | ++ |
| 709 | ++ | + |
| 710 | +++ | + |
| 711 | +++ | + |
| 712 | +++ | + |
| 713 | +++ | ++ |
| 714 | Not Tested | Not Tested |
| 715 | Not Tested | Not Tested |
| 716 | +++ | + |
| 717 | +++ | +++ |
| 718 | +++ | + |
| 719 | +++ | +++ |
| 720 | +++ | + |
| 721 | +++ | + |
| 722 | +++ | + |
| 723 | +++ | + |
| 724 | +++ | + |
| 725 | Not Tested | Not Tested |
| 726 | +++ | ++ |
| 727 | + | + |
| 728 | + | + |
| 729 | +++ | +++ |
| 730 | +++ | ++ |
| 731 | +++ | +++ |
| 732 | +++ | ++ |
| 733 | +++ | ++ |
| 734 | +++ | +++ |
| 735 | +++ | +++ |
| 736 | +++ | +++ |
| 737 | +++ | +++ |
| 738 | ++ | + |
| 739 | +++ | + |
| 740 | +++ | ++ |
| 741 | +++ | + |
| 742 | +++ | +++ |
| 743 | +++ | ++ |
| 744 | +++ | + |
| 745 | + | + |
| 746 | +++ | +++ |
| 747 | + | + |
| 748 | +++ | ++ |
| 749 | + | + |
| 750 | + | + |
| 751 | + | + |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

This application claims the benefit of priority to U.S. Provisional Application No. 62/868,735, filed Jun. 28, 2019, which application is hereby incorporated by reference in its entirety.

1. A compound having the structure of Formula (I): 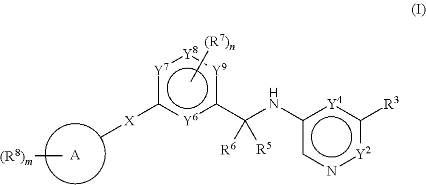
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
ring A is carbocycle or heterocycle;
Y² is a bond, —CR²═, NR², or —N═;
Y⁴ is a bond, —CR⁴═, —NR⁴—, or —N═;
R² or R⁴, together with R³ and the atoms to which they are attached, form ring B;
R² is H, halogen, cyano, alkyl, haloalkyl, alkoxy, —NH₂, —NHR⁹, or —NR⁹R⁹ when R³ and R⁴ form ring B;
R⁴ is H, halogen, cyano, alkyl, haloalkyl, alkoxy, —NH₂, —NHR⁹, or —NR⁹R⁹ when R³ and R² form ring B;
ring B is a 5- to 6-membered carbocycle or 5- to 6-membered heterocycle, wherein ring B is substituted by (R⁹)ₚ;
Y⁶, Y⁷, Y⁸, and Y⁹ are each, independently, —CH═, —CR⁷═, or N;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
R¹⁰ is H, alkyl, or haloalkyl;
R¹¹ is H, alkyl, or haloalkyl;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when ring B is a 6-membered carbocycle;
n is 0-5;
p is 0-5; and
q is 0-2.

2. The compound of claim 1, or a pharmaceutically acceptable isomer, racemate, hydrate, solvate, tautomer, isotope, or salt thereof, wherein ring A is a monocyclic carbocycle.

3. The compound of claim 1, or a pharmaceutically acceptable isomer, racemate, hydrate, solvate, tautomer, isotope, or salt thereof, wherein ring A is a polycyclic carbocycle.

4. The compound of claim 1, or a pharmaceutically acceptable isomer, racemate, hydrate, solvate, tautomer, isotope, or salt thereof, wherein ring A is a monocyclic heterocycle.

5. The compound of claim 4, or a pharmaceutically acceptable isomer, racemate, hydrate, solvate, tautomer, isotope, or salt thereof, wherein ring A is pyrrolidinyl, pyrrolyl, pyrazolyl, imidazolyl, triazolyl, tetrazolyl, furanyl, oxazolyl, isoxazolyl, oxadiazolyl, oxatriazolyl, thiophenyl, thiazolyl, isothiazolyl, thiadiazolyl, thiatriazolyl, piperidinyl, piperazinyl, pyridinyl, pyrazinyl, pyrimidinyl, pyridazinyl, triazinyl, 2H-pyranyl, tetrahydro-2H-pyranyl, or morpholinyl.

6. The compound of claim 1, or a pharmaceutically acceptable isomer, racemate, hydrate, solvate, tautomer, isotope, or salt thereof, wherein ring A is a polycyclic heterocycle.

7. The compound of claim 6, or a pharmaceutically acceptable isomer, racemate, hydrate, solvate, tautomer, isotope, or salt thereof, wherein ring A is indolyl, benzimidazolyl, indazolyl, benzotriazolyl, 1H-pyrrolo[3,2-b]pyridinyl, 1H-pyrrolo[3,2-c]pyridinyl, pyrrolo[2,3-b]pyridinyl, pyrrolo[2,3-c]pyridinyl, [1,2,3]triazolo[4,5-b]pyridinyl, 7H-pyrrolo[2,3-d]pyrimidinyl, 5H-pyrrolo[3,2-d]pyrimidinyl, 7H-purinyl, indolizinyl, pyrrolo[1,2-a]pyrimidinyl, pyrrolo[1,2-a]pyrazinyl, pyrrolo[1,2-c]pyriminyl, pyrrolo[1,2-b]pyridazinyl, imidazo[4,5-b]pyridinyl, pyrazolo[1,5-a]pyridinyl, imidazo[1,5-a]pyridinyl, imidazo[1,5-b]pyridazinyl, imidazo[1,2-a]pyridinyl, imidazo[1,2-b]pyridazinyl, imidazo[1,2-c]pyrimidinyl, imidazo[1,2-a]pyrimidinyl, [1,2,4]triazolo[4,3-a]pyridinyl, [1,2,3-triazolo[1,5-a]pyridinyl, quinolinyl, isoquinolinyl, cinnolinyl, quinazolinyl, quinoxalinyl, phthalazinyl, pyrido[2,3-b]pyrazinyl, pteridinyl, pyrido[3,4-d]pyridazinyl, 1,6-naphthyridinyl, 1,8-naphthyridinyl, 9H-carbazolyl, benzoxazolyl, dibenzofuranyl, benzothiphenyl, or dibenzothiophenyl.

8. The compound of any one of claims 1-7, having the structure of Formula (II): 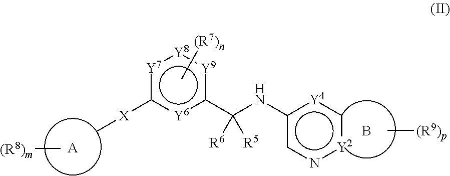
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
ring A is carbocycle or heterocycle;
Y² is C or N;
Y⁴ is a bond, —CR⁴═, or —N═;
R⁴ is H, halogen, cyano, alkyl, haloalkyl, alkoxy, —NH₂, —NHR⁹, or —NR⁹R⁹;
ring B is a 5- to 6-membered carbocycle or 5- to 6-membered heterocycle;
Y⁶, Y⁷, Y⁸, and Y⁹ are each, independently, —CH═, —CR⁷═, or N;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
R¹⁰ is H, alkyl, or haloalkyl;
R¹¹ is H, alkyl, or haloalkyl;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when ring B is a 6-membered carbocycle;
n is 0-5;
p is 0-5; and
q is 0-2.

9. The compound of any one of claims 1-8, having the structure of Formula (III): 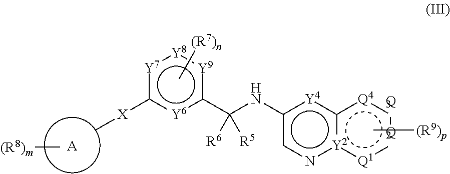
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
ring A is carbocycle or heterocycle;
Y² is C or N;
Y⁴ is a bond, —CR⁴═, or —N═;
R⁴ is H, halogen, cyano, alkyl, haloalkyl, alkoxy, —NH₂, —NHR⁹, or —NR⁹R⁹;
Q¹ and Q² are each, independently, C or N;
Q³ and Q⁴ are each, independently, a bond, C, N, S, or O;
Y⁶, Y⁷, Y⁸, and Y⁹ are each, independently, —CH═, —CR⁷═, or N;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
R¹⁰ is H, alkyl, or haloalkyl;
R¹¹ is H, alkyl, or haloalkyl;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when Q¹, Q², Q³, and Q⁴ are each C;
n is 0-5;
p is 0-5; and
q is 0-2.

10. The compound of any one of claims 1-9, having the structure of Formula (IV): 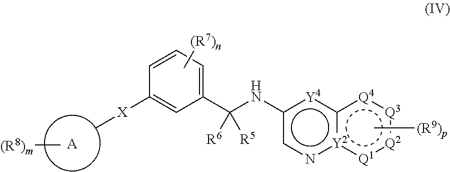
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
ring A is carbocycle or heterocycle;
Y² is C or N;
Y⁴ is a bond, —CR⁴═, or —N═;
R⁴ is H, halogen, cyano, alkyl, haloalkyl, alkoxy, —NH₂, —NHR⁹, or —NR⁹R⁹;
Q¹ and Q² are each, independently, C or N;
Q³ and Q⁴ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
R¹⁰ is H, alkyl, or haloalkyl;
R¹¹ is H, alkyl, or haloalkyl;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when Q¹, Q², Q³, and Q⁴ are each C;
n is 0-5;
p is 0-5; and
q is 0-2.

11. The compound of any one of claims 1-10, having the structure of Formula (V): 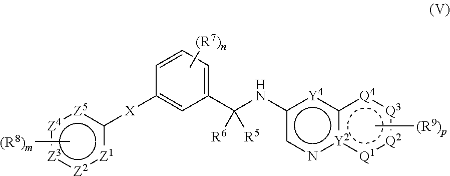
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y² is C or N;
Y⁴ is a bond, —CR⁴═, or —N═;
R⁴ is H, halogen, cyano, alkyl, haloalkyl, alkoxy, —NH₂, —NHR⁹, or —NR⁹R⁹;
Q¹ and Q² are each, independently, C or N;
Q³ and Q⁴ are each, independently, C, N, S, or O;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
R¹⁰ is H, alkyl, or haloalkyl;
R¹¹ is H, alkyl, or haloalkyl;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when Q¹, Q², Q³, and Q⁴ are each C;
n is 0-5;
p is 0-5; and
q is 0-2.

12. The compound of any one of claims 1-11, having the structure of Formula (VI): 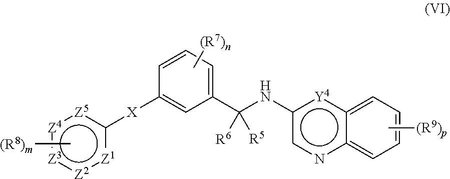
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y⁴ is a bond, —CR⁴═, or —N═;
R⁴ is H, halogen, cyano, alkyl, haloalkyl, alkoxy, —NH₂, —NHR⁹, or —NR⁹R⁹;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
R¹⁰ is H, alkyl, or haloalkyl;
R¹¹ is H, alkyl, or haloalkyl;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when Z³ is N;
n is 0-5;
p is 0-5; and
q is 0-2.

13. The compound of any one of claims 1-9, having the structure of Formula (VII): 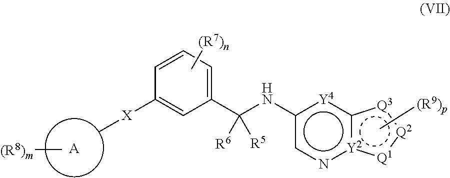
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
ring A is carbocycle or heterocycle;
Y² is C or N;
Y⁴ is a bond, —CR⁴═, or —N═;
R⁴ is H, halogen, cyano, alkyl, haloalkyl, alkoxy, —NH₂, —NHR⁹, or —NR⁹R⁹;
Q¹ and Q² are each, independently, C or N;
Q³ is C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
R¹⁰ is H, alkyl, or haloalkyl;
R¹¹ is H, alkyl, or haloalkyl;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5;
n is 0-5;
p is 0-5; and
q is 0-2.

14. The compound of any one of claims 1-9 or 13, having the structure of Formula (VIII): 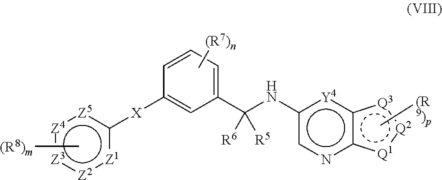
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y⁴ is a bond, —CR⁴═, or —N═;
R⁴ is H, halogen, cyano, alkyl, haloalkyl, alkoxy, —NH₂, —NHR⁹, or —NR⁹R⁹;
Q¹ and Q² are each, independently, C or N;
Q³ is C, N, S, or O;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
R¹⁰ is H, alkyl, or haloalkyl;
R¹¹ is H, alkyl, or haloalkyl;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5;
n is 0-5;
p is 0-5; and
q is 0-2.

15. The compound of any one of claims 1-9 or 13-14, having the structure of Formula (VIII-A): 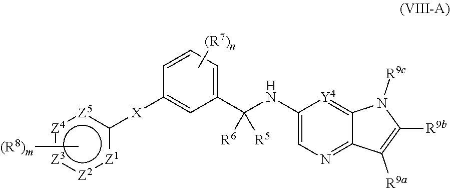
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y⁴ is —CR⁴═, or —N═;
R⁴ is H, halogen, cyano, alkyl, haloalkyl, alkoxy, —NH₂, —NHR⁹, or —NR⁹R⁹;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R^{9a} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;
R^{9b} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;
R^{9c} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;
R¹⁰ is H, alkyl, or haloalkyl;
R¹¹ is H, alkyl, or haloalkyl;
wherein R⁷, R⁸, R^{9a}, R^{9b}, and R^{9c} are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5;
n is 0-5; and
q is 0-2.

16. The compound of any one of claims 1-9 or 13-14, having the structure of Formula (VIII-B): 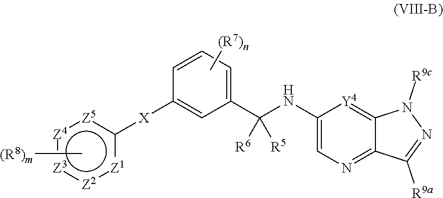
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y⁴ is —CR⁴═, or —N═;
R⁴ is H, halogen, cyano, alkyl, haloalkyl, alkoxy, —NH₂, —NHR⁹, or —NR⁹R⁹;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R^{9a} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;
R^{9c} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;
R¹⁰ is H, alkyl, or haloalkyl;
R¹¹ is H, alkyl, or haloalkyl;
wherein R⁷, R⁸, R^{9a}, and R^{9c} are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5;
n is 0-5; and
q is 0-2.

17. The compound of any one of claims 1-9 or 13-14, having the structure of Formula (VIII-C): 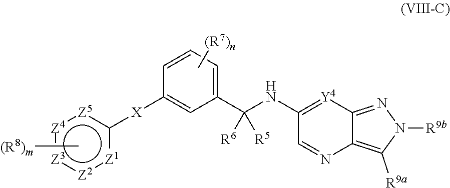
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y⁴ is —CR⁴═, or —N═;
R⁴ is H, halogen, cyano, alkyl, haloalkyl, alkoxy, —NH₂, —NHR⁹, or —NR⁹R⁹;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R^{9a} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;
R^{9b} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;
R¹⁰ is H, alkyl, or haloalkyl;
R¹¹ is H, alkyl, or haloalkyl;
wherein R⁷, R⁸, R^{9a}, and R^{9b} are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5;
n is 0-5; and
q is 0-2.

18. The compound of any one of claims 1-9 or 13-14, having the structure of Formula (VIII-D): 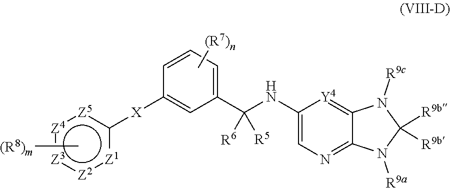
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y⁴ is —CR⁴═, or —N═;
R⁴ is H, halogen, cyano, alkyl, haloalkyl, alkoxy, —NH₂, —NHR⁹, or —NR⁹R⁹;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R^{9a} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;
R^{9b′}, and R^{9b″} are each, independently, halogen, cyano, alkyl, alkenyl, alkynyl, carbocycle, heterocycle, carbocyclealkyl, heterocyclealkyl, or R^{9b′} and R^{9b″} together form ═O, or R^{9b′} and R^{9b″} together with the carbon to which they are attached form a 3-7 membered carbocycle or heterocycle;
R^{9c} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;
R¹⁰ is H, alkyl, or haloalkyl;
R¹¹ is H, alkyl, or haloalkyl;
wherein R⁷, R⁸, R^{9a}, R^{9b′}, R^{9b″}, and R^{9c} are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5;
n is 0-5; and
q is 0-2.

19. The compound of any one of claims 1-9 or 13-14, having the structure of Formula (VIII-E) 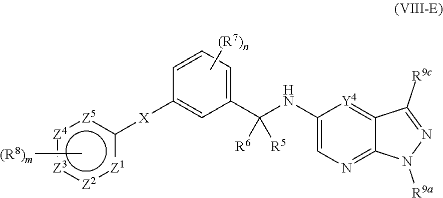
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y⁴ is —CR⁴═, or —N═;
R⁴ is H, halogen, cyano, alkyl, haloalkyl, alkoxy, —NH₂, —NHR⁹, or —NR⁹R⁹;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R^{9a} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;
R^{9c} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;
R¹⁰ is H, alkyl, or haloalkyl;
R¹¹ is H, alkyl, or haloalkyl;
wherein R⁷, R⁸, R^{9a}, and R^{9c} are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5;
n is 0-5; and
q is 0-2.

20. The compound of any one of claims 1-9 or 13-14, having the structure of Formula (VIII-F): 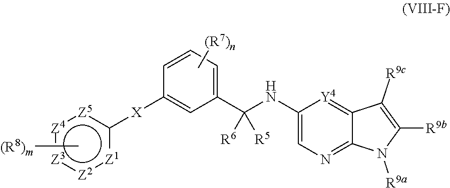
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y⁴ is —CR⁴═, or —N═;
R⁴ is H, halogen, cyano, alkyl, haloalkyl, alkoxy, —NH₂, —NHR⁹, or —NR⁹R⁹;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R^{9a} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;
R^{9b} and R^{9c} are each, independently, H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;
R¹⁰ is H, alkyl, or haloalkyl;
R¹¹ is H, alkyl, or haloalkyl;
wherein R⁷, R⁸, R^{9a}, R^{9b}, and R^{9c} are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5;
n is 0-5; and
q is 0-2.

21. The compound of any one of claims 1-9 or 13-14, having the structure of Formula (VIII-G): 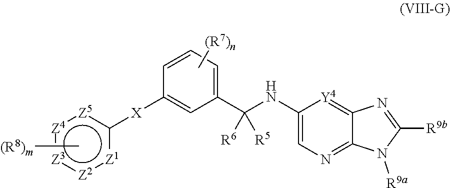
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y⁴ is —CR⁴═, or —N═;
R⁴ is H, halogen, cyano, alkyl, haloalkyl, alkoxy, —NH₂, —NHR⁹, or —NR⁹R⁹;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R^{9a} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;
R^{9b} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;
R¹⁰ is H, alkyl, or haloalkyl;
R¹¹ is H, alkyl, or haloalkyl;
wherein R⁷, R⁸, R^{9a}, and R^{9b} are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5;
n is 0-5; and
q is 0-2.

22. The compound of any one of claims 1-9 or 13-14, having the structure of Formula (VIII-H): 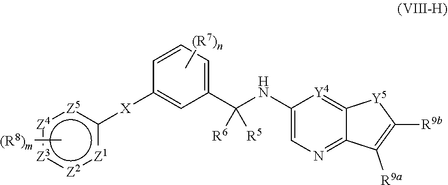
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y⁴ is —CR⁴═, or —N═;
Y⁵ is O, or S;
R⁴ is H, halogen, cyano, alkyl, haloalkyl, alkoxy, —NH₂, —NHR⁹, or —NR⁹R⁹;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R^{9a} and R^{9b} are each, independently, H, halogen, cyano, —OR^{a}, —S(O)_{q}R^{a}, —S(O)_{q}NR^{a}R^{b}, —NR^{a}S(O)_{c}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;
R¹⁰ is H, alkyl, or haloalkyl;
R¹¹ is H, alkyl, or haloalkyl;
wherein R⁷, R⁸, R^{9a}, and R^{9b} are each, independently, optionally substituted with one or more R;
R is —OR^{a}, —C(O)^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5;
n is 0-5; and
q is 0-2.

23. The compound of any one of claims 1-7, having the structure of Formula (IX): 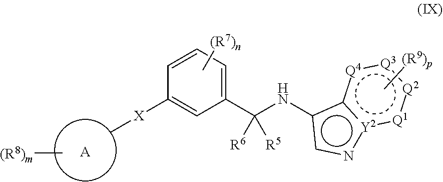
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
ring A is carbocycle or heterocycle;
Y² is C or N;
Q¹ and Q² are each, independently, C or N;
Q³ and Q⁴ are each, independently, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when Q¹, Q², Q³, and Q⁴ are each C;
n is 0-5;
p is 0-5; and
q is 0-2.

24. The compound of of any one of claims 1-7 or 23, having the structure of Formula (X): 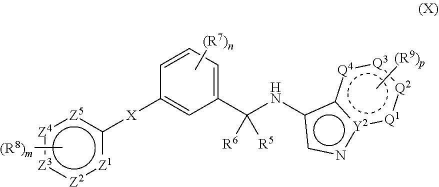
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y² is C or N;
Q¹ and Q² are each, independently, C or N;
Q³ and Q⁴ are each, independently, C, N, S, or O;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when Q¹, Q², Q³, and Q⁴ are each C;
n is 0-5;
p is 0-5; and
q is 0-2.

25. The compound of of any one of claims 1-7 or 23-24, having the structure of Formula (XI): 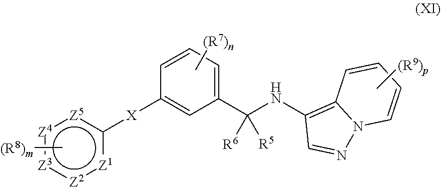
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 1-5;
n is 0-5;
p is 0-5; and
q is 0-2.

26. The compound of any one of claims 1-11 or 23-24, or a pharmaceutically acceptable isomer, racemate, hydrate, solvate, tautomer, isotope, or salt thereof, wherein Y² is C.

27. The compound of any one of claims 1-11 or 23-24, or a pharmaceutically acceptable isomer, racemate, hydrate, solvate, tautomer, isotope, or salt thereof, wherein Y² is N.

28. The compound of any one of claims 1-22, or a pharmaceutically acceptable isomer, racemate, hydrate, solvate, tautomer, isotope, or salt thereof, wherein Y⁴ is C.

29. The compound of any one of claims 1-22, or a pharmaceutically acceptable isomer, racemate, hydrate, solvate, tautomer, isotope, or salt thereof, wherein Y⁴ is N.

30. The compound of any one of claims 1-7, having the structure of Formula (XII): 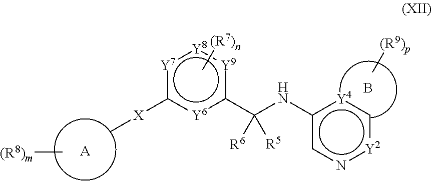
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
ring A is carbocycle or heterocycle;
Y² is a bond, —CR²═, or —N═;
Y⁴ is C or N;
R² is H, halogen, cyano, alkyl, haloalkyl, alkoxy, —NH₂, —NHR⁹, or —NR⁹R⁹;
ring B is a 5- to 6-membered carbocycle or 5- to 6-membered heterocycle, wherein ring B is substituted by (R⁹)ₚ;
Y⁶, Y⁷, Y⁸, and Y⁹ are each, independently, —CH═, —CR⁷═, or N;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when ring B is a 6-membered carbocycle;
n is 0-5;
p is 0-5; and
q is 0-2.

31. The compound of any one of claims 1-7 or 30, having the structure of Formula (XIII): 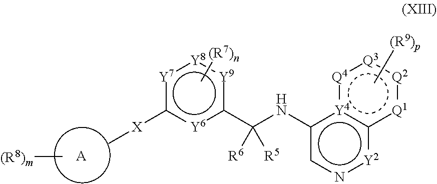
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
ring A is carbocycle or heterocycle;
Y² is a bond, —CR²═, or —N═;
Y⁴ is C or N;
R² is H, halogen, cyano, alkyl, haloalkyl, alkoxy, —NH₂, —NHR⁹, or —NR⁹R⁹;
Q¹ and Q² are each, independently, C or N;
Q³ and Q⁴ are each, independently, a bond, C, N, S, or O;
Y⁶, Y⁷, Y⁸, and Y⁹ are each, independently, —CH═, —CR⁷═, or N;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when Q¹, Q², Q³, and Q⁴ are each C;
n is 0-5;
p is 0-5; and
q is 0-2.

32. The compound of any one of claims 1-7 or 30-31, having the structure of Formula (XIV): 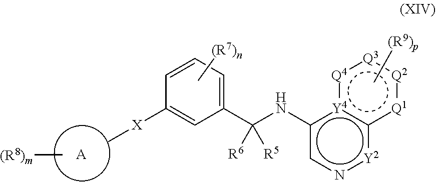
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
ring A is carbocycle or heterocycle;
Y² is a bond, —CR²═, or —N═;
Y⁴ is C or N;
R² is H, halogen, cyano, alkyl, haloalkyl, alkoxy, —NH₂, —NHR⁹, or —NR⁹R⁹;
Q¹ and Q² are each, independently, C or N;
Q³ and Q⁴ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when Q¹, Q², Q³, and Q⁴ are each C;
n is 0-5;
p is 0-5; and
q is 0-2.

33. The compound of any one of claims 1-7 or 30-32, having the structure of Formula (XV): 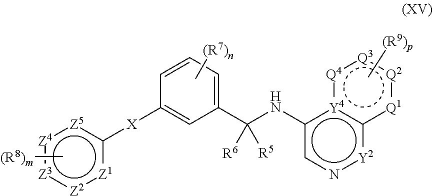
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y² is a bond, —CR²═, or —N═;
Y⁴ is C or N;
R² is H, halogen, cyano, alkyl, haloalkyl, alkoxy, —NH₂, —NHR⁹, or —NR⁹R⁹;
Q¹ and Q² are each, independently, C or N;
Q³ and Q⁴ are each, independently, C, N, S, or O;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when Q¹, Q², Q³, and Q⁴ are each C;
n is 0-5;
p is 0-5; and
q is 0-2.

34. The compound of any one of claims 1-7 or 30-33, having the structure of Formula (XVI): 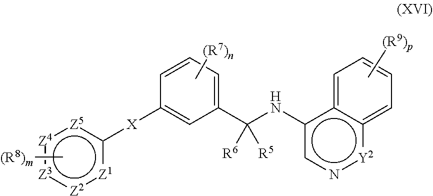
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y² is a bond, —CR²═, or —N═;
R² is H, halogen, cyano, alkyl, haloalkyl, alkoxy, —NH₂, —NHR⁹, or —NR⁹R⁹;
Q¹ and Q² are each, independently, C or N;
Q³ and Q⁴ are each, independently, C, N, S, or O;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when Q¹, Q², Q³, and Q⁴ are each C;
n is 0-5;
p is 0-5; and
q is 0-2.

35. The compound of any one of claims 1-7, having the structure of Formula (XVII): 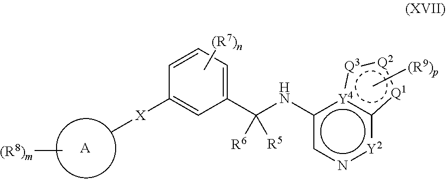
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
ring A is carbocycle or heterocycle;
Y² is a bond, —CR²═, or —N═;
Y⁴ is C or N;
R² is H, halogen, cyano, alkyl, haloalkyl, alkoxy, —NH₂, —NHR⁹, or —NR⁹R⁹;
Q¹ and Q² are each, independently, C or N;
Q³ is C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5;
n is 0-5;
p is 0-5; and
q is 0-2.

36. The compound of any one of claims 1-7 or 35, having the structure of Formula (XVIII): 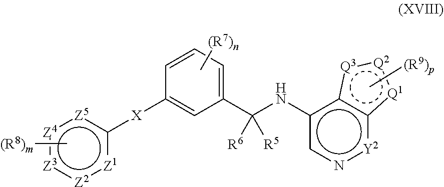
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y² is a bond, —CR²═, or —N═;
R² is H, halogen, cyano, alkyl, haloalkyl, alkoxy, —NH₂, —NHR⁹, or —NR⁹R⁹;
Q¹ and Q² are each, independently, C or N;
Q³ is C, N, S, or O;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5;
n is 0-5;
p is 0-5; and
q is 0-2.

37. The compound of any one of claims 1-7 or 35-36; having the structure of Formula (XVIII-A): 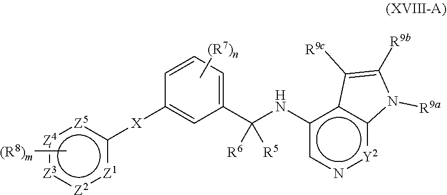
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y² is a bond, —CR²═, or —N═;
R² is H, halogen, cyano, alkyl, haloalkyl, alkoxy, —NH₂, —NHR⁹, or —NR⁹R⁹;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R^{9a} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;
R^{9b} and R^{9c} are each, independently, H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;
wherein R⁷, R⁸, R^{9a}, R^{9b}, and R^{9c} are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5;
n is 0-5; and
q is 0-2.

38. The compound of any one of claims 1-7 or 35-36, having the structure of Formula (XVIII-B): 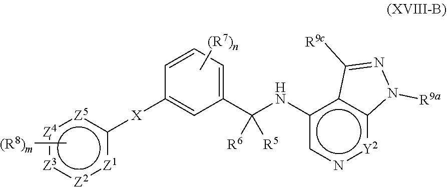
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y² is a bond, —CR²═, or —N═;
R² is H, halogen, cyano, alkyl, haloalkyl, alkoxy, —NH₂, —NHR⁹, or —NR⁹R⁹;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl;
R^{9a} is H, —S(O)_{q}R^{a}, —S(O)_{q}NR^{a}R^{b}, alkyl, alkenyl, alkynyl, haloalkyl, carbocycle, carbocyclealkyl, heterocycle, or heterocyclealkyl;
R^{9c} is H, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, or heterocyclealkyl;
wherein R⁷, R⁸, and R⁹, R^{9a}, and R^{9c} are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5;
n is 0-5; and
q is 0-2.

39. The compound of any one of claim 1-7, having the structure of Formula (XIX): 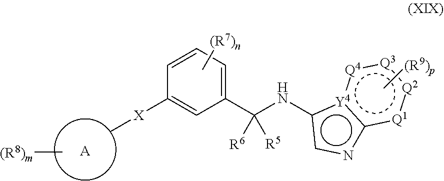
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
ring A is carbocycle or heterocycle;
Y⁴ is C or N;
Q¹ and Q² are each, independently, C or N;
Q³ and Q⁴ are each, independently, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when Q¹, Q², Q³, and Q⁴ are each C;
n is 0-5;
p is 0-5; and
q is 0-2.

40. The compound of any one of claims 1-7 or 39, having the structure of Formula (XX): 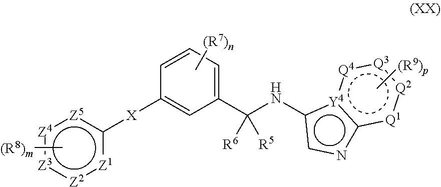
or a pharmaceutically acceptable isomer, racemate, hydrate, solvate, tautomer, isotope, or salt thereof, wherein:
X is —C(O)NH—, —C(R¹⁰R¹¹)C(O)NH—, —NHC(O)NH—, or —NHC(O)—;
Y⁴ is C or N;
Q¹ and Q² are each, independently, C or N;
Q³ and Q⁴ are each, independently, C, N, S, or O;
Z¹, Z², Z³, Z⁴, and Z⁵ are each, independently, a bond, C, N, S, or O;
R⁵ is H, alkyl, haloalkyl, or hydroxyalkyl;
R⁶ is H, alkyl, haloalkyl, or hydroxyalkyl;
or R⁵ and R⁶, together with the carbon atom they are attached to, form a 3- to 5-membered carbocycle or heterocycle;
R⁷ is, at each occurrence, independently halogen, cyano, alkyl, haloalkyl, alkoxy, or haloalkoxy;
or R⁶ and one R⁷, together with the atoms to which they are attached, form a 5- to 6-membered carbocycle;
R⁸ is, at each occurrence, independently halogen, cyano, —OR^{a}, —S(O)_{q}R^{a}, —S(O)_{q}NR^{a}R^{b}, —NR^{a}S(O)_{q}R^{b}, —C(O)R^{a}, —OC(O)R^{a}, —C(O)OR^{a}, —OC(O)OR^{a}, —C(O)NR^{a}R^{b}, —NR^{a}R^{b}, —OC(O)NR^{a}R^{b}, —NR^{a}C(O)R^{b}, —NR^{a}C(O)OR^{b}, C₁₋₄ alkyl, C₁₋₄ alkenyl, C₁₋₄ alkynyl, carbocycle, carbocyclealkyl, heterocycle, heterocyclealkyl, or two R⁸ together form ═O;
R⁹ is, at each occurrence, halogen, cyano, —OR^{a}, —S(O)_{q}R^{a}, —S(O)_{q}NR^{a}R^{b}, —NR^{a}S(O)_{q}R^{b}, —NR^{a}R^{b}, —OC(O)NR^{a}R^{b}, —NR^{a}C(O)R^{b}, —NR^{a}C(O)OR^{b}, alkyl, alkenyl, alkynyl, haloalkyl, carbocycle, carbocyclealkyl, heterocycle, heterocyclealkyl, or two R⁹ together form ═O;
wherein R⁷, R⁸, and R⁹ are each, independently, optionally substituted with one or more R;
R is —OR^{a}, —C(O)R^{a}, —NR^{a}R^{b}, halogen, cyano, alkyl, haloalkyl, alkoxy, carbocycle, heterocycle, carbocyclalkyl, or heterocyclealkyl;
R^{a} is H, alkyl, haloalkyl, carbocycle, heterocycle, carbocyclealkyl, or heterocyclealkyl;
R^{b} is H, alkyl, haloalkyl, carbocycle, heterocycle, carbocyclealkyl, or heterocyclealkyl;
or R^{a} and R^{b}, together with the nitrogen atom to which they are attached, form C₄₋₈ cycloalkyl, or 4- to 8-membered saturated heterocycle;
m is 0-5, wherein m is 1-5 when Q¹, Q², Q³, and Q⁴ are each C;
n is 0-5;
p is 0-5; and
q is 0-2.

41. The compound of any one of claims 1-40, or a pharmaceutically acceptable isomer, racemate, hydrate, solvate, tautomer, isotope, or salt thereof, wherein X is —C(O)NH—.

42. The compound of any one of claims 1-40, or a pharmaceutically acceptable isomer, racemate, hydrate, solvate, tautomer, isotope, or salt thereof, wherein X is —C(R¹⁰R¹¹)C(O)NH—.

43. The compound of any one of claims 1-40, or a pharmaceutically acceptable isomer, racemate, hydrate, solvate, tautomer, isotope, or salt thereof, wherein X is —NHC(O)NH—.

44. The compound of any one of claims 1-40, or a pharmaceutically acceptable isomer, racemate, hydrate, solvate, tautomer, isotope, or salt thereof, wherein X is —NHC(O)—.

45. The compound of any one of claims 1-44, or a pharmaceutically acceptable isomer, racemate, hydrate, solvate, tautomer, isotope, or salt thereof, wherein R⁵ is H and R⁶ is alkyl.

46. The compound of claim 45, or a pharmaceutically acceptable isomer, racemate, hydrate, solvate, tautomer, isotope, or salt thereof, wherein R⁶ is methyl.

47. The compound of any one of claims 1-46, or a pharmaceutically acceptable isomer, racemate, hydrate, solvate, tautomer, isotope, or salt thereof, wherein n is 0.

48. The compound of any one of claims 1-46, or a pharmaceutically acceptable isomer, racemate, hydrate, solvate, tautomer, isotope, or salt thereof, wherein n is 1 or 2.

49. The compound of any one of claims 1-48, or a pharmaceutically acceptable isomer, racemate, hydrate, solvate, tautomer, isotope, or salt thereof, wherein m is 0.

50. The compound of any one of claims 1-48, or a pharmaceutically acceptable isomer, racemate, hydrate, solvate, tautomer, isotope, or salt thereof, wherein m is 1 or 2.

51. The compound of claim 50, or a pharmaceutically acceptable isomer, racemate, hydrate, solvate, tautomer, isotope, or salt thereof, wherein at least one R⁸ is alkyl.

52. The compound of claim 51, or a pharmaceutically acceptable isomer, racemate, hydrate, solvate, tautomer, isotope, or salt thereof, wherein at least one R⁸ is methyl, ethyl, iso-propyl, n-propyl, or t-butyl.

53. The compound of claim 50, or a pharmaceutically acceptable isomer, racemate, hydrate, solvate, tautomer, isotope, or salt thereof, wherein at least one R⁸ is alkyl substituted with halogen.

54. The compound of claim 53, or a pharmaceutically acceptable isomer, racemate, hydrate, solvate, tautomer, isotope, or salt thereof, wherein at least one R⁸ is difluoromethyl, trifluoromethyl, 2-fluoroethyl, or 2,2-difluoroethyl.

55. The compound of claim 50, or a pharmaceutically acceptable isomer, racemate, hydrate, solvate, tautomer, isotope, or salt thereof, wherein at least one R⁸ is alkyl substituted with —OR^{a} and R^{a} is H or alkyl.

56. The compound of claim 55, or a pharmaceutically acceptable isomer, racemate, hydrate, solvate, tautomer, isotope, or salt thereof, wherein at least one R⁸ is hydroxymethyl, 2-hydroxyethyl, hydroxybutyl, or methoxymethyl.

57. The compound of claim 50, or a pharmaceutically acceptable isomer, racemate, hydrate, solvate, tautomer, isotope, or salt thereof, wherein at least one R⁸ is carbocycle.

58. The compound of claim 57, or a pharmaceutically acceptable isomer, racemate, hydrate, solvate, tautomer, isotope, or salt thereof, wherein at least one R⁸ is cyclopropyl or cyclobutyl.

59. The compound of claim 50, or a pharmaceutically acceptable isomer, racemate, hydrate, solvate, tautomer, isotope, or salt thereof, wherein at least one of R⁸ is heterocycle.

60. The compound of claim 50, or a pharmaceutically acceptable isomer, racemate, hydrate, solvate, tautomer, isotope, or salt thereof, wherein at least one R⁸ is —OR^{a}.

61. The compound of claim 60, or a pharmaceutically acceptable isomer, racemate, hydrate, solvate, tautomer, isotope, or salt thereof, wherein at least one R^{a} is alkyl.

62. The compound of claim 60, or a pharmaceutically acceptable isomer, racemate, hydrate, solvate, tautomer, isotope, or salt thereof, wherein at least one R^{a} is haloalkyl.

63. The compound of claim 60, or a pharmaceutically acceptable isomer, racemate, hydrate, solvate, tautomer, isotope, or salt thereof, wherein at least one R^{a} is carbocycle.

64. The compound of claim 50, or a pharmaceutically acceptable isomer, racemate, hydrate, solvate, tautomer, isotope, or salt thereof, wherein at least one R⁸ is —NR^{a}R^{b}.

65. The compound of claim 64, or a pharmaceutically acceptable isomer, racemate, hydrate, solvate, tautomer, isotope, or salt thereof, wherein at least one R^{a} is H and at least one R^{b} is alkyl.

66. The compound of claim 64, or a pharmaceutically acceptable isomer, racemate, hydrate, solvate, tautomer, isotope, or salt thereof, wherein at least one R^{a} is H and at least one R^{b} is haloalkyl.

67. The compound of claim 50, or a pharmaceutically acceptable isomer, racemate, hydrate, solvate, tautomer, isotope, or salt thereof, wherein at least one R⁸ is cyano.

68. The compound of claim 50, or a pharmaceutically acceptable isomer, racemate, hydrate, solvate, tautomer, isotope, or salt thereof, wherein at least one R⁸ is halogen.

69. The compound of claim 68, or a pharmaceutically acceptable isomer, racemate, hydrate, solvate, tautomer, isotope, or salt thereof, wherein at least one R⁸ is Cl.

70. The compound of any one of claims 1-69, or a pharmaceutically acceptable isomer, racemate, hydrate, solvate, tautomer, isotope, or salt thereof, wherein p is 0.

71. The compound of any one of claims 1-69, or a pharmaceutically acceptable isomer, racemate, hydrate, solvate, tautomer, isotope, or salt thereof, wherein p is 1 or 2.

72. The compound of claim 71, or a pharmaceutically acceptable isomer, racemate, hydrate, solvate, tautomer, isotope, or salt thereof, wherein at least one R⁹ is halogen.

73. The compound of claim 72, or a pharmaceutically acceptable isomer, racemate, hydrate, solvate, tautomer, isotope, or salt thereof, wherein at least one R⁹ is Cl or Br.

74. The compound of claim 71, or a pharmaceutically acceptable isomer, racemate, hydrate, solvate, tautomer, isotope, or salt thereof, wherein at least one R⁹ is alkyl.

75. The compound of claim 74, or a pharmaceutically acceptable isomer, racemate, hydrate, solvate, tautomer, isotope, or salt thereof, wherein at least one R⁹ is methyl or ethyl.

76. The compound of claim 74 or 75, or a pharmaceutically acceptable isomer, racemate, hydrate, solvate, tautomer, isotope, or salt thereof, wherein at least one R⁹ is optionally substituted with carbocycle or heterocycle.

77. The compound of claim 74 or 75, or a pharmaceutically acceptable isomer, racemate, hydrate, solvate, tautomer, isotope, or salt thereof, wherein at least one R⁹ is substituted with —OR^{a}.

78. The compound of claim 71, or a pharmaceutically acceptable isomer, racemate, hydrate, solvate, tautomer, isotope, or salt thereof, wherein at least one R⁹ is carbocycle.

79. The compound of claim 78, or a pharmaceutically acceptable isomer, racemate, hydrate, solvate, tautomer, isotope, or salt thereof, wherein at least one R⁹ is phenyl.

80. The compound of claim 78 or 79, or a pharmaceutically acceptable isomer, racemate, hydrate, solvate, tautomer, isotope, or salt thereof, wherein at least one R⁹ is substituted with —OR^{a}.

81. The compound of claim 80, or a pharmaceutically acceptable isomer, racemate, hydrate, solvate, tautomer, isotope, or salt thereof, wherein R^{a} is, at each occurrence, independently H or alkyl.

82. The compound of claim 71, or a pharmaceutically acceptable isomer, racemate, hydrate, solvate, tautomer, isotope, or salt thereof, wherein at least one R⁹ is heterocycle.

83. The compound of claim 71, or a pharmaceutically acceptable isomer, racemate, hydrate, solvate, tautomer, isotope, or salt thereof, wherein at least one R⁹ is —OR^{a}.

84. The compound of claim 83, or a pharmaceutically acceptable isomer, racemate, hydrate, solvate, tautomer, isotope, or salt thereof, wherein R^{a} is, at each occurrence, independently H or alkyl.

85. The compound of claim 71, or a pharmaceutically acceptable isomer, racemate, hydrate, solvate, tautomer, isotope, or salt thereof, wherein two R⁹ together form ═O.

86. A compound having a structure listed in Table 5, or a pharmaceutically acceptable isomer, racemate, hydrate, solvate, tautomer, isotope, or salt thereof.

87. A substantially enantiomerically pure form of a compound having a structure listed in Table 5 or a pharmaceutically acceptable isomer, racemate, hydrate, solvate, tautomer, isotope, or salt thereof.

88. A composition comprising a compound of any one of claims 1-87, or a pharmaceutically acceptable isomer, racemate, hydrate, solvate, tautomer, isotope, or salt thereof, and a pharmaceutically acceptable carrier, diluent, or excipient.

89. A method for inhibiting PDGF receptor α, comprising contacting the PDGF receptor α with an effective amount of a compound of any one of claims 1-87, or a pharmaceutically acceptable isomer, racemate, hydrate, solvate, tautomer, isotope, or salt thereof, or a composition comprising the same.

90. A method for inhibiting PDGF receptor β, comprising contacting the PDGF receptor β with an effective amount of a compound of any one of claims 1-87, or a pharmaceutically acceptable isomer, racemate, hydrate, solvate, tautomer, isotope, or salt thereof, or a composition comprising the same.

91. A method for treating a PDGF receptor α-dependent condition, comprising administering to a subject in need thereof an effective amount of a compound of any one of claims 1-87, or a pharmaceutically acceptable isomer, racemate, hydrate, solvate, tautomer, isotope, or salt thereof, or a composition comprising the same.

92. A method for treating a PDGF receptor β-dependent condition, comprising administering to a subject in need thereof an effective amount of a compound of any one of claims 1-87, or a pharmaceutically acceptable isomer, racemate, hydrate, solvate, tautomer, isotope, or salt thereof, or a composition comprising the same.

93. A method for treating a pulmonary disorder, comprising administering to a subject in need thereof an effective amount of a compound of any one of claims 1-87, or a pharmaceutically acceptable isomer, racemate, hydrate, solvate, tautomer, isotope, or salt thereof, or a composition comprising the same.

94. The method of claim 93, wherein the pulmonary disorder is pulmonary hypertension.

95. The method of claim 94, wherein the pulmonary hypertension is pulmonary arterial hypertension.

96. The method of claim 95, wherein the pulmonary arterial hypertension is primary PAH, idiopathic PAH, heritable PAH, refractory PAH, BMPR2, AL 1, endoglin associated with hereditary hemorrhagic telangiectasia, endoglin not associated with hereditary hemorrhagic telangiectasia, drug-induced PAH, or toxin-induced PAH.

97. The method of claim 95, wherein the pulmonary arterial hypertension is associated with systemic sclerosis, mixed connective tissue disease, HIV, hepatitis, or portal hypertension.

98. The method of claim 94, wherein the pulmonary hypertension is associated with myeloproliferative disorders.

99. The method of claim 98, wherein the myeloproliferative disorder associated pulmonary hypertension is Group 5 PAH.

100. A method for treating a disease associated with tissue fibrosis, comprising administering to a subject in need thereof an effective amount of a compound of any one of claims 1-87, or a pharmaceutically acceptable isomer, racemate, hydrate, solvate, tautomer, isotope, or salt thereof, or a composition comprising the same.

101. The method of claim 100, wherein the disease associated with tissue fibrosis is systemic sclerosis, interstitial lung disease, bronchiolitis obliterans with organizing pneumonia (BOOP), acute lung injury, glomerulonephritis, focal segmental glomerulosclerosis, stroke, or radiation induced fibrosis.

102. A method for treating solid tumors, comprising administering to a subject in need thereof an effective amount of a compound of any one of claims 1-87, or a pharmaceutically acceptable isomer, racemate, hydrate, solvate, tautomer, isotope, or salt thereof, or a composition comprising the same.
